SEQUENCE LISTING (3 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 1255 aa, Homo sapiens [sequence omitted]

SEQ ID NO: 3 - protein, 1210 aa, Homo sapiens [sequence omitted]

(19) United States
(12) Patent Application Publication
(10) Pub. No.: US 2019/0119284 A1
(43) Pub. Date: Apr. 25, 2019

(54) ErbB Inhibitors and Uses Thereof

(71) Applicants: The Regents of the University of California, Oakland, CA (US); The Scripps Research Institute, La Jolla, CA (US)

(72) Inventors: Christopher Novotny, San Francisco, CA (US); Kevan M. Shokat, San Francisco, CA (US); Weijun Shen, San Diego, CA (US)

(21) Appl. No.: 16/094,729

(22) PCT Filed: Apr. 19, 2017
(86) PCT No.: PCT/US2017/028437

§ 371 Date: Oct. 18, 2018

Related U.S. Application Data
(60) Provisional application No. 62/324,864, filed on Apr. 19, 2016.

(51) Int. Cl. C07D 487/04; A61K 45/06; A61P 35/00; A61K 47/64; A61K 31/519

(52) U.S. Cl. CPC C07D 487/04; A61K 45/06; A61K 31/519; A61K 47/6425; A61P 35/00

(57) ABSTRACT

Described herein, inter alia, are compositions of ErbB modulators and methods of using the same.

### CROSS-REFERENCES TO RELATED APPLICATIONS

This application claims the benefit of U.S. Provisional Application No. 62/324,864, filed Apr. 19, 2016, which is incorporated herein by reference in entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 48536-586001WO_ST25.txt, created Apr. 19, 2017, 33,272 bytes, machine format IBM-PC, MS Windows operating system, is hereby incorporated by reference.

### BACKGROUND

Signaling from the epidermal growth factor receptor (EGFR or HER) family of receptor tyrosine kinases (RTK) is dependent on a well-orchestrated series of interactions between family members to form either homo- or heterodimers. This dimerization process allows the intracellular kinase domains to form an asymmetric dimer in which the C-terminal domain of the activator kinase binds to the N-terminal portion of the receiver kinase to stabilize it in an active conformation. The receiver kinase then phosphorylates tyrosine residues on the C-terminal tails of the kinases to recruit and activate downstream signaling components, most notably those involved in pro-growth and survival pathways. Because of this, the improper activation of the EGFR family of kinases, either by mutation or overexpression, is observed in a variety of cancers. Interestingly, cell culture studies suggest that rather than causing escape from the biological mechanism of regulation, oncogenic activation alters the equilibrium between active and in-active states to favor the improper dimerization and activation of these receptors. This dependence on dimerization is particularly evident in HER2 overexpressing breast cancers that are dependent on the presence of HER3. Within the EGFR family, HER2 and HER3 are unique. HER3 is classified as a pseudokinase with only residual kinase activity, whereas HER2 has no known activating ligand but is constitutively able to dimerize with other active family members. In this way HER2 and HER3 together form a functional RTK unit, with HER3 responding to activating ligands such as neuregulin (NRG), HER2 providing the intracellular kinase activity, and both intracellular domains providing phosphorylation sites. Additionally, HER2 and HER3 are each other's preferred heterodimerization partners and also form the most mitogenic complex among all possible EGFR family dimers. Because of this co-dependence, HER3 is important for the formation, proliferation, and survival of HER2 overexpressing tumors. Disclosed herein, inter alia, are solutions to these and other problems in the art.

### BRIEF SUMMARY OF THE INVENTION

In an aspect is provided a compound having the formula:

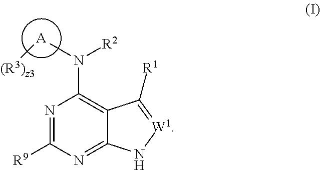

Ring A is aryl or heteroaryl. W¹ is N or C(H). R¹ is hydrogen, -L¹-L²-E, substituted or unsubstituted aryl, or substituted or unsubstituted heteroaryl. R² is hydrogen, —CX²₃, —CHX²₂, —CH₂X², substituted or unsubstituted alkyl, or substituted or unsubstituted heteroalkyl. R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —N R^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol z3 is an integer from 0 to 4. L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, (O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. E is an electrophilic moiety. Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl. R^{9A} and R^{9B} substituents bonded to the same nitrogen atom may optionally be joined to form a substituted or unsubstituted heterocycloalkyl or substituted or unsubstituted heteroaryl. Each X, X³, X⁶, X⁷, and X⁹ is independently —F, —Cl, —Br, or —I. The symbols n3, n6, n7, and n9 are independently an integer from 0 to 4. The symbols m3, m6, m7, m9, v3, v6, v7, and v9 are independently an integer from 1 to 2.

In another aspect is provided a pharmaceutical composition including a pharmaceutically acceptable excipient and a compound, or pharmaceutically acceptable salt thereof, as described herein, including embodiments.

In an aspect is provided a method of treating cancer in a patient in need of such treatment, the method including administering a therapeutically effective amount of a compound, or a pharmaceutically acceptable salt thereof, described herein.

In an aspect is provided a method of treating a disease associated with EGFR activity, HER2 activity, HER3 activity, HER4 activity, c-MET activity, PI3K activity, MEK activity, MAPK activity, RAF activity, BRAF activity, AKT activity, RAS activity, KRAS activity, heregulin activity, or neuregulin activity in a patient in need of such treatment, the method including administering a therapeutically effective amount of a compound described herein, or a pharmaceutically acceptable salt thereof.

In an aspect is provided a method of treating psoriasis, eczema, or atherosclerosis, in a patient in need of such treatment, the method including administering a therapeutically effective amount of a compound, or a pharmaceutically acceptable salt thereof, described herein.

In an aspect is provided a method of inhibiting an ERBB (e.g., ERBB1, ERBB2, ERBB3, or ERBB4) activity, the method including contacting ERBB (e.g., ERBB1, ERBB2, ERBB3, or ERBB4) with an effective amount of a compound described herein, or a pharmaceutically acceptable salt thereof.

In an aspect is provided a method of inhibiting HER2 activity, the method including contacting HER2 with an effective amount of a compound described herein, or a pharmaceutically acceptable salt thereof.

In an aspect is provided a method of inhibiting EGFR activity, the method including contacting EGFR with an effective amount of a compound described herein, or a pharmaceutically acceptable salt thereof.

In an aspect is provided an EGFR protein covalently bonded to a compound (e.g., a compound described herein, an EGFR inhibitor).

In an aspect is provided a HER2 protein covalently bonded to a compound (e.g., a compound described herein, a HER2 inhibitor).

In an aspect is provided an ERBB (e.g., ERBB1, ERBB2, ERBB3, or ERBB4) protein covalently bonded to a compound (e.g., a compound described herein, an ERBB (e.g., ERBB1, ERBB2, ERBB3, or ERBB4) inhibitor).

### BRIEF DESCRIPTION OF THE DRAWINGS

FIGS. 1A-1E. NRG rescues HER2 over-expressing cancer cells from HER2 inhibitors. FIG. 1A: 72 h Proliferation of SK-BR-3 and BT-474 cells treated with a dose-response of lapatinib in the presence or absence of NRG (mean±SD, n=3). FIG 1B: Same experiment as depicted in FIG. 1A, but with the inhibitor TAK-285. FIG. 1C: The ability of NRG to rescue SK-BR-3 and BT-474 cell proliferation from HER2 inhibitors is dose dependent. Cells were treated with 1 μM of the indicated inhibitor in the presence of varying concentrations of NRG, and proliferation was read out after 72 h (mean±SD, n=3). FIG. 1D: HER2 HER3 signaling was evaluated over a time-course in SK-BR-3 cells treated with either lapatinib, NRG, or both, The addition of NRG rescues p-HER3 and all downstream signaling at all time points examined. FIG. 1E: Cartoon schematic of the EGFR family kinase domain asymmetric dimer. The C-terminal domain of the ‘activator’ kinase (right) interacts with the N-terminal portion of the ‘receiver’ kinase (left). This interaction stabilizes the active conformation of the receiver kinase identified by the ‘in’ conformation of the receiver kinase's α-C helix and the ordered extension of the activation loop. The activator kinase retains the inactive conformation.

FIGS. 2A-2D. Lapatinib is unable to bind to the active HER2/HER3 heterodimer. FIG. 2A: SK-BR-3 or MCF-7 cells were serum starved for 24 h and then either treated with lapatinib alone for 15 min—followed by a 15 min NRG stimulation (15 min pre-treat), or were treated with lapatinib and NRG together for 15 min (simultaneous addition). The reduced efficacy of lapatinib when simultaneously added with NRG indicates a reduced ability to bind active HER2 in HER2/HER3 heterodimers. FIG. 2B: NRG rescues the 72 h proliferation of CW-2 cells, which contain an activating mutation in HER3 (E928G), from HER2 inhibitors (mean±SD, n=3). FIG. 2C: CW-2 cells treated with a dose response of lapatinib in the presence or absence of NRG for 1 h show that NRG rescues HER2/HER3 signaling. FIG. 2D: NCI-H1781 cells were treated with a dose response of lapatinib, and signaling was evaluated after 15 min. The short treatment time shows lapatinib does not efficiently bind to HER2 mutants biased towards the active conformation.

FIGS. 3A-3D. Design and execution of a high-throughput screen identifies a novel HER2/HER3 inhibitor. FIG. 3A: 2YF/3wt cells were incubated in the presence or absence of NRG for 48 h and proliferation was assessed by CellTiter-Glo (mean±SD, n=1). FIG. 3B: 48 h proliferation curves of the Ba/F3 panel show they can separate out compounds that specifically inhibit signaling at the HER2/HER3 level (lapatinib) from those that hit downstream (PIK-93) (mean±SD, for lapatinib n=3, for PIK-93 n=1). FIG. 3C: Structure and proliferation curves for hit compound 1 (also referred herein as compound 185) against the Ba/F3 cell line panel (mean±SD, n=3), FIG. 3D: Structure and proliferation curves for compound 2 (also referred to herein as compound 55A) against the panel of Ba/F3 cell lines (mean±SD, n=3).

FIGS. 4A-4F. Compound 2 is a selective Type I inhibitor for HER2. FIG. 4A: In vitro kinase assay of the HER2 kinase domain against lapatinib and compound 2 (mean±SD, n=3). FIG. 4B: Thermal stabilization of the HER3 kinase domain by either compound 2 or ATP as determined by Thermofluor (mean±SD, n=3). FIGS. 4C-4D: The crystal structure of either erlotinib (FIG. 4C) or 2 (FIG. 4D) bound to EGFR V924R. The kinase domain in complex with compound 2 has been stabilized in the active confirmation by drug binding, despite the mutation—as evidenced by the ordered extension of the activation loop and the inward positioning of the α-C helix. FIG. 4E: Magnified view of the EGFR V924R active site when bound to compound 2 shows the proximity of the β3 lysine (K721) and the glutamate (E738) in the α-C helix, which are positioned so as to make a predicted hydrogen bond. FIG. 4F: Table of IC₅₀ values for NRG stimulated HER2YF/HER3 heterodimers +/−the gatekeeper mutations (mean±SD, n=3). The large shift in potency is only seen when compound 2 is unable to bind to HER2, indicating that its cytotoxicity is due to HER2 inhibition.

FIGS. 5A-5D. A Type 1 inhibitor of HER2 is insensitive to the presence of NRG. FIG. 5A: Chemical structure of compound 3 (also referred to herein as compound 178). FIG. 5B: Proliferation curves for compound 3 against the Ba/F3 cell line panel (mean±SD, n=3). FIG. 5C: 72 h proliferation curves of SK-BR-3 and BT-474 cells treated with a dose response of compound 3 in the presence or absence of NRG indicates that compound 3 is insensitive to the presence of NRG in HER2 overexpressing cell lines (mean±SD, n=3). FIG. 5D: The same assay in FIG. 2A was performed with compound 3 in SK-BR-3 cells.

FIGS. 6A-6H. Compound 3 inhibits the active HER2/HER3 heterodimer in multiple oncogenic settings. FIG. 6A: 72 h proliferation of CW-2 cells against compound 3 in the presence or absence of NRG (mean±SD, n=3). FIG. 6B: CW-2 cells treated with a dose response of compound 3 in the presence or absence of NRG for 1 h. FIG. 6C: 72 h proliferation of NCI-H1781 cells shows that they are sensitive to compound 3 but not to DFG in/α-C out inhibitors (mean±SD, n=3), FIG. 6D: NCI-H1781 cells were treated with a dose response of 3 and signaling was evaluated after 15 min. FIG. 6E: 72 h proliferation curves of CHL-1 cells treated with the indicated inhibitors (mean±SD, n=3). FIG. 6F: The growth of CHL-1 cells treated with a dose response of either lapatinib (left) or compound 3 (right) was monitored over 96 h using the IncuCyte Zoom. Confluence measurements show that compound 3 is more effective at reducing the growth of CHL-1 cells (mean±SD, n=2). FIG. 6G: HER2/HER3 signaling was evaluated in CHL-1 cells treated with a dose response of either lapatinib or compound 3 for 24 h. Compound 3 is better able to inhibit p-HER3 and thus the PI3K/Akt pathway. FIG. 6H: CHL-1 cells were treated with either DMSO or 500 nM lapatinib for 24 h. The cells were then washed and treated with a dose response of either lapatinib or compound 3 for an additional 24 h. Signaling shows compound 3 is better able to inhibit feedback activated HER2/HER3 signaling in CHL-1 cells.

FIGS. 7A-7C. FIGS. 7A-7B: Compound 3 was biased towards inhibition of the mutationally activated forms of EGFR in HCC 827 and NCI-H1650 cells, with near complete inhibition of multiple phosphosites on EGFR at less than 10 nM, while leaving the same phosphosites on wt EGFR relatively unaffected up to 1 μM. Consistent with the resistance seen in the HER2 gatekeeper mutant Ba/F3 cells, mutation of the EGFR gatekeeper to methionine in NCI-H1975 cells caused a shift in the ability of compound 3 to inhibit both signaling and proliferation, although the p-845 site showed inhibition at concentrations less than 1 μM, making compound 3 one of the more potent selective and reversible inhibitors of this mutant in a cell line to be reported. The preferential inhibition of the mutationally activated EGFR by compound 3 was also seen in proliferation assays and is in stark contrast to lapatinib's profile against these cells (FIG. 7C).

FIG. 8. SK-BR-3 cells were treated with DMSO or 1 μM lapatinib in the presence or absence of NRG for 1 h. HER3 is the only member of the EGFR family who remains activated in the presence of both NRG and lapatinib. HER4 was undetectable in this cell line.

FIG. 9. NRG pre-treatment rescues HER2/HER3 signaling from lapatinib. SK-BR-3 cells were serum starved for 24 h and then either treated with NRG or vehicle for 15 min followed by a dose response of lapatinib for 15 min and signaling was analyzed by western blot.

FIG. 10. 48 h proliferation curves of the 2YF/3wt Ba/F3 cell line in the presence of either NRG or IL-3 (mean±SD, n=1).

FIG. 11. Compound 2 binds to the active site of HER3. 1 μM of The HER3 intracellular domain (665-1323) was concentrated on vesicles and incubated with 200 μM ATP in the presence of varying concentrations of compound 2. HER3 kinase activity inhibition was evaluated by western blotting for autophosphorylation of HER3.

FIGS. 12A-12B. Mutation of the gatekeeper residue of HER2 or HER3 to Methionine reduces the binding affinity of compound 2. FIG. 12A. HEK-293T cells were transfected with either wt HER2 or HER2 T798M, which were then treated with a dose response of lapatinib or compound 2 (1 nM-10 μM). FIG. 12B. Stabilization of either wt or T787M HER3 kinase domain by compound 2 compared to DMSO as determined by thermofluor (mean value±SD, n=2).

FIG. 13. In vitro kinase assay of the HER2 kinase domain against compound 3 (mean±SD, n=3).

FIG. 14. SK-BR-3 cells treated with a dose response of compound 3 in the presence or absence of NRG for 1 h.

FIGS. 15A-15C. NRG rescues HER2 overexpressing cell lines from type 1.5 inhibitors but not compound 3. FIG. 15A. BT-474 cells were treated with the indicated concentrations of drugs in the presence or absence of NRG for 72 h. Cell death was determined using CellTox green with the Incucyte Zoom data are represented as mean values±SD (n=3). FIG. 15B. Same experiment as in FIG. 15A, but with SK-BR-3 cells. c. Cells were treated with 1 μM of the indicated inhibitor in the presence of varying concentrations of NRG. The lapatininb data is reproduced from FIG. 1C (mean value±SD, n=3).

FIG. 16. SK-BR-3 cells were treated with NRG +/−1 uM of compound 3 for 1 h. HER3 was purified by immunoprecipitation and analyzed for the presence of HER2.

FIG. 17. MCF-7 cells were serum starved for 24 h and then either treated with compound 3 for 15 min followed by a 15 min NRG stimulation (15 min pre-treat), or compound 3 and NRG were added simultaneously for 15 min (simultaneous addition). Compound 3 shows little to no shift in its ability to inhibit signaling +/−pre-incubation indicating it can bind to the actively signaling HER2/HER3 heterodimer.

FIG. 18. SK-BR-3 cells were serum starved for 24 h and then either treated with NRG or vehicle for 15 min followed by a dose response of compound 3 for 15 min and signaling was analyzed by western blot.

FIGS. 19A-19D. NRG rescues HER3 mutant driven Ba/F3 cells. 48 h proliferation of 2YF/HER3E928G (2YF/3EG) Ba/F3 cells treated with a dose response of (FIG. 19A) lapatinib, (FIG. 19B) TAK-285, or (FIG. 19C) compound 3 in the presence or absence of NRG. The large shift in the ability to inhibit proliferation by the current HER2 drugs shows that HER3 mutants could be rescued from the effects of HER2 drugs by NRG in a manner similar to HER2 over expressing cells (mean value±SD, n=3). FIG. 19D. Table of IC50 values for the 2YF/3EG cell lines (nM±SD, n=3).

FIGS. 20A-20B. FaDu cells are more sensitive to compound 3 compared to lapatinib. FIG. 20A. 72 h Proliferation of FaDu cells shows compound 3 is more effective than current HER2 inhibitors (mean value±SD, n=3). FIG. 20B. HER2/HER3 signaling was evaluated in FaDu cells treated with a dose response of either lapatinib or compound 3 for 24 h. Compound 3 is better able to inhibit pHER3 and its downstream signaling pathways.

FIG. 21. Pharmacokinetics of compound 3. Plasma concentration of compound 3 following a single administration of 2 mg/kg by IV or IP. Also shown are the pharmacokinetic (PK) parameters of compound 3.

FIGS. 22A-22H. Screening assays of compounds 184 (FIG. 22A), 185 (FIG. 22B), 189A (FIG. 22C), 189B (FIG. 22D), 190A (FIG. 22E), 190C (FIG. 22F), 190D (FIG. 22G), and 191A (FIG. 22H).

FIGS. 23A-23H. Screening assays of compounds 191B (FIG. 23A), 191D (FIG. 23B), 191E (FIG. 23C), 191F (FIG. 23D), 191H (FIG. 23E), 5-001A (FIG. 23F), 5-001B (FIG. 23G), and 5-004 (FIG. 23H).

FIGS. 24A-24H. Screening assays of compounds 6 (FIG. 24A), 13 (FIG. 24B), 39A (FIG. 24C), 39B (FIG. 24D), 39C (FIG. 24E), 39D (FIG. 24F), 41A (FIG. 24G), and 41B (FIG. 24H).

FIGS. 25A-25H. Screening assays of compounds 42 (FIG. 25A), 43 (FIG. 25B), 45A (FIG. 25C), 45B (FIG. 25D), 45C (FIG. 25E), 45D (FIG. 25F), 45E (FIG. 25G), and 45F (FIG. 25H).

FIGS. 26A-26D. Screening assays of compounds 53B (FIG. 26A), 55A (FIG. 26B), 57A (FIG. 26C), and 57B (FIG. 26D).

FIGS. 27A-27D. Screening assays of compounds 65 (FIG. 27A), 66A (FIG. 27B), 66B (FIG. 27C), and 66C (FIG. 27D).

FIGS. 28A-28H. Screening assays of compounds 144A (FIG. 28A), 144B (FIG. 28B), 147 (FIG. 28C), 152 (FIG. 28D), 153 (FIG. 28E), 154A (FIG. 28F), 154B (FIG. 28G), and 154C (FIG. 28H).

FIGS. 29A-29D. Screening assays of compounds 170 (FIG. 29A), 171 (FIG. 29B), 172 (FIG. 29C), and 173B (FIG. 29D).

FIGS. 30A-30B. Screening assays of compounds 178 (FIG. 30A) and 176 (FIG. 30B).

FIGS. 31A-31D. Screening assays of compounds CJN-08-089 (FIG. 31A), CJN-08-090 (FIG. 31B), CJN-08-091 (FIG. 31C), and CJN-08-092 (FIG. 31D). Compounds CJN-08-089, CJN-08-090, CJN-08-091, and CJN-08-092 may also be referred to herein as 8-089, 8-090, 8-091, and 8-092 respectively. Compounds CJN-08-089, CJN-08-090, CJN-08-091, and CJN-08-092 may also be referred to herein as 8089, 8090, 8091, and 8092 respectively

FIGS. 32A-32D. Screening assays of compounds 89 (FIG. 32A), 90 (FIG. 32B), 91 (FIG. 32C), and 92 (FIG. 32D).

FIGS. 33A-33C. Screening assays and nonlinear fits of compounds CJN-08-095 (FIG. 33A), CJN-08-096 (FIG. 33B), and CJN-08-097 (FIG. 33C). Compounds CJN-08-095, CJN-08-096, and CJN-08-097 may also be referred to herein as 08-095, 08-096, and 08-097. Compounds CJN-08-095, CJN-08-096, and CJN-08-097 may also be referred to herein as 8095, 8096, and 8097.

FIGS. 34A-34C. Screening assays of compounds 95 (FIG. 34A), 96 (FIG. 34B), and 97 (FIG. 34C).

FIGS. 35A-35B. Screening assays and nonlinear fits of compounds CJN-08-104A (FIG. 35A) and CJN-08-104B (FIG. 35B).

FIG. 37. Average IC50 assay results from 6 compounds, 45A, 45E, 53B, 55A, 57A, and 57B, compared to lapatinib.

FIGS. 38A-38B. Average IC50 assay results from 9 compounds, 45E, 57A, 57B, 144A, 144B, 147, 153, 154B, and 155, over a wide range 0 to 1500 nM (FIG. 38A) and over a restricted range 0-300 nM (FIG. 38B).

FIG. 39. Summary of average IC50 assay results in various cell lines for the following compounds: 178, 8096, 8134, 8164, 8168A, 8168B, 8168C, 8177, 8179, 8184, 8168 dasatinib, sapitinib, XL-880, and 8185. For the analysis of human cell line proliferation and determination of the IC50, the indicated cell lines were first transduced with a lentivirus encoding a nuclear localized mRuby. The cells were selected with puro for 4 days and were then routinely passaged in media containing ¼ the amount of puro used for the selection. The cells were then seeded in clear bottom black 384 well plates and allowed to attach to the plates for 24 h in media with no puro. After 24 h media containing drug or drug+growth factor (if indicated) was added on top (1:1 seed media to new media) and the cell growth was monitored for 72 h using the incucyte zoom in both the phase and fluorescent channel (384 whole well scan). The Essen software was then used to analyze the images and count the number of cells/well using the fluorescent nuclei. After 72 h the counts were exported to excel and the number of cells/well was normalized to time 0. These values were then entered into prism (technical triplicate) and used to calculate the area under the curve. This AUC was normalized to the DMSO control on the plate. The process was repeated either 1 or 2 times depending on the compound and cell condition, the averages of these biological triplicate or duplicate values were then analyzed to determine the IC50.

FIG. 40. The cell viability across different cell lines for compound 8156. Compound 8156 has a MW of 428.44, Log P of 3; tPSA of 116.54, and a C Log P of 3.48644.

### DETAILED DESCRIPTION

Herein we identify novel HER2/HER3 inhibitors that preferentially target the active state of the heterodimer. An inhibitor described herein is capable of potently inhibiting signaling from the HER2/HER3 heterodimer regardless of the activating oncogenic mechanism.

A. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched non-cyclic chain, or combinations thereof, including at least one carbon atom and at least one heteroatom (e.g., selected from the group consisting of O, N, P, Si, and S, and wherein the nitrogen and sulfur atoms may optionally be oxidized, and the nitrogen heteroatom may optionally be quaternized). The heteroatom(s) (e.g., O, N, P, S, and Si) may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)N R′R″, —NR″C(O)R′, —NR⁷—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, NR′NR″R″′, ONR′R″, —NR′C═(O)NR″NR″′R″″, —CN, —NO₂, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC (O)NR′R″, —NR″C(O)R′, —NR⁷—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, NR′NR″R″′, ONR′R″, NR′C═(O)NR″NR″′R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R″′, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR⁷—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R″′)_{d}—, where s and d are independently integers of from 0 to 3, and X′ is —O—, —S—, —S(O)—, —S(O)₂—, or —S(O)₂NR⁷—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Provided herein are agents (e.g. compounds, drugs, therapeutic agents) that may be in a prodrug form. Prodrugs of the compounds described herein are those compounds that readily undergo chemical changes under select physiological conditions to provide the final agents (e.g. compounds, drugs, therapeutic agents). Additionally, prodrugs can be converted to agents (e.g. compounds, drugs, therapeutic agents) by chemical or biochemical methods in an ex vivo environment. Prodrugs described herein include compounds that readily undergo chemical changes under select physiological conditions to provide agents (e.g. compounds, drugs, therapeutic agents) to a biological system (e.g. in a subject, in a cancer cell, in the extracellular space near a cancer cell).

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat diseases associated with ErbB/HER (e.g., EGFR, HER2, HER3, or HER4) activity. Certain methods described herein may treat diseases associated with ErbB/HER (e.g., EGFR, HER2, HER3, or HER4) activity (e.g., cancer) by inhibiting ErbB/HER (e.g., EGFR, HER2, HER3, or HER4) activity. For example, certain methods herein treat cancer. For example certain methods herein treat cancer by decreasing a symptom of cancer. Symptoms of cancer would be known or may be determined by a person of ordinary skill in the art. The term “treating” and conjugations thereof, include prevention of an injury, pathology, condition, or disease. In embodiments, treating does not include preventing.

An “effective amount” is an amount sufficient to accomplish a stated purpose (e.g. achieve the effect for which it is administered, treat a disease, reduce enzyme activity, increase enzyme activity, reduce protein function, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug or prodrug is an amount of a drug or prodrug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. cancer) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, a disease associated with ErbB/HER (e.g., EGFR, HER2, HER3, or HER4) activity may be treated with an agent (e.g. compound as described herein) effective for decreasing the level of ErbB/HER (e.g., EGFR, HER2, HER3, or HER4) activity.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein (e.g., ErbB/HER (e.g., EGFR, HER2, HER3, or HER4)) or enzyme.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein.

The term “modulator” refers to a composition that increases or decreases the level of a target molecule or the function of a target molecule relative to a standard control (e.g., such as the absence of the modulator). In embodiments, a modulator is an anti-cancer agent. In embodiments, a modulator is an ErbB/HER (e.g., EGFR, HER2, HER3, or HER4) antagonist. In embodiments, a modulator is an ErbB/HER (e.g., EGFR, HER2, HER3, or HER4) agonist.

“Anti-cancer agent” or “anti-cancer drug” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent approved by the FDA or similar regulatory agency of a country other than the USA, for treating cancer. Examples of anti-cancer agents include, but are not limited to, anti-androgens (e.g., Casodex, Flutamide, MDV3100, or ARN-509), MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone;leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gurin (BCG), levamisole, interleukin-2, alpha-interferon, etc.), monoclonal antibodies (e. g. , anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g. tomaymycin), carboplatin, CC-1065 and CC-1065 analogs including amino-CBIs, nitrogen mustards (such as chlorambucil and melphalan), dolastatin and dolastatin analogs (including auristatins: eg. monomethyl auristatin E), anthracycline antibiotics (such as doxorubicin, daunorubicin, etc.), duocarmycins and duocarmycin analogs, enediynes (such as neocarzinostatin and calicheamicins), leptomycin derivatives, maytansinoids and maytansinoid analogs (e.g. mertansine), methotrexate, mitomycin C, taxoids, vinca alkaloids (such as vinblastine and vincristine), epothilones (e.g. epothilone B), camptothecin and its clinical analogs topotecan and irinotecan, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

“Patient” or “subject in need thereof” or “subject” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In some embodiments, a patient is human. In some embodiments, a subject is human.

“Disease” or “condition” refer to a state of being or health status of a patient or subject capable of being treated with a compound, pharmaceutical composition, or method provided herein. In some embodiments, the disease is a disease having the symptom of cell hyperproliferation. In some embodiments, the disease is a disease having the symptom of an aberrant level of ErbB/HER (e.g., EGFR, HER2, HER3, or HER4) activity. In some embodiments, the disease is a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma. In embodiments, the disease is brain cancer. In embodiments, the disease is neuroblastoma. In embodiments, the disease is glioblastoma.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include cancer of the prostate, thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples may include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. Substitution of one amino acid residue for another from a different class or group is referred to herein as a “non-conservative” substitution. In contrast, non-conservative amino acid substitutions tend to modify conformation and function of a protein. Example of amino acid classification:

Small/Aliphatic residues: Gly, Ala, Val, Leu, Ile

Cyclic Imino Acid: Pro

Hydroxyl-containing Residues: Ser, Thr

Acidic Residues: Asp, Glu

Amide Residues: Asn, Gln

Basic Residues: Lys, Arg

Imidazole Residue: His

Aromatic Residues: Phe, Tyr, Trp

Sulfur-containing Residues: Met, Cys

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (O) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., BIOCHEMISTRY at pp. 13-15, 2nd ed. Lubert Stryer ed. (Stanford University); Henikoff et al., Proc. Nat'l Acad. Sci. USA (1992) 89:10915-10919; Lei et al., J. Biol. Chem. (1995) 270(20):11882-11886).

“Polypeptide,” “peptide,” and “protein” are used herein interchangeably and mean any peptide-linked chain of amino acids, regardless of length or post-translational modification. As noted below, the polypeptides described herein can be, e.g., wild-type proteins, biologically-active fragments of the wild-type proteins, or variants of the wild-type proteins or fragments. Variants, in accordance with the disclosure, can contain amino acid substitutions, deletions, or insertions. The substitutions can be conservative or non-conservative.

Following expression, the proteins can be isolated. The term “purified” or “isolated” as applied to any of the proteins described herein refers to a polypeptide that has been separated or purified from components (e.g., proteins or other naturally-occurring biological or organic molecules) which naturally accompany it, e.g., other proteins, lipids, and nucleic acid in a cell expressing the proteins. Typically, a polypeptide is purified when it constitutes at least 60 (e.g., at least 65, 70, 75, 80, 85, 90, 92, 95, 97, or 99) %, by weight, of the total protein in a sample.

An amino acid residue in a protein “corresponds” to a given residue when it occupies the same essential structural position within the protein as the given residue. For example, a selected residue in a selected protein corresponds to a particular amino acid in an ErbB/HER (e.g., EGFR, HER2, HER3, or HER4) when the selected residue occupies the same essential spatial or other structural relationship as particular amino acid in an ErbB/HER (e.g., EGFR, HER2, HER3, or HER4). In some embodiments, where a selected protein is aligned for maximum homology with the human ErbB/HER (e.g., EGFR, HER2, HER3, or HER4) protein, the position in the aligned selected protein aligning with a particular reside is said to correspond to that particular reside. Instead of a primary sequence alignment, a three dimensional structural alignment can also be used, e.g., where the structure of the selected protein is aligned for maximum correspondence with the human ErbB/HER (e.g., EGFR, HER2, HER3, or HER4) protein and the overall structures compared. In this case, an amino acid that occupies the same essential position as a particular reside in the structural model is said to correspond to the particular reside.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Pharmaceutical compositions provided by the present invention include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms (e.g. symptoms of cancer or aberrant ErbB/HER (e.g., EGFR, HER2, HER3, or HER4) activity). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer such as radiation or surgery.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “Receptor tyrosine-protein kinase erbB-3”, “human epidermal growth factor receptor 3 “ERBB3”, or “HER3” refers to a pseudokinase (reduced activity or inactive kinase) that is a member of the epidermal growth factor receptor (EGFR/ERBB) family of receptor tyrosine kinases. The term “HER3” may refer to the nucleotide sequence or protein sequence of human HER3 (e.g., Entrez 2065, Uniprot P21860, RefSeq NM_001982, or RefSeq NP_001973) and homologs thereof. The term “HER3” includes both the wild-type form of the nucleotide sequences or proteins as well as any mutants thereof. In some embodiments, “HER3” is wild-type HER3 receptor. In some embodiments, “HER3” is one or more mutant forms. The term “HER3” XYZ refers to a nucleotide sequence or protein of a mutant HER3wherein the Y numbered amino acid of HER3 that normally has an X amino acid in the wildtype, instead has a Z amino acid in the mutant. In embodiments, an HER3 is the human HER3. In embodiments, the HER3 has the nucleotide sequence corresponding to reference number GI:317171925. In embodiments, the HER3 has the nucleotide sequence corresponding to RefSeq NM_001982.3. In embodiments, the HER3 has the protein sequence corresponding to reference number GI:54792100. In embodiments, the HER3 has the protein sequence corresponding to RefSeq NP_001973.2. In embodiments, the HER3 has the following amino acid sequence:

(SEQ ID NO: 1)
MRANDALQVLGLLFSLARGSEVGNSQAVCPGTLNGLSVTGDAENQYQTLY
KLYERCEVVMGNLEIVLTGHNADLSFLQWIREVTGYVLVAMNEFSTLPLP
NLRVVRGTQVYDGKFAIFVMLNYNTNSSHALRQLRLTQLTEILSGGVYIE
KNDKLCHMDTIDWRDIVRDRDAEIVVKDNGRSCPPCHEVCKGRCWGPGSE
DCQTLTKTICAPQCNGHCFGPNPNQCCHDECAGGCSGPQDTDCFACRHFN
DSGACVPRCPQPLVYNKLTFQLEPNPHTKYQYGGVCVASCPHNFVVDQTS
CVRACPPDKMEVDKNGLKMCEPCGGLCPKACEGTGSGSRFQTVDSSNIDG
FVNCTKILGNLDFLITGLNGDPWHKIPALDPEKLNVFRTVREITGYLNIQ
SWPPHMHNFSVFSNLTTIGGRSLYNRGFSLLIMKNLNVTSLGFRSLKEIS
AGRIYISANRQLCYHHSLNWTKVLRGPTEERLDIKHNRPRRDCVAEGKVC
DPLCSSGGCWGPGPGQCLSCRNYSRGGVCVTHCNFLNGEPREFAHEAECF
SCHPECQPMEGTATCNGSGSDTCAQCAHFRDGPHCVSSCPHGVLGAKGPI
YKYPDVQNECRPCHENCTQGCKGPELQDCLGQTLVLIGKTHLTMALTVIA
GLVVIFMMLGGTFLYWRGRRIQNKRAMRRYLERGESIEPLDPSEKANKVL
ARIFKETELRKLKVLGSGVFGTVHKGVWIPEGESIKIPVCIKVIEDKSGR
QSFQAVTDHMLAIGSLDHAHIVRLLGLCPGSSLQLVTQYLPLGSLLDHVR
QHRGALGPQLLLNWGVQIAKGMYYLEEHGMVHRNLAARNVLLKSPSQVQV
ADFGVADLLPPDDKQLLYSEAKTPIKWMALESIHFGKYTHQSDVWSYGVT
VWELMTFGAEPYAGLRLAEVPDLLEKGERLAQPQICTIDVYMVMVKCWMI
DENIRPTFKELANEFTRMARDPPRYLVIKRESGPGIAPGPEPHGLTNKKL
EEVELEPELDLDLDLEAEEDNLATTTLGSALSLPVGTLNRPRGSQSLLSP
SSGYMPMNQGNLGESCQESAVSGSSERCPRPVSLHPMPRGCLASESSEGH
VTGSEAELQEKVSMCRSRSRSRSPRPRGDSAYHSQRHSLLTPVTPLSPPG
LEEEDVNGYVMPDTHLKGTPSSREGTLSSVGLSSVLGTEEEDEDEEYEYM
NRRRRHSPPHPPRPSSLEELGYEYMDVGSDLSASLGSTQSCPLHPVPIMP
TAGTTPDEDYEYMNRQRDGGGPGGDYAAMGACPASEQGYEEMRAFQGPGH
QAPHVHYARLKTLRSLEATDSAFDNPDYWHSRLFPKANAQRT

In embodiments, the HER3 is a mutant HER3. In embodiments, the mutant HER3 is associated with a disease that is not associated with wildtype HER3. In embodiments, the HER3 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above. In embodiments, the HER3 is a variant of the sequence above, including a shorter variant or mutated variant. In embodiments, the mutant HER3 is a splice variant. In embodiments, the mutant HER3 is a splice variant with aberrant activity relative to the widtype HER3. In embodiments, the mutant HER3 is a truncated splice variant with aberrant activity relative to the widtype HER3. In embodiments, the mutant HER3 is a splice variant lacking a portion of the wildtype HER3 with aberrant activity relative to the widtype HER3. In embodiments, the HER3 is described in Cancer Cell (2013) May 13 23, 603-617, which is herein incorporated in its entirety for all purposes.

The term “Receptor tyrosine-protein kinase erbB-2”, “human epidermal growth factor receptor 2”, “CD340”, “ERBB2”, “neu”, “HER2/neu”, or “HER2” refers to a member of the epidermal growth factor receptor (EGFR/ERBB) family of receptor tyrosine kinases. The term “HER2” may refer to the nucleotide sequence or protein sequence of human HER2 (e.g., Entrez 2064, Uniprot P04626, RefSeq NM_004448, or RefSeq NP_004439) and homologs thereof. The term “HER2” includes both the wild-type form of the nucleotide sequences or proteins as well as any mutants thereof. In some embodiments, “HER2” is wild-type HER2 receptor. In some embodiments, “HER2” is one or more mutant forms. The term “HER2” XYZ refers to a nucleotide sequence or protein of a mutant HER2 wherein the Y numbered amino acid of HER2 that normally has an X amino acid in the wildtype, instead has a Z amino acid in the mutant. In embodiments, an HER2 is the human HER2. In embodiments, the HER2 has the nucleotide sequence corresponding to reference number GI:584277099. In embodiments, the HER2 has the nucleotide sequence corresponding to RefSeq NM_004448.3. In embodiments, the HER2 has the protein sequence corresponding to reference number GI:54792096. In embodiments, the HER2 has the protein sequence corresponding to RefSeq NP_004439.2. In embodiments, the HER2 has the following amino acid sequence:

(SEQ ID NO: 2)
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

In embodiments, the HER2 is a mutant HER2. In embodiments, the mutant HER2 is associated with a disease that is not associated with wildtype HER2. In embodiments, the HER2 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above. In embodiments, the HER2 is a variant of the sequence above, including a shorter variant or mutated variant. In embodiments, the mutant HER2 is a splice variant. In embodiments, the mutant HER2 is a splice variant with aberrant activity relative to the widtype HER2. In embodiments, the mutant HER2 is a truncated splice variant with aberrant activity relative to the widtype HER2. In embodiments, the mutant HER2 is a splice variant lacking a portion of the wildtype HER2 with aberrant activity relative to the widtype HER2.

The term “epidermal growth factor receptor”, “ErbB1”, and “EGFR” refer to a member of the epidermal growth factor receptor (EGFR/ERBB) family of receptor tyrosine kinases. The term “EGFR” may refer to the nucleotide sequence or protein sequence of human EGFR (e.g., Entrez 1956, Uniprot P00533, RefSeq NM_05228, or RefSeq NP_005219) and homologs thereof. The term “EGFR” includes both the wild-type form of the nucleotide sequences or proteins as well as any mutants thereof. In some embodiments, “EGFR” is wild-type EGFR receptor. In some embodiments, “EGFR” is one or more mutant forms. The term “EGFR” XYZ refers to a nucleotide sequence or protein of a mutant EGFR wherein the Y numbered amino acid of EGFR that normally has an X amino acid in the wildtype, instead has a Z amino acid in the mutant. In embodiments, an EGFR is the human EGFR. In embodiments, the EGFR has the nucleotide sequence corresponding to reference number GI:41327737. In embodiments, the EGFR has the nucleotide sequence corresponding to RefSeq NM_005228.3. In embodiments, the EGFR has the protein sequence corresponding to reference number GI:29725609. In embodiments, the EGFR has the protein sequence corresponding to RefSeq NP_005219.2. In embodiments, the EGFR has the following amino acid sequence:

(SEQ ID NO: 3)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSFAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA

In embodiments, the EGFR is a mutant EGFR (e.g., exon 20 mutant). In embodiments, the mutant EGFR is associated with a disease that is not associated with wildtype EGFR. In embodiments, the EGFR includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above. In embodiments, the EGFR is a variant of the seqeuence above, including a shorter variant or mutated variant. In embodiments, the mutant EGFR is a splice variant. In embodiments, the mutant EGFR is a splice variant with aberrant activity relative to the widtype EGFR. In embodiments, the mutant EGFR is a truncated splice variant with aberrant activity relative to the widtype EGFR. In embodiments, the mutant EGFR is a splice variant lacking a portion of the wildtype EGFR with aberrant activity relative to the widtype EGFR.

The term “ligand” is used in accordance with its plain ordinary meaning and refers to a molecule (e.g., compound as described herein) capable of binding to another molecule (e.g., protein, receptor, enzyme, target, or cell). In embodiments, a ligand is a modulator, inhibitor, activator, agonist, or antagonist.

The terms “analog” or “analogue” are used in accordance with their plain ordinary meaning in Chemistry and refers to a compound having a structure (e.g., chemical structure) similar to another compound (reference compound, compound described herein) but differing in one or more components (e.g., different substituent(s), addition of substituent(s), removal of substituent(s)).

The term “derivative” is used in accordance with its plain ordinary meaning in chemistry and refers to a compound that is derived (e.g., a product made from a reactant) from a similar compound by a chemical or physical process.

The term “HER2 activity” is used in accordance with its plain ordinary meaning and refers to the function or activity of the HER2 protein. Examples of HER2 activity include dimerization (e.g., heterodimerization). In embodiments, HER2 activity is increasing or activating activity of a protein interacting with HER2 (e.g., PI3K activity, MEK activity, MAPK activity, RAF activity, BRAF activity, AKT activity, RAS activity, or KRAS activity). In embodiments HER2 activity is activation or increasing of activity of a signaling pathway by HER2 or activation of a component of a signaling pathway by HER2 (e.g., directly or through intervening components of the signaling pathway). In embodiments HER2 activity is activation of kinase activity of a protein that interacts (e.g., directly contacting HER3 or interactions with HER2 through intermediates) with HER2 (e.g., EGFR, HER3, HER4, c-MET, PI3K, MEK, MAPK, RAF, BRAF, AKT, RAS, or KRAS).

The term “EGFR activity” is used in accordance with its plain ordinary meaning and refers to the function or activity of the EGFR protein. Examples of EGFR activity include dimerization (e.g., heterodimerization) or activation of the activity of a protein upon dimerization of EGFR (e.g., HER2 activity, HER3 activity, HER4 activity, or c-MET activity). In embodiments, EGFR activity is increasing or activating activity of a protein interacting with EGFR (e.g., PI3K activity, MEK activity, MAPK activity, RAF activity, BRAF activity, AKT activity, RAS activity, or KRAS activity). In embodiments EGFR activity is activation or increasing of activity of a signaling pathway by EGFR or activation of a component of a signaling pathway by EGFR (e.g., directly or through intervening components of the signaling pathway). In embodiments HER2 activity is activation of kinase activity of a protein that interacts (e.g., directly contacting EGFR or interactions with EGFR through intermediates) with EGFR (e.g., HER2, HER3, HER4, c-MET, PI3K, MEK, MAPK, RAF, BRAF, AKT, RAS, or KRAS).

The term “active conformation” when referring to the ERBB (e.g., HER2 or EGFR) protein, is the protein conformation in which the protein has kinase activity (e.g., an increased kinase activity relative to an inactive conformation or basal activity when not activated (e.g., by ligand binding or dimerization)). The active conformation may be characterized by, for example, the “in conformation” of the α-C helix, which allows formation of the characteristic salt-bridge between a β3 lysine and an α-C glutamate or formation of an ordered extension of the activation loop (e.g., in HER2) or a correspondingly similar conformational change in EGFR, HER2, HER3, or HER4.

A “covalent cysteine modifier moiety” as used herein refers to a substituent that is capable of reacting with the sulfhydryl functional group of a cysteine amino acid (e.g. cysteine 797 of human EGFR or cysteine 805 of human HER2) to form a covalent bond. Thus, the covalent cysteine modifier moiety is typically electrophilic.

B. Compounds

In an aspect is provided a compound having the formula:

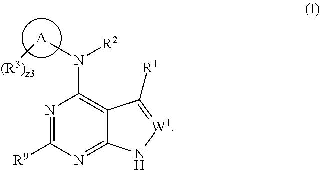

Ring A is aryl or heteroaryl. W¹ is N or C(H). R¹ is hydrogen, -L¹-L²-E, substituted or unsubstituted aryl, or substituted or unsubstituted heteroaryl. R² is hydrogen, —CX²₃, —CHX²₂, —CH₂X², substituted or unsubstituted alkyl, or substituted or unsubstituted heteroalkyl. R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—R^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol z3 is an integer from 0 to 4. L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁶ is hydrogen, halogen, —CX⁶₃, —CHX⁶₂, —CH₂X⁶, —OCX⁶₃, —OCH₂X⁶, —OCHX⁶₂, —CN, SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. E is an electrophilic moiety. Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl. R^{9A} and R^{9B} substituents bonded to the same nitrogen atom may optionally be joined to form a substituted or unsubstituted heterocycloalkyl or substituted or unsubstituted heteroaryl. Each X, X³, X⁶, X⁷, and X⁹ is independently —F, —Cl, —Br, or —I. The symbols n3, n6, n7, and n9 are independently an integer from 0 to 4. The symbols m3, m6, m7, m9, v3, v6, v7, and v9 are independently an integer from 1 to 2.

In embodiments, the compound has the formula:

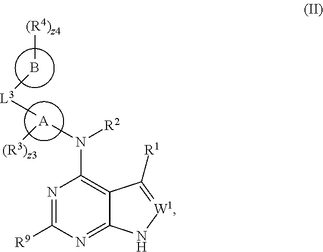

wherein R¹, R², R³, R⁹, Ring A, W¹, and z3 are as described herein.

Ring B is cycloalkyl, heterocycloalkyl, aryl, or heteroaryl. L³ is a bond, —S(O)₂—, —O—, —S—, —C(O)—, —C(O)NR⁸—, —NR⁸C(O)—, —NR⁸C(O)NH—, —NHC(O)NR⁸—, —C(O)O—, —OC(O)—, substituted or unsubstituted alkylene, or substituted or unsubstituted heteroalkylene.

R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is independently hydrogen, halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol z4 is an integer from 0 to 5. Each R^{4A}, R^{4B}, R^{4C}, R^{4D}, R^{8A}, R^{8B}, R^{8C}, and R^{8D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl. Each X⁴ and X⁸ is independently —F, —Cl, —Br, or —I. The symbols n4 and n8 are independently an integer from 0 to 4. The symbols m4, m8, v4, and v8, are independently an integer from 1 to 2.

In embodiments, the compound has the formula:

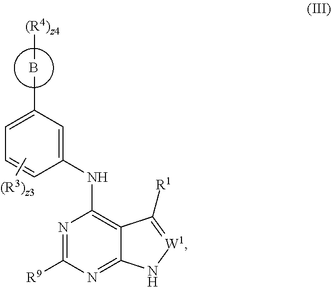

wherein R¹, R², R³, R⁴, R⁹, Ring B, W¹, z3, and z4 are as described herein.

In embodiments, the compound has the formula:

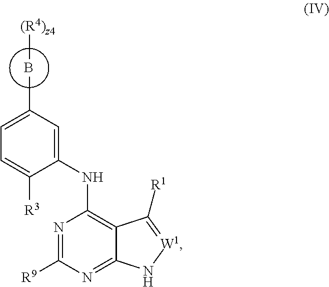

wherein R¹, R³, R⁴, R⁹, Ring B, W¹, and z4 are as described herein.

In embodiments, the compound has the formula:

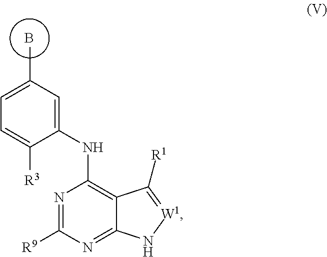

wherein R¹, R³, R⁹, Ring B, and W¹ are as described herein.

In embodiments, the compound has the formula:

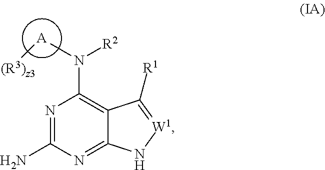

wherein R¹, R², R³, Ring A, W¹, and z3 are as described herein.

In embodiments, the compound has the formula:

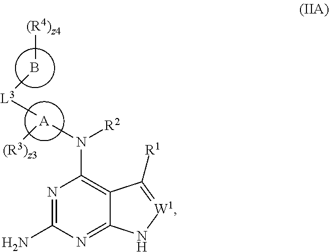

wherein R¹, R², R³, R⁴, L³, Ring A, Ring B, W¹, z3, and z4 are as described herein.

In embodiments, the compound has the formula:

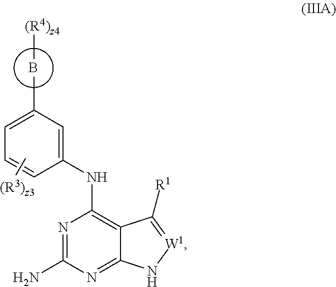

wherein R¹, R³, R⁴, Ring B, W¹, z3, and z4 are as described herein.

In embodiments, the compound has the formula:

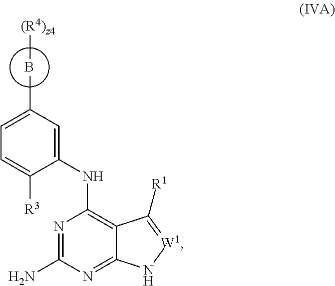

wherein R¹, R³, R⁴, Ring B, W¹, and z4 are as described herein.

In embodiments, the compound has the formula:

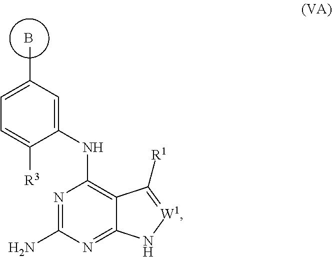

wherein R¹, R³, Ring B, and W¹ are as described herein.

In embodiments, the compound has the formula:

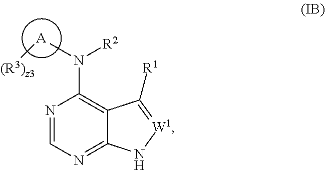

wherein R¹, R², R³, Ring A, W¹, and z3 are as described herein.

In embodiments, the compound has the formula:

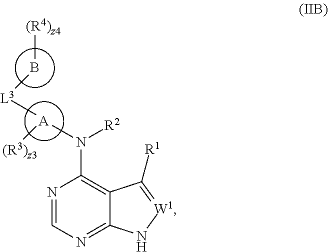

wherein R¹, R², R³, R⁴, L³, Ring A, Ring B, W¹, z3, and z4 are as described herein.

In embodiments, the compound has the formula:

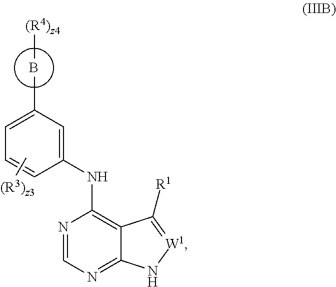

wherein R¹, R³, R⁴, Ring B, W¹, z3, and z4 are as described herein.

In embodiments, the compound has the formula:

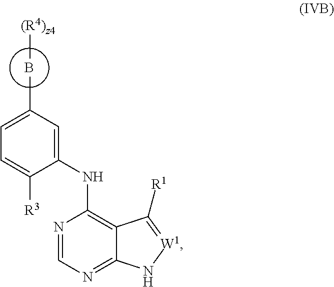

wherein R¹, R³, R⁴, Ring B, W¹, and z4 are as described herein.

In embodiments, the compound has the formula:

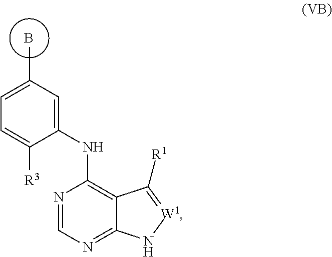

wherein R¹, R³, Ring B, and W¹ are as described herein.

In embodiments, the compound has the formula:

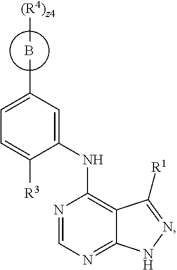

wherein R¹, R³, R⁴, Ring B, and z4 are as described herein.

In embodiments, the compound has the formula:

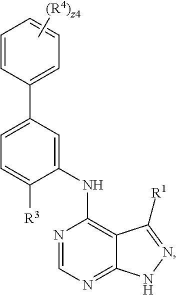

wherein R¹, R³, R⁴, and z4 are as described herein.

In embodiments, the compound has the formula:

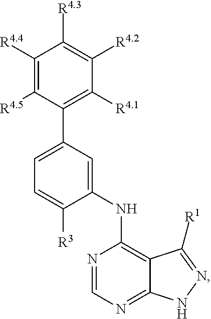

wherein R¹ and R³ are as described herein. R^{4.1}, R^{4.2}, R^{4.3}, R^{4.4}, and R^{4.5} are each independently R⁴ at a fixed position (e.g., non-floating as shown in the formula described herein) and may independently be any R⁴ substituent.

In embodiments, the compound has the formula:

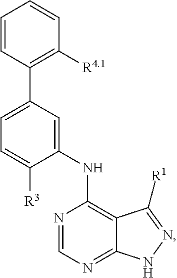

wherein R¹ and R³ are as described herein. R^{4.1} is an R⁴ substituent at a fixed position (e.g., non-floating as shown in the formula described herein) and may independently be any R⁴ substituent.

In embodiments, the compound has the formula:

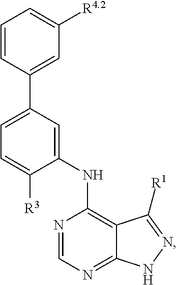

wherein R¹ and R³ are as described herein. R^{4.2} is an R⁴ substituent at a fixed position (e.g., non-floating as shown in the formula described herein) and may independently be any R⁴ substituent.

In embodiments, the compound has the formula:

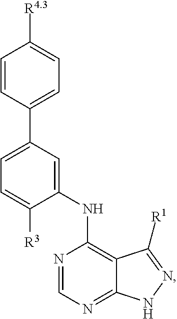

wherein R¹ and R³ are as described herein. R^{4.3} is an R⁴ substituent at a fixed position (e.g., non-floating as shown in the formula described herein) and may independently be any R⁴ substituent.

In embodiments, the compound has the formula:

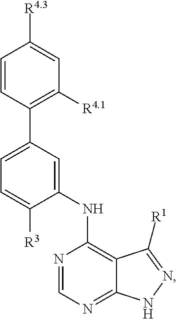

wherein R¹ and R³ are as described herein. R^{4.1} and R^{4.3} are each independently R⁴ at a fixed position (e.g., non-floating as shown in the formula described herein) and may independently be any R⁴ substituent.

In embodiments, the compound has the formula:

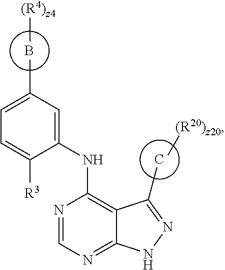

wherein R¹, R³, R⁴, Ring B, R²⁰, and z4 are as described herein. Ring C is a substituted or unsubstituted aryl, or substituted or unsubstituted heteroaryl. The symbol z20 is an integer from 0 to 5.

In embodiments, the compound has the formula:

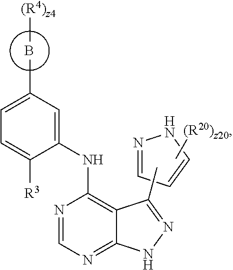

wherein R¹, R³, R⁴, Ring B, R²⁰, z20, and z4 are as described herein. In embodiments, z20 is an integer from 0 to 4. In embodiments, z20 is an integer from 0 to 3. In embodiments, z20 is an integer from 0 to 2.

In embodiments, the compound has the formula:

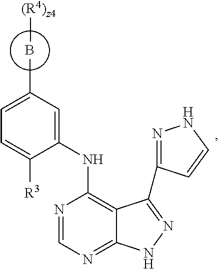

wherein R¹, R³, R⁴, Ring B, and z4 are as described herein.

In embodiments, the compound has the formula:

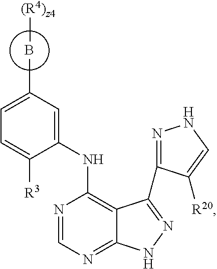

wherein R¹, R³, R⁴, Ring B, and R²⁰ are as described herein.

In embodiments, the compound has the formula:

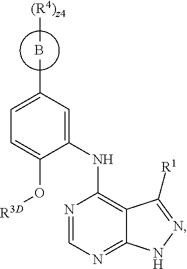

wherein R¹, R^{3D}, R⁴, Ring B, and z4 are as described herein.

In embodiments, the compound has the formula:

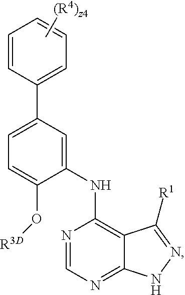

wherein R¹, R³, R⁴, and z4 are as described herein.

In embodiments, W¹ is C(H). In embodiments, W¹ is N.

In embodiments, Ring A is substituted or unsubstituted aryl. In embodiments, Ring A is substituted or unsubstituted heteroaryl. In embodiments, Ring A is substituted or unsubstituted C₆-C₁₀ aryl. In embodiments, Ring A is substituted or unsubstituted C₁₀ aryl. In embodiments, Ring A is substituted or unsubstituted phenyl. In embodiments, Ring A is substituted or unsubstituted 5 to 10 membered heteroaryl. In embodiments, Ring A is substituted or unsubstituted 5 to 9 membered heteroaryl. In embodiments, Ring A is substituted or unsubstituted 5 to 6 membered heteroaryl. In embodiments, Ring A is substituted or unsubstituted 10 membered heteroaryl. In embodiments, Ring A is substituted or unsubstituted 9 membered heteroaryl. In embodiments, Ring A is substituted or unsubstituted 5 membered heteroaryl. In embodiments, Ring A is substituted or unsubstituted 6 membered heteroaryl. In embodiments, Ring A is substituted or unsubstituted phenyl. In embodiments, Ring A is substituted or unsubstituted pyridyl. In embodiments, Ring A is substituted or unsubstituted pyrazolyl. In embodiments, Ring A is substituted or unsubstituted pyrimidyl. In embodiments, Ring A is substituted or unsubstituted imidazolyl. In embodiments, Ring A is substituted or unsubstituted oxazolyl. In embodiments, Ring A is substituted or unsubstituted isoxazolyl. In embodiments, Ring A is substituted or unsubstituted thiazolyl. In embodiments, Ring A is substituted or unsubstituted furanyl. In embodiments, Ring A is substituted or unsubstituted pyrrolyl. In embodiments, Ring A is substituted or unsubstituted thienyl. In embodiments, Ring A is a two fused ring aryl. In embodiments, Ring A is a two fused ring heteroaryl.

In embodiments, Ring A is substituted or unsubstituted aryl or heteroaryl. In embodiments, Ring A is substituted or unsubstituted phenyl or 5 to 6 membered heteroaryl. In embodiments, Ring A is substituted or unsubstituted phenyl. In embodiments, Ring A is substituted or unsubstituted 5 to 6 membered heteroaryl. In embodiments, Ring A is substituted or unsubstituted pyrazinyl, pyrimidinyl, furanyl, thienyl, pyrrolyl, imidazolyl, pyrazolyl, oxazolyl, isoxazolyl, or thiazolyl.

In embodiments, Ring A is substituted or unsubstituted phenyl. In embodiments, Ring A is substituted or unsubstituted pyridyl. In embodiments, Ring A is substituted or unsubstituted cyclohexyl. In embodiments, Ring A is substituted or unsubstituted morpholinyl. In embodiments, Ring A is substituted or unsubstituted piperazinyl. In embodiments, Ring A is substituted or unsubstituted furanyl. In embodiments, Ring A is substituted or unsubstituted thiazolyl. In embodiments, Ring A is substituted or unsubstituted pyrazolyl. In embodiments, Ring A is substituted or unsubstituted thienyl. In embodiments, Ring A is substituted or unsubstituted pyrazinyl. In embodiments, Ring A is substituted or unsubstituted pyrimidinyl. In embodiments, Ring A is substituted or unsubstituted pyridazinyl. In embodiments, Ring A is substituted or unsubstituted triazinyl. In embodiments, Ring A is substituted or unsubstituted tetrazinyl. In embodiments, Ring A is substituted or unsubstituted tetrazolyl. In embodiments, Ring A is substituted or unsubstituted triazolyl. In embodiments, Ring A is substituted or unsubstituted quinolinyl. In embodiments, Ring A is substituted or unsubstituted isoquinolinyl. In embodiments, Ring A is substituted or unsubstituted quinazolinyl. In embodiments, Ring A is substituted or unsubstituted quinoxalinyl. In embodiments, Ring A is substituted or unsubstituted imidazolyl. In embodiments, Ring A is substituted or unsubstituted oxazolyl. In embodiments, Ring A is substituted or unsubstituted isoxazolyl. In embodiments, Ring A is substituted or unsubstituted thiazolyl. In embodiments, Ring A is substituted or unsubstituted piperidinyl. In embodiments, Ring A is substituted or unsubstituted thiomorpholinyl. In embodiments, Ring A is substituted or unsubstituted thianyl. In embodiments, Ring A is substituted or unsubstituted oxanyl. In embodiments, Ring A is substituted or unsubstituted tetrahydropuranyl. In embodiments, Ring A is substituted or unsubstituted dihydropuranyl. In embodiments, Ring A is substituted or unsubstituted dioxanyl. In embodiments, Ring A is substituted or unsubstituted pyrazolyl. In embodiments, Ring A is substituted or unsubstituted pyrrolyl. In embodiments, Ring A is substituted or unsubstituted thienyl. In embodiments, Ring A is substituted or unsubstituted benzofuranyl. In embodiments, Ring A is substituted or unsubstituted indolyl. In embodiments, Ring A is substituted or unsubstituted benzothienyl. In embodiments, Ring A is substituted or unsubstituted benzimidazolyl. In embodiments, Ring A is substituted or unsubstituted isobenzofuranyl. In embodiments, Ring A is substituted or unsubstituted isoindolyl. In embodiments, Ring A is substituted or unsubstituted benzo[c]thienyl. In embodiments, Ring A is substituted or unsubstituted purinyl. In embodiments, Ring A is substituted or unsubstituted indazolyl. In embodiments, Ring A is substituted or unsubstituted benzoxazolyl. In embodiments, Ring A is substituted or unsubstituted benzisoxazolyl. In embodiments, Ring A is substituted or unsubstituted benzothiazolyl. In embodiments, Ring A is substituted or unsubstituted cyclopentyl. In embodiments, Ring A is substituted or unsubstituted cyclobutyl. In embodiments, Ring A is substituted or unsubstituted 2-thienyl. In embodiments, Ring A is substituted or unsubstituted 3-thienyl. In embodiments, Ring A is substituted or unsubstituted 2-furanyl. In embodiments, Ring A is substituted or unsubstituted 3-furanyl. In embodiments, Ring A is substituted or unsubstituted 2-pyridyl. In embodiments, Ring A is substituted or unsubstituted 3-pyridyl. In embodiments, Ring A is substituted or unsubstituted 4-pyridyl. In embodiments, Ring A is substituted or unsubstituted 3-pyrazolyl. In embodiments, Ring A is substituted or unsubstituted 4-pyrazolyl. In embodiments, Ring A is substituted or unsubstituted 5-pyrazolyl. In embodiments, Ring A is substituted or unsubstituted 2-pyrrolyl. In embodiments, Ring A is substituted or unsubstituted 3-pyrrolyl. In embodiments, Ring A is substituted or unsubstituted 2-thiazolyl. In embodiments, Ring A is substituted or unsubstituted 4-thiazolyl. In embodiments, Ring A is substituted or unsubstituted 5-thiazolyl. In embodiments, Ring A is substituted or unsubstituted 2-pyridyl. In embodiments, Ring A is substituted or unsubstituted 3-pyridyl. In embodiments, Ring A is substituted or unsubstituted 4-pyridyl. In embodiments, Ring A is substituted or unsubstituted phenyl.

In embodiments, Ring A is a substituted aryl or substituted heteroaryl. In embodiments, Ring A is a substituted aryl. In embodiments, Ring A is a substituted heteroaryl. In embodiments, Ring A is a substituted C₆-C₁₀ aryl. In embodiments, Ring A is a substituted C₁₀ aryl. In embodiments, Ring A is a substituted phenyl. In embodiments, Ring A is a substituted 5 to 10 membered heteroaryl. In embodiments, Ring A is a substituted 5 to 9 membered heteroaryl. In embodiments, Ring A is a substituted 5 to 6 membered heteroaryl. In embodiments, Ring A is a substituted 10 membered heteroaryl. In embodiments, Ring A is a substituted 9 membered heteroaryl. In embodiments, Ring A is a substituted 5 membered heteroaryl. In embodiments, Ring A is a substituted 6 membered heteroaryl. In embodiments, Ring A is a substituted phenyl. In embodiments, Ring A is a substituted pyridyl. In embodiments, Ring A is a substituted pyrazolyl. In embodiments, Ring A is a substituted imidazolyl. In embodiments, Ring A is a substituted oxazolyl. In embodiments, Ring A is a substituted isoxazolyl. In embodiments, Ring A is a substituted thiazolyl. In embodiments, Ring A is a substituted furanyl. In embodiments, Ring A is a substituted pyrrolyl. In embodiments, Ring A is a substituted thienyl. In embodiments, Ring A is a two fused ring aryl. In embodiments, Ring A is a two fused ring heteroaryl.

In embodiments, Ring A is a substituted phenyl or 5 to 6 membered heteroaryl. In embodiments, Ring A is a substituted phenyl. In embodiments, Ring A is a substituted 5 to 6 membered heteroaryl. In embodiments, Ring A is a substituted pyrazinyl, pyrimidinyl, furanyl, thienyl, pyrrolyl, imidazolyl, pyrazolyl, oxazolyl, isoxazolyl, or thiazolyl.

In embodiments, Ring A is a substituted phenyl. In embodiments, Ring A is a substituted pyridyl. In embodiments, Ring A is a substituted cyclohexyl. In embodiments, Ring A is a substituted morpholinyl. In embodiments, Ring A is a substituted piperazinyl. In embodiments, Ring A is a substituted furanyl. In embodiments, Ring A is a substituted thiazolyl. In embodiments, Ring A is a substituted pyrazolyl. In embodiments, Ring A is a substituted thienyl. In embodiments, Ring A is a substituted pyrazinyl. In embodiments, Ring A is a substituted pyrimidinyl. In embodiments, Ring A is a substituted pyridazinyl. In embodiments, Ring A is a substituted triazinyl. In embodiments, Ring A is a substituted tetrazinyl. In embodiments, Ring A is a substituted tetrazolyl. In embodiments, Ring A is a substituted triazolyl. In embodiments, Ring A is a substituted quinolinyl. In embodiments, Ring A is a substituted isoquinolinyl. In embodiments, Ring A is a substituted quinazolinyl. In embodiments, Ring A is a substituted quinoxalinyl. In embodiments, Ring A is a substituted imidazolyl. In embodiments, Ring A is a substituted oxazolyl. In embodiments, Ring A is a substituted isoxazolyl. In embodiments, Ring A is a substituted thiazolyl. In embodiments, Ring A is a substituted piperidinyl. In embodiments, Ring A is a substituted thiomorpholinyl. In embodiments, Ring A is a substituted thianyl. In embodiments, Ring A is a substituted oxanyl. In embodiments, Ring A is a substituted tetrahydropuranyl. In embodiments, Ring A is a substituted dihydropuranyl. In embodiments, Ring A is a substituted dioxanyl. In embodiments, Ring A is a substituted pyrazolyl. In embodiments, Ring A is a substituted pyrrolyl. In embodiments, Ring A is a substituted thienyl. In embodiments, Ring A is a substituted benzofuranyl. In embodiments, Ring A is a substituted indolyl. In embodiments, Ring A is a substituted benzothienyl. In embodiments, Ring A is a substituted benzimidazolyl. In embodiments, Ring A is a substituted isobenzofuranyl. In embodiments, Ring A is a substituted isoindolyl. In embodiments, Ring A is a substituted benzo[c]thienyl. In embodiments, Ring A is a substituted purinyl. In embodiments, Ring A is a substituted indazolyl. In embodiments, Ring A is a substituted benzoxazolyl. In embodiments, Ring A is a substituted benzisoxazolyl. In embodiments, Ring A is a substituted benzothiazolyl. In embodiments, Ring A is a substituted cyclopentyl. In embodiments, Ring A is a substituted cyclobutyl. In embodiments, Ring A is a substituted 2-thienyl. In embodiments, Ring A is a substituted 3-thienyl. In embodiments, Ring A is a substituted 2-furanyl. In embodiments, Ring A is a substituted 3-furanyl. In embodiments, Ring A is a substituted 2-pyridyl. In embodiments, Ring A is a substituted 3-pyridyl. In embodiments, Ring A is a substituted 4-pyridyl. In embodiments, Ring A is a substituted 3-pyrazolyl. In embodiments, Ring A is a substituted 4-pyrazolyl. In embodiments, Ring A is a substituted 5-pyrazolyl. In embodiments, Ring A is a substituted 2-pyrrolyl. In embodiments, Ring A is a substituted 3-pyrrolyl. In embodiments, Ring A is a substituted 2-thiazolyl. In embodiments, Ring A is a substituted 4-thiazolyl. In embodiments, Ring A is a substituted 5-thiazolyl. In embodiments, Ring A is a substituted 2-pyridyl. In embodiments, Ring A is a substituted 3-pyridyl. In embodiments, Ring A is a substituted 4-pyridyl. In embodiments, Ring A is a substituted phenyl.

In embodiments, Ring A is an unsubstituted aryl or heteroaryl. In embodiments, Ring A is an unsubstituted aryl. In embodiments, Ring A is an unsubstituted heteroaryl. In embodiments, Ring A is an unsubstituted C₆-C₁₀ aryl. In embodiments, Ring A is an unsubstituted C₁₀ aryl. In embodiments, Ring A is an unsubstituted phenyl. In embodiments, Ring A is an unsubstituted 5 to 10 membered heteroaryl. In embodiments, Ring A is an unsubstituted 5 to 9 membered heteroaryl. In embodiments, Ring A is an unsubstituted 5 to 6 membered heteroaryl. In embodiments, Ring A is an unsubstituted 10 membered heteroaryl. In embodiments, Ring A is an unsubstituted 9 membered heteroaryl. In embodiments, Ring A is an unsubstituted 5 membered heteroaryl. In embodiments, Ring A is an unsubstituted 6 membered heteroaryl. In embodiments, Ring A is an unsubstituted phenyl. In embodiments, Ring A is an unsubstituted pyridyl. In embodiments, Ring A is an unsubstituted pyrazolyl. In embodiments, Ring A is an unsubstituted imidazolyl. In embodiments, Ring A is an unsubstituted oxazolyl. In embodiments, Ring A is an unsubstituted isoxazolyl. In embodiments, Ring A is an unsubstituted thiazolyl. In embodiments, Ring A is an unsubstituted furanyl. In embodiments, Ring A is an unsubstituted pyrrolyl. In embodiments, Ring A is an unsubstituted thienyl. In embodiments, Ring A is a two fused ring aryl. In embodiments, Ring A is a two fused ring heteroaryl.

In embodiments, Ring A is an unsubstituted aryl or heteroaryl. In embodiments, Ring A is an unsubstituted phenyl or 5 to 6 membered heteroaryl. In embodiments, Ring A is an unsubstituted phenyl. In embodiments, Ring A is an unsubstituted 5 to 6 membered heteroaryl. In embodiments, Ring A is an unsubstituted pyrazinyl, pyrimidinyl, furanyl, thienyl, pyrrolyl, imidazolyl, pyrazolyl, oxazolyl, isoxazolyl, or thiazolyl.

In embodiments, Ring A is an unsubstituted phenyl. In embodiments, Ring A is an unsubstituted pyridyl. In embodiments, Ring A is an unsubstituted cyclohexyl. In embodiments, Ring A is an unsubstituted morpholinyl. In embodiments, Ring A is an unsubstituted piperazinyl. In embodiments, Ring A is an unsubstituted furanyl. In embodiments, Ring A is an unsubstituted thiazolyl. In embodiments, Ring A is an unsubstituted pyrazolyl. In embodiments, Ring A is an unsubstituted thienyl. In embodiments, Ring A is an unsubstituted pyrazinyl. In embodiments, Ring A is an unsubstituted pyrimidinyl. In embodiments, Ring A is an unsubstituted pyridazinyl. In embodiments, Ring A is an unsubstituted triazinyl. In embodiments, Ring A is an unsubstituted tetrazinyl. In embodiments, Ring A is an unsubstituted tetrazolyl. In embodiments, Ring A is an unsubstituted triazolyl. In embodiments, Ring A is an unsubstituted quinolinyl. In embodiments, Ring A is an unsubstituted isoquinolinyl. In embodiments, Ring A is an unsubstituted quinazolinyl. In embodiments, Ring A is an unsubstituted quinoxalinyl. In embodiments, Ring A is an unsubstituted imidazolyl. In embodiments, Ring A is an unsubstituted oxazolyl. In embodiments, Ring A is an unsubstituted isoxazolyl. In embodiments, Ring A is an unsubstituted thiazolyl. In embodiments, Ring A is an unsubstituted piperidinyl. In embodiments, Ring A is an unsubstituted thiomorpholinyl. In embodiments, Ring A is an unsubstituted thianyl. In embodiments, Ring A is an unsubstituted oxanyl. In embodiments, Ring A is an unsubstituted tetrahydropuranyl. In embodiments, Ring A is an unsubstituted dihydropuranyl. In embodiments, Ring A is an unsubstituted dioxanyl. In embodiments, Ring A is an unsubstituted pyrazolyl. In embodiments, Ring A is an unsubstituted pyrrolyl. In embodiments, Ring A is an unsubstituted thienyl. In embodiments, Ring A is an unsubstituted benzofuranyl. In embodiments, Ring A is an unsubstituted indolyl. In embodiments, Ring A is an unsubstituted benzothienyl. In embodiments, Ring A is an unsubstituted benzimidazolyl. In embodiments, Ring A is an unsubstituted isobenzofuranyl. In embodiments, Ring A is an unsubstituted isoindolyl. In embodiments, Ring A is an unsubstituted benzo[c]thienyl. In embodiments, Ring A is an unsubstituted purinyl. In embodiments, Ring A is an unsubstituted indazolyl. In embodiments, Ring A is an unsubstituted benzoxazolyl. In embodiments, Ring A is an unsubstituted benzisoxazolyl. In embodiments, Ring A is an unsubstituted benzothiazolyl. In embodiments, Ring A is an unsubstituted cyclopentyl. In embodiments, Ring A is an unsubstituted cyclobutyl. In embodiments, Ring A is an unsubstituted 2-thienyl. In embodiments, Ring A is an unsubstituted 3-thienyl. In embodiments, Ring A is an unsubstituted 2-furanyl. In embodiments, Ring A is an unsubstituted 3-furanyl. In embodiments, Ring A is an unsubstituted 2-pyridyl. In embodiments, Ring A is an unsubstituted 3-pyridyl. In embodiments, Ring A is an unsubstituted 4-pyridyl. In embodiments, Ring A is an unsubstituted 3-pyrazolyl. In embodiments, Ring A is an unsubstituted 4-pyrazolyl. In embodiments, Ring A is an unsubstituted 5-pyrazolyl. In embodiments, Ring A is an unsubstituted 2-pyrrolyl. In embodiments, Ring A is an unsubstituted 3-pyrrolyl. In embodiments, Ring A is an unsubstituted 2-thiazolyl. In embodiments, Ring A is an unsubstituted 4-thiazolyl. In embodiments, Ring A is an unsubstituted 5-thiazolyl. In embodiments, Ring A is an unsubstituted 2-pyridyl. In embodiments, Ring A is an unsubstituted 3-pyridyl. In embodiments, Ring A is an unsubstituted 4-pyridyl. In embodiments, Ring A is an unsubstituted phenyl.

In embodiments, Ring B is substituted or unsubstituted a cycloalkyl. In embodiments, Ring B is substituted or unsubstituted C₃-C₈ cycloalkyl. In embodiments, Ring B is substituted or unsubstituted C₃-C₇ cycloalkyl. In embodiments, Ring B is substituted or unsubstituted C₃-C₆ cycloalkyl. In embodiments, Ring B is substituted or unsubstituted C₃-C₅ cycloalkyl. In embodiments, Ring B is substituted or unsubstituted C₃-C₄ cycloalkyl. In embodiments, Ring B is substituted or unsubstituted C₄-C₈ cycloalkyl. In embodiments, Ring B is substituted or unsubstituted C₅-C₈ cycloalkyl. In embodiments, Ring B is substituted or unsubstituted C₆-C₈ cycloalkyl. In embodiments, Ring B is substituted or unsubstituted C₅-C₆ cycloalkyl. In embodiments, Ring B is substituted or unsubstituted C₃ cycloalkyl. In embodiments, Ring B is substituted or unsubstituted C₄ cycloalkyl. In embodiments, Ring B is substituted or unsubstituted C₅ cycloalkyl. In embodiments, Ring B is substituted or unsubstituted C₆ cycloalkyl. In embodiments, Ring B is substituted or unsubstituted C₇ cycloalkyl. In embodiments, Ring B is substituted or unsubstituted C₈ cycloalkyl. In embodiments, Ring B is substituted or unsubstituted cyclopropyl. In embodiments, Ring B is substituted or unsubstituted cyclobutyl. In embodiments, Ring B is substituted or unsubstituted cyclopentyl. In embodiments, Ring B is substituted or unsubstituted cyclohexyl. In embodiments, Ring B is substituted or unsubstituted cycloheptyl. In embodiments, Ring B is substituted or unsubstituted a heterocycloalkyl. In embodiments, Ring B is substituted or unsubstituted 3 to 8 membered heterocycloalkyl. In embodiments, Ring B is substituted or unsubstituted 3 to 7 membered heterocycloalkyl. In embodiments, Ring B is substituted or unsubstituted 3 to 6 membered heterocycloalkyl. In embodiments, Ring B is substituted or unsubstituted 3 to 5 membered heterocycloalkyl. In embodiments, Ring B is substituted or unsubstituted 3 to 4 membered heterocycloalkyl. In embodiments, Ring B is substituted or unsubstituted 4 to 8 membered heterocycloalkyl. In embodiments, Ring B is substituted or unsubstituted 5 to 8 membered heterocycloalkyl. In embodiments, Ring B is substituted or unsubstituted 6 to 8 membered heterocycloalkyl. In embodiments, Ring B is substituted or unsubstituted 5 to 6 membered heterocycloalkyl. In embodiments, Ring B is substituted or unsubstituted 3 membered heterocycloalkyl. In embodiments, Ring B is substituted or unsubstituted 4 membered heterocycloalkyl. In embodiments, Ring B is substituted or unsubstituted 5 membered heterocycloalkyl. In embodiments, Ring B is substituted or unsubstituted 6 membered heterocycloalkyl. In embodiments, Ring B is substituted or unsubstituted 7 membered heterocycloalkyl. In embodiments, Ring B is substituted or unsubstituted 8 membered heterocycloalkyl.

In embodiments, Ring B is substituted or unsubstituted aryl or heteroaryl. In embodiments, Ring B is substituted or unsubstituted aryl. In embodiments, Ring B is substituted or unsubstituted heteroaryl. In embodiments, Ring B is substituted or unsubstituted C₆-C₁₀ aryl. In embodiments, Ring B is substituted or unsubstituted C₁₀ aryl. In embodiments, Ring B is substituted or unsubstituted phenyl. In embodiments, Ring B is substituted or unsubstituted 5 to 10 membered heteroaryl. In embodiments, Ring B is substituted or unsubstituted 5 to 9 membered heteroaryl. In embodiments, Ring B is substituted or unsubstituted 5 to 6 membered heteroaryl. In embodiments, Ring B is substituted or unsubstituted 10 membered heteroaryl. In embodiments, Ring B is substituted or unsubstituted 9 membered heteroaryl. In embodiments, Ring B is substituted or unsubstituted 5 membered heteroaryl. In embodiments, Ring B is substituted or unsubstituted 6 membered heteroaryl. In embodiments, Ring B is substituted or unsubstituted phenyl. In embodiments, Ring B is substituted or unsubstituted pyridyl. In embodiments, Ring B is substituted or unsubstituted pyrazolyl. In embodiments, Ring B is substituted or unsubstituted imidazolyl. In embodiments, Ring B is substituted or unsubstituted oxazolyl. In embodiments, Ring B is substituted or unsubstituted isoxazolyl. In embodiments, Ring B is substituted or unsubstituted thiazolyl. In embodiments, Ring B is substituted or unsubstituted furanyl. In embodiments, Ring B is substituted or unsubstituted pyrrolyl. In embodiments, Ring B is substituted or unsubstituted thienyl. In embodiments, Ring B is a two fused ring aryl. In embodiments, Ring B is a two fused ring heteroaryl.

In embodiments, Ring B is substituted or unsubstituted aryl or heteroaryl. In embodiments, Ring B is substituted or unsubstituted phenyl or 5 to 6 membered heteroaryl. In embodiments, Ring B is substituted or unsubstituted phenyl. In embodiments, Ring B is substituted or unsubstituted 5 to 6 membered heteroaryl. In embodiments, Ring B is substituted or unsubstituted pyrazinyl, pyrimidinyl, furanyl, thienyl, pyrrolyl, imidazolyl, pyrazolyl, oxazolyl, isoxazolyl, or thiazolyl.

In embodiments, Ring B is substituted or unsubstituted phenyl. In embodiments, Ring B is substituted or unsubstituted pyridyl. In embodiments, Ring B is substituted or unsubstituted cyclohexyl. In embodiments, Ring B is substituted or unsubstituted morpholinyl. In embodiments, Ring B is substituted or unsubstituted piperazinyl. In embodiments, Ring B is substituted or unsubstituted furanyl. In embodiments, Ring B is substituted or unsubstituted thiazolyl. In embodiments, Ring B is substituted or unsubstituted pyrazolyl. In embodiments, Ring B is substituted or unsubstituted thienyl. In embodiments, Ring B is substituted or unsubstituted pyrazinyl. In embodiments, Ring B is substituted or unsubstituted pyrimidinyl. In embodiments, Ring B is substituted or unsubstituted pyridazinyl. In embodiments, Ring B is substituted or unsubstituted triazinyl. In embodiments, Ring B is substituted or unsubstituted tetrazinyl. In embodiments, Ring B is substituted or unsubstituted tetrazolyl. In embodiments, Ring B is substituted or unsubstituted triazolyl. In embodiments, Ring B is substituted or unsubstituted quinolinyl. In embodiments, Ring B is substituted or unsubstituted isoquinolinyl. In embodiments, Ring B is substituted or unsubstituted quinazolinyl. In embodiments, Ring B is substituted or unsubstituted quinoxalinyl. In embodiments, Ring B is substituted or unsubstituted imidazolyl. In embodiments, Ring B is substituted or unsubstituted oxazolyl. In embodiments, Ring B is substituted or unsubstituted isoxazolyl. In embodiments, Ring B is substituted or unsubstituted thiazolyl. In embodiments, Ring B is substituted or unsubstituted piperidinyl. In embodiments, Ring B is substituted or unsubstituted thiomorpholinyl. In embodiments, Ring B is substituted or unsubstituted thianyl. In embodiments, Ring B is substituted or unsubstituted oxanyl. In embodiments, Ring B is substituted or unsubstituted tetrahydropuranyl. In embodiments, Ring B is substituted or unsubstituted dihydropuranyl. In embodiments, Ring B is substituted or unsubstituted dioxanyl. In embodiments, Ring B is substituted or unsubstituted pyrazolyl. In embodiments, Ring B is substituted or unsubstituted pyrrolyl. In embodiments, Ring B is substituted or unsubstituted thienyl. In embodiments, Ring B is substituted or unsubstituted benzofuranyl. In embodiments, Ring B is substituted or unsubstituted indolyl. In embodiments, Ring B is substituted or unsubstituted benzothienyl. In embodiments, Ring B is substituted or unsubstituted benzimidazolyl. In embodiments, Ring B is substituted or unsubstituted isobenzofuranyl. In embodiments, Ring B is substituted or unsubstituted isoindolyl. In embodiments, Ring B is substituted or unsubstituted benzo[c]thienyl. In embodiments, Ring B is substituted or unsubstituted purinyl. In embodiments, Ring B is substituted or unsubstituted indazolyl. In embodiments, Ring B is substituted or unsubstituted benzoxazolyl. In embodiments, Ring B is substituted or unsubstituted benzisoxazolyl. In embodiments, Ring B is substituted or unsubstituted benzothiazolyl. In embodiments, Ring B is substituted or unsubstituted cyclopentyl. In embodiments, Ring B is substituted or unsubstituted cyclobutyl. In embodiments, Ring B is substituted or unsubstituted 2-thienyl. In embodiments, Ring B is substituted or unsubstituted 3-thienyl. In embodiments, Ring B is substituted or unsubstituted 2-furanyl. In embodiments, Ring B is substituted or unsubstituted 3-furanyl. In embodiments, Ring B is substituted or unsubstituted 2-pyridyl. In embodiments, Ring B is substituted or unsubstituted 3-pyridyl. In embodiments, Ring B is substituted or unsubstituted 4-pyridyl. In embodiments, Ring B is substituted or unsubstituted 3-pyrazolyl. In embodiments, Ring B is substituted or unsubstituted 4-pyrazolyl. In embodiments, Ring B is substituted or unsubstituted 5-pyrazolyl. In embodiments, Ring B is substituted or unsubstituted 2-pyrrolyl. In embodiments, Ring B is substituted or unsubstituted 3-pyrrolyl. In embodiments, Ring B is substituted or unsubstituted 2-thiazolyl. In embodiments, Ring B is substituted or unsubstituted 4-thiazolyl. In embodiments, Ring B is substituted or unsubstituted 5-thiazolyl. In embodiments, Ring B is substituted or unsubstituted 2-pyridyl. In embodiments, Ring B is substituted or unsubstituted 3-pyridyl. In embodiments, Ring B is substituted or unsubstituted 4-pyridyl. In embodiments, Ring B is substituted or unsubstituted phenyl.

In embodiments, Ring B is an unsubstituted cycloalkyl. In embodiments, Ring B is an unsubstituted C₃-C₈ cycloalkyl. In embodiments, Ring B is an unsubstituted C₃-C₇ cycloalkyl. In embodiments, Ring B is an unsubstituted C₃-C₆ cycloalkyl. In embodiments, Ring B is an unsubstituted C₃-C₅ cycloalkyl. In embodiments, Ring B is an unsubstituted C₃-C₄ cycloalkyl. In embodiments, Ring B is an unsubstituted C₄-C₈ cycloalkyl. In embodiments, Ring B is an unsubstituted C₅-C₈ cycloalkyl. In embodiments, Ring B is an unsubstituted C₆-C₈ cycloalkyl. In embodiments, Ring B is an unsubstituted C₅-C₆ cycloalkyl. In embodiments, Ring B is an unsubstituted C₃ cycloalkyl. In embodiments, Ring B is an unsubstituted C₄ cycloalkyl. In embodiments, Ring B is an unsubstituted C₅ cycloalkyl. In embodiments, Ring B is an unsubstituted C₆ cycloalkyl. In embodiments, Ring B is an unsubstituted C₇ cycloalkyl. In embodiments, Ring B is an unsubstituted C₈ cycloalkyl. In embodiments, Ring B is an unsubstituted cyclopropyl. In embodiments, Ring B is an unsubstituted cyclobutyl. In embodiments, Ring B is an unsubstituted cyclopentyl. In embodiments, Ring B is an unsubstituted cyclohexyl. In embodiments, Ring B is an unsubstituted cycloheptyl. In embodiments, Ring B is an unsubstituted a heterocycloalkyl. In embodiments, Ring B is an unsubstituted 3 to 8 membered heterocycloalkyl. In embodiments, Ring B is an unsubstituted 3 to 7 membered heterocycloalkyl. In embodiments, Ring B is an unsubstituted 3 to 6 membered heterocycloalkyl. In embodiments, Ring B is an unsubstituted 3 to 5 membered heterocycloalkyl. In embodiments, Ring B is an unsubstituted 3 to 4 membered heterocycloalkyl. In embodiments, Ring B is an unsubstituted 4 to 8 membered heterocycloalkyl. In embodiments, Ring B is an unsubstituted 5 to 8 membered heterocycloalkyl. In embodiments, Ring B is an unsubstituted 6 to 8 membered heterocycloalkyl. In embodiments, Ring B is an unsubstituted 5 to 6 membered heterocycloalkyl. In embodiments, Ring B is an unsubstituted 3 membered heterocycloalkyl. In embodiments, Ring B is an unsubstituted 4 membered heterocycloalkyl. In embodiments, Ring B is an unsubstituted 5 membered heterocycloalkyl. In embodiments, Ring B is an unsubstituted 6 membered heterocycloalkyl. In embodiments, Ring B is an unsubstituted 7 membered heterocycloalkyl. In embodiments, Ring B is an unsubstituted 8 membered heterocycloalkyl. In embodiments, Ring B is an unsubstituted aryl or unsubstituted heteroaryl. In embodiments, Ring B is an unsubstituted aryl. In embodiments, Ring B is an unsubstituted heteroaryl. In embodiments, Ring B is an unsubstituted C₆-C₁₀ aryl. In embodiments, Ring B is an unsubstituted C₁₀ aryl. In embodiments, Ring B is an unsubstituted phenyl. In embodiments, Ring B is an unsubstituted 5 to 10 membered heteroaryl. In embodiments, Ring B is an unsubstituted 5 to 9 membered heteroaryl. In embodiments, Ring B is an unsubstituted 5 to 6 membered heteroaryl. In embodiments, Ring B is an unsubstituted 10 membered heteroaryl. In embodiments, Ring B is an unsubstituted 9 membered heteroaryl. In embodiments, Ring B is an unsubstituted 5 membered heteroaryl. In embodiments, Ring B is an unsubstituted 6 membered heteroaryl. In embodiments, Ring B is an unsubstituted phenyl. In embodiments, Ring B is an unsubstituted pyridyl. In embodiments, Ring B is an unsubstituted pyrazolyl. In embodiments, Ring B is an unsubstituted imidazolyl. In embodiments, Ring B is an unsubstituted oxazolyl. In embodiments, Ring B is an unsubstituted isoxazolyl. In embodiments, Ring B is an unsubstituted thiazolyl. In embodiments, Ring B is an unsubstituted furanyl. In embodiments, Ring B is an unsubstituted pyrrolyl. In embodiments, Ring B is an unsubstituted thienyl. In embodiments, Ring B is an unsubstituted two fused ring aryl. In embodiments, Ring B is an unsubstituted two fused ring heteroaryl.

In embodiments, Ring B is an unsubstituted aryl or unsubstituted heteroaryl. In embodiments, Ring B is an unsubstituted phenyl or unsubstituted 5 to 6 membered heteroaryl. In embodiments, Ring B is an unsubstituted phenyl. In embodiments, Ring B is an unsubstituted 5 to 6 membered heteroaryl. In embodiments, Ring B is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

In embodiments, Ring B is an unsubstituted phenyl. In embodiments, Ring B is an unsubstituted pyridyl. In embodiments, Ring B is an unsubstituted cyclohexyl. In embodiments, Ring B is an unsubstituted morpholinyl. In embodiments, Ring B is an unsubstituted piperazinyl. In embodiments, Ring B is an unsubstituted furanyl. In embodiments, Ring B is an unsubstituted thiazolyl. In embodiments, Ring B is an unsubstituted pyrazolyl. In embodiments, Ring B is an unsubstituted thienyl. In embodiments, Ring B is an unsubstituted pyrazinyl. In embodiments, Ring B is an unsubstituted pyrimidinyl. In embodiments, Ring B is an unsubstituted pyridazinyl. In embodiments, Ring B is an unsubstituted triazinyl. In embodiments, Ring B is an unsubstituted tetrazinyl. In embodiments, Ring B is an unsubstituted tetrazolyl. In embodiments, Ring B is an unsubstituted triazolyl. In embodiments, Ring B is an unsubstituted quinolinyl. In embodiments, Ring B is an unsubstituted isoquinolinyl. In embodiments, Ring B is an unsubstituted quinazolinyl. In embodiments, Ring B is an unsubstituted quinoxalinyl. In embodiments, Ring B is an unsubstituted imidazolyl. In embodiments, Ring B is an unsubstituted oxazolyl. In embodiments, Ring B is an unsubstituted isoxazolyl. In embodiments, Ring B is an unsubstituted thiazolyl. In embodiments, Ring B is an unsubstituted piperidinyl. In embodiments, Ring B is an unsubstituted thiomorpholinyl. In embodiments, Ring B is an unsubstituted thianyl. In embodiments, Ring B is an unsubstituted oxanyl. In embodiments, Ring B is an unsubstituted tetrahydropuranyl. In embodiments, Ring B is an unsubstituted dihydropuranyl. In embodiments, Ring B is an unsubstituted dioxanyl. In embodiments, Ring B is an unsubstituted pyrazolyl. In embodiments, Ring B is an unsubstituted pyrrolyl. In embodiments, Ring B is an unsubstituted thienyl. In embodiments, Ring B is an unsubstituted benzofuranyl. In embodiments, Ring B is an unsubstituted indolyl. In embodiments, Ring B is an unsubstituted benzothienyl. In embodiments, Ring B is an unsubstituted benzimidazolyl. In embodiments, Ring B is an unsubstituted isobenzofuranyl. In embodiments, Ring B is an unsubstituted isoindolyl. In embodiments, Ring B is an unsubstituted benzo[c]thienyl. In embodiments, Ring B is an unsubstituted purinyl. In embodiments, Ring B is an unsubstituted indazolyl. In embodiments, Ring B is an unsubstituted benzoxazolyl. In embodiments, Ring B is an unsubstituted benzisoxazolyl. In embodiments, Ring B is an unsubstituted benzothiazolyl. In embodiments, Ring B is an unsubstituted cyclopentyl. In embodiments, Ring B is an unsubstituted cyclobutyl. In embodiments, Ring B is an unsubstituted 2-thienyl. In embodiments, Ring B is an unsubstituted 3-thienyl. In embodiments, Ring B is an unsubstituted 2-furanyl. In embodiments, Ring B is an unsubstituted 3-furanyl. In embodiments, Ring B is an unsubstituted 2-pyridyl. In embodiments, Ring B is an unsubstituted 3-pyridyl. In embodiments, Ring B is an unsubstituted 4-pyridyl. In embodiments, Ring B is an unsubstituted 3-pyrazolyl. In embodiments, Ring B is an unsubstituted 4-pyrazolyl. In embodiments, Ring B is an unsubstituted 5-pyrazolyl. In embodiments, Ring B is an unsubstituted 2-pyrrolyl. In embodiments, Ring B is an unsubstituted 3-pyrrolyl. In embodiments, Ring B is an unsubstituted 2-thiazolyl. In embodiments, Ring B is an unsubstituted 4-thiazolyl. In embodiments, Ring B is an unsubstituted 5-thiazolyl. It will be understood that an unsubstituted Ring B does not have substituents in addition to the bond to L³ and bonds to any R⁴ substituents.

In embodiments, Ring C is substituted or unsubstituted a cycloalkyl. In embodiments, Ring C is substituted or unsubstituted C₃-C₈ cycloalkyl. In embodiments, Ring C is substituted or unsubstituted C₃-C₇ cycloalkyl. In embodiments, Ring C is substituted or unsubstituted C₃-C₆ cycloalkyl. In embodiments, Ring C is substituted or unsubstituted C₃-C₅ cycloalkyl. In embodiments, Ring C is substituted or unsubstituted C₃-C₄ cycloalkyl. In embodiments, Ring C is substituted or unsubstituted C₄-C₈ cycloalkyl. In embodiments, Ring C is substituted or unsubstituted C₅-C₈ cycloalkyl. In embodiments, Ring C is substituted or unsubstituted C₆-C₈ cycloalkyl. In embodiments, Ring C is substituted or unsubstituted C₅-C₆ cycloalkyl. In embodiments, Ring C is substituted or unsubstituted C₃ cycloalkyl. In embodiments, Ring C is substituted or unsubstituted C₄ cycloalkyl. In embodiments, Ring C is substituted or unsubstituted C₅ cycloalkyl. In embodiments, Ring C is substituted or unsubstituted C₆ cycloalkyl. In embodiments, Ring C is substituted or unsubstituted C₇ cycloalkyl. In embodiments, Ring C is substituted or unsubstituted C₈ cycloalkyl. In embodiments, Ring C is substituted or unsubstituted cyclopropyl. In embodiments, Ring C is substituted or unsubstituted cyclobutyl. In embodiments, Ring C is substituted or unsubstituted cyclopentyl. In embodiments, Ring C is substituted or unsubstituted cyclohexyl. In embodiments, Ring C is substituted or unsubstituted cycloheptyl. In embodiments, Ring C is substituted or unsubstituted a heterocycloalkyl. In embodiments, Ring C is substituted or unsubstituted 3 to 8 membered heterocycloalkyl. In embodiments, Ring C is substituted or unsubstituted 3 to 7 membered heterocycloalkyl. In embodiments, Ring C is substituted or unsubstituted 3 to 6 membered heterocycloalkyl. In embodiments, Ring C is substituted or unsubstituted 3 to 5 membered heterocycloalkyl. In embodiments, Ring C is substituted or unsubstituted 3 to 4 membered heterocycloalkyl. In embodiments, Ring C is substituted or unsubstituted 4 to 8 membered heterocycloalkyl. In embodiments, Ring C is substituted or unsubstituted 5 to 8 membered heterocycloalkyl. In embodiments, Ring C is substituted or unsubstituted 6 to 8 membered heterocycloalkyl. In embodiments, Ring C is substituted or unsubstituted 5 to 6 membered heterocycloalkyl. In embodiments, Ring C is substituted or unsubstituted 3 membered heterocycloalkyl. In embodiments, Ring C is substituted or unsubstituted 4 membered heterocycloalkyl. In embodiments, Ring C is substituted or unsubstituted 5 membered heterocycloalkyl. In embodiments, Ring C is substituted or unsubstituted 6 membered heterocycloalkyl. In embodiments, Ring C is substituted or unsubstituted 7 membered heterocycloalkyl. In embodiments, Ring C is substituted or unsubstituted 8 membered heterocycloalkyl. In embodiments, Ring C is substituted or unsubstituted aryl or heteroaryl. In embodiments, Ring C is substituted or unsubstituted aryl. In embodiments, Ring C is substituted or unsubstituted heteroaryl. In embodiments, Ring C is substituted or unsubstituted C₆-C₁₀ aryl. In embodiments, Ring C is substituted or unsubstituted C₁₀ aryl. In embodiments, Ring C is substituted or unsubstituted phenyl. In embodiments, Ring C is substituted or unsubstituted 5 to 10 membered heteroaryl. In embodiments, Ring C is substituted or unsubstituted 5 to 9 membered heteroaryl. In embodiments, Ring C is substituted or unsubstituted 5 to 6 membered heteroaryl. In embodiments, Ring C is substituted or unsubstituted 10 membered heteroaryl. In embodiments, Ring C is substituted or unsubstituted 9 membered heteroaryl. In embodiments, Ring C is substituted or unsubstituted 5 membered heteroaryl. In embodiments, Ring C is substituted or unsubstituted 6 membered heteroaryl. In embodiments, Ring C is substituted or unsubstituted phenyl. In embodiments, Ring C is substituted or unsubstituted pyridyl. In embodiments, Ring C is substituted or unsubstituted pyrazolyl. In embodiments, Ring C is substituted or unsubstituted imidazolyl. In embodiments, Ring C is substituted or unsubstituted oxazolyl. In embodiments, Ring C is substituted or unsubstituted is substituted or unsubstitutedoxazolyl. In embodiments, Ring C is substituted or unsubstituted thiazolyl. In embodiments, Ring C is substituted or unsubstituted furanyl. In embodiments, Ring C is substituted or unsubstituted pyrrolyl. In embodiments, Ring C is substituted or unsubstituted thienyl. In embodiments, Ring C is a two fused ring aryl. In embodiments, Ring C is a two fused ring heteroaryl.

In embodiments, Ring C is substituted or unsubstituted aryl or heteroaryl. In embodiments, Ring C is substituted or unsubstituted phenyl or 5 to 6 membered heteroaryl. In embodiments, Ring C is substituted or unsubstituted phenyl. In embodiments, Ring C is substituted or unsubstituted 5 to 6 membered heteroaryl. In embodiments, Ring C is substituted or unsubstituted pyrazinyl, pyrimidinyl, furanyl, thienyl, pyrrolyl, imidazolyl, pyrazolyl, oxazolyl, isoxazolyl, or thiazolyl.

In embodiments, Ring C is substituted or unsubstituted phenyl. In embodiments, Ring C is substituted or unsubstituted pyridyl. In embodiments, Ring C is substituted or unsubstituted cyclohexyl. In embodiments, Ring C is substituted or unsubstituted morpholinyl. In embodiments, Ring C is substituted or unsubstituted piperazinyl. In embodiments, Ring C is substituted or unsubstituted furanyl. In embodiments, Ring C is substituted or unsubstituted thiazolyl. In embodiments, Ring C is substituted or unsubstituted pyrazolyl. In embodiments, Ring C is substituted or unsubstituted thienyl. In embodiments, Ring C is substituted or unsubstituted pyrazinyl. In embodiments, Ring C is substituted or unsubstituted pyrimidinyl. In embodiments, Ring C is substituted or unsubstituted pyridazinyl. In embodiments, Ring C is substituted or unsubstituted triazinyl. In embodiments, Ring C is substituted or unsubstituted tetrazinyl. In embodiments, Ring C is substituted or unsubstituted tetrazolyl. In embodiments, Ring C is substituted or unsubstituted triazolyl. In embodiments, Ring C is substituted or unsubstituted quinolinyl. In embodiments, Ring C is substituted or unsubstituted isoquinolinyl. In embodiments, Ring C is substituted or unsubstituted quinazolinyl. In embodiments, Ring C is substituted or unsubstituted quinoxalinyl. In embodiments, Ring C is substituted or unsubstituted imidazolyl. In embodiments, Ring C is substituted or unsubstituted oxazolyl. In embodiments, Ring C is substituted or unsubstituted isoxazolyl. In embodiments, Ring C is substituted or unsubstituted thiazolyl. In embodiments, Ring C is substituted or unsubstituted piperidinyl. In embodiments, Ring C is substituted or unsubstituted thiomorpholinyl. In embodiments, Ring C is substituted or unsubstituted thianyl. In embodiments, Ring C is substituted or unsubstituted oxanyl. In embodiments, Ring C is substituted or unsubstituted tetrahydropuranyl. In embodiments, Ring C is substituted or unsubstituted dihydropuranyl. In embodiments, Ring C is substituted or unsubstituted dioxanyl. In embodiments, Ring C is substituted or unsubstituted pyrazolyl. In embodiments, Ring C is substituted or unsubstituted pyrrolyl. In embodiments, Ring C is substituted or unsubstituted thienyl. In embodiments, Ring C is substituted or unsubstituted benzofuranyl. In embodiments, Ring C is substituted or unsubstituted indolyl. In embodiments, Ring C is substituted or unsubstituted benzothienyl. In embodiments, Ring C is substituted or unsubstituted benzimidazolyl. In embodiments, Ring C is substituted or unsubstituted isobenzofuranyl. In embodiments, Ring C is substituted or unsubstituted isoindolyl. In embodiments, Ring C is substituted or unsubstituted benzo[c]thienyl. In embodiments, Ring C is substituted or unsubstituted purinyl. In embodiments, Ring C is substituted or unsubstituted indazolyl. In embodiments, Ring C is substituted or unsubstituted benzoxazolyl. In embodiments, Ring C is substituted or unsubstituted benzisoxazolyl. In embodiments, Ring C is substituted or unsubstituted benzothiazolyl. In embodiments, Ring C is substituted or unsubstituted cyclopentyl. In embodiments, Ring C is substituted or unsubstituted cyclobutyl. In embodiments, Ring C is substituted or unsubstituted 2-thienyl. In embodiments, Ring C is substituted or unsubstituted 3-thienyl. In embodiments, Ring C is substituted or unsubstituted 2-furanyl. In embodiments, Ring C is substituted or unsubstituted 3-furanyl. In embodiments, Ring C is substituted or unsubstituted 2-pyridyl. In embodiments, Ring C is substituted or unsubstituted 3-pyridyl. In embodiments, Ring C is substituted or unsubstituted 4-pyridyl. In embodiments, Ring C is substituted or unsubstituted 3-pyrazolyl. In embodiments, Ring C is substituted or unsubstituted 4-pyrazolyl. In embodiments, Ring C is substituted or unsubstituted 5-pyrazolyl. In embodiments, Ring C is substituted or unsubstituted 2-pyrrolyl. In embodiments, Ring C is substituted or unsubstituted 3-pyrrolyl. In embodiments, Ring C is substituted or unsubstituted 2-thiazolyl. In embodiments, Ring C is substituted or unsubstituted 4-thiazolyl. In embodiments, Ring C is substituted or unsubstituted 5-thiazolyl. In embodiments, Ring C is substituted or unsubstituted 2-pyridyl. In embodiments, Ring C is substituted or unsubstituted 3-pyridyl. In embodiments, Ring C is substituted or unsubstituted 4-pyridyl. In embodiments, Ring C is substituted or unsubstituted phenyl.

In embodiments, Ring C is an unsubstituted cycloalkyl. In embodiments, Ring C is an unsubstituted C₃-C₈ cycloalkyl. In embodiments, Ring C is an unsubstituted C₃-C₇ cycloalkyl. In embodiments, Ring C is an unsubstituted C₃-C₆ cycloalkyl. In embodiments, Ring C is an unsubstituted C₃-C₅ cycloalkyl. In embodiments, Ring C is an unsubstituted C₃-C₄ cycloalkyl. In embodiments, Ring C is an unsubstituted C₄-C₈ cycloalkyl. In embodiments, Ring C is an unsubstituted C₅-C₈ cycloalkyl. In embodiments, Ring C is an unsubstituted C₆-C₈ cycloalkyl. In embodiments, Ring C is an unsubstituted C₅-C₆ cycloalkyl. In embodiments, Ring C is an unsubstituted C₃ cycloalkyl. In embodiments, Ring C is an unsubstituted C₄ cycloalkyl. In embodiments, Ring C is an unsubstituted C₅ cycloalkyl. In embodiments, Ring C is an unsubstituted C₆ cycloalkyl. In embodiments, Ring C is an unsubstituted C₇ cycloalkyl. In embodiments, Ring C is an unsubstituted C₈ cycloalkyl. In embodiments, Ring C is an unsubstituted cyclopropyl. In embodiments, Ring C is an unsubstituted cyclobutyl. In embodiments, Ring C is an unsubstituted cyclopentyl. In embodiments, Ring C is an unsubstituted cyclohexyl. In embodiments, Ring C is an unsubstituted cycloheptyl. In embodiments, Ring C is an unsubstituted a heterocycloalkyl. In embodiments, Ring C is an unsubstituted 3 to 8 membered heterocycloalkyl. In embodiments, Ring C is an unsubstituted 3 to 7 membered heterocycloalkyl. In embodiments, Ring C is an unsubstituted 3 to 6 membered heterocycloalkyl. In embodiments, Ring C is an unsubstituted 3 to 5 membered heterocycloalkyl. In embodiments, Ring C is an unsubstituted 3 to 4 membered heterocycloalkyl. In embodiments, Ring C is an unsubstituted 4 to 8 membered heterocycloalkyl. In embodiments, Ring C is an unsubstituted 5 to 8 membered heterocycloalkyl. In embodiments, Ring C is an unsubstituted 6 to 8 membered heterocycloalkyl. In embodiments, Ring C is an unsubstituted 5 to 6 membered heterocycloalkyl. In embodiments, Ring C is an unsubstituted 3 membered heterocycloalkyl. In embodiments, Ring C is an unsubstituted 4 membered heterocycloalkyl. In embodiments, Ring C is an unsubstituted 5 membered heterocycloalkyl. In embodiments, Ring C is an unsubstituted 6 membered heterocycloalkyl. In embodiments, Ring C is an unsubstituted 7 membered heterocycloalkyl. In embodiments, Ring C is an unsubstituted 8 membered heterocycloalkyl. In embodiments, Ring C is an unsubstituted aryl or unsubstituted heteroaryl. In embodiments, Ring C is an unsubstituted aryl. In embodiments, Ring C is an unsubstituted heteroaryl. In embodiments, Ring C is an unsubstituted C₆-C₁₀ aryl. In embodiments, Ring C is an unsubstituted C₁₀ aryl. In embodiments, Ring C is an unsubstituted phenyl. In embodiments, Ring C is an unsubstituted 5 to 10 membered heteroaryl. In embodiments, Ring C is an unsubstituted 5 to 9 membered heteroaryl. In embodiments, Ring C is an unsubstituted 5 to 6 membered heteroaryl. In embodiments, Ring C is an unsubstituted 10 membered heteroaryl. In embodiments, Ring C is an unsubstituted 9 membered heteroaryl. In embodiments, Ring C is an unsubstituted 5 membered heteroaryl. In embodiments, Ring C is an unsubstituted 6 membered heteroaryl. In embodiments, Ring C is an unsubstituted phenyl. In embodiments, Ring C is an unsubstituted pyridyl. In embodiments, Ring C is an unsubstituted pyrazolyl. In embodiments, Ring C is an unsubstituted imidazolyl. In embodiments, Ring C is an unsubstituted oxazolyl. In embodiments, Ring C is an unsubstituted isoxazolyl. In embodiments, Ring C is an unsubstituted thiazolyl. In embodiments, Ring C is an unsubstituted furanyl. In embodiments, Ring C is an unsubstituted pyrrolyl. In embodiments, Ring C is an unsubstituted thienyl. In embodiments, Ring C is an unsubstituted two fused ring aryl. In embodiments, Ring C is an unsubstituted two fused ring heteroaryl.

In embodiments, Ring C is an unsubstituted aryl or unsubstituted heteroaryl. In embodiments, Ring C is an unsubstituted phenyl or unsubstituted 5 to 6 membered heteroaryl. In embodiments, Ring C is an unsubstituted phenyl. In embodiments, Ring C is an unsubstituted 5 to 6 membered heteroaryl. In embodiments, Ring C is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

In embodiments, Ring C is an unsubstituted phenyl. In embodiments, Ring C is an unsubstituted pyridyl. In embodiments, Ring C is an unsubstituted cyclohexyl. In embodiments, Ring C is an unsubstituted morpholinyl. In embodiments, Ring C is an unsubstituted piperazinyl. In embodiments, Ring C is an unsubstituted furanyl. In embodiments, Ring C is an unsubstituted thiazolyl. In embodiments, Ring C is an unsubstituted pyrazolyl. In embodiments, Ring C is an unsubstituted thienyl. In embodiments, Ring C is an unsubstituted pyrazinyl. In embodiments, Ring C is an unsubstituted pyrimidinyl. In embodiments, Ring C is an unsubstituted pyridazinyl. In embodiments, Ring C is an unsubstituted triazinyl. In embodiments, Ring C is an unsubstituted tetrazinyl. In embodiments, Ring C is an unsubstituted tetrazolyl. In embodiments, Ring C is an unsubstituted triazolyl. In embodiments, Ring C is an unsubstituted quinolinyl. In embodiments, Ring C is an unsubstituted isoquinolinyl. In embodiments, Ring C is an unsubstituted quinazolinyl. In embodiments, Ring C is an unsubstituted quinoxalinyl. In embodiments, Ring C is an unsubstituted imidazolyl. In embodiments, Ring C is an unsubstituted oxazolyl. In embodiments, Ring C is an unsubstituted isoxazolyl. In embodiments, Ring C is an unsubstituted thiazolyl. In embodiments, Ring C is an unsubstituted piperidinyl. In embodiments, Ring C is an unsubstituted thiomorpholinyl. In embodiments, Ring C is an unsubstituted thianyl. In embodiments, Ring C is an unsubstituted oxanyl. In embodiments, Ring C is an unsubstituted tetrahydropuranyl. In embodiments, Ring C is an unsubstituted dihydropuranyl. In embodiments, Ring C is an unsubstituted dioxanyl. In embodiments, Ring C is an unsubstituted pyrazolyl. In embodiments, Ring C is an unsubstituted pyrrolyl. In embodiments, Ring C is an unsubstituted thienyl. In embodiments, Ring C is an unsubstituted benzofuranyl. In embodiments, Ring C is an unsubstituted indolyl. In embodiments, Ring C is an unsubstituted benzothienyl. In embodiments, Ring C is an unsubstituted benzimidazolyl. In embodiments, Ring C is an unsubstituted isobenzofuranyl. In embodiments, Ring C is an unsubstituted isoindolyl. In embodiments, Ring C is an unsubstituted benzo[c]thienyl. In embodiments, Ring C is an unsubstituted purinyl. In embodiments, Ring C is an unsubstituted indazolyl. In embodiments, Ring C is an unsubstituted benzoxazolyl. In embodiments, Ring C is an unsubstituted benzisoxazolyl. In embodiments, Ring C is an unsubstituted benzothiazolyl. In embodiments, Ring C is an unsubstituted cyclopentyl. In embodiments, Ring C is an unsubstituted cyclobutyl. In embodiments, Ring C is an unsubstituted 2-thienyl. In embodiments, Ring C is an unsubstituted 3-thienyl. In embodiments, Ring C is an unsubstituted 2-furanyl. In embodiments, Ring C is an unsubstituted 3-furanyl. In embodiments, Ring C is an unsubstituted 2-pyridyl. In embodiments, Ring C is an unsubstituted 3-pyridyl. In embodiments, Ring C is an unsubstituted 4-pyridyl. In embodiments, Ring C is an unsubstituted 3-pyrazolyl. In embodiments, Ring C is an unsubstituted 4-pyrazolyl. In embodiments, Ring C is an unsubstituted 5-pyrazolyl. In embodiments, Ring C is an unsubstituted 2-pyrrolyl. In embodiments, Ring C is an unsubstituted 3-pyrrolyl. In embodiments, Ring C is an unsubstituted 2-thiazolyl. In embodiments, Ring C is an unsubstituted 4-thiazolyl. In embodiments, Ring C is an unsubstituted 5-thiazolyl.

In embodiments, R¹ is -L¹-L²-E, substituted or unsubstituted aryl, or substituted or unsubstituted heteroaryl. In embodiments, R¹ is hydrogen. In embodiments, R¹ is substituted or unsubstituted aryl or substituted or unsubstituted heteroaryl. In embodiments, R¹ is substituted or unsubstituted phenyl or substituted or unsubstituted 5 to 6 membered heteroaryl. In embodiments, R¹ is substituted or unsubstituted phenyl. In embodiments, R¹ is an unsubstituted phenyl. In embodiments, R¹ is substituted or unsubstituted 5 to 6 membered heteroaryl. In embodiments, R¹ is an unsubstituted 5 to 6 membered heteroaryl. In embodiments, R¹ is substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted oxazolyl, substituted or unsubstituted isoxazolyl, or substituted or unsubstituted thiazolyl. In embodiments, R¹ is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl. In embodiments, R¹ is -L¹-L²-E.

In embodiments, R¹ is substituted phenyl. In embodiments, R¹ is substituted pyridyl. In embodiments, R¹ is substituted cyclohexyl. In embodiments, R¹ is substituted morpholinyl. In embodiments, R¹ is substituted piperazinyl. In embodiments, R¹ is substituted furanyl. In embodiments, R¹ is substituted thiazolyl. In embodiments, R¹ is substituted pyrazolyl. In embodiments, R¹ is substituted thienyl. In embodiments, R¹ is substituted pyrazinyl. In embodiments, R¹ is substituted pyrimidinyl. In embodiments, R¹ is substituted pyridazinyl. In embodiments, R¹ is substituted triazinyl. In embodiments, R¹ is substituted tetrazinyl. In embodiments, R¹ is substituted tetrazolyl. In embodiments, R¹ is substituted triazolyl. In embodiments, R¹ is substituted quinolinyl. In embodiments, R¹ is substituted isoquinolinyl. In embodiments, R¹ is substituted quinazolinyl. In embodiments, R¹ is substituted quinoxalinyl. In embodiments, R¹ is substituted imidazolyl. In embodiments, R¹ is substituted oxazolyl. In embodiments, R¹ is substituted isoxazolyl. In embodiments, R¹ is substituted thiazolyl. In embodiments, R¹ is substituted piperidinyl. In embodiments, R¹ is substituted thiomorpholinyl. In embodiments, R¹ is substituted thianyl. In embodiments, R¹ is substituted oxanyl. In embodiments, R¹ is substituted tetrahydropuranyl. In embodiments, R¹ is substituted dihydropuranyl. In embodiments, R¹ is substituted dioxanyl. In embodiments, R¹ is substituted pyrazolyl. In embodiments, R¹ is substituted pyrrolyl. In embodiments, R¹ is substituted thienyl. In embodiments, R¹ is substituted benzofuranyl. In embodiments, R¹ is substituted indolyl. In embodiments, R¹ is substituted benzothienyl. In embodiments, R¹ is substituted benzimidazolyl. In embodiments, R¹ is substituted isobenzofuranyl. In embodiments, R¹ is substituted isoindolyl. In embodiments, R¹ is substituted benzo[c]thienyl. In embodiments, R¹ is substituted purinyl. In embodiments, R¹ is substituted indazolyl. In embodiments, R¹ is substituted benzoxazolyl. In embodiments, R¹ is substituted benzisoxazolyl. In embodiments, R¹ is substituted benzothiazolyl. In embodiments, R¹ is substituted cyclopentyl. In embodiments, R¹ is substituted cyclobutyl. In embodiments, R¹ is substituted naphthyl. In embodiments, R¹ is substituted 1-naphthyl. In embodiments, R¹ is substituted 2-naphthyl. In embodiments, R¹ is hydrogen. In embodiments, R¹ is substituted 2-thienyl. In embodiments, R¹ is substituted 3-thienyl. In embodiments, R¹ is substituted 2-furanyl. In embodiments, R¹ is substituted 3-furanyl. In embodiments, R¹ is substituted 2-pyridyl. In embodiments, R¹ is substituted 3-pyridyl. In embodiments, R¹ is substituted 4-pyridyl. In embodiments, R¹ is substituted 3-pyrazolyl. In embodiments, R¹ is substituted 4-pyrazolyl. In embodiments, R¹ is substituted 5-pyrazolyl. In embodiments, R¹ is substituted 2-pyrrolyl. In embodiments, R¹ is substituted 3-pyrrolyl.

In embodiments, R¹ is R²⁰-substituted phenyl. In embodiments, R¹ is R²⁰-substituted pyridyl. In embodiments, R¹ is R²⁰-substituted cyclohexyl. In embodiments, R¹ is R²⁰-substituted morpholinyl. In embodiments, R¹ is R²⁰-substituted piperazinyl. In embodiments, R¹ is R²⁰-substituted furanyl. In embodiments, R¹ is R²⁰-substituted thiazolyl. In embodiments, R¹ is R²⁰-substituted pyrazolyl. In embodiments, R¹ is R²⁰-substituted thienyl. In embodiments, R¹ is R²⁰-substituted pyrazinyl. In embodiments, R¹ is R²⁰-substituted pyrimidinyl. In embodiments, R¹ is R²⁰-substituted pyridazinyl. In embodiments, R¹ is R²⁰-substituted triazinyl. In embodiments, R¹ is R²⁰-substituted tetrazinyl. In embodiments, R¹ is R²⁰-substituted tetrazolyl. In embodiments, R¹ is R²⁰-substituted triazolyl. In embodiments, R¹ is R²⁰-substituted quinolinyl. In embodiments, R¹ is R²⁰-substituted isoquinolinyl. In embodiments, R¹ is R²⁰-substituted quinazolinyl. In embodiments, R¹ is R²⁰-substituted quinoxalinyl. In embodiments, R¹ is R²⁰-substituted imidazolyl. In embodiments, R¹ is R²⁰-substituted oxazolyl. In embodiments, R¹ is R²⁰-substituted isoxazolyl. In embodiments, R¹ is R²⁰-substituted thiazolyl. In embodiments, R¹ is R²⁰-substituted piperidinyl. In embodiments, R¹ is R²⁰-substituted thiomorpholinyl. In embodiments, R¹ is R²⁰-substituted thianyl. In embodiments, R¹ is R²⁰-substituted oxanyl. In embodiments, R¹ is R²⁰-substituted tetrahydropuranyl. In embodiments, R¹ is R²⁰-substituted dihydropuranyl. In embodiments, R¹ is R²⁰-substituted dioxanyl. In embodiments, R¹ is R²⁰-substituted pyrazolyl. In embodiments, R¹ is R²⁰-substituted pyrrolyl. In embodiments, R¹ is R²⁰-substituted thienyl. In embodiments, R¹ is R²⁰-substituted benzofuranyl. In embodiments, R¹ is R²⁰-substituted indolyl. In embodiments, R¹ is R²⁰-substituted benzothienyl. In embodiments, R¹ is R²⁰-substituted benzimidazolyl. In embodiments, R¹ is R²⁰-substituted isobenzofuranyl. In embodiments, R¹ is R²⁰-substituted isoindolyl. In embodiments, R¹ is R²⁰-substituted benzo[c]thienyl. In embodiments, R¹ is R²⁰-substituted purinyl. In embodiments, R¹ is R²⁰-substituted indazolyl. In embodiments, R¹ is R²⁰-substituted benzoxazolyl. In embodiments, R¹ is R²⁰-substituted benzisoxazolyl. In embodiments, R¹ is R²⁰-substituted benzothiazolyl. In embodiments, R¹ is R²⁰-substituted cyclopentyl. In embodiments, R¹ is R²⁰-substituted cyclobutyl. In embodiments, R¹ is R²⁰-substituted naphthyl. In embodiments, R¹ is R²⁰-substituted 1-naphthyl. In embodiments, R¹ is R²⁰-substituted 2-naphthyl. In embodiments, R¹ is R²⁰-substituted 2-thienyl. In embodiments, R¹ is R²⁰-substituted 3-thienyl. In embodiments, R¹ is R²⁰-substituted 2-furanyl. In embodiments, R¹ is R²⁰-substituted 3-furanyl. In embodiments, R¹ is R²⁰-substituted 2-pyridyl. In embodiments, R¹ is R²⁰-substituted 3-pyridyl. In embodiments, R¹ is R²⁰-substituted 4-pyridyl. In embodiments, R¹ is R²⁰-substituted 3-pyrazolyl. In embodiments, R¹ is R²⁰-substituted 4-pyrazolyl. In embodiments, R¹ is R²⁰-substituted 5-pyrazolyl. In embodiments, R¹ is R²⁰-substituted 2-pyrrolyl. In embodiments, R¹ is R²⁰-substituted 3-pyrrolyl.

In embodiments, R¹ is an unsubstituted phenyl. In embodiments, R¹ is an unsubstituted pyridyl. In embodiments, R¹ is an unsubstituted cyclohexyl. In embodiments, R¹ is an unsubstituted morpholinyl. In embodiments, R¹ is an unsubstituted piperazinyl. In embodiments, R¹ is an unsubstituted furanyl. In embodiments, R¹ is an unsubstituted thiazolyl. In embodiments, R¹ is an unsubstituted pyrazolyl. In embodiments, R¹ is an unsubstituted thienyl. In embodiments, R¹ is an unsubstituted pyrazinyl. In embodiments, R¹ is an unsubstituted pyrimidinyl. In embodiments, R¹ is an unsubstituted pyridazinyl. In embodiments, R¹ is an unsubstituted triazinyl. In embodiments, R¹ is an unsubstituted tetrazinyl. In embodiments, R¹ is an unsubstituted tetrazolyl. In embodiments, R¹ is an unsubstituted triazolyl. In embodiments, R¹ is an unsubstituted quinolinyl. In embodiments, R¹ is an unsubstituted isoquinolinyl. In embodiments, R¹ is an unsubstituted quinazolinyl. In embodiments, R¹ is an unsubstituted quinoxalinyl. In embodiments, R¹ is an unsubstituted imidazolyl. In embodiments, R¹ is an unsubstituted oxazolyl. In embodiments, R¹ is an unsubstituted isoxazolyl. In embodiments, R¹ is an unsubstituted thiazolyl. In embodiments, R¹ is an unsubstituted piperidinyl. In embodiments, R¹ is an unsubstituted thiomorpholinyl. In embodiments, R¹ is an unsubstituted thianyl. In embodiments, R¹ is an unsubstituted oxanyl. In embodiments, R¹ is an unsubstituted tetrahydropuranyl. In embodiments, R¹ is an unsubstituted dihydropuranyl. In embodiments, R¹ is an unsubstituted dioxanyl. In embodiments, R¹ is an unsubstituted pyrazolyl. In embodiments, R¹ is an unsubstituted pyrrolyl. In embodiments, R¹ is an unsubstituted thienyl. In embodiments, R¹ is an unsubstituted benzofuranyl. In embodiments, R¹ is an unsubstituted indolyl. In embodiments, R¹ is an unsubstituted benzothienyl. In embodiments, R¹ is an unsubstituted benzimidazolyl. In embodiments, R¹ is an unsubstituted isobenzofuranyl. In embodiments, R¹ is an unsubstituted isoindolyl. In embodiments, R¹ is an unsubstituted benzo[c]thienyl. In embodiments, R¹ is an unsubstituted purinyl. In embodiments, R¹ is an unsubstituted indazolyl. In embodiments, R¹ is an unsubstituted benzoxazolyl. In embodiments, R¹ is an unsubstituted benzisoxazolyl. In embodiments, R¹ is an unsubstituted benzothiazolyl. In embodiments, R¹ is an unsubstituted cyclopentyl. In embodiments, R¹ is an unsubstituted cyclobutyl. In embodiments, R¹ is an unsubstituted naphthyl. In embodiments, R¹ is an unsubstituted 1-naphthyl. In embodiments, R¹ is an unsubstituted 2-naphthyl. In embodiments, R¹ is an unsubstituted 2-thienyl. In embodiments, R¹ is an unsubstituted 3-thienyl. In embodiments, R¹ is an unsubstituted 2-furanyl. In embodiments, R¹ is an unsubstituted 3-furanyl. In embodiments, R¹ is an unsubstituted 2-pyridyl. In embodiments, R¹ is an unsubstituted 3-pyridyl. In embodiments, R¹ is an unsubstituted 4-pyridyl. In embodiments, R¹ is an unsubstituted 3-pyrazolyl. In embodiments, R¹ is an unsubstituted 4-pyrazolyl. In embodiments, R¹ is an unsubstituted 5-pyrazolyl. In embodiments, R¹ is an unsubstituted 2-pyrrolyl. In embodiments, R¹ is an unsubstituted 3-pyrrolyl.

In embodiments, R¹ is substituted or unsubstituted aryl or substituted or unsubstituted heteroaryl. In embodiments, R¹ is substituted aryl. In embodiments, R¹ is an unsubstituted aryl. In embodiments, R¹ is substituted C₆-C₁₀ aryl. In embodiments, R¹ is an unsubstituted C₆-C₁₀ aryl. In embodiments, R¹ is substituted phenyl. In embodiments, R¹ is an unsubstituted phenyl. In embodiments, R¹ is substituted heteroaryl. In embodiments, R¹ is an unsubstituted heteroaryl. In embodiments, R¹ is substituted 5 to 10 membered heteroaryl. In embodiments, R¹ is substituted 5 to 9 membered heteroaryl. In embodiments, R¹ is an unsubstituted 5 to 10 membered heteroaryl. In embodiments, R¹ is an unsubstituted 5 to 9 membered heteroaryl. In embodiments, R¹ is substituted 5 to 6 membered heteroaryl. In embodiments, R¹ is an unsubstituted 5 to 6 membered heteroaryl. In embodiments, R¹ is substituted 9 membered heteroaryl. In embodiments, R¹ is substituted 10 membered heteroaryl. In embodiments, R¹ is an unsubstituted 9 membered heteroaryl. In embodiments, R¹ is an unsubstituted 10 membered heteroaryl. In embodiments, R¹ is substituted 5 membered heteroaryl. In embodiments, R¹ is substituted 6 membered heteroaryl. In embodiments, R¹ is an unsubstituted 5 membered heteroaryl. In embodiments, R¹ is an unsubstituted 6 membered heteroaryl.

In embodiments, R¹ is substituted or unsubstituted pyrazolyl. In embodiments, R¹ is substituted or unsubstituted pyridyl. In embodiments, R¹ is substituted or unsubstituted imidazolyl. In embodiments, R¹ is substituted or unsubstituted oxazolyl. In embodiments, R¹ is substituted or unsubstituted isoxazolyl. In embodiments, R¹ is substituted or unsubstituted thiazolyl. In embodiments, R¹ is substituted or unsubstituted furanyl. In embodiments, R¹ is substituted or unsubstituted pyrrolyl. In embodiments, R¹ is substituted or unsubstituted thienyl. In embodiments, R¹ is substituted pyrazolyl. In embodiments, R¹ is substituted pyridyl. In embodiments, R¹ is substituted imidazolyl. In embodiments, R¹ is substituted oxazolyl. In embodiments, R¹ is substituted isoxazolyl. In embodiments, R¹ is substituted thiazolyl. In embodiments, R¹ is substituted furanyl. In embodiments, R¹ is substituted pyrrolyl. In embodiments, R¹ is substituted thienyl.

In embodiments, R¹ is an unsubstituted pyrazolyl. In embodiments, R¹ is an unsubstituted pyridyl. In embodiments, R¹ is an unsubstituted imidazolyl. In embodiments, R¹ is an unsubstituted oxazolyl. In embodiments, R¹ is an unsubstituted isoxazolyl. In embodiments, R¹ is an unsubstituted thiazolyl. In embodiments, R¹ is an unsubstituted furanyl. In embodiments, R¹ is an unsubstituted pyrrolyl. In embodiments, R¹ is an unsubstituted thienyl.

In embodiments, R¹ is a methyl-substituted pyrazolyl. In embodiments, R¹ is a methyl-substituted pyridyl. In embodiments, R¹ is methyl-substituted imidazolyl. In embodiments, R¹ is a methyl-substituted oxazolyl. In embodiments, R¹ is a methyl-substituted isoxazolyl. In embodiments, R¹ is a methyl-substituted thiazolyl. In embodiments, R¹ is a methyl-substituted furanyl. In embodiments, R¹ is a methyl-substituted pyrrolyl. In embodiments, R¹ is a methyl-substituted thienyl.

In embodiments, R¹ is independently R²⁰-substituted or unsubstituted aryl or R²⁰-substituted or unsubstituted heteroaryl. In embodiments, R¹ is independently R²⁰-substituted or unsubstituted phenyl or R²⁰-substituted or unsubstituted 5 to 6 membered heteroaryl. X¹ is —F, —Cl, —Br, or —I.

In embodiments, R²⁰ is —C(O)CH₃. In embodiments, R²⁰ is —CH₃. In embodiments, R²⁰ is —C(O)CH₂CH₃. In embodiments, R²⁰ is —C(O)CH(CH₃)₂. In embodiments, R²⁰ is an unsubstituted methyl. In embodiments, R²⁰ is —C(O)N(CH₃)₂. In embodiments, R²⁰ is —CN. In embodiments, R²⁰ is an unsubstituted methoxy. In embodiments, R²⁰ is an unsubstituted tert-butyl. In embodiments, R²⁰ is —OH. In embodiments, R²⁰ is an unsubstituted ethoxy. In embodiments, R²⁰ is —N(CH₃)₂. In embodiments, R²⁰ is —SH. In embodiments, R²⁰ is —SCH₃. In embodiments, R²⁰ is —SCH₂CH₃. In embodiments, R²⁰ is an unsubstituted ethyl. In embodiments, R²⁰ is an unsubstituted propyl. In embodiments, R²⁰ is an unsubstituted isopropyl. In embodiments, R²⁰ is an unsubstituted butyl. In embodiments, R²⁰ is an unsubstituted isobutyl. In embodiments, R²⁰ is —NH₂. In embodiments, R²⁰ is —NHCH₃. In embodiments, R²⁰ is —NHCH₂CH₃. In embodiments, R²⁰ is —N(CH₂CH₃)₂. In embodiments, R²⁰ is —N(CH₃)(CH₂CH₃). In embodiments, R²⁰ is halogen. In embodiments, R²⁰ is —F. In embodiments, R²⁰ is —Cl. In embodiments, R²⁰ is —I. In embodiments, R²⁰ is —Br. In embodiments, R²⁰ is —C(O)NH₂. In embodiments, R²⁰ is —C(O)NHCH₃. In embodiments, R²⁰ is —C(O)NHCH₂CH₃. In embodiments, R²⁰ is —C(O)N(CH₂CH₃)₂. In embodiments, R²⁰ is —C(O)N(CH₃)(CH₂CH₃). In embodiments, R²⁰ is independently oxo. In embodiments, R²⁰ is independently halogen. In embodiments, R²⁰ is independently —CX²⁰₃. In embodiments, R²⁰ is independently —CN. In embodiments, R²⁰ is independently —OH. In embodiments, R²⁰ is independently —NH₂. In embodiments, R²⁰ is independently —COOH. In embodiments, R²⁰ is independently —CONH₂. In embodiments, R²⁰ is independently —NO₂. In embodiments, R²⁰ is independently —SH. In embodiments, R²⁰ is independently —SO₃H. In embodiments, R²⁰ is independently —SO₄H. In embodiments, R²⁰ is independently —SO₂NH₂. In embodiments, R²⁰ is independently —NHNH₂. In embodiments, R²⁰ is independently —ONH₂. In embodiments, R²⁰ is independently —NHC(O)NHNH₂. In embodiments, R²⁰ is independently —NHC(O)NH₂. In embodiments, R²⁰ is independently —NHSO₂H. In embodiments, R²⁰ is independently —NHC(O)H. In embodiments, R²⁰ is independently —NHC(O)OH. In embodiments, R²⁰ is independently —NHOH. In embodiments, R²⁰ is independently —OCX²⁰₃. In embodiments, R²⁰ is independently —OCHX²⁰₂. In embodiments, R²⁰ is independently —CF₃.

R²⁰ is independently oxo, halogen, —CX²⁰₃, —CHX²⁰₂, —CH₂X²⁰, —OCX²⁰₃, —OCHX²⁰₂, —OCH₂X²⁰, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²¹-substituted or unsubstituted alkyl (e.g., C₁-C₈, C₁-C₆, or C₁-C₄), R²¹-substituted or unsubstituted heteroalkyl (e.g., 2 to 8 membered, 2 to 6 membered, or 2 to 4 membered), R²¹-substituted or unsubstituted cycloalkyl (e.g., C₃-C₈, C₃-C₆, or C₅-C₆), R²¹-substituted or unsubstituted heterocycloalkyl (e.g., 3 to 8 membered, 3 to 6 membered, or 5 to 6 membered), R²¹-substituted or unsubstituted aryl (e.g., C₆-C₁₀, C₁₀, or phenyl), or R²¹-substituted or unsubstituted heteroaryl (e.g., 5 to 10 membered, 5 to 9 membered, or 5 to 6 membered). X²⁰ is —F, —Cl, —Br, or —I.

R²¹ is independently oxo, halogen, —CX²¹₃, —CHX²¹₂, —CH₂X²¹, —OCX²¹₃, —OCHX²¹₂, —OCH₂X²¹, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²²-substituted or unsubstituted alkyl (e.g., C₁-C₈, C₁-C₆, or C₁-C₄), R²²-substituted or unsubstituted heteroalkyl (e.g., 2 to 8 membered, 2 to 6 membered, or 2 to 4 membered), R²²-substituted or unsubstituted cycloalkyl (e.g., C₃-C₈, C₃-C₆, or C₅-C₆), R²²-substituted or unsubstituted heterocycloalkyl (e.g., 3 to 8 membered, 3 to 6 membered, or 5 to 6 membered), R²²-substituted or unsubstituted aryl (e.g., C₆-C₁₀, C₁₀, or phenyl), or R²²-substituted or unsubstituted heteroaryl (e.g., 5 to 10 membered, 5 to 9 membered, or 5 to 6 membered). X²¹ is —F, —Cl, —Br, or —I.

R²² is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, R¹ is R²⁰-substituted or unsubstituted aryl or R²⁰-substituted or unsubstituted heteroaryl. In embodiments, R¹ is R²⁰-substituted aryl. In embodiments, R¹ is an unsubstituted aryl. In embodiments, R¹ is R²⁰-substituted C₆-C₁₀ aryl. In embodiments, R¹ is an unsubstituted C₆-C₁₀ aryl. In embodiments, R¹ is R²⁰-substituted phenyl. In embodiments, R¹ is an unsubstituted phenyl. In embodiments, R¹ is R²⁰-substituted heteroaryl. In embodiments, R¹ is an unsubstituted heteroaryl. In embodiments, R¹ is R²⁰-substituted 5 to 10 membered heteroaryl. In embodiments, R¹ is R²⁰-substituted 5 to 9 membered heteroaryl. In embodiments, R¹ is an unsubstituted 5 to 10 membered heteroaryl. In embodiments, R¹ is an unsubstituted 5 to 9 membered heteroaryl. In embodiments, R¹ is R²⁰-substituted 5 to 6 membered heteroaryl. In embodiments, R¹ is an unsubstituted 5 to 6 membered heteroaryl. In embodiments, R¹ is R²⁰-substituted 9 membered heteroaryl. In embodiments, R¹ is R²⁰-substituted 10 membered heteroaryl. In embodiments, R¹ is an unsubstituted 9 membered heteroaryl. In embodiments, R¹ is an unsubstituted 10 membered heteroaryl. In embodiments, R¹ is R²⁰-substituted 5 membered heteroaryl. In embodiments, R¹ is R²⁰-substituted 6 membered heteroaryl. In embodiments, R¹ is an unsubstituted 5 membered heteroaryl. In embodiments, R¹ is an unsubstituted 6 membered heteroaryl.

In embodiments, R¹ is R²⁰-substituted or unsubstituted pyrazolyl. In embodiments, R¹ is R²⁰-substituted or unsubstituted pyridyl. In embodiments, R¹ is R²⁰-substituted or unsubstituted imidazolyl. In embodiments, R¹ is R²⁰-substituted or unsubstituted oxazolyl. In embodiments, R¹ is R²⁰-substituted or unsubstituted isoxazolyl. In embodiments, R¹ is R²⁰-substituted or unsubstituted thiazolyl. In embodiments, R¹ is R²⁰-substituted or unsubstituted furanyl. In embodiments, R¹ is R²⁰-substituted or unsubstituted pyrrolyl. In embodiments, R¹ is R²⁰-substituted or unsubstituted thienyl.

In embodiments, R¹ is R²⁰-substituted pyrazolyl. In embodiments, R¹ is R²⁰-substituted pyridyl. In embodiments, R¹ is R²⁰-substituted imidazolyl. In embodiments, R¹ is R²⁰-substituted oxazolyl. In embodiments, R¹ is R²⁰-substituted isoxazolyl. In embodiments, R¹ is R²⁰-substituted thiazolyl. In embodiments, R¹ is R²⁰-substituted furanyl. In embodiments, R¹ is R²⁰-substituted pyrrolyl. In embodiments, R¹ is R²⁰-substituted thienyl. In embodiments, R¹ is an unsubstituted pyrazolyl. In embodiments, R¹ is an unsubstituted pyridyl. In embodiments, R¹ is an unsubstituted imidazolyl. In embodiments, R¹ is an unsubstituted oxazolyl. In embodiments, R¹ is an unsubstituted isoxazolyl. In embodiments, R¹ is an unsubstituted thiazolyl. In embodiments, R¹ is an unsubstituted furanyl. In embodiments, R¹ is an unsubstituted pyrrolyl. In embodiments, R¹ is an unsubstituted thienyl.

R² is hydrogen, —CX²₃, —CHX²₂, —CH₂X², substituted or unsubstituted alkyl, or substituted or unsubstituted heteroalkyl. In embodiments, R² is —CF₃, —CCl₃, —CBr₃, —CI₃, —CHF₂, —CHCl₂, —CHBr₂, —CHI₂, —CH₂F, —CH₂Cl, —CH₂Br, —CH₂I.

In embodiments, R² is independently hydrogen, —CX²₃, —CHX²₂, —CH₂X², substituted or unsubstituted C₁-C₈ alkyl, or substituted or unsubstituted 2 to 8 membered heteroalkyl. In embodiments, R² is hydrogen, —CX²₃, —CHX²₂, —CH₂X², unsubstituted C₁-C₄ alkyl, or unsubstituted 2 to 4 membered heteroalkyl. In embodiments, R² is independently hydrogen, —CX²₃, —CHX²₂, —CH₂X², unsubstituted methyl, unsubstituted ethyl, unsubstituted methoxy, or unsubstituted ethoxy. In embodiments, R² is independently hydrogen. In embodiments, R² is independently unsubstituted methyl. In embodiments, R² is independently unsubstituted ethyl. In embodiments, R² is independently unsubstituted propyl. In embodiments, R² is independently unsubstituted n-propyl. In embodiments, R² is independently unsubstituted isopropyl. In embodiments, R² is independently unsubstituted butyl. In embodiments, R² is independently unsubstituted n-butyl. In embodiments, R² is independently unsubstituted isobutyl. In embodiments, R² is independently unsubstituted tert-butyl. In embodiments, R² is independently unsubstituted pentyl. In embodiments, R² is independently unsubstituted hexyl. In embodiments, R² is independently unsubstituted heptyl. In embodiments, R² is independently unsubstituted octyl. In embodiments, X² is independently —F. In embodiments, X² is independently —Cl. In embodiments, X² is independently —Br. In embodiments, X² is independently —I. In embodiments, R² is independently unsubstituted methoxy. In embodiments, R² is independently unsubstituted ethoxy. In embodiments, R² is independently —CF₃. In embodiments, R² is independently —CCl₃.

In embodiments, R² is independently —CX²₃. In embodiments, R² is independently —CHX²₂. In embodiments, R² is independently —CH₂X².

In embodiments, R² is independently substituted or unsubstituted alkyl. In embodiments, R² is independently substituted or unsubstituted heteroalkyl. In embodiments, R² is independently substituted alkyl. In embodiments, R² is independently substituted heteroalkyl. In embodiments, R² is independently unsubstituted alkyl. In embodiments, R² is independently unsubstituted heteroalkyl. In embodiments, R² is independently substituted or unsubstituted C₁-C₈ alkyl. In embodiments, R² is independently substituted or unsubstituted 2 to 8 membered heteroalkyl. In embodiments, R² is independently substituted C₁-C₈ alkyl. In embodiments, R² is independently substituted 2 to 8 membered heteroalkyl. In embodiments, R² is independently unsubstituted C₁-C₈ alkyl. In embodiments, R² is independently unsubstituted 2 to 8 membered heteroalkyl. In embodiments, R² is independently substituted or unsubstituted C₁-C₄ alkyl. In embodiments, R² is independently substituted or unsubstituted 2 to 4 membered heteroalkyl. In embodiments, R² is independently substituted C₁-C₄ alkyl. In embodiments, R² is independently substituted 2 to 4 membered heteroalkyl. In embodiments, R² is independently unsubstituted C₁-C₄ alkyl. In embodiments, R² is independently unsubstituted 2 to 4 membered heteroalkyl.

In embodiments, R² is independently hydrogen, —CX²₃, —CHX²₂, —CH₂X², R²³-substituted or unsubstituted alkyl, or R²³-substituted or unsubstituted heteroalkyl. In embodiments, R² is independently hydrogen, —CX²₃, —CHX²₂, —CH₂X², R²³-substituted or unsubstituted C₁-C₈ alkyl, or R²³-substituted or unsubstituted 2 to 8 membered heteroalkyl. X² is —F, —Cl, —Br, or —I. In embodiments, R² is independently hydrogen. In embodiments, R² is independently methyl. In embodiments, R² is independently ethyl.

R²³ is independently oxo, halogen, —CX²³₃, —CHX²³₂, —CH₂X²³, —OCX²³₃, —OCHX²³₂, —OCH₂X²³, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²⁴-substituted or unsubstituted alkyl (e.g., C₁-C₈, C₁-C₆, or C₁-C₄), R²⁴-substituted or unsubstituted heteroalkyl (e.g., 2 to 8 membered, 2 to 6 membered, or 2 to 4 membered), R²⁴-substituted or unsubstituted cycloalkyl (e.g., C₃-C₈, C₃-C₆, or C₅-C₆), R²⁴-substituted or unsubstituted heterocycloalkyl (e.g., 3 to 8 membered, 3 to 6 membered, or 5 to 6 membered), R²⁴-substituted or unsubstituted aryl (e.g., C₆-C₁₀, C₁₀, or phenyl), or R²⁴-substituted or unsubstituted heteroaryl (e.g., 5 to 10 membered, 5 to 9 membered, or 5 to 6 membered). X²³ is —F, —Cl, —Br, or —I.

R²⁴ is independently oxo, halogen, —CX²⁴₃, —CHX²⁴₂, —CH₂X²⁴, —OCX²⁴₃, —OCHX²⁴₂, —OCH₂X²⁴, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²⁵-substituted or unsubstituted alkyl (e.g., C₁-C₈, C₁-C₆, or C₁-C₄), R²⁵-substituted or unsubstituted heteroalkyl (e.g., 2 to 8 membered, 2 to 6 membered, or 2 to 4 membered), R²⁵-substituted or unsubstituted cycloalkyl (e.g., C₃-C₈, C₃-C₆, or C₅-C₆), R²⁵-substituted or unsubstituted heterocycloalkyl (e.g., 3 to 8 membered, 3 to 6 membered, or 5 to 6 membered), R²⁵-substituted or unsubstituted aryl (e.g., C₆-C₁₀, C₁₀, or phenyl), or R²⁵-substituted or unsubstituted heteroaryl (e.g., 5 to 10 membered, 5 to 9 membered, or 5 to 6 membered). X²⁴ is —F, —Cl, —Br, or —I.

R²⁵ is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, R² is independently hydrogen. In embodiments, R² is independently substituted or unsubstituted methyl. In embodiments, R² is independently substituted or unsubstituted C₁-C₄ alkyl. In embodiments, R² is independently substituted methyl. In embodiments, R² is independently substituted C₁-C₄ alkyl. In embodiments, R² is independently unsubstituted methyl. In embodiments, R² is independently unsubstituted C₁-C₄ alkyl.

In embodiments, R² is independently substituted or unsubstituted C₁-C₄ alkyl. In embodiments, R² is independently substituted or unsubstituted C₁-C₃ alkyl. In embodiments, R² is independently substituted or unsubstituted C₁-C₂ alkyl. In embodiments, R² is independently substituted or unsubstituted methyl. In embodiments, R² is independently substituted or unsubstituted methyl or substituted or unsubstituted isopropyl.

In embodiments, R³ is independently unsubstituted heteroalkyl. In embodiments, R³ is independently unsubstituted 2 to 5 membered heteroalkyl. In embodiments, R³ is independently —OCH₃, —OCH₂CH₃, —N(CH₃)₂, —NH₂, —NH(CH₃), —N(CH₂CH₃)₂, —NH(CH₂CH₃), or —SH. In embodiments, R³ is independently OCH₃. In embodiments, R³ is independently —OCH₂CH₃. In embodiments, R³ is independently —N(CH₃)₂. In embodiments, R³ is independently —NH₂. In embodiments, R³ is independently —NH(CH₃). In embodiments, R³ is independently —N(CH₂CH₃)₂. In embodiments, R³ is independently —NH(CH₂CH₃). In embodiments, R³ is independently —SH. In embodiments, R³ is independently —OCH₂CH₂CH₃. In embodiments, R³ is independently unsubstituted methoxy. In embodiments, R³ is independently unsubstituted ethoxy. In embodiments, R³ is independently unsubstituted propoxy. In embodiments, R³ is independently unsubstituted isopropoxy. In embodiments, R³ is independently unsubstituted butoxy. In embodiments, R³ is independently unsubstituted tert-butoxy. In embodiments, R³ is independently unsubstituted pentoxy. In embodiments, R³ is independently unsubstituted hexoxy.

In embodiments, R³ is an unsubstituted methoxy. In embodiments, R³ is —OCHF₂. In embodiments, R³ is an unsubstituted tert-butyl. In embodiments, R³ is an unsubstituted phenoxy. In embodiments, R³ is an unsubstituted methyl. In embodiments, R³ is —OH. In embodiments, R³ is an unsubstituted ethoxy. In embodiments, R³ is —N(CH₃)₂. In embodiments, R³ is —SH. In embodiments, R³ is —SCH₃. In embodiments, R³ is —SCH₂CH₃. In embodiments, R³ is an unsubstituted ethyl. In embodiments, R³ is an unsubstituted propyl. In embodiments, R³ is an unsubstituted isopropyl. In embodiments, R³ is an unsubstituted butyl. In embodiments, R³ is an unsubstituted isobutyl. In embodiments, R³ is —NH₂. In embodiments, R³ is —NHCH₃. In embodiments, R³ is —NHCH₂CH₃. In embodiments, R³ is —N(CH₂CH₃)₂. In embodiments, R³ is —N(CH₃)(CH₂CH₃). In embodiments, R³ is halogen. In embodiments, R³ is —F. In embodiments, R³ is —Cl. In embodiments, R³ is —I. In embodiments, R³ is —Br. In embodiments, R³ is independently —CF₃. In embodiments, R³ is independently —OCH₃. In embodiments, R³ is an unsubstituted phenyl. In embodiments, R³ is independently —C(O)N(CH₃)₂. In embodiments, R³ is independently —C(O)NH(CH₃). In embodiments, R³ is independently —C(O)N(CH₂CH₃)₂. In embodiments, R³ is independently —C(O)NH(CH₂CH₃).

In embodiments, R³ is independently unsubstituted cyclohexyl. In embodiments, R³ is independently unsubstituted morpholinyl. In embodiments, R³ is independently unsubstituted piperazinyl. In embodiments, R³ is independently N-methyl substituted piperazinyl. In embodiments, R³ is independently unsubstituted pyridyl. In embodiments, R³ is an unsubstituted cyclopentyl. In embodiments, R³ is an unsubstituted cyclobutyl. In embodiments, R³ is an unsubstituted naphthyl. In embodiments, R³ is an unsubstituted 1-naphthyl. In embodiments, R³ is an unsubstituted 2-naphthyl. In embodiments, R³ is an unsubstituted 2-thienyl. In embodiments, R³ is an unsubstituted 3-thienyl. In embodiments, R³ is an unsubstituted 2-furanyl. In embodiments, R³ is an unsubstituted 3-furanyl. In embodiments, R³ is an unsubstituted 2-pyridyl. In embodiments, R³ is an unsubstituted 3-pyridyl. In embodiments, R³ is an unsubstituted 4-pyridyl. In embodiments, R³ is an unsubstituted 3-pyrazolyl. In embodiments, R³ is an unsubstituted 4-pyrazolyl. In embodiments, R³ is an unsubstituted 5-pyrazolyl. In embodiments, R³ is an unsubstituted 2-pyrrolyl. In embodiments, R³ is an unsubstituted 3-pyrrolyl. In embodiments, R³ is an unsubstituted 2-thiazolyl. In embodiments, R³ is an unsubstituted 4-thiazolyl. In embodiments, R³ is an unsubstituted 5-thiazolyl. In embodiments, R³ is an unsubstituted thiazolyl. In embodiments, R³ is substituted thiazolyl. In embodiments, R³ is methyl substituted thiazolyl. In embodiments, R³ is an unsubstituted thienyl. In embodiments, R³ is substituted thienyl. In embodiments, R³ is methyl substituted thienyl. In embodiments, R³ is an unsubstituted pyrazolyl. In embodiments, R³ is substituted pyrazolyl. In embodiments, R³ is methyl substituted pyrazolyl. In embodiments, R³ is an unsubstituted furanyl. In embodiments, R³ is substituted furanyl. In embodiments, R³ is methyl substituted furanyl.

In embodiments, R³ is independently halogen. In embodiments, R³ is independently —CX³₃. In embodiments, R³ is independently —CHX³₂. In embodiments, R³ is independently —CH₂X³. In embodiments, R³ is independently —OCX³₃. In embodiments, R³ is independently —OCH₂X³. In embodiments, R³ is independently —OCHX³₂. In embodiments, R³ is independently —CN. In embodiments, R³ is independently —SOₙ₃R^{3D}. In embodiments, R³ is independently —SOᵥ₃NR^{3A}R^{3B}. In embodiments, R³ is independently —NHC(O)NR^{3A}R^{3B}. In embodiments, R³ is independently —N(O)ₘ₃. In embodiments, R³ is independently —NR^{3A}R^{3B}. In embodiments, R³ is independently —C(O)R^{3C}. In embodiments, R³ is independently —C(O)—OR^{3C}. In embodiments, R³ is independently —C(O)NR^{3A}R^{3B}. In embodiments, R³ is independently —OR^{3D}. In embodiments, R³ is independently —NR^{3A}SO₂R^{3D}. In embodiments, R³ is independently —NR^{3A}C(O)R^{3C}. In embodiments, R³ is independently —NR^{3A}C(O)OR^{3C}. In embodiments, R³ is independently —NR^{3A}OR^{3C}. In embodiments, R³ is independently —OH. In embodiments, R³ is independently —NH₂. In embodiments, R³ is independently —COOH. In embodiments, R³ is independently —CONH₂. In embodiments, R³ is independently —NO₂. In embodiments, R³ is independently —SH.

In embodiments, R³ is independently substituted or unsubstituted alkyl (e.g., C₁-C₈, C₁-C₆, or C₁-C₄). In embodiments, R³ is independently substituted alkyl (e.g., C₁-C₈, C₁-C₆, or C₁-C₄). In embodiments, R³ is independently unsubstituted alkyl (e.g., C₁-C₈, C₁-C₆, or C₁-C₄). In embodiments, R³ is independently substituted or unsubstituted heteroalkyl (e.g., 2 to 8 membered, 2 to 6 membered, or 2 to 4 membered). In embodiments, R³ is independently substituted heteroalkyl (e.g., 2 to 8 membered, 2 to 6 membered, or 2 to 4 membered). In embodiments, R³ is independently unsubstituted heteroalkyl (e.g., 2 to 8 membered, 2 to 6 membered, or 2 to 4 membered). In embodiments, R³ is independently substituted or unsubstituted cycloalkyl (e.g., C₃-C₈, C₃-C₆, or C₅-C₆). In embodiments, R³ is independently substituted cycloalkyl (e.g., C₃-C₈, C₃-C₆, or C₅-C₆). In embodiments, R³ is independently unsubstituted cycloalkyl (e.g., C₃-C₈, C₃-C₆, or C₅-C₆).

In embodiments, R³ is independently substituted or unsubstituted heterocycloalkyl (e.g., 3 to 8 membered, 3 to 6 membered, or 5 to 6 membered). In embodiments, R³ is independently substituted heterocycloalkyl (e.g., 3 to 8 membered, 3 to 6 membered, or 5 to 6 membered). In embodiments, R³ is independently unsubstituted heterocycloalkyl (e.g., 3 to 8 membered, 3 to 6 membered, or 5 to 6 membered). In embodiments, R³ is independently substituted or unsubstituted aryl (e.g., C₆-C₁₀, C₁₀, or phenyl). In embodiments, R³ is independently substituted aryl (e.g., C₆-C₁₀, C₁₀, or phenyl). In embodiments, R³ is independently unsubstituted aryl (e.g., C₆-C₁₀, C₁₀, or phenyl). In embodiments, R³ is independently substituted or unsubstituted heteroaryl (e.g., 5 to 10 membered, 5 to 9 membered, or 5 to 6 membered). In embodiments, R³ is independently substituted heteroaryl (e.g., 5 to 10 membered, 5 to 9 membered, or 5 to 6 membered). In embodiments, R³ is independently unsubstituted heteroaryl (e.g., 5 to 10 membered, 5 to 9 membered, or 5 to 6 membered).

In embodiments, R³ is substituted or unsubstituted a cycloalkyl. In embodiments, R³ is substituted or unsubstituted C₃-C₈ cycloalkyl. In embodiments, R³ is substituted or unsubstituted C₃-C₇ cycloalkyl. In embodiments, R³ is substituted or unsubstituted C₃-C₆ cycloalkyl. In embodiments, R³ is substituted or unsubstituted C₃-C₅ cycloalkyl. In embodiments, R³ is substituted or unsubstituted C₃-C₄ cycloalkyl. In embodiments, R³ is substituted or unsubstituted C₄-C₈ cycloalkyl. In embodiments, R³ is substituted or unsubstituted C₅-C₈ cycloalkyl. In embodiments, R³ is substituted or unsubstituted C₆-C₈ cycloalkyl. In embodiments, R³ is substituted or unsubstituted C₅-C₆ cycloalkyl. In embodiments, R³ is substituted or unsubstituted C₃ cycloalkyl. In embodiments, R³ is substituted or unsubstituted C₄ cycloalkyl. In embodiments, R³ is substituted or unsubstituted C₅ cycloalkyl. In embodiments, R³ is substituted or unsubstituted C₆ cycloalkyl. In embodiments, R³ is substituted or unsubstituted C₇ cycloalkyl. In embodiments, R³ is substituted or unsubstituted C₈ cycloalkyl. In embodiments, R³ is substituted or unsubstituted cyclopropyl. In embodiments, R³ is substituted or unsubstituted cyclobutyl. In embodiments, R³ is substituted or unsubstituted cyclopentyl. In embodiments, R³ is substituted or unsubstituted cyclohexyl. In embodiments, R³ is substituted or unsubstituted cycloheptyl. In embodiments, R³ is substituted or unsubstituted a heterocycloalkyl. In embodiments, R³ is substituted or unsubstituted 3 to 8 membered heterocycloalkyl. In embodiments, R³ is substituted or unsubstituted 3 to 7 membered heterocycloalkyl. In embodiments, R³ is substituted or unsubstituted 3 to 6 membered heterocycloalkyl. In embodiments, R³ is substituted or unsubstituted 3 to 5 membered heterocycloalkyl. In embodiments, R³ is substituted or unsubstituted 3 to 4 membered heterocycloalkyl. In embodiments, R³ is substituted or unsubstituted 4 to 8 membered heterocycloalkyl. In embodiments, R³ is substituted or unsubstituted 5 to 8 membered heterocycloalkyl. In embodiments, R³ is substituted or unsubstituted 6 to 8 membered heterocycloalkyl. In embodiments, R³ is substituted or unsubstituted 5 to 6 membered heterocycloalkyl. In embodiments, R³ is substituted or unsubstituted 3 membered heterocycloalkyl. In embodiments, R³ is substituted or unsubstituted 4 membered heterocycloalkyl. In embodiments, R³ is substituted or unsubstituted 5 membered heterocycloalkyl. In embodiments, R³ is substituted or unsubstituted 6 membered heterocycloalkyl. In embodiments, R³ is substituted or unsubstituted 7 membered heterocycloalkyl. In embodiments, R³ is substituted or unsubstituted 8 membered heterocycloalkyl.

In embodiments, R³ is substituted or unsubstituted aryl or heteroaryl. In embodiments, R³ is substituted or unsubstituted aryl. In embodiments, R³ is substituted or unsubstituted heteroaryl. In embodiments, R³ is substituted or unsubstituted C₆-C₁₀ aryl. In embodiments, R³ is substituted or unsubstituted C₁₀ aryl. In embodiments, R³ is substituted or unsubstituted phenyl. In embodiments, R³ is substituted or unsubstituted 5 to 10 membered heteroaryl. In embodiments, R³ is substituted or unsubstituted 5 to 9 membered heteroaryl. In embodiments, R³ is substituted or unsubstituted 5 to 6 membered heteroaryl. In embodiments, R³ is substituted or unsubstituted 10 membered heteroaryl. In embodiments, R³ is substituted or unsubstituted 9 membered heteroaryl. In embodiments, R³ is substituted or unsubstituted 5 membered heteroaryl. In embodiments, R³ is substituted or unsubstituted 6 membered heteroaryl. In embodiments, R³ is substituted or unsubstituted phenyl. In embodiments, R³ is substituted or unsubstituted pyridyl. In embodiments, R³ is substituted or unsubstituted pyrazolyl. In embodiments, R³ is substituted or unsubstituted imidazolyl. In embodiments, R³ is substituted or unsubstituted oxazolyl. In embodiments, R³ is substituted or unsubstituted isoxazolyl. In embodiments, R³ is substituted or unsubstituted thiazolyl. In embodiments, R³ is substituted or unsubstituted furanyl. In embodiments, R³ is substituted or unsubstituted pyrrolyl. In embodiments, R³ is substituted or unsubstituted thienyl. In embodiments, R³ is a two fused ring aryl. In embodiments, R³ is a two fused ring heteroaryl.

In embodiments, R³ is substituted or unsubstituted aryl or heteroaryl. In embodiments, R³ is substituted or unsubstituted phenyl or 5 to 6 membered heteroaryl. In embodiments, R³ is substituted or unsubstituted phenyl. In embodiments, R³ is substituted or unsubstituted 5 to 6 membered heteroaryl. In embodiments, R³ is substituted or unsubstituted pyrazinyl, pyrimidinyl, furanyl, thienyl, pyrrolyl, imidazolyl, pyrazolyl, oxazolyl, isoxazolyl, or thiazolyl.

In embodiments, R³ is substituted or unsubstituted phenyl. In embodiments, R³ is substituted or unsubstituted pyridyl. In embodiments, R³ is substituted or unsubstituted cyclohexyl. In embodiments, R³ is substituted or unsubstituted morpholinyl. In embodiments, R³ is substituted or unsubstituted piperazinyl. In embodiments, R³ is substituted or unsubstituted furanyl. In embodiments, R³ is substituted or unsubstituted thiazolyl. In embodiments, R³ is substituted or unsubstituted pyrazolyl. In embodiments, R³ is substituted or unsubstituted thienyl. In embodiments, R³ is substituted or unsubstituted pyrazinyl. In embodiments, R³ is substituted or unsubstituted pyrimidinyl. In embodiments, R³ is substituted or unsubstituted pyridazinyl. In embodiments, R³ is substituted or unsubstituted triazinyl. In embodiments, R³ is substituted or unsubstituted tetrazinyl. In embodiments, R³ is substituted or unsubstituted tetrazolyl. In embodiments, R³ is substituted or unsubstituted triazolyl. In embodiments, R³ is substituted or unsubstituted quinolinyl. In embodiments, R³ is substituted or unsubstituted isoquinolinyl. In embodiments, R³ is substituted or unsubstituted quinazolinyl. In embodiments, R³ is substituted or unsubstituted quinoxalinyl. In embodiments, R³ is substituted or unsubstituted imidazolyl. In embodiments, R³ is substituted or unsubstituted oxazolyl. In embodiments, R³ is substituted or unsubstituted isoxazolyl. In embodiments, R³ is substituted or unsubstituted thiazolyl. In embodiments, R³ is substituted or unsubstituted piperidinyl. In embodiments, R³ is substituted or unsubstituted thiomorpholinyl. In embodiments, R³ is substituted or unsubstituted thianyl. In embodiments, R³ is substituted or unsubstituted oxanyl. In embodiments, R³ is substituted or unsubstituted tetrahydropuranyl. In embodiments, R³ is substituted or unsubstituted dihydropuranyl. In embodiments, R³ is substituted or unsubstituted dioxanyl. In embodiments, R³ is substituted or unsubstituted pyrazolyl. In embodiments, R³ is substituted or unsubstituted pyrrolyl. In embodiments, R³ is substituted or unsubstituted thienyl. In embodiments, R³ is substituted or unsubstituted benzofuranyl. In embodiments, R³ is substituted or unsubstituted indolyl. In embodiments, R³ is substituted or unsubstituted benzothienyl. In embodiments, R³ is substituted or unsubstituted benzimidazolyl. In embodiments, R³ is substituted or unsubstituted isobenzofuranyl. In embodiments, R³ is substituted or unsubstituted isoindolyl. In embodiments, R³ is substituted or unsubstituted benzo[c]thienyl. In embodiments, R³ is substituted or unsubstituted purinyl. In embodiments, R³ is substituted or unsubstituted indazolyl. In embodiments, R³ is substituted or unsubstituted benzoxazolyl. In embodiments, R³ is substituted or unsubstituted benzisoxazolyl. In embodiments, R³ is substituted or unsubstituted benzothiazolyl. In embodiments, R³ is substituted or unsubstituted cyclopentyl. In embodiments, R³ is substituted or unsubstituted cyclobutyl. In embodiments, R³ is substituted or unsubstituted 2-thienyl. In embodiments, R³ is substituted or unsubstituted 3-thienyl. In embodiments, R³ is substituted or unsubstituted 2-furanyl. In embodiments, R³ is substituted or unsubstituted 3-furanyl. In embodiments, R³ is substituted or unsubstituted 2-pyridyl. In embodiments, R³ is substituted or unsubstituted 3-pyridyl. In embodiments, R³ is substituted or unsubstituted 4-pyridyl. In embodiments, R³ is substituted or unsubstituted 3-pyrazolyl. In embodiments, R³ is substituted or unsubstituted 4-pyrazolyl. In embodiments, R³ is substituted or unsubstituted 5-pyrazolyl. In embodiments, R³ is substituted or unsubstituted 2-pyrrolyl. In embodiments, R³ is substituted or unsubstituted 3-pyrrolyl. In embodiments, R³ is substituted or unsubstituted 2-thiazolyl. In embodiments, R³ is substituted or unsubstituted 4-thiazolyl. In embodiments, R³ is substituted or unsubstituted 5-thiazolyl. In embodiments, R³ is substituted or unsubstituted 2-pyridyl. In embodiments, R³ is substituted or unsubstituted 3-pyridyl. In embodiments, R³ is substituted or unsubstituted 4-pyridyl. In embodiments, R³ is substituted or unsubstituted phenyl.

In embodiments, R³ is an unsubstituted cycloalkyl. In embodiments, R³ is an unsubstituted C₃-C₈ cycloalkyl. In embodiments, R³ is an unsubstituted C₃-C₇ cycloalkyl. In embodiments, R³ is an unsubstituted C₃-C₆ cycloalkyl. In embodiments, R³ is an unsubstituted C₃-C₅ cycloalkyl. In embodiments, R³ is an unsubstituted C₃-C₄ cycloalkyl. In embodiments, R³ is an unsubstituted C₄-C₈ cycloalkyl. In embodiments, R³ is an unsubstituted C₅-C₈ cycloalkyl. In embodiments, R³ is an unsubstituted C₆-C₈ cycloalkyl. In embodiments, R³ is an unsubstituted C₅-C₆ cycloalkyl. In embodiments, R³ is an unsubstituted C₃ cycloalkyl. In embodiments, R³ is an unsubstituted C₄ cycloalkyl. In embodiments, R³ is an unsubstituted C₅ cycloalkyl. In embodiments, R³ is an unsubstituted C₆ cycloalkyl. In embodiments, R³ is an unsubstituted C₇ cycloalkyl. In embodiments, R³ is an unsubstituted C₈ cycloalkyl. In embodiments, R³ is an unsubstituted cyclopropyl. In embodiments, R³ is an unsubstituted cyclobutyl. In embodiments, R³ is an unsubstituted cyclopentyl. In embodiments, R³ is an unsubstituted cyclohexyl. In embodiments, R³ is an unsubstituted cycloheptyl. In embodiments, R³ is an unsubstituted a heterocycloalkyl. In embodiments, R³ is an unsubstituted 3 to 8 membered heterocycloalkyl. In embodiments, R³ is an unsubstituted 3 to 7 membered heterocycloalkyl. In embodiments, R³ is an unsubstituted 3 to 6 membered heterocycloalkyl. In embodiments, R³ is an unsubstituted 3 to 5 membered heterocycloalkyl. In embodiments, R³ is an unsubstituted 3 to 4 membered heterocycloalkyl. In embodiments, R³ is an unsubstituted 4 to 8 membered heterocycloalkyl. In embodiments, R³ is an unsubstituted 5 to 8 membered heterocycloalkyl. In embodiments, R³ is an unsubstituted 6 to 8 membered heterocycloalkyl. In embodiments, R³ is an unsubstituted 5 to 6 membered heterocycloalkyl. In embodiments, R³ is an unsubstituted 3 membered heterocycloalkyl. In embodiments, R³ is an unsubstituted 4 membered heterocycloalkyl. In embodiments, R³ is an unsubstituted 5 membered heterocycloalkyl. In embodiments, R³ is an unsubstituted 6 membered heterocycloalkyl. In embodiments, R³ is an unsubstituted 7 membered heterocycloalkyl. In embodiments, R³ is an unsubstituted 8 membered heterocycloalkyl. In embodiments, R³ is an unsubstituted aryl or unsubstituted heteroaryl. In embodiments, R³ is an unsubstituted aryl. In embodiments, R³ is an unsubstituted heteroaryl. In embodiments, R³ is an unsubstituted C₆-C₁₀ aryl. In embodiments, R³ is an unsubstituted C₁₀ aryl. In embodiments, R³ is an unsubstituted phenyl. In embodiments, R³ is an unsubstituted 5 to 10 membered heteroaryl. In embodiments, R³ is an unsubstituted 5 to 9 membered heteroaryl. In embodiments, R³ is an unsubstituted 5 to 6 membered heteroaryl. In embodiments, R³ is an unsubstituted 10 membered heteroaryl. In embodiments, R³ is an unsubstituted 9 membered heteroaryl. In embodiments, R³ is an unsubstituted 5 membered heteroaryl. In embodiments, R³ is an unsubstituted 6 membered heteroaryl. In embodiments, R³ is an unsubstituted phenyl. In embodiments, R³ is an unsubstituted pyridyl. In embodiments, R³ is an unsubstituted pyrazolyl. In embodiments, R³ is an unsubstituted imidazolyl. In embodiments, R³ is an unsubstituted oxazolyl. In embodiments, R³ is an unsubstituted isoxazolyl. In embodiments, R³ is an unsubstituted thiazolyl. In embodiments, R³ is an unsubstituted furanyl. In embodiments, R³ is an unsubstituted pyrrolyl. In embodiments, R³ is an unsubstituted thienyl. In embodiments, R³ is an unsubstituted two fused ring aryl. In embodiments, R³ is an unsubstituted two fused ring heteroaryl.

In embodiments, R³ is an unsubstituted aryl or unsubstituted heteroaryl. In embodiments, R³ is an unsubstituted phenyl or unsubstituted 5 to 6 membered heteroaryl. In embodiments, R³ is an unsubstituted phenyl. In embodiments, R³ is an unsubstituted 5 to 6 membered heteroaryl. In embodiments, R³ is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

In embodiments, R³ is an unsubstituted phenyl. In embodiments, R³ is an unsubstituted pyridyl. In embodiments, R³ is an unsubstituted cyclohexyl. In embodiments, R³ is an unsubstituted morpholinyl. In embodiments, R³ is an unsubstituted piperazinyl. In embodiments, R³ is an unsubstituted furanyl. In embodiments, R³ is an unsubstituted thiazolyl. In embodiments, R³ is an unsubstituted pyrazolyl. In embodiments, R³ is an unsubstituted thienyl. In embodiments, R³ is an unsubstituted pyrazinyl. In embodiments, R³ is an unsubstituted pyrimidinyl. In embodiments, R³ is an unsubstituted pyridazinyl. In embodiments, R³ is an unsubstituted triazinyl. In embodiments, R³ is an unsubstituted tetrazinyl. In embodiments, R³ is an unsubstituted tetrazolyl. In embodiments, R³ is an unsubstituted triazolyl. In embodiments, R³ is an unsubstituted quinolinyl. In embodiments, R³ is an unsubstituted isoquinolinyl. In embodiments, R³ is an unsubstituted quinazolinyl. In embodiments, R³ is an unsubstituted quinoxalinyl. In embodiments, R³ is an unsubstituted imidazolyl. In embodiments, R³ is an unsubstituted oxazolyl. In embodiments, R³ is an unsubstituted isoxazolyl. In embodiments, R³ is an unsubstituted thiazolyl. In embodiments, R³ is an unsubstituted piperidinyl. In embodiments, R³ is an unsubstituted thiomorpholinyl. In embodiments, R³ is an unsubstituted thianyl. In embodiments, R³ is an unsubstituted oxanyl. In embodiments, R³ is an unsubstituted tetrahydropuranyl. In embodiments, R³ is an unsubstituted dihydropuranyl. In embodiments, R³ is an unsubstituted dioxanyl. In embodiments, R³ is an unsubstituted pyrazolyl. In embodiments, R³ is an unsubstituted pyrrolyl. In embodiments, R³ is an unsubstituted thienyl. In embodiments, R³ is an unsubstituted benzofuranyl. In embodiments, R³ is an unsubstituted indolyl. In embodiments, R³ is an unsubstituted benzothienyl. In embodiments, R³ is an unsubstituted benzimidazolyl. In embodiments, R³ is an unsubstituted isobenzofuranyl. In embodiments, R³ is an unsubstituted isoindolyl. In embodiments, R³ is an unsubstituted benzo[c]thienyl. In embodiments, R³ is an unsubstituted purinyl. In embodiments, R³ is an unsubstituted indazolyl. In embodiments, R³ is an unsubstituted benzoxazolyl. In embodiments, R³ is an unsubstituted benzisoxazolyl. In embodiments, R³ is an unsubstituted benzothiazolyl. In embodiments, R³ is an unsubstituted cyclopentyl. In embodiments, R³ is an unsubstituted cyclobutyl. In embodiments, R³ is an unsubstituted 2-thienyl. In embodiments, R³ is an unsubstituted 3-thienyl. In embodiments, R³ is an unsubstituted 2-furanyl. In embodiments, R³ is an unsubstituted 3-furanyl. In embodiments, R³ is an unsubstituted 2-pyridyl. In embodiments, R³ is an unsubstituted 3-pyridyl. In embodiments, R³ is an unsubstituted 4-pyridyl. In embodiments, R³ is an unsubstituted 3-pyrazolyl. In embodiments, R³ is an unsubstituted 4-pyrazolyl. In embodiments, R³ is an unsubstituted 5-pyrazolyl. In embodiments, R³ is an unsubstituted 2-pyrrolyl. In embodiments, R³ is an unsubstituted 3-pyrrolyl. In embodiments, R³ is an unsubstituted 2-thiazolyl. In embodiments, R³ is an unsubstituted 4-thiazolyl. In embodiments, R³ is an unsubstituted 5-thiazolyl.

In embodiments, R^{3A} is independently hydrogen. In embodiments, R^{3A} is independently —CX^{3A}₃. In embodiments, R^{3A} is independently —CHX^{3A}₂. In embodiments, R^{3A} is independently —CH₂X^{3A}. In embodiments, R^{3A} is independently —CN. In embodiments, R^{3A} is independently —COOH. In embodiments, R^{3A} is independently —CONH₂.

In embodiments, R^{3A} is independently substituted or unsubstituted alkyl (e.g., C₁-C₈, C₁-C₆, or C₁-C₄). In embodiments, R^{3A} is independently substituted alkyl (e.g., C₁-C₈, C₁-C₆, or C₁-C₄). In embodiments, R^{3A} is independently unsubstituted alkyl (e.g., C₁-C₈, C₁-C₆, or C₁-C₄). In embodiments, R^{3A} is independently substituted or unsubstituted heteroalkyl (e.g., 2 to 8 membered, 2 to 6 membered, or 2 to 4 membered). In embodiments, R^{3A} is independently substituted heteroalkyl (e.g., 2 to 8 membered, 2 to 6 membered, or 2 to 4 membered). In embodiments, R^{3A} is independently unsubstituted heteroalkyl (e.g., 2 to 8 membered, 2 to 6 membered, or 2 to 4 membered). In embodiments, R^{3A} is independently substituted or unsubstituted cycloalkyl (e.g., C₃-C₈, C₃-C₆, or C₅-C₆). In embodiments, R^{3A} is independently substituted cycloalkyl (e.g., C₃-C₈, C₃-C₆, or C₅-C₆). In embodiments, R^{3A} is independently unsubstituted cycloalkyl (e.g., C₃-C₈, C₃-C₆, or C₅-C₆). In embodiments, R^{3A} is independently substituted or unsubstituted heterocycloalkyl (e.g., 3 to 8 membered, 3 to 6 membered, or 5 to 6 membered). In embodiments, R^{3A} is independently substituted heterocycloalkyl (e.g., 3 to 8 membered, 3 to 6 membered, or 5 to 6 membered). In embodiments, R^{3A} is independently unsubstituted heterocycloalkyl (e.g., 3 to 8 membered, 3 to 6 membered, or 5 to 6 membered). In embodiments, R^{3A} is independently substituted or unsubstituted aryl (e.g., C₆-C₁₀, C₁₀, or phenyl). In embodiments, R^{3A} is independently substituted aryl (e.g., C₆-C₁₀, C₁₀, or phenyl). In embodiments, R^{3A} is independently unsubstituted aryl (e.g., C₆-C₁₀, C₁₀, or phenyl). In embodiments, R^{3A} is independently substituted or unsubstituted heteroaryl (e.g., 5 to 10 membered, 5 to 9 membered, or 5 to 6 membered). In embodiments, R^{3A} is independently substituted heteroaryl (e.g., 5 to 10 membered, 5 to 9 membered, or 5 to 6 membered). In embodiments, R^{3A} is independently unsubstituted heteroaryl (e.g., 5 to 10 membered, 5 to 9 membered, or 5 to 6 membered). In embodiments, R^{3A} is independently unsubstituted methyl. In embodiments, R^{3A} is independently unsubstituted ethyl. In embodiments, R^{3A} is independently unsubstituted propyl. In embodiments, R^{3A} is independently unsubstituted isopropyl. In embodiments, R^{3A} is independently unsubstituted tert-butyl.

In embodiments, R^{3B} is independently hydrogen. In embodiments, R^{3B} is independently —CX^{3B}₃. In embodiments, R^{3B} is independently —CHX^{3B}₂. In embodiments, R^{3B} is independently —CH₂X^{3B}. In embodiments, R^{3B} is independently —CN. In embodiments, R^{3B} is independently —COOH. In embodiments, R^{3B} is independently —CONH₂.

In embodiments, R^{3B} is independently substituted or unsubstituted alkyl (e.g., C₁-C₈, C₁-C₆, or C₁-C₄). In embodiments, R^{3B} is independently substituted alkyl (e.g., C₁-C₈, C₁-C₆, or C₁-C₄). In embodiments, R^{3B} is independently unsubstituted alkyl (e.g., C₁-C₈, C₁-C₆, or C₁-C₄). In embodiments, R^{3B} is independently substituted or unsubstituted heteroalkyl (e.g., 2 to 8 membered, 2 to 6 membered, or 2 to 4 membered). In embodiments, R^{3B} is independently substituted heteroalkyl (e.g., 2 to 8 membered, 2 to 6 membered, or 2 to 4 membered). In embodiments, R^{3B} is independently unsubstituted heteroalkyl (e.g., 2 to 8 membered, 2 to 6 membered, or 2 to 4 membered). In embodiments, R^{3B} is independently substituted or unsubstituted cycloalkyl (e.g., C₃-C₈, C₃-C₆, or C₅-C₆). In embodiments, R^{3B} is independently substituted cycloalkyl (e.g., C₃-C₈, C₃-C₆, or C₅-C₆). In embodiments, R^{3B} is independently unsubstituted cycloalkyl (e.g., C₃-C₈, C₃-C₆, or C₅-C₆). In embodiments, R^{3B} is independently substituted or unsubstituted heterocycloalkyl (e.g., 3 to 8 membered, 3 to 6 membered, or 5 to 6 membered). In embodiments, R^{3B} is independently substituted heterocycloalkyl (e.g., 3 to 8 membered, 3 to 6 membered, or 5 to 6 membered). In embodiments, R^{3B} is independently unsubstituted heterocycloalkyl (e.g., 3 to 8 membered, 3 to 6 membered, or 5 to 6 membered). In embodiments, R^{3B} is independently substituted or unsubstituted aryl (e.g., C₆-C₁₀, C₁₀, or phenyl). In embodiments, R^{3B} is independently substituted aryl (e.g., C₆-C₁₀, C₁₀, or phenyl). In embodiments, R^{3B} is independently unsubstituted aryl (e.g., C₆-C₁₀, C₁₀, or phenyl). In embodiments, R^{3B} is independently substituted or unsubstituted heteroaryl (e.g., 5 to 10 membered, 5 to 9 membered, or 5 to 6 membered). In embodiments, R^{3B} is independently substituted heteroaryl (e.g., 5 to 10 membered, 5 to 9 membered, or 5 to 6 membered). In embodiments, R^{3B} is independently unsubstituted heteroaryl (e.g., 5 to 10 membered, 5 to 9 membered, or 5 to 6 membered). In embodiments, R^{3B} is independently unsubstituted methyl. In embodiments, R^{3B} is independently unsubstituted ethyl. In embodiments, R^{3B} is independently unsubstituted propyl. In embodiments, R^{3B} is independently unsubstituted isopropyl. In embodiments, R^{3B} is independently unsubstituted tert-butyl.

In embodiments, R^{3A} and R^{3B} substituents bonded to the same nitrogen atom may be joined to form a substituted or unsubstituted heterocycloalkyl. In embodiments, R^{3A} and R^{3B} substituents bonded to the same nitrogen atom may be joined to form a substituted or unsubstituted heteroaryl. In embodiments, R^{3A} and R^{3B} substituents bonded to the same nitrogen atom may be joined to form a substituted heterocycloalkyl. In embodiments, R^{3A} and R^{3B} substituents bonded to the same nitrogen atom may be joined to form a substituted heteroaryl. In embodiments, R^{3A} and R^{3B} substituents bonded to the same nitrogen atom may be joined to form an unsubstituted heterocycloalkyl. In embodiments, R^{3A} and R^{3B} substituents bonded to the same nitrogen atom may be joined to form an unsubstituted heteroaryl. In embodiments, R^{3A} and R^{3B} substituents bonded to the same nitrogen atom may be joined to form a substituted or unsubstituted 3 to 8 membered heterocycloalkyl. In embodiments, R^{3A} and R^{3B} substituents bonded to the same nitrogen atom may be joined to form a substituted or unsubstituted 5 to 10 membered heteroaryl. In embodiments, R^{3A} and R^{3B} substituents bonded to the same nitrogen atom may be joined to form a substituted 3 to 8 membered heterocycloalkyl. In embodiments, R^{3A} and R^{3B} substituents bonded to the same nitrogen atom may be joined to form a substituted 5 to 10 membered heteroaryl. In embodiments, R^{3A} and R^{3B} substituents bonded to the same nitrogen atom may be joined to form an unsubstituted 3 to 8 membered heterocycloalkyl. In embodiments, R^{3A} and R^{3B} substituents bonded to the same nitrogen atom may be joined to form an unsubstituted 5 to 10 membered heteroaryl. In embodiments, R^{3A} and R^{3B} substituents bonded to the same nitrogen atom may be joined to form a substituted or unsubstituted 3 to 6 membered heterocycloalkyl. In embodiments, R^{3A} and R^{3B} substituents bonded to the same nitrogen atom may be joined to form a substituted or unsubstituted 5 to 6 membered heteroaryl. In embodiments, R^{3A} and R^{3B} substituents bonded to the same nitrogen atom may be joined to form a substituted 3 to 6 membered heterocycloalkyl. In embodiments, R^{3A} and R^{3B} substituents bonded to the same nitrogen atom may be joined to form a substituted 5 to 6 membered heteroaryl. In embodiments, R^{3A} and R^{3B} substituents bonded to the same nitrogen atom may be joined to form an unsubstituted 3 to 6 membered heterocycloalkyl. In embodiments, R^{3A} and R^{3B} substituents bonded to the same nitrogen atom may be joined to form an unsubstituted 5 to 6 membered heteroaryl.

In embodiments, R^{3C} is independently hydrogen. In embodiments, R³ is independently —CX^{3C}₃. In embodiments, R^{3C} is independently —CHX^{3C}₂. In embodiments, R^{3C} is independently —CH₂X^{3C}. In embodiments, R^{3C} is independently —CN. In embodiments, R^{3C} is independently —COOH. In embodiments, R^{3C} is independently —CONH₂.

In embodiments, R^{3C} is independently substituted or unsubstituted alkyl (e.g., C₁-C₈, C₁-C₆, or C₁-C₄). In embodiments, R^{3C} is independently substituted alkyl (e.g., C₁-C₈, C₁-C₆, or C₁-C₄). In embodiments, R^{3C} is independently unsubstituted alkyl (e.g., C₁-C₈, C₁-C₆, or C₁-C₄). In embodiments, R^{3C} is independently substituted or unsubstituted heteroalkyl (e.g., 2 to 8 membered, 2 to 6 membered, or 2 to 4 membered). In embodiments, R^{3C} is independently substituted heteroalkyl (e.g., 2 to 8 membered, 2 to 6 membered, or 2 to 4 membered). In embodiments, R^{3C} is independently unsubstituted heteroalkyl (e.g., 2 to 8 membered, 2 to 6 membered, or 2 to 4 membered). In embodiments, R^{3C} is independently substituted or unsubstituted cycloalkyl (e.g., C₃-C₈, C₃-C₆, or C₅-C₆). In embodiments, R^{3C} is independently substituted cycloalkyl (e.g., C₃-C₈, C₃-C₆, or C₅-C₆). In embodiments, R^{3C} is independently unsubstituted cycloalkyl (e.g., C₃-C₈, C₃-C₆, or C₅-C₆). In embodiments, R^{3C} is independently substituted or unsubstituted heterocycloalkyl (e.g., 3 to 8 membered, 3 to 6 membered, or 5 to 6 membered). In embodiments, R^{3C} is independently substituted heterocycloalkyl (e.g., 3 to 8 membered, 3 to 6 membered, or 5 to 6 membered). In embodiments, R^{3C} is independently unsubstituted heterocycloalkyl (e.g., 3 to 8 membered, 3 to 6 membered, or 5 to 6 membered). In embodiments, R^{3C} is independently substituted or unsubstituted aryl (e.g., C₆-C₁₀, C₁₀, or phenyl). In embodiments, R^{3C} is independently substituted aryl (e.g., C₆-C₁₀, C₁₀, or phenyl). In embodiments, R^{3C} is independently unsubstituted aryl (e.g., C₆-C₁₀, C₁₀, or phenyl). In embodiments, R^{3C} is independently substituted or unsubstituted heteroaryl (e.g., 5 to 10 membered, 5 to 9 membered, or 5 to 6 membered). In embodiments, R^{3C} is independently substituted heteroaryl (e.g., 5 to 10 membered, 5 to 9 membered, or 5 to 6 membered). In embodiments, R^{3C} is independently unsubstituted heteroaryl (e.g., 5 to 10 membered, 5 to 9 membered, or 5 to 6 membered). In embodiments, R^{3C} is independently unsubstituted methyl. In embodiments, R^{3C} is independently unsubstituted ethyl. In embodiments, R³ is independently unsubstituted propyl. In embodiments, R^{3C} is independently unsubstituted isopropyl. In embodiments, R^{3C} is independently unsubstituted tert-butyl.

In embodiments, R^{3D} is independently hydrogen. In embodiments, R^{3D} is independently —CX^{3D}₃. In embodiments, R^{3D} is independently —CHX^{3D}₂. In embodiments, R^{3D} is independently —CH₂X^{3D}. In embodiments, R^{3D} is independently —CN. In embodiments, R^{3D} is independently —COOH. In embodiments, R^{3D} is independently —CONH₂. In embodiments, R^{3D} is independently —CF₃. In embodiments, R^{3D} is independently —CHF₂. In embodiments, R^{3D} is independently —CH₂F. In embodiments, R^{3D} is independently —CCl₃. In embodiments, R^{3D} is independently —CHCl₂. In embodiments, R^{3D} is independently —CH₂Cl. In embodiments, R^{3D} is independently —CBr₃. In embodiments, R^{3D} is independently —CHBr₂. In embodiments, R^{3D} is independently —CH₂Br. In embodiments, R^{3D} is independently —CI₃. In embodiments, R^{3D} is independently —CHI₂. In embodiments, R^{3D} is independently —CH₂I.

In embodiments, R^{3D} is independently substituted or unsubstituted alkyl (e.g., C₁-C₈, C₁-C₆, or C₁-C₄). In embodiments, R^{3D} is independently substituted alkyl (e.g., C₁-C₈, C₁-C₆, or C₁-C₄). In embodiments, R^{3D} is independently unsubstituted alkyl (e.g., C₁-C₈, C₁-C₆, or C₁-C₄). In embodiments, R^{3D} is independently substituted or unsubstituted heteroalkyl (e.g., 2 to 8 membered, 2 to 6 membered, or 2 to 4 membered). In embodiments, R^{3D} is independently substituted heteroalkyl (e.g., 2 to 8 membered, 2 to 6 membered, or 2 to 4 membered). In embodiments, R^{3D} is independently unsubstituted heteroalkyl (e.g., 2 to 8 membered, 2 to 6 membered, or 2 to 4 membered). In embodiments, R^{3D} is independently substituted or unsubstituted cycloalkyl (e.g., C₃-C₈, C₃-C₆, or C₅-C₆). In embodiments, R^{3D} is independently substituted cycloalkyl (e.g., C₃-C₈, C₃-C₆, or C₅-C₆). In embodiments, R^{3D} is independently unsubstituted cycloalkyl (e.g., C₃-C₈, C₃-C₆, or C₅-C₆). In embodiments, R^{3D} is independently substituted or unsubstituted heterocycloalkyl (e.g., 3 to 8 membered, 3 to 6 membered, or 5 to 6 membered). In embodiments, R^{3D} is independently substituted heterocycloalkyl (e.g., 3 to 8 membered, 3 to 6 membered, or 5 to 6 membered). In embodiments, R^{3D} is independently unsubstituted heterocycloalkyl (e.g., 3 to 8 membered, 3 to 6 membered, or 5 to 6 membered). In embodiments, R^{3D} is independently substituted or unsubstituted aryl (e.g., C₆-C₁₀, C₁₀, or phenyl). In embodiments, R^{3D} is independently substituted aryl (e.g., C₆-C₁₀, C₁₀, or phenyl). In embodiments, R^{3D} is independently unsubstituted aryl (e.g., C₆-C₁₀, C₁₀, or phenyl). In embodiments, R^{3D} is independently substituted or unsubstituted heteroaryl (e.g., 5 to 10 membered, 5 to 9 membered, or 5 to 6 membered). In embodiments, R^{3D} is independently substituted heteroaryl (e.g., 5 to 10 membered, 5 to 9 membered, or 5 to 6 membered). In embodiments, R^{3D} is independently unsubstituted heteroaryl (e.g., 5 to 10 membered, 5 to 9 membered, or 5 to 6 membered). In embodiments, R^{3D} is independently unsubstituted methyl. In embodiments, R^{3D} is independently unsubstituted ethyl. In embodiments, R^{3D} is independently unsubstituted propyl. In embodiments, R^{3D} is independently unsubstituted isopropyl. In embodiments, R^{3D} is independently unsubstituted tert-butyl.

In embodiments, R³ is independently hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, R²⁶-substituted or unsubstituted alkyl, R²⁶-substituted or unsubstituted heteroalkyl, R²⁶-substituted or unsubstituted cycloalkyl, R²⁶-substituted or unsubstituted heterocycloalkyl, R²⁶-substituted or unsubstituted aryl, or R²⁶-substituted or unsubstituted heteroaryl.

In embodiments, R³ is independently halogen, —CX³₃, —CHX³₂, —CH₂X³, —OCX³₃, —OCH₂X³, —OCHX³₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²⁶-substituted or unsubstituted alkyl, R²⁶-substituted or unsubstituted heteroalkyl, R²⁶-substituted or unsubstituted cycloalkyl, R²⁶-substituted or unsubstituted heterocycloalkyl, R²⁶-substituted or unsubstituted aryl, or R²⁶-substituted or unsubstituted heteroaryl.

In embodiments, R³ is independently halogen, —CX³₃, —CHX³₂, —CH₂X³, —OCX³₃, —OCH₂X³, —OCHX³₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²⁶-substituted or unsubstituted C₁-C₈ alkyl, R²⁶-substituted or unsubstituted 2 to 8 membered heteroalkyl, R²⁶-substituted or unsubstituted C₃-C₈ cycloalkyl, R²⁶-substituted or unsubstituted 3 to 6 membered heterocycloalkyl, R²⁶-substituted or unsubstituted phenyl, or R²⁶-substituted or unsubstituted 5 to 6 membered heteroaryl. X³ is —F, —Cl, —Br, or —I. In embodiments, R³ is independently hydrogen. In embodiments, R³ is independently methyl. In embodiments, R³ is independently ethyl. In embodiments, R³ is independently —OCH₃. In embodiments, R³ is independently —OCH₂CH₃. In embodiments, R³ is independently —OCF₃. In embodiments, R³ is independently —OCHF₂. In embodiments, R³ is independently —OCH₂F. In embodiments, R³ is independently —OCBr₃. In embodiments, R³ is independently —OCHBr₂. In embodiments, R³ is independently —OCH₂Br. In embodiments, R³ is independently —OCCl₃. In embodiments, R³ is independently —OCHCl₂. In embodiments, R³ is independently —OCH₂Cl. In embodiments, R³ is independently —OCl₃. In embodiments, R³ is independently —OCHI₂. In embodiments, R³ is independently —OCH₂I. In embodiments, R³ is independently —OCH₂CF₃. In embodiments, R³ is independently —OCH₂CX³₃.

R²⁶ is independently oxo, halogen, —CX²⁶₃, —CHX²⁶₂, —CH₂X²⁶, —OCX²⁶₃, —OCH₂X²⁶, —OCHX²⁶₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²⁷-substituted or unsubstituted alkyl (e.g., C₁-C₈, C₁-C₆, or C₁-C₄), R²⁷-substituted or unsubstituted heteroalkyl (e.g., 2 to 8 membered, 2 to 6 membered, or 2 to 4 membered), R²⁷-substituted or unsubstituted cycloalkyl (e.g., C₃-C₈, C₃-C₆, or C₅-C₆), R²⁷-substituted or unsubstituted heterocycloalkyl (e.g., 3 to 8 membered, 3 to 6 membered, or 5 to 6 membered), R²⁷-substituted or unsubstituted aryl (e.g., C₆-C₁₀, C₁₀, or phenyl), or R²⁷-substituted or unsubstituted heteroaryl (e.g., 5 to 10 membered, 5 to 9 membered, or 5 to 6 membered). X²⁶ is —F, —Cl, —Br, or —I.

R²⁷ is independently oxo, halogen, —CX²⁷₃, —CHX²⁷₂, —CH₂X²⁷, —OCX²⁷₃, —OCH₂X²⁷, —OCHX²⁷₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —R²⁸-substituted or unsubstituted alkyl (e.g., C₁-C₈, C₁-C₆, or C₁-C₄), R²⁸-substituted or unsubstituted heteroalkyl (e.g., 2 to 8 membered, 2 to 6 membered, or 2 to 4 membered), R²⁸-substituted or unsubstituted cycloalkyl (e.g., C₃-C₈, C₃-C₆, or C₅-C₆), R²⁸-substituted or unsubstituted heterocycloalkyl (e.g., 3 to 8 membered, 3 to 6 membered, or 5 to 6 membered), R²⁸-substituted or unsubstituted aryl (e.g., C₆-C₁₀, C₁₀, or phenyl), or R²⁸-substituted or unsubstituted heteroaryl (e.g., 5 to 10 membered, 5 to 9 membered, or 5 to 6 membered). X²⁷ is —F, —Cl, —Br, or —I.

In embodiments, R^{3A} is independently hydrogen, —CX^{3A}₃, —CHX^{3A}₂, —CH₂X^{3A}, —OCX^{3A}₃, —OCH₂X^{3A}, —OCHX^{3A}₂, —CN, —COOH, —CONH₂, R^{26A}-substituted or unsubstituted alkyl, R^{26A}-substituted or unsubstituted heteroalkyl, R^{26A}-substituted or unsubstituted cycloalkyl, R^{26A}-substituted or unsubstituted heterocycloalkyl, R^{26A}-substituted or unsubstituted aryl, or R^{26A}-substituted or unsubstituted heteroaryl.

In embodiments, R^{3A} is independently hydrogen, —CX^{3A}₃, —CHX^{3A}₂, —CH₂X^{3A}, —CN, —COOH, —CONH₂, R^{26A}-substituted or unsubstituted C₁-C₈ alkyl, R^{26A}-substituted or unsubstituted 2 to 8 membered heteroalkyl, R^{26A}-substituted or unsubstituted C₃-C₈ cycloalkyl, R^{26A}-substituted or unsubstituted 3 to 6 membered heterocycloalkyl, R^{26A}-substituted or unsubstituted phenyl, or R^{26A}-substituted or unsubstituted 5 to 6 membered heteroaryl. X^{3A} is —F, —Cl, —Br, or —I. In embodiments, R^{3A} is independently hydrogen. In embodiments, R^{3A} is independently methyl. In embodiments, R^{3A} is independently ethyl.

In embodiments, R^{3A} and R^{3B} substituents bonded to the same nitrogen atom may optionally be joined to form a R^{26A}-substituted or unsubstituted heterocycloalkyl or R^{26A}-substituted or unsubstituted heteroaryl. In embodiments, R^{3A} and R^{3B} substituents bonded to the same nitrogen atom may optionally be joined to form a R^{26A}-substituted or unsubstituted 3 to 6 membered heterocycloalkyl or R^{26A}-substituted or unsubstituted 5 to 6 membered heteroaryl.

R^{26A} is independently oxo, halogen, —CX^{26A}₃, —CHX^{26A}₂, —CH₂X^{26A}, —OCX^{26A}₃, —OCH₂X^{26A}, —OCHX^{26A}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{27A}-substituted or unsubstituted alkyl (e.g., C₁-C₈, C₁-C₆, or C₁-C₄), R^{27A}-substituted or unsubstituted heteroalkyl (e.g., 2 to 8 membered, 2 to 6 membered, or 2 to 4 membered), R^{27A}-substituted or unsubstituted cycloalkyl (e.g., C₃-C₈, C₃-C₆, or C₅-C₆), R^{27A}-substituted or unsubstituted heterocycloalkyl (e.g., 3 to 8 membered, 3 to 6 membered, or 5 to 6 membered), R^{27A}-substituted or unsubstituted aryl (e.g., C₆-C₁₀, C₁₀, or phenyl), or R^{27A}-substituted or unsubstituted heteroaryl (e.g., 5 to 10 membered, 5 to 9 membered, or 5 to 6 membered). X^{26A} is —F, —Cl, —Br, or —I.

R^{27A} is independently oxo, halogen, —CX^{27A}₃, —CHX^{27A}₂, —CH₂X^{27A}, —OCX^{27A}₃, —OCH₂X^{27A}, —OCHX^{27A}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{28A}-substituted or unsubstituted alkyl (e.g., C₁-C₈, C₁-C₆, or C₁-C₄), R^{28A}-substituted or unsubstituted heteroalkyl (e.g., 2 to 8 membered, 2 to 6 membered, or 2 to 4 membered), R^{28A}-substituted or unsubstituted cycloalkyl (e.g., C₃-C₈, C₃-C₆, or C₅-C₆), R^{28A}-substituted or unsubstituted heterocycloalkyl (e.g., 3 to 8 membered, 3 to 6 membered, or 5 to 6 membered), R^{28A}-substituted or unsubstituted aryl (e.g., C₆-C₁₀, C₁₀, or phenyl), or R^{28A}-substituted or unsubstituted heteroaryl (e.g., 5 to 10 membered, 5 to 9 membered, or 5 to 6 membered). X^{27A} is —F, —Cl, —Br, or —I.

In embodiments, R^{3B} is independently hydrogen, —CX^{3A}₃, —CHX^{3B}₂, —CH₂X^{3B}, —CN, —COOH, —CONH₂, R^{26B}-substituted or unsubstituted alkyl, R^{26B}-substituted or unsubstituted heteroalkyl, R^{26B}-substituted or unsubstituted cycloalkyl, R^{26B}-substituted or unsubstituted heterocycloalkyl, R^{26B}-substituted or unsubstituted aryl, or R^{26B}-substituted or unsubstituted heteroaryl. In embodiments, R^{3B} is independently hydrogen, —CX^{3B}₃, —CN, —COOH, —CONH₂, —CHX^{3B}₂, —CH₂X^{3B}, R^{26B}-substituted or unsubstituted C₁-C₈ alkyl, R^{26B}-substituted or unsubstituted 2 to 8 membered heteroalkyl, R^{26B}-substituted or unsubstituted C₃-C₈ cycloalkyl, R^{26B}-substituted or unsubstituted 3 to 6 membered heterocycloalkyl, R^{26B}-substituted or unsubstituted phenyl, or R^{26B}-substituted or unsubstituted 5 to 6 membered heteroaryl. X^{3B} is —F, —Cl, —Br, or —I. In embodiments, R^{3B} is independently hydrogen. In embodiments, R^{3B} is independently methyl. In embodiments, R^{3B} is independently ethyl.

In embodiments, R^{3A} and R^{3B} substituents bonded to the same nitrogen atom may optionally be joined to form a R^{26B}-substituted or unsubstituted heterocycloalkyl or R^{26B}-substituted or unsubstituted heteroaryl. In embodiments, R^{3A} and R^{3B} substituents bonded to the same nitrogen atom may optionally be joined to form a R^{26B}-substituted or unsubstituted 3 to 6 membered heterocycloalkyl or R^{26B}-substituted or unsubstituted 5 to 6 membered heteroaryl.

R^{26B} is independently oxo, halogen, —CX^{26B}₃, —CHX^{26B}₂, —CH₂X^{26B}, —OCX^{26B}₃, —OCH₂X^{26B}, —OCHX^{26B}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{27B}-substituted or unsubstituted alkyl (e.g., C₁-C₈, C₁-C₆, or C₁-C₄), R^{27B}-substituted or unsubstituted heteroalkyl (e.g., 2 to 8 membered, 2 to 6 membered, or 2 to 4 membered), R^{27B}-substituted or unsubstituted cycloalkyl (e.g., C₃-C₈, C₃-C₆, or C₅-C₆), R^{27B}-substituted or unsubstituted heterocycloalkyl (e.g., 3 to 8 membered, 3 to 6 membered, or 5 to 6 membered), R^{27B}-substituted or unsubstituted aryl (e.g., C₆-C₁₀, C₁₀, or phenyl), or R^{27B}-substituted or unsubstituted heteroaryl (e.g., 5 to 10 membered, 5 to 9 membered, or 5 to 6 membered). X^{26B} is —F, —Cl, —Br, or —I.

R^{27B} is independently oxo, halogen, —CX^{27B}₃, —CHX^{27B}₂, —CH₂X^{27B}, —OCX^{27B}₃, —OCH₂X^{27B}, —OCHX^{27B}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{28B}-substituted or unsubstituted alkyl (e.g., C₁-C₈, C₁-C₆, or C₁-C₄), R^{28B}-substituted or unsubstituted heteroalkyl (e.g., 2 to 8 membered, 2 to 6 membered, or 2 to 4 membered), R^{28B}-substituted or unsubstituted cycloalkyl (e.g., C₃-C₈, C₃-C₆, or C₅-C₆), R^{28B}-substituted or unsubstituted heterocycloalkyl (e.g., 3 to 8 membered, 3 to 6 membered, or 5 to 6 membered), R^{28B}-substituted or unsubstituted aryl (e.g., C₆-C₁₀, C₁₀, or phenyl), or R^{28B}-substituted or unsubstituted heteroaryl (e.g., 5 to 10 membered, 5 to 9 membered, or 5 to 6 membered). X^{27B} is —F, —Cl, —Br, or —I.

In embodiments, R^{3C} is independently hydrogen, —CX^{3C}₃, —CN, —COOH, —CONH₂, —CHX^{3C}₂, —CH₂X^{3C}, R^{26C}-substituted or unsubstituted alkyl, R^{26C}-substituted or unsubstituted heteroalkyl, R^{26C}-substituted or unsubstituted cycloalkyl, R^{26C}-substituted or unsubstituted heterocycloalkyl, R^{26C}-substituted or unsubstituted aryl, or R^{26C}-substituted or unsubstituted heteroaryl. In embodiments, R^{3C} is independently hydrogen, —CX^{3C}₃, —CN, —COOH, —CONH₂, —CHX^{3C}₂, —CH₂X^{3C}, R^{26C}-substituted or unsubstituted C₁-C₈ alkyl, R^{26C}-substituted or unsubstituted 2 to 8 membered heteroalkyl, R^{26C}-substituted or unsubstituted C₃-C₈ cycloalkyl, R^{26C}-substituted or unsubstituted 3 to 6 membered heterocycloalkyl, R^{26C}-substituted or unsubstituted phenyl, or R^{26C}-substituted or unsubstituted 5 to 6 membered heteroaryl. X^{3C} is —F, —Cl, —Br, or —I. In embodiments, R^{3C} is independently hydrogen. In embodiments, R^{3C} is independently methyl. In embodiments, R^{3C} is independently ethyl.

R^{26C} is independently oxo, halogen, —CX^{26C}₃, —CHX^{26C}₂, —CH₂X^{26C}, —OCX^{26C}₃, —OCH₂X^{26C}, —OCHX^{26C}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{27C}-substituted or unsubstituted alkyl (e.g., C₁-C₈, C₁-C₆, or C₁-C₄), R^{27C}-substituted or unsubstituted heteroalkyl (e.g., 2 to 8 membered, 2 to 6 membered, or 2 to 4 membered), R^{27C}-substituted or unsubstituted cycloalkyl (e.g., C₃-C₈, C₃-C₆, or C₅-C₆), R^{27C}-substituted or unsubstituted heterocycloalkyl (e.g., 3 to 8 membered, 3 to 6 membered, or 5 to 6 membered), R^{27C}-substituted or unsubstituted aryl (e.g., C₆-C₁₀, C₁₀, or phenyl), or R^{27C}-substituted or unsubstituted heteroaryl (e.g., 5 to 10 membered, 5 to 9 membered, or 5 to 6 membered). X^{26C} is —F, —Cl, —Br, or —I.

R^{27C} is independently oxo, halogen, —CX^{27C}₃, —CHX^{27C}₂, —CH₂X^{27C}, —OCX^{27C}₃, —OCH₂X^{27C}, —OCHX^{27C}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{28C}-substituted or unsubstituted alkyl (e.g., C₁-C₈, C₁-C₆, or C₁-C₄), R^{28C}-substituted or unsubstituted heteroalkyl (e.g., 2 to 8 membered, 2 to 6 membered, or 2 to 4 membered), R^{28C}-substituted or unsubstituted cycloalkyl (e.g., C₃-C₈, C₃-C₆, or C₅-C₆), R^{28C}-substituted or unsubstituted heterocycloalkyl (e.g., 3 to 8 membered, 3 to 6 membered, or 5 to 6 membered), R^{28C}-substituted or unsubstituted aryl (e.g., C₆-C₁₀, C₁₀, or phenyl), or R^{28C}-substituted or unsubstituted heteroaryl (e.g., 5 to 10 membered, 5 to 9 membered, or 5 to 6 membered). X^{27C} is —F, —Cl, —Br, or —I.

In embodiments, R^{3D} is independently hydrogen, —CX^{3D}₃, —CN, —COOH, —CONH₂, —CHX^{3D}₂, —CH₂X^{3D}, R^{26D}-substituted or unsubstituted alkyl, R^{26D}-substituted or unsubstituted heteroalkyl, R^{26D}-substituted or unsubstituted cycloalkyl, R^{26D}-substituted or unsubstituted heterocycloalkyl, R^{26D}-substituted or unsubstituted aryl, or R^{26D}-substituted or unsubstituted heteroaryl. In embodiments, R^{3D} is independently hydrogen, —CX^{3D}₃, —CN, —COOH, —CONH₂, —CHX^{3D}₂, —CH₂X^{3D}, R^{26D}-substituted or unsubstituted C₁-C₈ alkyl, R^{26D}-substituted or unsubstituted 2 to 8 membered heteroalkyl, R^{26D}-substituted or unsubstituted C₃-C₈ cycloalkyl, R^{26D}-substituted or unsubstituted 3 to 6 membered heterocycloalkyl, R^{26D}-substituted or unsubstituted phenyl, or R^{26D}-substituted or unsubstituted 5 to 6 membered heteroaryl. X^{3D} is —F, —Cl, —Br, or —I. In embodiments, R^{3D} is independently hydrogen. In embodiments, R^{3D} is independently methyl. In embodiments, R^{3D} is independently ethyl.

R^{26D} is independently oxo, halogen, —CX^{26D}₃, —CHX^{26D}₂, —CH₂X^{26D}, —OCX^{26D}₃, —OCH₂X^{26D}, —OCHX^{26D}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{27D}-substituted or unsubstituted alkyl (e.g., C₁-C₈, C₁-C₆, or C₁-C₄), R^{27D}-substituted or unsubstituted heteroalkyl (e.g., 2 to 8 membered, 2 to 6 membered, or 2 to 4 membered), R^{27D}-substituted or unsubstituted cycloalkyl (e.g., C₃-C₈, C₃-C₆, or C₅-C₆), R^{27D}-substituted or unsubstituted heterocycloalkyl (e.g., 3 to 8 membered, 3 to 6 membered, or 5 to 6 membered), R^{27D}-substituted or unsubstituted aryl (e.g., C₆-C₁₀, C₁₀, or phenyl), or R^{27D}-substituted or unsubstituted heteroaryl (e.g., 5 to 10 membered, 5 to 9 membered, or 5 to 6 membered). X^{26D} is —F, —Cl, —Br, or —I.

R^{27D} is independently oxo, halogen, —CX^{27D}₃, —CHX^{27D}₂, —CH₂X^{27D}, —OCX^{27D}₃, —OCH₂X^{27D}, —OCHX^{27D}₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{28D}-substituted or unsubstituted alkyl (e.g., C₁-C₈, C₁-C₆, or C₁-C₄), R^{28D}-substituted or unsubstituted heteroalkyl (e.g., 2 to 8 membered, 2 to 6 membered, or 2 to 4 membered), R^{28D}-substituted or unsubstituted cycloalkyl (e.g., C₃-C₈, C₃-C₆, or C₅-C₆), R^{28D}-substituted or unsubstituted heterocycloalkyl (e.g., 3 to 8 membered, 3 to 6 membered, or 5 to 6 membered), R^{28D}-substituted or unsubstituted aryl (e.g., C₆-C₁₀, C₁₀, or phenyl), or R^{28D}-substituted or unsubstituted heteroaryl (e.g., 5 to 10 membered, 5 to 9 membered, or 5 to 6 membered). X^{27D} is —F, —Cl, —Br, or —I.

R²⁸, R^{28A}, R^{28B}, R^{28C}, and R^{28D} are independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, R⁴ is an unsubstituted methyl. In embodiments, R⁴ is —C(O)N(CH₃)₂. In embodiments, R⁴ is —CN. In embodiments, R⁴ is an unsubstituted methoxy. In embodiments, R⁴ is an unsubstituted tert-butyl. In embodiments, R⁴ is —OH. In embodiments, R⁴ is an unsubstituted ethoxy. In embodiments, R⁴ is —N(CH₃)₂. In embodiments, R⁴ is —SH. In embodiments, R⁴ is —SCH₃. In embodiments, R⁴ is —SCH₂CH₃. In embodiments, R⁴ is an unsubstituted ethyl. In embodiments, R⁴ is an unsubstituted propyl. In embodiments, R⁴ is an unsubstituted isopropyl. In embodiments, R⁴ is an unsubstituted butyl. In embodiments, R⁴ is an unsubstituted isobutyl. In embodiments, R⁴ is —NH₂. In embodiments, R⁴ is —C(CN). In embodiments, R⁴ is —NHCH₃. In embodiments, R⁴ is —NHCH₂CH₃. In embodiments, R⁴ is —N(CH₂CH₃)₂. In embodiments, R⁴ is —N(CH₃)(CH₂CH₃). In embodiments, R⁴ is —C(O)NH₂. In embodiments, R⁴ is —C(O)NHCH₃. In embodiments, R⁴ is —C(O)NHCH₂CH₃. In embodiments, R⁴ is —C(O)N(CH₂CH₃)₂. In embodiments, R⁴ is —C(O)N(CH₃)(CH₂CH₃). In embodiments, R⁴ is independently —CF₃. In embodiments, R⁴ is independently —CHF₂. In embodiments, R⁴ is independently —CH₂F. In embodiments, R⁴ is independently —CCl₃. In embodiments, R⁴ is independently —CHCl₂. In embodiments, R⁴ is independently —CH₂Cl. In embodiments, R⁴ is independently —CBr₃. In embodiments, R⁴ is independently —CHBr₂. In embodiments, R⁴ is independently —CH₂Br. In embodiments, R⁴ is independently —CI₃. In embodiments, R⁴ is independently —CHI₂. In embodiments, R⁴ is independently —CH₂I.

In embodiments, R⁴ is independently unsubstituted heteroalkyl. In embodiments, R⁴ is independently unsubstituted 2 to 5 membered heteroalkyl. In embodiments, R⁴ is independently —OCH₃, —OCH₂CH₃, —N(CH₃)₂, —NH₂, —NH(CH₃), —N(CH₂CH₃)₂, —NH(CH₂CH₃), or —SH. In embodiments, R⁴ is independently —OCH₃. In embodiments, R⁴ is independently —OCH₂CH₃. In embodiments, R⁴ is independently —N(CH₃)₂. In embodiments, R⁴ is independently —NH₂. In embodiments, R⁴ is independently —NH(CH₃). In embodiments, R⁴ is independently —N(CH₂CH₃)₂. In embodiments, R⁴ is independently —NH(CH₂CH₃). In embodiments, R⁴ is independently —SH. In embodiments, R⁴ is independently —OCH₂CH₂CH₃. In embodiments, R⁴ is independently unsubstituted methoxy. In embodiments, R⁴ is independently unsubstituted ethoxy. In embodiments, R⁴ is independently unsubstituted propoxy. In embodiments, R⁴ is independently unsubstituted isopropoxy. In embodiments, R⁴ is independently unsubstituted butoxy. In embodiments, R⁴ is independently unsubstituted tert-butoxy. In embodiments, R⁴ is independently unsubstituted pentoxy. In embodiments, R⁴ is independently unsubstituted hexoxy.

In embodiments, R⁴ is an unsubstituted methoxy. In embodiments, R⁴ is an unsubstituted tert-butyl. In embodiments, R⁴ is an unsubstituted phenoxy. In embodiments, R⁴ is an unsubstituted methyl. In embodiments, R⁴ is —OH. In embodiments, R⁴ is an unsubstituted ethoxy. In embodiments, R⁴ is —SH. In embodiments, R⁴ is —SCH₃. In embodiments, R⁴ is —SCH₂CH₃. In embodiments, R⁴ is an unsubstituted ethyl. In embodiments, R⁴ is an unsubstituted propyl. In embodiments, R⁴ is an unsubstituted isopropyl. In embodiments, R⁴ is an unsubstituted butyl. In embodiments, R⁴ is an unsubstituted isobutyl. In embodiments, R⁴ is —NH₂. In embodiments, R⁴ is —NHCH₃. In embodiments, R⁴ is —NHCH₂CH₃. In embodiments, R⁴ is —N(CH₂CH₃)₂. In embodiments, R⁴ is —N(CH₃)(CH₂CH₃). In embodiments, R⁴ is halogen. In embodiments, R⁴ is —F. In embodiments, R⁴ is —Cl. In embodiments, R⁴ is —I. In embodiments, R⁴ is —Br. In embodiments, R⁴ is independently —CF₃. In embodiments, R⁴ is independently —OCH₃. In embodiments, R⁴ is an unsubstituted phenyl. In embodiments, R⁴ is independently —C(O)N(CH₃)₂. In embodiments, R⁴ is independently —C(O)NH(CH₃). In embodiments, R⁴ is independently —C(O)N(CH₂CH₃)₂. In embodiments, R⁴ is independently —C(O)NH(CH₂CH₃). In embodiments, R⁴ is independently unsubstituted cyclohexyl. In embodiments, R⁴ is independently unsubstituted morpholinyl. In embodiments, R⁴ is independently unsubstituted piperazinyl. In embodiments, R⁴ is independently N-methyl substituted piperazinyl. In embodiments, R⁴ is independently unsubstituted pyridyl. In embodiments, R⁴ is an unsubstituted cyclopentyl. In embodiments, R⁴ is an unsubstituted cyclobutyl. In embodiments, R⁴ is an unsubstituted naphthyl. In embodiments, R⁴ is an unsubstituted 1-naphthyl. In embodiments, R⁴ is an unsubstituted 2-naphthyl. In embodiments, R⁴ is an unsubstituted 2-thienyl. In embodiments, R⁴ is an unsubstituted 3-thienyl. In embodiments, R⁴ is an unsubstituted 2-furanyl. In embodiments, R⁴ is an unsubstituted 3-furanyl. In embodiments, R⁴ is an unsubstituted 2-pyridyl. In embodiments, R⁴ is an unsubstituted 3-pyridyl. In embodiments, R⁴ is an unsubstituted 4-pyridyl. In embodiments, R⁴ is an unsubstituted 3-pyrazolyl. In embodiments, R⁴ is an unsubstituted 4-pyrazolyl. In embodiments, R⁴ is an unsubstituted 5-pyrazolyl. In embodiments, R⁴ is an unsubstituted 2-pyrrolyl. In embodiments, R⁴ is an unsubstituted 3-pyrrolyl. In embodiments, R⁴ is an unsubstituted 2-thiazolyl. In embodiments, R⁴ is an unsubstituted 4-thiazolyl. In embodiments, R⁴ is an unsubstituted 5-thiazolyl. In embodiments, R⁴ is an unsubstituted thiazolyl. In embodiments, R⁴ is substituted thiazolyl. In embodiments, R⁴ is methyl substituted thiazolyl. In embodiments, R⁴ is an unsubstituted thienyl. In embodiments, R⁴ is substituted thienyl. In embodiments, R⁴ is methyl substituted thienyl. In embodiments, R⁴ is an unsubstituted pyrazolyl. In embodiments, R⁴ is substituted pyrazolyl. In embodiments, R⁴ is methyl substituted pyrazolyl. In embodiments, R⁴ is an unsubstituted furanyl. In embodiments, R⁴ is substituted furanyl. In embodiments, R⁴ is methyl substituted furanyl.

In embodiments, R⁴ is independently halogen. In embodiments, R⁴ is independently —F. In embodiments, R⁴ is independently —Cl. In embodiments, R⁴ is independently —Br. In embodiments, R⁴ is independently —I. In embodiments, R⁴ is independently —CX⁴₃. In embodiments, R⁴ is independently —CHX⁴₂. In embodiments, R⁴ is independently —CH₂X⁴. In embodiments, R⁴ is independently —OCX⁴₃. In embodiments, R⁴ is independently —OCH₂X⁴. In embodiments, R⁴ is independently —OCHX⁴₂. In embodiments, R⁴ is independently —CN. In embodiments, R⁴ is independently —SOₙ₄R^{4D}. In embodiments, R⁴ is independently —SOᵥ₄NR^{4A}R^{4B}. In embodiments, R⁴ is independently —NHC(O)NR^{4A}R^{4B}. In embodiments, R⁴ is independently —N(O)ₘ₄. In embodiments, R⁴ is independently —NR^{4A}R^{4B}. In embodiments, R⁴ is independently —C(O)R^{4C}. In embodiments, R⁴ is independently —C(O)—OR^{4C}. In embodiments, R⁴ is independently —C(O)NR^{4A}R^{4B}. In embodiments, R⁴ is independently —OR^{4D}. In embodiments, R⁴ is independently —NR^{4A}SO₂R^{4D}. In embodiments, R⁴ is independently —NR^{4A}C(O)R^{4C}. In embodiments, R⁴ is independently —NR^{4A}C(O)OR^{4C}. In embodiments, R⁴ is independently —NR^{4A}OR^{4C}. In embodiments, R⁴ is independently —OH. In embodiments, R⁴ is independently —NH₂. In embodiments, R⁴ is independently —COOH. In embodiments, R⁴ is independently —CONH₂. In embodiments, R⁴ is independently —NO₂. In embodiments, R⁴ is independently —SH.

In embodiments, R⁴ is independently substituted or unsubstituted alkyl (e.g., C₁-C₈, C₁-C₆, or C₁-C₄). In embodiments, R⁴ is independently substituted alkyl (e.g., C₁-C₈, C₁-C₆, or C₁-C₄). In embodiments, R⁴ is independently unsubstituted alkyl (e.g., C₁-C₈, C₁-C₆, or C₁-C₄). In embodiments, R⁴ is independently substituted or unsubstituted heteroalkyl (e.g., 2 to 8 membered, 2 to 6 membered, or 2 to 4 membered). In embodiments, R⁴ is independently substituted heteroalkyl (e.g., 2 to 8 membered, 2 to 6 membered, or 2 to 4 membered). In embodiments, R⁴ is independently unsubstituted heteroalkyl (e.g., 2 to 8 membered, 2 to 6 membered, or 2 to 4 membered). In embodiments, R⁴ is independently substituted or unsubstituted cycloalkyl (e.g., C₃-C₈, C₃-C₆, or C₅-C₆). In embodiments, R⁴ is independently substituted cycloalkyl (e.g., C₃-C₈, C₃-C₆, or C₅-C₆). In embodiments, R⁴ is independently unsubstituted cycloalkyl (e.g., C₃-C₈, C₃-C₆, or C₅-C₆). In embodiments, R⁴ is independently substituted or unsubstituted heterocycloalkyl (e.g., 3 to 8 membered, 3 to 6 membered, or 5 to 6 membered). In embodiments, R⁴ is independently substituted heterocycloalkyl (e.g., 3 to 8 membered, 3 to 6 membered, or 5 to 6 membered). In embodiments, R⁴ is independently unsubstituted heterocycloalkyl (e.g., 3 to 8 membered, 3 to 6 membered, or 5 to 6 membered). In embodiments, R⁴ is independently substituted or unsubstituted aryl (e.g., C₆-C₁₀, C₁₀, or phenyl). In embodiments, R⁴ is independently substituted aryl (e.g., C₆-C₁₀, C₁₀, or phenyl). In embodiments, R⁴ is independently unsubstituted aryl (e.g., C₆-C₁₀, C₁₀, or phenyl). In embodiments, R⁴ is independently substituted or unsubstituted heteroaryl (e.g., 5 to 10 membered, 5 to 9 membered, or 5 to 6 membered). In embodiments, R⁴ is independently substituted heteroaryl (e.g., 5 to 10 membered, 5 to 9 membered, or 5 to 6 membered). In embodiments, R⁴ is independently unsubstituted heteroaryl (e.g., 5 to 10 membered, 5 to 9 membered, or 5 to 6 membered).

In embodiments, R^{4.1} is an unsubstituted methyl. In embodiments, R^{4.1} is —C(O)N(CH₃)₂. In embodiments, R^{4.1} is —CN. In embodiments, R^{4.1} is an unsubstituted methoxy. In embodiments, R^{4.1} is an unsubstituted tert-butyl. In embodiments, R^{4.1} is —OH. In embodiments, R^{4.1} is an unsubstituted ethoxy. In embodiments, R^{4.1} is —N(CH₃)₂. In embodiments, R^{4.1} is —SH. In embodiments, R^{4.1} is —SCH₃. In embodiments, R^{4.1} is —C(CN). In embodiments, R^{4.1} is —SCH₂CH₃. In embodiments, R^{4.1} is an unsubstituted ethyl. In embodiments, R^{4.1} is an unsubstituted propyl. In embodiments, R^{4.1} is an unsubstituted isopropyl. In embodiments, R^{4.1} is an unsubstituted butyl. In embodiments, R^{4.1} is an unsubstituted isobutyl. In embodiments, R^{4.1} is —NH₂. In embodiments, R^{4.1} is —NHCH₃. In embodiments, R^{4.1} is —NHCH₂CH₃. In embodiments, R^{4.1} is —N(CH₂CH₃)₂. In embodiments, R^{4.1} is —N(CH₃)(CH₂CH₃). In embodiments, R^{4.1} is halogen. In embodiments, R^{4.1} is —F. In embodiments, R^{4.1} is —Cl. In embodiments, R^{4.1} is —I. In embodiments, R^{4.1} is —Br. In embodiments, R^{4.1} is —C(O)NH₂. In embodiments, R^{4.1} is —C(O)NHCH₃. In embodiments, R^{4.1} is —C(O)NHCH₂CH₃. In embodiments, R^{4.1} is —C(O)N(CH₂CH₃)₂. In embodiments, R^{4.1} is —C(O)N(CH₃)(CH₂CH₃). In embodiments, R^{4.1} is —CF₃. In embodiments, R^{4.1} is —CHF₂. In embodiments, R^{4.1} is —CH₂F. In embodiments, R^{4.1} is —CCl₃. In embodiments, R^{4.1} is —CHCl₂. In embodiments, R^{4.1} is —CH₂Cl. In embodiments, R^{4.1} is —CBr₃. In embodiments, R^{4.1} is —CHBr₂. In embodiments, R^{4.1} is —CH₂Br. In embodiments, R^{4.1} is —CI₃. In embodiments, R^{4.1} is —CHI₂. In embodiments, R^{4.1} is —CH₂I.

In embodiments, R^{4.1} is an unsubstituted heteroalkyl. In embodiments, R^{4.1} is an unsubstituted 2 to 5 membered heteroalkyl. In embodiments, R^{4.1} is —OCH₃, —OCH₂CH₃, —N(CH₃)₂, —NH₂, —NH(CH₃), —N(CH₂CH₃)₂, —NH(CH₂CH₃), or —SH. In embodiments, R^{4.1} is —OCH₃. In embodiments, R^{4.1} is —OCH₂CH₃. In embodiments, R^{4.1} is —N(CH₃)₂. In embodiments, R^{4.1} is —NH₂. In embodiments, R^{4.1} is —NH(CH₃). In embodiments, R^{4.1} is —N(CH₂CH₃)₂. In embodiments, R^{4.1} is —NH(CH₂CH₃). In embodiments, R^{4.1} is —SH. In embodiments, R^{4.1} is —OCH₂CH₂CH₃. In embodiments, R^{4.1} is an unsubstituted methoxy. In embodiments, R^{4.1} is an unsubstituted ethoxy. In embodiments, R^{4.1} is an unsubstituted propoxy. In embodiments, R^{4.1} is an unsubstituted isopropoxy. In embodiments, R^{4.1} is an unsubstituted butoxy. In embodiments, R^{4.1} is an unsubstituted tert-butoxy. In embodiments, R^{4.1} is an unsubstituted pentoxy. In embodiments, R^{4.1} is an unsubstituted hexoxy.

In embodiments, R^{4.1} is an unsubstituted methoxy. In embodiments, R^{4.1} is an unsubstituted tert-butyl. In embodiments, R^{4.1} is an unsubstituted phenoxy. In embodiments, R^{4.1} is an unsubstituted methyl. In embodiments, R^{4.1} is —OH. In embodiments, R^{4.1} is an unsubstituted ethoxy. In embodiments, R^{4.1} is —N(CH₃)₂. In embodiments, R^{4.1} is —SH. In embodiments, R^{4.1} is —SCH₃. In embodiments, R^{4.1} is —SCH₂CH₃. In embodiments, R^{4.1} is an unsubstituted ethyl. In embodiments, R^{4.1} is an unsubstituted propyl. In embodiments, R^{4.1} is an unsubstituted isopropyl. In embodiments, R^{4.1} is an unsubstituted butyl. In embodiments, R^{4.1} is an unsubstituted isobutyl. In embodiments, R^{4.1} is —NH₂. In embodiments, R^{4.1} is —NHCH₃. In embodiments, R^{4.1} is —NHCH₂CH₃. In embodiments, R^{4.1} is —N(CH₂CH₃)₂. In embodiments, R^{4.1} is —N(CH₃)(CH₂CH₃). In embodiments, R^{4.1} is —OCH₃. In embodiments, R^{4.1} is an unsubstituted phenyl. In embodiments, R^{4.1} is —C(O)N(CH₃)₂. In embodiments, R^{4.1} is —C(O)NH(CH₃). In embodiments, R^{4.1} is —C(O)N(CH₂CH₃)₂. In embodiments, R^{4.1} is —C(O)NH(CH₂CH₃). In embodiments, R^{4.1} is an unsubstituted cyclohexyl. In embodiments, R^{4.1} is an unsubstituted morpholinyl. In embodiments, R^{4.1} is an unsubstituted piperazinyl. In embodiments, R^{4.1} is N-methyl substituted piperazinyl. In embodiments, R^{4.1} is an unsubstituted pyridyl. In embodiments, R^{4.1} is an unsubstituted cyclopentyl. In embodiments, R^{4.1} is an unsubstituted cyclobutyl. In embodiments, R^{4.1} is an unsubstituted naphthyl. In embodiments, R^{4.1} is an unsubstituted 1-naphthyl. In embodiments, R^{4.1} is an unsubstituted 2-naphthyl. In embodiments, R^{4.1} is an unsubstituted 2-thienyl. In embodiments, R^{4.1} is an unsubstituted 3-thienyl. In embodiments, R^{4.1} is an unsubstituted 2-furanyl. In embodiments, R^{4.1} is an unsubstituted 3-furanyl. In embodiments, R^{4.1} is an unsubstituted 2-pyridyl. In embodiments, R^{4.1} is an unsubstituted 3-pyridyl. In embodiments, R^{4.1} is an unsubstituted 4-pyridyl. In embodiments, R^{4.1} is an unsubstituted 3-pyrazolyl. In embodiments, R^{4.1} is an unsubstituted 4-pyrazolyl. In embodiments, R^{4.1} is an unsubstituted 5-pyrazolyl. In embodiments, R^{4.1} is an unsubstituted 2-pyrrolyl. In embodiments, R^{4.1} is an unsubstituted 3-pyrrolyl. In embodiments, R^{4.1} is an unsubstituted 2-thiazolyl. In embodiments, R^{4.1} is an unsubstituted 4-thiazolyl. In embodiments, R^{4.1} is an unsubstituted 5-thiazolyl. In embodiments, R^{4.1} is an unsubstituted thiazolyl. In embodiments, R^{4.1} is substituted thiazolyl. In embodiments, R^{4.1} is methyl substituted thiazolyl. In embodiments, R^{4.1} is an unsubstituted thienyl. In embodiments, R^{4.1} is substituted thienyl. In embodiments, R^{4.1} is methyl substituted thienyl. In embodiments, R^{4.1} is an unsubstituted pyrazolyl. In embodiments, R^{4.1} is substituted pyrazolyl. In embodiments, R^{4.1} is methyl substituted pyrazolyl. In embodiments, R^{4.1} is an unsubstituted furanyl. In embodiments, R^{4.1} is substituted furanyl. In embodiments, R^{4.1} is methyl substituted furanyl.

In embodiments, R^{4.1} is halogen. In embodiments, R^{4.1} is —F. In embodiments, R^{4.1} is —Cl. In embodiments, R^{4.1} is —Br. In embodiments, R^{4.1} is —I. In embodiments, R^{4.1} is —CX^{4.1}₃. In embodiments, R^{4.1} is —CHX^{4.1}₂. In embodiments, R^{4.1} is —CH₂X^{4.1}. In embodiments, R^{4.1} is —OCX^{4.1}₃. In embodiments, R^{4.1} is —OCH₂X^{4.1}. In embodiments, R^{4.1} is —OCHX^{4.1}₂. In embodiments, R^{4.1} is —CN. In embodiments, R^{4.1} is —SOₙ₄R^{4.1D}. In embodiments, R^{4.1} is —SOᵥ₄NR^{4.1A}R^{4.1B}. In embodiments, R^{4.1} is —NHC(O)NR^{4.1A}R^{4.1B}. In embodiments, R^{4.1} is —N(O)ₘ₄. In embodiments, R^{4.1} is —NR^{4.1A}R^{4.1B}. In embodiments, R^{4.1} is —C(O)R^{4.1C}. In embodiments, R^{4.1} is —C(O)—OR^{4.1C}. In embodiments, R^{4.1} is —C(O)NR^{4.1A}R^{4.1B}. In embodiments, R^{4.1} is —OR^{4.1D}. In embodiments, R^{4.1} is —NR^{4.1A}SO₂R^{4.1D}. In embodiments, R^{4.1} is —NR^{4.1A}C(O)R^{4.1C}. In embodiments, R^{4.1} is —NR^{4.1A}C(O)OR^{4.1C}. In embodiments, R^{4.1} is —NR^{4.1A}OR^{4.1C}. In embodiments, R^{4.1} is —OH. In embodiments, R^{4.1} is —NH₂. In embodiments, R^{4.1} is —COOH. In embodiments, R^{4.1} is —CONH₂. In embodiments, R^{4.1} is —NO₂. In embodiments, R^{4.1} is —SH.

In embodiments, R^{4.1} is substituted or unsubstituted alkyl (e.g., C₁-C₈, C₁-C₆, or C₁-C₄). In embodiments, R^{4.1} is substituted alkyl (e.g., C₁-C₈, C₁-C₆, or C₁-C₄). In embodiments, R^{4.1} is an unsubstituted alkyl (e.g., C₁-C₈, C₁-C₆, or C₁-C₄). In embodiments, R^{4.1} is substituted or unsubstituted heteroalkyl (e.g., 2 to 8 membered, 2 to 6 membered, or 2 to 4 membered). In embodiments, R^{4.1} is substituted heteroalkyl (e.g., 2 to 8 membered, 2 to 6 membered, or 2 to 4 membered). In embodiments, R^{4.1} is an unsubstituted heteroalkyl (e.g., 2 to 8 membered, 2 to 6 membered, or 2 to 4 membered). In embodiments, R^{4.1} is substituted or unsubstituted cycloalkyl (e.g., C₃-C₈, C₃-C₆, or C₅-C₆). In embodiments, R^{4.1} is substituted cycloalkyl (e.g., C₃-C₈, C₃-C₆, or C₅-C₆). In embodiments, R^{4.1} is an unsubstituted cycloalkyl (e.g., C₃-C₈, C₃-C₆, or C₅-C₆). In embodiments, R^{4.1} is substituted or unsubstituted heterocycloalkyl (e.g., 3 to 8 membered, 3 to 6 membered, or 5 to 6 membered). In embodiments, R^{4.1} is substituted heterocycloalkyl (e.g., 3 to 8 membered, 3 to 6 membered, or 5 to 6 membered). In embodiments, R^{4.1} is an unsubstituted heterocycloalkyl (e.g., 3 to 8 membered, 3 to 6 membered, or 5 to 6 membered). In embodiments, R^{4.1} is substituted or unsubstituted aryl (e.g., C₆-C₁₀, C₁₀, or phenyl). In embodiments, R^{4.1} is substituted aryl (e.g., C₆-C₁₀, C₁₀, or phenyl). In embodiments, R^{4.1} is an unsubstituted aryl (e.g., C₆-C₁₀, C₁₀, or phenyl). In embodiments, R^{4.1} is substituted or unsubstituted heteroaryl (e.g., 5 to 10 membered, 5 to 9 membered, or 5 to 6 membered). In embodiments, R^{4.1} is substituted heteroaryl (e.g., 5 to 10 membered, 5 to 9 membered, or 5 to 6 membered). In embodiments, R^{4.1} is an unsubstituted heteroaryl (e.g., 5 to 10 membered, 5 to 9 membered, or 5 to 6 membered).

In embodiments, R^{4.2} is an unsubstituted methyl. In embodiments, R^{4.2} is —C(O)N(CH₃)₂. In embodiments, R^{4.2} is —CN. In embodiments, R^{4.2} is an unsubstituted methoxy. In embodiments, R^{4.2} is an unsubstituted tert-butyl. In embodiments, R^{4.2} is —OH. In embodiments, R^{4.2} is an unsubstituted ethoxy. In embodiments, R^{4.2} is —N(CH₃)₂. In embodiments, R^{4.2} is —C(CN). In embodiments, R^{4.2} is —SH. In embodiments, R^{4.2} is —SCH₃. In embodiments, R^{4.2} is —SCH₂CH₃. In embodiments, R^{4.2} is an unsubstituted ethyl. In embodiments, R^{4.2} is an unsubstituted propyl. In embodiments, R^{4.2} is an unsubstituted isopropyl. In embodiments, R^{4.2} is an unsubstituted butyl. In embodiments, R^{4.2} is an unsubstituted isobutyl. In embodiments, R^{4.2} is —NH₂. In embodiments, R^{4.2} is —NHCH₃. In embodiments, R^{4.2} is —NHCH₂CH₃. In embodiments, R^{4.2} is —N(CH₂CH₃)₂. In embodiments, R^{4.2} is —N(CH₃)(CH₂CH₃). In embodiments, R^{4.2} is halogen. In embodiments, R^{4.2} is —F. In embodiments, R^{4.2} is —Cl. In embodiments, R^{4.2} is —I. In embodiments, R^{4.2} is —Br. In embodiments, R^{4.2} is —C(O)NH₂. In embodiments, R^{4.2} is —C(O)NHCH₃. In embodiments, R^{4.2} is —C(O)NHCH₂CH₃. In embodiments, R^{4.2} is —C(O)N(CH₂CH₃)₂. In embodiments, R^{4.2} is —C(O)N(CH₃)(CH₂CH₃). In embodiments, R^{4.2} is —CF₃. In embodiments, R^{4.2} is —CHF₂. In embodiments, R^{4.2} is —CH₂F. In embodiments, R^{4.2} is —CCl₃. In embodiments, R^{4.2} is —CHCl₂. In embodiments, R^{4.2} is —CH₂Cl. In embodiments, R^{4.2} is —CBr₃. In embodiments, R^{4.2} is —CHBr₂. In embodiments, R^{4.2} is —CH₂Br. In embodiments, R^{4.2} is —CI₃. In embodiments, R^{4.2} is —CHI₂. In embodiments, R^{4.2} is —CH₂I.

In embodiments, R^{4.2} is an unsubstituted heteroalkyl. In embodiments, R^{4.2} is an unsubstituted 2 to 5 membered heteroalkyl. In embodiments, R^{4.2} is —OCH₃, —OCH₂CH₃, —N(CH₃)₂, —NH₂, —NH(CH₃), —N(CH₂CH₃)₂, —NH(CH₂CH₃), or —SH. In embodiments, R^{4.2} is —OCH₃. In embodiments, R^{4.2} is —OCH₂CH₃. In embodiments, R^{4.2} is —N(CH₃)₂. In embodiments, R^{4.2} is —NH₂. In embodiments, R^{4.2} is —NH(CH₃). In embodiments, R^{4.2} is —N(CH₂CH₃)₂. In embodiments, R^{4.2} is —NH(CH₂CH₃). In embodiments, R^{4.2} is —SH. In embodiments, R^{4.2} is —OCH₂CH₂CH₃. In embodiments, R^{4.2} is an unsubstituted methoxy. In embodiments, R^{4.2} is an unsubstituted ethoxy. In embodiments, R^{4.2} is an unsubstituted propoxy. In embodiments, R^{4.2} is an unsubstituted isopropoxy. In embodiments, R^{4.2} is an unsubstituted butoxy. In embodiments, R^{4.2} is an unsubstituted tert-butoxy. In embodiments, R^{4.2} is an unsubstituted pentoxy. In embodiments, R^{4.2} is an unsubstituted hexoxy.

In embodiments, R^{4.2} is an unsubstituted methoxy. In embodiments, R^{4.2} is an unsubstituted tert-butyl. In embodiments, R^{4.2} is an unsubstituted phenoxy. In embodiments, R^{4.2} is an unsubstituted methyl. In embodiments, R^{4.2} is —OH. In embodiments, R^{4.2} is an unsubstituted ethoxy. In embodiments, R^{4.2} is —N(CH₃)₂. In embodiments, R^{4.2} is —SH. In embodiments, R^{4.2} is —SCH₃. In embodiments, R^{4.2} is —SCH₂CH₃. In embodiments, R^{4.2} is an unsubstituted ethyl. In embodiments, R^{4.2} is an unsubstituted propyl. In embodiments, R^{4.2} is an unsubstituted isopropyl. In embodiments, R^{4.2} is an unsubstituted butyl. In embodiments, R^{4.2} is an unsubstituted isobutyl. In embodiments, R^{4.2} is —NH₂. In embodiments, R^{4.2} is —NHCH₃. In embodiments, R^{4.2} is —NHCH₂CH₃. In embodiments, R^{4.2} is —N(CH₂CH₃)₂. In embodiments, R^{4.2} is —N(CH₃)(CH₂CH₃). In embodiments, R^{4.2} is —OCH₃. In embodiments, R^{4.2} is an unsubstituted phenyl. In embodiments, R^{4.2} is —C(O)N(CH₃)₂. In embodiments, R^{4.2} is —C(O)NH(CH₃). In embodiments, R^{4.2} is —C(O)N(CH₂CH₃)₂. In embodiments, R^{4.2} is —C(O)NH(CH₂CH₃). In embodiments, R^{4.2} is an unsubstituted cyclohexyl. In embodiments, R^{4.2} is an unsubstituted morpholinyl. In embodiments, R^{4.2} is an unsubstituted piperazinyl. In embodiments, R^{4.2} is N-methyl substituted piperazinyl. In embodiments, R^{4.2} is an unsubstituted pyridyl. In embodiments, R^{4.2} is an unsubstituted cyclopentyl. In embodiments, R^{4.2} is an unsubstituted cyclobutyl. In embodiments, R^{4.2} is an unsubstituted naphthyl. In embodiments, R^{4.2} is an unsubstituted 1-naphthyl. In embodiments, R^{4.2} is an unsubstituted 2-naphthyl. In embodiments, R^{4.2} is an unsubstituted 2-thienyl. In embodiments, R^{4.2} is an unsubstituted 3-thienyl. In embodiments, R^{4.2} is an unsubstituted 2-furanyl. In embodiments, R^{4.2} is an unsubstituted 3-furanyl. In embodiments, R^{4.2} is an unsubstituted 2-pyridyl. In embodiments, R^{4.2} is an unsubstituted 3-pyridyl. In embodiments, R^{4.2} is an unsubstituted 4-pyridyl. In embodiments, R^{4.2} is an unsubstituted 3-pyrazolyl. In embodiments, R^{4.2} is an unsubstituted 4-pyrazolyl. In embodiments, R^{4.2} is an unsubstituted 5-pyrazolyl. In embodiments, R^{4.2} is an unsubstituted 2-pyrrolyl. In embodiments, R^{4.2} is an unsubstituted 3-pyrrolyl. In embodiments, R^{4.2} is an unsubstituted 2-thiazolyl. In embodiments, R^{4.2} is an unsubstituted 4-thiazolyl. In embodiments, R^{4.2} is an unsubstituted 5-thiazolyl. In embodiments, R^{4.2} is an unsubstituted thiazolyl. In embodiments, R^{4.2} is substituted thiazolyl. In embodiments, R^{4.2} is methyl substituted thiazolyl. In embodiments, R^{4.2} is an unsubstituted thienyl. In embodiments, R^{4.2} is substituted thienyl. In embodiments, R^{4.2} is methyl substituted thienyl. In embodiments, R^{4.2} is an unsubstituted pyrazolyl. In embodiments, R^{4.2} is substituted pyrazolyl. In embodiments, R^{4.2} is methyl substituted pyrazolyl. In embodiments, R^{4.2} is an unsubstituted furanyl. In embodiments, R^{4.2} is substituted furanyl. In embodiments, R^{4.2} is methyl substituted furanyl.

In embodiments, R^{4.2} is halogen. In embodiments, R^{4.2} is —F. In embodiments, R^{4.2} is —Cl. In embodiments, R^{4.2} is —Br. In embodiments, R^{4.2} is —I. In embodiments, R^{4.2} is —CX^{4.2}₃. In embodiments, R^{4.2} is —CHX^{4.2}₂. In embodiments, R^{4.2} is —CH₂X^{4.2}. In embodiments, R^{4.2} is —OCX^{4.2}₃. In embodiments, R^{4.2} is —OCH₂X^{4.2}. In embodiments, R^{4.2} is —OCHX^{4.2}₂. In embodiments, R^{4.2} is —CN. In embodiments, R^{4.2} is —SOₙ₄R^{4.2D}. In embodiments, R^{4.2} is —SOᵥ₄NR^{4.2A}R^{4.2B}. In embodiments, R^{4.2} is —NHC(O)NR^{4.2A}R^{4.2B}. In embodiments, R^{4.2} is —N(O)ₘ₄. In embodiments, R^{4.2} is —NR^{4.2A}R^{4.2B}. In embodiments, R^{4.2} is —C(O)R^{4.2C}. In embodiments, R^{4.2} is —C(O)—OR^{4.2C}. In embodiments, R^{4.2} is —C(O)NR^{4.2A}R^{4.2B}. In embodiments, R^{4.2} is —OR^{4.2D}. In embodiments, R^{4.2} is —NR^{4.2A}SO₂R^{4.2D}. In embodiments, R^{4.2} is —NR^{4.2A}C(O)R^{4.2C}. In embodiments, R^{4.2} is —NR^{4.2A}C(O)OR^{4.2C}. In embodiments, R^{4.2} is —NR^{4.2A}OR^{4.2C}. In embodiments, R^{4.2} is —OH. In embodiments, R^{4.2} is —NH₂. In embodiments, R^{4.2} is —COOH. In embodiments, R^{4.2} is —CONH₂. In embodiments, R^{4.2} is —NO₂. In embodiments, R^{4.2} is —SH.

In embodiments, R^{4.2} is substituted or unsubstituted alkyl (e.g., C₁-C₈, C₁-C₆, or C₁-C₄). In embodiments, R^{4.2} is substituted alkyl (e.g., C₁-C₈, C₁-C₆, or C₁-C₄). In embodiments, R^{4.2} is an unsubstituted alkyl (e.g., C₁-C₈, C₁-C₆, or C₁-C₄). In embodiments, R^{4.2} is substituted or unsubstituted heteroalkyl (e.g., 2 to 8 membered, 2 to 6 membered, or 2 to 4 membered). In embodiments, R^{4.2} is substituted heteroalkyl (e.g., 2 to 8 membered, 2 to 6 membered, or 2 to 4 membered). In embodiments, R^{4.2} is an unsubstituted heteroalkyl (e.g., 2 to 8 membered, 2 to 6 membered, or 2 to 4 membered). In embodiments, R^{4.2} is substituted or unsubstituted cycloalkyl (e.g., C₃-C₈, C₃-C₆, or C₅-C₆). In embodiments, R^{4.2} is substituted cycloalkyl (e.g., C₃-C₈, C₃-C₆, or C₅-C₆). In embodiments, R^{4.2} is an unsubstituted cycloalkyl (e.g., C₃-C₈, C₃-C₆, or C₅-C₆). In embodiments, R^{4.2} is substituted or unsubstituted heterocycloalkyl (e.g., 3 to 8 membered, 3 to 6 membered, or 5 to 6 membered). In embodiments, R^{4.2} is substituted heterocycloalkyl (e.g., 3 to 8 membered, 3 to 6 membered, or 5 to 6 membered). In embodiments, R^{4.2} is an unsubstituted heterocycloalkyl (e.g., 3 to 8 membered, 3 to 6 membered, or 5 to 6 membered). In embodiments, R^{4.2} is substituted or unsubstituted aryl (e.g., C₆-C₁₀, C₁₀, or phenyl). In embodiments, R^{4.2} is substituted aryl (e.g., C₆-C₁₀, C₁₀, or phenyl). In embodiments, R^{4.2} is an unsubstituted aryl (e.g., C₆-C₁₀, C₁₀, or phenyl). In embodiments, R^{4.2} is substituted or unsubstituted heteroaryl (e.g., 5 to 10 membered, 5 to 9 membered, or 5 to 6 membered). In embodiments, R^{4.2} is substituted heteroaryl (e.g., 5 to 10 membered, 5 to 9 membered, or 5 to 6 membered). In embodiments, R^{4.2} is an unsubstituted heteroaryl (e.g., 5 to 10 membered, 5 to 9 membered, or 5 to 6 membered).

In embodiments, R^{4.3} is an unsubstituted methyl. In embodiments, R^{4.3} is —C(O)N(CH₃)₂. In embodiments, R^{4.3} is —CN. In embodiments, R^{4.3} is an unsubstituted methoxy. In embodiments, R^{4.3} is an unsubstituted tert-butyl. In embodiments, R^{4.3} is —OH. In embodiments, R^{4.3} is an unsubstituted ethoxy. In embodiments, R^{4.3} is —N(CH₃)₂. In embodiments, R^{4.3} is —C(CN). In embodiments, R^{4.3} is —SH. In embodiments, R^{4.3} is —SCH₃. In embodiments, R^{4.3} is —SCH₂CH₃. In embodiments, R^{4.3} is an unsubstituted ethyl. In embodiments, R^{4.3} is an unsubstituted propyl. In embodiments, R^{4.3} is an unsubstituted isopropyl. In embodiments, R^{4.3} is an unsubstituted butyl. In embodiments, R^{4.3} is an unsubstituted isobutyl. In embodiments, R^{4.3} is —NH₂. In embodiments, R^{4.3} is —NHCH₃. In embodiments, R^{4.3} is —NHCH₂CH₃. In embodiments, R^{4.3} is —N(CH₂CH₃)₂. In embodiments, R^{4.3} is —N(CH₃)(CH₂CH₃). In embodiments, R^{4.3} is halogen. In embodiments, R^{4.3} is —F. In embodiments, R^{4.3} is —Cl. In embodiments, R^{4.3} is —I. In embodiments, R^{4.3} is —Br. In embodiments, R^{4.3} is —C(O)NH₂. In embodiments, R^{4.3} is —C(O)NHCH₃. In embodiments, R^{4.3} is —C(O)NHCH₂CH₃. In embodiments, R^{4.3} is —C(O)N(CH₂CH₃)₂. In embodiments, R^{4.3} is —C(O)N(CH₃)(CH₂CH₃). In embodiments, R^{4.3} is —CF₃. In embodiments, R^{4.3} is —CHF₂. In embodiments, R^{4.3} is —CH₂F. In embodiments, R^{4.3} is —CCl₃. In embodiments, R^{4.3} is —CHCl₂. In embodiments, R^{4.3} is —CH₂Cl. In embodiments, R^{4.3} is —CBr₃. In embodiments, R^{4.3} is —CHBr₂. In embodiments, R^{4.3} is —CH₂Br. In embodiments, R^{4.3} is —CI₃. In embodiments, R^{4.3} is —CHI₂. In embodiments, R^{4.3} is —CH₂I.

In embodiments, R^{4.3} is an unsubstituted heteroalkyl. In embodiments, R^{4.3} is an unsubstituted 2 to 5 membered heteroalkyl. In embodiments, R^{4.3} is —OCH₃, —OCH₂CH₃, —N(CH₃)₂, —NH₂, —NH(CH₃), —N(CH₂CH₃)₂, —NH(CH₂CH₃), or —SH. In embodiments, R^{4.3} is —OCH₃. In embodiments, R^{4.3} is —OCH₂CH₃. In embodiments, R^{4.3} is —N(CH₃)₂. In embodiments, R^{4.3} is —NH₂. In embodiments, R^{4.3} is —NH(CH₃). In embodiments, R^{4.3} is —N(CH₂CH₃)₂. In embodiments, R^{4.3} is —NH(CH₂CH₃). In embodiments, R^{4.3} is —SH. In embodiments, R^{4.3} is —OCH₂CH₂CH₃. In embodiments, R^{4.3} is an unsubstituted methoxy. In embodiments, R^{4.3} is an unsubstituted ethoxy. In embodiments, R^{4.3} is an unsubstituted propoxy. In embodiments, R^{4.3} is an unsubstituted isopropoxy. In embodiments, R^{4.3} is an unsubstituted butoxy. In embodiments, R^{4.3} is an unsubstituted tert-butoxy. In embodiments, R^{4.3} is an unsubstituted pentoxy. In embodiments, R^{4.3} is an unsubstituted hexoxy.

In embodiments, R^{4.3} is an unsubstituted methoxy. In embodiments, R^{4.3} is an unsubstituted tert-butyl. In embodiments, R^{4.3} is an unsubstituted phenoxy. In embodiments, R^{4.3} is an unsubstituted methyl. In embodiments, R^{4.3} is —OH. In embodiments, R^{4.3} is an unsubstituted ethoxy. In embodiments, R^{4.3} is —N(CH₃)₂. In embodiments, R^{4.3} is —SH. In embodiments, R^{4.3} is —SCH₃. In embodiments, R^{4.3} is —SCH₂CH₃. In embodiments, R^{4.3} is an unsubstituted ethyl. In embodiments, R^{4.3} is an unsubstituted propyl. In embodiments, R^{4.3} is an unsubstituted isopropyl. In embodiments, R^{4.3} is an unsubstituted butyl. In embodiments, R^{4.3} is an unsubstituted isobutyl. In embodiments, R^{4.3} is —NH₂. In embodiments, R^{4.3} is —NHCH₃. In embodiments, R^{4.3} is —NHCH₂CH₃. In embodiments, R^{4.3} is —N(CH₂CH₃)₂. In embodiments, R^{4.3} is —N(CH₃)(CH₂CH₃). In embodiments, R^{4.3} is —OCH₃. In embodiments, R^{4.3} is an unsubstituted phenyl. In embodiments, R^{4.3} is —C(O)N(CH₃)₂. In embodiments, R^{4.3} is —C(O)NH(CH₃). In embodiments, R^{4.3} is —C(O)N(CH₂CH₃)₂. In embodiments, R^{4.3} is —C(O)NH(CH₂CH₃). In embodiments, R^{4.3} is an unsubstituted cyclohexyl. In embodiments, R^{4.3} is an unsubstituted morpholinyl. In embodiments, R^{4.3} is an unsubstituted piperazinyl. In embodiments, R^{4.3} is N-methyl substituted piperazinyl. In embodiments, R^{4.3} is an unsubstituted pyridyl. In embodiments, R^{4.3} is an unsubstituted cyclopentyl. In embodiments, R^{4.3} is an unsubstituted cyclobutyl. In embodiments, R^{4.3} is an unsubstituted naphthyl. In embodiments, R^{4.3} is an unsubstituted 1-naphthyl. In embodiments, R^{4.3} is an unsubstituted 2-naphthyl. In embodiments, R^{4.3} is an unsubstituted 2-thienyl. In embodiments, R^{4.3} is an unsubstituted 3-thienyl. In embodiments, R^{4.3} is an unsubstituted 2-furanyl. In embodiments, R^{4.3} is an unsubstituted 3-furanyl. In embodiments, R^{4.3} is an unsubstituted 2-pyridyl. In embodiments, R^{4.3} is an unsubstituted 3-pyridyl. In embodiments, R^{4.3} is an unsubstituted 4-pyridyl. In mbodiments, R^{4.3} is an unsubstituted 3-pyrazolyl. In embodiments, R^{4.3} is an unsubstituted 4-pyrazolyl. In embodiments, R^{4.3} is an unsubstituted 5-pyrazolyl. In embodiments, R^{4.3} is an unsubstituted 2-pyrrolyl. In embodiments, R^{4.3} is an unsubstituted 3-pyrrolyl. In embodiments, R^{4.3} is an unsubstituted 2-thiazolyl. In embodiments, R^{4.3} is an unsubstituted 4-thiazolyl. In embodiments, R^{4.3} is an unsubstituted 5-thiazolyl. In embodiments, R^{4.3} is an unsubstituted thiazolyl. In embodiments, R^{4.3} is substituted thiazolyl. In embodiments, R^{4.3} is methyl substituted thiazolyl. In embodiments, R^{4.3} is an unsubstituted thienyl. In embodiments, R^{4.3} is substituted thienyl. In embodiments, R^{4.3} is methyl substituted thienyl. In embodiments, R^{4.3} is an unsubstituted pyrazolyl. In embodiments, R^{4.3} is substituted pyrazolyl. In embodiments, R^{4.3} is methyl substituted pyrazolyl. In embodiments, R^{4.3} is an unsubstituted furanyl. In embodiments, R^{4.3} is substituted furanyl. In embodiments, R^{4.3} is methyl substituted furanyl.

In embodiments, R^{4.3} is halogen. In embodiments, R^{4.3} is —F. In embodiments, R^{4.3} is —Cl. In embodiments, R^{4.3} is —Br. In embodiments, R^{4.3} is —I. In embodiments, R^{4.3} is —CX^{4.3}₃. In embodiments, R^{4.3} is —CHX^{4.3}₂. In embodiments, R^{4.3} is —CH₂X^{4.3}. In embodiments, R^{4.3} is —OCX^{4.3}₃. In embodiments, R^{4.3} is —OCH₂X^{4.3}. In embodiments, R^{4.3} is —OCHX^{4.3}₂. In embodiments, R^{4.3} is —CN. In embodiments, R^{4.3} is —SOₙ₄R^{4.3D}. In embodiments, R^{4.3} is —SOᵥ₄NR^{4.3A}R^{4.3B}. In embodiments, R^{4.3} is —NHC(O)NR^{4.3A}R^{4.3B}. In embodiments, R^{4.3} is —N(O)ₘ₄. In embodiments, R^{4.3} is —NR^{4.3A}R^{4.3B}. In embodiments, R^{4.3} is —C(O)R^{4.3C}. In embodiments, R^{4.3} is —C(O)—OR^{4.3C}. In embodiments, R^{4.3} is —C(O)NR^{4.3A}R^{4.3B}. In embodiments, R^{4.3} is —OR^{4.3D}. In embodiments, R^{4.3} is —NR^{4.3A}SO₂R^{4.3D}. In embodiments, R^{4.3} is —NR^{4.3A}C(O)R^{4.3C}. In embodiments, R^{4.3} is —NR^{4.3A}C(O)OR^{4.3C}. In embodiments, R^{4.3} is —NR^{4.3A}OR^{4.3C}. In embodiments, R^{4.3} is —OH. In embodiments, R^{4.3} is —NH₂. In embodiments, R^{4.3} is —COOH. In embodiments, R^{4.3} is —CONH₂. In embodiments, R^{4.3} is —NO₂. In embodiments, R^{4.3} is —SH.

In embodiments, R^{4.3} is substituted or unsubstituted alkyl (e.g., C₁-C₈, C₁-C₆, or C₁-C₄). In embodiments, R^{4.3} is substituted alkyl (e.g., C₁-C₈, C₁-C₆, or C₁-C₄). In embodiments, R^{4.3} is an unsubstituted alkyl (e.g., C₁-C₈, C₁-C₆, or C₁-C₄). In embodiments, R^{4.3} is substituted or unsubstituted heteroalkyl (e.g., 2 to 8 membered, 2 to 6 membered, or 2 to 4 membered). In embodiments, R^{4.3} is substituted heteroalkyl (e.g., 2 to 8 membered, 2 to 6 membered, or 2 to 4 membered). In embodiments, R^{4.3} is an unsubstituted heteroalkyl (e.g., 2 to 8 membered, 2 to 6 membered, or 2 to 4 membered). In embodiments, R^{4.3} is substituted or unsubstituted cycloalkyl (e.g., C₃-C₈, C₃-C₆, or C₅-C₆). In embodiments, R^{4.3} is substituted cycloalkyl (e.g., C₃-C₈, C₃-C₆, or C₅-C₆). In embodiments, R^{4.3} is an unsubstituted cycloalkyl (e.g., C₃-C₈, C₃-C₆, or C₅-C₆). In embodiments, R^{4.3} is substituted or unsubstituted heterocycloalkyl (e.g., 3 to 8 membered, 3 to 6 membered, or 5 to 6 membered). In embodiments, R^{4.3} is substituted heterocycloalkyl (e.g., 3 to 8 membered, 3 to 6 membered, or 5 to 6 membered). In embodiments, R^{4.3} is an unsubstituted heterocycloalkyl (e.g., 3 to 8 membered, 3 to 6 membered, or 5 to 6 membered). In embodiments, R^{4.3} is substituted or unsubstituted aryl (e.g., C₆-C₁₀, C₁₀, or phenyl). In embodiments, R^{4.3} is substituted aryl (e.g., C₆-C₁₀, C₁₀, or phenyl). In embodiments, R^{4.3} is an unsubstituted aryl (e.g., C₆-C₁₀, C₁₀, or phenyl). In embodiments, R^{4.3} is substituted or unsubstituted heteroaryl (e.g., 5 to 10 membered, 5 to 9 membered, or 5 to 6 membered). In embodiments, R^{4.3} is substituted heteroaryl (e.g., 5 to 10 membered, 5 to 9 membered, or 5 to 6 membered). In embodiments, R^{4.3} is an unsubstituted heteroaryl (e.g., 5 to 10 membered, 5 to 9 membered, or 5 to 6 membered).

In embodiments, R^{4.4} is an unsubstituted methyl. In embodiments, R^{4.4} is —C(O)N(CH₃)₂. In embodiments, R^{4.4} is —CN. In embodiments, R^{4.4} is an unsubstituted methoxy. In embodiments, R^{4.4} is an unsubstituted tert-butyl. In embodiments, R^{4.4} is —OH. In embodiments, R^{4.4} is an unsubstituted ethoxy. In embodiments, R^{4.4} is —N(CH₃)₂. In embodiments, R^{4.4} is —C(CN). In embodiments, R^{4.4} is —SH. In embodiments, R^{4.4} is —SCH₃. In embodiments, R^{4.4} is —SCH₂CH₃. In embodiments, R^{4.4} is an unsubstituted ethyl. In embodiments, R^{4.4} is an unsubstituted propyl. In embodiments, R^{4.4} is an unsubstituted isopropyl. In embodiments, R^{4.4} is an unsubstituted butyl. In embodiments, R^{4.4} is an unsubstituted isobutyl. In embodiments, R^{4.4} is —NH₂. In embodiments, R^{4.4} is —NHCH₃. In embodiments, R^{4.4} is —NHCH₂CH₃. In embodiments, R^{4.4} is —N(CH₂CH₃)₂. In embodiments, R^{4.4} is —N(CH₃)(CH₂CH₃). In embodiments, R^{4.4} is halogen. In embodiments, R^{4.4} is —F. In embodiments, R^{4.4} is —Cl. In embodiments, R^{4.4} is —I. In embodiments, R^{4.4} is —Br. In embodiments, R^{4.4} is —C(O)NH₂. In embodiments, R^{4.4} is —C(O)NHCH₃. In embodiments, R^{4.4} is —C(O)NHCH₂CH₃. In embodiments, R^{4.4} is —C(O)N(CH₂CH₃)₂. In embodiments, R^{4.4} is —C(O)N(CH₃)(CH₂CH₃). In embodiments, R^{4.4} is —CF₃. In embodiments, R^{4.4} is —CHF₂. In embodiments, R^{4.4} is —CH₂F. In embodiments, R^{4.4} is —CCl₃. In embodiments, R^{4.4} is —CHCl₂. In embodiments, R^{4.4} is —CH₂Cl. In embodiments, R^{4.4} is —CBr₃. In embodiments, R^{4.4} is —CHBr₂. In embodiments, R^{4.4} is —CH₂Br. In embodiments, R^{4.4} is —CI₃. In embodiments, R^{4.4} is —CHI₂. In embodiments, R^{4.4} is —CH₂I.

In embodiments, R^{4.4} is an unsubstituted heteroalkyl. In embodiments, R^{4.4} is an unsubstituted 2 to 5 membered heteroalkyl. In embodiments, R^{4.4} is —OCH₃, —OCH₂CH₃, —N(CH₃)₂, —NH₂, —NH(CH₃), —N(CH₂CH₃)₂, —NH(CH₂CH₃), or —SH. In embodiments, R^{4.4} is —OCH₃. In embodiments, R^{4.4} is —OCH₂CH₃. In embodiments, R^{4.4} is —N(CH₃)₂. In embodiments, R^{4.4} is —NH₂. In embodiments, R^{4.4} is —NH(CH₃). In embodiments, R^{4.4} is —N(CH₂CH₃)₂. In embodiments, R^{4.4} is —NH(CH₂CH₃). In embodiments, R^{4.4} is —SH. In embodiments, R^{4.4} is —OCH₂CH₂CH₃. In embodiments, R^{4.4} is an unsubstituted methoxy. In embodiments, R^{4.4} is an unsubstituted ethoxy. In embodiments, R^{4.4} is an unsubstituted propoxy. In embodiments, R^{4.4} is an unsubstituted isopropoxy. In embodiments, R^{4.4} is an unsubstituted butoxy. In embodiments, R^{4.4} is an unsubstituted tert-butoxy. In embodiments, R^{4.4} is an unsubstituted pentoxy. In embodiments, R^{4.4} is an unsubstituted hexoxy.

In embodiments, R^{4.4} is an unsubstituted methoxy. In embodiments, R^{4.4} is an unsubstituted tert-butyl. In embodiments, R^{4.4} is an unsubstituted phenoxy. In embodiments, R^{4.4} is an unsubstituted methyl. In embodiments, R^{4.4} is —OH. In embodiments, R^{4.4} is an unsubstituted ethoxy. In embodiments, R^{4.4} is —N(CH₃)₂. In embodiments, R^{4.4} is —SH. In embodiments, R^{4.4} is —SCH₃. In embodiments, R^{4.4} is —SCH₂CH₃. In embodiments, R^{4.4} is an unsubstituted ethyl. In embodiments, R^{4.4} is an unsubstituted propyl. In embodiments, R^{4.4} is an unsubstituted isopropyl. In embodiments, R^{4.4} is an unsubstituted butyl. In embodiments, R^{4.4} is an unsubstituted isobutyl. In embodiments, R^{4.4} is —NH₂. In embodiments, R^{4.4} is —NHCH₃. In embodiments, R^{4.4} is —NHCH₂CH₃. In embodiments, R^{4.4} is —N(CH₂CH₃)₂. In embodiments, R^{4.4} is —N(CH₃)(CH₂CH₃). In embodiments, R^{4.4} is —OCH₃. In embodiments, R^{4.4} is an unsubstituted phenyl. In embodiments, R^{4.4} is —C(O)N(CH₃)₂. In embodiments, R^{4.4} is —C(O)NH(CH₃). In embodiments, R^{4.4} is —C(O)N(CH₂CH₃)₂. In embodiments, R^{4.4} is —C(O)NH(CH₂CH₃). In embodiments, R^{4.4} is an unsubstituted cyclohexyl. In embodiments, R^{4.4} is an unsubstituted morpholinyl. In embodiments, R^{4.4} is an unsubstituted piperazinyl. In embodiments, R^{4.4} is N-methyl substituted piperazinyl. In embodiments, R^{4.4} is an unsubstituted pyridyl. In embodiments, R^{4.4} is an unsubstituted cyclopentyl. In embodiments, R^{4.4} is an unsubstituted cyclobutyl. In embodiments, R^{4.4} is an unsubstituted naphthyl. In embodiments, R^{4.4} is an unsubstituted 1-naphthyl. In embodiments, R^{4.4} is an unsubstituted 2-naphthyl. In embodiments, R^{4.4} is an unsubstituted 2-thienyl. In embodiments, R^{4.4} is an unsubstituted 3-thienyl. In embodiments, R^{4.4} is an unsubstituted 2-furanyl. In embodiments, R^{4.4} is an unsubstituted 3-furanyl. In embodiments, R^{4.4} is an unsubstituted 2-pyridyl. In embodiments, R^{4.4} is an unsubstituted 3-pyridyl. In embodiments, R^{4.4} is an unsubstituted 4-pyridyl. In embodiments, R^{4.4} is an unsubstituted 3-pyrazolyl. In embodiments, R^{4.4} is an unsubstituted 4-pyrazolyl. In embodiments, R^{4.4} is an unsubstituted 5-pyrazolyl. In embodiments, R^{4.4} is an unsubstituted 2-pyrrolyl. In embodiments, R^{4.4} is an unsubstituted 3-pyrrolyl. In embodiments, R^{4.4} is an unsubstituted 2-thiazolyl. In embodiments, R^{4.4} is an unsubstituted 4-thiazolyl. In embodiments, R^{4.4} is an unsubstituted 5-thiazolyl. In embodiments, R^{4.4} is an unsubstituted thiazolyl. In embodiments, R^{4.4} is substituted thiazolyl. In embodiments, R^{4.4} is methyl substituted thiazolyl. In embodiments, R^{4.4} is an unsubstituted thienyl. In embodiments, R^{4.4} is substituted thienyl. In embodiments, R^{4.4} is methyl substituted thienyl. In embodiments, R^{4.4} is an unsubstituted pyrazolyl. In embodiments, R^{4.4} is substituted pyrazolyl. In embodiments, R^{4.4} is methyl substituted pyrazolyl. In embodiments, R^{4.4} is an unsubstituted furanyl. In embodiments, R^{4.4} is substituted furanyl. In embodiments, R^{4.4} is methyl substituted furanyl.

In embodiments, R^{4.4} is halogen. In embodiments, R^{4.4} is —F. In embodiments, R^{4.4} is —Cl. In embodiments, R^{4.4} is —Br. In embodiments, R^{4.4} is —I. In embodiments, R^{4.4} is —CX^{4.4}₃. In embodiments, R^{4.4} is —CHX^{4.4}₂. In embodiments, R^{4.4} is —CH₂X^{4.4}. In embodiments, R^{4.4} is —OCX^{4.4}₃. In embodiments, R^{4.4} is —OCH₂X^{4.4}. In embodiments, R^{4.4} is —OCHX^{4.4}₂. In embodiments, R^{4.4} is —CN. In embodiments, R^{4.4} is —SOₙ₄R^{4.4D}. In embodiments, R^{4.4} is —SOᵥ₄NR^{4.4A}R^{4.4B}. In embodiments, R^{4.4} is —NHC(O)NR^{4.4A}R^{4.4B}. In embodiments, R^{4.4} is —N(O)ₘ₄. In embodiments, R^{4.4} is —NR^{4.4A}R^{4.4B}. In embodiments, R^{4.4} is —C(O)R^{4.4C}. In embodiments, R^{4.4} is —C(O)—OR^{4.4C}. In embodiments, R^{4.4} is —C(O)NR^{4.4A}R^{4.4B}. In embodiments, R^{4.4} is —OR^{4.4D}. In embodiments, R^{4.4} is —NR^{4.4A}SO₂R^{4.4D}. In embodiments, R^{4.4} is —NR^{4.4A}C(O)R^{4.4C}. In embodiments, R^{4.4} is —NR^{4.4A}C(O)OR^{4.4C}. In embodiments, R^{4.4} is —NR^{4.4A}OR^{4.4C}. In embodiments, R^{4.4} is —OH. In embodiments, R^{4.4} is —NH₂. In embodiments, R^{4.4} is —COOH. In embodiments, R^{4.4} is —CONH₂. In embodiments, R^{4.4} is —NO₂. In embodiments, R^{4.4} is —SH.

In embodiments, R^{4.4} is substituted or unsubstituted alkyl (e.g., C₁-C₈, C₁-C₆, or C₁-C₄). In embodiments, R^{4.4} is substituted alkyl (e.g., C₁-C₈, C₁-C₆, or C₁-C₄). In embodiments, R^{4.4} is an unsubstituted alkyl (e.g., C₁-C₈, C₁-C₆, or C₁-C₄). In embodiments, R^{4.4} is substituted or unsubstituted heteroalkyl (e.g., 2 to 8 membered, 2 to 6 membered, or 2 to 4 membered). In embodiments, R^{4.4} is substituted heteroalkyl (e.g., 2 to 8 membered, 2 to 6 membered, or 2 to 4 membered). In embodiments, R^{4.4} is an unsubstituted heteroalkyl (e.g., 2 to 8 membered, 2 to 6 membered, or 2 to 4 membered). In embodiments, R^{4.4} is substituted or unsubstituted cycloalkyl (e.g., C₃-C₈, C₃-C₆, or C₅-C₆). In embodiments, R^{4.4} is substituted cycloalkyl (e.g., C₃-C₈, C₃-C₆, or C₅-C₆). In embodiments, R^{4.4} is an unsubstituted cycloalkyl (e.g., C₃-C₈, C₃-C₆, or C₅-C₆). In embodiments, R^{4.4} is substituted or unsubstituted heterocycloalkyl (e.g., 3 to 8 membered, 3 to 6 membered, or 5 to 6 membered). In embodiments, R^{4.4} is substituted heterocycloalkyl (e.g., 3 to 8 membered, 3 to 6 membered, or 5 to 6 membered). In embodiments, R^{4.4} is an unsubstituted heterocycloalkyl (e.g., 3 to 8 membered, 3 to 6 membered, or 5 to 6 membered). In embodiments, R^{4.4} is substituted or unsubstituted aryl (e.g., C₆-C₁₀, C₁₀, or phenyl). In embodiments, R^{4.4} is substituted aryl (e.g., C₆-C₁₀, C₁₀, or phenyl). In embodiments, R^{4.4} is an unsubstituted aryl (e.g., C₆-C₁₀, C₁₀, or phenyl). In embodiments, R^{4.4} is substituted or unsubstituted heteroaryl (e.g., 5 to 10 membered, 5 to 9 membered, or 5 to 6 membered). In embodiments, R^{4.4} is substituted heteroaryl (e.g., 5 to 10 membered, 5 to 9 membered, or 5 to 6 membered). In embodiments, R^{4.4} is an unsubstituted heteroaryl (e.g., 5 to 10 membered, 5 to 9 membered, or 5 to 6 membered).

In embodiments, R^{4.5} is an unsubstituted methyl. In embodiments, R^{4.5} is —C(O)N(CH₃)₂. In embodiments, R^{4.5} is —CN. In embodiments, R^{4.5} is an unsubstituted methoxy. In embodiments, R^{4.5} is an unsubstituted tert-butyl. In embodiments, R^{4.5} is —OH. In embodiments, R^{4.5} is an unsubstituted ethoxy. In embodiments, R^{4.5} is —N(CH₃)₂. In embodiments, R^{4.5} is —SH. In embodiments, R^{4.5} is —SCH₃. In embodiments, R^{4.5} is —SCH₂CH₃. In embodiments, R^{4.5} is an unsubstituted ethyl. In embodiments, R^{4.5} is an unsubstituted propyl. In embodiments, R^{4.5} is an unsubstituted isopropyl. In embodiments, R^{4.5} is an unsubstituted butyl. In embodiments, R^{4.5} is an unsubstituted isobutyl. In embodiments, R^{4.5} is —NH₂. In embodiments, R^{4.5} is —NHCH₃. In embodiments, R^{4.5} is —C(CN). In embodiments, R^{4.5} is —NHCH₂CH₃. In embodiments, R^{4.5} is —N(CH₂CH₃)₂. In embodiments, R^{4.5} is —N(CH₃)(CH₂CH₃). In embodiments, R^{4.5} is halogen. In embodiments, R^{4.5} is —F. In embodiments, R^{4.5} is —Cl. In embodiments, R^{4.5} is —I. In embodiments, R^{4.5} is —Br. In embodiments, R^{4.5} is —C(O)NH₂. In embodiments, R^{4.5} is —C(O)NHCH₃. In embodiments, R^{4.5} is —C(O)NHCH₂CH₃. In embodiments, R^{4.5} is —C(O)N(CH₂CH₃)₂. In embodiments, R^{4.5} is —C(O)N(CH₃)(CH₂CH₃). In embodiments, R^{4.5} is —CF₃. In embodiments, R^{4.5} is —CHF₂. In embodiments, R^{4.5} is —CH₂F. In embodiments, R^{4.5} is —CCl₃. In embodiments, R^{4.5} is —CHCl₂. In embodiments, R^{4.5} is —CH₂Cl. In embodiments, R^{4.5} is —CBr₃. In embodiments, R^{4.5} is —CHBr₂. In embodiments, R^{4.5} is —CH₂Br. In embodiments, R^{4.5} is —CI₃. In embodiments, R^{4.5} is —CHI₂. In embodiments, R^{4.5} is —CH₂I.

In embodiments, R^{4.5} is an unsubstituted heteroalkyl. In embodiments, R^{4.5} is an unsubstituted 2 to 5 membered heteroalkyl. In embodiments, R^{4.5} is —OCH₃, —OCH₂CH₃, —N(CH₃)₂, —NH₂, —NH(CH₃), —N(CH₂CH₃)₂, —NH(CH₂CH₃), or —SH. In embodiments, R^{4.5} is —OCH₃. In embodiments, R^{4.5} is —OCH₂CH₃. In embodiments, R^{4.5} is —N(CH₃)₂. In embodiments, R^{4.5} is —NH₂. In embodiments, R^{4.5} is —NH(CH₃). In embodiments, R^{4.5} is —N(CH₂CH₃)₂. In embodiments, R^{4.5} is —NH(CH₂CH₃). In embodiments, R^{4.5} is —SH. In embodiments, R^{4.5} is —OCH₂CH₂CH₃. In embodiments, R^{4.5} is an unsubstituted methoxy. In embodiments, R^{4.5} is an unsubstituted ethoxy. In embodiments, R^{4.5} is an unsubstituted propoxy. In embodiments, R^{4.5} is an unsubstituted isopropoxy. In embodiments, R^{4.5} is an unsubstituted butoxy. In embodiments, R^{4.5} is an unsubstituted tert-butoxy. In embodiments, R^{4.5} is an unsubstituted pentoxy. In embodiments, R^{4.5} is an unsubstituted hexoxy.

In embodiments, R^{4.5} is an unsubstituted methoxy. In embodiments, R^{4.5} is an unsubstituted tert-butyl. In embodiments, R^{4.5} is an unsubstituted phenoxy. In embodiments, R^{4.5} is an unsubstituted methyl. In embodiments, R^{4.5} is —OH. In embodiments, R^{4.5} is an unsubstituted ethoxy. In embodiments, R^{4.5} is —N(CH₃)₂. In embodiments, R^{4.5} is —SH. In embodiments, R^{4.5} is —SCH₃. In embodiments, R^{4.5} is —SCH₂CH₃. In embodiments, R^{4.5} is an unsubstituted ethyl. In embodiments, R^{4.5} is an unsubstituted propyl. In embodiments, R^{4.5} is an unsubstituted isopropyl. In embodiments, R^{4.5} is an unsubstituted butyl. In embodiments, R^{4.5} is an unsubstituted isobutyl. In embodiments, R^{4.5} is —NH₂. In embodiments, R^{4.5} is —NHCH₃. In embodiments, R^{4.5} is —NHCH₂CH₃. In embodiments, R^{4.5} is —N(CH₂CH₃)₂. In embodiments, R^{4.5} is —N(CH₃)(CH₂CH₃). In embodiments, R^{4.5} is —OCH₃. In embodiments, R^{4.5} is an unsubstituted phenyl. In embodiments, R^{4.5} is —C(O)N(CH₃)₂. In embodiments, R^{4.5} is —C(O)NH(CH₃). In embodiments, R^{4.5} is —C(O)N(CH₂CH₃)₂. In embodiments, R^{4.5} is —C(O)NH(CH₂CH₃). In embodiments, R^{4.5} is an unsubstituted cyclohexyl. In embodiments, R^{4.5} is an unsubstituted morpholinyl. In embodiments, R^{4.5} is an unsubstituted piperazinyl. In embodiments, R^{4.5} is N-methyl substituted piperazinyl. In embodiments, R^{4.5} is an unsubstituted pyridyl. In embodiments, R^{4.5} is an unsubstituted cyclopentyl. In embodiments, R^{4.5} is an unsubstituted cyclobutyl. In embodiments, R^{4.5} is an unsubstituted naphthyl. In embodiments, R^{4.5} is an unsubstituted 1-naphthyl. In embodiments, R^{4.5} is an unsubstituted 2-naphthyl. In embodiments, R^{4.5} is an unsubstituted 2-thienyl. In embodiments, R^{4.5} is an unsubstituted 3-thienyl. In embodiments, R^{4.5} is an unsubstituted 2-furanyl. In embodiments, R^{4.5} is an unsubstituted 3-furanyl. In embodiments, R^{4.5} is an unsubstituted 2-pyridyl. In embodiments, R^{4.5} is an unsubstituted 3-pyridyl. In embodiments, R^{4.5} is an unsubstituted 4-pyridyl. In embodiments, R^{4.5} is an unsubstituted 3-pyrazolyl. In embodiments, R^{4.5} is an unsubstituted 4-pyrazolyl. In embodiments, R^{4.5} is an unsubstituted 5-pyrazolyl. In embodiments, R^{4.5} is an unsubstituted 2-pyrrolyl. In embodiments, R^{4.5} is an unsubstituted 3-pyrrolyl. In embodiments, R^{4.5} is an unsubstituted 2-thiazolyl. In embodiments, R^{4.5} is an unsubstituted 4-thiazolyl. In embodiments, R^{4.5} is an unsubstituted 5-thiazolyl. In embodiments, R^{4.5} is an unsubstituted thiazolyl. In embodiments, R^{4.5} is substituted thiazolyl. In embodiments, R^{4.5} is methyl substituted thiazolyl. In embodiments, R^{4.5} is an unsubstituted thienyl. In embodiments, R^{4.5} is substituted thienyl. In embodiments, R^{4.5} is methyl substituted thienyl. In embodiments, R^{4.5} is an unsubstituted pyrazolyl. In embodiments, R^{4.5} is substituted pyrazolyl. In embodiments, R^{4.5} is methyl substituted pyrazolyl. In embodiments, R^{4.5} is an unsubstituted furanyl. In embodiments, R^{4.5} is substituted furanyl. In embodiments, R^{4.5} is methyl substituted furanyl.

In embodiments, R^{4.5} is halogen. In embodiments, R^{4.5} is —F. In embodiments, R^{4.5} is —Cl. In embodiments, R^{4.5} is —Br. In embodiments, R^{4.5} is —I. In embodiments, R^{4.5} is —CX^{4.5}₃. In embodiments, R^{4.5} is —CHX^{4.5}₂. In embodiments, R^{4.5} is —CH₂X^{4.5}. In embodiments, R^{4.5} is —OCX^{4.5}₃. In embodiments, R^{4.5} is —OCH₂X^{4.5}. In embodiments, R^{4.5} is —OCHX^{4.5}₂. In embodiments, R^{4.5} is —CN. In embodiments, R^{4.5} is —SOₙ₄R^{4.5D}. In embodiments, R^{4.5} is —SOᵥ₄NR^{4.5A}R^{4.5B}. In embodiments, R^{4.5} is —NHC(O)NR^{4.5A}R^{4.5B}. In embodiments, R^{4.5} is —N(O)ₘ₄. In embodiments, R^{4.5} is —NR^{4.5A}R^{4.5B}. In embodiments, R^{4.5} is —C(O)R^{4.5C}. In embodiments, R^{4.5} is —C(O)—OR^{4.5C}. In embodiments, R^{4.5} is —C(O)NR^{4.5A}R^{4.5B}. In embodiments, R^{4.5} is —OR^{4.5D}. In embodiments, R^{4.5} is —NR^{4.5A}SO₂R^{4.5D}. In embodiments, R^{4.5} is —NR^{4.5A}C(O)R^{4.5C}. In embodiments, R^{4.5} is —NR^{4.5A}C(O)OR^{4.5C}. In embodiments, R^{4.5} is —NR^{4.5A}OR^{4.5C}. In embodiments, R^{4.5} is —OH. In embodiments, R^{4.5} is —NH₂. In embodiments, R^{4.5} is —COOH. In embodiments, R^{4.5} is —CONH₂. In embodiments, R^{4.5} is —NO₂. In embodiments, R^{4.5} is —SH.

In embodiments, R^{4.5} is substituted or unsubstituted alkyl (e.g., C₁-C₈, C₁-C₆, or C₁-C₄). In embodiments, R^{4.5} is substituted alkyl (e.g., C₁-C₈, C₁-C₆, or C₁-C₄). In embodiments, R^{4.5} is an unsubstituted alkyl (e.g., C₁-C₈, C₁-C₆, or C₁-C₄). In embodiments, R^{4.5} is substituted or unsubstituted heteroalkyl (e.g., 2 to 8 membered, 2 to 6 membered, or 2 to 4 membered). In embodiments, R^{4.5} is substituted heteroalkyl (e.g., 2 to 8 membered, 2 to 6 membered, or 2 to 4 membered). In embodiments, R^{4.5} is an unsubstituted heteroalkyl (e.g., 2 to 8 membered, 2 to 6 membered, or 2 to 4 membered). In embodiments, R^{4.5} is substituted or unsubstituted cycloalkyl (e.g., C₃-C₈, C₃-C₆, or C₅-C₆). In embodiments, R^{4.5} is substituted cycloalkyl (e.g., C₃-C₈, C₃-C₆, or C₅-C₆). In embodiments, R^{4.5} is an unsubstituted cycloalkyl (e.g., C₃-C₈, C₃-C₆, or C₅-C₆). In embodiments, R^{4.5} is substituted or unsubstituted heterocycloalkyl (e.g., 3 to 8 membered, 3 to 6 membered, or 5 to 6 membered). In embodiments, R^{4.5} is substituted heterocycloalkyl (e.g., 3 to 8 membered, 3 to 6 membered, or 5 to 6 membered). In embodiments, R^{4.5} is an unsubstituted heterocycloalkyl (e.g., 3 to 8 membered, 3 to 6 membered, or 5 to 6 membered). In embodiments, R^{4.5} is substituted or unsubstituted aryl (e.g., C₆-C₁₀, C₁₀, or phenyl). In embodiments, R^{4.5} is substituted aryl (e.g., C₆-C₁₀, C₁₀, or phenyl). In embodiments, R^{4.5} is an unsubstituted aryl (e.g., C₆-C₁₀, C₁₀, or phenyl). In embodiments, R^{4.5} is substituted or unsubstituted heteroaryl (e.g., 5 to 10 membered, 5 to 9 membered, or 5 to 6 membered). In embodiments, R^{4.5} is substituted heteroaryl (e.g., 5 to 10 membered, 5 to 9 membered, or 5 to 6 membered). In embodiments, R^{4.5} is an unsubstituted heteroaryl (e.g., 5 to 10 membered, 5 to 9 membered, or 5 to 6 membered).

In embodiments, R^{4A} is independently hydrogen. In embodiments, R^{4A} is independently —CX^{4A}₃. In embodiments, R^{4A} is independently —CHX^{4A}₂. In embodiments, R^{4A} is independently —CH₂X^{4A}. In embodiments, R^{4A} is independently —CN. In embodiments, R^{4A} is independently —COOH. In embodiments, R^{4A} is independently —CONH₂.

In embodiments, R^{4A} is independently substituted or unsubstituted alkyl (e.g., C₁-C₈, C₁-C₆, or C₁-C₄). In embodiments, R^{4A} is independently substituted alkyl (e.g., C₁-C₈, C₁-C₆, or C₁-C₄). In embodiments, R^{4A} is independently unsubstituted alkyl (e.g., C₁-C₈, C₁-C₆, or C₁-C₄). In embodiments, R^{4A} is independently substituted or unsubstituted heteroalkyl (e.g., 2 to 8 membered, 2 to 6 membered, or 2 to 4 membered). In embodiments, R^{4A} is independently substituted heteroalkyl (e.g., 2 to 8 membered, 2 to 6 membered, or 2 to 4 membered). In embodiments, R^{4A} is independently unsubstituted heteroalkyl (e.g., 2 to 8 membered, 2 to 6 membered, or 2 to 4 membered). In embodiments, R^{4A} is independently substituted or unsubstituted cycloalkyl (e.g., C₃-C₈, C₃-C₆, or C₅-C₆). In embodiments, R^{4A} is independently substituted cycloalkyl (e.g., C₃-C₈, C₃-C₆, or C₅-C₆). In embodiments, R^{4A} is independently unsubstituted cycloalkyl (e.g., C₃-C₈, C₃-C₆, or C₅-C₆). In embodiments, R^{4A} is independently substituted or unsubstituted heterocycloalkyl (e.g., 3 to 8 membered, 3 to 6 membered, or 5 to 6 membered). In embodiments, R^{4A} is independently substituted heterocycloalkyl (e.g., 3 to 8 membered, 3 to 6 membered, or 5 to 6 membered). In embodiments, R^{4A} is independently unsubstituted heterocycloalkyl (e.g., 3 to 8 membered, 3 to 6 membered, or 5 to 6 membered). In embodiments, R^{4A} is independently substituted or unsubstituted aryl (e.g., C₆-C₁₀, C₁₀, or phenyl). In embodiments, R^{4A} is independently substituted aryl (e.g., C₆-C₁₀, C₁₀, or phenyl). In embodiments, R^{4A} is independently unsubstituted aryl (e.g., C₆-C₁₀, C₁₀, or phenyl). In embodiments, R^{4A} is independently substituted or unsubstituted heteroaryl (e.g., 5 to 10 membered, 5 to 9 membered, or 5 to 6 membered). In embodiments, R^{4A} is independently substituted heteroaryl (e.g., 5 to 10 membered, 5 to 9 membered, or 5 to 6 membered). In embodiments, R^{4A} is independently unsubstituted heteroaryl (e.g., 5 to 10 membered, 5 to 9 membered, or 5 to 6 membered). In embodiments, R^{4A} is independently unsubstituted methyl. In embodiments, R^{4A} is independently unsubstituted ethyl. In embodiments, R^{4A} is independently unsubstituted propyl. In embodiments, R^{4A} is independently unsubstituted isopropyl. In embodiments, R^{4A} is independently unsubstituted tert-butyl.

In embodiments, R^{4B} is independently hydrogen. In embodiments, R^{4B} is independently —CX^{4B}₃. In embodiments, R^{4B} is independently —CHX^{4B}₂. In embodiments, R^{4B} is independently —CH₂X^{4B}. In embodiments, R^{4B} is independently —CN. In embodiments, R^{4B} is independently —COOH. In embodiments, R^{4B} is independently —CONH₂.

In embodiments, R^{4B} is independently substituted or unsubstituted alkyl (e.g., C₁-C₈, C₁-C₆, or C₁-C₄). In embodiments, R^{4B} is independently substituted alkyl (e.g., C₁-C₈, C₁-C₆, or C₁-C₄). In embodiments, R^{4B} is independently unsubstituted alkyl (e.g., C₁-C₈, C₁-C₆, or C₁-C₄). In embodiments, R^{4B} is independently substituted or unsubstituted heteroalkyl (e.g., 2 to 8 membered, 2 to 6 membered, or 2 to 4 membered). In embodiments, R^{4B} is independently substituted heteroalkyl (e.g., 2 to 8 membered, 2 to 6 membered, or 2 to 4 membered). In embodiments, R^{4B} is independently unsubstituted heteroalkyl (e.g., 2 to 8 membered, 2 to 6 membered, or 2 to 4 membered). In embodiments, R^{4B} is independently substituted or unsubstituted cycloalkyl (e.g., C₃-C₈, C₃-C₆, or C₅-C₆). In embodiments, R^{4B} is independently substituted cycloalkyl (e.g., C₃-C₈, C₃-C₆, or C₅-C₆). In embodiments, R^{4B} is independently unsubstituted cycloalkyl (e.g., C₃-C₈, C₃-C₆, or C₅-C₆). In embodiments, R^{4B} is independently substituted or unsubstituted heterocycloalkyl (e.g., 3 to 8 membered, 3 to 6 membered, or 5 to 6 membered). In embodiments, R^{4B} is independently substituted heterocycloalkyl (e.g., 3 to 8 membered, 3 to 6 membered, or 5 to 6 membered). In embodiments, R^{4B} is independently unsubstituted heterocycloalkyl (e.g., 3 to 8 membered, 3 to 6 membered, or 5 to 6 membered). In embodiments, R^{4B} is independently substituted or unsubstituted aryl (e.g., C₆-C₁₀, C₁₀, or phenyl). In embodiments, R^{4B} is independently substituted aryl (e.g., C₆-C₁₀, C₁₀, or phenyl). In embodiments, R^{4B} is independently unsubstituted aryl (e.g., C₆-C₁₀, C₁₀, or phenyl). In embodiments, R^{4B} is independently substituted or unsubstituted heteroaryl (e.g., 5 to 10 membered, 5 to 9 membered, or 5 to 6 membered). In embodiments, R^{4B} is independently substituted heteroaryl (e.g., 5 to 10 membered, 5 to 9 membered, or 5 to 6 membered). In embodiments, R^{4B} is independently unsubstituted heteroaryl (e.g., 5 to 10 membered, 5 to 9 membered, or 5 to 6 membered). In embodiments, R^{4B} is independently unsubstituted methyl. In embodiments, R^{4B} is independently unsubstituted ethyl. In embodiments, R^{4B} is independently unsubstituted propyl. In embodiments, R^{4B} is independently unsubstituted isopropyl. In embodiments, R^{4B} is independently unsubstituted tert-butyl.

In embodiments, R^{4A} and R^{4B} substituents bonded to the same nitrogen atom may be joined to form a substituted or unsubstituted heterocycloalkyl. In embodiments, R^{4A} and R^{4B} substituents bonded to the same nitrogen atom may be joined to form a substituted or unsubstituted heteroaryl. In embodiments, R^{4A} and R^{4B} substituents bonded to the same nitrogen atom may be joined to form a substituted heterocycloalkyl. In embodiments, R^{4A} and R^{4B} substituents bonded to the same nitrogen atom may be joined to form a substituted heteroaryl. In embodiments, R^{4A} and R^{4B} substituents bonded to the same nitrogen atom may be joined to form an unsubstituted heterocycloalkyl. In embodiments, R^{4A} and R^{4B} substituents bonded to the same nitrogen atom may be joined to form an unsubstituted heteroaryl. In embodiments, R^{4A} and R^{4B} substituents bonded to the same nitrogen atom may be joined to form a substituted or unsubstituted 3 to 8 membered heterocycloalkyl. In embodiments, R^{4A} and R^{4B} substituents bonded to the same nitrogen atom may be joined to form a substituted or unsubstituted 5 to 10 membered heteroaryl. In embodiments, R^{4A} and R^{4B} substituents bonded to the same nitrogen atom may be joined to form a substituted 3 to 8 membered heterocycloalkyl. In embodiments, R^{4A} and R^{4B} substituents bonded to the same nitrogen atom may be joined to form a substituted 5 to 10 membered heteroaryl. In embodiments, R^{4A} and R^{4B} substituents bonded to the same nitrogen atom may be joined to form an unsubstituted 3 to 8 membered heterocycloalkyl. In embodiments, R^{4A} and R^{4B} substituents bonded to the same nitrogen atom may be joined to form an unsubstituted 5 to 10 membered heteroaryl. In embodiments, R^{4A} and R^{4B} substituents bonded to the same nitrogen atom may be joined to form a substituted or unsubstituted 3 to 6 membered heterocycloalkyl. In embodiments, R^{4A} and R^{4B} substituents bonded to the same nitrogen atom may be joined to form a substituted or unsubstituted 5 to 6 membered heteroaryl. In embodiments, R^{4A} and R^{4B} substituents bonded to the same nitrogen atom may be joined to form a substituted 3 to 6 membered heterocycloalkyl. In embodiments, R^{4A} and R^{4B} substituents bonded to the same nitrogen atom may be joined to form a substituted 5 to 6 membered heteroaryl. In embodiments, R^{4A} and R^{4B} substituents bonded to the same nitrogen atom may be joined to form an unsubstituted 3 to 6 membered heterocycloalkyl. In embodiments, R^{4A} and R^{4B} substituents bonded to the same nitrogen atom may be joined to form an unsubstituted 5 to 6 membered heteroaryl.

In embodiments, R^{4C} is independently hydrogen. In embodiments, R^{4C} is independently —CX^{4C}₃. In embodiments, R^{4C} is independently —CHX^{4C}₂. In embodiments, R^{4C} is independently —CH₂X^{4C}. In embodiments, R^{4C} is independently —CN. In embodiments, R^{4C} is independently —COOH. In embodiments, R^{4C} is independently —CONH₂.

In embodiments, R^{4C} is independently substituted or unsubstituted alkyl (e.g., C₁-C₈, C₁-C₆, or C₁-C₄). In embodiments, R^{4C} is independently substituted alkyl (e.g., C₁-C₈, C₁-C₆, or C₁-C₄). In embodiments, R^{4C} is independently unsubstituted alkyl (e.g., C₁-C₈, C₁-C₆, or C₁-C₄). In embodiments, R^{4C} is independently substituted or unsubstituted heteroalkyl (e.g., 2 to 8 membered, 2 to 6 membered, or 2 to 4 membered). In embodiments, R^{4C} is independently substituted heteroalkyl (e.g., 2 to 8 membered, 2 to 6 membered, or 2 to 4 membered). In embodiments, R^{4C} is independently unsubstituted heteroalkyl (e.g., 2 to 8 membered, 2 to 6 membered, or 2 to 4 membered). In embodiments, R^{4C} is independently substituted or unsubstituted cycloalkyl (e.g., C₃-C₈, C₃-C₆, or C₅-C₆). In embodiments, R^{4C} is independently substituted cycloalkyl (e.g., C₃-C₈, C₃-C₆, or C₅-C₆). In embodiments, R^{4C} is independently unsubstituted cycloalkyl (e.g., C₃-C₈, C₃-C₆, or C₅-C₆). In embodiments, R^{4C} is independently substituted or unsubstituted heterocycloalkyl (e.g., 3 to 8 membered, 3 to 6 membered, or 5 to 6 membered). In embodiments, R^{4C} is independently substituted heterocycloalkyl (e.g., 3 to 8 membered, 3 to 6 membered, or 5 to 6 membered). In embodiments, R^{4C} is independently unsubstituted heterocycloalkyl (e.g., 3 to 8 membered, 3 to 6 membered, or 5 to 6 membered). In embodiments, R^{4C} is independently substituted or unsubstituted aryl (e.g., C₆-C₁₀, C₁₀, or phenyl). In embodiments, R^{4C} is independently substituted aryl (e.g., C₆-C₁₀, C₁₀, or phenyl). In embodiments, R^{4C} is independently unsubstituted aryl (e.g., C₆-C₁₀, C₁₀, or phenyl). In embodiments, R^{4C} is independently substituted or unsubstituted heteroaryl (e.g., 5 to 10 membered, 5 to 9 membered, or 5 to 6 membered). In embodiments, R^{4C} is independently substituted heteroaryl (e.g., 5 to 10 membered, 5 to 9 membered, or 5 to 6 membered). In embodiments, R^{4C} is independently unsubstituted heteroaryl (e.g., 5 to 10 membered, 5 to 9 membered, or 5 to 6 membered). In embodiments, R^{4C} is independently unsubstituted methyl. In embodiments, R^{4C} is independently unsubstituted ethyl. In embodiments, R^{4C} is independently unsubstituted propyl. In embodiments, R^{4C} is independently unsubstituted isopropyl. In embodiments, R^{4C} is independently unsubstituted tert-butyl.

In embodiments, R^{4D} is independently hydrogen. In embodiments, R^{4D} is independently —CX^{4D}₃. In embodiments, R^{4D} is independently —CHX^{4D}₂. In embodiments, R^{4D} is independently —CH₂X^{4D}. In embodiments, R^{4D} is independently —CN. In embodiments, R^{4D} is independently —COOH. In embodiments, R^{4D} is independently —CONH₂.

In embodiments, R^{4D} is independently substituted or unsubstituted alkyl (e.g., C₁-C₈, C₁-C₆, or C₁-C₄). In embodiments, R^{4D} is independently substituted alkyl (e.g., C₁-C₈, C₁-C₆, or C₁-C₄). In embodiments, R^{4D} is independently unsubstituted alkyl (e.g., C₁-C₈, C₁-C₆, or C₁-C₄). In embodiments, R^{4D} is independently substituted or unsubstituted heteroalkyl (e.g., 2 to 8 membered, 2 to 6 membered, or 2 to 4 membered). In embodiments, R^{4D} is independently substituted heteroalkyl (e.g., 2 to 8 membered, 2 to 6 membered, or 2 to 4 membered). In embodiments, R^{4D} is independently unsubstituted heteroalkyl (e.g., 2 to 8 membered, 2 to 6 membered, or 2 to 4 membered). In embodiments, R^{4D} is independently substituted or unsubstituted cycloalkyl (e.g., C₃-C₈, C₃-C₆, or C₅-C₆). In embodiments, R^{4D} is independently substituted cycloalkyl (e.g., C₃-C₈, C₃-C₆, or C₅-C₆). In embodiments, R^{4D} is independently unsubstituted cycloalkyl (e.g., C₃-C₈, C₃-C₆, or C₅-C₆). In embodiments, R^{4D} is independently substituted or unsubstituted heterocycloalkyl (e.g., 3 to 8 membered, 3 to 6 membered, or 5 to 6 membered). In embodiments, R^{4D} is independently substituted heterocycloalkyl (e.g., 3 to 8 membered, 3 to 6 membered, or 5 to 6 membered). In embodiments, R^{4D} is independently unsubstituted heterocycloalkyl (e.g., 3 to 8 membered, 3 to 6 membered, or 5 to 6 membered). In embodiments, R^{4D} is independently substituted or unsubstituted aryl (e.g., C₆-C₁₀, C₁₀, or phenyl). In embodiments, R^{4D} is independently substituted aryl (e.g., C₆-C₁₀, C₁₀, or phenyl). In embodiments, R^{4D} is independently unsubstituted aryl (e.g., C₆-C₁₀, C₁₀, or phenyl). In embodiments, R^{4D} is independently substituted or unsubstituted heteroaryl (e.g., 5 to 10 membered, 5 to 9 membered, or 5 to 6 membered). In embodiments, R^{4D} is independently substituted heteroaryl (e.g., 5 to 10 membered, 5 to 9 membered, or 5 to 6 membered). In embodiments, R^{4D} is independently unsubstituted heteroaryl (e.g., 5 to 10 membered, 5 to 9 membered, or 5 to 6 membered). In embodiments, R^{4D} is independently unsubstituted methyl. In embodiments, R^{4D} is independently unsubstituted ethyl. In embodiments, R^{4D} is independently unsubstituted propyl. In embodiments, R^{4D} is independently unsubstituted isopropyl. In embodiments, R^{4D} is independently unsubstituted tert-butyl.

In embodiments, R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl. In embodiments, R⁴ is independently halogen, —CX⁴₃, —CHX⁴₂, —CH₂X⁴, —OCX⁴₃, —OCH₂X⁴, —OCHX⁴₂,—CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R²⁹-substituted or unsubstituted C₁-C₈ alkyl, R²⁹-substituted or unsubstituted 2 to 8 membered heteroalkyl, R²⁹-substituted or unsubstituted C₃-C₈ cycloalkyl, R²⁹-substituted or unsubstituted 3 to 6 membered heterocycloalkyl, R²⁹-substituted or unsubstituted phenyl, or R²⁹-substituted or unsubstituted 5 to 6 membered heteroaryl. X⁴ is —F, —Cl, —Br, or —I. In embodiments, R⁴ is independently hydrogen. In embodiments, R⁴ is independently methyl. In embodiments, R⁴ is independently ethyl.

R²⁹ is independently oxo, halogen, —CX²⁹₃, —CHX²⁹₂, —CH₂X²⁹, —OCX²⁹₃, —OCHX²⁹₂, —OCH₂X²⁹, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R³⁰-substituted or unsubstituted alkyl (e.g., C₁-C₈, C₁-C₆, or C₁-C₄), R³⁰-substituted or unsubstituted heteroalkyl (e.g., 2 to 8 membered, 2 to 6 membered, or 2 to 4 membered), R³⁰-substituted or unsubstituted cycloalkyl (e.g., C₃-C₈, C₃-C₆, or C₅-C₆), R³⁰-substituted or unsubstituted heterocycloalkyl (e.g., 3 to 8 membered, 3 to 6 membered, or 5 to 6 membered), R³⁰-substituted or unsubstituted aryl (e.g., C₆-C₁₀, C₁₀, or phenyl), or R³⁰-substituted or unsubstituted heteroaryl (e.g., 5 to 10 membered, 5 to 9 membered, or 5 to 6 membered). X²⁹ is —F, —Cl, —Br, or —I.

R³⁰ is independently oxo, halogen, —CX^{30C}₃, —CHX^{30C}₂, —CH₂X³⁰, —OCX^{30C}₃, —OCHX^{30C}₂, —OCH₂X³⁰, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R³¹-substituted or unsubstituted alkyl (e.g., C₁-C₈, C₁-C₆, or C₁-C₄), R³¹-substituted or unsubstituted heteroalkyl (e.g., 2 to 8 membered, 2 to 6 membered, or 2 to 4 membered), R³¹-substituted or unsubstituted cycloalkyl (e.g., C₃-C₈, C₃-C₆, or C₅-C₆), R³¹-substituted or unsubstituted heterocycloalkyl (e.g., 3 to 8 membered, 3 to 6 membered, or 5 to 6 membered), R³¹-substituted or unsubstituted aryl (e.g., C₆-C₁₀, C₁₀, or phenyl), or R³¹-substituted or unsubstituted heteroaryl (e.g., 5 to 10 membered, 5 to 9 membered, or 5 to 6 membered). X³⁰ is —F, —Cl, —Br, or —I.

In embodiments, R^{4A} is independently hydrogen, —CX^{4A}₃, —CN, —COOH, —CONH₂, —CHX^{4A}₂, —CH₂X^{4A}, R^{29A}-substituted or unsubstituted alkyl, R^{29A}-substituted or unsubstituted heteroalkyl, R^{29A}-substituted or unsubstituted cycloalkyl, R^{29A}-substituted or unsubstituted heterocycloalkyl, R^{29A}-substituted or unsubstituted aryl, or R^{29A}-substituted or unsubstituted heteroaryl. In embodiments, R^{4A} is independently hydrogen, —CX^{4A}₃, —CN, —COOH, —CONH₂, —CHX^{4A}₂, —CH²X^{4A}, R^{29A}-substituted or unsubstituted C₁-C₈ alkyl, R^{29A}-substituted or unsubstituted 2 to 8 membered heteroalkyl, R^{29A}-substituted or unsubstituted C₃-C₈ cycloalkyl, R^{29A}-substituted or unsubstituted 3 to 6 membered heterocycloalkyl, R^{29A}-substituted or unsubstituted phenyl, or R^{29A}-substituted or unsubstituted 5 to 6 membered heteroaryl. X^{4A} is —F, —Cl, —Br, or —I. In embodiments, R^{4A} is independently hydrogen. In embodiments, R^{4A} is independently methyl. In embodiments, R^{4A} is independently ethyl.

In embodiments, R^{4A} and R^{4B} substituents bonded to the same nitrogen atom may optionally be joined to form a R^{29A}-substituted or unsubstituted heterocycloalkyl or R^{29A}-substituted or unsubstituted heteroaryl. In embodiments, R^{4A} and R^{4B} substituents bonded to the same nitrogen atom may optionally be joined to form a R^{29A}-substituted or unsubstituted 3 to 6 membered heterocycloalkyl or R^{29A}-substituted or unsubstituted 5 to 6 membered heteroaryl.

R^{29A} is independently oxo, halogen, —CX^{29A}₃, —CHX^{29A}₂, —CH₂X^{29A}, —OCX^{29A}₃, —OCHX^{29A}₂, —OCH₂X^{29A}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{30A}-substituted or unsubstituted alkyl (e.g., C₁-C₈, C₁-C₆, or C₁-C₄), R^{30A}-substituted or unsubstituted heteroalkyl (e.g., 2 to 8 membered, 2 to 6 membered, or 2 to 4 membered), R^{30A}-substituted or unsubstituted cycloalkyl (e.g., C₃-C₈, C₃-C₆, or C₅-C₆), R^{30A}-substituted or unsubstituted heterocycloalkyl (e.g., 3 to 8 membered, 3 to 6 membered, or 5 to 6 membered), R^{30A}-substituted or unsubstituted aryl (e.g., C₆-C₁₀, C₁₀, or phenyl), or R^{30A}-substituted or unsubstituted heteroaryl (e.g., 5 to 10 membered, 5 to 9 membered, or 5 to 6 membered). X^{29A} is —F, —Cl, —Br, or —I.

R^{30A} is independently oxo, halogen, —CX^{30A}₃, —CHX^{30A}₂, —CH₂X^{30A}, —OCX^{30A}₃, —OCHX^{30A}₂, —OCH₂X^{30A}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{31A}-substituted or unsubstituted alkyl (e.g., C₁-C₈, C₁-C₆, or C₁-C₄), R^{31A}-substituted or unsubstituted heteroalkyl (e.g., 2 to 8 membered, 2 to 6 membered, or 2 to 4 membered), R^{31A}-substituted or unsubstituted cycloalkyl (e.g., C₃-C₈, C₃-C₆, or C₅-C₆), R^{31A}-substituted or unsubstituted heterocycloalkyl (e.g., 3 to 8 membered, 3 to 6 membered, or 5 to 6 membered), R^{31A}-substituted or unsubstituted aryl (e.g., C₆-C₁₀, C₁₀, or phenyl), or R^{31A}-substituted or unsubstituted heteroaryl (e.g., 5 to 10 membered, 5 to 9 membered, or 5 to 6 membered). X^{30A} is —F, —Cl, —Br, or —I.

In embodiments, R^{4B} is independently hydrogen, —CX^{4B}₃, —CN, —COOH, —CONH₂, —CHX^{4B}₂, —CH₂X^{4B}, R^{29B}-substituted or unsubstituted alkyl, R^{29B}-substituted or unsubstituted heteroalkyl, R^{29B}-substituted or unsubstituted cycloalkyl, R^{29B}-substituted or unsubstituted heterocycloalkyl, R^{29B}-substituted or unsubstituted aryl, or R^{29B}-substituted or unsubstituted heteroaryl. In embodiments, R^{4B} is independently hydrogen, —CX^{4B}₃, —CN, —COOH, —CONH₂, —CHX^{4B}₂, —CH₂X^{4B}, R^{29B}-substituted or unsubstituted C₁-C₈ alkyl, R^{29B}-substituted or unsubstituted 2 to 8 membered heteroalkyl, R^{29B}-substituted or unsubstituted C₃-C₈ cycloalkyl, R^{29B}-substituted or unsubstituted 3 to 6 membered heterocycloalkyl, R^{29B}-substituted or unsubstituted phenyl, or R^{29B}-substituted or unsubstituted 5 to 6 membered heteroaryl. X^{4B} is —F, —Cl, —Br, or —I. In embodiments, R^{4B} is independently hydrogen. In embodiments, R^{4B} is independently methyl. In embodiments, R^{4B} is independently ethyl.

In embodiments, R^{4A} and R^{4B} substituents bonded to the same nitrogen atom may optionally be joined to form a R^{29B}-substituted or unsubstituted heterocycloalkyl or R^{29B}-substituted or unsubstituted heteroaryl. In embodiments, R^{4A} and R^{4B} substituents bonded to the same nitrogen atom may optionally be joined to form a R^{29B}-substituted or unsubstituted 3 to 6 membered heterocycloalkyl or R^{29B}-substituted or unsubstituted 5 to 6 membered heteroaryl.

R^{29B} is independently oxo, halogen, —CX^{29B}₃, —CHX^{29B}₂, —CH₂X^{29B}, —OCX^{29B}₃, —OCHX^{29B}₂, —OCH₂X^{29B}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{30B}-substituted or unsubstituted alkyl (e.g., C₁-C₈, C₁-C₆, or C₁-C₄), R^{30B}-substituted or unsubstituted heteroalkyl (e.g., 2 to 8 membered, 2 to 6 membered, or 2 to 4 membered), R^{30B}-substituted or unsubstituted cycloalkyl (e.g., C₃-C₈, C₃-C₆, or C₅-C₆), R^{30B}-substituted or unsubstituted heterocycloalkyl (e.g., 3 to 8 membered, 3 to 6 membered, or 5 to 6 membered), R^{30B}-substituted or unsubstituted aryl (e.g., C₆-C₁₀, C₁₀, or phenyl), or R^{30B}-substituted or unsubstituted heteroaryl (e.g., 5 to 10 membered, 5 to 9 membered, or 5 to 6 membered). X^{29B} is —F, —Cl, —Br, or —I.

R^{30B} is independently oxo, halogen, —CX^{30B}₃, —CHX^{30B}₂, —CH₂X^{30B}, —OCX^{30B}₃, —OCHX^{30B}₂, —OCH₂X^{30B}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{31A}-substituted or unsubstituted alkyl (e.g., C₁-C₈, C₁-C₆, or C₁-C₄), R^{31A}-substituted or unsubstituted heteroalkyl (e.g., 2 to 8 membered, 2 to 6 membered, or 2 to 4 membered), R^{31A}-substituted or unsubstituted cycloalkyl (e.g., C₃-C₈, C₃-C₆, or C₅-C₆), R^{31A}-substituted or unsubstituted heterocycloalkyl (e.g., 3 to 8 membered, 3 to 6 membered, or 5 to 6 membered), R^{31A}-substituted or unsubstituted aryl (e.g., C₆-C₁₀, C₁₀, or phenyl), or R^{31A}-substituted or unsubstituted heteroaryl (e.g., 5 to 10 membered, 5 to 9 membered, or 5 to 6 membered). X^{30B} is —F, —Cl, —Br, or —I.

In embodiments, R^{4C} is independently hydrogen, —CX^{4C}₃, —CN, —COOH, —CONH₂, —CHX^{4C}₂, —CH₂X^{4C}, R^{29C}-substituted or unsubstituted alkyl, R^{29C}-substituted or unsubstituted heteroalkyl, R^{29C}-substituted or unsubstituted cycloalkyl, R^{29C}-substituted or unsubstituted heterocycloalkyl, R^{29C}-substituted or unsubstituted aryl, or R^{29C}-substituted or unsubstituted heteroaryl. In embodiments, R^{4C} is independently hydrogen, —CX^{4C}₃, —CN, —COOH, —CONH₂, —CHX^{4C}₂, —CH₂X^{4C}, R^{29C}-substituted or unsubstituted C₁-C₈ alkyl, R^{29C}-substituted or unsubstituted 2 to 8 membered heteroalkyl, R^{29C}-substituted or unsubstituted C₃-C₈ cycloalkyl, R^{29C}-substituted or unsubstituted 3 to 6 membered heterocycloalkyl, R^{29C}-substituted or unsubstituted phenyl, or R^{29C}-substituted or unsubstituted 5 to 6 membered heteroaryl. X^{4C} is —F, —Cl, —Br, or —I. In embodiments, R^{4C} is independently hydrogen. In embodiments, R^{4C} is independently methyl. In embodiments, R^{4C} is independently ethyl.

R^{29C} is independently oxo, halogen, —CX^{29C}₃, —CHX^{29C}₂, —CH₂X^{29C}, —OCX^{29C}₃, —OCHX^{29C}₂, —OCH₂X^{29C}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{30C}-substituted or unsubstituted alkyl (e.g., C₁-C₈, C₁-C₆, or C₁-C₄), R^{30C}-substituted or unsubstituted heteroalkyl (e.g., 2 to 8 membered, 2 to 6 membered, or 2 to 4 membered), R^{30C}-substituted or unsubstituted cycloalkyl (e.g., C₃-C₈, C₃-C₆, or C₅-C₆), R^{30C}-substituted or unsubstituted heterocycloalkyl (e.g., 3 to 8 membered, 3 to 6 membered, or 5 to 6 membered), R^{30C}-substituted or unsubstituted aryl (e.g., C₆-C₁₀, C₁₀, or phenyl), or R^{30C}-substituted or unsubstituted heteroaryl (e.g., 5 to 10 membered, 5 to 9 membered, or 5 to 6 membered). X^{29C} is —F, —Cl, —Br, or —I.

R^{30C} is independently oxo, halogen, —CX^{30C}₃, —CHX^{30C}₂, —CH₂X^{30C}, —OCX^{30C}₃, —OCHX^{30C}₂, —OCH₂X^{30C}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{31A}-substituted or unsubstituted alkyl (e.g., C₁-C₈, C₁-C₆, or C₁-C₄), R^{31A}-substituted or unsubstituted heteroalkyl (e.g., 2 to 8 membered, 2 to 6 membered, or 2 to 4 membered), R^{31A}-substituted or unsubstituted cycloalkyl (e.g., C₃-C₈, C₃-C₆, or C₅-C₆), R^{31A}-substituted or unsubstituted heterocycloalkyl (e.g., 3 to 8 membered, 3 to 6 membered, or 5 to 6 membered), R^{31A}-substituted or unsubstituted aryl (e.g., C₆-C₁₀, C₁₀, or phenyl), or R^{31A}-substituted or unsubstituted heteroaryl (e.g., 5 to 10 membered, 5 to 9 membered, or 5 to 6 membered). X^{30C} is —F, —Cl, —Br, or —I.

In embodiments, R^{4D} is independently hydrogen, —CX^{4D}₃, —CN, —COOH, —CONH₂, —CHX^{4D}₂, —CH₂X^{4D}, R^{29D}-substituted or unsubstituted alkyl, R^{29D}-substituted or unsubstituted heteroalkyl, R^{29D}-substituted or unsubstituted cycloalkyl, R^{29D}-substituted or unsubstituted heterocycloalkyl, R^{29D}-substituted or unsubstituted aryl, or R^{29D}-substituted or unsubstituted heteroaryl. In embodiments, R^{4D} is independently hydrogen, —CX^{4D}₃, —CN, —COOH, —CONH₂, —CHX^{4D}₂, —CH₂X^{4D}, R^{29D}-substituted or unsubstituted C₁-C₈ alkyl, R^{29D}-substituted or unsubstituted 2 to 8 membered heteroalkyl, R^{29D}-substituted or unsubstituted C₃-C₈ cycloalkyl, R^{29D}-substituted or unsubstituted 3 to 6 membered heterocycloalkyl, R^{29D}-substituted or unsubstituted phenyl, or R^{29D}-substituted or unsubstituted 5 to 6 membered heteroaryl. X^{4D} is —F, —Cl, —Br, or —I. In embodiments, R^{4D} is independently hydrogen. In embodiments, R^{4D} is independently methyl. In embodiments, R^{4D} is independently ethyl.

R^{29D} is independently oxo, halogen, —CX^{29D}₃, —CHX^{29D}₂, —CH₂X^{29D}, —OCX^{29D}₃, —OCHX^{29D}₂, —OCH₂X^{29D}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{30D}-substituted or unsubstituted alkyl (e.g., C₁-C₈, C₁-C₆, or C₁-C₄), R^{30D}-substituted or unsubstituted heteroalkyl (e.g., 2 to 8 membered, 2 to 6 membered, or 2 to 4 membered), R^{30D}-substituted or unsubstituted cycloalkyl (e.g., C₃-C₈, C₃-C₆, or C₅-C₆), R^{30D}-substituted or unsubstituted heterocycloalkyl (e.g., 3 to 8 membered, 3 to 6 membered, or 5 to 6 membered), R^{30D}-substituted or unsubstituted aryl (e.g., C₆-C₁₀, C₁₀, or phenyl), or R^{30D}-substituted or unsubstituted heteroaryl (e.g., 5 to 10 membered, 5 to 9 membered, or 5 to 6 membered). X^{29D} is —F, —Cl, —Br, or —I.

R^{30D} is independently oxo, halogen, —CX^{30D}₃, —CHX^{30D}₂, —CH₂X^{30D}, —OCX^{30D}₃, —OCHX^{30D}₂, —OCH₂X^{30D}, —CN, —OH, —NH₂, —COOH, —CONH₂, —NO₂, —SH, —SO₃H, —SO₄H, —NHNH₂, —ONH₂, —NHC(O)NHNH₂, —NHC(O)NH₂, —NHSO₂H, —NHC(O)H, —NHC(O)OH, —NHOH, R^{31A}-substituted or unsubstituted alkyl (e.g., C₁-C₈, C₁-C₆, or C₁-C₄), R^{31A}-substituted or unsubstituted heteroalkyl (e.g., 2 to 8 membered, 2 to 6 membered, or 2 to 4 membered), R^{31A}-substituted or unsubstituted cycloalkyl (e.g., C₃-C₈, C₃-C₆, or C₅-C₆), R^{31A}-substituted or unsubstituted heterocycloalkyl (e.g., 3 to 8 membered, 3 to 6 membered, or 5 to 6 membered), R^{31A}-substituted or unsubstituted aryl (e.g., C₆-C₁₀, C₁₀, or phenyl), or R^{31A}-substituted or unsubstituted heteroaryl (e.g., 5 to 10 membered, 5 to 9 membered, or 5 to 6 membered). X^{30D} is —F, —Cl, —Br, or —I.

R³¹, R^{31A}, R^{31B}, R^{31C}, and R^{31D} are independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, L¹ is a bond, —C(O)—, substituted or unsubstituted alkylene, or substituted or unsubstituted heteroalkylene. In embodiments, L¹ is a substituted or unsubstituted C₁-C₄ alkylene. In embodiments, L¹ is —C(O)CH₂CH₂CH₂—, —C(O)CH₂CH₂—, or —C(O)CH₂—.

In embodiments, L¹ is a bond, —S(O)₂—, —S(O)₂-Ph-, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene. In embodiments, L¹ is a bond. In embodiments, L¹ is a substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene. In embodiments, L¹ is an unsubstituted C₁-C₆ alkylene, unsubstituted 2 to 6 membered heteroalkylene, or unsubstituted C₃-C₆ cycloalkylene. In embodiments, L¹ is an unsubstituted methylene.

In embodiments, L¹ is a bond. In embodiments, L¹ is —S(O)₂—. In embodiments, L¹ is —S(O)₂-Ph-. In embodiments, L¹ is —NR⁶—. In embodiments, L¹ is —O—. In embodiments, L¹ is —S—. In embodiments, L¹ is —C(O)—. In embodiments, L¹ is —C(O)NR⁶—. In embodiments, L¹ is —NR⁶C(O)—. In embodiments, L¹ is —NR⁶C(O)NH—. In embodiments, L¹ is —NHC(O)NR⁶—. In embodiments, L¹ is —C(O)O—. In embodiments, L¹ is —OC(O)—. In embodiments, L¹ is —NH—. In embodiments, L¹ is —C(O)NH—. In embodiments, L¹ is —NHC(O)—. In embodiments, L¹ is —NHC(O)NH—. In embodiments, L¹ is —CH₂—. In embodiments, L¹ is —OCH₂—. In embodiments, L¹ is —CH₂O—. In embodiments, L¹ is —CH₂CH₂—. In embodiments, L¹ is —SCH₂—. In embodiments, L¹ is —CH₂S—. In embodiments, L¹ is —CHCH—. In embodiments, L¹ is —CC—. In embodiments, L¹ is —NHCH₂—. In embodiments, L¹ is —CH₂NH—.

In embodiments, L¹ is a substituted or unsubstituted alkylene. In embodiments, L¹ is a substituted or unsubstituted heteroalkylene. In embodiments, L¹ is a substituted or unsubstituted cycloalkylene. In embodiments, L¹ is a substituted or unsubstituted heterocycloalkylene. In embodiments, L¹ is a substituted or unsubstituted arylene. In embodiments, L¹ is a substituted or unsubstituted heteroarylene. In embodiments, L¹ is a substituted alkylene. In embodiments, L¹ is a substituted heteroalkylene. In embodiments, L¹ is a substituted cycloalkylene. In embodiments, L¹ is a substituted heterocycloalkylene. In embodiments, L¹ is a substituted arylene. In embodiments, L¹ is a substituted heteroarylene. In embodiments, L¹ is an unsubstituted alkylene. In embodiments, L¹ is an unsubstituted heteroalkylene. In embodiments, L¹ is an unsubstituted cycloalkylene. In embodiments, L¹ is an unsubstituted heterocycloalkylene. In embodiments, L¹ is an unsubstituted arylene. In embodiments, L¹ is an unsubstituted heteroarylene. In embodiments, L¹ is a substituted or unsubstituted C₁-C₈ alkylene. In embodiments, L¹ is a substituted or unsubstituted 2 to 8 membered heteroalkylene. In embodiments, L¹ is a substituted or unsubstituted C₃-C₈ cycloalkylene. In embodiments, L¹ is a substituted or unsubstituted 3 to 8 membered heterocycloalkylene. In embodiments, L¹ is a substituted or unsubstituted C₆-C₁₀ arylene. In embodiments, L¹ is a substituted or unsubstituted 5 to 10 membered heteroarylene. In embodiments, L¹ is a substituted or unsubstituted C₁-C₄ alkylene. In embodiments, L¹ is a substituted or unsubstituted 2 to 4 membered heteroalkylene. In embodiments, L¹ is a substituted or unsubstituted C₃-C₆ cycloalkylene. In embodiments, L¹ is a substituted or unsubstituted 3 to 6 membered heterocycloalkylene. In embodiments, L¹ is a substituted or unsubstituted phenylene. In embodiments, L¹ is a substituted or unsubstituted 5 to 6 membered heteroarylene.

In embodiments, L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, R⁴¹-substituted or unsubstituted alkylene, R⁴¹-substituted or unsubstituted heteroalkylene, R⁴¹-substituted or unsubstituted cycloalkylene, R⁴¹-substituted or unsubstituted heterocycloalkylene, R⁴¹-substituted or unsubstituted arylene, or R⁴¹-substituted or unsubstituted heteroarylene.

In embodiments, L¹ is a bond, —S(O)₂—, —S(O)₂-Ph-, —NH—, —O—, —S—, —C(O)—, —C(O)NH—, —NHC(O)—, —NHC(O)NH—, —C(O)O—, —OC(O)—, R⁴¹-substituted or unsubstituted alkylene, R⁴¹-substituted or unsubstituted heteroalkylene, R⁴¹-substituted or unsubstituted cycloalkylene, R⁴¹-substituted or unsubstituted heterocycloalkylene, R⁴¹-substituted or unsubstituted arylene, or R⁴¹-substituted or unsubstituted heteroarylene.

In embodiments, L¹ is a bond, —S(O)₂—, —S(O)₂-Ph-, —NH—, —O—, —S—, —C(O)—, —C(O)NH—, —NHC(O)—, —NHC(O)NH—, —C(O)O—, —OC(O)—, R⁴¹-substituted or unsubstituted C₁-C₈ alkylene, R⁴¹-substituted or unsubstituted 2 to 8 membered heteroalkylene, R⁴¹-substituted or unsubstituted C₃-C₈ cycloalkylene, R⁴¹-substituted or unsubstituted 3 to 6 membered heterocycloalkylene, R⁴¹-substituted or unsubstituted phenylene, or R⁴¹-substituted or unsubstituted 5 to 6 membered heteroarylene.

R⁴¹ is independently oxo, halogen, —CX⁴¹₃, —CHX⁴¹₂, —CH₂X⁴¹, —OCX⁴¹₃, —OCHX⁴¹₂, —OCH₂X⁴¹, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁴²-substituted or unsubstituted alkyl (e.g., C₁-C₈, C₁-C₆, or C₁-C₄), R⁴²-substituted or unsubstituted heteroalkyl (e.g., 2 to 8 membered, 2 to 6 membered, or 2 to 4 membered), R⁴²-substituted or unsubstituted cycloalkyl (e.g., C₃-C₈, C₃-C₆, or C₅-C₆), R⁴²-substituted or unsubstituted heterocycloalkyl (e.g., 3 to 8 membered, 3 to 6 membered, or 5 to 6 membered), R⁴²-substituted or unsubstituted aryl (e.g., C₆-C₁₀, C₁₀, or phenyl), or R⁴²-substituted or unsubstituted heteroaryl (e.g., 5 to 10 membered, 5 to 9 membered, or 5 to 6 membered). X⁴¹ is —F, —Cl, —Br, or —I.

R⁴² is independently oxo, halogen, —CX⁴²₃, —CHX⁴²₂, —CH₂X⁴², —OCX⁴²₃, —OCHX⁴²₂, —OCH₂X⁴², —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁴³-substituted or unsubstituted alkyl (e.g., C₁-C₈, C₁-C₆, or C₁-C₄), R⁴³-substituted or unsubstituted heteroalkyl (e.g., 2 to 8 membered, 2 to 6 membered, or 2 to 4 membered), R⁴³-substituted or unsubstituted cycloalkyl (e.g., C₃-C₈, C₃-C₆, or C₅-C₆), R⁴³-substituted or unsubstituted heterocycloalkyl (e.g., 3 to 8 membered, 3 to 6 membered, or 5 to 6 membered), R⁴³-substituted or unsubstituted aryl (e.g., C₆-C₁₀, C₁₀, or phenyl), or R⁴³-substituted or unsubstituted heteroaryl (e.g., 5 to 10 membered, 5 to 9 membered, or 5 to 6 membered). X⁴² is —F, —Cl, —Br, or —I.

R⁴³ is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, L¹ is a bond. In embodiments, L¹ is R⁴¹-substituted or unsubstituted C₁-C₂ alkylene. In embodiments, L¹ is R⁴¹-substituted or unsubstituted C₁-C₄ alkylene. In embodiments, L¹ is R⁴¹-substituted or unsubstituted C₁-C₆ alkylene. In embodiments, L¹ is R⁴¹-substituted or unsubstituted C₁-C₈ alkylene. In embodiments, L¹ is R⁴¹-substituted or unsubstituted alkylene (e.g., C₁-C₈ alkylene, C₁-C₆ alkylene, C₁-C₄ alkylene, C₁-C₂ alkylene). In embodiments, L¹ is R⁴¹-substituted C₁-C₂ alkylene. In embodiments, L¹ is R⁴¹-substituted C₁-C₄ alkylene. In embodiments, L¹ is R⁴¹-substituted C₁-C₆ alkylene. In embodiments, L¹ is R⁴¹-substituted C₁-C₈ alkylene. In embodiments, L¹ is R⁴¹-substituted alkylene (e.g., C₁-C₈ alkylene, C₁-C₆ alkylene, C₁-C₄ alkylene, C₁-C₂ alkylene). In embodiments, L¹ is R⁴¹-substituted methylene. In embodiments, L¹ is an unsubstituted C₁-C₂ alkylene. In embodiments, L¹ is an unsubstituted C₁-C₄ alkylene. In embodiments, L¹ is an unsubstituted C₁-C₆ alkylene. In embodiments, L¹ is an unsubstituted C₁-C₈ alkylene. In embodiments, L¹ is an unsubstituted alkylene (e.g., C₁-C₈ alkylene, C₁-C₆ alkylene, C₁-C₄ alkylene, C₁-C₂ alkylene). In embodiments, L¹ is R⁴¹-substituted or unsubstituted methylene. In embodiments, L¹ is R⁴¹-substituted methylene. In embodiments, L¹ is an unsubstituted methylene.

In embodiments, L¹ is R⁴¹-substituted or unsubstituted 2 to 4 membered heteroalkylene. In embodiments, L¹ is R⁴¹-substituted or unsubstituted 2 to 6 membered heteroalkylene. In embodiments, L¹ is R⁴¹-substituted or unsubstituted 2 to 8 membered heteroalkylene. In embodiments, L¹ is R⁴¹-substituted or unsubstituted heteroalkylene (e.g., 2 to 8 membered heteroalkylene, 2 to 6 membered heteroalkylene, 2 to 4 membered heteroalkylene). In embodiments, L¹ is R⁴¹-substituted 2 to 4 membered heteroalkylene. In embodiments, L¹ is R⁴¹-substituted 2 to 6 membered heteroalkylene. In embodiments, L¹ is R⁴¹-substituted 2 to 8 membered heteroalkylene. In embodiments, L¹ is R⁴¹-substituted heteroalkylene (e.g., 2 to 8 membered heteroalkylene, 2 to 6 membered heteroalkylene, 2 to 4 membered heteroalkylene). In embodiments, L¹ is an unsubstituted 2 to 4 membered heteroalkylene. In embodiments, L¹ is an unsubstituted 2 to 6 membered heteroalkylene. In embodiments, L¹ is an unsubstituted 2 to 8 membered heteroalkylene. In embodiments, L¹ is an unsubstituted heteroalkylene (e.g., 2 to 8 membered heteroalkylene, 2 to 6 membered heteroalkylene, 2 to 4 membered heteroalkylene).

In embodiments, L¹ is R⁴¹-substituted or unsubstituted ethylaminylene. In embodiments, L¹ is R⁴¹-substituted ethylaminylene. In embodiments, L¹ is an unsubstituted ethylaminylene. In embodiments, L¹ is R⁴¹-substituted or unsubstituted propylaminylene. In embodiments, L¹ is R⁴¹-substituted propyl aminylene. In embodiments, L¹ is an unsubstituted propylaminylene. In embodiments, L¹ is R⁴¹-substituted or unsubstituted butylaminylene. In embodiments, L¹ is R⁴¹-substituted butylaminylene. In embodiments, L¹ is an unsubstituted butylaminylene.

In embodiments, L¹ is R⁴¹-substituted or unsubstituted C₃-C₈ cycloalkylene. In embodiments, L¹ is R⁴¹-substituted or unsubstituted C₄-C₆ cycloalkylene. In embodiments, L¹ is R⁴¹-substituted or unsubstituted C₅-C₆ cycloalkylene. In embodiments, L¹ is R⁴¹-substituted or unsubstituted cyclocalkylene (e.g., C₃-C₈ cycloalkylene, C₄-C₆ cycloalkylene, or C₅-C₆ cycloalkylene). In embodiments, L¹ is R⁴¹-substituted C₃-C₈ cycloalkylene. In embodiments, L¹ is R⁴¹-substituted C₄-C₆ cycloalkylene. In embodiments, L¹ is R⁴¹-substituted C₅-C₆ cycloalkylene. In embodiments, L¹ is R⁴¹-substituted cyclocalkylene (e.g., C₃-C₈ cycloalkylene, C₄-C₆ cycloalkylene, or C₅-C₆ cycloalkylene). In embodiments, L¹ is an unsubstituted C₃-C₈ cycloalkylene. In embodiments, L¹ is an unsubstituted C₄-C₆ cycloalkylene. In embodiments, L¹ is an unsubstituted C₅-C₆ cycloalkylene. In embodiments, L¹ is an unsubstituted cycloalkylene (e.g., C₃-C₈ cycloalkylene, C₄-C₆ cycloalkylene, or C₅-C₆ cycloalkylene).

In embodiments, L¹ is R⁴¹-substituted or unsubstituted C₃-C₈ cycloalkylene. In embodiments, L¹ is R⁴¹-substituted or unsubstituted C₄-C₆ cycloalkylene. In embodiments, L¹ is R⁴¹-substituted or unsubstituted C₅-C₆ cycloalkylene. In embodiments, L¹ is R⁴¹-substituted or unsubstituted cyclocalkylene (e.g., C₃-C₈ cycloalkylene, C₄-C₆ cycloalkylene, or C₅-C₆ cycloalkylene). In embodiments, L¹ is R⁴¹-substituted C₃-C₈ cycloalkylene. In embodiments, L¹ is R⁴¹-substituted C₄-C₆ cycloalkylene. In embodiments, L¹ is R⁴¹-substituted C₅-C₆ cycloalkylene. In embodiments, L¹ is R⁴¹-substituted cycloalkylene (e.g., C₃-C₈ cycloalkylene, C₄-C₆ cycloalkylene, or C₅-C₆ cycloalkylene). In embodiments, L¹ is an unsubstituted C₃-C₈ cycloalkylene. In embodiments, L¹ is an unsubstituted C₄-C₆ cycloalkylene. In embodiments, L¹ is an unsubstituted C₅-C₆ cycloalkylene. In embodiments, L¹ is an unsubstituted cycloalkylene (e.g., C₃-C₈ cycloalkylene, C₄-C₆ cycloalkylene, or C₅-C₆ cycloalkylene).

In embodiments, L¹ is R⁴¹-substituted or unsubstituted 4 membered heterocycloalkylene. In embodiments, L¹ is R⁴¹-substituted or unsubstituted 5 membered heterocycloalkylene. In embodiments, L¹ is R⁴¹-substituted or unsubstituted 6 membered heterocycloalkylene. In embodiments, L¹ is R⁴¹-substituted or unsubstituted heterocycloalkylene (e.g., 3 to 6 membered heterocycloalkylene, 4 to 6 membered heterocycloalkylene, or 5 to 6 membered heterocycloalkylene). In embodiments, L¹ is R⁴¹-substituted 4 membered heterocycloalkylene. In embodiments, L¹ is R⁴¹-substituted 5 membered heterocycloalkylene. In embodiments, L¹ is R⁴¹-substituted 6 membered heterocycloalkylene. In embodiments, L¹ is R⁴¹-substituted heterocycloalkylene (e.g., 3 to 6 membered heterocycloalkylene, 4 to 6 membered heterocycloalkylene, or 5 to 6 membered heterocycloalkylene). In embodiments, L¹ is an unsubstituted 4 membered heterocycloalkylene. In embodiments, L¹ is an unsubstituted 5 membered heterocycloalkylene. In embodiments, L¹ unsubstituted 6 membered heterocycloalkylene. In embodiments, L¹ is an unsubstituted heterocycloalkylene (e.g., 3 to 6 membered heterocycloalkylene, 4 to 6 membered heterocycloalkylene, or 5 to 6 membered heterocycloalkylene).

In embodiments, L¹ is R⁴¹-substituted or unsubstituted arylene (e.g. C₆-C₁₀ arylene or C₆ arylene). In embodiments, L¹ is R⁴¹-substituted or unsubstituted C₆-C₁₀ arylene. In embodiments, L¹ is R⁴¹-substituted or unsubstituted C₆ arylene. In embodiments, L¹ is R⁴¹-substituted arylene (e.g. C₆-C₁₀ arylene or C₆ arylene). In embodiments, L¹ is R⁴¹-substituted C₆-C₁₀ arylene. In embodiments, L¹ is R⁴¹-substituted C₆ arylene. In embodiments, L¹ is an unsubstituted C₆-C₁₀ arylene. In embodiments, L¹ is an unsubstituted C₆ arylene.

In embodiments, L¹ is R⁴¹-substituted or unsubstituted heteroarylene (e.g. 5 to 10 membered heteroarylene, 5 to 9 membered heteroarylene, or 5 to 6 membered heteroarylene). In embodiments, L¹ is R⁴¹-substituted or unsubstituted 5 to 10 membered heteroarylene. In embodiments, L¹ is R⁴¹-substituted or unsubstituted 5 to 9 membered heteroarylene. In embodiments, L¹ is R⁴¹-substituted or unsubstituted 5 to 6 membered heteroarylene. In embodiments, L¹ is R⁴¹-substituted heteroarylene (e.g. 5 to 10 membered heteroarylene, 5 to 9 membered heteroarylene, or 5 to 6 membered heteroarylene). In embodiments, L¹ is R⁴¹-substituted 5 to 10 membered heteroarylene. In embodiments, L¹ is R⁴¹-substituted 5 to 9 membered heteroarylene. In embodiments, L¹ is R⁴¹-substituted 5 to 6 membered heteroarylene. In embodiments, L¹ is an unsubstituted heteroarylene (e.g. 5 to 10 membered heteroarylene, 5 to 9 membered heteroarylene, or 5 to 6 membered heteroarylene). In embodiments, L¹ is an unsubstituted 5 to 10 membered heteroarylene. In embodiments, L¹ is an unsubstituted 5 to 9 membered heteroarylene. In embodiments, L¹ is an unsubstituted 5 to 6 membered heteroarylene. In embodiments, L¹ is R⁴¹-substituted or unsubstituted indolinylene. In embodiments, L¹ is R⁴¹-substituted or unsubstituted indazolylene. In embodiments, L¹ is R⁴¹-substituted or unsubstituted benzimidazolylene. In embodiments, L¹ is R⁴¹-substituted or unsubstituted benzoxazolylene. In embodiments, L¹ is R⁴¹-substituted or unsubstituted azaindolylene. In embodiments, L¹ is R⁴¹-substituted substituted or unsubstituted purinylene. In embodiments, L¹ is R⁴¹-substituted or unsubstituted indolylene. In embodiments, L¹ is R⁴¹-substituted or unsubstituted pyrazinylene. In embodiments, L¹ is R⁴¹-substituted or unsubstituted pyrrolylene. In embodiments, L¹ is R⁴¹-substituted or unsubstituted imidazolylene. In embodiments, L¹ is R⁴¹-substituted or unsubstituted pyrazolylene. In embodiments, L¹ is R⁴¹-substituted or unsubstituted triazolylene. In embodiments, L¹ is R⁴¹-substituted or unsubstituted tetrazolylene. In embodiments, L¹ is R⁴¹-substituted or unsubstituted furanylene. In embodiments, L¹ is R⁴¹-substituted or unsubstituted thienylene.

In embodiments, R⁶ is independently hydrogen. In embodiments, R⁶ is independently halogen. In embodiments, R⁶ is independently —CX⁶₃. In embodiments, R⁶ is independently —CHX⁶₂. In embodiments, R⁶ is independently —CH₂X⁶. In embodiments, R⁶ is independently —OCX⁶₃. In embodiments, R⁶ is independently —OCH₂X⁶. In embodiments, R⁶ is independently —OCHX⁶₂. In embodiments, R⁶ is independently —CN. In embodiments, R⁶ is independently —SOₙ₆R^{6D}. In embodiments, R⁶ is independently —SOᵥ₆NR^{6A}R^{6B}. In embodiments, R⁶ is independently —NHC(O)NR^{6A}R^{6B}. In embodiments, R⁶ is independently —N(O)ₘ₆. In embodiments, R⁶ is independently —NR^{6A}R^{6B}. In embodiments, R⁶ is independently —C(O)R^{6C}. In embodiments, R⁶ is independently —C(O)—OR^{6C}. In embodiments, R⁶ is independently —C(O)NR^{6A}R^{6B}. In embodiments, R⁶ is independently —OR^{6D}. In embodiments, R⁶ is independently —NR^{6A}SO₂R^{6D}. In embodiments, R⁶ is independently —NR^{6A}C(O)R^{6C}. In embodiments, R⁶ is independently —NR^{6A}C(O)OR^{6C}. In embodiments, R⁶ is independently —NR^{6A}OR^{6C}. In embodiments, R⁶ is independently —OH. In embodiments, R⁶ is independently —NH₂. In embodiments, R⁶ is independently —COOH. In embodiments, R⁶ is independently —CONH₂. In embodiments, R⁶ is independently —NO₂. In embodiments, R⁶ is independently —SH.

In embodiments, R⁶ is independently substituted or unsubstituted alkyl (e.g., C₁-C₈, C₁-C₆, or C₁-C₄). In embodiments, R⁶ is independently substituted alkyl (e.g., C₁-C₈, C₁-C₆, or C₁-C₄). In embodiments, R⁶ is independently unsubstituted alkyl (e.g., C₁-C₈, C₁-C₆, or C₁-C₄). In embodiments, R⁶ is independently substituted or unsubstituted heteroalkyl (e.g., 2 to 8 membered, 2 to 6 membered, or 2 to 4 membered). In embodiments, R⁶ is independently substituted heteroalkyl (e.g., 2 to 8 membered, 2 to 6 membered, or 2 to 4 membered). In embodiments, R⁶ is independently unsubstituted heteroalkyl (e.g., 2 to 8 membered, 2 to 6 membered, or 2 to 4 membered). In embodiments, R⁶ is independently substituted or unsubstituted cycloalkyl (e.g., C₃-C₈, C₃-C₆, or C₅-C₆). In embodiments, R⁶ is independently substituted cycloalkyl (e.g., C₃-C₈, C₃-C₆, or C₅-C₆). In embodiments, R⁶ is independently unsubstituted cycloalkyl (e.g., C₃-C₈, C₃-C₆, or C₅-C₆). In embodiments, R⁶ is independently substituted or unsubstituted heterocycloalkyl (e.g., 3 to 8 membered, 3 to 6 membered, or 5 to 6 membered). In embodiments, R⁶ is independently substituted heterocycloalkyl (e.g., 3 to 8 membered, 3 to 6 membered, or 5 to 6 membered). In embodiments, R⁶ is independently unsubstituted heterocycloalkyl (e.g., 3 to 8 membered, 3 to 6 membered, or 5 to 6 membered). In embodiments, R⁶ is independently substituted or unsubstituted aryl (e.g., C₆-C₁₀, C₁₀, or phenyl). In embodiments, R⁶ is independently substituted aryl (e.g., C₆-C₁₀, C₁₀, or phenyl). In embodiments, R⁶ is independently unsubstituted aryl (e.g., C₆-C₁₀, C₁₀, or phenyl). In embodiments, R⁶ is independently substituted or unsubstituted heteroaryl (e.g., 5 to 10 membered, 5 to 9 membered, or 5 to 6 membered). In embodiments, R⁶ is independently substituted heteroaryl (e.g., 5 to 10 membered, 5 to 9 membered, or 5 to 6 membered). In embodiments, R⁶ is independently unsubstituted heteroaryl (e.g., 5 to 10 membered, 5 to 9 membered, or 5 to 6 membered).

In embodiments, R^{6A} is independently hydrogen. In embodiments, R^{6A} is independently —CX^{6A}₃. In embodiments, R^{6A} is independently —CHX^{6A}₂. In embodiments, R^{6A} is independently —CH₂X^{6A}. In embodiments, R^{6A} is independently —CN. In embodiments, R^{6A} is independently —COOH. In embodiments, R^{6A} is independently —CONH₂.

In embodiments, R^{6A} is independently substituted or unsubstituted alkyl (e.g., C₁-C₈, C₁-C₆, or C₁-C₄). In embodiments, R^{6A} is independently substituted alkyl (e.g., C₁-C₈, C₁-C₆, or C₁-C₄). In embodiments, R^{6A} is independently unsubstituted alkyl (e.g., C₁-C₈, C₁-C₆, or C₁-C₄). In embodiments, R^{6A} is independently substituted or unsubstituted heteroalkyl (e.g., 2 to 8 membered, 2 to 6 membered, or 2 to 4 membered). In embodiments, R^{6A} is independently substituted heteroalkyl (e.g., 2 to 8 membered, 2 to 6 membered, or 2 to 4 membered). In embodiments, R^{6A} is independently unsubstituted heteroalkyl (e.g., 2 to 8 membered, 2 to 6 membered, or 2 to 4 membered). In embodiments, R^{6A} is independently substituted or unsubstituted cycloalkyl (e.g., C₃-C₈, C₃-C₆, or C₅-C₆). In embodiments, R^{6A} is independently substituted cycloalkyl (e.g., C₃-C₈, C₃-C₆, or C₅-C₆). In embodiments, R^{6A} is independently unsubstituted cycloalkyl (e.g., C₃-C₈, C₃-C₆, or C₅-C₆). In embodiments, R^{6A} is independently substituted or unsubstituted heterocycloalkyl (e.g., 3 to 8 membered, 3 to 6 membered, or 5 to 6 membered). In embodiments, R^{6A} is independently substituted heterocycloalkyl (e.g., 3 to 8 membered, 3 to 6 membered, or 5 to 6 membered). In embodiments, R^{6A} is independently unsubstituted heterocycloalkyl (e.g., 3 to 8 membered, 3 to 6 membered, or 5 to 6 membered). In embodiments, R^{6A} is independently substituted or unsubstituted aryl (e.g., C₆-C₁₀, C₁₀, or phenyl). In embodiments, R^{6A} is independently substituted aryl (e.g., C₆-C₁₀, C₁₀, or phenyl). In embodiments, R^{6A} is independently unsubstituted aryl (e.g., C₆-C₁₀, C₁₀, or phenyl). In embodiments, R^{6A} is independently substituted or unsubstituted heteroaryl (e.g., 5 to 10 membered, 5 to 9 membered, or 5 to 6 membered). In embodiments, R^{6A} is independently substituted heteroaryl (e.g., 5 to 10 membered, 5 to 9 membered, or 5 to 6 membered). In embodiments, R^{6A} is independently unsubstituted heteroaryl (e.g., 5 to 10 membered, 5 to 9 membered, or 5 to 6 membered). In embodiments, R^{6A} is independently unsubstituted methyl. In embodiments, R^{6A} is independently unsubstituted ethyl. In embodiments, R^{6A} is independently unsubstituted propyl. In embodiments, R^{6A} is independently unsubstituted isopropyl. In embodiments, R^{6A} is independently unsubstituted tert-butyl.

In embodiments, R^{6B} is independently hydrogen. In embodiments, R^{6B} is independently —CX^{6B}₃. In embodiments, R^{6B} is independently —CHX^{6B}₂. In embodiments, R^{6B} is independently —CH₂X^{6B}. In embodiments, R^{6B} is independently —CN. In embodiments, R^{6B} is independently —COOH. In embodiments, R^{6B} is independently —CONH₂.

In embodiments, R^{6B} is independently substituted or unsubstituted alkyl (e.g., C₁-C₈, C₁-C₆, or C₁-C₄). In embodiments, R^{6B} is independently substituted alkyl (e.g., C₁-C₈, C₁-C₆, or C₁-C₄). In embodiments, R^{6B} is independently unsubstituted alkyl (e.g., C₁-C₈, C₁-C₆, or C₁-C₄). In embodiments, R^{6B} is independently substituted or unsubstituted heteroalkyl (e.g., 2 to 8 membered, 2 to 6 membered, or 2 to 4 membered). In embodiments, R^{6B} is independently substituted heteroalkyl (e.g., 2 to 8 membered, 2 to 6 membered, or 2 to 4 membered). In embodiments, R^{6B} is independently unsubstituted heteroalkyl (e.g., 2 to 8 membered, 2 to 6 membered, or 2 to 4 membered). In embodiments, R^{6B} is independently substituted or unsubstituted cycloalkyl (e.g., C₃-C₈, C₃-C₆, or C₅-C₆). In embodiments, R^{6B} is independently substituted cycloalkyl (e.g., C₃-C₈, C₃-C₆, or C₅-C₆). In embodiments, R^{6B} is independently unsubstituted cycloalkyl (e.g., C₃-C₈, C₃-C₆, or C₅-C₆). In embodiments, R^{6B} is independently substituted or unsubstituted heterocycloalkyl (e.g., 3 to 8 membered, 3 to 6 membered, or 5 to 6 membered). In embodiments, R^{6B} is independently substituted heterocycloalkyl (e.g., 3 to 8 membered, 3 to 6 membered, or 5 to 6 membered). In embodiments, R^{6B} is independently unsubstituted heterocycloalkyl (e.g., 3 to 8 membered, 3 to 6 membered, or 5 to 6 membered). In embodiments, R^{6B} is independently substituted or unsubstituted aryl (e.g., C₆-C₁₀, C₁₀, or phenyl). In embodiments, R^{6B} is independently substituted aryl (e.g., C₆-C₁₀, C₁₀, or phenyl). In embodiments, R^{6B} is independently unsubstituted aryl (e.g., C₆-C₁₀, C₁₀, or phenyl). In embodiments, R^{6B} is independently substituted or unsubstituted heteroaryl (e.g., 5 to 10 membered, 5 to 9 membered, or 5 to 6 membered). In embodiments, R^{6B} is independently substituted heteroaryl (e.g., 5 to 10 membered, 5 to 9 membered, or 5 to 6 membered). In embodiments, R^{6B} is independently unsubstituted heteroaryl (e.g., 5 to 10 membered, 5 to 9 membered, or 5 to 6 membered). In embodiments, R^{6B} is independently unsubstituted methyl. In embodiments, R^{6B} is independently unsubstituted ethyl. In embodiments, R^{6B} is independently unsubstituted propyl. In embodiments, R^{6B} is independently unsubstituted isopropyl. In embodiments, R^{6B} is independently unsubstituted tert-butyl.

In embodiments, R^{6A} and R^{6B} substituents bonded to the same nitrogen atom may be joined to form a substituted or unsubstituted heterocycloalkyl. In embodiments, R^{6A} and R^{6B} substituents bonded to the same nitrogen atom may be joined to form a substituted or unsubstituted heteroaryl. In embodiments, R^{6A} and R^{6B} substituents bonded to the same nitrogen atom may be joined to form a substituted heterocycloalkyl. In embodiments, R^{6A} and R^{6B} substituents bonded to the same nitrogen atom may be joined to form a substituted heteroaryl. In embodiments, R^{6A} and R^{6B} substituents bonded to the same nitrogen atom may be joined to form an unsubstituted heterocycloalkyl. In embodiments, R^{6A} and R^{6B} substituents bonded to the same nitrogen atom may be joined to form an unsubstituted heteroaryl. In embodiments, R^{6A} and R^{6B} substituents bonded to the same nitrogen atom may be joined to form a substituted or unsubstituted 3 to 8 membered heterocycloalkyl. In embodiments, R^{6A} and R^{6B} substituents bonded to the same nitrogen atom may be joined to form a substituted or unsubstituted 5 to 10 membered heteroaryl. In embodiments, R^{6A} and R^{6B} substituents bonded to the same nitrogen atom may be joined to form a substituted 3 to 8 membered heterocycloalkyl. In embodiments, R^{6A} and R^{6B} substituents bonded to the same nitrogen atom may be joined to form a substituted 5 to 10 membered heteroaryl. In embodiments, R^{6A} and R^{6B} substituents bonded to the same nitrogen atom may be joined to form an unsubstituted 3 to 8 membered heterocycloalkyl. In embodiments, R^{6A} and R^{6B} substituents bonded to the same nitrogen atom may be joined to form an unsubstituted 5 to 10 membered heteroaryl. In embodiments, R^{6A} and R^{6B} substituents bonded to the same nitrogen atom may be joined to form a substituted or unsubstituted 3 to 6 membered heterocycloalkyl. In embodiments, R^{6A} and R^{6B} substituents bonded to the same nitrogen atom may be joined to form a substituted or unsubstituted 5 to 6 membered heteroaryl. In embodiments, R^{6A} and R^{6B} substituents bonded to the same nitrogen atom may be joined to form a substituted 3 to 6 membered heterocycloalkyl. In embodiments, R^{6A} and R^{6B} substituents bonded to the same nitrogen atom may be joined to form a substituted 5 to 6 membered heteroaryl. In embodiments, R^{6A} and R^{6B} substituents bonded to the same nitrogen atom may be joined to form an unsubstituted 3 to 6 membered heterocycloalkyl. In embodiments, R^{6A} and R^{6B} substituents bonded to the same nitrogen atom may be joined to form an unsubstituted 5 to 6 membered heteroaryl.

In embodiments, R^{6C} is independently hydrogen. In embodiments, R^{6C} is independently —CX^{6C}₃. In embodiments, R^{6C} is independently —CHX^{6C}₂. In embodiments, R^{6C} is independently —CH₂X^{6C}. In embodiments, R^{6C} is independently —CN. In embodiments, R^{6C} is independently —COOH. In embodiments, R^{6C} is independently —CONH₂.

In embodiments, R^{6C} is independently substituted or unsubstituted alkyl (e.g., C₁-C₈, C₁-C₆, or C₁-C₄). In embodiments, R^{6C} is independently substituted alkyl (e.g., C₁-C₈, C₁-C₆, or C₁-C₄). In embodiments, R^{6C} is independently unsubstituted alkyl (e.g., C₁-C₈, C₁-C₆, or C₁-C₄). In embodiments, R^{6C} is independently substituted or unsubstituted heteroalkyl (e.g., 2 to 8 membered, 2 to 6 membered, or 2 to 4 membered). In embodiments, R^{6C} is independently substituted heteroalkyl (e.g., 2 to 8 membered, 2 to 6 membered, or 2 to 4 membered). In embodiments, R^{6C} is independently unsubstituted heteroalkyl (e.g., 2 to 8 membered, 2 to 6 membered, or 2 to 4 membered). In embodiments, R^{6C} is independently substituted or unsubstituted cycloalkyl (e.g., C₃-C₈, C₃-C₆, or C₅-C₆). In embodiments, R^{6C} is independently substituted cycloalkyl (e.g., C₃-C₈, C₃-C₆, or C₅-C₆). In embodiments, R^{6C} is independently unsubstituted cycloalkyl (e.g., C₃-C₈, C₃-C₆, or C₅-C₆). In embodiments, R^{6C} is independently substituted or unsubstituted heterocycloalkyl (e.g., 3 to 8 membered, 3 to 6 membered, or 5 to 6 membered). In embodiments, R^{6C} is independently substituted heterocycloalkyl (e.g., 3 to 8 membered, 3 to 6 membered, or 5 to 6 membered). In embodiments, R^{6C} is independently unsubstituted heterocycloalkyl (e.g., 3 to 8 membered, 3 to 6 membered, or 5 to 6 membered). In embodiments, R^{6C} is independently substituted or unsubstituted aryl (e.g., C₆-C₁₀, C₁₀, or phenyl). In embodiments, R^{6C} is independently substituted aryl (e.g., C₆-C₁₀, C₁₀, or phenyl). In embodiments, R^{6C} is independently unsubstituted aryl (e.g., C₆-C₁₀, C₁₀, or phenyl). In embodiments, R^{6C} is independently substituted or unsubstituted heteroaryl (e.g., 5 to 10 membered, 5 to 9 membered, or 5 to 6 membered). In embodiments, R^{6C} is independently substituted heteroaryl (e.g., 5 to 10 membered, 5 to 9 membered, or 5 to 6 membered). In embodiments, R^{6C} is independently unsubstituted heteroaryl (e.g., 5 to 10 membered, 5 to 9 membered, or 5 to 6 membered). In embodiments, R^{6C} is independently unsubstituted methyl. In embodiments, R^{6C} is independently unsubstituted ethyl. In embodiments, R^{6C} is independently unsubstituted propyl. In embodiments, R^{6C} is independently unsubstituted isopropyl. In embodiments, R^{6C} is independently unsubstituted tert-butyl.

In embodiments, R^{6D} is independently hydrogen. In embodiments, R^{6D} is independently —CX^{6D}₃. In embodiments, R^{6D} is independently —CHX^{6D}₂. In embodiments, R^{6D} is independently —CH₂X^{6D}. In embodiments, R^{6D} is independently —CN. In embodiments, R^{6D} is independently —COOH. In embodiments, R^{6D} is independently —CONH₂.

In embodiments, R^{6D} is independently substituted or unsubstituted alkyl (e.g., C₁-C₈, C₁-C₆, or C₁-C₄). In embodiments, R^{6D} is independently substituted alkyl (e.g., C₁-C₈, C₁-C₆, or C₁-C₄). In embodiments, R^{6D} is independently unsubstituted alkyl (e.g., C₁-C₈, C₁-C₆, or C₁-C₄). In embodiments, R^{6D} is independently substituted or unsubstituted heteroalkyl (e.g., 2 to 8 membered, 2 to 6 membered, or 2 to 4 membered). In embodiments, R^{6D} is independently substituted heteroalkyl (e.g., 2 to 8 membered, 2 to 6 membered, or 2 to 4 membered). In embodiments, R^{6D} is independently unsubstituted heteroalkyl (e.g., 2 to 8 membered, 2 to 6 membered, or 2 to 4 membered). In embodiments, R^{6D} is independently substituted or unsubstituted cycloalkyl (e.g., C₃-C₈, C₃-C₆, or C₅-C₆). In embodiments, R^{6D} is independently substituted cycloalkyl (e.g., C₃-C₈, C₃-C₆, or C₅-C₆). In embodiments, R^{6D} is independently unsubstituted cycloalkyl (e.g., C₃-C₈, C₃-C₆, or C₅-C₆). In embodiments, R^{6D} is independently substituted or unsubstituted heterocycloalkyl (e.g., 3 to 8 membered, 3 to 6 membered, or 5 to 6 membered). In embodiments, R^{6D} is independently substituted heterocycloalkyl (e.g., 3 to 8 membered, 3 to 6 membered, or 5 to 6 membered). In embodiments, R^{6D} is independently unsubstituted heterocycloalkyl (e.g., 3 to 8 membered, 3 to 6 membered, or 5 to 6 membered). In embodiments, R^{6D} is independently substituted or unsubstituted aryl (e.g., C₆-C₁₀, C₁₀, or phenyl). In embodiments, R^{6D} is independently substituted aryl (e.g., C₆-C₁₀, C₁₀, or phenyl). In embodiments, R^{6D} is independently unsubstituted aryl (e.g., C₆-C₁₀, C₁₀, or phenyl). In embodiments, R^{6D} is independently substituted or unsubstituted heteroaryl (e.g., 5 to 10 membered, 5 to 9 membered, or 5 to 6 membered). In embodiments, R^{6D} is independently substituted heteroaryl (e.g., 5 to 10 membered, 5 to 9 membered, or 5 to 6 membered). In embodiments, R^{6D} is independently unsubstituted heteroaryl (e.g., 5 to 10 membered, 5 to 9 membered, or 5 to 6 membered). In embodiments, R^{6D} is independently unsubstituted methyl. In embodiments, R^{6D} is independently unsubstituted ethyl. In embodiments, R^{6D} is independently unsubstituted propyl. In embodiments, R^{6D} is independently unsubstituted isopropyl. In embodiments, R^{6D} is independently unsubstituted tert-butyl.

In embodiments, R⁶ is independently hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}R^{6C}, R³⁵-substituted or unsubstituted alkyl, R³⁵-substituted or unsubstituted heteroalkyl, R³⁵-substituted or unsubstituted cycloalkyl, R³⁵-substituted or unsubstituted heterocycloalkyl, R³⁵-substituted or unsubstituted aryl, or R³⁵-substituted or unsubstituted heteroaryl. In embodiments, R⁶ is independently halogen, —CX⁶₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX⁶₃, —OCHX⁶₂, R³⁵-substituted or unsubstituted alkyl, R³⁵-substituted or unsubstituted heteroalkyl, R³⁵-substituted or unsubstituted cycloalkyl, R³⁵-substituted or unsubstituted heterocycloalkyl, R³⁵-substituted or unsubstituted aryl, or R³⁵-substituted or unsubstituted heteroaryl. In embodiments, R⁶ is independently halogen, —CX⁶₃, —CHX⁶₂, —CH₂X⁶, —OCX⁶₃, —OCHX²⁶₂, —OCH₂X⁶, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R³⁵-substituted or unsubstituted C₁-C₈ alkyl, R³⁵-substituted or unsubstituted 2 to 8 membered heteroalkyl, R³⁵-substituted or unsubstituted C₃-C₈ cycloalkyl, R³⁵-substituted or unsubstituted 3 to 6 membered heterocycloalkyl, R³⁵-substituted or unsubstituted phenyl, or R³⁵-substituted or unsubstituted 5 to 6 membered heteroaryl. X⁶ is —F, —Cl, —Br, or —I. In embodiments, R⁶ is independently hydrogen. In embodiments, R⁶ is independently methyl. In embodiments, R⁶ is independently ethyl.

R³⁵ is independently oxo, halogen, —CX³⁵₃, —CHX³⁵₂, —CH₂X³⁵, —OCX³⁵₃, —OCHX³⁵₂, —OCH₂X³⁵, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R³⁶-substituted or unsubstituted alkyl (e.g., C₁-C₈, C₁-C₆, or C₁-C₄), R³⁶-substituted or unsubstituted heteroalkyl (e.g., 2 to 8 membered, 2 to 6 membered, or 2 to 4 membered), R³⁶-substituted or unsubstituted cycloalkyl (e.g., C₃-C₈, C₃-C₆, or C₅-C₆), R³⁶-substituted or unsubstituted heterocycloalkyl (e.g., 3 to 8 membered, 3 to 6 membered, or 5 to 6 membered), R³⁶-substituted or unsubstituted aryl (e.g., C₆-C₁₀, C₁₀, or phenyl), or R³⁶-substituted or unsubstituted heteroaryl (e.g., 5 to 10 membered, 5 to 9 membered, or 5 to 6 membered). X³⁵ is —F, —Cl, —Br, or —I.

R³⁶ is independently oxo, halogen, —CX³⁶₃, —CHX³⁶₂, —CH₂X³⁶, —OCX³⁶₃, —OCHX³⁶₂, —OCH₂X³⁶, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R³⁷-substituted or unsubstituted alkyl (e.g., C₁-C₈, C₁-C₆, or C₁-C₄), R³⁷-substituted or unsubstituted heteroalkyl (e.g., 2 to 8 membered, 2 to 6 membered, or 2 to 4 membered), R³⁷-substituted or unsubstituted cycloalkyl (e.g., C₃-C₈, C₃-C₆, or C₅-C₆), R³⁷-substituted or unsubstituted heterocycloalkyl (e.g., 3 to 8 membered, 3 to 6 membered, or 5 to 6 membered), R³⁷-substituted or unsubstituted aryl (e.g., C₆-C₁₀, C₁₀, or phenyl), or R³⁷-substituted or unsubstituted heteroaryl (e.g., 5 to 10 membered, 5 to 9 membered, or 5 to 6 membered). X³⁶ is —F, —Cl, —Br, or —I.

In embodiments, R^{6A} is independently hydrogen, —CX^{6A}₃, —CN, —COOH, —CONH₂, —CHX^{6A}₂, —CH₂X^{6A}, R^{35A}-substituted or unsubstituted alkyl, R^{35A}-substituted or unsubstituted heteroalkyl, R^{35A}-substituted or unsubstituted cycloalkyl, R^{35A}-substituted or unsubstituted heterocycloalkyl, R^{35A}-substituted or unsubstituted aryl, or R^{35A}-substituted or unsubstituted heteroaryl. In embodiments, R^{6A} is independently hydrogen, —CX^{6A}₃, —COOH, —CONH₂, —CHX^{6A}₂, —CH₂X^{6A}, R^{35A}-substituted or unsubstituted C₁-C₈ alkyl, R^{35A}-substituted or unsubstituted 2 to 8 membered heteroalkyl, R^{35A}-substituted or unsubstituted C₃-C₈ cycloalkyl, R^{35A}-substituted or unsubstituted 3 to 6 membered heterocycloalkyl, R^{35A}-substituted or unsubstituted phenyl, or R^{35A}-substituted or unsubstituted 5 to 6 membered heteroaryl. X^{6A} is —F, —Cl, —Br, or —I. In embodiments, R^{6A} is independently hydrogen. In embodiments, R^{6A} is independently methyl. In embodiments, R^{6A} is independently ethyl.

In embodiments, R^{6A} and R^{6B} substituents bonded to the same nitrogen atom may optionally be joined to form a R^{35A}-substituted or unsubstituted heterocycloalkyl or R^{35A}-substituted or unsubstituted heteroaryl. In embodiments, R^{6A} and R^{6B} substituents bonded to the same nitrogen atom may optionally be joined to form a R^{35A}-substituted or unsubstituted 3 to 6 membered heterocycloalkyl or R^{35A}-substituted or unsubstituted 5 to 6 membered heteroaryl.

R^{35A} is independently oxo, halogen, —CX^{35A}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{35A}₃, —OCHX^{35A}₂, R^{36A}-substituted or unsubstituted alkyl (e.g., C₁-C₈, C₁-C₆, or C₁-C₄), R^{36A}-substituted or unsubstituted heteroalkyl (e.g., 2 to 8 membered, 2 to 6 membered, or 2 to 4 membered), R^{36A}-substituted or unsubstituted cycloalkyl (e.g., C₃-C C₃-C₆, or C₅-C₆), R^{36A}-substituted or unsubstituted heterocycloalkyl (e.g., 3 to 8 membered, 3 to 6 membered, or 5 to 6 membered), R^{36A}-substituted or unsubstituted aryl (e.g., C₆-C₁₀, C₁₀, or phenyl), or R^{36A}-substituted or unsubstituted heteroaryl (e.g., 5 to 10 membered, 5 to 9 membered, or 5 to 6 membered). X^{35A} is —F, —Cl, —Br, or —I.

R^{36A} is independently oxo, halogen, —CX^{36A}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{36A}₃, —OCHX^{36A}₂, R^{37A}-substituted or unsubstituted alkyl (e.g., C₁-C₈, C₁-C₆, or C₁-C₄), R^{37A}-substituted or unsubstituted heteroalkyl (e.g., 2 to 8 membered, 2 to 6 membered, or 2 to 4 membered), R^{37A}-substituted or unsubstituted cycloalkyl (e.g., C₃-C C₃-C₆, or C₅-C₆), R^{37A}-substituted or unsubstituted heterocycloalkyl (e.g., 3 to 8 membered, 3 to 6 membered, or 5 to 6 membered), R^{37A}-substituted or unsubstituted aryl (e.g., C₆-C₁₀, C₁₀, or phenyl), or R^{37A}-substituted or unsubstituted heteroaryl (e.g., 5 to 10 membered, 5 to 9 membered, or 5 to 6 membered). X^{36A} is —F, —Cl, —Br, or —I.

In embodiments, R^{6B} is independently hydrogen, —CX^{6B}₃, —CN, —COOH, —CONH₂, —CHX^{6B}₂, —CH₂X^{6B}, R^{35B}-substituted or unsubstituted alkyl, R^{35B}-substituted or unsubstituted heteroalkyl, R^{35B}-substituted or unsubstituted cycloalkyl, R^{35B}-substituted or unsubstituted heterocycloalkyl, R^{35B}-substituted or unsubstituted aryl, or R^{35B}-substituted or unsubstituted heteroaryl. In embodiments, R^{6B} is independently hydrogen, —CX^{6B}₃, —CN, —COOH, —CONH₂, —CHX^{6B}₂, —CH₂X^{6B}, R^{35B}-substituted or unsubstituted C₁-C₈ alkyl, R^{35B}-substituted or unsubstituted 2 to 8 membered heteroalkyl, R^{35B}-substituted or unsubstituted C₃-C₈ cycloalkyl, R^{35B}-substituted or unsubstituted 3 to 6 membered heterocycloalkyl, R^{35B}-substituted or unsubstituted phenyl, or R^{35B}-substituted or unsubstituted 5 to 6 membered heteroaryl. X^{6B} is —F, —Cl, —Br, or —I. In embodiments, R^{6B} is independently hydrogen. In embodiments, R^{6B} is independently methyl. In embodiments, R^{6B} is independently ethyl.

In embodiments, R^{6A} and R^{6B} substituents bonded to the same nitrogen atom may optionally be joined to form a R^{35B}-substituted or unsubstituted heterocycloalkyl or R^{35B}-substituted or unsubstituted heteroaryl. In embodiments, R^{6A} and R^{6B} substituents bonded to the same nitrogen atom may optionally be joined to form a R^{35B}-substituted or unsubstituted 3 to 6 membered heterocycloalkyl or R^{35B}-substituted or unsubstituted 5 to 6 membered heteroaryl.

R^{35B} is independently oxo, halogen, —CX^{35B}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{35B}₃, —OCHX^{35B}₂, R^{36B}-substituted or unsubstituted alkyl (e.g., C₁-C₈, C₁-C₆, or C₁-C₄), R^{36B}-substituted or unsubstituted heteroalkyl (e.g., 2 to 8 membered, 2 to 6 membered, or 2 to 4 membered), R^{36B}-substituted or unsubstituted cycloalkyl (e.g., C₃-C₈, C₃-C₆, or C₅-C₆), R^{36B}-substituted or unsubstituted heterocycloalkyl (e.g., 3 to 8 membered, 3 to 6 membered, or 5 to 6 membered), R^{36B}-substituted or unsubstituted aryl (e.g., C₆-C₁₀, C₁₀, or phenyl), or R^{36B}-substituted or unsubstituted heteroaryl (e.g., 5 to 10 membered, 5 to 9 membered, or 5 to 6 membered). X^{35B} is —F, —Cl, —Br, or —I.

R^{36B} is independently oxo, halogen, —CX^{36B}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{36B}₃, —OCHX^{36B}₂, R^{37B}-substituted or unsubstituted alkyl (e.g., C₁-C₈, C₁-C₆, or C₁-C₄), R^{37B}-substituted or unsubstituted heteroalkyl (e.g., 2 to 8 membered, 2 to 6 membered, or 2 to 4 membered), R^{37B}-substituted or unsubstituted cycloalkyl (e.g., C₃-C₈, C₃-C₆, or C₅-C₆), R^{37B}-substituted or unsubstituted heterocycloalkyl (e.g., 3 to 8 membered, 3 to 6 membered, or 5 to 6 membered), R^{37B}-substituted or unsubstituted aryl (e.g., C₆-C₁₀, C₁₀, or phenyl), or R^{37B}-substituted or unsubstituted heteroaryl (e.g., 5 to 10 membered, 5 to 9 membered, or 5 to 6 membered). X^{36B} is —F, —Cl, —Br, or —I.

In embodiments, R^{6C} is independently hydrogen, —CX^{6C}₃, —CN, —COOH, —CONH₂, —CHX^{6C}₂, —CH₂X^{6C}, R^{35C}-substituted or unsubstituted alkyl, R^{35C}-substituted or unsubstituted heteroalkyl, R^{35C}-substituted or unsubstituted cycloalkyl, R^{35C}-substituted or unsubstituted heterocycloalkyl, R^{35C}-substituted or unsubstituted aryl, or R^{35C}-substituted or unsubstituted heteroaryl. In embodiments, R^{6C} is independently hydrogen, —CX^{6C}₃, —CN, —COOH, —CONH₂, —CHX^{6C}₂, —CH₂X^{6C}, R^{35C}-substituted or unsubstituted C₁-C₈ alkyl, R^{35C}-substituted or unsubstituted 2 to 8 membered heteroalkyl, R^{35C}-substituted or unsubstituted C₃-C₈ cycloalkyl, R^{35C}-substituted or unsubstituted 3 to 6 membered heterocycloalkyl, R^{35C}-substituted or unsubstituted phenyl, or R^{35C}-substituted or unsubstituted 5 to 6 membered heteroaryl. X^{6C} is —F, —Cl, —Br, or —I. In embodiments, R^{6C} is independently hydrogen. In embodiments, R^{6C} is independently methyl. In embodiments, R^{6C} is independently ethyl.

R^{35C} is independently oxo, halogen, —CX^{35C}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{35C}₃, —OCHX^{35C}₂, R^{36C}-substituted or unsubstituted alkyl (e.g., C₁-C₈, C₁-C₆, or C₁-C₄), R^{36C}-substituted or unsubstituted heteroalkyl (e.g., 2 to 8 membered, 2 to 6 membered, or 2 to 4 membered), R^{36C}-substituted or unsubstituted cycloalkyl (e.g., C₃-C₈, C₃-C₆, or C₅-C₆), R^{36C}-substituted or unsubstituted heterocycloalkyl (e.g., 3 to 8 membered, 3 to 6 membered, or 5 to 6 membered), R^{36C}-substituted or unsubstituted aryl (e.g., C₆-C₁₀, C₁₀, or phenyl), or R^{36C}-substituted or unsubstituted heteroaryl (e.g., 5 to 10 membered, 5 to 9 membered, or 5 to 6 membered). X^{35C} is —F, —Cl, —Br, or —I.

R^{36C} is independently oxo, halogen, —CX^{36C}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{36C}₃, —OCHX^{36C}₂, R^{37C}-substituted or unsubstituted alkyl (e.g., C₁-C₈, C₁-C₆, or C₁-C₄), R^{37C}-substituted or unsubstituted heteroalkyl (e.g., 2 to 8 membered, 2 to 6 membered, or 2 to 4 membered), R^{37C}-substituted or unsubstituted cycloalkyl (e.g., C₃-C₈, C₃-C₆, or C₅-C₆), R^{37C}-substituted or unsubstituted heterocycloalkyl (e.g., 3 to 8 membered, 3 to 6 membered, or 5 to 6 membered), R^{37C}-substituted or unsubstituted aryl (e.g., C₆-C₁₀, C₁₀, or phenyl), or R^{37C}-substituted or unsubstituted heteroaryl (e.g., 5 to 10 membered, 5 to 9 membered, or 5 to 6 membered). X^{36C} is —F, —Cl, —Br, or —I.

In embodiments, R^{6D} is independently hydrogen, —CX^{6D}₃, —CN, —COOH, —CONH₂, —CHX^{6D}₂, —CH₂X^{6D}, R^{35D}-substituted or unsubstituted alkyl, R^{35D}-substituted or unsubstituted heteroalkyl, R^{35D}-substituted or unsubstituted cycloalkyl, R^{35D}-substituted or unsubstituted heterocycloalkyl, R^{35D}-substituted or unsubstituted aryl, or R^{35D}-substituted or unsubstituted heteroaryl. In embodiments, R^{6D} is independently hydrogen, —CX^{6D}₃, —CN, —COOH, —CONH₂, —CHX^{6D}₂, —CH₂X^{6D}, R^{35D}-substituted or unsubstituted C₁-C₈ alkyl, R^{35D}-substituted or unsubstituted 2 to 8 membered heteroalkyl, R^{35D}-substituted or unsubstituted C₃-C₈ cycloalkyl, R^{35D}-substituted or unsubstituted 3 to 6 membered heterocycloalkyl, R^{35D}-substituted or unsubstituted phenyl, or R^{35D}-substituted or unsubstituted 5 to 6 membered heteroaryl. X^{6D} is —F, —Cl, —Br, or —I. In embodiments, R^{6D} is independently hydrogen. In embodiments, R^{6D} is independently methyl. In embodiments, R^{6D} is independently ethyl.

R^{35D} is independently oxo, halogen, —CX^{35D}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{35D}₃, —OCHX^{35D}₂, R^{36D}-substituted or unsubstituted alkyl (e.g., C₁-C₈, C₁-C₆, or C₁-C₄), R^{36D}-substituted or unsubstituted heteroalkyl (e.g., 2 to 8 membered, 2 to 6 membered, or 2 to 4 membered), R^{36D}-substituted or unsubstituted cycloalkyl (e.g., C₃-C₈, C₃-C₆, or C₅-C₆), R^{36D}-substituted or unsubstituted heterocycloalkyl (e.g., 3 to 8 membered, 3 to 6 membered, or 5 to 6 membered), R^{36D}-substituted or unsubstituted aryl (e.g., C₆-C₁₀, C₁₀, or phenyl), or R^{36D}-substituted or unsubstituted heteroaryl (e.g., 5 to 10 membered, 5 to 9 membered, or 5 to 6 membered). X^{35D} is —F, —Cl, —Br, or —I.

R^{36D} is independently oxo, halogen, —CX^{36D}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{36D}₃, —OCHX^{36D}₂, R^{37D}-substituted or unsubstituted alkyl (e.g., C₁-C₈, C₁-C₆, or C₁-C₄), R^{37D}-substituted or unsubstituted heteroalkyl (e.g., 2 to 8 membered, 2 to 6 membered, or 2 to 4 membered), R^{37D}-substituted or unsubstituted cycloalkyl (e.g., C₃-C₈, C₃-C₆, or C₅-C₆), R^{37D}-substituted or unsubstituted heterocycloalkyl (e.g., 3 to 8 membered, 3 to 6 membered, or 5 to 6 membered), R^{37D}-substituted or unsubstituted aryl (e.g., C₆-C₁₀, C₁₀, or phenyl), or R^{37D}-substituted or unsubstituted heteroaryl (e.g., 5 to 10 membered, 5 to 9 membered, or 5 to 6 membered). X^{36D} is —F, —Cl, —Br, or —I.

R³⁷, R^{37A}, R^{37B}, R^{37C}, and R^{37D} are independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, L² is —NR⁷—, substituted or unsubstituted heteroalkylene, or substituted or unsubstituted heterocycloalkylene. In embodiments, L² is —NH—.

In embodiments, L² is —NR⁷— or substituted or unsubstituted heterocycloalkylene including a ring nitrogen bonded directly to E. In embodiments, L² is —NR⁷—. In embodiments, L² is substituted or unsubstituted heterocycloalkylene. In embodiments, L² is substituted or unsubstituted piperidinylene or substituted or unsubstituted pyrrolindinylene. In embodiments, L² is an unsubstituted piperidinylene or unsubstituted pyrrolindinylene.

In embodiments, L² is a bond. In embodiments, L² is —S(O)₂—. In embodiments, L² is —S(O)₂-Ph-. In embodiments, L² is —NR⁷—. In embodiments, L² is —O—. In embodiments, L² is —S—. In embodiments, L² is —C(O)—. In embodiments, L² is —C(O)NR⁷—. In embodiments, L² is —NR⁷C(O)—. In embodiments, L² is —NR⁷C(O)NH—. In embodiments, L² is —NHC(O)NR⁷—. In embodiments, L² is —C(O)O—. In embodiments, L² is —OC(O)—. In embodiments, L² is —NH—. In embodiments, L² is —C(O)NH—. In embodiments, L² is —NHC(O)—. In embodiments, L² is —NHC(O)NH—. In embodiments, L² is —CH₂—. In embodiments, L² is —OCH₂—. In embodiments, L² is —CH₂O—. In embodiments, L² is —CH₂CH₂—. In embodiments, L² is —SCH₂—. In embodiments, L² is —CH₂S—. In embodiments, L² is —CHCH—. In embodiments, L² is —CC—. In embodiments, L² is —NHCH₂—. In embodiments, L² is —CH₂NH—.

In embodiments, L² is a substituted or unsubstituted alkylene. In embodiments, L² is a substituted or unsubstituted heteroalkylene. In embodiments, L² is a substituted or unsubstituted cycloalkylene. In embodiments, L² is a substituted or unsubstituted heterocycloalkylene. In embodiments, L² is a substituted or unsubstituted arylene. In embodiments, L² is a substituted or unsubstituted heteroarylene. In embodiments, L² is a substituted alkylene. In embodiments, L² is a substituted heteroalkylene. In embodiments, L² is a substituted cycloalkylene. In embodiments, L² is a substituted heterocycloalkylene. In embodiments, L² is a substituted arylene. In embodiments, L² is a substituted heteroarylene. In embodiments, L² is an unsubstituted alkylene. In embodiments, L² is an unsubstituted heteroalkylene. In embodiments, L² is an unsubstituted cycloalkylene. In embodiments, L² is an unsubstituted heterocycloalkylene. In embodiments, L² is an unsubstituted arylene. In embodiments, L² is an unsubstituted heteroarylene. In embodiments, L² is a substituted or unsubstituted C₁-C₈ alkylene. In embodiments, L² is a substituted or unsubstituted 2 to 8 membered heteroalkylene. In embodiments, L² is a substituted or unsubstituted C₃-C₈ cycloalkylene. In embodiments, L² is a substituted or unsubstituted 3 to 8 membered heterocycloalkylene. In embodiments, L² is a substituted or unsubstituted C₆-C₁₀ arylene. In embodiments, L² is a substituted or unsubstituted 5 to 10 membered heteroarylene.

In embodiments, L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, R⁴⁴-substituted or unsubstituted alkylene, R⁴⁴-substituted or unsubstituted heteroalkylene, R⁴⁴-substituted or unsubstituted cycloalkylene, R⁴⁴-substituted or unsubstituted heterocycloalkylene, R⁴⁴-substituted or unsubstituted arylene, or R⁴⁴-substituted or unsubstituted heteroarylene. In embodiments, L² is a bond, —S(O)₂—, —S(O)₂-Ph-, —NH—, —O—, —S—, —C(O)—, —C(O)NH—, —NHC(O)—, —NHC(O)NH—, —C(O)O—, —OC(O)—, R⁴⁴-substituted or unsubstituted alkylene, R⁴⁴-substituted or unsubstituted heteroalkylene, R⁴⁴-substituted or unsubstituted cycloalkylene, R⁴⁴-substituted or unsubstituted heterocycloalkylene, R⁴⁴-substituted or unsubstituted arylene, or R⁴⁴-substituted or unsubstituted heteroarylene. In embodiments, L² is a bond, —S(O)₂—, —S(O)₂-Ph-, —NH—, —O—, —S—, —C(O)—, —C(O)NH—, —NHC(O)—, —NHC(O)NH—, —C(O)O—, —OC(O)—, R⁴⁴-substituted or unsubstituted C₁-C₈ alkylene, R⁴⁴-substituted or unsubstituted 2 to 8 membered heteroalkylene, R⁴⁴-substituted or unsubstituted C₃-C₈ cycloalkylene, R⁴⁴-substituted or unsubstituted 3 to 6 membered heterocycloalkylene, R⁴⁴-substituted or unsubstituted phenylene, or R⁴⁴-substituted or unsubstituted 5 to 6 membered heteroarylene.

R⁴⁴ is independently oxo, halogen, —CX⁴⁴₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX⁴⁴₃, —OCHX⁴⁴₂, R⁴⁵-substituted or unsubstituted alkyl (e.g., C₁-C₈, C₁-C₆, or C₁-C₄), R⁴⁵-substituted or unsubstituted heteroalkyl (e.g., 2 to 8 membered, 2 to 6 membered, or 2 to 4 membered), R⁴⁵-substituted or unsubstituted cycloalkyl (e.g., C₃-C₈, C₃-C₆, or C₅-C₆), R⁴⁵-substituted or unsubstituted heterocycloalkyl (e.g., 3 to 8 membered, 3 to 6 membered, or 5 to 6 membered), R⁴⁵-substituted or unsubstituted aryl (e.g., C₆-C₁₀, C₁₀, or phenyl), or R⁴⁵-substituted or unsubstituted heteroaryl (e.g., 5 to 10 membered, 5 to 9 membered, or 5 to 6 membered). X⁴⁴ is —F, —Cl, —Br, or —I.

R⁴⁵ is independently oxo, halogen, —CX⁴⁵₃, —CHX⁴⁵₂, —CH₂X⁴⁵, —OCX⁴⁵₃, —OCHX⁴⁵₂, —OCH₂X⁴⁵, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁴⁶-substituted or unsubstituted alkyl (e.g., C₁-C₈, C₁-C₆, or C₁-C₄), R⁴⁶-substituted or unsubstituted heteroalkyl (e.g., 2 to 8 membered, 2 to 6 membered, or 2 to 4 membered), R⁴⁶-substituted or unsubstituted cycloalkyl (e.g., C₃-C₈, C₃-C₆, or C₅-C₆), R⁴⁶-substituted or unsubstituted heterocycloalkyl (e.g., 3 to 8 membered, 3 to 6 membered, or 5 to 6 membered), R⁴⁶-substituted or unsubstituted aryl (e.g., C₆-C₁₀, C₁₀, or phenyl), or R⁴⁶-substituted or unsubstituted heteroaryl (e.g., 5 to 10 membered, 5 to 9 membered, or 5 to 6 membered). X⁴⁵ is —F, —Cl, —Br, or —I.

R⁴⁶ is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, L² is R⁴⁴-substituted or unsubstituted 4 membered heterocycloalkylene. In embodiments, L² is R⁴⁴-substituted or unsubstituted 5 membered heterocycloalkylene. In embodiments, L² is R⁴⁴-substituted or unsubstituted 6 membered heterocycloalkylene. In embodiments, L² is R⁴⁴-substituted or unsubstituted 7 membered heterocycloalkylene. In embodiments, L² is R⁴⁴-substituted or unsubstituted heterocycloalkylene (e.g., 3 to 6 membered heterocycloalkylene, 4 to 6 membered heterocycloalkylene, or 5 to 6 membered heterocycloalkylene). In embodiments, L² is R⁴⁴-substituted 4 membered heterocycloalkylene. In embodiments, L² is R⁴⁴-substituted 5 membered heterocycloalkylene. In embodiments, L² is R⁴⁴-substituted 6 membered heterocycloalkylene. In embodiments, L² is R⁴⁴-substituted 7 membered heterocycloalkylene. In embodiments, L² is R⁴⁴-substituted heterocycloalkylene (e.g., 3 to 6 membered heterocycloalkylene, 4 to 6 membered heterocycloalkylene, or 5 to 6 membered heterocycloalkylene). In embodiments, L² is an unsubstituted 4 membered heterocycloalkylene. In embodiments, L² is an unsubstituted 5 membered heterocycloalkylene. In embodiments, L² is an unsubstituted 6 membered heterocycloalkylene. In embodiments, L² is an unsubstituted 7 membered heterocycloalkylene. In embodiments, L² is an unsubstituted heterocycloalkylene (e.g., 3 to 6 membered heterocycloalkylene, 4 to 6 membered heterocycloalkylene, or 5 to 6 membered heterocycloalkylene).

In embodiments, L² is R⁴⁴-substituted or unsubstituted piperidinylene. In embodiments, L² is R⁴⁴-substituted or unsubstituted pyrrolidinylene. In embodiments, L² is R⁴⁴-substituted or unsubstituted imidazolidinylene. In embodiments, L² is R⁴⁴-substituted or unsubstituted pyrazolidinylene. In embodiments, L² is R⁴⁴-substituted or unsubstituted piperazinylene. In embodiments, L² is R⁴⁴-substituted or unsubstituted piperazinylene. In embodiments, L² is R⁴⁴-substituted or unsubstituted azetidinylene. In embodiments, L² is R⁴⁴-substituted or unsubstituted aziridinylene. In embodiments, L² is R⁴⁴-substituted or unsubstituted morpholinylene.

In embodiments, L² is a R⁴⁴-substituted or unsubstituted 5 to 10 membered heteroarylene. In embodiments, L² is a R⁴⁴-substituted or unsubstituted 5 to 6 membered heteroarylene. In embodiments, L² is a R⁴⁴-substituted or unsubstituted pyridinylene, pyridazinylene, pyrimidinylene, pyrazinylene, or triazinylene.

In embodiments, L² is R⁴⁴-substituted or unsubstituted indolinylene. In embodiments, L² is R⁴⁴-substituted or unsubstituted indazolylene. In embodiments, L² is R⁴⁴-substituted or unsubstituted benzimidazolylene. In embodiments, L² is R⁴⁴-substituted or unsubstituted benzoxazolylene. In embodiments, L² is R⁴⁴-substituted or unsubstituted azaindolylene. In embodiments, L² is R⁴⁴-substituted or unsubstituted purinylene. In embodiments, L² is R⁴⁴-substituted or unsubstituted indolylene. In embodiments, L² is R⁴⁴-substituted or unsubstituted pyrazinylene. In embodiments, L² is R⁴⁴-substituted or unsubstituted pyrrolylene. In embodiments, L² is R⁴⁴-substituted or unsubstituted imidazolylene. In embodiments, L² is R⁴⁴-substituted or unsubstituted pyrazolylene. In embodiments, L² is R⁴⁴-substituted or unsubstituted triazolylene. In embodiments, L² is R⁴⁴-substituted or unsubstituted tetrazolylene. In embodiments, L² is R⁴⁴-substituted or unsubstituted azepanylene. In embodiments, L² is R⁴⁴-substituted or unsubstituted azepinylene.

In embodiments, L² is R⁴⁴-substituted or unsubstituted C₃-C₈ cycloalkylene. In embodiments, L² is R⁴⁴-substituted or unsubstituted C₄-C₆ cycloalkylene. In embodiments, L² is R⁴⁴-substituted or unsubstituted C₅-C₆ cycloalkylene. In embodiments, L² is R⁴⁴-substituted or unsubstituted cycloalkylene (e.g., C₃-C₈ cycloalkylene, C₄-C₆ cycloalkylene, or C₅-C₆ cycloalkylene). In embodiments, L² is R⁴⁴-substituted C₃-C₈ cycloalkylene. In embodiments, L² is R⁴⁴-substituted C₄-C₆ cycloalkylene. In embodiments, L² is R⁴⁴-substituted C₅-C₆ cycloalkylene. In embodiments, L² is R⁴⁴-substituted cyclocalkylene (e.g., C₃-C₈ cycloalkylene, C₄-C₆ cycloalkylene, or C₅-C₆ cycloalkylene). In embodiments, L² is an unsubstituted C₃-C₈ cycloalkylene. In embodiments, L² is an unsubstituted C₄-C₆ cycloalkylene. In embodiments, L² is an unsubstituted C₅-C₆ cycloalkylene. In embodiments, L² is an unsubstituted cycloalkylene (e.g., C₃-C₈ cycloalkylene, C₄-C₆ cycloalkylene, or C₅-C₆ cycloalkylene). In embodiments, L² is R⁴⁴-substituted or unsubstituted arylene (e.g., C₆-C₁₀, C₁₀, or phenyl). In embodiments, L² is R⁴⁴-substituted arylene (e.g., C₆-C₁₀, C₁₀, or phenyl). In embodiments, L² is an unsubstituted arylene (e.g., C₆-C₁₀, C₁₀, or phenyl).

In embodiments, L² is R⁴⁴-substituted or unsubstituted alkylene (e.g., C₁-C₈ alkylene, C₁-C₆ alkylene, C₁-C₄ alkylene, C₁-C₂ alkylene). In embodiments, L² is R⁴⁴-substituted or unsubstituted C₁-C₈ alkylene. In embodiments, L² is R⁴⁴-substituted or unsubstituted C₁-C₆ alkylene. In embodiments, L² is R⁴⁴-substituted or unsubstituted C₁-C₄ alkylene. In embodiments, L² is R⁴⁴-substituted or unsubstituted C₁-C₂ alkylene. In embodiments, L² is R⁴⁴-substituted alkylene (e.g., C₁-C₈ alkylene, C₁-C₆ alkylene, C₁-C₄ alkylene, C₁-C₂ alkylene). In embodiments, L² is R⁴⁴-substituted C₁-C₈ alkylene. In embodiments, L² is R⁴⁴-substituted C₁-C₆ alkylene. In embodiments, L² is R⁴⁴-substituted C₁-C₄ alkylene. In embodiments, L² is R⁴⁴-substituted C₁-C₂ alkylene. In embodiments, L² is an unsubstituted alkylene (e.g., C₁-C₈ alkylene, C₁-C₆ alkylene, C₁-C₄ alkylene, C₁-C₂ alkylene). In embodiments, L² is an unsubstituted C₁-C₈ alkylene. In embodiments, L² is an unsubstituted C₁-C₆ alkylene. In embodiments, L² is an unsubstituted C₁-C₄ alkylene. In embodiments, L² is an unsubstituted C₁-C₂ alkylene. In embodiments, L² is R⁴⁴-substituted or unsubstituted methylene. In embodiments, L² is an unsubstituted methylene.

In embodiments, L² is R⁴⁴-substituted or unsubstituted heteroalkylene (e.g., 2 to 8 membered heteroalkylene, 2 to 6 membered heteroalkylene, 2 to 4 membered heteroalkylene). In embodiments, L² is R⁴⁴-substituted or unsubstituted 2 to 8 membered heteroalkylene. In embodiments, L² is R⁴⁴-substituted or unsubstituted 2 to 6 membered heteroalkylene. In embodiments, L² is R⁴⁴-substituted or unsubstituted 2 to 4 membered heteroalkylene. In embodiments, L² is R⁴⁴-substituted heteroalkylene (e.g., 2 to 8 membered heteroalkylene, 2 to 6 membered heteroalkylene, 2 to 4 membered heteroalkylene). In embodiments, L² is R⁴⁴-substituted 2 to 8 membered heteroalkylene. In embodiments, L² is R⁴⁴-substituted 2 to 6 membered heteroalkylene. In embodiments, L² is R⁴⁴-substituted 2 to 4 membered heteroalkylene. In embodiments, L² is an unsubstituted heteroalkylene (e.g., 2 to 8 membered heteroalkylene, 2 to 6 membered heteroalkylene, 2 to 4 membered heteroalkylene). In embodiments, L² is an unsubstituted 2 to 8 membered heteroalkylene. In embodiments, L² is an unsubstituted 2 to 6 membered heteroalkylene. In embodiments, L² is an unsubstituted 2 to 4 membered heteroalkylene.

In embodiments, R⁷ is hydrogen, substituted or unsubstituted C₁-C₆ alkyl, or substituted or unsubstituted 2 to 6 membered heteroalkyl. In embodiments, R⁷ is hydrogen or unsubstituted C₁-C₃ alkyl. In embodiments, R⁷ is hydrogen.

In embodiments, R⁷ is independently hydrogen. In embodiments, R⁷ is independently halogen. In embodiments, R⁷ is independently —CX⁷₃. In embodiments, R⁷ is independently —CHX⁷₂. In embodiments, R⁷ is independently —CH₂X⁷. In embodiments, R⁷ is independently —OCX⁷₃. In embodiments, R⁷ is independently —OCH₂X⁷. In embodiments, R⁷ is independently —OCHX⁷₂. In embodiments, R⁷ is independently —CN. In embodiments, R⁷ is independently —SOₙ₇R^{7D}. In embodiments, R⁷ is independently —SOᵥ₇NR^{7A}R^{7B}. In embodiments, R⁷ is independently —NHC(O)NR^{7A}R^{7B}. In embodiments, R⁷ is independently —N(O)ₘ₇. In embodiments, R⁷ is independently —NR^{7A}R^{7B}. In embodiments, R⁷ is independently —C(O)R^{7C}. In embodiments, R⁷ is independently —C(O)—OR^{7C}. In embodiments, R⁷ is independently —C(O)NR^{7A}R^{7B}. In embodiments, R⁷ is independently —OR^{7D}. In embodiments, R⁷ is independently —NR^{7A}SO₂R^{7D}. In embodiments, R⁷ is independently —NR^{7A}C(O)R^{7C}. In embodiments, R⁷ is independently —NR^{7A}C(O)OR^{7C}. In embodiments, R⁷ is independently —NR^{7A}OR^{7C}. In embodiments, R⁷ is independently —OH. In embodiments, R⁷ is independently —NH₂. In embodiments, R⁷ is independently —COOH. In embodiments, R⁷ is independently —CONH₂. In embodiments, R⁷ is independently —NO₂. In embodiments, R⁷ is independently —SH.

In embodiments, R⁷ is independently substituted or unsubstituted alkyl (e.g., C₁-C₈, C₁-C₆, or C₁-C₄). In embodiments, R⁷ is independently substituted alkyl (e.g., C₁-C₈, C₁-C₆, or C₁-C₄). In embodiments, R⁷ is independently unsubstituted alkyl (e.g., C₁-C₈, C₁-C₆, or C₁-C₄). In embodiments, R⁷ is independently substituted or unsubstituted heteroalkyl (e.g., 2 to 8 membered, 2 to 6 membered, or 2 to 4 membered). In embodiments, R⁷ is independently substituted heteroalkyl (e.g., 2 to 8 membered, 2 to 6 membered, or 2 to 4 membered). In embodiments, R⁷ is independently unsubstituted heteroalkyl (e.g., 2 to 8 membered, 2 to 6 membered, or 2 to 4 membered). In embodiments, R⁷ is independently substituted or unsubstituted cycloalkyl (e.g., C₃-C₈, C₃-C₆, or C₅-C₆). In embodiments, R⁷ is independently substituted cycloalkyl (e.g., C₃-C₈, C₃-C₆, or C₅-C₆). In embodiments, R⁷ is independently unsubstituted cycloalkyl (e.g., C₃-C₈, C₃-C₆, or C₅-C₆). In embodiments, R⁷ is independently substituted or unsubstituted heterocycloalkyl (e.g., 3 to 8 membered, 3 to 6 membered, or 5 to 6 membered). In embodiments, R⁷ is independently substituted heterocycloalkyl (e.g., 3 to 8 membered, 3 to 6 membered, or 5 to 6 membered). In embodiments, R⁷ is independently unsubstituted heterocycloalkyl (e.g., 3 to 8 membered, 3 to 6 membered, or 5 to 6 membered). In embodiments, R⁷ is independently substituted or unsubstituted aryl (e.g., C₆-C₁₀, C₁₀, or phenyl). In embodiments, R⁷ is independently substituted aryl (e.g., C₆-C₁₀, C₁₀, or phenyl). In embodiments, R⁷ is independently unsubstituted aryl (e.g., C₆-C₁₀, C₁₀, or phenyl). In embodiments, R⁷ is independently substituted or unsubstituted heteroaryl (e.g., 5 to 10 membered, 5 to 9 membered, or 5 to 6 membered). In embodiments, R⁷ is independently substituted heteroaryl (e.g., 5 to 10 membered, 5 to 9 membered, or 5 to 6 membered). In embodiments, R⁷ is independently unsubstituted heteroaryl (e.g., 5 to 10 membered, 5 to 9 membered, or 5 to 6 membered).

In embodiments, R^{7A} is independently hydrogen. In embodiments, R^{7A} is independently —CX^{7A}₃. In embodiments, R^{7A} is independently —CHX^{7A}₂. In embodiments, R^{7A} is independently —CH₂X^{7A}. In embodiments, R^{7A} is independently —CN. In embodiments, R^{7A} is independently —COOH. In embodiments, R^{7A} is independently —CONH₂.

In embodiments, R^{7A} is independently substituted or unsubstituted alkyl (e.g., C₁-C₈, C₁-C₆, or C₁-C₄). In embodiments, R^{7A} is independently substituted alkyl (e.g., C₁-C₈, C₁-C₆, or C₁-C₄). In embodiments, R^{7A} is independently unsubstituted alkyl (e.g., C₁-C₈, C₁-C₆, or C₁-C₄). In embodiments, R^{7A} is independently substituted or unsubstituted heteroalkyl (e.g., 2 to 8 membered, 2 to 6 membered, or 2 to 4 membered). In embodiments, R^{7A} is independently substituted heteroalkyl (e.g., 2 to 8 membered, 2 to 6 membered, or 2 to 4 membered). In embodiments, R^{7A} is independently unsubstituted heteroalkyl (e.g., 2 to 8 membered, 2 to 6 membered, or 2 to 4 membered). In embodiments, R^{7A} is independently substituted or unsubstituted cycloalkyl (e.g., C₃-C₈, C₃-C₆, or C₅-C₆). In embodiments, R^{7A} is independently substituted cycloalkyl (e.g., C₃-C₈, C₃-C₆, or C₅-C₆). In embodiments, R^{7A} is independently unsubstituted cycloalkyl (e.g., C₃-C₈, C₃-C₆, or C₅-C₆). In embodiments, R^{7A} is independently substituted or unsubstituted heterocycloalkyl (e.g., 3 to 8 membered, 3 to 6 membered, or 5 to 6 membered). In embodiments, R^{7A} is independently substituted heterocycloalkyl (e.g., 3 to 8 membered, 3 to 6 membered, or 5 to 6 membered). In embodiments, R^{7A} is independently unsubstituted heterocycloalkyl (e.g., 3 to 8 membered, 3 to 6 membered, or 5 to 6 membered). In embodiments, R^{7A} is independently substituted or unsubstituted aryl (e.g., C₆-C₁₀, C₁₀, or phenyl). In embodiments, R^{7A} is independently substituted aryl (e.g., C₆-C₁₀, C₁₀, or phenyl). In embodiments, R^{7A} is independently unsubstituted aryl (e.g., C₆-C₁₀, C₁₀, or phenyl). In embodiments, R^{7A} is independently substituted or unsubstituted heteroaryl (e.g., 5 to 10 membered, 5 to 9 membered, or 5 to 6 membered). In embodiments, R^{7A} is independently substituted heteroaryl (e.g., 5 to 10 membered, 5 to 9 membered, or 5 to 6 membered). In embodiments, R^{7A} is independently unsubstituted heteroaryl (e.g., 5 to 10 membered, 5 to 9 membered, or 5 to 6 membered). In embodiments, R^{7A} is independently unsubstituted methyl. In embodiments, R^{7A} is independently unsubstituted ethyl. In embodiments, R^{7A} is independently unsubstituted propyl. In embodiments, R^{7A} is independently unsubstituted isopropyl. In embodiments, R^{7A} is independently unsubstituted tert-butyl.

In embodiments, R^{7B} is independently hydrogen. In embodiments, R^{7B} is independently —CX^{7B}₃. In embodiments, R^{7B} is independently —CHX^{7B}₂. In embodiments, R^{7B} is independently —CH₂X^{7B}. In embodiments, R^{7B} is independently —CN. In embodiments, R^{7B} is independently —COOH. In embodiments, R^{7B} is independently —CONH₂.

In embodiments, R^{7B} is independently substituted or unsubstituted alkyl (e.g., C₁-C₈, C₁-C₆, or C₁-C₄). In embodiments, R^{7B} is independently substituted alkyl (e.g., C₁-C₈, C₁-C₆, or C₁-C₄). In embodiments, R^{7B} is independently unsubstituted alkyl (e.g., C₁-C₈, C₁-C₆, or C₁-C₄). In embodiments, R^{7B} is independently substituted or unsubstituted heteroalkyl (e.g., 2 to 8 membered, 2 to 6 membered, or 2 to 4 membered). In embodiments, R^{7B} is independently substituted heteroalkyl (e.g., 2 to 8 membered, 2 to 6 membered, or 2 to 4 membered). In embodiments, R^{7B} is independently unsubstituted heteroalkyl (e.g., 2 to 8 membered, 2 to 6 membered, or 2 to 4 membered). In embodiments, R^{7B} is independently substituted or unsubstituted cycloalkyl (e.g., C₃-C₈, C₃-C₆, or C₅-C₆). In embodiments, R^{7B} is independently substituted cycloalkyl (e.g., C₃-C₈, C₃-C₆, or C₅-C₆). In embodiments, R^{7B} is independently unsubstituted cycloalkyl (e.g., C₃-C₈, C₃-C₆, or C₅-C₆). In embodiments, R^{7B} is independently substituted or unsubstituted heterocycloalkyl (e.g., 3 to 8 membered, 3 to 6 membered, or 5 to 6 membered). In embodiments, R^{7B} is independently substituted heterocycloalkyl (e.g., 3 to 8 membered, 3 to 6 membered, or 5 to 6 membered). In embodiments, R^{7B} is independently unsubstituted heterocycloalkyl (e.g., 3 to 8 membered, 3 to 6 membered, or 5 to 6 membered). In embodiments, R^{7B} is independently substituted or unsubstituted aryl (e.g., C₆-C₁₀, C₁₀, or phenyl). In embodiments, R^{7B} is independently substituted aryl (e.g., C₆-C₁₀, C₁₀, or phenyl). In embodiments, R^{7B} is independently unsubstituted aryl (e.g., C₆-C₁₀, C₁₀, or phenyl). In embodiments, R^{7B} is independently substituted or unsubstituted heteroaryl (e.g., 5 to 10 membered, 5 to 9 membered, or 5 to 6 membered). In embodiments, R^{7B} is independently substituted heteroaryl (e.g., 5 to 10 membered, 5 to 9 membered, or 5 to 6 membered). In embodiments, R^{7B} is independently unsubstituted heteroaryl (e.g., 5 to 10 membered, 5 to 9 membered, or 5 to 6 membered). In embodiments, R^{7B} is independently unsubstituted methyl. In embodiments, R^{7B} is independently unsubstituted ethyl. In embodiments, R^{7B} is independently unsubstituted propyl. In embodiments, R^{7B} is independently unsubstituted isopropyl. In embodiments, R^{7B} is independently unsubstituted tert-butyl.

In embodiments, R^{7A} and R^{7B} substituents bonded to the same nitrogen atom may be joined to form a substituted or unsubstituted heterocycloalkyl. In embodiments, R^{7A} and R^{7B} substituents bonded to the same nitrogen atom may be joined to form a substituted or unsubstituted heteroaryl. In embodiments, R^{7A} and R^{7B} substituents bonded to the same nitrogen atom may be joined to form a substituted heterocycloalkyl. In embodiments, R^{7A} and R^{7B} substituents bonded to the same nitrogen atom may be joined to form a substituted heteroaryl. In embodiments, R^{7A} and R^{7B} substituents bonded to the same nitrogen atom may be joined to form an unsubstituted heterocycloalkyl. In embodiments, R^{7A} and R^{7B} substituents bonded to the same nitrogen atom may be joined to form an unsubstituted heteroaryl. In embodiments, R^{7A} and R^{7B} substituents bonded to the same nitrogen atom may be joined to form a substituted or unsubstituted 3 to 8 membered heterocycloalkyl. In embodiments, R^{7A} and R^{7B} substituents bonded to the same nitrogen atom may be joined to form a substituted or unsubstituted 5 to 10 membered heteroaryl. In embodiments, R^{7A} and R^{7B} substituents bonded to the same nitrogen atom may be joined to form a substituted 3 to 8 membered heterocycloalkyl. In embodiments, R^{7A} and R^{7B} substituents bonded to the same nitrogen atom may be joined to form a substituted 5 to 10 membered heteroaryl. In embodiments, R^{7A} and R^{7B} substituents bonded to the same nitrogen atom may be joined to form an unsubstituted 3 to 8 membered heterocycloalkyl. In embodiments, R^{7A} and R^{7B} substituents bonded to the same nitrogen atom may be joined to form an unsubstituted 5 to 10 membered heteroaryl. In embodiments, R^{7A} and R^{7B} substituents bonded to the same nitrogen atom may be joined to form a substituted or unsubstituted 3 to 6 membered heterocycloalkyl. In embodiments, R^{7A} and R^{7B} substituents bonded to the same nitrogen atom may be joined to form a substituted or unsubstituted 5 to 6 membered heteroaryl. In embodiments, R^{7A} and R^{7B} substituents bonded to the same nitrogen atom may be joined to form a substituted 3 to 6 membered heterocycloalkyl. In embodiments, R^{7A} and R^{7B} substituents bonded to the same nitrogen atom may be joined to form a substituted 5 to 6 membered heteroaryl. In embodiments, R^{7A} and R^{7B} substituents bonded to the same nitrogen atom may be joined to form an unsubstituted 3 to 6 membered heterocycloalkyl. In embodiments, R^{7A} and R^{7B} substituents bonded to the same nitrogen atom may be joined to form an unsubstituted 5 to 6 membered heteroaryl.

In embodiments, R^{7C} is independently hydrogen. In embodiments, R^{7C} is independently —CX^{7C}₃. In embodiments, R^{7C} is independently —CHX^{7C}₂. In embodiments, R^{7C} is independently —CH₂X^{7C}. In embodiments, R^{7C} is independently —CN. In embodiments, R^{7C} is independently —COOH. In embodiments, R^{7C} is independently —CONH₂.

In embodiments, R^{7C} is independently substituted or unsubstituted alkyl (e.g., C₁-C₈, C₁-C₆, or C₁-C₄). In embodiments, R^{7C} is independently substituted alkyl (e.g., C₁-C₈, C₁-C₆, or C₁-C₄). In embodiments, R^{7C} is independently unsubstituted alkyl (e.g., C₁-C₈, C₁-C₆, or C₁-C₄). In embodiments, R^{7C} is independently substituted or unsubstituted heteroalkyl (e.g., 2 to 8 membered, 2 to 6 membered, or 2 to 4 membered). In embodiments, R^{7C} is independently substituted heteroalkyl (e.g., 2 to 8 membered, 2 to 6 membered, or 2 to 4 membered). In embodiments, R^{7C} is independently unsubstituted heteroalkyl (e.g., 2 to 8 membered, 2 to 6 membered, or 2 to 4 membered). In embodiments, R^{7C} is independently substituted or unsubstituted cycloalkyl (e.g., C₃-C₈, C₃-C₆, or C₅-C₆). In embodiments, R^{7C} is independently substituted cycloalkyl (e.g., C₃-C₈, C₃-C₆, or C₅-C₆). In embodiments, R^{7C} is independently unsubstituted cycloalkyl (e.g., C₃-C₈, C₃-C₆, or C₅-C₆). In embodiments, R^{7C} is independently substituted or unsubstituted heterocycloalkyl (e.g., 3 to 8 membered, 3 to 6 membered, or 5 to 6 membered). In embodiments, R^{7C} is independently substituted heterocycloalkyl (e.g., 3 to 8 membered, 3 to 6 membered, or 5 to 6 membered). In embodiments, R^{7C} is independently unsubstituted heterocycloalkyl (e.g., 3 to 8 membered, 3 to 6 membered, or 5 to 6 membered). In embodiments, R^{7C} is independently substituted or unsubstituted aryl (e.g., C₆-C₁₀, C₁₀, or phenyl). In embodiments, R^{7C} is independently substituted aryl (e.g., C₆-C₁₀, C₁₀, or phenyl). In embodiments, R^{7C} is independently unsubstituted aryl (e.g., C₆-C₁₀, C₁₀, or phenyl). In embodiments, R^{7C} is independently substituted or unsubstituted heteroaryl (e.g., 5 to 10 membered, 5 to 9 membered, or 5 to 6 membered). In embodiments, R^{7C} is independently substituted heteroaryl (e.g., 5 to 10 membered, 5 to 9 membered, or 5 to 6 membered). In embodiments, R^{7C} is independently unsubstituted heteroaryl (e.g., 5 to 10 membered, 5 to 9 membered, or 5 to 6 membered). In embodiments, R^{7C} is independently unsubstituted methyl. In embodiments, R^{7C} is independently unsubstituted ethyl. In embodiments, R^{7C} is independently unsubstituted propyl. In embodiments, R^{7C} is independently unsubstituted isopropyl. In embodiments, R^{7C} is independently unsubstituted tert-butyl.

In embodiments, R^{7D} is independently hydrogen. In embodiments, R^{7D} is independently —CX^{7D}₃. In embodiments, R^{7D} is independently —CHX^{7D}₂. In embodiments, R^{7D} is independently —CH₂X^{7D}. In embodiments, R^{7D} is independently —CN. In embodiments, R^{7D} is independently —COOH. In embodiments, R^{7D} is independently —CONH₂.

In embodiments, R^{7D} is independently substituted or unsubstituted alkyl (e.g., C₁-C₈, C₁-C₆, or C₁-C₄). In embodiments, R^{7D} is independently substituted alkyl (e.g., C₁-C₈, C₁-C₆, or C₁-C₄). In embodiments, R^{7D} is independently unsubstituted alkyl (e.g., C₁-C₈, C₁-C₆, or C₁-C₄). In embodiments, R^{7D} is independently substituted or unsubstituted heteroalkyl (e.g., 2 to 8 membered, 2 to 6 membered, or 2 to 4 membered). In embodiments, R^{7D} is independently substituted heteroalkyl (e.g., 2 to 8 membered, 2 to 6 membered, or 2 to 4 membered). In embodiments, R^{7D} is independently unsubstituted heteroalkyl (e.g., 2 to 8 membered, 2 to 6 membered, or 2 to 4 membered). In embodiments, R^{7D} is independently substituted or unsubstituted cycloalkyl (e.g., C₃-C₈, C₃-C₆, or C₅-C₆). In embodiments, R^{7D} is independently substituted cycloalkyl (e.g., C₃-C₈, C₃-C₆, or C₅-C₆). In embodiments, R^{7D} is independently unsubstituted cycloalkyl (e.g., C₃-C₈, C₃-C₆, or C₅-C₆). In embodiments, R^{7D} is independently substituted or unsubstituted heterocycloalkyl (e.g., 3 to 8 membered, 3 to 6 membered, or 5 to 6 membered). In embodiments, R^{7D} is independently substituted heterocycloalkyl (e.g., 3 to 8 membered, 3 to 6 membered, or 5 to 6 membered). In embodiments, R^{7D} is independently unsubstituted heterocycloalkyl (e.g., 3 to 8 membered, 3 to 6 membered, or 5 to 6 membered). In embodiments, R^{7D} is independently substituted or unsubstituted aryl (e.g., C₆-C₁₀, C₁₀, or phenyl). In embodiments, R^{7D} is independently substituted aryl (e.g., C₆-C₁₀, C₁₀, or phenyl). In embodiments, R^{7D} is independently unsubstituted aryl (e.g., C₆-C₁₀, C₁₀, or phenyl). In embodiments, R^{7D} is independently substituted or unsubstituted heteroaryl (e.g., 5 to 10 membered, 5 to 9 membered, or 5 to 6 membered). In embodiments, R^{7D} is independently substituted heteroaryl (e.g., 5 to 10 membered, 5 to 9 membered, or 5 to 6 membered). In embodiments, R^{7D} is independently unsubstituted heteroaryl (e.g., 5 to 10 membered, 5 to 9 membered, or 5 to 6 membered). In embodiments, R^{7D} is independently unsubstituted methyl. In embodiments, R^{7D} is independently unsubstituted ethyl. In embodiments, R^{7D} is independently unsubstituted propyl. In embodiments, R^{7D} is independently unsubstituted isopropyl. In embodiments, R^{7D} is independently unsubstituted tert-butyl.

In embodiments, R⁷ is independently hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, R³⁸-substituted or unsubstituted alkyl, R³⁸-substituted or unsubstituted heteroalkyl, R³⁸-substituted or unsubstituted cycloalkyl, R³⁸-substituted or unsubstituted heterocycloalkyl, R³⁸-substituted or unsubstituted aryl, or R³⁸-substituted or unsubstituted heteroaryl. In embodiments, R⁷ is independently halogen, —CX⁷₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX⁷₃, —OCHX⁷₂, R³⁸-substituted or unsubstituted alkyl, R³⁸-substituted or unsubstituted heteroalkyl, R³⁸-substituted or unsubstituted cycloalkyl, R³⁸-substituted or unsubstituted heterocycloalkyl, R³⁸-substituted or unsubstituted aryl, or R³⁸-substituted or unsubstituted heteroaryl. In embodiments, R⁷ is independently halogen, —CX⁷₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX⁷₃, —OCHX⁷₂, R³⁸-substituted or unsubstituted C₁-C₈ alkyl, R³⁸-substituted or unsubstituted 2 to 8 membered heteroalkyl, R³⁸-substituted or unsubstituted C₃-C₈ cycloalkyl, R³⁸-substituted or unsubstituted 3 to 6 membered heterocycloalkyl, R³⁸-substituted or unsubstituted phenyl, or R³⁸-substituted or unsubstituted 5 to 6 membered heteroaryl. X⁷ is —F, —Cl, —Br, or —I. In embodiments, R⁷ is independently hydrogen. In embodiments, R⁷ is independently methyl. In embodiments, R⁷ is independently ethyl.

R³⁸ is independently oxo, halogen, —CX³⁸₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX³⁸₃, —OCHX³⁸₂, R³⁹-substituted or unsubstituted alkyl (e.g., C₁-C₈, C₁-C₆, or C₁-C₄), R³⁹-substituted or unsubstituted heteroalkyl (e.g., 2 to 8 membered, 2 to 6 membered, or 2 to 4 membered), R³⁹-substituted or unsubstituted cycloalkyl (e.g., C₃-C₈, C₃-C₆, or C₅-C₆), R³⁹-substituted or unsubstituted heterocycloalkyl (e.g., 3 to 8 membered, 3 to 6 membered, or 5 to 6 membered), R³⁹-substituted or unsubstituted aryl (e.g., C₆-C₁₀, C₁₀, or phenyl), or R³⁹-substituted or unsubstituted heteroaryl (e.g., 5 to 10 membered, 5 to 9 membered, or 5 to 6 membered). X³⁸ is —F, —Cl, —Br, or —I.

R³⁹ is independently oxo, halogen, —CX³⁹₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX³⁹₃, —OCHX³⁹₂, R⁴⁰-substituted or unsubstituted alkyl (e.g., C₁-C₈, C₁-C₆, or C₁-C₄), R⁴⁰-substituted or unsubstituted heteroalkyl (e.g., 2 to 8 membered, 2 to 6 membered, or 2 to 4 membered), R⁴⁰-substituted or unsubstituted cycloalkyl (e.g., C₃-C₈, C₃-C₆, or C₅-C₆), R⁴⁰-substituted or unsubstituted heterocycloalkyl (e.g., 3 to 8 membered, 3 to 6 membered, or 5 to 6 membered), R⁴⁰-substituted or unsubstituted aryl (e.g., C₆-C₁₀, C₁₀, or phenyl), or R⁴⁰-substituted or unsubstituted heteroaryl (e.g., 5 to 10 membered, 5 to 9 membered, or 5 to 6 membered). X³⁹ is —F, —Cl, —Br, or —I.

In embodiments, R^{7A} is independently hydrogen, —CX^{7A}₃, —CN, —COOH, —CONH₂, —CHX^{7A}₂, —CH₂X^{7A}, R^{38A}-substituted or unsubstituted alkyl, R^{38A}-substituted or unsubstituted heteroalkyl, R^{38A}-substituted or unsubstituted cycloalkyl, R^{38A}-substituted or unsubstituted heterocycloalkyl, R^{38A}-substituted or unsubstituted aryl, or R^{38A}-substituted or unsubstituted heteroaryl. In embodiments, R^{7A} is independently hydrogen, —CX^{7A}₃, —CN, —COOH, —CONH₂, —CHX^{7A}₂, —CH₂X^{7A}, R^{38A}-substituted or unsubstituted C₁-C₈ alkyl, R^{38A}-substituted or unsubstituted 2 to 8 membered heteroalkyl, R^{38A}-substituted or unsubstituted C₃-C₈ cycloalkyl, R^{38A}-substituted or unsubstituted 3 to 6 membered heterocycloalkyl, R^{38A}-substituted or unsubstituted phenyl, or R^{38A}-substituted or unsubstituted 5 to 6 membered heteroaryl. X^{7A} is —F, —Cl, —Br, or —I. In embodiments, R^{7A} is independently hydrogen. In embodiments, R^{7A} is independently methyl. In embodiments, R^{7A} is independently ethyl.

In embodiments, R^{7A} and R^{7B} substituents bonded to the same nitrogen atom may optionally be joined to form a R^{38A}-substituted or unsubstituted heterocycloalkyl or R^{38A}-substituted or unsubstituted heteroaryl. In embodiments, R^{7A} and R^{7B} substituents bonded to the same nitrogen atom may optionally be joined to form a R^{38A}-substituted or unsubstituted 3 to 6

R^{38A} is independently oxo, halogen, —CX^{38A}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{38A}₃, —OCHX^{38A}₂, R^{39A}-substituted or unsubstituted alkyl (e.g., C₁-C₈, C₁-C₆, or C₁-C₄), R^{39A}-substituted or unsubstituted heteroalkyl (e.g., 2 to 8 membered, 2 to 6 membered, or 2 to 4 membered), R^{39A}-substituted or unsubstituted cycloalkyl (e.g., C₃-C₈, C₃-C₆, or C₅-C₆), R^{39A}-substituted or unsubstituted heterocycloalkyl (e.g., 3 to 8 membered, 3 to 6 membered, or 5 to 6 membered), R^{39A}-substituted or unsubstituted aryl (e.g., C₆-C₁₀, C₁₀, or phenyl), or R^{39A}-substituted or unsubstituted heteroaryl (e.g., 5 to 10 membered, 5 to 9 membered, or 5 to 6 membered). X^{38A} is —F, —Cl, —Br, or —I.

R^{39A} is independently oxo, halogen, —CX^{39A}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{39A}₃, —OCHX^{39A}₂, R^{40A}-substituted or unsubstituted alkyl (e.g., C₁-C₈, C₁-C₆, or C₁-C₄), R^{40A}-substituted or unsubstituted heteroalkyl (e.g., 2 to 8 membered, 2 to 6 membered, or 2 to 4 membered), R^{40A}-substituted or unsubstituted cycloalkyl (e.g., C₃-C₈, C₃-C₆, or C₅-C₆), R^{40A}-substituted or unsubstituted heterocycloalkyl (e.g., 3 to 8 membered, 3 to 6 membered, or 5 to 6 membered), R^{40A}-substituted or unsubstituted aryl (e.g., C₆-C₁₀, C₁₀, or phenyl), or R^{40A}-substituted or unsubstituted heteroaryl (e.g., 5 to 10 membered, 5 to 9 membered, or 5 to 6 membered). X^{39A} is —F, —Cl, —Br, or —I.

In embodiments, R^{7B} is independently hydrogen, —CX^{7B}₃, —CN, —COOH, —CONH₂, —CHX^{7B}₂, —CH₂X^{7B}, R^{38B}-substituted or unsubstituted alkyl, R^{38B}-substituted or unsubstituted heteroalkyl, R^{38B}-substituted or unsubstituted cycloalkyl, R^{38B}-substituted or unsubstituted heterocycloalkyl, R^{38B}-substituted or unsubstituted aryl, or R^{38B}-substituted or unsubstituted heteroaryl. In embodiments, R^{7B} is independently hydrogen, —CX^{7B}₃, —CN, —COOH, —CONH₂, —CHX^{7B}₂, —CH₂X^{7B}, R^{38B}-substituted or unsubstituted C₁-C₈ alkyl, R^{38B}-substituted or unsubstituted 2 to 8 membered heteroalkyl, R^{38B}-substituted or unsubstituted C₃-C₈ cycloalkyl, R^{38B}-substituted or unsubstituted 3 to 6 membered heterocycloalkyl, R^{38B}-substituted or unsubstituted phenyl, or R^{38B}-substituted or unsubstituted 5 to 6 membered heteroaryl. X^{7B} is —F, —Cl, —Br, or —I. In embodiments, R^{7B} is independently hydrogen. In embodiments, R^{7B} is independently methyl. In embodiments, R^{7B} is independently ethyl.

In embodiments, R^{7A} and R^{7B} substituents bonded to the same nitrogen atom may optionally be joined to form a R^{38B}-substituted or unsubstituted heterocycloalkyl or R^{38B}-substituted or unsubstituted heteroaryl. In embodiments, R^{7A} and R^{7B} substituents bonded to the same nitrogen atom may optionally be joined to form a R^{38B}-substituted or unsubstituted 3 to 6 membered heterocycloalkyl or R^{38B}-substituted or unsubstituted 5 to 6 membered heteroaryl.

R^{38B} is independently oxo, halogen, —CX^{38B}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{38B}₃, —OCHX^{38B}₂, R^{39B}-substituted or unsubstituted alkyl (e.g., C₁-C₈, C₁-C₆, or C₁-C₄), R^{39B}-substituted or unsubstituted heteroalkyl (e.g., 2 to 8 membered, 2 to 6 membered, or 2 to 4 membered), R^{39B}-substituted or unsubstituted cycloalkyl (e.g., C₃-C C₃-C₆, or C₅-C₆), R^{39B}-substituted or unsubstituted heterocycloalkyl (e.g., 3 to 8 membered, 3 to 6 membered, or 5 to 6 membered), R^{39B}-substituted or unsubstituted aryl (e.g., C₆-C₁₀, C₁₀, or phenyl), or R^{39B}-substituted or unsubstituted heteroaryl (e.g., 5 to 10 membered, 5 to 9 membered, or 5 to 6 membered). X^{38B} is —F, —Cl, —Br, or —I.

R^{39B} is independently oxo, halogen, —CX^{39B}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{39B}₃, —OCHX^{39B}₂, R^{40B}-substituted or unsubstituted alkyl (e.g., C₁-C₈, C₁-C₆, or C₁-C₄), R^{40B}-substituted or unsubstituted heteroalkyl (e.g., 2 to 8 membered, 2 to 6 membered, or 2 to 4 membered), R^{40B}-substituted or unsubstituted cycloalkyl (e.g., C₃-C C₃-C₆, or C₅-C₆), R^{40B}-substituted or unsubstituted heterocycloalkyl (e.g., 3 to 8 membered, 3 to 6 membered, or 5 to 6 membered), R^{40B}-substituted or unsubstituted aryl (e.g., C₆-C₁₀, C₁₀, or phenyl), or R^{40B}-substituted or unsubstituted heteroaryl (e.g., 5 to 10 membered, 5 to 9 membered, or 5 to 6 membered). X^{39B} is —F, —Cl, —Br, or —I.

In embodiments, R^{7C} is independently hydrogen, —CX^{7C}₃, —CN, —COOH, —CONH₂, —CHX^{7C}₂, —CH₂X^{7C}, R^{38C}-substituted or unsubstituted alkyl, R^{38C}-substituted or unsubstituted heteroalkyl, R^{38C}-substituted or unsubstituted cycloalkyl, R^{38C}-substituted or unsubstituted heterocycloalkyl, R^{38C}-substituted or unsubstituted aryl, or R^{38C}-substituted or unsubstituted heteroaryl. In embodiments, R^{7C} is independently hydrogen, —CX^{7C}₃, —CN, —COOH, —CONH₂, —CHX^{7C}₂, —CH₂X^{7C}, R^{38C}-substituted or unsubstituted C₁-C₈ alkyl, R^{38C}-substituted or unsubstituted 2 to 8 membered heteroalkyl, R^{38C}-substituted or unsubstituted C₃-C₈ cycloalkyl, R^{38C}-substituted or unsubstituted 3 to 6 membered heterocycloalkyl, R^{38C}-substituted or unsubstituted phenyl, or R^{38C}-substituted or unsubstituted 5 to 6 membered heteroaryl. X^{7C} is —F, —Cl, —Br, or —I. In embodiments, R^{7C} is independently hydrogen. In embodiments, R^{7C} is independently methyl. In embodiments, R^{7C} is independently ethyl.

R^{38C} is independently oxo, halogen, —CX^{38C}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{38C}₃, —OCHX^{38C}₂, R^{39C}-substituted or unsubstituted alkyl (e.g., C₁-C₈, C₁-C₆, or C₁-C₄), R^{39C}-substituted or unsubstituted heteroalkyl (e.g., 2 to 8 membered, 2 to 6 membered, or 2 to 4 membered), R^{39C}-substituted or unsubstituted cycloalkyl (e.g., C₃-C₈, C₃-C₆, or C₅-C₆), R^{39C}-substituted or unsubstituted heterocycloalkyl (e.g., 3 to 8 membered, 3 to 6 membered, or 5 to 6 membered), R^{39C}-substituted or unsubstituted aryl (e.g., C₆-C₁₀, C₁₀, or phenyl), or R^{39C}-substituted or unsubstituted heteroaryl (e.g., 5 to 10 membered, 5 to 9 membered, or 5 to 6 membered). X^{38C} is —F, —Cl, —Br, or —I.

R^{39C} is independently oxo, halogen, —CX^{39C}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{39C}₃, —OCHX^{39C}₂, R^{40C}-substituted or unsubstituted alkyl (e.g., C₁-C₈, C₁-C₆, or C₁-C₄), R^{40C}-substituted or unsubstituted heteroalkyl (e.g., 2 to 8 membered, 2 to 6 membered, or 2 to 4 membered), R^{40C}-substituted or unsubstituted cycloalkyl (e.g., C₃-C₈, C₃-C₆, or C₅-C₆), R^{40C}-substituted or unsubstituted heterocycloalkyl (e.g., 3 to 8 membered, 3 to 6 membered, or 5 to 6 membered), R^{40C}-substituted or unsubstituted aryl (e.g., C₆-C₁₀, C₁₀, or phenyl), or R^{40C}-substituted or unsubstituted heteroaryl (e.g., 5 to 10 membered, 5 to 9 membered, or 5 to 6 membered). X^{39C} is —F, —Cl, —Br, or —I.

In embodiments, R^{7D} is independently hydrogen, —CX^{7D}₃, —CN, —COOH, —CONH₂, —CHX^{7D}₂, —CH₂X^{7D}, R^{38D}-substituted or unsubstituted alkyl, R^{38D}-substituted or unsubstituted heteroalkyl, R^{38D}-substituted or unsubstituted cycloalkyl, R^{38D}-substituted or unsubstituted heterocycloalkyl, R^{38D}-substituted or unsubstituted aryl, or R^{38D}-substituted or unsubstituted heteroaryl. In embodiments, R^{7D} is independently hydrogen, —CX^{7D}₃, —CN, —COOH, —CONH₂, —CHX^{7D}₂, —CH₂X^{7D}, R^{38D}-substituted or unsubstituted C₁-C₈ alkyl, R^{38D}-substituted or unsubstituted 2 to 8 membered heteroalkyl, R^{38D}-substituted or unsubstituted C₃-C₈ cycloalkyl, R^{38D}-substituted or unsubstituted 3 to 6 membered heterocycloalkyl, R^{38D}-substituted or unsubstituted phenyl, or R^{38D}-substituted or unsubstituted 5 to 6 membered heteroaryl. X^{7D} is —F, —Cl, —Br, or —I. In embodiments, R^{7D} is independently hydrogen. In embodiments, R^{7D} is independently methyl. In embodiments, R^{7D} is independently ethyl.

R^{38D} is independently oxo, halogen, —CX^{38D}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{38D}₃, —OCHX^{38D}₂, R^{39D}-substituted or unsubstituted alkyl (e.g., C₁-C₈, C₁-C₆, or C₁-C₄), R^{39D}-substituted or unsubstituted heteroalkyl (e.g., 2 to 8 membered, 2 to 6 membered, or 2 to 4 membered), R^{39D}-substituted or unsubstituted cycloalkyl (e.g., C₃-C₈, C₃-C₆, or C₅-C₆), R^{39D}-substituted or unsubstituted heterocycloalkyl (e.g., 3 to 8 membered, 3 to 6 membered, or 5 to 6 membered), R^{39D}-substituted or unsubstituted aryl (e.g., C₆-C₁₀, C₁₀, or phenyl), or R^{39D}-substituted or unsubstituted heteroaryl (e.g., 5 to 10 membered, 5 to 9 membered, or 5 to 6 membered). X^{38D} is —F, —Cl, —Br, or —I.

R^{39D} is independently oxo, halogen, —CX^{39D}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{39D}₃, —OCHX^{39D}₂, R^{40D}-substituted or unsubstituted alkyl (e.g., C₁-C₈, C₁-C₆, or C₁-C₄), R^{40D}-substituted or unsubstituted heteroalkyl (e.g., 2 to 8 membered, 2 to 6 membered, or 2 to 4 membered), R^{40D}-substituted or unsubstituted cycloalkyl (e.g., C₃-C₈, C₃-C₆, or C₅-C₆), R^{40D}-substituted or unsubstituted heterocycloalkyl (e.g., 3 to 8 membered, 3 to 6 membered, or 5 to 6 membered), R^{40D}-substituted or unsubstituted aryl (e.g., C₆-C₁₀, C₁₀, or phenyl), or R^{40D}-substituted or unsubstituted heteroaryl (e.g., 5 to 10 membered, 5 to 9 membered, or 5 to 6 membered). X^{39D} is —F, —Cl, —Br, or —I.

R⁴⁰, R^{40A}, R^{40B}, R^{40C}, and R^{40D} are independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, L³ is a bond. In embodiments, L³ is —S(O)₂—. In embodiments, L³ is —NR⁸—. In embodiments, L³ is —O—. In embodiments, L³ is —S—. In embodiments, L³ is —C(O)—. In embodiments, L³ is —C(O)NR⁸—. In embodiments, L³ is —NR⁸C(O)—. In embodiments, L³ is —NR⁸C(O)NH—. In embodiments, L³ is —NHC(O)NR⁸—. In embodiments, L³ is —C(O)O—. In embodiments, L³ is —OC(O)—. In embodiments, L³ is —NH—. In embodiments, L³ is —C(O)NH—. In embodiments, L³ is —NHC(O)—. In embodiments, L³ is —NHC(O)NH—.

In embodiments, L³ is a substituted or unsubstituted alkylene. In embodiments, L³ is a substituted or unsubstituted heteroalkylene. In embodiments, L³ is a substituted alkylene. In embodiments, L³ is a substituted heteroalkylene. In embodiments, L³ is an unsubstituted alkylene. In embodiments, L³ is an unsubstituted heteroalkylene. In embodiments, L³ is a substituted or unsubstituted C₁-C₈ alkylene. In embodiments, L³ is a substituted or unsubstituted 2 to 8 membered heteroalkylene. In embodiments, L³ is a substituted C₁-C₈ alkylene. In embodiments, L³ is a substituted 2 to 8 membered heteroalkylene. In embodiments, L³ is an unsubstituted C₁-C₈ alkylene. In embodiments, L³ is an unsubstituted 2 to 8 membered heteroalkylene. In embodiments, L³ is a substituted or unsubstituted C₁-C₄ alkylene. In embodiments, L³ is a substituted or unsubstituted 2 to 4 membered heteroalkylene. In embodiments, L³ is a substituted C₁-C₄ alkylene. In embodiments, L³ is a substituted 2 to 4 membered heteroalkylene. In embodiments, L³ is an unsubstituted C₁-C₄ alkylene. In embodiments, L³ is an unsubstituted 2 to 4 membered heteroalkylene.

In embodiments, L³ is a bond, —S(O)₂—, —S(O)₂-Ph-, —O—, —S—, —C(O)—, —C(O)NR⁸—, —NR⁸C(O)—, —NR⁸C(O)NH—, —NHC(O)NR⁸—, —C(O)O—, —OC(O)—, R⁵⁰-substituted or unsubstituted alkylene, or R⁵⁰-substituted or unsubstituted heteroalkylene. In embodiments, L³ is a bond, —S(O)₂—, —S(O)₂-Ph-, —NH—, —O—, —S—, —C(O)—, —C(O)NH—, —NHC(O)—, —NHC(O)NH—, —C(O)O—, —OC(O)—, R⁵⁰-substituted or unsubstituted alkylene, or R⁵⁰-substituted or unsubstituted heteroalkylene. In embodiments, L³ is a bond, —S(O)₂—, —S(O)₂-Ph-, —NH—, —O—, —S—, —C(O)—, —C(O)NH—, —NHC(O)—, —NHC(O)NH—, —C(O)O—, —OC(O)—, R⁵⁰-substituted or unsubstituted C₁-C₈ alkylene, or R⁵⁰-substituted or unsubstituted 2 to 8 membered heteroalkylene.

R⁵⁰ is independently oxo, halogen, —CX⁵⁰₃, —CHX⁵⁰₂, —CH₂X⁵⁰, —OCX⁵⁰₃, —OCHX⁵⁰₂, —OCH₂X⁵⁰, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁵¹-substituted or unsubstituted alkyl (e.g., C₁-C₈, C₁-C₆, or C₁-C₄), R⁵¹-substituted or unsubstituted heteroalkyl (e.g., 2 to 8 membered, 2 to 6 membered, or 2 to 4 membered), R⁵¹-substituted or unsubstituted cycloalkyl (e.g., C₃-C₈, C₃-C₆, or C₅-C₆), R⁵¹-substituted or unsubstituted heterocycloalkyl (e.g., 3 to 8 membered, 3 to 6 membered, or 5 to 6 membered), R⁵¹-substituted or unsubstituted aryl (e.g., C₆-C₁₀, C₁₀, or phenyl), or R⁵¹-substituted or unsubstituted heteroaryl (e.g., 5 to 10 membered, 5 to 9 membered, or 5 to 6 membered). X⁵⁰ is —F, —Cl, —Br, or —I.

R⁵¹ is independently oxo, halogen, —CX⁵¹₃, —CHX⁵¹₂, —CH₂X⁵¹, —OCX⁵¹₃, —OCHX⁵¹₂, —OCH₂X⁵¹, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁵²-substituted or unsubstituted alkyl (e.g., C₁-C₈, C₁-C₆, or C₁-C₄), R⁵²-substituted or unsubstituted heteroalkyl (e.g., 2 to 8 membered, 2 to 6 membered, or 2 to 4 membered), R⁵²-substituted or unsubstituted cycloalkyl (e.g., C₃-C₈, C₃-C₆, or C₅-C₆), R⁵²-substituted or unsubstituted heterocycloalkyl (e.g., 3 to 8 membered, 3 to 6 membered, or 5 to 6 membered), R⁵²-substituted or unsubstituted aryl (e.g., C₆-C₁₀, C₁₀, or phenyl), or R⁵²-substituted or unsubstituted heteroaryl (e.g., 5 to 10 membered, 5 to 9 membered, or 5 to 6 membered). X⁵¹ is —F, —Cl, —Br, or —I.

R⁵² is independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCl₃, —OCHF₂, —OCHCl₂, OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, L³ is R⁵⁰-substituted or unsubstituted C₁-C₂ alkylene. In embodiments, L³ is R⁵⁰-substituted or unsubstituted C₁-C₄ alkylene. In embodiments, L³ is R⁵⁰-substituted or unsubstituted C₁-C₆ alkylene. In embodiments, L³ is R⁵⁰-substituted or unsubstituted C₁-C₈ alkylene. In embodiments, L³ is R⁵⁰-substituted or unsubstituted alkylene (e.g., C₁-C₈ alkylene, C₁-C₆ alkylene, C₁-C₄ alkylene, C₁-C₂ alkylene). In embodiments, L³ is R⁵⁰-substituted C₁-C₂ alkylene. In embodiments, L³ is R⁵⁰-substituted C₁-C₄ alkylene. In embodiments, L³ is R⁵⁰-substituted C₁-C₆ alkylene. In embodiments, L³ is R⁵⁰-substituted C₁-C₈ alkylene. In embodiments, L³ is R⁵⁰-substituted alkylene (e.g., C₁-C₈ alkylene, C₁-C₆ alkylene, C₁-C₄ alkylene, C₁-C₂ alkylene). In embodiments, L³ is R⁵⁰-substituted methylene. In embodiments, L³ is an unsubstituted C₁-C₂ alkylene. In embodiments, L³ is an unsubstituted C₁-C₄ alkylene. In embodiments, L³ is an unsubstituted C₁-C₆ alkylene. In embodiments, L³ is an unsubstituted C₁-C₈ alkylene. In embodiments, L³ is an unsubstituted alkylene (e.g., C₁-C₈ alkylene, C₁-C₆ alkylene, C₁-C₄ alkylene, C₁-C₂ alkylene). In embodiments, L³ is R⁵⁰-substituted or unsubstituted methylene. In embodiments, L³ is R⁵⁰-substituted methylene. In embodiments, L³ is an unsubstituted methylene. In embodiments, L³ is a bond.

In embodiments, L³ is R⁵⁰-substituted or unsubstituted 2 to 4 membered heteroalkylene. In embodiments, L³ is R⁵⁰-substituted or unsubstituted 2 to 6 membered heteroalkylene. In embodiments, L³ is R⁵⁰-substituted or unsubstituted 2 to 8 membered heteroalkylene. In embodiments, L³ is R⁵⁰-substituted or unsubstituted heteroalkylene (e.g., 2 to 8 membered heteroalkylene, 2 to 6 membered heteroalkylene, 2 to 4 membered heteroalkylene). In embodiments, L³ is R⁵⁰-substituted 2 to 4 membered heteroalkylene. In embodiments, L³ is R⁵⁰-substituted 2 to 6 membered heteroalkylene. In embodiments, L³ is R⁵⁰-substituted 2 to 8 membered heteroalkylene. In embodiments, L³ is R⁵⁰-substituted heteroalkylene (e.g., 2 to 8 membered heteroalkylene, 2 to 6 membered heteroalkylene, 2 to 4 membered heteroalkylene). In embodiments, L³ is an unsubstituted 2 to 4 membered heteroalkylene. In embodiments, L³ is an unsubstituted 2 to 6 membered heteroalkylene. In embodiments, L³ is an unsubstituted 2 to 8 membered heteroalkylene. In embodiments, L³ is an unsubstituted heteroalkylene (e.g., 2 to 8 membered heteroalkylene, 2 to 6 membered heteroalkylene, 2 to 4 membered heteroalkylene).

In embodiments, R⁸ is substituted or unsubstituted C₁-C₆ alkyl. In embodiments, R⁸ is substituted or unsubstituted 2 to 6 membered heteroalkyl. In embodiments, R⁸ is substituted or unsubstituted C₃-C₆ cycloalkyl. In embodiments, R⁸ is substituted or unsubstituted 3 to 6 membered heterocycloalkyl. In embodiments, R⁸ is substituted or unsubstituted phenyl. In embodiments, R⁸ is substituted or unsubstituted 5 to 6 membered heteroaryl. In embodiments, R⁸ is substituted C₁-C₆ alkyl. In embodiments, R⁸ is substituted 2 to 6 membered heteroalkyl. In embodiments, R⁸ is substituted C₃-C₆ cycloalkyl. In embodiments, R⁸ is substituted 3 to 6 membered heterocycloalkyl. In embodiments, R⁸ is substituted phenyl. In embodiments, R⁸ is substituted 5 to 6 membered heteroaryl. In embodiments, R⁸ is an unsubstituted C₁-C₆ alkyl. In embodiments, R⁸ is an unsubstituted 2 to 6 membered heteroalkyl. In embodiments, R⁸ is an unsubstituted C₃-C₆ cycloalkyl. In embodiments, R⁸ is an unsubstituted 3 to 6 membered heterocycloalkyl. In embodiments, R⁸ is an unsubstituted phenyl. In embodiments, R⁸ is an unsubstituted 5 to 6 membered heteroaryl.

In embodiments, R⁸ is substituted or unsubstituted C₁-C₄ alkyl. In embodiments, R⁸ is substituted or unsubstituted C₃-C₅ cycloalkyl. In embodiments, R⁸ is substituted or unsubstituted 3 to 5 membered heterocycloalkyl. In embodiments, R⁸ is substituted or unsubstituted 5 membered heteroaryl. In embodiments, R⁸ is substituted C₁-C₄ alkyl. In embodiments, R⁸ is substituted C₃-C₅ cycloalkyl. In embodiments, R⁸ is substituted 3 to 5 membered heterocycloalkyl. In embodiments, R⁸ is substituted 5 membered heteroaryl. In embodiments, R⁸ is substituted 6 membered heteroaryl. In embodiments, R⁸ is an unsubstituted C₁-C₄ alkyl. In embodiments, R⁸ is an unsubstituted C₃-C₅ cycloalkyl. In embodiments, R⁸ is an unsubstituted 3 to 5 membered heterocycloalkyl. In embodiments, R⁸ is an unsubstituted 5 membered heteroaryl. In embodiments, R⁸ is an unsubstituted 6 membered heteroaryl. In embodiments, R⁸ is substituted C₁-C₃ alkyl. In embodiments, R⁸ is an unsubstituted C₁-C₃ alkyl.

In embodiments, R⁸ is independently hydrogen. In embodiments, R⁸ is independently halogen. In embodiments, R⁸ is independently —CX⁸₃. In embodiments, R⁸ is independently —CHX⁸₂. In embodiments, R⁸ is independently —CH₂X⁸. In embodiments, R⁸ is independently —OCX⁸₃. In embodiments, R⁸ is independently —OCH₂X⁸. In embodiments, R⁸ is independently —OCHX⁸₂. In embodiments, R⁸ is independently —CN. In embodiments, R⁸ is independently —SOₙ₈R^{8D}. In embodiments, R⁸ is independently —SOᵥ₈NR^{8A}R^{8B}. In embodiments, R⁸ is independently —NHC(O)NR^{8A}R^{8B}. In embodiments, R⁸ is independently —N(O)ₘ₈. In embodiments, R⁸ is independently —NR^{8A}R^{8B}. In embodiments, R⁸ is independently —C(O)R^{8C}. In embodiments, R⁸ is independently —C(O)—OR^{8C}. In embodiments, R⁸ is independently —C(O)NR^{8A}R^{8B}. In embodiments, R⁸ is independently —OR^{8D}. In embodiments, R⁸ is independently —NR^{8A}SO₂R^{8D}. In embodiments, R⁸ is independently —NR^{8A}C(O)R^{8C}. In embodiments, R⁸ is independently —NR^{8A}C(O)OR^{8C}. In embodiments, R⁸ is independently —NR^{8A}OR^{8C}. In embodiments, R⁸ is independently —OH. In embodiments, R⁸ is independently —NH₂. In embodiments, R⁸ is independently —COOH. In embodiments, R⁸ is independently —CONH₂. In embodiments, R⁸ is independently —NO₂. In embodiments, R⁸ is independently —SH.

In embodiments, R⁸ is independently substituted or unsubstituted alkyl (e.g., C₁-C₈, C₁-C₆, or C₁-C₄). In embodiments, R⁸ is independently substituted alkyl (e.g., C₁-C₈, C₁-C₆, or C₁-C₄). In embodiments, R⁸ is independently unsubstituted alkyl (e.g., C₁-C₈, C₁-C₆, or C₁-C₄). In embodiments, R⁸ is independently substituted or unsubstituted heteroalkyl (e.g., 2 to 8 membered, 2 to 6 membered, or 2 to 4 membered). In embodiments, R⁸ is independently substituted heteroalkyl (e.g., 2 to 8 membered, 2 to 6 membered, or 2 to 4 membered). In embodiments, R⁸ is independently unsubstituted heteroalkyl (e.g., 2 to 8 membered, 2 to 6 membered, or 2 to 4 membered). In embodiments, R⁸ is independently substituted or unsubstituted cycloalkyl (e.g., C₃-C₈, C₃-C₆, or C₅-C₆). In embodiments, R⁸ is independently substituted cycloalkyl (e.g., C₃-C₈, C₃-C₆, or C₅-C₆). In embodiments, R⁸ is independently unsubstituted cycloalkyl (e.g., C₃-C₈, C₃-C₆, or C₅-C₆). In embodiments, R⁸ is independently substituted or unsubstituted heterocycloalkyl (e.g., 3 to 8 membered, 3 to 6 membered, or 5 to 6 membered). In embodiments, R⁸ is independently substituted heterocycloalkyl (e.g., 3 to 8 membered, 3 to 6 membered, or 5 to 6 membered). In embodiments, R⁸ is independently unsubstituted heterocycloalkyl (e.g., 3 to 8 membered, 3 to 6 membered, or 5 to 6 membered). In embodiments, R⁸ is independently substituted or unsubstituted aryl (e.g., C₆-C₁₀, C₁₀, or phenyl). In embodiments, R⁸ is independently substituted aryl (e.g., C₆-C₁₀, C₁₀), or phenyl). In embodiments, R⁸ is independently unsubstituted aryl (e.g., C₆-C₁₀, C₁₀, or phenyl). In embodiments, R⁸ is independently substituted or unsubstituted heteroaryl (e.g., 5 to 10 membered, 5 to 9 membered, or 5 to 6 membered). In embodiments, R⁸ is independently substituted heteroaryl (e.g., 5 to 10 membered, 5 to 9 membered, or 5 to 6 membered). In embodiments, R⁸ is independently unsubstituted heteroaryl (e.g., 5 to 10 membered, 5 to 9 membered, or 5 to 6 membered).

In embodiments, R^{8A} is independently hydrogen. In embodiments, R^{8A} is independently —CX^{8A}₃. In embodiments, R^{8A} is independently —CHX^{8A}₂. In embodiments, R^{8A} is independently —CH₂X^{8A}. In embodiments, R^{8A} is independently —CN. In embodiments, R^{8A} is independently —COOH. In embodiments, R^{8A} is independently —CONH₂.

In embodiments, R^{8A} is independently substituted or unsubstituted alkyl (e.g., C₁-C₈, C₁-C₆, or C₁-C₄). In embodiments, R^{8A} is independently substituted alkyl (e.g., C₁-C₈, C₁-C₆, or C₁-C₄). In embodiments, R^{8A} is independently unsubstituted alkyl (e.g., C₁-C₈, C₁-C₆, or C₁-C₄). In embodiments, R^{8A} is independently substituted or unsubstituted heteroalkyl (e.g., 2 to 8 membered, 2 to 6 membered, or 2 to 4 membered). In embodiments, R^{8A} is independently substituted heteroalkyl (e.g., 2 to 8 membered, 2 to 6 membered, or 2 to 4 membered). In embodiments, R^{8A} is independently unsubstituted heteroalkyl (e.g., 2 to 8 membered, 2 to 6 membered, or 2 to 4 membered). In embodiments, R^{8A} is independently substituted or unsubstituted cycloalkyl (e.g., C₃-C₈, C₃-C₆, or C₅-C₆). In embodiments, R^{8A} is independently substituted cycloalkyl (e.g., C₃-C₈, C₃-C₆, or C₅-C₆). In embodiments, R^{8A} is independently unsubstituted cycloalkyl (e.g., C₃-C₈, C₃-C₆, or C₅-C₆). In embodiments, R^{8A} is independently substituted or unsubstituted heterocycloalkyl (e.g., 3 to 8 membered, 3 to 6 membered, or 5 to 6 membered). In embodiments, R^{8A} is independently substituted heterocycloalkyl (e.g., 3 to 8 membered, 3 to 6 membered, or 5 to 6 membered). In embodiments, R^{8A} is independently unsubstituted heterocycloalkyl (e.g., 3 to 8 membered, 3 to 6 membered, or 5 to 6 membered). In embodiments, R^{8A} is independently substituted or unsubstituted aryl (e.g., C₆-C₁₀, C₁₀, or phenyl). In embodiments, R^{8A} is independently substituted aryl (e.g., C₆-C₁₀, C₁₀, or phenyl). In embodiments, R^{8A} is independently unsubstituted aryl (e.g., C₆-C₁₀, C₁₀, or phenyl). In embodiments, R^{8A} is independently substituted or unsubstituted heteroaryl (e.g., 5 to 10 membered, 5 to 9 membered, or 5 to 6 membered). In embodiments, R^{8A} is independently substituted heteroaryl (e.g., 5 to 10 membered, 5 to 9 membered, or 5 to 6 membered). In embodiments, R^{8A} is independently unsubstituted heteroaryl (e.g., 5 to 10 membered, 5 to 9 membered, or 5 to 6 membered). In embodiments, R^{8A} is independently unsubstituted methyl. In embodiments, R^{8A} is independently unsubstituted ethyl. In embodiments, R^{8A} is independently unsubstituted propyl. In embodiments, R^{8A} is independently unsubstituted isopropyl. In embodiments, R^{8A} is independently unsubstituted tert-butyl.

In embodiments, R^{8B} is independently hydrogen. In embodiments, R^{8B} is independently —CX^{8B}₃. In embodiments, R^{8B} is independently —CHX^{8B}₂. In embodiments, R^{8B} is independently —CH₂X^{8B}. In embodiments, R^{8B} is independently —CN. In embodiments, R^{8B} is independently —COOH. In embodiments, R^{8B} is independently —CONH₂.

In embodiments, R^{8B} is independently substituted or unsubstituted alkyl (e.g., C₁-C₈, C₁-C₆, or C₁-C₄). In embodiments, R^{8B} is independently substituted alkyl (e.g., C₁-C₈, C₁-C₆, or C₁-C₄). In embodiments, R^{8B} is independently unsubstituted alkyl (e.g., C₁-C₈, C₁-C₆, or C₁-C₄). In embodiments, R^{8B} is independently substituted or unsubstituted heteroalkyl (e.g., 2 to 8 membered, 2 to 6 membered, or 2 to 4 membered). In embodiments, R^{8B} is independently substituted heteroalkyl (e.g., 2 to 8 membered, 2 to 6 membered, or 2 to 4 membered). In embodiments, R^{8B} is independently unsubstituted heteroalkyl (e.g., 2 to 8 membered, 2 to 6 membered, or 2 to 4 membered). In embodiments, R^{8B} is independently substituted or unsubstituted cycloalkyl (e.g., C₃-C₈, C₃-C₆, or C₅-C₆). In embodiments, R^{8B} is independently substituted cycloalkyl (e.g., C₃-C₈, C₃-C₆, or C₅-C₆). In embodiments, R^{8B} is independently unsubstituted cycloalkyl (e.g., C₃-C₈, C₃-C₆, or C₅-C₆). In embodiments, R^{8B} is independently substituted or unsubstituted heterocycloalkyl (e.g., 3 to 8 membered, 3 to 6 membered, or 5 to 6 membered). In embodiments, R^{8B} is independently substituted heterocycloalkyl (e.g., 3 to 8 membered, 3 to 6 membered, or 5 to 6 membered). In embodiments, R^{8B} is independently unsubstituted heterocycloalkyl (e.g., 3 to 8 membered, 3 to 6 membered, or 5 to 6 membered). In embodiments, R^{8B} is independently substituted or unsubstituted aryl (e.g., C₆-C₁₀, C₁₀, or phenyl). In embodiments, R^{8B} is independently substituted aryl (e.g., C₆-C₁₀, C₁₀, or phenyl). In embodiments, R^{8B} is independently unsubstituted aryl (e.g., C₆-C₁₀, C₁₀, or phenyl). In embodiments, R^{8B} is independently substituted or unsubstituted heteroaryl (e.g., 5 to 10 membered, 5 to 9 membered, or 5 to 6 membered). In embodiments, R^{8B} is independently substituted heteroaryl (e.g., 5 to 10 membered, 5 to 9 membered, or 5 to 6 membered). In embodiments, R^{8B} is independently unsubstituted heteroaryl (e.g., 5 to 10 membered, 5 to 9 membered, or 5 to 6 membered). In embodiments, R^{8B} is independently unsubstituted methyl. In embodiments, R^{8B} is independently unsubstituted ethyl. In embodiments, R^{8B} is independently unsubstituted propyl. In embodiments, R^{8B} is independently unsubstituted isopropyl. In embodiments, R^{8B} is independently unsubstituted tert-butyl.

In embodiments, R^{8A} and R^{8B} substituents bonded to the same nitrogen atom may be joined to form a substituted or unsubstituted heterocycloalkyl. In embodiments, R^{8A} and R^{8B} substituents bonded to the same nitrogen atom may be joined to form a substituted or unsubstituted heteroaryl. In embodiments, R^{8A} and R^{8B} substituents bonded to the same nitrogen atom may be joined to form a substituted heterocycloalkyl. In embodiments, R^{8A} and R^{8B} substituents bonded to the same nitrogen atom may be joined to form a substituted heteroaryl. In embodiments, R^{8A} and R^{8B} substituents bonded to the same nitrogen atom may be joined to form an unsubstituted heterocycloalkyl. In embodiments, R^{8A} and R^{8B} substituents bonded to the same nitrogen atom may be joined to form an unsubstituted heteroaryl. In embodiments, R^{8A} and R^{8B} substituents bonded to the same nitrogen atom may be joined to form a substituted or unsubstituted 3 to 8 membered heterocycloalkyl. In embodiments, R^{8A} and R^{8B} substituents bonded to the same nitrogen atom may be joined to form a substituted or unsubstituted 5 to 10 membered heteroaryl. In embodiments, R^{8A} and R^{8B} substituents bonded to the same nitrogen atom may be joined to form a substituted 3 to 8 membered heterocycloalkyl. In embodiments, R^{8A} and R^{8B} substituents bonded to the same nitrogen atom may be joined to form a substituted 5 to 10 membered heteroaryl. In embodiments, R^{8A} and R^{8B} substituents bonded to the same nitrogen atom may be joined to form an unsubstituted 3 to 8 membered heterocycloalkyl. In embodiments, R^{8A} and R^{8B} substituents bonded to the same nitrogen atom may be joined to form an unsubstituted 5 to 10 membered heteroaryl. In embodiments, R^{8A} and R^{8B} substituents bonded to the same nitrogen atom may be joined to form a substituted or unsubstituted 3 to 6 membered heterocycloalkyl. In embodiments, R^{8A} and R^{8B} substituents bonded to the same nitrogen atom may be joined to form a substituted or unsubstituted 5 to 6 membered heteroaryl. In embodiments, R^{8A} and R^{8B} substituents bonded to the same nitrogen atom may be joined to form a substituted 3 to 6 membered heterocycloalkyl. In embodiments, R^{8A} and R^{8B} substituents bonded to the same nitrogen atom may be joined to form a substituted 5 to 6 membered heteroaryl. In embodiments, R^{8A} and R^{8B} substituents bonded to the same nitrogen atom may be joined to form an unsubstituted 3 to 6 membered heterocycloalkyl. In embodiments, R^{8A} and R^{8B} substituents bonded to the same nitrogen atom may be joined to form an unsubstituted 5 to 6 membered heteroaryl.

In embodiments, R^{8C} is independently hydrogen. In embodiments, R^{8C} is independently —CX^{8C}₃. In embodiments, R^{8C} is independently —CHX^{8C}₂. In embodiments, R^{8C} is independently —CH₂X^{8C}. In embodiments, R^{8C} is independently —CN. In embodiments, R^{8C} is independently —COOH. In embodiments, R^{8C} is independently —CONH₂.

In embodiments, R^{8C} is independently substituted or unsubstituted alkyl (e.g., C₁-C₈, C₁-C₆, or C₁-C₄). In embodiments, R^{8C} is independently substituted alkyl (e.g., C₁-C₈, C₁-C₆, or C₁-C₄). In embodiments, R^{8C} is independently unsubstituted alkyl (e.g., C₁-C₈, C₁-C₆, or C₁-C₄). In embodiments, R^{8C} is independently substituted or unsubstituted heteroalkyl (e.g., 2 to 8 membered, 2 to 6 membered, or 2 to 4 membered). In embodiments, R^{8C} is independently substituted heteroalkyl (e.g., 2 to 8 membered, 2 to 6 membered, or 2 to 4 membered). In embodiments, R^{8C} is independently unsubstituted heteroalkyl (e.g., 2 to 8 membered, 2 to 6 membered, or 2 to 4 membered). In embodiments, R^{8C} is independently substituted or unsubstituted cycloalkyl (e.g., C₃-C₈, C₃-C₆, or C₅-C₆). In embodiments, R^{8C} is independently substituted cycloalkyl (e.g., C₃-C₈, C₃-C₆, or C₅-C₆). In embodiments, R^{8C} is independently unsubstituted cycloalkyl (e.g., C₃-C₈, C₃-C₆, or C₅-C₆). In embodiments, R^{8C} is independently substituted or unsubstituted heterocycloalkyl (e.g., 3 to 8 membered, 3 to 6 membered, or 5 to 6 membered). In embodiments, R^{8C} is independently substituted heterocycloalkyl (e.g., 3 to 8 membered, 3 to 6 membered, or 5 to 6 membered). In embodiments, R^{8C} is independently unsubstituted heterocycloalkyl (e.g., 3 to 8 membered, 3 to 6 membered, or 5 to 6 membered). In embodiments, R^{8C} is independently substituted or unsubstituted aryl (e.g., C₆-C₁₀, C₁₀, or phenyl). In embodiments, R^{8C} is independently substituted aryl (e.g., C₆-C₁₀, C₁₀, or phenyl). In embodiments, R^{8C} is independently unsubstituted aryl (e.g., C₆-C₁₀, C₁₀, or phenyl). In embodiments, R^{8C} is independently substituted or unsubstituted heteroaryl (e.g., 5 to 10 membered, 5 to 9 membered, or 5 to 6 membered). In embodiments, R^{8C} is independently substituted heteroaryl (e.g., 5 to 10 membered, 5 to 9 membered, or 5 to 6 membered). In embodiments, R^{8C} is independently unsubstituted heteroaryl (e.g., 5 to 10 membered, 5 to 9 membered, or 5 to 6 membered). In embodiments, R^{8C} is independently unsubstituted methyl. In embodiments, R^{8C} is independently unsubstituted ethyl. In embodiments, R^{8C} is independently unsubstituted propyl. In embodiments, R^{8C} is independently unsubstituted isopropyl. In embodiments, R^{8C} is independently unsubstituted tert-butyl.

In embodiments, R^{8D} is independently hydrogen. In embodiments, R^{8D} is independently —CX^{8D}₃. In embodiments, R^{8D} is independently —CHX^{8D}₂. In embodiments, R^{8D} is independently —CH₂X^{8D}. In embodiments, R^{8D} is independently —CN. In embodiments, R^{8D} is independently —COOH. In embodiments, R^{8D} is independently —CONH₂. In embodiments, R^{8D} is independently substituted or unsubstituted alkyl (e.g., C₁-C₈, C₁-C₆, or C₁-C₄). In embodiments, R^{8D} is independently substituted alkyl (e.g., C₁-C₈, C₁-C₆, or C₁-C₄). In embodiments, R^{8D} is independently unsubstituted alkyl (e.g., C₁-C₈, C₁-C₆, or C₁-C₄). In embodiments, R^{8D} is independently substituted or unsubstituted heteroalkyl (e.g., 2 to 8 membered, 2 to 6 membered, or 2 to 4 membered). In embodiments, R^{8D} is independently substituted heteroalkyl (e.g., 2 to 8 membered, 2 to 6 membered, or 2 to 4 membered). In embodiments, R^{8D} is independently unsubstituted heteroalkyl (e.g., 2 to 8 membered, 2 to 6 membered, or 2 to 4 membered). In embodiments, R^{8D} is independently substituted or unsubstituted cycloalkyl (e.g., C₃-C₈, C₃-C₆, or C₅-C₆). In embodiments, R^{8D} is independently substituted cycloalkyl (e.g., C₃-C₈, C₃-C₆, or C₅-C₆). In embodiments, R^{8D} is independently unsubstituted cycloalkyl (e.g., C₃-C₈, C₃-C₆, or C₅-C₆). In embodiments, R^{8D} is independently substituted or unsubstituted heterocycloalkyl (e.g., 3 to 8 membered, 3 to 6 membered, or 5 to 6 membered). In embodiments, R^{8D} is independently substituted heterocycloalkyl (e.g., 3 to 8 membered, 3 to 6 membered, or 5 to 6 membered). In embodiments, R^{8D} is independently unsubstituted heterocycloalkyl (e.g., 3 to 8 membered, 3 to 6 membered, or 5 to 6 membered). In embodiments, R^{8D} is independently substituted or unsubstituted aryl (e.g., C₆-C₁₀, C₁₀, or phenyl). In embodiments, R^{8D} is independently substituted aryl (e.g., C₆-C₁₀, C₁₀, or phenyl). In embodiments, R^{8D} is independently unsubstituted aryl (e.g., C₆-C₁₀, C₁₀, or phenyl). In embodiments, R^{8D} is independently substituted or unsubstituted heteroaryl (e.g., 5 to 10 membered, 5 to 9 membered, or 5 to 6 membered). In embodiments, R^{8D} is independently substituted heteroaryl (e.g., 5 to 10 membered, 5 to 9 membered, or 5 to 6 membered). In embodiments, R^{8D} is independently unsubstituted heteroaryl (e.g., 5 to 10 membered, 5 to 9 membered, or 5 to 6 membered). In embodiments, R^{8D} is independently unsubstituted methyl. In embodiments, R^{8D} is independently unsubstituted ethyl. In embodiments, R^{8D} is independently unsubstituted propyl. In embodiments, R^{8D} is independently unsubstituted isopropyl. In embodiments, R^{8D} is independently unsubstituted tert-butyl.

In embodiments, R⁸ is independently hydrogen, halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, or R⁴⁷-substituted or unsubstituted heteroaryl. In embodiments, R⁸ is independently hydrogen, halogen, —CX⁸₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX⁸₃, —OCHX⁸₂, R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, or R⁴⁷-substituted or unsubstituted heteroaryl.

In embodiments, R⁸ is independently hydrogen, halogen, —CX⁸₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX⁸₃, —OCHX⁸₂, R⁴⁷-substituted or unsubstituted C₁-C₈ alkyl, R⁴⁷-substituted or unsubstituted 2 to 8 membered heteroalkyl, R⁴⁷-substituted or unsubstituted C₃-C₈ cycloalkyl, R⁴⁷-substituted or unsubstituted 3 to 6 membered heterocycloalkyl, R⁴⁷-substituted or unsubstituted phenyl, or R⁴⁷-substituted or unsubstituted 5 to 6 membered heteroaryl. X⁸ is —F, —Cl, —Br, or —I. In embodiments, R⁸ is independently methyl. In embodiments, R⁸ is independently ethyl.

R⁴⁷ is independently oxo, halogen, —CX⁴⁷₃, —CHX⁴⁷₂, —CH₂X⁴⁷, —OCX⁴⁷₃, —OCHX⁴⁷₂, —OCH₂X⁴⁷, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁴⁸-substituted or unsubstituted alkyl (e.g., C₁-C₈, C₁-C₆, or C₁-C₄), R⁴⁸-substituted or unsubstituted heteroalkyl (e.g., 2 to 8 membered, 2 to 6 membered, or 2 to 4 membered), R⁴⁸-substituted or unsubstituted cycloalkyl (e.g., C₃-C₈, C₃-C₆, or C₅-C₆), R⁴⁸-substituted or unsubstituted heterocycloalkyl (e.g., 3 to 8 membered, 3 to 6 membered, or 5 to 6 membered), R⁴⁸-substituted or unsubstituted aryl (e.g., C₆-C₁₀, C₁₀, or phenyl), or R⁴⁸-substituted or unsubstituted heteroaryl (e.g., 5 to 10 membered, 5 to 9 membered, or 5 to 6 membered). X⁴⁷ is —F, —Cl, —Br, or —I.

R⁴⁸ is independently oxo, halogen, —CX⁴⁸₃, —CHX⁴⁸₂, —CH₂X⁴⁸, —OCX⁴⁸₃, —OCHX⁴⁸₂, —OCH₂X⁴⁸, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁴⁹-substituted or unsubstituted alkyl (e.g., C₁-C₈, C₁-C₆, or C₁-C₄), R⁴⁹-substituted or unsubstituted heteroalkyl (e.g., 2 to 8 membered, 2 to 6 membered, or 2 to 4 membered), R⁴⁹-substituted or unsubstituted cycloalkyl (e.g., C₃-C₈, C₃-C₆, or C₅-C₆), R⁴⁹-substituted or unsubstituted heterocycloalkyl (e.g., 3 to 8 membered, 3 to 6 membered, or 5 to 6 membered), R⁴⁹-substituted or unsubstituted aryl (e.g., C₆-C₁₀, C₁₀, or phenyl), or R⁴⁹-substituted or unsubstituted heteroaryl (e.g., 5 to 10 membered, 5 to 9 membered, or 5 to 6 membered). X⁴⁸ is —F, —Cl, —Br, or —I.

In embodiments, R^{8A} is independently hydrogen, —CX^{8A}₃, —CN, —COOH, —CONH₂, —CHX^{8A}₂, —CH₂X^{8A}, R^{47A}-substituted or unsubstituted alkyl, R^{47A}-substituted or unsubstituted heteroalkyl, R^{47A}-substituted or unsubstituted cycloalkyl, R^{47A}-substituted or unsubstituted heterocycloalkyl, R^{47A}-substituted or unsubstituted aryl, or R^{47A}-substituted or unsubstituted heteroaryl. In embodiments, R^{8A} is independently hydrogen, —CX^{8A}₃, —CN, —COOH, —CONH₂, —CHX^{8A}₂, —CH₂X^{8A}, R^{47A}-substituted or unsubstituted C₁-C₈ alkyl, R^{47A}-substituted or unsubstituted 2 to 8 membered heteroalkyl, R^{47A}-substituted or unsubstituted C₃-C₈ cycloalkyl, R^{47A}-substituted or unsubstituted 3 to 6 membered heterocycloalkyl, R^{47A}-substituted or unsubstituted phenyl, or R^{47A}-substituted or unsubstituted 5 to 6 membered heteroaryl. X^{8A} is —F, —Cl, —Br, or —I. In embodiments, R^{8A} is independently hydrogen. In embodiments, R^{8A} is independently methyl. In embodiments, R^{8A} is independently ethyl.

In embodiments, R^{8A} and R^{8B} substituents bonded to the same nitrogen atom may optionally be joined to form a R^{47A}-substituted or unsubstituted heterocycloalkyl or R^{47A}-substituted or unsubstituted heteroaryl. In embodiments, R^{8A} and R^{8B} substituents bonded to the same nitrogen atom may optionally be joined to form a R^{47A}-substituted or unsubstituted 3 to 6 membered heterocycloalkyl or R^{47A}-substituted or unsubstituted 5 to 6 membered heteroaryl.

R^{47A} is independently oxo, halogen, —CX^{47A}₃, —CHX^{47A}₂, —CH₂X^{47A}, —OCX^{47A}₃, —OCHX^{47A}₂, —OCH₂X^{47A}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{48A}-substituted or unsubstituted alkyl (e.g., C₁-C₈, C₁-C₆, or C₁-C₄), R^{48A}-substituted or unsubstituted heteroalkyl (e.g., 2 to 8 membered, 2 to 6 membered, or 2 to 4 membered), R^{48A}-substituted or unsubstituted cycloalkyl (e.g., C₃-C₈, C₃-C₆, or C₅-C₆), R^{48A}-substituted or unsubstituted heterocycloalkyl (e.g., 3 to 8 membered, 3 to 6 membered, or 5 to 6 membered), R^{48A}-substituted or unsubstituted aryl (e.g., C₆-C₁₀, C₁₀, or phenyl), or R^{48A}-substituted or unsubstituted heteroaryl (e.g., 5 to 10 membered, 5 to 9 membered, or 5 to 6 membered). X^{47A} is —F, —Cl, —Br, or —I.

R^{48A} is independently oxo, halogen, —CX^{48A}₃, —CHX^{48A}₂, —CH₂X^{48A}, —OCX^{48A}₃, —OCHX^{48A}₂, —OCH₂X^{48A}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{49A}-substituted or unsubstituted alkyl (e.g., C₁-C₈, C₁-C₆, or C₁-C₄), R^{49A}-substituted or unsubstituted heteroalkyl (e.g., 2 to 8 membered, 2 to 6 membered, or 2 to 4 membered), R^{49A}-substituted or unsubstituted cycloalkyl (e.g., C₃-C₈, C₃-C₆, or C₅-C₆), R^{49A}-substituted or unsubstituted heterocycloalkyl (e.g., 3 to 8 membered, 3 to 6 membered, or 5 to 6 membered), R^{49A}-substituted or unsubstituted aryl (e.g., C₆-C₁₀, C₁₀, or phenyl), or R^{49A}-substituted or unsubstituted heteroaryl (e.g., 5 to 10 membered, 5 to 9 membered, or 5 to 6 membered). X^{48A} is —F, —Cl, —Br, or —I.

In embodiments, R^{8B} is independently hydrogen, —CX^{8B}₃, —CN, —COOH, —CONH₂, —CHX^{8B}₂, —CH₂X^{8B}, R^{47B}-substituted or unsubstituted alkyl, R^{47B}-substituted or unsubstituted heteroalkyl, R^{47B}-substituted or unsubstituted cycloalkyl, R^{47B}-substituted or unsubstituted heterocycloalkyl, R^{47B}-substituted or unsubstituted aryl, or R^{47B}-substituted or unsubstituted heteroaryl. In embodiments, R^{8B} is independently hydrogen, —CX^{8B}₃, —CN, —COOH, —CONH₂, —CHX^{8B}₂, —CH₂X^{8B}, R^{47B}-substituted or unsubstituted C₁-C₈ alkyl, R^{47B}-substituted or unsubstituted 2 to 8 membered heteroalkyl, R^{47B}-substituted or unsubstituted C₃-C₈ cycloalkyl, R^{47B}-substituted or unsubstituted 3 to 6 membered heterocycloalkyl, R^{47B}-substituted or unsubstituted phenyl, or R^{47B}-substituted or unsubstituted 5 to 6 membered heteroaryl. X^{8B} is —F, —Cl, —Br, or —I. In embodiments, R^{8B} is independently hydrogen. In embodiments, R^{8B} is independently methyl. In embodiments, R^{8B} is independently ethyl.

In embodiments, R^{8A} and R^{8B} substituents bonded to the same nitrogen atom may optionally be joined to form a R^{47B}-substituted or unsubstituted heterocycloalkyl or R^{47B}-substituted or unsubstituted heteroaryl. In embodiments, R^{8A} and R^{8B} substituents bonded to the same nitrogen atom may optionally be joined to form a R^{47B}-substituted or unsubstituted 3 to 6 membered heterocycloalkyl or R^{47B}-substituted or unsubstituted 5 to 6 membered heteroaryl.

R^{47B} is independently oxo, halogen, —CX^{47B}₃, —CHX^{47B}₂, —CH₂X^{47B}, —OCX^{47B}₃, —OCHX^{47B}₂, —OCH₂X^{47B}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{48B}-substituted or unsubstituted alkyl (e.g., C₁-C₈, C₁-C₆, or C₁-C₄), R^{48B}-substituted or unsubstituted heteroalkyl (e.g., 2 to 8 membered, 2 to 6 membered, or 2 to 4 membered), R^{48B}-substituted or unsubstituted cycloalkyl (e.g., C₃-C₈, C₃-C₆, or C₅-C₆), R^{48B}-substituted or unsubstituted heterocycloalkyl (e.g., 3 to 8 membered, 3 to 6 membered, or 5 to 6 membered), R^{48B}-substituted or unsubstituted aryl (e.g., C₆-C₁₀, C₁₀, or phenyl), or R^{48B}-substituted or unsubstituted heteroaryl (e.g., 5 to 10 membered, 5 to 9 membered, or 5 to 6 membered). X^{47B} is —F, —Cl, —Br, or —I.

R^{48B} is independently oxo, halogen, —CX^{48B}₃, —CHX^{48B}₂, —CH₂X^{48B}, —OCX^{48B}₃, —OCHX^{48B}₂, —OCH₂X^{48B}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{49B}-substituted or unsubstituted alkyl (e.g., C₁-C₈, C₁-C₆, or C₁-C₄), R^{49B}-substituted or unsubstituted heteroalkyl (e.g., 2 to 8 membered, 2 to 6 membered, or 2 to 4 membered), R^{49B}-substituted or unsubstituted cycloalkyl (e.g., C₃-C₈, C₃-C₆, or C₅-C₆), R^{49B}-substituted or unsubstituted heterocycloalkyl (e.g., 3 to 8 membered, 3 to 6 membered, or 5 to 6 membered), R^{49B}-substituted or unsubstituted aryl (e.g., C₆-C₁₀, C₁₀, or phenyl), or R^{49B}-substituted or unsubstituted heteroaryl (e.g., 5 to 10 membered, 5 to 9 membered, or 5 to 6 membered). X^{48B} is —F, —Cl, —Br, or —I.

In embodiments, R^{8C} is independently hydrogen, —CX^{8C}₃, —CN, —COOH, —CONH₂, —CHX^{8C}₂, —CH₂X^{8C}, R^{47C}-substituted or unsubstituted alkyl, R^{47C}-substituted or unsubstituted heteroalkyl, R^{47C}-substituted or unsubstituted cycloalkyl, R^{47C}-substituted or unsubstituted heterocycloalkyl, R^{47C}-substituted or unsubstituted aryl, or R^{47C}-substituted or unsubstituted heteroaryl. In embodiments, R^{8C} is independently hydrogen, —CX^{8C}₃, —CN, —COOH, —CONH₂, —CHX^{8C}₂, —CH₂X^{8C}, R^{47C}-substituted or unsubstituted C₁-C₈ alkyl, R^{47C}-substituted or unsubstituted 2 to 8 membered heteroalkyl, R^{47C}-substituted or unsubstituted C₃-C₈ cycloalkyl, R^{47C}-substituted or unsubstituted 3 to 6 membered heterocycloalkyl, R^{47C}-substituted or unsubstituted phenyl, or R^{47C}-substituted or unsubstituted 5 to 6 membered heteroaryl. X^{8C} is —F, —Cl, —Br, or —I. In embodiments, R^{8C} is independently hydrogen. In embodiments, R^{8C} is independently methyl. In embodiments, R^{8C} is independently ethyl.

R^{47C} is independently oxo, halogen, —CX^{47C}₃, —CHX^{47C}₂, —CH₂X^{47C}, —OCX^{47C}₃, —OCHX^{47C}₂, —OCH₂X^{47C}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{48C}-substituted or unsubstituted alkyl (e.g., C₁-C₈, C₁-C₆, or C₁-C₄), R^{48C}-substituted or unsubstituted heteroalkyl (e.g., 2 to 8 membered, 2 to 6 membered, or 2 to 4 membered), R^{48C}-substituted or unsubstituted cycloalkyl (e.g., C₃-C₈, C₃-C₆, or C₅-C₆), R^{48C}-substituted or unsubstituted heterocycloalkyl (e.g., 3 to 8 membered, 3 to 6 membered, or 5 to 6 membered), R^{48C}-substituted or unsubstituted aryl (e.g., C₆-C₁₀, C₁₀, or phenyl), or R^{48C}-substituted or unsubstituted heteroaryl (e.g., 5 to 10 membered, 5 to 9 membered, or 5 to 6 membered). X^{47C} is —F, —Cl, —Br, or —I.

R^{48C} is independently oxo, halogen, —CX^{48C}₃, —CHX^{48C}₂, —CH₂X^{48C}, —OCX^{48C}₃, —OCHX^{48C}₂, —OCH₂X^{48C}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{49C}-substituted or unsubstituted alkyl (e.g., C₁-C₈, C₁-C₆, or C₁-C₄), R^{49C}-substituted or unsubstituted heteroalkyl (e.g., 2 to 8 membered, 2 to 6 membered, or 2 to 4 membered), R^{49C}-substituted or unsubstituted cycloalkyl (e.g., C₃-C₈, C₃-C₆, or C₅-C₆), R^{49C}-substituted or unsubstituted heterocycloalkyl (e.g., 3 to 8 membered, 3 to 6 membered, or 5 to 6 membered), R^{49C}-substituted or unsubstituted aryl (e.g., C₆-C₁₀, C₁₀, or phenyl), or R^{49C}-substituted or unsubstituted heteroaryl (e.g., 5 to 10 membered, 5 to 9 membered, or 5 to 6 membered). X^{48C} is —F, —Cl, —Br, or —I.

In embodiments, R^{8D} is independently hydrogen, —CX^{8D}₃, —CN, —COOH, —CONH₂, —CHX^{8D}₂, —CH₂X^{8D}, R^{47D}-substituted or unsubstituted alkyl, R^{47D}-substituted or unsubstituted heteroalkyl, R^{47D}-substituted or unsubstituted cycloalkyl, R^{47D}-substituted or unsubstituted heterocycloalkyl, R^{47D}-substituted or unsubstituted aryl, or R^{47D}-substituted or unsubstituted heteroaryl. In embodiments, R^{8D} is independently hydrogen, —CX^{8D}₃, —CN, —COOH, —CONH₂, —CHX^{8D}₂, —CH₂X^{8D}, R^{47D}-substituted or unsubstituted C₁-C₈ alkyl, R^{47D}-substituted or unsubstituted 2 to 8 membered heteroalkyl, R^{47D}-substituted or unsubstituted C₃-C₈ cycloalkyl, R^{47D}-substituted or unsubstituted 3 to 6 membered heterocycloalkyl, R^{47D}-substituted or unsubstituted phenyl, or R^{47D}-substituted or unsubstituted 5 to 6 membered heteroaryl. X^{8D} is —F, —Cl, —Br, or —I. In embodiments, R^{8D} is independently hydrogen. In embodiments, R^{8D} is independently methyl. In embodiments, R^{8D} is independently ethyl.

R^{47D} is independently oxo, halogen, —CX^{47D}₃, —CHX^{47D}₂, —CH₂X^{47D}, —OCX^{47D}₃, —OCHX^{47D}₂, —OCH₂X^{47D}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{48D}-substituted or unsubstituted alkyl (e.g., C₁-C₈, C₁-C₆, or C₁-C₄), R^{48D}-substituted or unsubstituted heteroalkyl (e.g., 2 to 8 membered, 2 to 6 membered, or 2 to 4 membered), R^{48D}-substituted or unsubstituted cycloalkyl (e.g., C₃-C₈, C₃-C₆, or C₅-C₆), R^{48D}-substituted or unsubstituted heterocycloalkyl (e.g., 3 to 8 membered, 3 to 6 membered, or 5 to 6 membered), R^{48D}-substituted or unsubstituted aryl (e.g., C₆-C₁₀, C₁₀, or phenyl), or R^{48D}-substituted or unsubstituted heteroaryl (e.g., 5 to 10 membered, 5 to 9 membered, or 5 to 6 membered). X^{47D} is —F, —Cl, —Br, or —I.

R^{48D} is independently oxo, halogen, —CX^{48D}₃, —CHX^{48D}₂, —CH₂X^{48D}, —OCX^{48D}₃, —OCHX^{48D}₂, —OCH₂X^{48D}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{49D}-substituted or unsubstituted alkyl (e.g., C₁-C₈, C₁-C₆, or C₁-C₄), R^{49D}-substituted or unsubstituted heteroalkyl (e.g., 2 to 8 membered, 2 to 6 membered, or 2 to 4 membered), R^{49D}-substituted or unsubstituted cycloalkyl (e.g., C₃-C₈, C₃-C₆, or C₅-C₆), R^{49D}-substituted or unsubstituted heterocycloalkyl (e.g., 3 to 8 membered, 3 to 6 membered, or 5 to 6 membered), R^{49D}-substituted or unsubstituted aryl (e.g., C₆-C₁₀, C₁₀, or phenyl), or R^{49D}-substituted or unsubstituted heteroaryl (e.g., 5 to 10 membered, 5 to 9 membered, or 5 to 6 membered). X^{48D} is —F, —Cl, —Br, or —I.

R⁴⁹, R^{49A}, R^{49B}, R^{49C}, and R^{49D} are independently oxo, halogen, —CF₃, —CCl₃, —CBr₃, —CI₃, —CHF₂, —CHCl₂, —CHBr₂, —CH₂F, —CH₂Cl, —CH₂Br, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, R⁹ is an unsubstituted methoxy. In embodiments, R⁹ is —OH. In embodiments, R⁹ is an unsubstituted ethoxy. In embodiments, R⁹ is —N(CH₃)₂. In embodiments, R⁹ is —SH. In embodiments, R⁹ is —SCH₃. In embodiments, R⁹ is —SCH₂CH₃. In embodiments, R⁹ is —NH₂. In embodiments, R⁹ is —NHCH₃. In embodiments, R⁹ is —NHCH₂CH₃. In embodiments, R⁹ is —N(CH₂CH₃)₂. In embodiments, R⁹ is —N(CH₃)(CH₂CH₃). In embodiments, R⁹ is hydrogen.

In embodiments, R⁹ is substituted or unsubstituted C₁-C₆ alkyl. In embodiments, R⁹ is substituted or unsubstituted 2 to 6 membered heteroalkyl. In embodiments, R⁹ is substituted or unsubstituted C₃-C₆ cycloalkyl. In embodiments, R⁹ is substituted or unsubstituted 3 to 6 membered heterocycloalkyl. In embodiments, R⁹ is substituted or unsubstituted phenyl. In embodiments, R⁹ is substituted or unsubstituted 5 to 6 membered heteroaryl. In embodiments, R⁹ is substituted C₁-C₆ alkyl. In embodiments, R⁹ is substituted 2 to 6 membered heteroalkyl. In embodiments, R⁹ is substituted C₃-C₆ cycloalkyl. In embodiments, R⁹ is substituted 3 to 6 membered heterocycloalkyl. In embodiments, R⁹ is substituted phenyl. In embodiments, R⁹ is substituted 5 to 6 membered heteroaryl. In embodiments, R⁹ is an unsubstituted C₁-C₆ alkyl. In embodiments, R⁹ is an unsubstituted 2 to 6 membered heteroalkyl. In embodiments, R⁹ is an unsubstituted C₃-C₆ cycloalkyl. In embodiments, R⁹ is an unsubstituted 3 to 6 membered heterocycloalkyl. In embodiments, R⁹ is an unsubstituted phenyl. In embodiments, R⁹ is an unsubstituted 5 to 6 membered heteroaryl.

In embodiments, R⁹ is substituted or unsubstituted C₁-C₄ alkyl. In embodiments, R⁹ is substituted or unsubstituted C₃-C₅ cycloalkyl. In embodiments, R⁹ is substituted or unsubstituted 3 to 5 membered heterocycloalkyl. In embodiments, R⁹ is substituted or unsubstituted 5 membered heteroaryl. In embodiments, R⁹ is substituted C₁-C₄ alkyl. In embodiments, R⁹ is substituted C₃-C₅ cycloalkyl. In embodiments, R⁹ is substituted 3 to 5 membered heterocycloalkyl. In embodiments, R⁹ is substituted 5 membered heteroaryl. In embodiments, R⁹ is substituted 6 membered heteroaryl. In embodiments, R⁹ is an unsubstituted C₁-C₄ alkyl. In embodiments, R⁹ is an unsubstituted C₃-C₅ cycloalkyl. In embodiments, R⁹ is an unsubstituted 3 to 5 membered heterocycloalkyl. In embodiments, R⁹ is an unsubstituted 5 membered heteroaryl. In embodiments, R⁹ is an unsubstituted 6 membered heteroaryl. In embodiments, R⁹ is substituted C₁-C₃ alkyl. In embodiments, R⁹ is an unsubstituted C₁-C₃ alkyl.

In embodiments, R⁹ is independently hydrogen. In embodiments, R⁹ is independently halogen. In embodiments, R⁹ is independently —CX⁹₃. In embodiments, R⁹ is independently —CHX⁹₂. In embodiments, R⁹ is independently —CH₂X⁹. In embodiments, R⁹ is independently —OCX⁹₃. In embodiments, R⁹ is independently —OCH₂X⁹. In embodiments, R⁹ is independently —OCHX⁹₂. In embodiments, R⁹ is independently —CN. In embodiments, R⁹ is independently —SOₙ₉R^{9D}. In embodiments, R⁹ is independently —SOᵥ₉NR^{9A}R^{9B}. In embodiments, R⁹ is independently —NHC(O)NR^{9A}R^{9B}. In embodiments, R⁹ is independently —N(O)ₘ₉. In embodiments, R⁹ is independently —NR^{9A}R^{9B}. In embodiments, R⁹ is independently —C(O)R^{9C}. In embodiments, R⁹ is independently —C(O)—OR^{9C}. In embodiments, R⁹ is independently —C(O)NR^{9A}R^{9B}. In embodiments, R⁹ is independently —OR^{9D}. In embodiments, R⁹ is independently —NR^{9A}SO₂R^{9D}. In embodiments, R⁹ is independently —NR^{9A}C(O)R^{9C}. In embodiments, R⁹ is independently —NR^{9A}C(O)OR^{9C}. In embodiments, R⁹ is independently —NR^{9A}OR^{9C}. In embodiments, R⁹ is independently —OH. In embodiments, R⁹ is independently —NH₂. In embodiments, R⁹ is independently —COOH. In embodiments, R⁹ is independently —CONH₂. In embodiments, R⁹ is independently —NO₂. In embodiments, R⁹ is independently —SH.

In embodiments, R⁹ is independently substituted or unsubstituted alkyl (e.g., C₁-C₈, C₁-C₆, or C₁-C₄). In embodiments, R⁹ is independently substituted alkyl (e.g., C₁-C₈, C₁-C₆, or C₁-C₄). In embodiments, R⁹ is independently unsubstituted alkyl (e.g., C₁-C₈, C₁-C₆, or C₁-C₄). In embodiments, R⁹ is independently substituted or unsubstituted heteroalkyl (e.g., 2 to 8 membered, 2 to 6 membered, or 2 to 4 membered). In embodiments, R⁹ is independently substituted heteroalkyl (e.g., 2 to 8 membered, 2 to 6 membered, or 2 to 4 membered). In embodiments, R⁹ is independently unsubstituted heteroalkyl (e.g., 2 to 8 membered, 2 to 6 membered, or 2 to 4 membered). In embodiments, R⁹ is independently substituted or unsubstituted cycloalkyl (e.g., C₃-C₈, C₃-C₆, or C₅-C₆). In embodiments, R⁹ is independently substituted cycloalkyl (e.g., C₃-C₈, C₃-C₆, or C₅-C₆). In embodiments, R⁹ is independently unsubstituted cycloalkyl (e.g., C₃-C₈, C₃-C₆, or C₅-C₆). In embodiments, R⁹ is independently substituted or unsubstituted heterocycloalkyl (e.g., 3 to 8 membered, 3 to 6 membered, or 5 to 6 membered). In embodiments, R⁹ is independently substituted heterocycloalkyl (e.g., 3 to 8 membered, 3 to 6 membered, or 5 to 6 membered). In embodiments, R⁹ is independently unsubstituted heterocycloalkyl (e.g., 3 to 8 membered, 3 to 6 membered, or 5 to 6 membered). In embodiments, R⁹ is independently substituted or unsubstituted aryl (e.g., C₆-C₁₀, C₁₀, or phenyl). In embodiments, R⁹ is independently substituted aryl (e.g., C₆-C₁₀, C₁₀, or phenyl). In embodiments, R⁹ is independently unsubstituted aryl (e.g., C₆-C₁₀, C₁₀, or phenyl). In embodiments, R⁹ is independently substituted or unsubstituted heteroaryl (e.g., 5 to 10 membered, 5 to 9 membered, or 5 to 6 membered). In embodiments, R⁹ is independently substituted heteroaryl (e.g., 5 to 10 membered, 5 to 9 membered, or 5 to 6 membered). In embodiments, R⁹ is independently unsubstituted heteroaryl (e.g., 5 to 10 membered, 5 to 9 membered, or 5 to 6 membered).

In embodiments, R^{9A} is independently hydrogen. In embodiments, R^{9A} is independently —CX^{9A}₃. In embodiments, R^{9A} is independently —CHX^{9A}₂. In embodiments, R^{9A} is independently —CH₂X^{9A}. In embodiments, R^{9A} is independently —CN. In embodiments, R^{9A} is independently —COOH. In embodiments, R^{9A} is independently —CONH₂.

In embodiments, R^{9A} is independently substituted or unsubstituted alkyl (e.g., C₁-C₈, C₁-C₆, or C₁-C₄). In embodiments, R^{9A} is independently substituted alkyl (e.g., C₁-C₈, C₁-C₆, or C₁-C₄). In embodiments, R^{9A} is independently unsubstituted alkyl (e.g., C₁-C₈, C₁-C₆, or C₁-C₄). In embodiments, R^{9A} is independently substituted or unsubstituted heteroalkyl (e.g., 2 to 8 membered, 2 to 6 membered, or 2 to 4 membered). In embodiments, R^{9A} is independently substituted heteroalkyl (e.g., 2 to 8 membered, 2 to 6 membered, or 2 to 4 membered). In embodiments, R^{9A} is independently unsubstituted heteroalkyl (e.g., 2 to 8 membered, 2 to 6 membered, or 2 to 4 membered). In embodiments, R^{9A} is independently substituted or unsubstituted cycloalkyl (e.g., C₃-C₈, C₃-C₆, or C₅-C₆). In embodiments, R^{9A} is independently substituted cycloalkyl (e.g., C₃-C₈, C₃-C₆, or C₅-C₆). In embodiments, R^{9A} is independently unsubstituted cycloalkyl (e.g., C₃-C₈, C₃-C₆, or C₅-C₆). In embodiments, R^{9A} is independently substituted or unsubstituted heterocycloalkyl (e.g., 3 to 8 membered, 3 to 6 membered, or 5 to 6 membered). In embodiments, R^{9A} is independently substituted heterocycloalkyl (e.g., 3 to 8 membered, 3 to 6 membered, or 5 to 6 membered). In embodiments, R^{9A} is independently unsubstituted heterocycloalkyl (e.g., 3 to 8 membered, 3 to 6 membered, or 5 to 6 membered). In embodiments, R^{9A} is independently substituted or unsubstituted aryl (e.g., C₆-C₁₀, C₁₀, or phenyl). In embodiments, R^{9A} is independently substituted aryl (e.g., C₆-C₁₀, C₁₀, or phenyl). In embodiments, R^{9A} is independently unsubstituted aryl (e.g., C₆-C₁₀, C₁₀, or phenyl). In embodiments, R^{9A} is independently substituted or unsubstituted heteroaryl (e.g., 5 to 10 membered, 5 to 9 membered, or 5 to 6 membered). In embodiments, R^{9A} is independently substituted heteroaryl (e.g., 5 to 10 membered, 5 to 9 membered, or 5 to 6 membered). In embodiments, R^{9A} is independently unsubstituted heteroaryl (e.g., 5 to 10 membered, 5 to 9 membered, or 5 to 6 membered). In embodiments, R^{9A} is independently unsubstituted methyl. In embodiments, R^{9A} is independently unsubstituted ethyl. In embodiments, R^{9A} is independently unsubstituted propyl. In embodiments, R^{9A} is independently unsubstituted isopropyl. In embodiments, R^{9A} is independently unsubstituted tert-butyl.

In embodiments, R^{9B} is independently hydrogen. In embodiments, R^{9B} is independently —CX^{9B}₃. In embodiments, R^{9B} is independently —CHX^{9B}₂. In embodiments, R^{9B} is independently —CH₂X^{9B}. In embodiments, R^{9B} is independently —CN. In embodiments, R^{9B} is independently —COOH. In embodiments, R^{9B} is independently —CONH₂.

In embodiments, R^{9B} is independently substituted or unsubstituted alkyl (e.g., C₁-C₈, C₁-C₆, or C₁-C₄). In embodiments, R^{9B} is independently substituted alkyl (e.g., C₁-C₈, C₁-C₆, or C₁-C₄). In embodiments, R^{9B} is independently unsubstituted alkyl (e.g., C₁-C₈, C₁-C₆, or C₁-C₄). In embodiments, R^{9B} is independently substituted or unsubstituted heteroalkyl (e.g., 2 to 8 membered, 2 to 6 membered, or 2 to 4 membered). In embodiments, R^{9B} is independently substituted heteroalkyl (e.g., 2 to 8 membered, 2 to 6 membered, or 2 to 4 membered). In embodiments, R^{9B} is independently unsubstituted heteroalkyl (e.g., 2 to 8 membered, 2 to 6 membered, or 2 to 4 membered). In embodiments, R^{9B} is independently substituted or unsubstituted cycloalkyl (e.g., C₃-C₈, C₃-C₆, or C₅-C₆). In embodiments, R^{9B} is independently substituted cycloalkyl (e.g., C₃-C₈, C₃-C₆, or C₅-C₆). In embodiments, R^{9B} is independently unsubstituted cycloalkyl (e.g., C₃-C₈, C₃-C₆, or C₅-C₆). In embodiments, R^{9B} is independently substituted or unsubstituted heterocycloalkyl (e.g., 3 to 8 membered, 3 to 6 membered, or 5 to 6 membered). In embodiments, R^{9B} is independently substituted heterocycloalkyl (e.g., 3 to 8 membered, 3 to 6 membered, or 5 to 6 membered). In embodiments, R^{9B} is independently unsubstituted heterocycloalkyl (e.g., 3 to 8 membered, 3 to 6 membered, or 5 to 6 membered). In embodiments, R^{9B} is independently substituted or unsubstituted aryl (e.g., C₆-C₁₀, C₁₀, or phenyl). In embodiments, R^{9B} is independently substituted aryl (e.g., C₆-C₁₀, C₁₀, or phenyl). In embodiments, R^{9B} is independently unsubstituted aryl (e.g., C₆-C₁₀, C₁₀, or phenyl). In embodiments, R^{9B} is independently substituted or unsubstituted heteroaryl (e.g., 5 to 10 membered, 5 to 9 membered, or 5 to 6 membered). In embodiments, R^{9B} is independently substituted heteroaryl (e.g., 5 to 10 membered, 5 to 9 membered, or 5 to 6 membered). In embodiments, R^{9B} is independently unsubstituted heteroaryl (e.g., 5 to 10 membered, 5 to 9 membered, or 5 to 6 membered). In embodiments, R^{9B} is independently unsubstituted methyl. In embodiments, R^{9B} is independently unsubstituted ethyl. In embodiments, R^{9B} is independently unsubstituted propyl. In embodiments, R^{9B} is independently unsubstituted isopropyl. In embodiments, R^{9B} is independently unsubstituted tert-butyl.

In embodiments, R^{9A} and R^{9B} substituents bonded to the same nitrogen atom may be joined to form a substituted or unsubstituted heterocycloalkyl. In embodiments, R^{9A} and R^{9B} substituents bonded to the same nitrogen atom may be joined to form a substituted or unsubstituted heteroaryl. In embodiments, R^{9A} and R^{9B} substituents bonded to the same nitrogen atom may be joined to form a substituted heterocycloalkyl. In embodiments, R^{9A} and R^{9B} substituents bonded to the same nitrogen atom may be joined to form a substituted heteroaryl. In embodiments, R^{9A} and R^{9B} substituents bonded to the same nitrogen atom may be joined to form an unsubstituted heterocycloalkyl. In embodiments, R^{9A} and R^{9B} substituents bonded to the same nitrogen atom may be joined to form an unsubstituted heteroaryl. In embodiments, R^{9A} and R^{9B} substituents bonded to the same nitrogen atom may be joined to form a substituted or unsubstituted 3 to 8 membered heterocycloalkyl. In embodiments, R^{9A} and R^{9B} substituents bonded to the same nitrogen atom may be joined to form a substituted or unsubstituted 5 to 10 membered heteroaryl. In embodiments, R^{9A} and R^{9B} substituents bonded to the same nitrogen atom may be joined to form a substituted 3 to 8 membered heterocycloalkyl. In embodiments, R^{9A} and R^{9B} substituents bonded to the same nitrogen atom may be joined to form a substituted 5 to 10 membered heteroaryl. In embodiments, R^{9A} and R^{9B} substituents bonded to the same nitrogen atom may be joined to form an unsubstituted 3 to 8 membered heterocycloalkyl. In embodiments, R^{9A} and R^{9B} substituents bonded to the same nitrogen atom may be joined to form an unsubstituted 5 to 10 membered heteroaryl. In embodiments, R^{9A} and R^{9B} substituents bonded to the same nitrogen atom may be joined to form a substituted or unsubstituted 3 to 6 membered heterocycloalkyl. In embodiments, R^{9A} and R^{9B} substituents bonded to the same nitrogen atom may be joined to form a substituted or unsubstituted 5 to 6 membered heteroaryl. In embodiments, R^{9A} and R^{9B} substituents bonded to the same nitrogen atom may be joined to form a substituted 3 to 6 membered heterocycloalkyl. In embodiments, R^{9A} and R^{9B} substituents bonded to the same nitrogen atom may be joined to form a substituted 5 to 6 membered heteroaryl. In embodiments, R^{9A} and R^{9B} substituents bonded to the same nitrogen atom may be joined to form an unsubstituted 3 to 6 membered heterocycloalkyl. In embodiments, R^{9A} and R^{9B} substituents bonded to the same nitrogen atom may be joined to form an unsubstituted 5 to 6 membered heteroaryl.

In embodiments, R^{9C} is independently hydrogen. In embodiments, R⁹ is independently —CX^{9C}₃. In embodiments, R^{9C} is independently —CHX^{9C}₂. In embodiments, R^{9C} is independently —CH₂X^{9C}. In embodiments, R^{9C} is independently —CN. In embodiments, R^{9C} is independently —COOH. In embodiments, R^{9C} is independently —CONH₂. In embodiments, R^{9C} is independently substituted or unsubstituted alkyl (e.g., C₁-C₈, C₁-C₆, or C₁-C₄). In embodiments, R^{9C} is independently substituted alkyl (e.g., C₁-C₈, C₁-C₆, or C₁-C₄). In embodiments, R^{9C} is independently unsubstituted alkyl (e.g., C₁-C₈, C₁-C₆, or C₁-C₄). In embodiments, R^{9C} is independently substituted or unsubstituted heteroalkyl (e.g., 2 to 8 membered, 2 to 6 membered, or 2 to 4 membered). In embodiments, R^{9C} is independently substituted heteroalkyl (e.g., 2 to 8 membered, 2 to 6 membered, or 2 to 4 membered). In embodiments, R^{9C} is independently unsubstituted heteroalkyl (e.g., 2 to 8 membered, 2 to 6 membered, or 2 to 4 membered). In embodiments, R^{9C} is independently substituted or unsubstituted cycloalkyl (e.g., C₃-C₈, C₃-C₆, or C₅-C₆). In embodiments, R^{9C} is independently substituted cycloalkyl (e.g., C₃-C₈, C₃-C₆, or C₅-C₆). In embodiments, R^{9C} is independently unsubstituted cycloalkyl (e.g., C₃-C₈, C₃-C₆, or C₅-C₆). In embodiments, R^{9C} is independently substituted or unsubstituted heterocycloalkyl (e.g., 3 to 8 membered, 3 to 6 membered, or 5 to 6 membered). In embodiments, R^{9C} is independently substituted heterocycloalkyl (e.g., 3 to 8 membered, 3 to 6 membered, or 5 to 6 membered). In embodiments, R^{9C} is independently unsubstituted heterocycloalkyl (e.g., 3 to 8 membered, 3 to 6 membered, or 5 to 6 membered). In embodiments, R^{9C} is independently substituted or unsubstituted aryl (e.g., C₆-C₁₀, C₁₀, or phenyl). In embodiments, R^{9C} is independently substituted aryl (e.g., C₆-C₁₀, C₁₀, or phenyl). In embodiments, R^{9C} is independently unsubstituted aryl (e.g., C₆-C₁₀, C₁₀, or phenyl). In embodiments, R^{9C} is independently substituted or unsubstituted heteroaryl (e.g., 5 to 10 membered, 5 to 9 membered, or 5 to 6 membered). In embodiments, R^{9C} is independently substituted heteroaryl (e.g., 5 to 10 membered, 5 to 9 membered, or 5 to 6 membered). In embodiments, R^{9C} is independently unsubstituted heteroaryl (e.g., 5 to 10 membered, 5 to 9 membered, or 5 to 6 membered). In embodiments, R^{9C} is independently unsubstituted methyl. In embodiments, R^{9C} is independently unsubstituted ethyl. In embodiments, R^{9C} is independently unsubstituted propyl. In embodiments, R^{9C} is independently unsubstituted isopropyl. In embodiments, R^{9C} is independently unsubstituted tert-butyl.

In embodiments, R^{9D} is independently hydrogen. In embodiments, R^{9D} is independently —CX^{9D}₃. In embodiments, R^{9D} is independently —CHX^{9D}₂. In embodiments, R^{9D} is independently —CH₂X^{9D}. In embodiments, R^{9D} is independently —CN. In embodiments, R^{9D} is independently —COOH. In embodiments, R^{9D} is independently —CONH₂.

In embodiments, R^{9D} is independently substituted or unsubstituted alkyl (e.g., C₁-C₈, C₁-C₆, or C₁-C₄). In embodiments, R^{9D} is independently substituted alkyl (e.g., C₁-C₈, C₁-C₆, or C₁-C₄). In embodiments, R^{9D} is independently unsubstituted alkyl (e.g., C₁-C₈, C₁-C₆, or C₁-C₄). In embodiments, R^{9D} is independently substituted or unsubstituted heteroalkyl (e.g., 2 to 8 membered, 2 to 6 membered, or 2 to 4 membered). In embodiments, R^{9D} is independently substituted heteroalkyl (e.g., 2 to 8 membered, 2 to 6 membered, or 2 to 4 membered). In embodiments, R^{9D} is independently unsubstituted heteroalkyl (e.g., 2 to 8 membered, 2 to 6 membered, or 2 to 4 membered). In embodiments, R^{9D} is independently substituted or unsubstituted cycloalkyl (e.g., C₃-C₈, C₃-C₆, or C₅-C₆). In embodiments, R^{9D} is independently substituted cycloalkyl (e.g., C₃-C₈, C₃-C₆, or C₅-C₆). In embodiments, R^{9D} is independently unsubstituted cycloalkyl (e.g., C₃-C₈, C₃-C₆, or C₅-C₆). In embodiments, R^{9D} is independently substituted or unsubstituted heterocycloalkyl (e.g., 3 to 8 membered, 3 to 6 membered, or 5 to 6 membered). In embodiments, R^{9D} is independently substituted heterocycloalkyl (e.g., 3 to 8 membered, 3 to 6 membered, or 5 to 6 membered). In embodiments, R^{9D} is independently unsubstituted heterocycloalkyl (e.g., 3 to 8 membered, 3 to 6 membered, or 5 to 6 membered). In embodiments, R^{9D} is independently substituted or unsubstituted aryl (e.g., C₆-C₁₀, C₁₀, or phenyl). In embodiments, R^{9D} is independently substituted aryl (e.g., C₆-C₁₀, C₁₀, or phenyl). In embodiments, R^{9D} is independently unsubstituted aryl (e.g., C₆-C₁₀, C₁₀, or phenyl). In embodiments, R^{9D} is independently substituted or unsubstituted heteroaryl (e.g., 5 to 10 membered, 5 to 9 membered, or 5 to 6 membered). In embodiments, R^{9D} is independently substituted heteroaryl (e.g., 5 to 10 membered, 5 to 9 membered, or 5 to 6 membered). In embodiments, R^{9D} is independently unsubstituted heteroaryl (e.g., 5 to 10 membered, 5 to 9 membered, or 5 to 6 membered). In embodiments, R^{9D} is independently unsubstituted methyl. In embodiments, R^{9D} is independently unsubstituted ethyl. In embodiments, R^{9D} is independently unsubstituted propyl. In embodiments, R^{9D} is independently unsubstituted isopropyl. In embodiments, R^{9D} is independently unsubstituted tert-butyl.

In embodiments, R⁹ is independently hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)OR^{9C}, —C(O)NR^{9A}R^{9B}, —OR^{9D}, —NR^{9A}SO₂R^{9D}, —NR^{9A}C(O)OR^{9C}, —NR^{9A}OR^{9C}, R⁵³-substituted or unsubstituted alkyl, R⁵³-substituted or unsubstituted heteroalkyl, R⁵³-substituted or unsubstituted cycloalkyl, R⁵³-substituted or unsubstituted heterocycloalkyl, R⁵³-substituted or unsubstituted aryl, or R⁵³-substituted or unsubstituted heteroaryl. In embodiments, R⁹ is independently hydrogen, halogen, —CX⁹₃, —CHX⁹₂, —CH₂X⁹, —OCX⁹₃, —OCH₂X⁹, —OCHX⁹₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁵³-substituted or unsubstituted alkyl, R⁵³-substituted or unsubstituted heteroalkyl, R⁵³-substituted or unsubstituted cycloalkyl, R⁵³-substituted or unsubstituted heterocycloalkyl, R⁵³-substituted or unsubstituted aryl, or R⁵³-substituted or unsubstituted heteroaryl. In embodiments, R⁹ is independently hydrogen, halogen, —CX⁹₃, —CHX⁹₂, —CH₂X⁹, —OCX⁹₃, —OCH₂X⁹, —OCHX⁹₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁵³-substituted or unsubstituted C₁-C₈ alkyl, R⁵³-substituted or unsubstituted 2 to 8 membered heteroalkyl, R⁵³-substituted or unsubstituted C₃-C₈ cycloalkyl, R⁵³-substituted or unsubstituted 3 to 6 membered heterocycloalkyl, R⁵³-substituted or unsubstituted phenyl, or R⁵³-substituted or unsubstituted 5 to 6 membered heteroaryl. X⁹ is —F, —Cl, —Br, or —I. In embodiments, R⁹ is independently hydrogen. In embodiments, R⁹ is independently methyl. In embodiments, R⁹ is independently ethyl.

R⁵³ is independently oxo, halogen, —CX⁵³₃, —CHX⁵³₂, —CH₂X⁵³, —OCX⁵³₃, —OCHX⁵³₂, —OCH₂X⁵³, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁵⁴-substituted or unsubstituted alkyl (e.g., C₁-C₈, C₁-C₆, or C₁-C₄), R⁵⁴-substituted or unsubstituted heteroalkyl (e.g., 2 to 8 membered, 2 to 6 membered, or 2 to 4 membered), R⁵⁴-substituted or unsubstituted cycloalkyl (e.g., C₃-C₈, C₃-C₆, or C₅-C₆), R⁵⁴-substituted or unsubstituted heterocycloalkyl (e.g., 3 to 8 membered, 3 to 6 membered, or 5 to 6 membered), R⁵⁴-substituted or unsubstituted aryl (e.g., C₆-C₁₀, C₁₀, or phenyl), or R⁵⁴-substituted or unsubstituted heteroaryl (e.g., 5 to 10 membered, 5 to 9 membered, or 5 to 6 membered). X⁵³ is —F, —Cl, —Br, or —I.

R⁵⁴ is independently oxo, halogen, —CX⁵⁴₃, —CHX⁵⁴₂, —CH₂X⁵⁴, —OCX⁵⁴₃, —OCHX⁵⁴₂, —OCH₂X⁵⁴, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R⁵⁵-substituted or unsubstituted alkyl (e.g., C₁-C₈, C₁-C₆, or C₁-C₄), R⁵⁵-substituted or unsubstituted heteroalkyl (e.g., 2 to 8 membered, 2 to 6 membered, or 2 to 4 membered), R⁵⁵-substituted or unsubstituted cycloalkyl (e.g., C₃-C₈, C₃-C₆, or C₅-C₆), R⁵⁵-substituted or unsubstituted heterocycloalkyl (e.g., 3 to 8 membered, 3 to 6 membered, or 5 to 6 membered), R⁵⁵-substituted or unsubstituted aryl (e.g., C₆-C₁₀, C₁₀, or phenyl), or R⁵⁵-substituted or unsubstituted heteroaryl (e.g., 5 to 10 membered, 5 to 9 membered, or 5 to 6 membered). X⁵⁴ is —F, —Cl, —Br, or —I.

In embodiments, R^{9A} is independently hydrogen, —CX^{9A}₃, —CN, —COOH, —CONH₂, —CHX^{9A}₂, —CH₂X^{9A}, R^{53A}-substituted or unsubstituted alkyl, R^{53A}-substituted or unsubstituted heteroalkyl, R^{53A}-substituted or unsubstituted cycloalkyl, R^{53A}-substituted or unsubstituted heterocycloalkyl, R^{53A}-substituted or unsubstituted aryl, or R^{53A}-substituted or unsubstituted heteroaryl. In embodiments, R^{9A} is independently hydrogen, —CX^{9A}₃, —CN, —COOH, —CONH₂, —CHX^{9A}₂, —CH₂X^{9A}, R^{53A}-substituted or unsubstituted C₁-C₈ alkyl, R^{53A}-substituted or unsubstituted 2 to 8 membered heteroalkyl, R^{53A}-substituted or unsubstituted C₃-C₈ cycloalkyl, R^{53A}-substituted or unsubstituted 3 to 6 membered heterocycloalkyl, R^{53A}-substituted or unsubstituted phenyl, or R^{53A}-substituted or unsubstituted 5 to 6 membered heteroaryl. X^{9A} is —F, —Cl, —Br, or —I. In embodiments, R^{9A} is independently hydrogen. In embodiments, R^{9A} is independently methyl. In embodiments, R^{9A} is independently ethyl.

In embodiments, R^{9A} and R^{9B} substituents bonded to the same nitrogen atom may optionally be joined to form a R^{53A}-substituted or unsubstituted heterocycloalkyl or R^{53A}-substituted or unsubstituted heteroaryl. In embodiments, R^{9A} and R^{9B} substituents bonded to the same nitrogen atom may optionally be joined to form a R^{53A}-substituted or unsubstituted 3 to 6 membered heterocycloalkyl or R^{53A}-substituted or unsubstituted 5 to 6 membered heteroaryl.

R^{53A} is independently oxo, halogen, —CX^{53A}₃, —CHX^{53A}₂, —CH₂X^{53A}, —OCX^{53A}₃, —OCHX^{53A}₂, —OCH₂X^{53A}, —CN, —OH, —NH₂, —COOH, —CONH₂, —NO₂, —SH, —SO₃H, —SO₄H, —SO₂N⁻H₂, —NHNH₂, —ONH₂, —NHC(O)NHNH₂, —NHC(O)NH₂, —NHSO₂H, —NHC(O)H, —NHC(O)OH, —NHOH, R^{54A}-substituted or unsubstituted alkyl (e.g., C₁-C₈, C₁-C₆, or C₁-C₄), R^{54A}-substituted or unsubstituted heteroalkyl (e.g., 2 to 8 membered, 2 to 6 membered, or 2 to 4 membered), R^{54A}-substituted or unsubstituted cycloalkyl (e.g., C₃-C₈, C₃-C₆, or C₅-C₆), R^{54A}-substituted or unsubstituted heterocycloalkyl (e.g., 3 to 8 membered, 3 to 6 membered, or 5 to 6 membered), R^{54A}-substituted or unsubstituted aryl (e.g., C₆-C₁₀, C₁₀, or phenyl), or R^{54A}-substituted or unsubstituted heteroaryl (e.g., 5 to 10 membered, 5 to 9 membered, or 5 to 6 membered). X^{53A} is —F, —Cl, —Br, or —I.

R^{54A} is independently oxo, halogen, —CX^{54A}₃, —CHX^{54A}₂, —CH₂X^{54A}, —OCX^{54A}₃, —OCHX^{54A}₂, —OCH₂X^{54A}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{55A}-substituted or unsubstituted alkyl (e.g., C₁-C₈, C₁-C₆, or C₁-C₄), R^{55A}-substituted or unsubstituted heteroalkyl (e.g., 2 to 8 membered, 2 to 6 membered, or 2 to 4 membered), R^{55A}-substituted or unsubstituted cycloalkyl (e.g., C₃-C₈, C₃-C₆, or C₅-C₆), R^{55A}-substituted or unsubstituted heterocycloalkyl (e.g., 3 to 8 membered, 3 to 6 membered, or 5 to 6 membered), R^{55A}-substituted or unsubstituted aryl (e.g., C₆-C₁₀, C₁₀, or phenyl), or R^{55A}-substituted or unsubstituted heteroaryl (e.g., 5 to 10 membered, 5 to 9 membered, or 5 to 6 membered). X^{54A} is —F, —Cl, —Br, or —I.

In embodiments, R^{9B} is independently hydrogen, —CX^{9B}₃, —CN, —COOH, —CONH₂, —CHX^{9B}₂, —CH₂X^{9B}, R^{53B}-substituted or unsubstituted alkyl, R^{53B}-substituted or unsubstituted heteroalkyl, R^{53B}-substituted or unsubstituted cycloalkyl, R^{53B}-substituted or unsubstituted heterocycloalkyl, R^{53B}-substituted or unsubstituted aryl, or R^{53B}-substituted or unsubstituted heteroaryl. In embodiments, R^{9B} is independently hydrogen, —CX^{9B}₃, —CN, —COOH, —CONH₂, —CHX^{9B}₂, —CH₂X^{9B}, R^{53B}-substituted or unsubstituted C₁-C₈ alkyl, R^{53B}-substituted or unsubstituted 2 to 8 membered heteroalkyl, R^{53B}-substituted or unsubstituted C₃-C₈ cycloalkyl, R^{53B}-substituted or unsubstituted 3 to 6 membered heterocycloalkyl, R^{53B}-substituted or unsubstituted phenyl, or R^{53B}-substituted or unsubstituted 5 to 6 membered heteroaryl. X^{9B} is —F, —Cl, —Br, or —I. In embodiments, R^{9B} is independently hydrogen. In embodiments, R^{9B} is independently methyl. In embodiments, R^{9B} is independently ethyl.

In embodiments, R^{9A} and R^{9B} substituents bonded to the same nitrogen atom may optionally be joined to form a R^{53B}-substituted or unsubstituted heterocycloalkyl or R^{53B}-substituted or unsubstituted heteroaryl. In embodiments, R^{9A} and R^{9B} substituents bonded to the same nitrogen atom may optionally be joined to form a R^{53B}-substituted or unsubstituted 3 to 6 membered heterocycloalkyl or R^{53B}-substituted or unsubstituted 5 to 6 membered heteroaryl.

R^{53B} is independently oxo, halogen, —CX^{53B}₃, —CHX^{53B}₂, —CH₂X^{53B}, —OCX^{53B}₃, —OCHX^{53B}₂, —OCH₂X^{53B}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{54B}-substituted or unsubstituted alkyl (e.g., C₁-C₈, C₁-C₆, or C₁-C₄), R^{54B}-substituted or unsubstituted heteroalkyl (e.g., 2 to 8 membered, 2 to 6 membered, or 2 to 4 membered), R^{54B}-substituted or unsubstituted cycloalkyl (e.g., C₃-C₈, C₃-C₆, or C₅-C₆), R^{54B}-substituted or unsubstituted heterocycloalkyl (e.g., 3 to 8 membered, 3 to 6 membered, or 5 to 6 membered), R^{54B}-substituted or unsubstituted aryl (e.g., C₆-C₁₀, C₁₀, or phenyl), or R^{54B}-substituted or unsubstituted heteroaryl (e.g., 5 to 10 membered, 5 to 9 membered, or 5 to 6 membered). X^{53B} is —F, —Cl, —Br, or —I.

R^{54B} is independently oxo, halogen, —CX^{54B}₃, —CHX^{54B}₂, —CH₂X^{54B}, —OCX^{54B}₃, —OCHX^{54B}₂, —OCH₂X^{54B}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{55B}-substituted or unsubstituted alkyl (e.g., C₁-C₈, C₁-C₆, or C₁-C₄), R^{55B}-substituted or unsubstituted heteroalkyl (e.g., 2 to 8 membered, 2 to 6 membered, or 2 to 4 membered), R^{55B}-substituted or unsubstituted cycloalkyl (e.g., C₃-C₈, C₃-C₆, or C₅-C₆), R^{55B}-substituted or unsubstituted heterocycloalkyl (e.g., 3 to 8 membered, 3 to 6 membered, or 5 to 6 membered), R^{55B}-substituted or unsubstituted aryl (e.g., C₆-C₁₀, C₁₀, or phenyl), or R^{55B}-substituted or unsubstituted heteroaryl (e.g., 5 to 10 membered, 5 to 9 membered, or 5 to 6 membered). X^{54B} is —F, —Cl, —Br, or —I.

In embodiments, R^{9C} is independently hydrogen, —CX^{9C}₃, —CN, —COOH, —CONH₂, —CHX^{9C}₂, —CH₂X^{9C}, R^{53C}-substituted or unsubstituted alkyl, R^{53C}-substituted or unsubstituted heteroalkyl, R^{53C}-substituted or unsubstituted cycloalkyl, R^{53C}-substituted or unsubstituted heterocycloalkyl, R^{53C}-substituted or unsubstituted aryl, or R^{53C}-substituted or unsubstituted heteroaryl. In embodiments, R^{9C} is independently hydrogen, —CX^{9C}₃, —CN, —COOH, —CONH₂, —CHX^{9C}₂, —CH₂X^{9C}, R^{53C}-substituted or unsubstituted C₁-C₈ alkyl, R^{53C}-substituted or unsubstituted 2 to 8 membered heteroalkyl, R^{53C}-substituted or unsubstituted C₃-C₈ cycloalkyl, R^{53C}-substituted or unsubstituted 3 to 6 membered heterocycloalkyl, R^{53C}-substituted or unsubstituted phenyl, or R^{53C}-substituted or unsubstituted 5 to 6 membered heteroaryl. X^{9C} is —F, —Cl, —Br, or —I. In embodiments, R^{9C} is independently hydrogen. In embodiments, R^{9C} is independently methyl. In embodiments, R^{9C} is independently ethyl.

R^{53C} is independently oxo, halogen, —CX^{53C}₃, —CHX^{53C}₂, —CH₂X^{53C}, —OCX^{53C}₃, —OCHX^{53C}₂, —OCH₂X^{53C}, —CN, —OH, —NH₂, —COOH, —CONH₂, —NO₂, —SH, —SO₃H, —SO₄H, —SO₂N⁻H₂, —ONH₂, —NHC(O)NHNH₂, —NHC(O)NH₂, —NHSO₂H, —NHC(O)H, —NHC(O)OH, —NHOH, R^{54C}-substituted or unsubstituted alkyl (e.g., C₁-C₈, C₁-C₆, or C₁-C₄), R^{54C}-substituted or unsubstituted heteroalkyl (e.g., 2 to 8 membered, 2 to 6 membered, or 2 to 4 membered), R^{54C}-substituted or unsubstituted cycloalkyl (e.g., C₃-C₈, C₃-C₆, or C₅-C₆), R^{54C}-substituted or unsubstituted heterocycloalkyl (e.g., 3 to 8 membered, 3 to 6 membered, or 5 to 6 membered), R^{54C}-substituted or unsubstituted aryl (e.g., C₆-C₁₀, C₁₀, or phenyl), or R^{54C}-substituted or unsubstituted heteroaryl (e.g., 5 to 10 membered, 5 to 9 membered, or 5 to 6 membered). X^{53C} is —F, —Cl, —Br, or —I.

R^{54C} is independently oxo, halogen, —CX^{54C}₃, —CHX^{54C}₂, —CH₂X^{54C}, —OCX^{54C}₃, —OCHX^{54C}₂, —OCH₂X^{54C}, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, R^{55C}-substituted or unsubstituted alkyl (e.g., C₁-C₈, C₁-C₆, or C₁-C₄), R^{55C}-substituted or unsubstituted heteroalkyl (e.g., 2 to 8 membered, 2 to 6 membered, or 2 to 4 membered), R^{55C}-substituted or unsubstituted cycloalkyl (e.g., C₃-C₈, C₃-C₆, or C₅-C₆), R^{55C}-substituted or unsubstituted heterocycloalkyl (e.g., 3 to 8 membered, 3 to 6 membered, or 5 to 6 membered), R^{55C}-substituted or unsubstituted aryl (e.g., C₆-C₁₀, C₁₀, or phenyl), or R^{55C}-substituted or unsubstituted heteroaryl (e.g., 5 to 10 membered, 5 to 9 membered, or 5 to 6 membered). X^{54C} is —F, —Cl, —Br, or —I.

In embodiments, R^{9D} is independently hydrogen, —CX^{9D}₃, —CN, —COOH, —CONH₂, —CHX^{9D}₂, —CH₂X^{9D}, R^{53D}-substituted or unsubstituted alkyl, R^{53D}-substituted or unsubstituted heteroalkyl, R^{53D}-substituted or unsubstituted cycloalkyl, R^{53D}-substituted or unsubstituted heterocycloalkyl, R^{53D}-substituted or unsubstituted aryl, or R^{53D}-substituted or unsubstituted heteroaryl. In embodiments, R^{9D} is independently hydrogen, —CX^{9D}₃, —CN, —COOH, —CONH₂, —CHX^{9D}₂, —CH₂X^{9D}, R^{53D}-substituted or unsubstituted C₁-C₈ alkyl, R^{53D}-substituted or unsubstituted 2 to 8 membered heteroalkyl, R^{53D}-substituted or unsubstituted C₃-C₈ cycloalkyl, R^{53D}-substituted or unsubstituted 3 to 6 membered heterocycloalkyl, R^{53D}-substituted or unsubstituted phenyl, or R^{53D}-substituted or unsubstituted 5 to 6 membered heteroaryl. X^{9D} is —F, —Cl, —Br, or —I. In embodiments, R^{9D} is independently hydrogen. In embodiments, R^{9D} is independently methyl. In embodiments, R^{9D} is independently ethyl.

R^{53D} is independently oxo, halogen, —CX^{53D}₃, —CHX^{53D}₂, —CH₂X^{53D}, —OCX^{53D}₃, —OCHX^{53D}₂, —OCH₂X^{53D}, —CN, —OH, —NH₂, —COOH, —CONH₂, —NO₂, —SH, —SO₃H, —SO₄H, —SO₂NH₂, —NHNH₂, —NHC(O)NHNH₂, —NHC(O)NH₂, —NHSO₂H, —NHC(O)H, —NHC(O)OH, —NHOH, R^{54D}-substituted or unsubstituted alkyl (e.g., C₁-C₈, C₁-C₆, or C₁-C₄), R^{54D}-substituted or unsubstituted heteroalkyl (e.g., 2 to 8 membered, 2 to 6 membered, or 2 to 4 membered), R^{54D}-substituted or unsubstituted cycloalkyl (e.g., C₃-C₈, C₃-C₆, or C₅-C₆), R^{54D}-substituted or unsubstituted heterocycloalkyl (e.g., 3 to 8 membered, 3 to 6 membered, or 5 to 6 membered), R^{54D}-substituted or unsubstituted aryl (e.g., C₆-C₁₀, C₁₀, or phenyl), or R^{54D}-substituted or unsubstituted heteroaryl (e.g., 5 to 10 membered, 5 to 9 membered, or 5 to 6 membered). X^{53D} is —F, —Cl, —Br, or —I.

R^{54D} is independently oxo, halogen, —CX^{54D}₃, —CHX^{54D}₂, —CH₂X^{54D}, —OCX^{54D}₃, —OCHX^{54D}₂, —OCH₂X^{54D}, —CN, —OH, —NH₂, —COOH, —CONH₂, —NO₂, —SH, —SO₃H, —SO₄H, —SO₂NH₂, —NHNH₂, —NHC(O)NHNH₂, —NHC(O)NH₂, —NHSO₂H, —NHC(O)H, —NHC(O)OH, —NHOH, R^{55D}-substituted or unsubstituted alkyl (e.g., C₁-C₈, C₁-C₆, or C₁-C₄), R^{55D}-substituted or unsubstituted heteroalkyl (e.g., 2 to 8 membered, 2 to 6 membered, or 2 to 4 membered), R^{55D}-substituted or unsubstituted cycloalkyl (e.g., C₃-C₈, C₃-C₆, or C₅-C₆), R^{55D}-substituted or unsubstituted heterocycloalkyl (e.g., 3 to 8 membered, 3 to 6 membered, or 5 to 6 membered), R^{55D}-substituted or unsubstituted aryl (e.g., C₆-C₁₀, C₁₀, or phenyl), or R^{55D}-substituted or unsubstituted heteroaryl (e.g., 5 to 10 membered, 5 to 9 membered, or 5 to 6 membered). X^{54D} is —F, —Cl, —Br, or —I.

R⁵⁵, R^{55A}, R^{55B}, R^{55C}, and R^{55D} are independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, E is a covalent cysteine modifier moiety. In embodiments, E is:

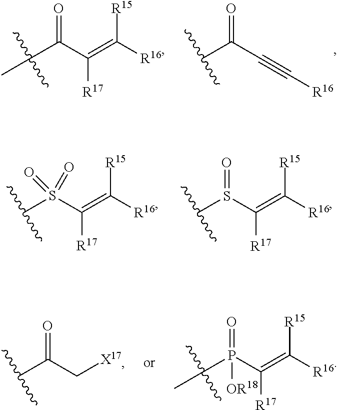

R¹⁵ is independently hydrogen, halogen, CX¹⁵₃, —CHX¹⁵₂, CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;. Each X, X¹⁵, X¹⁶, X¹⁷ and X¹⁸ is independently —F, —Cl, —Br, or —I. n15, n16, n17, v15, v16, and v17, are independently an integer from 0 to 4. m15, m16, and m17 are independently and integer from 1 to 2.

In embodiments, E is:

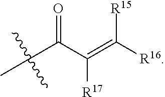

In embodiments, E is:

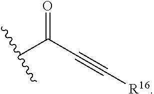

In embodiments, E is:

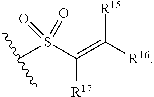

In embodiments, E is:

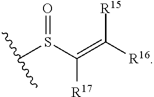

In embodiments, E is:

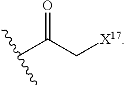

In embodiments, E is:

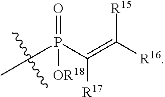

X may independently be —F. X may independently be —Cl. X may independently be —Br. X may independently be —I. X¹⁵ may independently be —F. X¹⁵ may independently be —Cl. X¹⁵ may independently be —Br. X¹⁵ may independently be —I. X¹⁶ may independently be —F. X¹⁶ may independently be —Cl. X¹⁶ may independently be —Br. X¹⁶ may independently be —I. X¹⁷ may independently be —F. X¹⁷ may independently be —Cl. X¹⁷ may independently be —Br. X¹⁷ may independently be —I. X¹⁸ may independently be —F. X¹⁸ may independently be —Cl. X¹⁸ may independently be —Br. X¹⁸ may independently be —I. n15 may independently be 0. n15 may independently be 1. n15 may independently be 2. n15 may independently be 3. n15 may independently be 4. n16 may independently be 0. n16 may independently be 1. n16 may independently be 2. n16 may independently be 3. n16 may independently be 4. n17 may independently be 0. n17 may independently be 1. n17 may independently be 2. n17 may independently be 3. n17 may independently be 4. v15 may independently be 0. v15 may independently be 1. v15 may independently be 2. v15 may independently be 3. v15 may independently be 4. v16 may independently be 0. v16 may independently be 1. v16 may independently be 2. v16 may independently be 3. v16 may independently be 4. m15 may independently be 1. m15 may independently be 2. m16 may independently be 1. m16 may independently be 2. m17 may independently be 1. m17 may independently be 2.

In embodiments, R¹⁵ is hydrogen. In embodiments, R¹⁵ is halogen. In embodiments, R¹⁵ is CX¹⁵₃. In embodiments, R¹⁵ is —CHX¹⁵₂. In embodiments, R¹⁵ is —CH₂X¹⁵. In embodiments, R¹⁵ is —CN. In embodiments, R¹⁵ is —SOₙ₁₅R^{15D}. In embodiments, R¹⁵ is —SOᵥ₁₅NR^{15A}R^{15B}. In embodiments, R^{15 is NHNR}^{15A}R^{15B}. In embodiments, R¹⁵ is —ONR^{15A}R^{15B}. In embodiments, R¹⁵ is —NHC═(O)NHNR^{15A}R^{15B}. In embodiments, R¹⁵ is —NHC(O)NR^{15A}R^{15B}. In embodiments, R¹⁵ is —N(O)ₘ₁₅. In embodiments, R^{15 is —NR}^{15A}R^{15B}. In embodiments, R¹⁵ is —C(O)R^{15C}. In embodiments, R¹⁵ is —C(O)—OR^{15C}. In embodiments, R¹⁵ is —C(O)NR^{15A}R^{15B}. In embodiments, R¹⁵ is —OR^{15D}. In embodiments, R¹⁵ is —NR^{15A}SO₂R^{15D}. In embodiments, R¹⁵ is —NR^{15A}C(O)R^{15C}. In embodiments, R¹⁵ is —NR^{15A}C(O)OR^{15C}. In embodiments, R¹⁵ is —NR^{15A}OR^{15C}. In embodiments, R¹⁵ is —OCX¹⁵₃. In embodiments, R¹⁵ is —OCHX¹⁵₂. In embodiments, R¹⁵ is substituted or unsubstituted alkyl. In embodiments, R¹⁵ is substituted or unsubstituted heteroalkyl. In embodiments, R¹⁵ is substituted or unsubstituted cycloalkyl. In embodiments, R¹⁵ is substituted or unsubstituted heterocycloalkyl. In embodiments, R¹⁵ is substituted or unsubstituted aryl. In embodiments, R¹⁵ is substituted or unsubstituted heteroaryl. In embodiments, R¹⁵ is substituted alkyl. In embodiments, R¹⁵ is substituted heteroalkyl. In embodiments, R¹⁵ is substituted cycloalkyl. In embodiments, R¹⁵ is substituted heterocycloalkyl. In embodiments, R¹⁵ is substituted aryl. In embodiments, R¹⁵ is substituted heteroaryl. In embodiments, R¹⁵ is an unsubstituted alkyl. In embodiments, R¹⁵ is an unsubstituted heteroalkyl. In embodiments, R¹⁵ is an unsubstituted cycloalkyl. In embodiments, R¹⁵ is an unsubstituted heterocycloalkyl. In embodiments, R¹⁵ is an unsubstituted aryl. In embodiments, R¹⁵ is an unsubstituted heteroaryl. In embodiments, R¹⁵ is an unsubstituted methyl. In embodiments, R¹⁵ is an unsubstituted ethyl. In embodiments, R¹⁵ is an unsubstituted propyl. In embodiments, R¹⁵ is an unsubstituted isopropyl. In embodiments, R¹⁵ is an unsubstituted butyl. In embodiments, R¹⁵ is an unsubstituted tert-butyl.

In embodiments, R¹⁵ is substituted or unsubstituted alkyl (e.g., C₁-C₈, C₁-C₆, or C₁-C₄). In embodiments, R¹⁵ is substituted or unsubstituted heteroalkyl (e.g., 2 to 8 membered, 2 to 6 membered, or 2 to 4 membered). In embodiments, R¹⁵ is substituted or unsubstituted cycloalkyl (e.g., C₃-C₈, C₃-C₆, or C₅-C₆). In embodiments, R¹⁵ is substituted or unsubstituted heterocycloalkyl (e.g., 3 to 8 membered, 3 to 6 membered, or 5 to 6 membered). In embodiments, R¹⁵ is substituted or unsubstituted aryl (e.g., C₆-C₁₀, C₁₀, or phenyl). In embodiments, R¹⁵ is substituted or unsubstituted heteroaryl (e.g., 5 to 10 membered, 5 to 9 membered, or 5 to 6 membered).

In embodiments, R¹⁵ is substituted alkyl (e.g., C₁-C₈, C₁-C₆, or C₁-C₄). In embodiments, R¹⁵ is substituted heteroalkyl (e.g., 2 to 8 membered, 2 to 6 membered, or 2 to 4 membered). In embodiments, R¹⁵ is substituted cycloalkyl (e.g., C₃-C₈, C₃-C₆, or C₅-C₆). In embodiments, R¹⁵ is substituted heterocycloalkyl (e.g., 3 to 8 membered, 3 to 6 membered, or 5 to 6 membered). In embodiments, R¹⁵ is substituted aryl (e.g., C₆-C₁₀, C₁₀, or phenyl). In embodiments, R¹⁵ is substituted heteroaryl (e.g., 5 to 10 membered, 5 to 9 membered, or 5 to 6 membered). In embodiments, R¹⁵ is an unsubstituted alkyl (e.g., C₁-C₈, C₁-C₆, or C₁-C₄). In embodiments, R¹⁵ is an unsubstituted heteroalkyl (e.g., 2 to 8 membered, 2 to 6 membered, or 2 to 4 membered). In embodiments, R¹⁵ is an unsubstituted cycloalkyl (e.g., C₃-C₈, C₃-C₆, or C₅-C₆). In embodiments, R¹⁵ is an unsubstituted heterocycloalkyl (e.g., 3 to 8 membered, 3 to 6 membered, or 5 to 6 membered). In embodiments, R¹⁵ is an unsubstituted aryl (e.g., C₆-C₁₀, C₁₀, or phenyl). In embodiments, R¹⁵ is an unsubstituted heteroaryl (e.g., 5 to 10 membered, 5 to 9 membered, or 5 to 6 membered).

In embodiments, R^{15A} is hydrogen. In embodiments, R^{15A} is —CX₃. In embodiments, R^{15A} is —CN. In embodiments, R^{15A} is —COOH. In embodiments, R^{15A} is —CONH₂. In embodiments, R^{15A} is —CHX₂. In embodiments, R^{15A} is —CH₂X. In embodiments, R^{15A} is an unsubstituted methyl. In embodiments, R^{15A} is an unsubstituted ethyl. In embodiments, R^{15A} is an unsubstituted propyl. In embodiments, R^{15A} is an unsubstituted isopropyl. In embodiments, R^{15A} is an unsubstituted butyl. In embodiments, R^{15A} is an unsubstituted tert-butyl.

In embodiments, R^{15B} is hydrogen. In embodiments, R^{15B} is —CX₃. In embodiments, R^{15B} is —CN. In embodiments, R^{15B} is —COOH. In embodiments, R^{15B} is —CONH₂. In embodiments, R^{15B} is —CHX₂. In embodiments, R^{15B} is —CH₂X. In embodiments, R^{15B} is an unsubstituted methyl. In embodiments, R^{15B} is an unsubstituted ethyl. In embodiments, R^{15B} is an unsubstituted propyl. In embodiments, R^{15B} is an unsubstituted isopropyl. In embodiments, R^{15B} is an unsubstituted butyl. In embodiments, R^{15B} is an unsubstituted tert-butyl.

In embodiments, R^{15C} is hydrogen. In embodiments, R^{15C} is —CX₃. In embodiments, R^{15C} is —CN. In embodiments, R^{15C} is —COOH. In embodiments, R^{15C} is —CONH₂. In embodiments, R^{15C} is —CHX₂. In embodiments, R^{15C} is —CH₂X. In embodiments, R^{15C} is an unsubstituted methyl. In embodiments, R^{15C} is an unsubstituted ethyl. In embodiments, R^{15C} is an unsubstituted propyl. In embodiments, R^{15C} is an unsubstituted isopropyl. In embodiments, R^{15C} is an unsubstituted butyl. In embodiments, R^{15C} is an unsubstituted tert-butyl.

In embodiments, R^{15D} is hydrogen. In embodiments, R^{15D} is —CX₃. In embodiments, R^{15D} is —CN. In embodiments, R^{15D} is —COOH. In embodiments, R^{15D} is —CONH₂. In embodiments, R^{15D} is —CHX₂. In embodiments, R^{15D} is —CH₂X. In embodiments, R^{15D} is an unsubstituted methyl. In embodiments, R^{15D} is an unsubstituted ethyl. In embodiments, R^{15D} is an unsubstituted propyl. In embodiments, R^{15D} is an unsubstituted isopropyl. In embodiments, R^{15D} is an unsubstituted butyl. In embodiments, R^{15D} is an unsubstituted tert-butyl.

In embodiments, R¹⁵ is independently hydrogen, oxo, halogen, —CX¹⁵₃, —CHX¹⁵₂, —OCH₂X¹⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁵₃, —OCHX¹⁵₂, R⁷²-substituted or unsubstituted alkyl (e.g., C₁-C₈, C₁-C₆, or C₁-C₄), R⁷²-substituted or unsubstituted heteroalkyl (e.g., 2 to 8 membered, 2 to 6 membered, or 2 to 4 membered), R⁷²-substituted or unsubstituted cycloalkyl (e.g., C₃-C₈, C₃-C₆, or C₅-C₆), R⁷²-substituted or unsubstituted heterocycloalkyl (e.g., 3 to 8 membered, 3 to 6 membered, or 5 to 6 membered), R⁷²-substituted or unsubstituted aryl (e.g., C₆-C₁₀, C₁₀, or phenyl), or R⁷²-substituted or unsubstituted heteroaryl (e.g., 5 to 10 membered, 5 to 9 membered, or 5 to 6 membered). X¹⁵ is halogen. In embodiments, X¹⁵ is F.

R⁷² is independently oxo, halogen, —CX⁷²₃, —CHX⁷²₂, —OCH₂X⁷², —OCHX⁷²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷²₃, —OCHX⁷²₂, R⁷³-substituted or unsubstituted alkyl (e.g., C₁-C₈, C₁-C₆, or C₁-C₄), R⁷³-substituted or unsubstituted heteroalkyl (e.g., 2 to 8 membered, 2 to 6 membered, or 2 to 4 membered), R⁷³-substituted or unsubstituted cycloalkyl (e.g., C₃-C₈, C₃-C₆, or C₅-C₆), R⁷³-substituted or unsubstituted heterocycloalkyl (e.g., 3 to 8 membered, 3 to 6 membered, or 5 to 6 membered), R⁷³-substituted or unsubstituted aryl (e.g., C₆-C₁₀, C₁₀, or phenyl), or R⁷³-substituted or unsubstituted heteroaryl (e.g., 5 to 10 membered, 5 to 9 membered, or 5 to 6 membered). X⁷² is halogen. In embodiments, X⁷² is F.

R⁷³ is independently oxo, halogen, —CX⁷³₃, —CHX⁷³₂, —OCH₂X⁷³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷³₃, —OCHX⁷³₂, R⁷⁴-substituted or unsubstituted alkyl (e.g., C₁-C₈, C₁-C₆, or C₁-C₄), R⁷⁴-substituted or unsubstituted heteroalkyl (e.g., 2 to 8 membered, 2 to 6 membered, or 2 to 4 membered), R⁷⁴-substituted or unsubstituted cycloalkyl (e.g., C₃-C₈, C₃-C₆, or C₅-C₆), R⁷⁴-substituted or unsubstituted heterocycloalkyl (e.g., 3 to 8 membered, 3 to 6 membered, or 5 to 6 membered), R⁷⁴-substituted or unsubstituted aryl (e.g., C₆-C₁₀, C₁₀, or phenyl), or R⁷⁴-substituted or unsubstituted heteroaryl (e.g., 5 to 10 membered, 5 to 9 membered, or 5 to 6 membered). X⁷³ is halogen. In embodiments, X⁷³ is F.

In embodiments, R^{15A} is independently hydrogen, oxo, halogen, —CX^{15A}₃, —CHX^{15A}₂, —OCH₂X^{15A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15A}₃, OCHX^{15A}₂, R^{72A}-substituted or unsubstituted alkyl (e.g., C₁-C₈, C₁-C₆, or C₁-C₄), R^{72A}-substituted or unsubstituted heteroalkyl (e.g., 2 to 8 membered, 2 to 6 membered, or 2 to 4 membered), R^{72A}-substituted or unsubstituted cycloalkyl (e.g., C₃-C₈, C₃-C₆, or C₅-C₆), R^{72A}-substituted or unsubstituted heterocycloalkyl (e.g., 3 to 8 membered, 3 to 6 membered, or 5 to 6 membered), R^{72A}-substituted or unsubstituted aryl (e.g., C₆-C₁₀, C₁₀, or phenyl), or R^{72A}-substituted or unsubstituted heteroaryl (e.g., 5 to 10 membered, 5 to 9 membered, or 5 to 6 membered). X^{15A} is halogen. In embodiments, X^{15A} is F.

R^{72A} is independently oxo, halogen, —CX^{72A}₃, —CHX^{72A}₂, —OCH₂X^{72A}, —OCHX^{72A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72A}₃, —OCHX^{72A}₂, R^{73A}-substituted or unsubstituted alkyl (e.g., C₁-C₈, C₁-C₆, or C₁-C₄), R^{73A}-substituted or unsubstituted heteroalkyl (e.g., 2 to 8 membered, 2 to 6 membered, or 2 to 4 membered), R^{73A}-substituted or unsubstituted cycloalkyl (e.g., C₃-C₈, C₃-C₆, or C₅-C₆), R^{73A}-substituted or unsubstituted heterocycloalkyl (e.g., 3 to 8 membered, 3 to 6 membered, or 5 to 6 membered), R^{73A}-substituted or unsubstituted aryl (e.g., C₆-C₁₀, C₁₀, or phenyl), or R^{73A}-substituted or unsubstituted heteroaryl (e.g., 5 to 10 membered, 5 to 9 membered, or 5 to 6 membered). X^{72A} is halogen. In embodiments, X^{72A} is F.

R^{73A} is independently oxo, halogen, —CX^{73A}₃, —CHX^{73A}₂, —OCH₂X^{73A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73A}₃, —OCHX^{73A}₂, R^{74A}-substituted or unsubstituted alkyl (e.g., C₁-C₈, C₁-C₆, or C₁-C₄), R^{74A}-substituted or unsubstituted heteroalkyl (e.g., 2 to 8 membered, 2 to 6 membered, or 2 to 4 membered), R^{74A}-substituted or unsubstituted cycloalkyl (e.g., C₃-C₈, C₃-C₆, or C₅-C₆), R^{74A}-substituted or unsubstituted heterocycloalkyl (e.g., 3 to 8 membered, 3 to 6 membered, or 5 to 6 membered), R^{74A}-substituted or unsubstituted aryl (e.g., C₆-C₁₀, C₁₀, or phenyl), or R^{74A}-substituted or unsubstituted heteroaryl (e.g., 5 to 10 membered, 5 to 9 membered, or 5 to 6 membered). X^{73A} is halogen. In embodiments, X^{73A} is F.

In embodiments, R^{15B} is independently hydrogen, oxo, halogen, —CX^{15B}₃, —CHX^{15B}₂, —OCH₂X^{15B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15B}₃, —OCHX^{15B}₂, R^{72B}-substituted or unsubstituted alkyl (e.g., C₁-C₈, C₁-C₆, or C₁-C₄), R^{72B}-substituted or unsubstituted heteroalkyl (e.g., 2 to 8 membered, 2 to 6 membered, or 2 to 4 membered), R^{72B}-substituted or unsubstituted cycloalkyl (e.g., C₃-C₈, C₃-C₆, or C₅-C₆), R^{72B}-substituted or unsubstituted heterocycloalkyl (e.g., 3 to 8 membered, 3 to 6 membered, or 5 to 6 membered), R^{72B}-substituted or unsubstituted aryl (e.g., C₆-C₁₀, C₁₀, or phenyl), or R^{72B}-substituted or unsubstituted heteroaryl (e.g., 5 to 10 membered, 5 to 9 membered, or 5 to 6 membered). X^{15B} is halogen. In embodiments, X^{15B} is F.

R^{72B} is independently oxo, halogen, —CX^{72B}₃, —CHX^{72B}₂, —OCH₂X^{72B}, —OCHX^{72B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72B}₃, —OCHX^{72B}₂, R^{73B}-substituted or unsubstituted alkyl (e.g., C₁-C₈, C₁-C₆, or C₁-C₄), R^{73B}-substituted or unsubstituted heteroalkyl (e.g., 2 to 8 membered, 2 to 6 membered, or 2 to 4 membered), R^{73B}-substituted or unsubstituted cycloalkyl (e.g., C₃-C₈, C₃-C₆, or C₅-C₆), R^{73B}-substituted or unsubstituted heterocycloalkyl (e.g., 3 to 8 membered, 3 to 6 membered, or 5 to 6 membered), R^{73B}-substituted or unsubstituted aryl (e.g., C₆-C₁₀, C₁₀, or phenyl), or R^{73B}-substituted or unsubstituted heteroaryl (e.g., 5 to 10 membered, 5 to 9 membered, or 5 to 6 membered). X^{72B} is halogen. In embodiments, X^{72B} is F.

R^{73B} is independently oxo, halogen, —CX^{73B}₃, —CHX^{73B}₂, —OCH₂X^{73B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73B}₃, —OCHX^{73B}₂, R^{74B}-substituted or unsubstituted alkyl (e.g., C₁-C₈, C₁-C₆, or C₁-C₄), R^{74B}-substituted or unsubstituted heteroalkyl (e.g., 2 to 8 membered, 2 to 6 membered, or 2 to 4 membered), R^{74B}-substituted or unsubstituted cycloalkyl (e.g., C₃-C₈, C₃-C₆, or C₅-C₆), R^{74B}-substituted or unsubstituted heterocycloalkyl (e.g., 3 to 8 membered, 3 to 6 membered, or 5 to 6 membered), R^{74B}-substituted or unsubstituted aryl (e.g., C₆-C₁₀, C₁₀, or phenyl), or R^{74B}-substituted or unsubstituted heteroaryl (e.g., 5 to 10 membered, 5 to 9 membered, or 5 to 6 membered). X^{73B} is halogen. In embodiments, X^{73B} is F.

In embodiments, R^{15C} is independently hydrogen, oxo, halogen, —CX^{15C}₃, —CHX^{15C}₂, —OCH₂X^{15C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15C}₃, —OCHX^{15C}₂, R^{72C}-substituted or unsubstituted alkyl (e.g., C₁-C₈, C₁-C₆, or C₁-C₄), R^{72C}-substituted or unsubstituted heteroalkyl (e.g., 2 to 8 membered, 2 to 6 membered, or 2 to 4 membered), R^{72C}-substituted or unsubstituted cycloalkyl (e.g., C₃-C₈, C₃-C₆, or C₅-C₆), R^{72C}-substituted or unsubstituted heterocycloalkyl (e.g., 3 to 8 membered, 3 to 6 membered, or 5 to 6 membered), R^{72C}-substituted or unsubstituted aryl (e.g., C₆-C₁₀, C₁₀, or phenyl), or R^{72C}-substituted or unsubstituted heteroaryl (e.g., 5 to 10 membered, 5 to 9 membered, or 5 to 6 membered). X^{15C} is halogen. In embodiments, X^{15C} is F.

R^{72C} is independently oxo, halogen, —CX^{72C}₃, —CHX^{72C}₂, —OCH₂X^{72C}, —OCHX^{72C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72C}₃, —OCHX^{72C}₂, R^{73C}-substituted or unsubstituted alkyl (e.g., C₁-C₈, C₁-C₆, or C₁-C₄), R^{73C}-substituted or unsubstituted heteroalkyl (e.g., 2 to 8 membered, 2 to 6 membered, or 2 to 4 membered), R^{73C}-substituted or unsubstituted cycloalkyl (e.g., C₃-C₈, C₃-C₆, or C₅-C₆), R^{73C}-substituted or unsubstituted heterocycloalkyl (e.g., 3 to 8 membered, 3 to 6 membered, or 5 to 6 membered), R^{73C}-substituted or unsubstituted aryl (e.g., C₆-C₁₀, C₁₀, or phenyl), or R^{73C}-substituted or unsubstituted heteroaryl (e.g., 5 to 10 membered, 5 to 9 membered, or 5 to 6 membered). X^{72C} is halogen. In embodiments, X^{72C} is F.

R^{73C} is independently oxo, halogen, —CX^{73C}₃, —CHX^{73C}₂, —OCH₂X^{73C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73C}₃, —OCHX^{73C}₂, R^{74C}-substituted or unsubstituted alkyl (e.g., C₁-C₈, C₁-C₆, or C₁-C₄), R^{74C}-substituted or unsubstituted heteroalkyl (e.g., 2 to 8 membered, 2 to 6 membered, or 2 to 4 membered), R^{74C}-substituted or unsubstituted cycloalkyl (e.g., C₃-C₈, C₃-C₆, or C₅-C₆), R^{74C}-substituted or unsubstituted heterocycloalkyl (e.g., 3 to 8 membered, 3 to 6 membered, or 5 to 6 membered), R^{74C}-substituted or unsubstituted aryl (e.g., C₆-C₁₀, C₁₀, or phenyl), or R^{74C}-substituted or unsubstituted heteroaryl (e.g., 5 to 10 membered, 5 to 9 membered, or 5 to 6 membered). X^{73C} is halogen. In embodiments, X^{73C} is F.

In embodiments, R^{15D} is independently hydrogen, oxo, halogen, —CX^{15D}₃, —CHX^{15D}₂, —OCH₂X^{15D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15D}₃, —OCHX^{15D}₂, R^{72D}-substituted or unsubstituted alkyl (e.g., C₁-C₈, C₁-C₆, or C₁-C₄), R^{72D}-substituted or unsubstituted heteroalkyl (e.g., 2 to 8 membered, 2 to 6 membered, or 2 to 4 membered), R^{72D}-substituted or unsubstituted cycloalkyl (e.g., C₃-C₈, C₃-C₆, or C₅-C₆), R^{72D}-substituted or unsubstituted heterocycloalkyl (e.g., 3 to 8 membered, 3 to 6 membered, or 5 to 6 membered), R^{72D}-substituted or unsubstituted aryl (e.g., C₆-C₁₀, C₁₀, or phenyl), or R^{72D}-substituted or unsubstituted heteroaryl (e.g., 5 to 10 membered, 5 to 9 membered, or 5 to 6 membered). X^{15D} is halogen. In embodiments, X^{15D} is F.

R^{72D} is independently oxo, halogen, —CX^{72D}₃, —CHX^{72D}₂, —OCH₂X^{72D}, —OCHX^{72D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72D}₃, —OCHX^{72D}₂, R^{73D}-substituted or unsubstituted alkyl (e.g., C₁-C₈, C₁-C₆, or C₁-C₄), R^{73D}-substituted or unsubstituted heteroalkyl (e.g., 2 to 8 membered, 2 to 6 membered, or 2 to 4 membered), R^{73D}-substituted or unsubstituted cycloalkyl (e.g., C₃-C₈, C₃-C₆, or C₅-C₆), R^{73D}-substituted or unsubstituted heterocycloalkyl (e.g., 3 to 8 membered, 3 to 6 membered, or 5 to 6 membered), R^{73D}-substituted or unsubstituted aryl (e.g., C₆-C₁₀, C₁₀, or phenyl), or R^{73D}-substituted or unsubstituted heteroaryl (e.g., 5 to 10 membered, 5 to 9 membered, or 5 to 6 membered). X^{72D} is halogen. In embodiments, X^{72D} is F.

R^{73D} is independently oxo, halogen, —CX^{73D}₃, —CHX^{73D}₂, —OCH₂X^{73D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73D}₃, —OCHX^{73D}₂, R^{74D}-substituted or unsubstituted alkyl (e.g., C₁-C₈, C₁-C₆, or C₁-C₄), R^{74D}-substituted or unsubstituted heteroalkyl (e.g., 2 to 8 membered, 2 to 6 membered, or 2 to 4 membered), R^{74D}-substituted or unsubstituted cycloalkyl (e.g., C₃-C₈, C₃-C₆, or C₅-C₆), R^{74D}-substituted or unsubstituted heterocycloalkyl (e.g., 3 to 8 membered, 3 to 6 membered, or 5 to 6 membered), R^{74D}-substituted or unsubstituted aryl (e.g., C₆-C₁₀, C₁₀, or phenyl), or R^{74D}-substituted or unsubstituted heteroaryl (e.g., 5 to 10 membered, 5 to 9 membered, or 5 to 6 membered). X^{73D} is halogen. In embodiments, X^{73D} is F.

In embodiments, R¹⁶ is hydrogen. In embodiments, R¹⁶ is halogen. In embodiments, R¹⁶ is CX¹⁶₃. In embodiments, R¹⁶ is —CHX¹⁶₂. In embodiments, R¹⁶ is —CH₂X¹⁶. In embodiments, R¹⁶ is —CN. In embodiments, R¹⁶ is —SOₙ₁₆R^{16D}. In embodiments, R¹⁶ is —SOᵥ₁₆NR^{16A}R^{16B}. In embodiments, R¹⁶ is —NHNR^{16A}R^{16B}. In embodiments, R¹⁶ is —ONR^{16A}R^{16B}. In embodiments, R¹⁶ is —NHC═(O)NHNR^{16A}R^{16B}. In embodiments, R¹⁶ is —NHC(O)NR^{16A}R^{16B}. In embodiments, R¹⁶ is —N(O)ₘ₁₆. In embodiments, R¹⁶ is —NR^{16A}R^{16B}. In embodiments, R¹⁶ is —C(O)R^{16C}. In embodiments, R¹⁶ is —C(O)—OR^{16C}. In embodiments, R¹⁶ is —C(O)NR^{16A}R^{16B}. In embodiments, R¹⁶ is —OR^{16D}. In embodiments, R¹⁶ is —NR^{16A}SO₂R^{16D}. In embodiments, R¹⁶ is —NR^{16A}C(O)R^{16C}. In embodiments, R¹⁶ is —NR^{16A}C(O)OR^{16C}. In embodiments, R¹⁶ is —NR^{16A}OR^{16C}. In embodiments, R¹⁶ is —OCX¹⁶₃. In embodiments, R¹⁶ is —OCHX¹⁶₂. In embodiments, R¹⁶ is substituted or unsubstituted alkyl. In embodiments, R¹⁶ is substituted or unsubstituted heteroalkyl. In embodiments, R¹⁶ is substituted or unsubstituted cycloalkyl. In embodiments, R¹⁶ is substituted or unsubstituted heterocycloalkyl. In embodiments, R¹⁶ is substituted or unsubstituted aryl. In embodiments, R¹⁶ is substituted or unsubstituted heteroaryl. In embodiments, R¹⁶ is substituted alkyl. In embodiments, R¹⁶ is substituted heteroalkyl. In embodiments, R¹⁶ is substituted cycloalkyl. In embodiments, R¹⁶ is substituted heterocycloalkyl. In embodiments, R¹⁶ is substituted aryl. In embodiments, R¹⁶ is substituted heteroaryl. In embodiments, R¹⁶ is an unsubstituted alkyl. In embodiments, R¹⁶ is an unsubstituted heteroalkyl. In embodiments, R¹⁶ is an unsubstituted cycloalkyl. In embodiments, R¹⁶ is an unsubstituted heterocycloalkyl. In embodiments, R¹⁶ is an unsubstituted aryl. In embodiments, R¹⁶ is an unsubstituted heteroaryl. In embodiments, R¹⁶ is an unsubstituted methyl. In embodiments, R¹⁶ is an unsubstituted ethyl. In embodiments, R¹⁶ is an unsubstituted propyl. In embodiments, R¹⁶ is an unsubstituted isopropyl. In embodiments, R¹⁶ is an unsubstituted butyl. In embodiments, R¹⁶ is an unsubstituted tert-butyl.

In embodiments, R¹⁶ is substituted or unsubstituted alkyl (e.g., C₁-C₈, C₁-C₆, or C₁-C₄). In embodiments, R¹⁶ is substituted or unsubstituted heteroalkyl (e.g., 2 to 8 membered, 2 to 6 membered, or 2 to 4 membered). In embodiments, R¹⁶ is substituted or unsubstituted cycloalkyl (e.g., C₃-C₈, C₃-C₆, or C₅-C₆). In embodiments, R¹⁶ is substituted or unsubstituted heterocycloalkyl (e.g., 3 to 8 membered, 3 to 6 membered, or 5 to 6 membered). In embodiments, R¹⁶ is substituted or unsubstituted aryl (e.g., C₆-C₁₀, C₁₀, or phenyl). In embodiments, R¹⁶ is substituted or unsubstituted heteroaryl (e.g., 5 to 10 membered, 5 to 9 membered, or 5 to 6 membered).

In embodiments, R¹⁶ is substituted alkyl (e.g., C₁-C₈, C₁-C₆, or C₁-C₄). In embodiments, R¹⁶ is substituted heteroalkyl (e.g., 2 to 8 membered, 2 to 6 membered, or 2 to 4 membered). In embodiments, R¹⁶ is substituted cycloalkyl (e.g., C₃-C₈, C₃-C₆, or C₅-C₆). In embodiments, R¹⁶ is substituted heterocycloalkyl (e.g., 3 to 8 membered, 3 to 6 membered, or 5 to 6 membered). In embodiments, R¹⁶ is substituted aryl (e.g., C₆-C₁₀, C₁₀, or phenyl). In embodiments, R¹⁶ is substituted heteroaryl (e.g., 5 to 10 membered, 5 to 9 membered, or 5 to 6 membered). In embodiments, R¹⁶ is an unsubstituted alkyl (e.g., C₁-C₈, C₁-C₆, or C₁-C₄). In embodiments, R¹⁶ is an unsubstituted heteroalkyl (e.g., 2 to 8 membered, 2 to 6 membered, or 2 to 4 membered). In embodiments, R¹⁶ is an unsubstituted cycloalkyl (e.g., C₃-C₈, C₃-C₆, or C₅-C₆). In embodiments, R¹⁶ is an unsubstituted heterocycloalkyl (e.g., 3 to 8 membered, 3 to 6 membered, or 5 to 6 membered). In embodiments, R¹⁶ is an unsubstituted aryl (e.g., C₆-C₁₀, C₁₀, or phenyl). In embodiments, R¹⁶ is an unsubstituted heteroaryl (e.g., 5 to 10 membered, 5 to 9 membered, or 5 to 6 membered).

In embodiments, R^{16A} is hydrogen. In embodiments, R^{16A} is —CX₃. In embodiments, R^{16A} is —CN. In embodiments, R^{16A} is —COOH. In embodiments, R^{16A} is —CONH₂. In embodiments, R^{16A} is —CHX₂. In embodiments, R^{16A} is —CH₂X. In embodiments, R^{16A} is an unsubstituted methyl. In embodiments, R^{16A} is an unsubstituted ethyl. In embodiments, R^{16A} is an unsubstituted propyl. In embodiments, R^{16A} is an unsubstituted isopropyl. In embodiments, R^{16A} is an unsubstituted butyl. In embodiments, R^{16A} is an unsubstituted tert-butyl.

In embodiments, R^{16B} is hydrogen. In embodiments, R^{16B} is —CX₃. In embodiments, R^{16B} is —CN. In embodiments, R^{16B} is —COOH. In embodiments, R^{16B} is —CONH₂. In embodiments, R^{16B} is —CHX₂. In embodiments, R^{16B} is —CH₂X. In embodiments, R^{16B} is an unsubstituted methyl. In embodiments, R^{16B} is an unsubstituted ethyl. In embodiments, R^{16B} is an unsubstituted propyl. In embodiments, R^{16B} is an unsubstituted isopropyl. In embodiments, R^{16B} is an unsubstituted butyl. In embodiments, R^{16B} is an unsubstituted tert-butyl.

In embodiments, R^{16C} is hydrogen. In embodiments, R^{16C} is —CX₃. In embodiments, R^{16C} is —CN. In embodiments, R^{16C} is —COOH. In embodiments, R^{16C} is —CONH₂. In embodiments, R^{16C} is —CHX₂. In embodiments, R^{16C} is —CH₂X. In embodiments, R^{16C} is an unsubstituted methyl. In embodiments, R^{16C} is an unsubstituted ethyl. In embodiments, R^{16C} is an unsubstituted propyl. In embodiments, R^{16C} is an unsubstituted isopropyl. In embodiments, R^{16C} is an unsubstituted butyl. In embodiments, R^{16C} is an unsubstituted tert-butyl.

In embodiments, R^{16D} is hydrogen. In embodiments, R^{16D} is —CX₃. In embodiments, R^{16D} is —CN. In embodiments, R^{16D} is —COOH. In embodiments, R^{16D} is —CONH₂. In embodiments, R^{16D} is —CHX₂. In embodiments, R^{16D} is —CH₂X. In embodiments, R^{16D} is an unsubstituted methyl. In embodiments, R^{16D} is an unsubstituted ethyl. In embodiments, R^{16D} is an unsubstituted propyl. In embodiments, R^{16D} is an unsubstituted isopropyl. In embodiments, R^{16D} is an unsubstituted butyl. In embodiments, R^{16D} is an unsubstituted tert-butyl.

In embodiments, R¹⁶ is independently hydrogen, oxo, halogen, —CX¹⁶₃, —CHX¹⁶₂, —OCH₂X¹⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁶₃, —OCHX¹⁶₂, R⁷⁵-substituted or unsubstituted alkyl (e.g., C₁-C₈, C₁-C₆, or C₁-C₄), R⁷⁵-substituted or unsubstituted heteroalkyl (e.g., 2 to 8 membered, 2 to 6 membered, or 2 to 4 membered), R⁷⁵-substituted or unsubstituted cycloalkyl (e.g., C₃-C₈, C₃-C₆, or C₅-C₆), R⁷⁵-substituted or unsubstituted heterocycloalkyl (e.g., 3 to 8 membered, 3 to 6 membered, or 5 to 6 membered), R⁷⁵-substituted or unsubstituted aryl (e.g., C₆-C₁₀, C₁₀, or phenyl), or R⁷⁵-substituted or unsubstituted heteroaryl (e.g., 5 to 10 membered, 5 to 9 membered, or 5 to 6 membered). X¹⁶ is halogen. In embodiments, X¹⁶ is F.

R⁷⁵ is independently oxo, halogen, —CX⁷⁵₃, —CHX⁷⁵₂, —OCH₂X⁷⁵, —OCHX⁷⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁵₃, —OCHX⁷⁵₂, R⁷⁶-substituted or unsubstituted alkyl (e.g., C₁-C₈, C₁-C₆, or C,-C₄), R⁷⁶-substituted or unsubstituted heteroalkyl (e.g., 2 to 8 membered, 2 to 6 membered, or 2 to 4 membered), R⁷⁶-substituted or unsubstituted cycloalkyl (e.g., C₃-C₈, C₃-C₆, or C₅-C₆), R⁷⁶-substituted or unsubstituted heterocycloalkyl (e.g., 3 to 8 membered, 3 to 6 membered, or 5 to 6 membered), R⁷⁶-substituted or unsubstituted aryl (e.g., C₆-C₁₀, C,₁₀, or phenyl), or R⁷⁶-substituted or unsubstituted heteroaryl (e.g., 5 to 10 membered, 5 to 9 membered, or 5 to 6 membered). X⁷⁵ is halogen. In embodiments, X⁷⁵ is F.

R⁷⁶ is independently oxo, halogen, —CX⁷⁶₃, —CHX⁷⁶₂, —OCH₂X⁷⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁶₃, —OCHX⁷⁶₂, R⁷⁷-substituted or unsubstituted alkyl (e.g., C₁-C₈, C₁-C₆, or C₁-C₄), R⁷⁷-substituted or unsubstituted heteroalkyl (e.g., 2 to 8 membered, 2 to 6 membered, or 2 to 4 membered), R⁷⁷-substituted or unsubstituted cycloalkyl (e.g., C₃-C₈, C₃-C₆, or C₅-C₆), R⁷⁷-substituted or unsubstituted heterocycloalkyl (e.g., 3 to 8 membered, 3 to 6 membered, or 5 to 6 membered), R⁷⁷-substituted or unsubstituted aryl (e.g., C₆-C₁₀, C₁₀, or phenyl), or R⁷⁷-substituted or unsubstituted heteroaryl (e.g., 5 to 10 membered, 5 to 9 membered, or 5 to 6 membered). X⁷⁶ is halogen. In embodiments, X⁷⁶ is F.

In embodiments, R^{16A} is independently hydrogen, oxo, halogen, —CX^{16A}₃, —CHX^{16A}₂, —OCH₂X^{16A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16A}₃, —OCHX^{16A}₂, R^{75A}-substituted or unsubstituted alkyl (e.g., C₁-C₈, C₁-C₆, or C₁-C₄), R^{75A}-substituted or unsubstituted heteroalkyl (e.g., 2 to 8 membered, 2 to 6 membered, or 2 to 4 membered), R^{75A}-substituted or unsubstituted cycloalkyl (e.g., C₃-C₈, C₃-C₆, or C₅-C₆), R^{75A}-substituted or unsubstituted heterocycloalkyl (e.g., 3 to 8 membered, 3 to 6 membered, or 5 to 6 membered), R^{75A}-substituted or unsubstituted aryl (e.g., C₆-C₁₀, C₁₀, or phenyl), or R^{75A}-substituted or unsubstituted heteroaryl (e.g., 5 to 10 membered, 5 to 9 membered, or 5 to 6 membered). X^{16A} is halogen. In embodiments, X^{16A} is F.

R^{75A} is independently oxo, halogen, —CX^{75A}₃, —CHX^{75A}₂, —OCH₂X^{75A}, —OCHX^{75A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75A}₃, —OCHX^{75A}₂, R^{76A}-substituted or unsubstituted alkyl (e.g., C₁-C₈, C₁-C₆, or C₁-C₄), R^{76A}-substituted or unsubstituted heteroalkyl (e.g., 2 to 8 membered, 2 to 6 membered, or 2 to 4 membered), R^{76A}-substituted or unsubstituted cycloalkyl (e.g., C₃-C₈, C₃-C₆, or C₅-C₆), R^{76A}-substituted or unsubstituted heterocycloalkyl (e.g., 3 to 8 membered, 3 to 6 membered, or 5 to 6 membered), R^{76A}-substituted or unsubstituted aryl (e.g., C₆-C₁₀, C₁₀, or phenyl), or R^{76A}-substituted or unsubstituted heteroaryl (e.g., 5 to 10 membered, 5 to 9 membered, or 5 to 6 membered). X^{75A} is halogen. In embodiments, X^{75A} is F.

R^{76A} is independently oxo, halogen, —CX^{76A}₃, —CHX^{76A}₂, —OCH₂X^{76A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76A}₃, —OCHX^{76A}₂, R^{77A}-substituted or unsubstituted alkyl (e.g., C₁-C₈, C₁-C₆, or C₁-C₄), R^{77A}-substituted or unsubstituted heteroalkyl (e.g., 2 to 8 membered, 2 to 6 membered, or 2 to 4 membered), R^{77A}-substituted or unsubstituted cycloalkyl (e.g., C₃-C₈, C₃-C₆, or C₅-C₆), R^{77A}-substituted or unsubstituted heterocycloalkyl (e.g., 3 to 8 membered, 3 to 6 membered, or 5 to 6 membered), R^{77A}-substituted or unsubstituted aryl (e.g., C₆-C₁₀, C₁₀, or phenyl), or R^{77A}-substituted or unsubstituted heteroaryl (e.g., 5 to 10 membered, 5 to 9 membered, or 5 to 6 membered). X^{76A} is halogen. In embodiments, X^{76A} is F.

In embodiments, R^{16B} is independently hydrogen, oxo, halogen, —CX^{16B}₃, —CHX^{16B}₂, —OCH₂X^{16B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16B}₃, —OCHX^{16B}₂, R^{75B}-substituted or unsubstituted alkyl (e.g., C₁-C₈, C₁-C₆, or C₁-C₄), R^{75B}-substituted or unsubstituted heteroalkyl (e.g., 2 to 8 membered, 2 to 6 membered, or 2 to 4 membered), R^{75B}-substituted or unsubstituted cycloalkyl (e.g., C₃-C₈, C₃-C₆, or C₅-C₆), R^{75B}-substituted or unsubstituted heterocycloalkyl (e.g., 3 to 8 membered, 3 to 6 membered, or 5 to 6 membered), R^{75B}-substituted or unsubstituted aryl (e.g., C₆-C₁₀, C₁₀, or phenyl), or R^{75B}-substituted or unsubstituted heteroaryl (e.g., 5 to 10 membered, 5 to 9 membered, or 5 to 6 membered). X^{16B} is halogen. In embodiments, X^{16B} is F.

R^{75B} is independently oxo, halogen, —CX^{75B}₃, —CHX^{75B}₂, —OCH₂X^{75B}, —OCHX^{75B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75B}₃, —OCHX^{75B}₂, R^{76B}-substituted or unsubstituted alkyl (e.g., C₁-C₈, C₁-C₆, or C₁-C₄), R^{76B}-substituted or unsubstituted heteroalkyl (e.g., 2 to 8 membered, 2 to 6 membered, or 2 to 4 membered), R^{76B}-substituted or unsubstituted cycloalkyl (e.g., C₃-C₈, C₃-C₆, or C₅-C₆), R^{76B}-substituted or unsubstituted heterocycloalkyl (e.g., 3 to 8 membered, 3 to 6 membered, or 5 to 6 membered), R^{76B}-substituted or unsubstituted aryl (e.g., C₆-C₁₀, C₁₀, or phenyl), or R^{76B}-substituted or unsubstituted heteroaryl (e.g., 5 to 10 membered, 5 to 9 membered, or 5 to 6 membered). X^{75B} is halogen. In embodiments, X^{75B} is F.

R^{76B} is independently oxo, halogen, —CX OCH₂X^{76B}, —CHX^{76B}₂, —OCH₂X^{76B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76B}₃, —OCHX^{76B}₂, R^{77B}-substituted or unsubstituted alkyl (e.g., C₁-C₈, C₁-C₆, or C₁-C₄), R^{77B}-substituted or unsubstituted heteroalkyl (e.g., 2 to 8 membered, 2 to 6 membered, or 2 to 4 membered), R^{77B}-substituted or unsubstituted cycloalkyl (e.g., C₃-C₈, C₃-C₆, or C₅-C₆), R^{77B}-substituted or unsubstituted heterocycloalkyl (e.g., 3 to 8 membered, 3 to 6 membered, or 5 to 6 membered), R^{77B}-substituted or unsubstituted aryl (e.g., C₆-C₁₀, C₁₀, or phenyl), or R^{77B}-substituted or unsubstituted heteroaryl (e.g., 5 to 10 membered, 5 to 9 membered, or 5 to 6 membered). X^{76B} is halogen. In embodiments, X^{76B} is F.

In embodiments, R^{16C} is independently hydrogen, oxo, halogen, —CX^{16C}₃, —CHX^{16C}₂, —OCH₂X^{16C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16C}₃, —OCHX^{16C}₂, R^{75C}-substituted or unsubstituted alkyl (e.g., C₁-C₈, C₁-C₆, or C₁-C₄), R^{75C}-substituted or unsubstituted heteroalkyl (e.g., 2 to 8 membered, 2 to 6 membered, or 2 to 4 membered), R^{75C}-substituted or unsubstituted cycloalkyl (e.g., C₃-C₈, C₃-C₆, or C₅-C₆), R^{75C}-substituted or unsubstituted heterocycloalkyl (e.g., 3 to 8 membered, 3 to 6 membered, or 5 to 6 membered), R^{75C}-substituted or unsubstituted aryl (e.g., C₆-C₁₀, C₁₀, or phenyl), or R^{75C}-substituted or unsubstituted heteroaryl (e.g., 5 to 10 membered, 5 to 9 membered, or 5 to 6 membered). X^{16C} is halogen. In embodiments, X^{16C} is F.

R^{75C} is independently oxo, halogen, —CX^{75C}₃, —CHX^{75C}₂, —OCH₂X^{75C}, —OCHX^{75C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75C}₃, —OCHX^{75C}₂, R^{76C}-substituted or unsubstituted alkyl (e.g., C₁-C₈, C₁-C₆, or C₁-C₄), R^{76C}-substituted or unsubstituted heteroalkyl (e.g., 2 to 8 membered, 2 to 6 membered, or 2 to 4 membered), R^{76C}-substituted or unsubstituted cycloalkyl (e.g., C₃-C₈, C₃-C₆, or C₅-C₆), R^{76C}-substituted or unsubstituted heterocycloalkyl (e.g., 3 to 8 membered, 3 to 6 membered, or 5 to 6 membered), R^{76C}-substituted or unsubstituted aryl (e.g., C₆-C₁₀, C₁₀, or phenyl), or R^{76C}-substituted or unsubstituted heteroaryl (e.g., 5 to 10 membered, 5 to 9 membered, or 5 to 6 membered). X^{75C} is halogen. In embodiments, X^{75C} is F.

R^{76C} is independently oxo, halogen, —CX^{76C}₃, —CHX^{76C}₂, —OCH₂X^{76C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76C}₃, —OCHX^{76C}₂, R^{77C}-substituted or unsubstituted alkyl (e.g., C₁-C₈, C₁-C₆, or C₁-C₄), R^{77C}-substituted or unsubstituted heteroalkyl (e.g., 2 to 8 membered, 2 to 6 membered, or 2 to 4 membered), R^{77C}-substituted or unsubstituted cycloalkyl (e.g., C₃-C₈, C₃-C₆, or C₅-C₆), R^{77C}-substituted or unsubstituted heterocycloalkyl (e.g., 3 to 8 membered, 3 to 6 membered, or 5 to 6 membered), R^{77C}-substituted or unsubstituted aryl (e.g., C₆-C₁₀, C₁₀, or phenyl), or R^{77C}-substituted or unsubstituted heteroaryl (e.g., 5 to 10 membered, 5 to 9 membered, or 5 to 6 membered). X^{76C} is halogen. In embodiments, X^{76C} is F.

In embodiments, R^{16D} is independently hydrogen, oxo, halogen, —CX^{16D}₃, —CHX^{16D}₂, —OCH₂X^{16D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16D}₃, —OCHX^{16D}₂, R^{75D}-substituted or unsubstituted alkyl (e.g., C₁-C₈, C₁-C₆, or C₁-C₄), R^{75D}-substituted or unsubstituted heteroalkyl (e.g., 2 to 8 membered, 2 to 6 membered, or 2 to 4 membered), R^{75D}-substituted or unsubstituted cycloalkyl (e.g., C₃-C₈, C₃-C₆, or C₅-C₆), R^{75D}-substituted or unsubstituted heterocycloalkyl (e.g., 3 to 8 membered, 3 to 6 membered, or 5 to 6 membered), R^{75D}-substituted or unsubstituted aryl (e.g., C₆-C₁₀, C₁₀, or phenyl), or R^{75D}-substituted or unsubstituted heteroaryl (e.g., 5 to 10 membered, 5 to 9 membered, or 5 to 6 membered). X^{16D} is halogen. In embodiments, X^{16D} is F.

R^{75D} is independently oxo, halogen, —CX^{75D}₃, —CHX^{75D}₂, —OCH₂X^{75D}, —OCHX^{75D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75D}₃, —OCHX^{75D}₂, R^{76D}-substituted or unsubstituted alkyl (e.g., C₁-C₈, C₁-C₆, or C₁-C₄), R^{76D}-substituted or unsubstituted heteroalkyl (e.g., 2 to 8 membered, 2 to 6 membered, or 2 to 4 membered), R^{76D}-substituted or unsubstituted cycloalkyl (e.g., C₃-C₈, C₃-C₆, or C₅-C₆), R^{76D}-substituted or unsubstituted heterocycloalkyl (e.g., 3 to 8 membered, 3 to 6 membered, or 5 to 6 membered), R^{76D}-substituted or unsubstituted aryl (e.g., C₆-C₁₀, C₁₀, or phenyl), or R^{76D}-substituted or unsubstituted heteroaryl (e.g., 5 to 10 membered, 5 to 9 membered, or 5 to 6 membered). X^{75D} is halogen. In embodiments, X^{75D} is F.

R^{76D} is independently oxo, halogen, —CX^{76D}₃, —CHX^{76D}₂, —OCH₂X^{76D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76D}₃, —OCHX^{76D}₂, R^{77D}-substituted or unsubstituted alkyl (e.g., C₁-C₈, C₁-C₆, or C₁-C₄), R^{77D}-substituted or unsubstituted heteroalkyl (e.g., 2 to 8 membered, 2 to 6 membered, or 2 to 4 membered), R^{77D}-substituted or unsubstituted cycloalkyl (e.g., C₃-C₈, C₃-C₆, or C₅-C₆), R^{77D}-substituted or unsubstituted heterocycloalkyl (e.g., 3 to 8 membered, 3 to 6 membered, or 5 to 6 membered), R^{77D}-substituted or unsubstituted aryl (e.g., C₆-C₁₀, C₁₀, or phenyl), or R^{77D}-substituted or unsubstituted heteroaryl (e.g., 5 to 10 membered, 5 to 9 membered, or 5 to 6 membered). X^{76D} is halogen. In embodiments, X^{76D} is F.

In embodiments, R¹⁷ is hydrogen. In embodiments, R¹⁷ is halogen. In embodiments, R¹⁷ is CX¹⁷₃. In embodiments, R¹⁷ is —CHX¹⁷₂. In embodiments, R¹⁷ is —CH₂X¹⁷. In embodiments, R¹⁷ is —CN. In embodiments, R¹⁷ is —SOₙ₁₇R^{17D}. In embodiments, R¹⁷ is —SOᵥ₁₇NR^{17A}R^{17B}. In embodiments, R¹⁷ is —NHNR^{17A}R^{17B}. In embodiments, R¹⁷ is —ONR^{17A}R^{17B}. In embodiments, R¹⁷ is —NHC═(O)NHNR^{17A}R^{17B}. In embodiments, R¹⁷ is —NHC(O)NR^{17A}R^{17B}. In embodiments, R¹⁷ is —N(O)ₘ₁₇. In embodiments, R¹⁷ is —NR^{17A}R^{17B}. In embodiments, R¹⁷ is —C(O)R^{17C}. In embodiments, R¹⁷ is —C(O)—OR^{17C}. In embodiments, R¹⁷ is —C(O)NR^{17A}R^{17B}. In embodiments, R¹⁷ is —OR^{17D}. In embodiments, R¹⁷ is —NR^{17A}SO₂R^{17D}. In embodiments, R¹⁷ is —NR^{17A}C(O)R^{17C}. In embodiments, R¹⁷ is —NR^{17A}C(O)OR^{17C}. In embodiments R¹⁷ is —NR^{17A}OR^{17C}. In embodiments, R¹⁷ is —OCX¹⁷₃. In embodiments, R¹⁷ is —OCHX¹⁷₂. In embodiments, R¹⁷ is substituted or unsubstituted alkyl. In embodiments, R¹⁷ is substituted or unsubstituted heteroalkyl. In embodiments, R¹⁷ is substituted or unsubstituted cycloalkyl. In embodiments, R¹⁷ is substituted or unsubstituted heterocycloalkyl. In embodiments, R¹⁷ is substituted or unsubstituted aryl. In embodiments, R¹⁷ is substituted or unsubstituted heteroaryl. In embodiments, R¹⁷ is substituted alkyl. In embodiments, R¹⁷ is substituted heteroalkyl. In embodiments, R¹⁷ is substituted cycloalkyl. In embodiments, R¹⁷ is substituted heterocycloalkyl. In embodiments, R¹⁷ is substituted aryl. In embodiments, R¹⁷ is substituted heteroaryl. In embodiments, R¹⁷ is an unsubstituted alkyl. In embodiments, R¹⁷ is an unsubstituted heteroalkyl. In embodiments, R¹⁷ is an unsubstituted cycloalkyl. In embodiments, R¹⁷ is an unsubstituted heterocycloalkyl. In embodiments, R¹⁷ is an unsubstituted aryl. In embodiments, R¹⁷ is an unsubstituted heteroaryl. In embodiments, R¹⁷ is an unsubstituted methyl. In embodiments, R¹⁷ is an unsubstituted ethyl. In embodiments, R¹⁷ is an unsubstituted propyl. In embodiments, R¹⁷ is an unsubstituted isopropyl. In embodiments, R¹⁷ is an unsubstituted butyl. In embodiments, R¹⁷ is an unsubstituted tert-butyl.

In embodiments, R^{17A} is hydrogen. In embodiments, R^{17A} is —CX₃. In embodiments, R^{17A} is —CN. In embodiments, R^{17A} is —COOH. In embodiments, R^{17A} is —CONH₂. In embodiments, R^{17A} is —CHX₂. In embodiments, R^{17A} is —CH₂X. In embodiments, R^{17A} is an unsubstituted methyl. In embodiments, R^{17A} is an unsubstituted ethyl. In embodiments, R^{17A} is an unsubstituted propyl. In embodiments, R^{17A} is an unsubstituted isopropyl. In embodiments, R^{17A} is an unsubstituted butyl. In embodiments, R^{17A} is an unsubstituted tert-butyl.

In embodiments, R¹⁷ is substituted or unsubstituted alkyl (e.g., C₁-C₈, C₁-C₆, or C₁-C₄). In embodiments, R¹⁷ is substituted or unsubstituted heteroalkyl (e.g., 2 to 8 membered, 2 to 6 membered, or 2 to 4 membered). In embodiments, R¹⁷ is substituted or unsubstituted cycloalkyl (e.g., C₃-C₈, C₃-C₆, or C₅-C₆). In embodiments, R¹⁷ is substituted or unsubstituted heterocycloalkyl (e.g., 3 to 8 membered, 3 to 6 membered, or 5 to 6 membered). In embodiments, R¹⁷ is substituted or unsubstituted aryl (e.g., C₆-C₁₀, C₁₀, or phenyl). In embodiments, R¹⁷ is substituted or unsubstituted heteroaryl (e.g., 5 to 10 membered, 5 to 9 membered, or 5 to 6 membered).

In embodiments, R¹⁷ is substituted alkyl (e.g., C₁-C₈, C₁-C₆, or C₁-C₄). In embodiments, R¹⁷ is substituted heteroalkyl (e.g., 2 to 8 membered, 2 to 6 membered, or 2 to 4 membered). In embodiments, R¹⁷ is substituted cycloalkyl (e.g., C₃-C₈, C₃-C₆, or C₅-C₆). In embodiments, R¹⁷ is substituted heterocycloalkyl (e.g., 3 to 8 membered, 3 to 6 membered, or 5 to 6 membered). In embodiments, R¹⁷ is substituted aryl (e.g., C₆-C₁₀, C₁₀, or phenyl). In embodiments, R¹⁷ is substituted heteroaryl (e.g., 5 to 10 membered, 5 to 9 membered, or 5 to 6 membered). In embodiments, R¹⁷ is an unsubstituted alkyl (e.g., C₁-C₈, C₁-C₆, or C₁-C₄). In embodiments, R¹⁷ is an unsubstituted heteroalkyl (e.g., 2 to 8 membered, 2 to 6 membered, or 2 to 4 membered). In embodiments, R¹⁷ is an unsubstituted cycloalkyl (e.g., C₃-C₈, C₃-C₆, or C₅-C₆). In embodiments, R¹⁷ is an unsubstituted heterocycloalkyl (e.g., 3 to 8 membered, 3 to 6 membered, or 5 to 6 membered). In embodiments, R¹⁷ is an unsubstituted aryl (e.g., C₆-C₁₀, C₁₀, or phenyl). In embodiments, R¹⁷ is an unsubstituted heteroaryl (e.g., 5 to 10 membered, 5 to 9 membered, or 5 to 6 membered).

In embodiments, R^{17B} is hydrogen. In embodiments, R^{17B} is —CX₃. In embodiments, R^{17B} is —CN. In embodiments, R^{17B} is —COOH. In embodiments, R^{17B} is —CONH₂. In embodiments, R^{17B} is —CHX₂. In embodiments, R^{17B} is —CH₂X. In embodiments, R^{17B} is an unsubstituted methyl. In embodiments, R^{17B} is an unsubstituted ethyl. In embodiments, R^{17B} is an unsubstituted propyl. In embodiments, R^{17B} is an unsubstituted isopropyl. In embodiments, R^{17B} is an unsubstituted butyl. In embodiments, R^{17B} is an unsubstituted tert-butyl.

In embodiments, R^{17C} is hydrogen. In embodiments, R^{17C} is —CX₃. In embodiments, R^{17C} is —CN. In embodiments, R^{17C} is —COOH. In embodiments, R^{17C} is —CONH₂. In embodiments, R^{17C} is —CHX₂. In embodiments, R^{17C} is —CH₂X. In embodiments, R^{17C} is an unsubstituted methyl. In embodiments, R^{17C} is an unsubstituted ethyl. In embodiments, R^{17C} is an unsubstituted propyl. In embodiments, R^{17C} is an unsubstituted isopropyl. In embodiments, R^{17C} is an unsubstituted butyl. In embodiments, R^{17C} is an unsubstituted tert-butyl.

In embodiments, R^{17D} is hydrogen. In embodiments, R^{17D} is —CX₃. In embodiments, R^{17D} is —CN. In embodiments, R^{17D} is —COOH. In embodiments, R^{17D} is —CONH₂. In embodiments, R^{17D} is —CHX₂. In embodiments, R^{17D} is —CH₂X. In embodiments, R^{17D} is an unsubstituted methyl. In embodiments, R^{17D} is an unsubstituted ethyl. In embodiments, R^{17D} is an unsubstituted propyl. In embodiments, R^{17D} is an unsubstituted isopropyl. In embodiments, R^{17D} is an unsubstituted butyl. In embodiments, R^{17D} is an unsubstituted tert-butyl.

In embodiments, R¹⁷ is independently hydrogen, oxo, halogen, —CX¹⁷₃, —CHX¹⁷₂, —OCH₂X¹⁷, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁷₃, —OCHX¹⁷₂, R⁷⁸-substituted or unsubstituted alkyl (e.g., C₁-C₈, C₁-C₆, or C₁-C₄), R⁷⁸-substituted or unsubstituted heteroalkyl (e.g., 2 to 8 membered, 2 to 6 membered, or 2 to 4 membered), R⁷⁸-substituted or unsubstituted cycloalkyl (e.g., C₃-C₈, C₃-C₆, or C₅-C₆), R⁷⁸-substituted or unsubstituted heterocycloalkyl (e.g., 3 to 8 membered, 3 to 6 membered, or 5 to 6 membered), R⁷⁸-substituted or unsubstituted aryl (e.g., C₆-C₁₀, C₁₀, or phenyl), or R⁷⁸-substituted or unsubstituted heteroaryl (e.g., 5 to 10 membered, 5 to 9 membered, or 5 to 6 membered). X¹⁷ is halogen. In embodiments, X¹⁷ is F.

R⁷⁸ is independently oxo, halogen, —CX⁷⁸₃, —CHX⁷⁸₂, —OCH₂X⁷⁸, —OCHX⁷⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁸₃, —OCHX⁷⁸₂, R⁷⁹-substituted or unsubstituted alkyl (e.g., C₁-C₈, C₁-C₆, or C₁-C₄), R⁷⁹-substituted or unsubstituted heteroalkyl (e.g., 2 to 8 membered, 2 to 6 membered, or 2 to 4 membered), R⁷⁹-substituted or unsubstituted cycloalkyl (e.g., C₃-C₈, C₃-C₆, or C₅-C₆), R⁷⁹-substituted or unsubstituted heterocycloalkyl (e.g., 3 to 8 membered, 3 to 6 membered, or 5 to 6 membered), R⁷⁹-substituted or unsubstituted aryl (e.g., C₆-C₁₀, C₁₀, or phenyl), or R⁷⁹-substituted or unsubstituted heteroaryl (e.g., 5 to 10 membered, 5 to 9 membered, or 5 to 6 membered). X⁷⁸ is halogen. In embodiments, X⁷⁸ is F.

R⁷⁹ is independently oxo, halogen, —CX⁷⁹₃, —CHX⁷⁹₂, —OCH₂X⁷⁹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁹₃, —OCHX⁷⁹₂, R⁸⁰-substituted or unsubstituted alkyl (e.g., C₁-C₈, C₁-C₆, or C₁-C₄), R⁸⁰-substituted or unsubstituted heteroalkyl (e.g., 2 to 8 membered, 2 to 6 membered, or 2 to 4 membered), R⁸⁰-substituted or unsubstituted cycloalkyl (e.g., C₃-C₈, C₃-C₆, or C₅-C₆), R⁸⁰-substituted or unsubstituted heterocycloalkyl (e.g., 3 to 8 membered, 3 to 6 membered, or 5 to 6 membered), R⁸⁰-substituted or unsubstituted aryl (e.g., C₆-C₁₀, C₁₀, or phenyl), or R⁸⁰-substituted or unsubstituted heteroaryl (e.g., 5 to 10 membered, 5 to 9 membered, or 5 to 6 membered). x⁷⁹ is halogen. In embodiments, X⁷⁹ is F.

In embodiments, R^{17A} is independently hydrogen, oxo, halogen, —CX^{17A}₃, —CHX^{17A}₂, —OCH₂X^{17A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17A}₃, —OCHX^{17A}₂, R^{78A}-substituted or unsubstituted alkyl (e.g., C₁-C₈, C₁-C₆, or C₁-C₄), R^{78A}-substituted or unsubstituted heteroalkyl (e.g., 2 to 8 membered, 2 to 6 membered, or 2 to 4 membered), R^{78A}-substituted or unsubstituted cycloalkyl (e.g., C₃-C₈, C₃-C₆, or C₅-C₆), R^{78A}-substituted or unsubstituted heterocycloalkyl (e.g., 3 to 8 membered, 3 to 6 membered, or 5 to 6 membered), R^{78A}-substituted or unsubstituted aryl (e.g., C₆-C₁₀, C₁₀, or phenyl), or R^{78A}-substituted or unsubstituted heteroaryl (e.g., 5 to 10 membered, 5 to 9 membered, or 5 to 6 membered). X^{17A} is halogen. In embodiments, X^{17A} is F.

R^{78A} is independently oxo, halogen, —CX^{78A}₃, —CHX^{78A}₂, —OCH₂X^{78A}, —OCHX^{78A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78A}₃, —OCHX^{78A}₂, R^{79A}-substituted or unsubstituted alkyl (e.g., C₁-C₈, C₁-C₆, or C₁-C₄), R^{79A}-substituted or unsubstituted heteroalkyl (e.g., 2 to 8 membered, 2 to 6 membered, or 2 to 4 membered), R^{79A}-substituted or unsubstituted cycloalkyl (e.g., C₃-C₈, C₃-C₆, or C₅-C₆), R^{79A}-substituted or unsubstituted heterocycloalkyl (e.g., 3 to 8 membered, 3 to 6 membered, or 5 to 6 membered), R^{79A}-substituted or unsubstituted aryl (e.g., C₆-C₁₀, C₁₀, or phenyl), or R^{79A}-substituted or unsubstituted heteroaryl (e.g., 5 to 10 membered, 5 to 9 membered, or 5 to 6 membered). X^{78A} is halogen. In embodiments, X^{78A} is F.

R^{79A} is independently oxo, halogen, —CX^{79A}₃, —CHX^{79A}₂, —OCH₂X^{79A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79A}₃, —OCHX^{79A}₂, R^{80A}-substituted or unsubstituted alkyl (e.g., C₁-C₈, C₁-C₆, or C₁-C₄), R^{80A}-substituted or unsubstituted heteroalkyl (e.g., 2 to 8 membered, 2 to 6 membered, or 2 to 4 membered), R^{80A}-substituted or unsubstituted cycloalkyl (e.g., C₃-C₈, C₃-C₆, or C₅-C₆), R^{80A}-substituted or unsubstituted heterocycloalkyl (e.g., 3 to 8 membered, 3 to 6 membered, or 5 to 6 membered), R^{80A}-substituted or unsubstituted aryl (e.g., C₆-C₁₀, C₁₀, or phenyl), or R^{80A}-substituted or unsubstituted heteroaryl (e.g., 5 to 10 membered, 5 to 9 membered, or 5 to 6 membered). X^{79A} is halogen. In embodiments, X^{79A} is F.

In embodiments, R^{17B} is independently hydrogen, oxo, halogen, —CX^{17B}₃, —CHX^{17B}₂, —OCH₂X^{17B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17B}₃, —OCHX^{17B}₂, R^{78B}-substituted or unsubstituted alkyl (e.g., C₁-C₈, C₁-C₆, or C₁-C₄), R^{78B}-substituted or unsubstituted heteroalkyl (e.g., 2 to 8 membered, 2 to 6 membered, or 2 to 4 membered), R^{78B}-substituted or unsubstituted cycloalkyl (e.g., C₃-C₈, C₃-C₆, or C₅-C₆), R^{78B}-substituted or unsubstituted heterocycloalkyl (e.g., 3 to 8 membered, 3 to 6 membered, or 5 to 6 membered), R^{78B}-substituted or unsubstituted aryl (e.g., C₆-C₁₀, C₁₀, or phenyl), or R^{78B}-substituted or unsubstituted heteroaryl (e.g., 5 to 10 membered, 5 to 9 membered, or 5 to 6 membered). X^{17B} is halogen. In embodiments, X^{17B} is F.

R^{78B} is independently oxo, halogen, —CX^{78B}₃, —CHX^{78B}₂, —OCH₂X^{78B}, —OCHX^{78B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78B}₃, —OCHX^{78B}₂, R^{79B}-substituted or unsubstituted alkyl (e.g., C₁-C₈, C₁-C₆, or C₁-C₄), R^{79B}-substituted or unsubstituted heteroalkyl (e.g., 2 to 8 membered, 2 to 6 membered, or 2 to 4 membered), R^{79B}-substituted or unsubstituted cycloalkyl (e.g., C₃-C₈, C₃-C₆, or C₅-C₆), R^{79B}-substituted or unsubstituted heterocycloalkyl (e.g., 3 to 8 membered, 3 to 6 membered, or 5 to 6 membered), R^{79B}-substituted or unsubstituted aryl (e.g., C₆-C₁₀, C₁₀, or phenyl), or R^{79B}-substituted or unsubstituted heteroaryl (e.g., 5 to 10 membered, 5 to 9 membered, or 5 to 6 membered). X^{78B} is halogen. In embodiments, X^{78B} is F.

R^{79B} is independently oxo, halogen, —CX^{79B}₃, —CHX^{79B}₂, —OCH₂X^{79B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79B}₃, —OCHX^{79B}₂, R^{80B}-substituted or unsubstituted alkyl (e.g., C₁-C₈, C₁-C₆, or C₁-C₄), R^{80B}-substituted or unsubstituted heteroalkyl (e.g., 2 to 8 membered, 2 to 6 membered, or 2 to 4 membered), R^{80B}-substituted or unsubstituted cycloalkyl (e.g., C₃-C₈, C₃-C₆, or C₅-C₆), R^{80B}-substituted or unsubstituted heterocycloalkyl (e.g., 3 to 8 membered, 3 to 6 membered, or 5 to 6 membered), R^{80B}-substituted or unsubstituted aryl (e.g., C₆-C₁₀, C₁₀, or phenyl), or R^{80B}-substituted or unsubstituted heteroaryl (e.g., 5 to 10 membered, 5 to 9 membered, or 5 to 6 membered). X^{79B} is halogen. In embodiments, X^{79B} is F.

In embodiments, R^{17C} is independently hydrogen, oxo, halogen, —CX^{17C}₃, —CHX^{17C}₂, —OCH₂X^{17C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17C}₃, —OCHX^{17C}₂, R^{78C}-substituted or unsubstituted alkyl (e.g., C₁-C₈, C₁-C₆, or C₁-C₄), R^{78C}-substituted or unsubstituted heteroalkyl (e.g., 2 to 8 membered, 2 to 6 membered, or 2 to 4 membered), R^{78C}-substituted or unsubstituted cycloalkyl (e.g., C₃-C₈, C₃-C₆, or C₅-C₆), R^{78C}-substituted or unsubstituted heterocycloalkyl (e.g., 3 to 8 membered, 3 to 6 membered, or 5 to 6 membered), R^{78C}-substituted or unsubstituted aryl (e.g., C₆-C₁₀, C₁₀, or phenyl), or R^{78C}-substituted or unsubstituted heteroaryl (e.g., 5 to 10 membered, 5 to 9 membered, or 5 to 6 membered). X^{17C} is halogen. In embodiments, X^{17C} is F.

R^{78C} is independently oxo, halogen, —CX^{78C}₃, —CHX^{78C}₂, —OCH₂X^{78C}, —OCHX^{78C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78C}₃, —OCHX^{78C}₂, R^{79C}-substituted or unsubstituted alkyl (e.g., C₁-C₈, C₁-C₆, or C₁-C₄), R^{79C}-substituted or unsubstituted heteroalkyl (e.g., 2 to 8 membered, 2 to 6 membered, or 2 to 4 membered), R^{79C}-substituted or unsubstituted cycloalkyl (e.g., C₃-C₈, C₃-C₆, or C₅-C₆), R^{79C}-substituted or unsubstituted heterocycloalkyl (e.g., 3 to 8 membered, 3 to 6 membered, or 5 to 6 membered), R^{79C}-substituted or unsubstituted aryl (e.g., C₆-C₁₀, C₁₀, or phenyl), or R^{79C}-substituted or unsubstituted heteroaryl (e.g., 5 to 10 membered, 5 to 9 membered, or 5 to 6 membered). X^{78C} is halogen. In embodiments, X^{78C} is F.

R^{79C} is independently oxo, halogen, —CX^{79C}₃, —CHX^{79C}₂, —OCH₂X^{79C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79C}₃, —OCHX^{79C}₂, R^{80C}-substituted or unsubstituted alkyl (e.g., C₁-C₈, C₁-C₆, or C₁-C₄), R^{80C}-substituted or unsubstituted heteroalkyl (e.g., 2 to 8 membered, 2 to 6 membered, or 2 to 4 membered), R^{80C}-substituted or unsubstituted cycloalkyl (e.g., C₃-C₈, C₃-C₆, or C₅-C₆), R^{80C}-substituted or unsubstituted heterocycloalkyl (e.g., 3 to 8 membered, 3 to 6 membered, or 5 to 6 membered), R^{80C}-substituted or unsubstituted aryl (e.g., C₆-C₁₀, C₁₀, or phenyl), or R^{80C}-substituted or unsubstituted heteroaryl (e.g., 5 to 10 membered, 5 to 9 membered, or 5 to 6 membered). X^{79C} is halogen. In embodiments, X^{79C} is F.

In embodiments, R^{17D} is independently hydrogen, oxo, halogen, —CX^{17D}₃, —CHX^{17D}₂, —OCH₂X^{17D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17D}₃, —OCHX^{17D}₂, R^{78D}-substituted or unsubstituted alkyl (e.g., C₁-C₈, C₁-C₆, or C₁-C₄), R^{78D}-substituted or unsubstituted heteroalkyl (e.g., 2 to 8 membered, 2 to 6 membered, or 2 to 4 membered), R^{78D}-substituted or unsubstituted cycloalkyl (e.g., C₃-C₈, C₃-C₆, or C₅-C₆), R^{78D}-substituted or unsubstituted heterocycloalkyl (e.g., 3 to 8 membered, 3 to 6 membered, or 5 to 6 membered), R^{78D}-substituted or unsubstituted aryl (e.g., C₆-C₁₀, C₁₀, or phenyl), or R^{78D}-substituted or unsubstituted heteroaryl (e.g., 5 to 10 membered, 5 to 9 membered, or 5 to 6 membered). X^{17D} is halogen. In embodiments, X^{17D} is F.

R^{78D} is independently oxo, halogen, —CX^{78D}₃, —CHX^{78D}₂, —OCH₂X^{78D}, —OCHX^{78D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78D}₃, —OCHX^{78D}₂, R^{79D}-substituted or unsubstituted alkyl (e.g., C₁-C₈, C₁-C₆, or C₁-C₄), R^{79D}-substituted or unsubstituted heteroalkyl (e.g., 2 to 8 membered, 2 to 6 membered, or 2 to 4 membered), R^{79D}-substituted or unsubstituted cycloalkyl (e.g., C₃-C₈, C₃-C₆, or C₅-C₆), R^{79D}-substituted or unsubstituted heterocycloalkyl (e.g., 3 to 8 membered, 3 to 6 membered, or 5 to 6 membered), R^{79D}-substituted or unsubstituted aryl (e.g., C₆-C₁₀, C₁₀, or phenyl), or R^{79D}-substituted or unsubstituted heteroaryl (e.g., 5 to 10 membered, 5 to 9 membered, or 5 to 6 membered). X^{78D} is halogen. In embodiments, X^{78D} is F.

R^{79D} is independently oxo, halogen, —CX^{79D}₃, —CHX^{79D}₂, —OCH₂X^{79D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79D}₃, —OCHX^{79D}₂, R^{80D}-substituted or unsubstituted alkyl (e.g., C₁-C₈, C₁-C₆, or C₁-C₄), R^{80D}-substituted or unsubstituted heteroalkyl (e.g., 2 to 8 membered, 2 to 6 membered, or 2 to 4 membered), R^{80D}-substituted or unsubstituted cycloalkyl (e.g., C₃-C₈, C₃-C₆, or C₅-C₆), R^{80D}-substituted or unsubstituted heterocycloalkyl (e.g., 3 to 8 membered, 3 to 6 membered, or 5 to 6 membered), R^{80D}-substituted or unsubstituted aryl (e.g., C₆-C₁₀, C₁₀, or phenyl), or R^{80D}-substituted or unsubstituted heteroaryl (e.g., 5 to 10 membered, 5 to 9 membered, or 5 to 6 membered). X^{79D} is halogen. In embodiments, X^{79D} is F.

In embodiments, R¹⁸ is hydrogen. In embodiments, R¹⁸ is halogen. In embodiments, R¹⁸ is CX¹⁸₃. In embodiments, R¹⁸ is —CHX¹⁸₂. In embodiments, R¹⁸ is —CH₂X¹⁸. In embodiments, R¹⁸ is —CN. In embodiments, R¹⁸ is —SOₙ₁₈R^{18D}. In embodiments, R¹⁸ is —SOᵥ₁₈NR^{18A}R^{18B}. In embodiments, R¹⁸ is —NHNR^{18A}R^{18B}. In embodiments, R¹⁸ is —ONR^{18A}R^{18B}. In embodiments, R¹⁸ is —NHC═(O)NHNR^{18A}R^{18B}. In embodiments, R¹⁸ is —NHC(O)NR^{18A}R^{18B}. In embodiments, R¹⁸ is —N(O)ₘ₁₈. In embodiments, R¹⁸ is —NR^{18A}R^{18B}. In embodiments, R¹⁸ is —C(O)R^{18C}. In embodiments, R¹⁸ is —C(O)—OR^{18C}. In embodiments, R¹⁸ is —C(O)NR^{18A}R^{18B}. In embodiments, R¹⁸ is —OR^{18D}. In embodiments, R¹⁸ is —NR^{18A}SO₂R^{18D}. In embodiments, R¹⁸ is —NR^{18A}C(O)R^{18C}. In embodiments, R¹⁸ is —NR^{18A}C(O)OR^{18C}. In embodiments R¹⁸ is —NR^{18A}OR^{18C}. In embodiments, R¹⁸ is —OCX¹⁸₃. In embodiments, R¹⁸ is —OCHX¹⁸₂. In embodiments, R¹⁸ is substituted or unsubstituted alkyl. In embodiments, R¹⁸ is substituted or unsubstituted heteroalkyl. In embodiments, R¹⁸ is substituted or unsubstituted cycloalkyl. In embodiments, R¹⁸ is substituted or unsubstituted heterocycloalkyl. In embodiments, R¹⁸ is substituted or unsubstituted aryl. In embodiments, R¹⁸ is substituted or unsubstituted heteroaryl. In embodiments, R¹⁸ is substituted alkyl. In embodiments, R¹⁸ is substituted heteroalkyl. In embodiments, R¹⁸ is substituted cycloalkyl. In embodiments, R¹⁸ is substituted heterocycloalkyl. In embodiments, R¹⁸ is substituted aryl. In embodiments, R¹⁸ is substituted heteroaryl. In embodiments, R¹⁸ is an unsubstituted alkyl. In embodiments, R¹⁸ is an unsubstituted heteroalkyl. In embodiments, R¹⁸ is an unsubstituted cycloalkyl. In embodiments, R¹⁸ is an unsubstituted heterocycloalkyl. In embodiments, R¹⁸ is an unsubstituted aryl. In embodiments, R¹⁸ is an unsubstituted heteroaryl. In embodiments, R¹⁸ is an unsubstituted methyl. In embodiments, R¹⁸ is an unsubstituted ethyl. In embodiments, R¹⁸ is an unsubstituted propyl. In embodiments, R¹⁸ is an unsubstituted isopropyl. In embodiments, R¹⁸ is an unsubstituted butyl. In embodiments, R¹⁸ is an unsubstituted tert-butyl.

In embodiments, R¹⁷ is substituted or unsubstituted alkyl (e.g., C₁-C₈, C₁-C₆, or C₁-C₄). In embodiments, R¹⁷ is substituted or unsubstituted heteroalkyl (e.g., 2 to 8 membered, 2 to 6 membered, or 2 to 4 membered). In embodiments, R¹⁷ is substituted or unsubstituted cycloalkyl (e.g., C₃-C₈, C₃-C₆, or C₅-C₆). In embodiments, R¹⁷ is substituted or unsubstituted heterocycloalkyl (e.g., 3 to 8 membered, 3 to 6 membered, or 5 to 6 membered). In embodiments, R¹⁷ is substituted or unsubstituted aryl (e.g., C₆-C₁₀, C₁₀, or phenyl). In embodiments, R¹⁷ is substituted or unsubstituted heteroaryl (e.g., 5 to 10 membered, 5 to 9 membered, or 5 to 6 membered).

In embodiments, R¹⁷ is substituted alkyl (e.g., C₁-C₈, C₁-C₆, or C₁-C₄). In embodiments, R¹⁷ is substituted heteroalkyl (e.g., 2 to 8 membered, 2 to 6 membered, or 2 to 4 membered). In embodiments, R¹⁷ is substituted cycloalkyl (e.g., C₃-C₈, C₃-C₆, or C₅-C₆). In embodiments, R¹⁷ is substituted heterocycloalkyl (e.g., 3 to 8 membered, 3 to 6 membered, or 5 to 6 membered). In embodiments, R¹⁷ is substituted aryl (e.g., C₆-C₁₀, C,₁₀, or phenyl). In embodiments, R¹⁷ is substituted heteroaryl (e.g., 5 to 10 membered, 5 to 9 membered, or 5 to 6 membered). In embodiments, R¹⁷ is an unsubstituted alkyl (e.g., C₁-C₈, C₁-C₆, or C₁-C₄). In embodiments, R¹⁷ is an unsubstituted heteroalkyl (e.g., 2 to 8 membered, 2 to 6 membered, or 2 to 4 membered). In embodiments, R¹⁷ is an unsubstituted cycloalkyl (e.g., C₃-C₈, C₃-C₆, or C₅-C₆). In embodiments, R¹⁷ is an unsubstituted heterocycloalkyl (e.g., 3 to 8 membered, 3 to 6 membered, or 5 to 6 membered). In embodiments, R¹⁷ is an unsubstituted aryl (e.g., C₆-C₁₀, C₁₀, or phenyl). In embodiments, R¹⁷ is an unsubstituted heteroaryl (e.g., 5 to 10 membered, 5 to 9 membered, or 5 to 6 membered).

In embodiments, R^{18A} is hydrogen. In embodiments, R^{18A} is —CX₃. In embodiments, R^{18A} is —CN. In embodiments, R^{18A} is —COOH. In embodiments, R^{18A} is —CONH₂. In embodiments, R^{18A} is —CHX₂. In embodiments, R^{18A} is —CH₂X. In embodiments, R^{18A} is an unsubstituted methyl. In embodiments, R^{18A} is an unsubstituted ethyl. In embodiments, R^{18A} is an unsubstituted propyl. In embodiments, R^{18A} is an unsubstituted isopropyl. In embodiments, R^{18A} is an unsubstituted butyl. In embodiments, R^{18A} is an unsubstituted tert-butyl.

In embodiments, R^{18B} is hydrogen. In embodiments, R^{18B} is —CX₃. In embodiments, R^{18B} is —CN. In embodiments, R^{18B} is —COOH. In embodiments, R^{18B} is —CONH₂. In embodiments, R^{18A} is —CHX₂. In embodiments, R^{18B} is —CH₂X. In embodiments, R^{18B} is an unsubstituted methyl. In embodiments, R^{18B} is an unsubstituted ethyl. In embodiments, R^{18B} is an unsubstituted propyl. In embodiments, R^{18B} is an unsubstituted isopropyl. In embodiments, R^{18B} is an unsubstituted butyl. In embodiments, R^{18B} is an unsubstituted tert-butyl.

In embodiments, R^{18C} is hydrogen. In embodiments, R^{18C} is —CX₃. In embodiments, R^{18C} is —CN. In embodiments, R^{18C} is —COOH. In embodiments, R^{18C} is —CONH₂. In embodiments, R^{18C} is —CHX₂. In embodiments, R^{18C} is —CH₂X. In embodiments, R^{18C} is an unsubstituted methyl. In embodiments, R^{18C} is an unsubstituted ethyl. In embodiments, R^{18C} is an unsubstituted propyl. In embodiments, R^{18C} is an unsubstituted isopropyl. In embodiments, R^{18C} is an unsubstituted butyl. In embodiments, R^{18C} is an unsubstituted tert-butyl.

In embodiments, R^{18D} is hydrogen. In embodiments, R^{18D} is —CX₃. In embodiments, R^{18D} is —CN. In embodiments, R^{18D} is —COOH. In embodiments, R^{18D} is —CONH₂. In embodiments, R^{18D} is —CHX₂. In embodiments, R^{18D} is —CH₂X. In embodiments, R^{18D} is an unsubstituted methyl. In embodiments, R^{18D} is an unsubstituted ethyl. In embodiments, R^{18D} is an unsubstituted propyl. In embodiments, R^{18D} is an unsubstituted isopropyl. In embodiments, R^{18D} is an unsubstituted butyl. In embodiments, R^{18D} is an unsubstituted tert-butyl.

In embodiments, R¹⁸ is independently hydrogen, oxo, halogen, —CX¹⁸₃, —CHX¹⁸₂, —OCH₂X¹⁸, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁸₃, —OCHX¹⁸₂, R⁸¹-substituted or unsubstituted alkyl (e.g., C₁-C₈, C₁-C₆, or C₁-C₄), R⁸¹-substituted or unsubstituted heteroalkyl (e.g., 2 to 8 membered, 2 to 6 membered, or 2 to 4 membered), R⁸¹-substituted or unsubstituted cycloalkyl (e.g., C₃-C₈, C₃-C₆, or C₅-C₆), R⁸¹-substituted or unsubstituted heterocycloalkyl (e.g., 3 to 8 membered, 3 to 6 membered, or 5 to 6 membered), R⁸¹-substituted or unsubstituted aryl (e.g., C₆-C₁₀, C₁₀, or phenyl), or R⁸¹-substituted or unsubstituted heteroaryl (e.g., 5 to 10 membered, 5 to 9 membered, or 5 to 6 membered). X¹⁸ is halogen. In embodiments, X¹⁸ is F.

R⁸¹ is independently oxo, halogen, —CX⁸¹₃, —CHX⁸¹₂, —OCH₂X⁸¹, —OCHX⁸¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸¹₃, —OCHX⁸¹₂, R⁸²-substituted or unsubstituted alkyl (e.g., C₁-C₈, C₁-C₆, or C₁-C₄), R⁸²-substituted or unsubstituted heteroalkyl (e.g., 2 to 8 membered, 2 to 6 membered, or 2 to 4 membered), R⁸²-substituted or unsubstituted cycloalkyl (e.g., C₃-C₈, C₃-C₆, or C₅-C₆), R⁸²-substituted or unsubstituted heterocycloalkyl (e.g., 3 to 8 membered, 3 to 6 membered, or 5 to 6 membered), R⁸²-substituted or unsubstituted aryl (e.g., C₆-C₁₀, C₁₀, or phenyl), or R⁸²-substituted or unsubstituted heteroaryl (e.g., 5 to 10 membered, 5 to 9 membered, or 5 to 6 membered). X⁸¹ is halogen. In embodiments, X⁸¹ is F.

R⁸² is independently oxo, halogen, —CX⁸²₃, —CHX⁸²₂, —OCH₂X⁸², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸²₃, —OCHX⁸²₂, R⁸³-substituted or unsubstituted alkyl (e.g., C₁-C₈, C₁-C₆, or C₁-C₄), R⁸³-substituted or unsubstituted heteroalkyl (e.g., 2 to 8 membered, 2 to 6 membered, or 2 to 4 membered), R⁸³-substituted or unsubstituted cycloalkyl (e.g., C₃-C₈, C₃-C₆, or C₅-C₆), R⁸³-substituted or unsubstituted heterocycloalkyl (e.g., 3 to 8 membered, 3 to 6 membered, or 5 to 6 membered), R⁸³-substituted or unsubstituted aryl (e.g., C₆-C₁₀, C₁₀, or phenyl), or R⁸³-substituted or unsubstituted heteroaryl (e.g., 5 to 10 membered, 5 to 9 membered, or 5 to 6 membered). X⁸² is halogen. In embodiments, X⁸² is F.

In embodiments, R^{18A} is independently hydrogen, oxo, halogen, —CX^{18A}₃, —CHX^{18A}₂, —OCH₂X^{18A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18A}₃, —OCHX^{18A}₂, R^{81A}-substituted or unsubstituted alkyl (e.g., C₁-C₈, C₁-C₆, or C₁-C₄), R^{8 1A}-substituted or unsubstituted heteroalkyl (e.g., 2 to 8 membered, 2 to 6 membered, or 2 to 4 membered), R^{81A}-substituted or unsubstituted cycloalkyl (e.g., C₃-C₈, C₃-C₆, or C₅-C₆), R^{81A}-substituted or unsubstituted heterocycloalkyl (e.g., 3 to 8 membered, 3 to 6 membered, or 5 to 6 membered), R^{81A}-substituted or unsubstituted aryl (e.g., C₆-C₁₀, C₁₀, or phenyl), or R^{81A}-substituted or unsubstituted heteroaryl (e.g., 5 to 10 membered, 5 to 9 membered, or 5 to 6 membered). X^{18A} is halogen. In embodiments, X^{18A} is F.

R^{81A} is independently oxo, halogen, —CX^{81A}₃, —CHX^{81A}₂, —OCH₂X^{81A}, —OCHX^{81A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81A}₃, —OCHX^{81A}₂, R^{82A}-substituted or unsubstituted alkyl (e.g., C₁-C₈, C₁-C₆, or C₁-C₄), R^{82A}-substituted or unsubstituted heteroalkyl (e.g., 2 to 8 membered, 2 to 6 membered, or 2 to 4 membered), R^{82A}-substituted or unsubstituted cycloalkyl (e.g., C₃-C₈, C₃-C₆, or C₅-C₆), R^{82A}-substituted or unsubstituted heterocycloalkyl (e.g., 3 to 8 membered, 3 to 6 membered, or 5 to 6 membered), R^{82A}-substituted or unsubstituted aryl (e.g., C₆-C₁₀, C₁₀, or phenyl), or R^{82A}-substituted or unsubstituted heteroaryl (e.g., 5 to 10 membered, 5 to 9 membered, or 5 to 6 membered). X^{81A} is halogen. In embodiments, X^{81A} is F.

R^{82A} is independently oxo, halogen, —CX^{82A}₃, —CHX^{82A}₂, —OCH₂X^{82A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82A}₃, —OCHX^{82A}₂, R^{83A}-substituted or unsubstituted alkyl (e.g., C₁-C₈, C₁-C₆, or C₁-C₄), R^{8 3A}-substituted or unsubstituted heteroalkyl (e.g., 2 to 8 membered, 2 to 6 membered, or 2 to 4 membered), R^{83A}-substituted or unsubstituted cycloalkyl (e.g., C₃-C₈, C₃-C₆, or C₅-C₆), R^{83A}-substituted or unsubstituted heterocycloalkyl (e.g., 3 to 8 membered, 3 to 6 membered, or 5 to 6 membered), R^{83A}-substituted or unsubstituted aryl (e.g., C₆-C₁₀, C₁₀, or phenyl), or R^{83A}-substituted or unsubstituted heteroaryl (e.g., 5 to 10 membered, 5 to 9 membered, or 5 to 6 membered). X^{82A} is halogen. In embodiments, X^{82A} is F.

In embodiments, R^{18B} is independently hydrogen, oxo, halogen, —CX^{18B}₃, —CHX^{18B}₂, —OCH₂X^{18B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18B}₃, —OCHX^{18B}₂, R^{81B}-substituted or unsubstituted alkyl (e.g., C₁-C₈, C₁-C₆, or C₁-C₄), R^{81B}-substituted or unsubstituted heteroalkyl (e.g., 2 to 8 membered, 2 to 6 membered, or 2 to 4 membered), R^{81B}-substituted or unsubstituted cycloalkyl (e.g., C₃-C₈, C₃-C₆, or C₅-C₆), R^{81B}-substituted or unsubstituted heterocycloalkyl (e.g., 3 to 8 membered, 3 to 6 membered, or 5 to 6 membered), R^{81B}-substituted or unsubstituted aryl (e.g., C₆-C₁₀, C₁₀, or phenyl), or R^{81B}-substituted or unsubstituted heteroaryl (e.g., 5 to 10 membered, 5 to 9 membered, or 5 to 6 membered). X^{18B} is halogen. In embodiments, X^{18B} is F.

R^{81B} is independently oxo, halogen, —CX^{81B}₃, —CHX^{81B}₂, —OCH₂X^{81B}, —OCHX^{81B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81B}₃, —OCHX^{81B}₂, R^{82B}-substituted or unsubstituted alkyl (e.g., C₁-C₈, C₁-C₆, or C₁-C₄), R^{82B}-substituted or unsubstituted heteroalkyl (e.g., 2 to 8 membered, 2 to 6 membered, or 2 to 4 membered), R^{82B}-substituted or unsubstituted cycloalkyl (e.g., C₃-C₈, C₃-C₆, or C₅-C₆), R^{82B}-substituted or unsubstituted heterocycloalkyl (e.g., 3 to 8 membered, 3 to 6 membered, or 5 to 6 membered), R^{82B}-substituted or unsubstituted aryl (e.g., C₆-C₁₀, C₁₀, or phenyl), or R^{82B}-substituted or unsubstituted heteroaryl (e.g., 5 to 10 membered, 5 to 9 membered, or 5 to 6 membered). X^{81B} is halogen. In embodiments, X^{81B} is F.

R^{82B} is independently oxo, halogen, —CX^{82B}₃, —CHX^{82B}₂, —OCH₂X^{82B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82B}₃, —OCHX^{82B}₂, R^{83B}-substituted or unsubstituted alkyl (e.g., C₁-C₈, C₁-C₆, or C₁-C₄), R^{83B}-substituted or unsubstituted heteroalkyl (e.g., 2 to 8 membered, 2 to 6 membered, or 2 to 4 membered), R^{83B}-substituted or unsubstituted cycloalkyl (e.g., C₃-C₈, C₃-C₆, or C₅-C₆), R^{83B}-substituted or unsubstituted heterocycloalkyl (e.g., 3 to 8 membered, 3 to 6 membered, or 5 to 6 membered), R^{83B}-substituted or unsubstituted aryl (e.g., C₆-C₁₀, C₁₀, or phenyl), or R^{83B}-substituted or unsubstituted heteroaryl (e.g., 5 to 10 membered, 5 to 9 membered, or 5 to 6 membered). X^{82B} is halogen. In embodiments, X^{82B} is F.

In embodiments, R^{18C} is independently hydrogen, oxo, halogen, —CX^{18C}₃, —CHX^{18C}₂, —OCH₂X^{18C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18C}₃, —OCHX^{18C}₂, R^{81C}-substituted or unsubstituted alkyl (e.g., C₁-C₈, C₁-C₆, or C₁-C₄), R^{81C}-substituted or unsubstituted heteroalkyl (e.g., 2 to 8 membered, 2 to 6 membered, or 2 to 4 membered), R^{81C}-substituted or unsubstituted cycloalkyl (e.g., C₃-C₈, C₃-C₆, or C₅-C₆), R^{81C}-substituted or unsubstituted heterocycloalkyl (e.g., 3 to 8 membered, 3 to 6 membered, or 5 to 6 membered), R^{81C}-substituted or unsubstituted aryl (e.g., C₆-C₁₀, C₁₀, or phenyl), or R^{81C}-substituted or unsubstituted heteroaryl (e.g., 5 to 10 membered, 5 to 9 membered, or 5 to 6 membered). X^{18C} is halogen. In embodiments, X^{18C} is F.

R^{81C} is independently oxo, halogen, —CX^{81C}₃, —CHX^{81C}₂, —OCH₂X^{81C}, —OCHX^{81C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81C}₃, —OCHX^{81C}₂, R^{82C}-substituted or unsubstituted alkyl (e.g., C₁-C₈, C₁-C₆, or C₁-C₄), R^{82C}-substituted or unsubstituted heteroalkyl (e.g., 2 to 8 membered, 2 to 6 membered, or 2 to 4 membered), R^{82C}-substituted or unsubstituted cycloalkyl (e.g., C₃-C₈, C₃-C₆, or C₅-C₆), R^{82C}-substituted or unsubstituted heterocycloalkyl (e.g., 3 to 8 membered, 3 to 6 membered, or 5 to 6 membered), R^{82C}-substituted or unsubstituted aryl (e.g., C₆-C₁₀, C₁₀, or phenyl), or R^{82C}-substituted or unsubstituted heteroaryl (e.g., 5 to 10 membered, 5 to 9 membered, or 5 to 6 membered). X^{81C} is halogen. In embodiments, X^{81C} is F.

R^{82C} is independently oxo, halogen, —CX^{82C}₃, —CHX^{82C}₂, —OCH₂X^{82C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82C}₃, —OCHX^{82C}₂, R^{83C}-substituted or unsubstituted alkyl (e.g., C₁-C₈, C₁-C₆, or C₁-C₄), R^{83C}-substituted or unsubstituted heteroalkyl (e.g., 2 to 8 membered, 2 to 6 membered, or 2 to 4 membered), R^{83C}-substituted or unsubstituted cycloalkyl (e.g., C₃-C₈, C₃-C₆, or C₅-C₆), R^{83C}-substituted or unsubstituted heterocycloalkyl (e.g., 3 to 8 membered, 3 to 6 membered, or 5 to 6 membered), R^{83C}-substituted or unsubstituted aryl (e.g., C₆-C₁₀, C₁₀, or phenyl), or R^{83C}-substituted or unsubstituted heteroaryl (e.g., 5 to 10 membered, 5 to 9 membered, or 5 to 6 membered). X^{82C} is halogen. In embodiments, X^{82C} is F.

In embodiments, R^{18D} is independently hydrogen, oxo, halogen, —CX^{18D}₃, —CHX^{18D}₂, —OCH₂X^{18D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18D}₃, —OCHX^{18D}₂, R^{81D}-substituted or unsubstituted alkyl (e.g., C₁-C₈, C₁-C₆, or C₁-C₄), R^{81D}-substituted or unsubstituted heteroalkyl (e.g., 2 to 8 membered, 2 to 6 membered, or 2 to 4 membered), R^{81D}-substituted or unsubstituted cycloalkyl (e.g., C₃-C₈, C₃-C₆, or C₅-C₆), R^{81D}-substituted or unsubstituted heterocycloalkyl (e.g., 3 to 8 membered, 3 to 6 membered, or 5 to 6 membered), R^{81D}-substituted or unsubstituted aryl (e.g., C₆-C₁₀, C₁₀, or phenyl), or R^{81D}-substituted or unsubstituted heteroaryl (e.g., 5 to 10 membered, 5 to 9 membered, or 5 to 6 membered). X^{18D} is halogen. In embodiments, X^{18D} is F.

R^{81D} is independently oxo, halogen, —CX^{81D}₃, —CHX^{81D}₂, —OCH₂X^{81D}, —OCHX^{81D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81D}₃, —OCHX^{81D}₂, R^{82D}-substituted or unsubstituted alkyl (e.g., C₁-C₈, C₁-C₆, or C₁-C₄), R^{82D}-substituted or unsubstituted heteroalkyl (e.g., 2 to 8 membered, 2 to 6 membered, or 2 to 4 membered), R^{82D}-substituted or unsubstituted cycloalkyl (e.g., C₃-C₈, C₃-C₆, or C₅-C₆), R^{82D}-substituted or unsubstituted heterocycloalkyl (e.g., 3 to 8 membered, 3 to 6 membered, or 5 to 6 membered), R^{82D}-substituted or unsubstituted aryl (e.g., C₆-C₁₀, C₁₀, or phenyl), or R^{82D}-substituted or unsubstituted heteroaryl (e.g., 5 to 10 membered, 5 to 9 membered, or 5 to 6 membered). X^{81D} is halogen. In embodiments, X^{81D} is F.

R^{82D} is independently oxo, halogen, —CX^{82D}₃, —CHX^{82D}₂, —OCH₂X^{82D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82D}₃, —OCHX^{82D}₂, R^{83D}-substituted or unsubstituted alkyl (e.g., C₁-C₈, C₁-C₆, or C₁-C₄), R^{83D}-substituted or unsubstituted heteroalkyl (e.g., 2 to 8 membered, 2 to 6 membered, or 2 to 4 membered), R^{83D}-substituted or unsubstituted cycloalkyl (e.g., C₃-C₈, C₃-C₆, or C₅-C₆), R^{83D}-substituted or unsubstituted heterocycloalkyl (e.g., 3 to 8 membered, 3 to 6 membered, or 5 to 6 membered), R^{83D}-substituted or unsubstituted aryl (e.g., C₆-C₁₀, C₁₀, or phenyl), or R^{83D}-substituted or unsubstituted heteroaryl (e.g., 5 to 10 membered, 5 to 9 membered, or 5 to 6 membered). X^{82D} is halogen. In embodiments, X^{82D} is F.

R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, and R^{83D} are independently hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In embodiments, R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, and R^{83D} are independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In embodiments, R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, and R^{83D} are independently oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, unsubstituted C₁-C₈ alkyl, unsubstituted 2 to 8 membered heteroalkyl, unsubstituted C₃-C₈ cycloalkyl, unsubstituted 3 to 6 membered heterocycloalkyl, unsubstituted phenyl, or unsubstituted 5 to 6 membered heteroaryl.

In embodiments, E is:

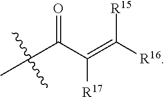

In embodiments, R¹⁵, R¹⁶, R¹⁷, and R¹⁸ are hydrogen. In embodiments, R¹⁵ is hydrogen; R¹⁶ is hydrogen, —CH₃, or —CH₂NR^{16A}R^{16B}, R¹⁷ is hydrogen; and R^{16A} and R^{16B} are independently hydrogen or unsubstituted alkyl. In embodiments, R^{16A} and R^{16B} are independently unsubstituted methyl. In embodiments, R¹⁵ is hydrogen; R¹⁶ is hydrogen; R¹⁷ is hydrogen, —CH₃, or —CH₂NR^{17A}R^{17B}, and R^{17A} and R^{17B} are independently hydrogen or unsubstituted alkyl. In embodiments, R^{17A} and R^{17B} are independently unsubstituted methyl. In embodiments, R¹⁵ is hydrogen, —CH₃, or —CH₂NR^{15A}R^{15B}, R¹⁶ is hydrogen; R¹⁷ is hydrogen; and R^{15A} and R^{15B} are independently hydrogen or unsubstituted alkyl. In embodiments, R^{15A} and R^{15B} are independently unsubstituted methyl.

In embodiments, -L¹-L²-E is:

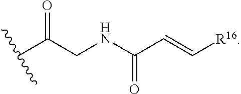

In embodiments, -L¹-L²-E is:

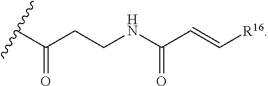

In embodiments, -L¹-L²-E is:

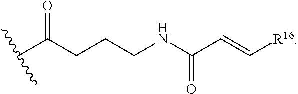

In embodiments, -E is:

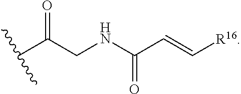

In embodiments, -E is:

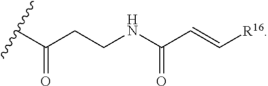

In embodiments, -E is:

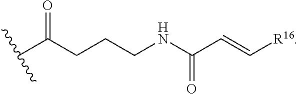

In embodiments R¹⁶ is —CH₂N(CH₃)₂. In embodiments R¹⁶ is —CH₂CH₂N(CH₃)₂.

In embodiments, X is —F. In embodiments, X is —Cl. In embodiments, X is —Br. In embodiments, X is —I. In embodiments, X¹ is —F. In embodiments, X¹ is —Cl. In embodiments, X¹ is —Br. In embodiments, X¹ is —I. In embodiments, X² is —F. In embodiments, X² is —Cl. In embodiments, X² is —Br. In embodiments, X² is —I. In embodiments, X³ is —F. In embodiments, X³ is —Cl. In embodiments, X³ is —Br. In embodiments, X³ is —I. In embodiments, X⁴ is —F. In embodiments, X⁴ is —Cl. In embodiments, X⁴ is —Br. In embodiments, X⁴ is —I. In embodiments, X⁶ is —F. In embodiments, X⁶ is —Cl. In embodiments, X⁶ is —Br. In embodiments, X⁶ is —I. In embodiments, X⁷ is —F. In embodiments, X⁷ is —Cl. In embodiments, X⁷ is —Br. In embodiments, X⁷ is —I. In embodiments, X⁸ is —F. In embodiments, X⁸ is —Cl. In embodiments, X⁸ is —Br. In embodiments, X⁸ is —I. In embodiments, X⁹ is —F. In embodiments, X⁹ is —Cl. In embodiments, X⁹ is —Br. In embodiments, X⁹ is —I. X^{4.1} is —F, —Cl, —Br, or —I. X^{4.2} is —F, —Cl, —Br, or —I. X^{4.3} is —F, —Cl, —Br, or —I. X^{4.4} is —F, —Cl, —Br, or —I. X^{4.5} is —F, —Cl, —Br, or —I. In embodiments, X^{4.1} is —F. In embodiments, X^{4.1} is —Cl. In embodiments, X^{4.1} is —Br. In embodiments, X^{4.1} is —I. In embodiments, X^{4.2} is —F. In embodiments, X^{4.2} is —Cl. In embodiments, X^{4.2} is —Br. In embodiments, X^{4.2} is —I. In embodiments, X^{4.3} is —F. In embodiments, X^{4.3} is —Cl. In embodiments, X^{4.3} is —Br. In embodiments, X^{4.3} is —I. In embodiments, X^{4.4} is —F. In embodiments, X^{4.4} is —Cl. In embodiments, X^{4.4} is —Br. In embodiments, X^{4.4} is —I. In embodiments, X^{4.5} is —F. In embodiments, X^{4.5} is —Cl. In embodiments, X^{4.5} is —Br. In embodiments, X^{4.5} is —I.

In embodiments, n1 is 0. In embodiments, n1 is 1. In embodiments, n1 is 2. In embodiments, n1 is 3. In embodiments, n1 is 4. In embodiments, n2 is 0. In embodiments, n2 is 1. In embodiments, n2 is 2. In embodiments, n2 is 3. In embodiments, n2 is 4. In embodiments, n3 is 0. In embodiments, n3 is 1. In embodiments, n3 is 2. In embodiments, n3 is 3. In embodiments, n3 is 4. In embodiments, n4 is 0. In embodiments, n4 is 1. In embodiments, n4 is 2. In embodiments, n4 is 3. In embodiments, n4 is 4. In embodiments, n6 is 0. In embodiments, n6 is 1. In embodiments, n6 is 2. In embodiments, n6 is 3. In embodiments, n6 is 4. In embodiments, n7 is 0. In embodiments, n7 is 1. In embodiments, n7 is 2. In embodiments, n7 is 3. In embodiments, n7 is 4. In embodiments, n8 is 0. In embodiments, n8 is 1. In embodiments, n8 is 2. In embodiments, n8 is 3. In embodiments, n8 is 4. In embodiments, n9 is 0. In embodiments, n9 is 1. In embodiments, n9 is 2. In embodiments, n9 is 3. In embodiments, n9 is 4.

In embodiments, m1 is 1. In embodiments, m1 is 2. In embodiments, m2 is 1. In embodiments, m2 is 2. In embodiments, m3 is 1. In embodiments, m3 is 2. In embodiments, m4 is 1. In embodiments, m4 is 2. In embodiments, m6 is 1. In embodiments, m6 is 2. In embodiments, m7 is 1. In embodiments, m7 is 2. In embodiments, m8 is 1. In embodiments, m8 is 2. In embodiments, m9 is 1. In embodiments, m9 is 2.

In embodiments, v3 is 1. In embodiments, v3 is 2. In embodiments, v4 is 1. In embodiments, v4 is 2. In embodiments, v6 is 1. In embodiments, v6 is 2. In embodiments, v7 is 1. In embodiments, v7 is 2. In embodiments, v8 is 1. In embodiments, v8 is 2. In embodiments, v9 is 1. In embodiments, v9 is 2.

In embodiments, z3 is 0. In embodiments, z3 is 1. In embodiments, z3 is 2. In embodiments, z3 is 3. In embodiments, z3 is 4. In embodiments z4 is an integer from 0 to 2. In embodiments, z4 is 0 or 1. In embodiments, z4 is 0. In embodiments, z4 is 1. In embodiments, z4 is 2. In embodiments, z4 is 3. In embodiments, z4 is 4. In embodiments, z4 is 5. In embodiments, z20 is 0. In embodiments, z20 is 1. In embodiments, z20 is 2. In embodiments, z20 is 3. In embodiments, z20 is 4. In embodiments, z20 is 5.

In embodiments, the compound name may be written with a dash (e.g., 8-091) or without a dash (e.g., 8091) and it is understood both of these labels refer to the same compound.

In embodiments, the compound has the formula:

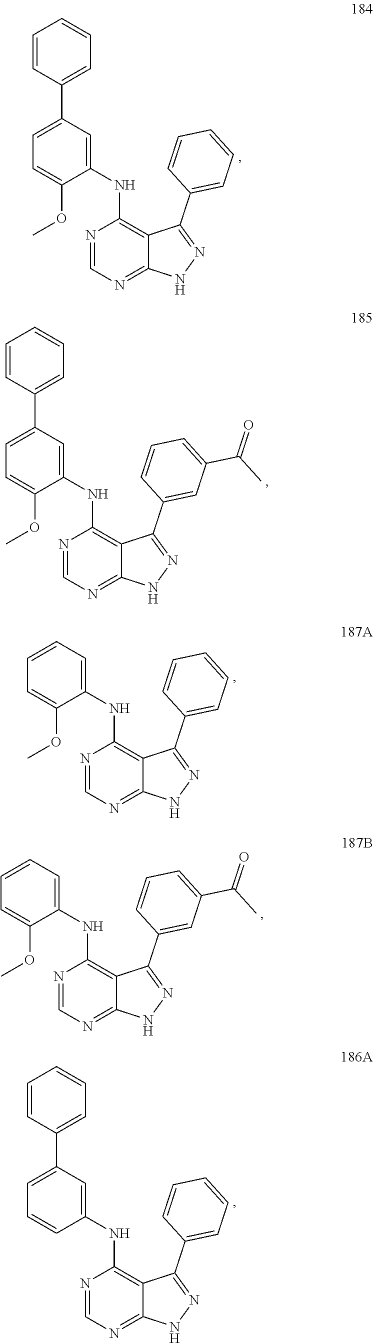 [SMILES: COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12 ; COc1ccccc1Nc1ncnc2[nH]nc(-c3ccccc3)c12 ; COc1ccccc1Nc1ncnc2[nH]nc(-c3cccc(C(C)=O)c3)c12 ; c1ccc(-c2cccc(Nc3ncnc4[nH]nc(-c5ccccc5)c34)c2)cc1]

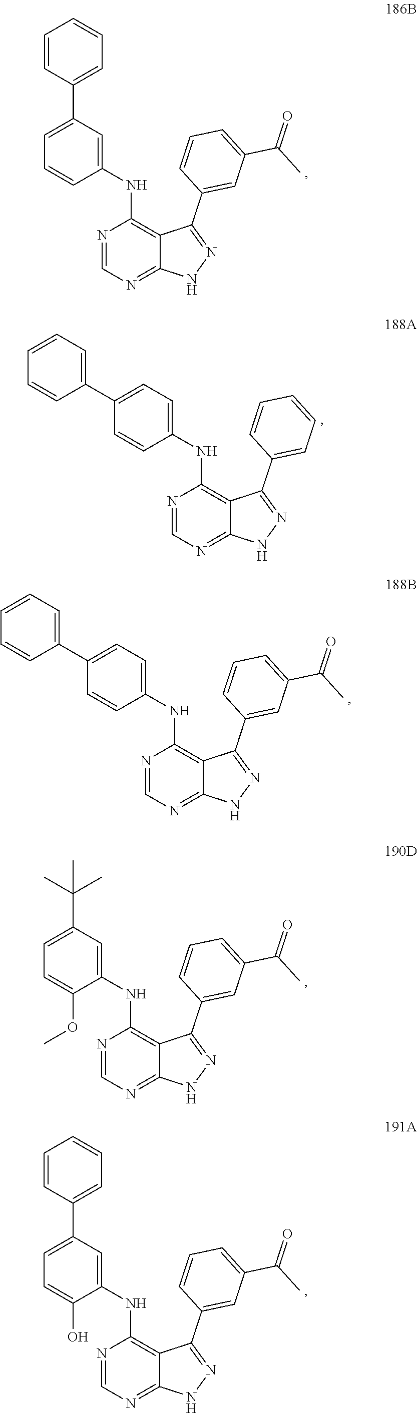 [SMILES: CC(=O)c1cccc(-c2n[nH]c3ncnc(Nc4cccc(-c5ccccc5)c4)c23)c1 ; c1ccc(-c2ccc(Nc3ncnc4[nH]nc(-c5ccccc5)c34)cc2)cc1 ; CC(=O)c1cccc(-c2n[nH]c3ncnc(Nc4ccc(-c5ccccc5)cc4)c23)c1 ; COc1ccc(C(C)(C)C)cc1Nc1ncnc2[nH]nc(-c3cccc(C(C)=O)c3)c12 ; CC(=O)c1cccc(-c2n[nH]c3ncnc(Nc4cc(-c5ccccc5)ccc4O)c23)c1]

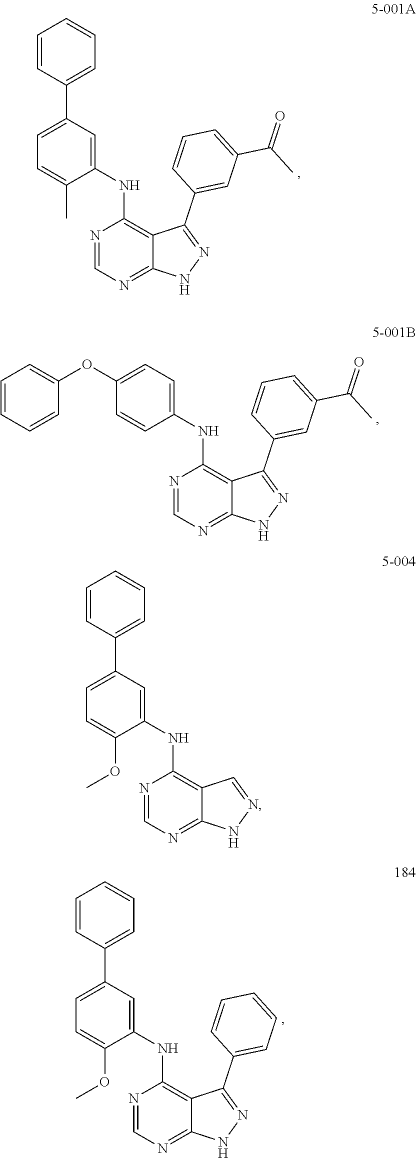 [SMILES: CC(=O)c1cccc(-c2n[nH]c3ncnc(Nc4cc(-c5ccccc5)ccc4C)c23)c1 ; CC(=O)c1cccc(-c2n[nH]c3ncnc(Nc4ccc(Oc5ccccc5)cc4)c23)c1 ; COc1ccc(-c2ccccc2)cc1Nc1ncnc2[nH]ncc12 ; COc1ccc(-c2ccccc2)cc1Nc1ncnc2[nH]nc(-c3ccccc3)c12]

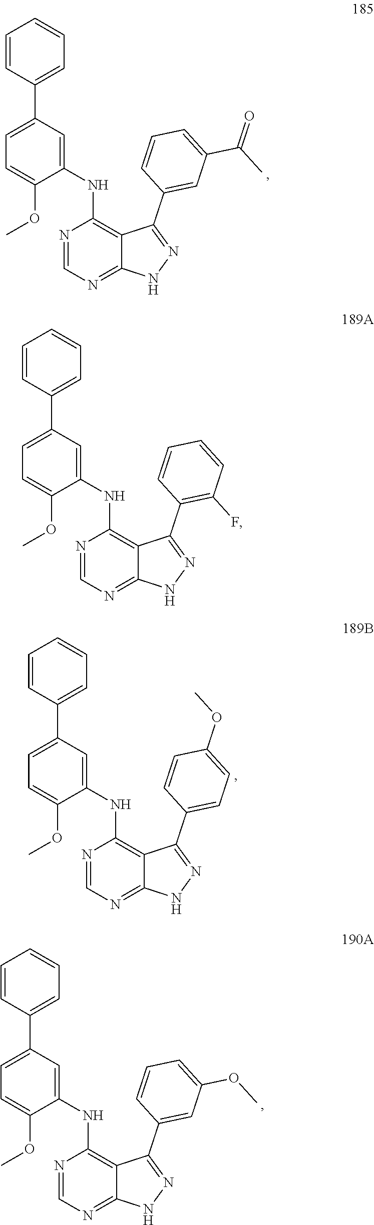 [SMILES: COc1ccc(-c2ccccc2)cc1Nc1ncnc2[nH]nc(-c3cccc(C(C)=O)c3)c12 ; COc1ccc(-c2ccccc2)cc1Nc1ncnc2[nH]nc(-c3ccccc3F)c12 ; COc1ccc(-c2n[nH]c3ncnc(Nc4cc(-c5ccccc5)ccc4OC)c23)cc1 ; COc1cccc(-c2n[nH]c3ncnc(Nc4cc(-c5ccccc5)ccc4OC)c23)c1]

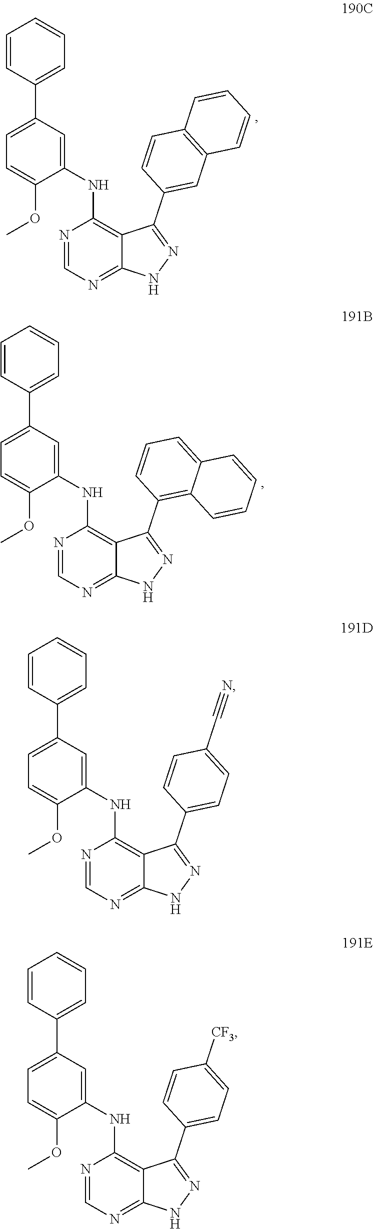 [SMILES: COc1ccc(-c2ccccc2)cc1Nc1ncnc2[nH]nc(-c3ccc4ccccc4c3)c12 ; COc1ccc(-c2ccccc2)cc1Nc1ncnc2[nH]nc(-c3cccc4ccccc34)c12 ; COc1ccc(-c2ccccc2)cc1Nc1ncnc2[nH]nc(-c3ccc(C#N)cc3)c12 ; COc1ccc(-c2ccccc2)cc1Nc1ncnc2[nH]nc(-c3ccc(C(F)(F)F)cc3)c12]

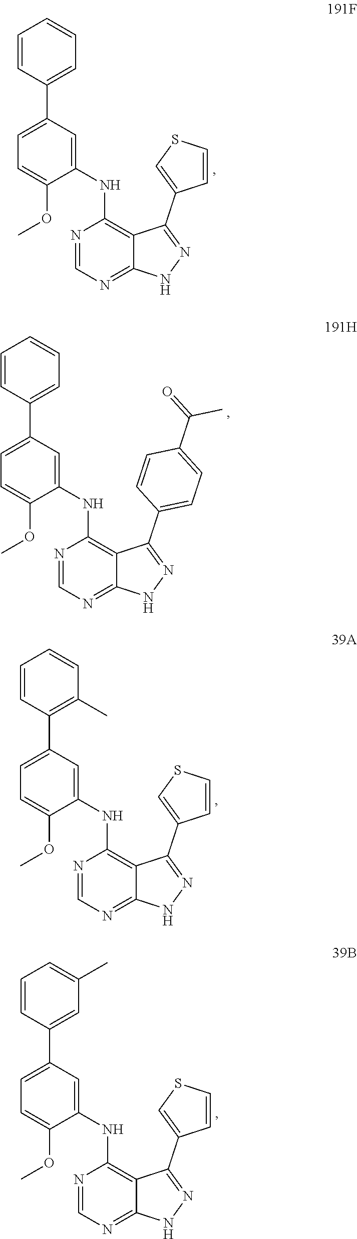

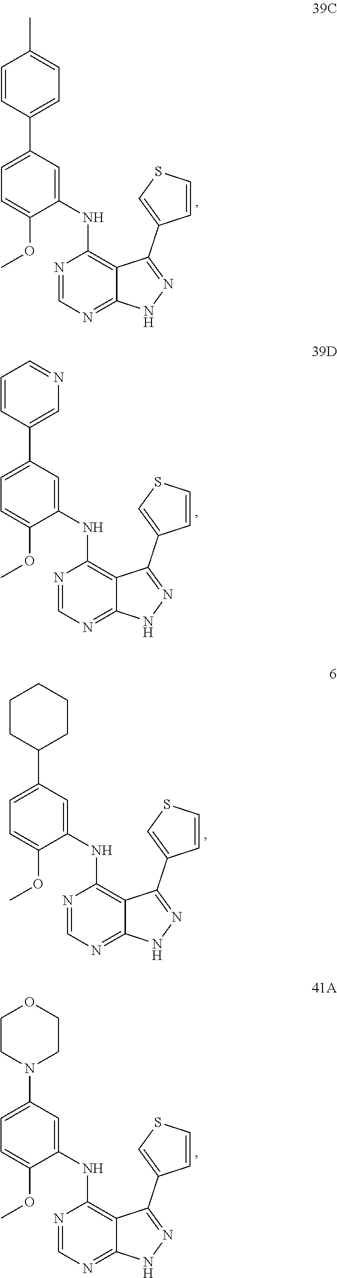 [SMILES: COc1ccc(-c2ccc(C)cc2)cc1Nc1ncnc2[nH]nc(-c3ccsc3)c12 ; COc1ccc(-c2cccnc2)cc1Nc1ncnc2[nH]nc(-c3ccsc3)c12 ; COc1ccc(C2CCCCC2)cc1Nc1ncnc2[nH]nc(-c3ccsc3)c12 ; COc1ccc(N2CCOCC2)cc1Nc1ncnc2[nH]nc(-c3ccsc3)c12]

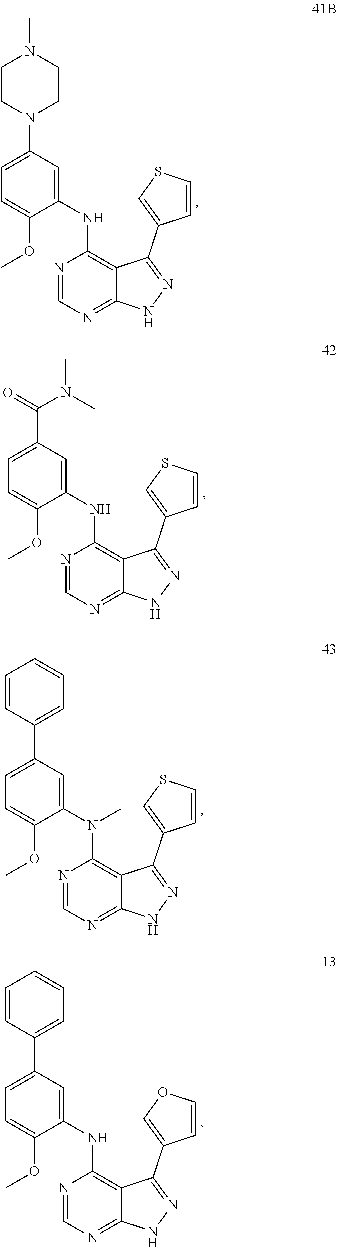 [SMILES: COc1ccc(N2CCN(C)CC2)cc1Nc1ncnc2[nH]nc(-c3ccsc3)c12 ; COc1ccc(C(=O)N(C)C)cc1Nc1ncnc2[nH]nc(-c3ccsc3)c12 ; COc1ccc(-c2ccccc2)cc1N(C)c1ncnc2[nH]nc(-c3ccsc3)c12 ; COc1ccc(-c2ccccc2)cc1Nc1ncnc2[nH]nc(-c3ccoc3)c12]

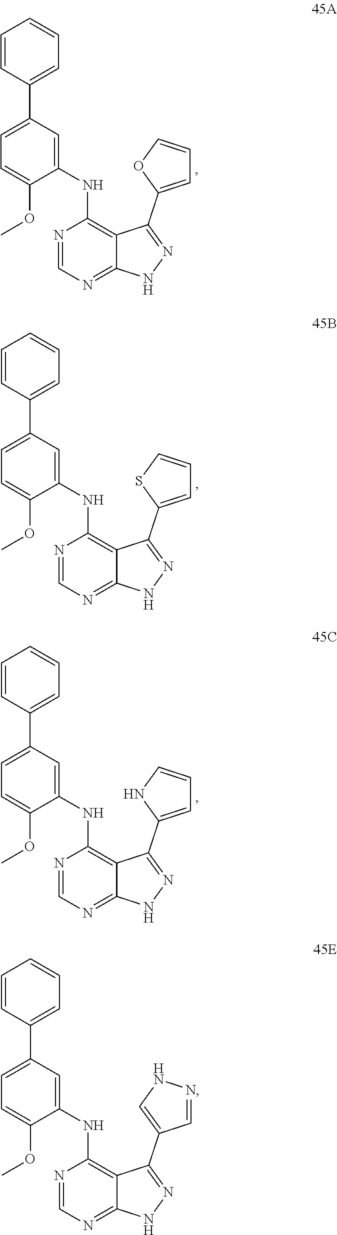 [SMILES: COc1ccc(-c2ccccc2)cc1Nc1ncnc2[nH]nc(-c3ccco3)c12 ; COc1ccc(-c2ccccc2)cc1Nc1ncnc2[nH]nc(-c3cccs3)c12 ; COc1ccc(-c2ccccc2)cc1Nc1ncnc2[nH]nc(-c3ccc[nH]3)c12 ; COc1ccc(-c2ccccc2)cc1Nc1ncnc2[nH]nc(-c3cn[nH]c3)c12]

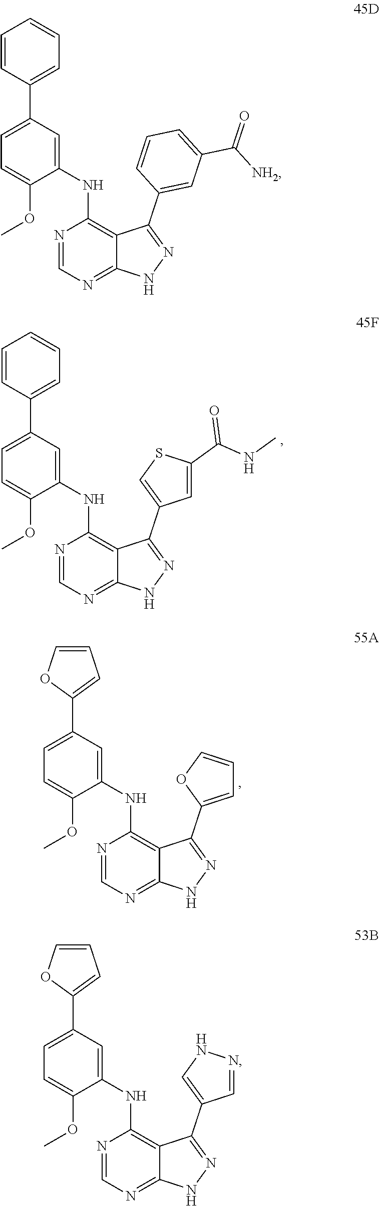 [SMILES: COc1ccc(-c2ccccc2)cc1Nc1ncnc2[nH]nc(-c3cccc(C(N)=O)c3)c12 ; CNC(=O)c1cc(-c2n[nH]c3ncnc(Nc4cc(-c5ccccc5)ccc4OC)c23)cs1 ; COc1ccc(-c2ccco2)cc1Nc1ncnc2[nH]nc(-c3ccco3)c12 ; COc1ccc(-c2ccco2)cc1Nc1ncnc2[nH]nc(-c3cn[nH]c3)c12]

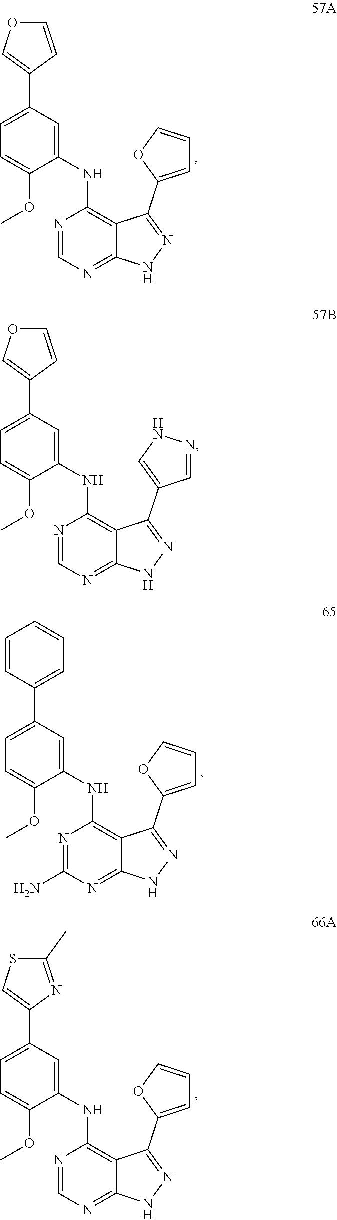 [SMILES: COc1ccc(-c2ccoc2)cc1Nc1ncnc2[nH]nc(-c3ccco3)c12 ; COc1ccc(-c2ccoc2)cc1Nc1ncnc2[nH]nc(-c3cn[nH]c3)c12 ; COc1ccc(-c2ccccc2)cc1Nc1nc(N)nc2[nH]nc(-c3ccco3)c12 ; COc1ccc(-c2csc(C)n2)cc1Nc1ncnc2[nH]nc(-c3ccco3)c12]

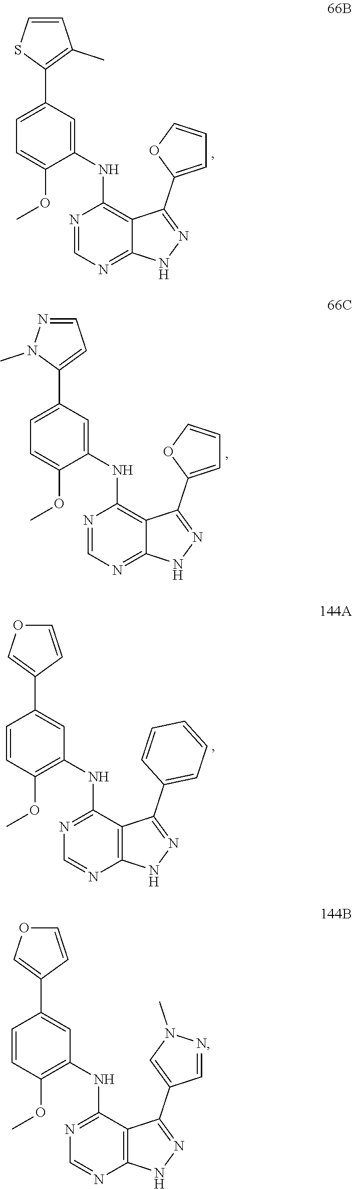 [SMILES: COc1ccc(-c2sccc2C)cc1Nc1ncnc2[nH]nc(-c3ccco3)c12 ; COc1ccc(-c2ccnn2C)cc1Nc1ncnc2[nH]nc(-c3ccco3)c12 ; COc1ccc(-c2ccoc2)cc1Nc1ncnc2[nH]nc(-c3ccccc3)c12 ; COc1ccc(-c2ccoc2)cc1Nc1ncnc2[nH]nc(-c3cnn(C)c3)c12]

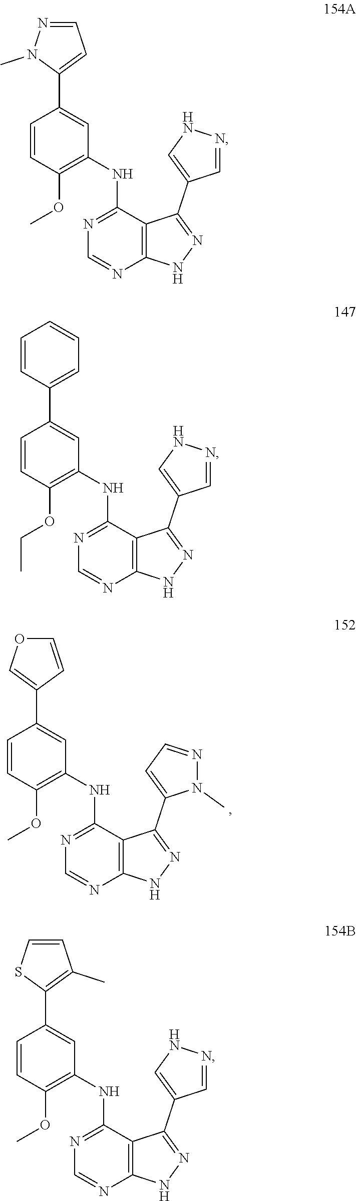 [SMILES: COc1ccc(-c2ccnn2C)cc1Nc1ncnc2[nH]nc(-c3cn[nH]c3)c12 ; CCOc1ccc(-c2ccccc2)cc1Nc1ncnc2[nH]nc(-c3cn[nH]c3)c12 ; COc1ccc(-c2ccoc2)cc1Nc1ncnc2[nH]nc(-c3ccnn3C)c12 ; COc1ccc(-c2sccc2C)cc1Nc1ncnc2[nH]nc(-c3cn[nH]c3)c12]

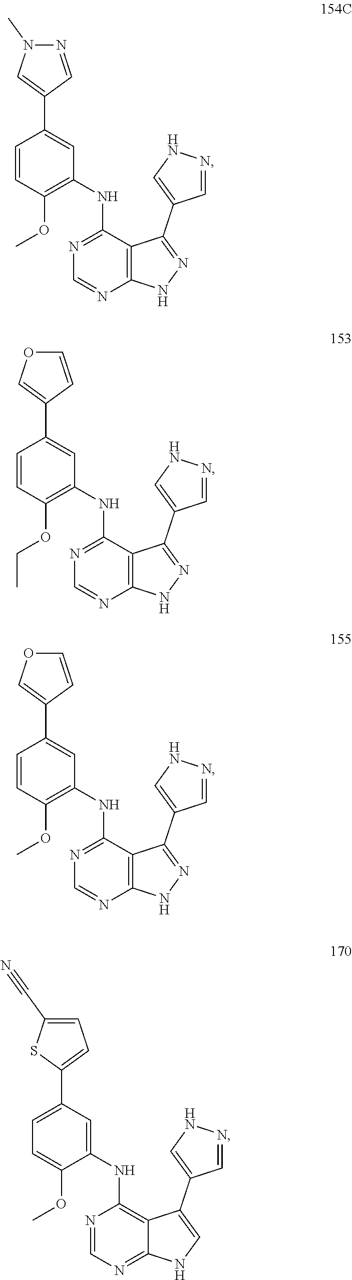 [SMILES: COc1ccc(-c2cnn(C)c2)cc1Nc1ncnc2[nH]nc(-c3cn[nH]c3)c12 ; CCOc1ccc(-c2ccoc2)cc1Nc1ncnc2[nH]nc(-c3cn[nH]c3)c12 ; COc1ccc(-c2ccoc2)cc1Nc1ncnc2[nH]nc(-c3cn[nH]c3)c12 ; COc1ccc(-c2ccc(C#N)s2)cc1Nc1ncnc2[nH]cc(-c3cn[nH]c3)c12]

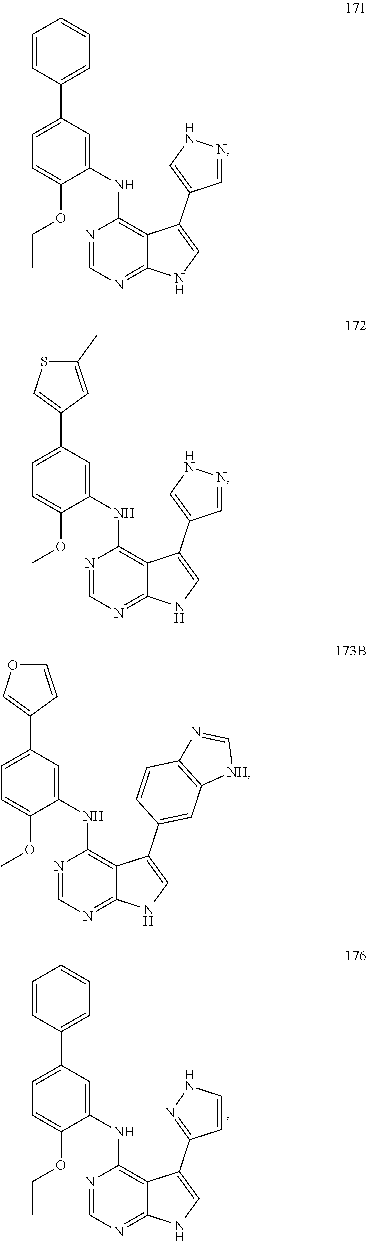 [SMILES: CCOc1ccc(-c2ccccc2)cc1Nc1ncnc2[nH]cc(-c3cn[nH]c3)c12 ; COc1ccc(-c2csc(C)c2)cc1Nc1ncnc2[nH]cc(-c3cn[nH]c3)c12 ; COc1ccc(-c2ccoc2)cc1Nc1ncnc2[nH]cc(-c3ccc4nc[nH]c4c3)c12 ; CCOc1ccc(-c2ccccc2)cc1Nc1ncnc2[nH]cc(-c3cc[nH]n3)c12]

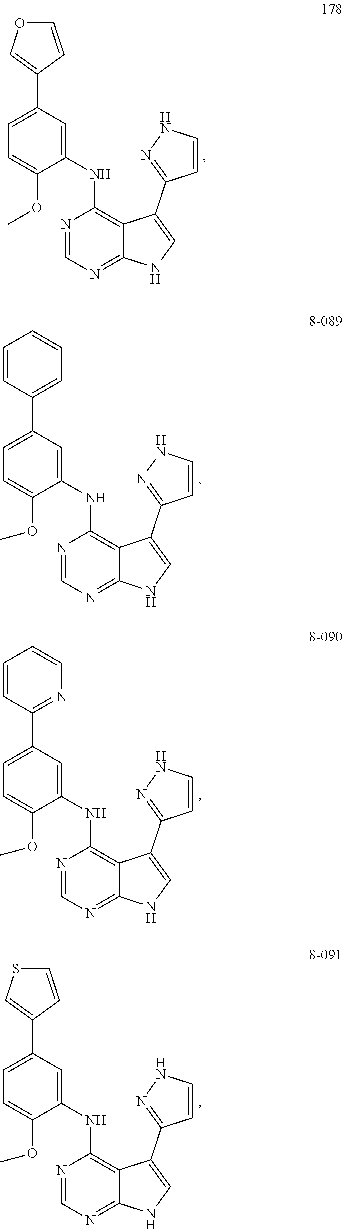 [SMILES: COc1ccc(-c2ccoc2)cc1Nc1ncnc2[nH]cc(-c3cc[nH]n3)c12 ; COc1ccc(-c2ccccc2)cc1Nc1ncnc2[nH]cc(-c3cc[nH]n3)c12 ; COc1ccc(-c2ccccn2)cc1Nc1ncnc2[nH]cc(-c3cc[nH]n3)c12 ; COc1ccc(-c2ccsc2)cc1Nc1ncnc2[nH]cc(-c3cc[nH]n3)c12]

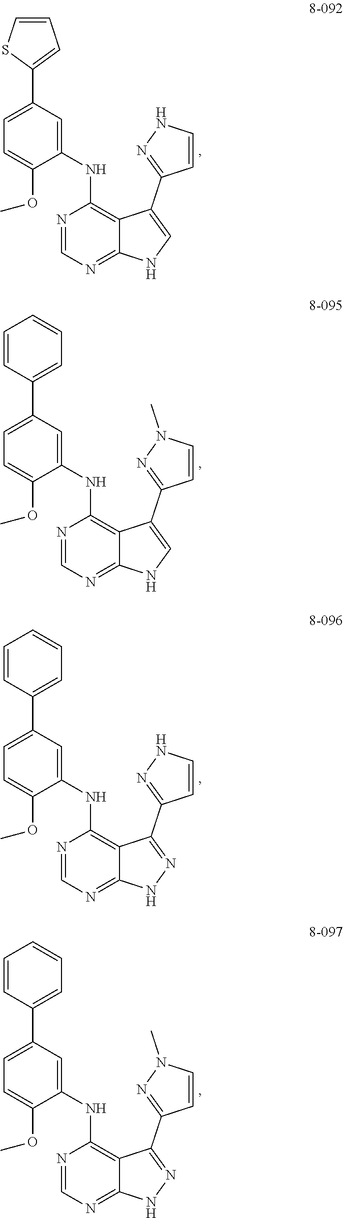 [SMILES: COc1ccc(-c2cccs2)cc1Nc1ncnc2[nH]cc(-c3cc[nH]n3)c12 ; COc1ccc(-c2ccccc2)cc1Nc1ncnc2[nH]cc(-c3ccn(C)n3)c12 ; COc1ccc(-c2ccccc2)cc1Nc1ncnc2[nH]nc(-c3cc[nH]n3)c12 ; COc1ccc(-c2ccccc2)cc1Nc1ncnc2[nH]nc(-c3ccn(C)n3)c12]

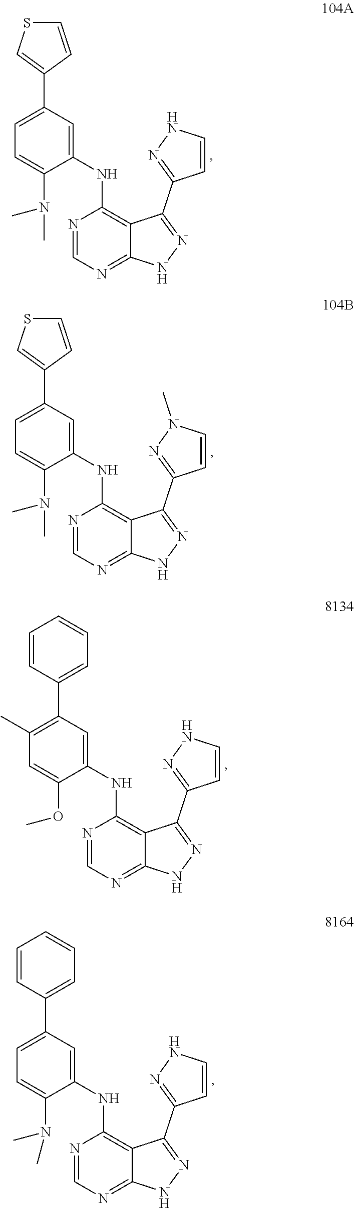 [SMILES: CN(C)c1ccc(-c2ccsc2)cc1Nc1ncnc2[nH]nc(-c3cc[nH]n3)c12 ; CN(C)c1ccc(-c2ccsc2)cc1Nc1ncnc2[nH]nc(-c3ccn(C)n3)c12 ; COc1cc(C)c(-c2ccccc2)cc1Nc1ncnc2[nH]nc(-c3cc[nH]n3)c12 ; CN(C)c1ccc(-c2ccccc2)cc1Nc1ncnc2[nH]nc(-c3cc[nH]n3)c12]

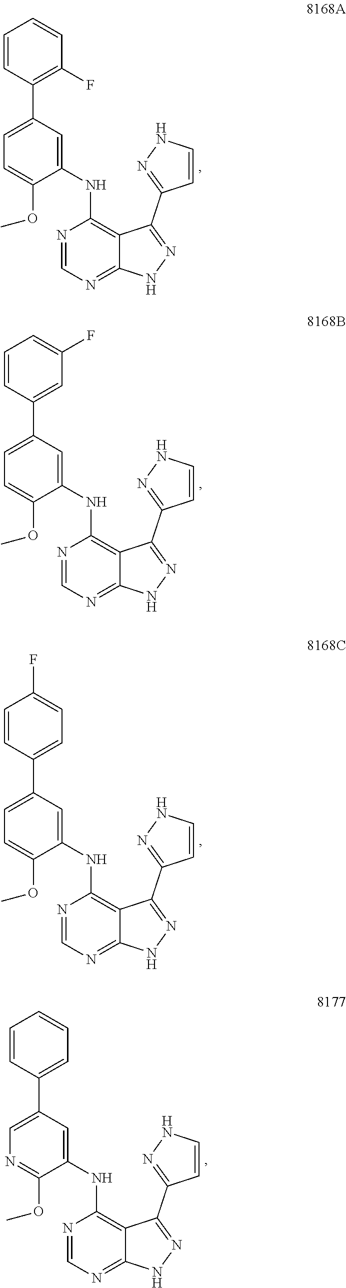 [SMILES: COc1ccc(-c2ccccc2F)cc1Nc1ncnc2[nH]nc(-c3cc[nH]n3)c12 ; COc1ccc(-c2cccc(F)c2)cc1Nc1ncnc2[nH]nc(-c3cc[nH]n3)c12 ; COc1ccc(-c2ccc(F)cc2)cc1Nc1ncnc2[nH]nc(-c3cc[nH]n3)c12 ; COc1ncc(-c2ccccc2)cc1Nc1ncnc2[nH]nc(-c3cc[nH]n3)c12]

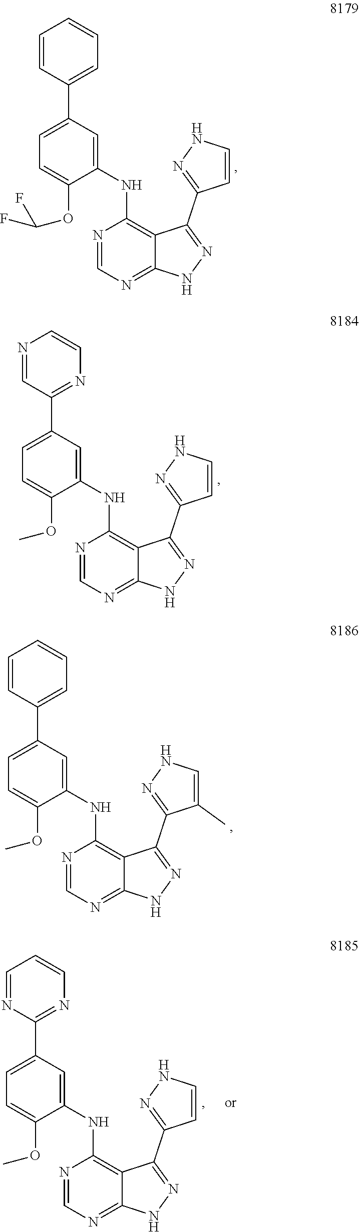

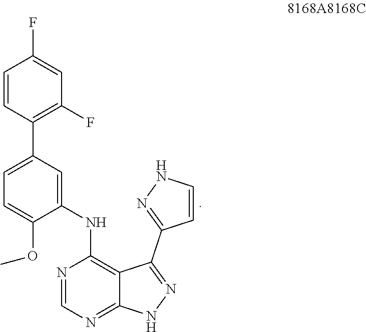 [SMILES: COc1ccc(-c2ccc(F)cc2F)cc1Nc1ncnc2[nH]nc(-c3cc[nH]n3)c12]

In embodiments, the compound has the formula:

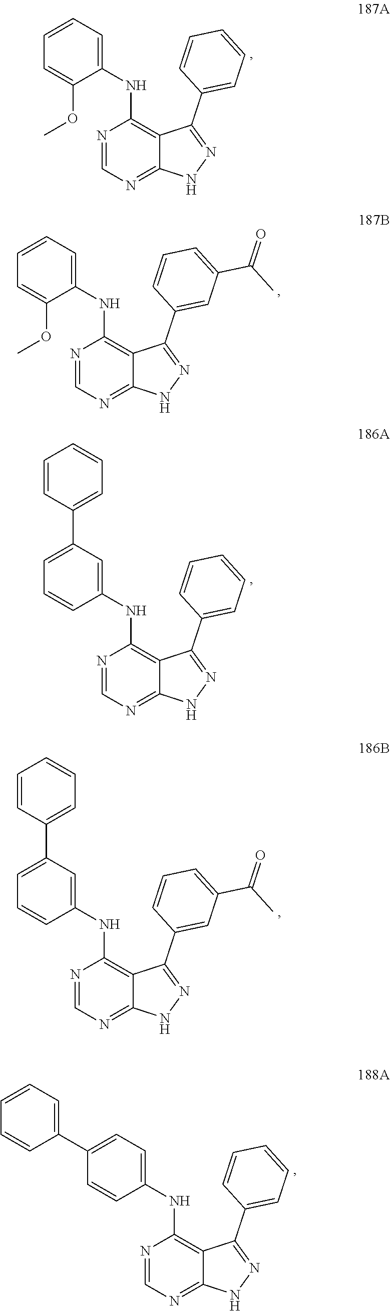 [SMILES: COc1ccccc1Nc1ncnc2[nH]nc(-c3ccccc3)c12 ; COc1ccccc1Nc1ncnc2[nH]nc(-c3cccc(C(C)=O)c3)c12 ; c1ccc(-c2cccc(Nc3ncnc4[nH]nc(-c5ccccc5)c34)c2)cc1 ; CC(=O)c1cccc(-c2n[nH]c3ncnc(Nc4cccc(-c5ccccc5)c4)c23)c1 ; c1ccc(-c2ccc(Nc3ncnc4[nH]nc(-c5ccccc5)c34)cc2)cc1]

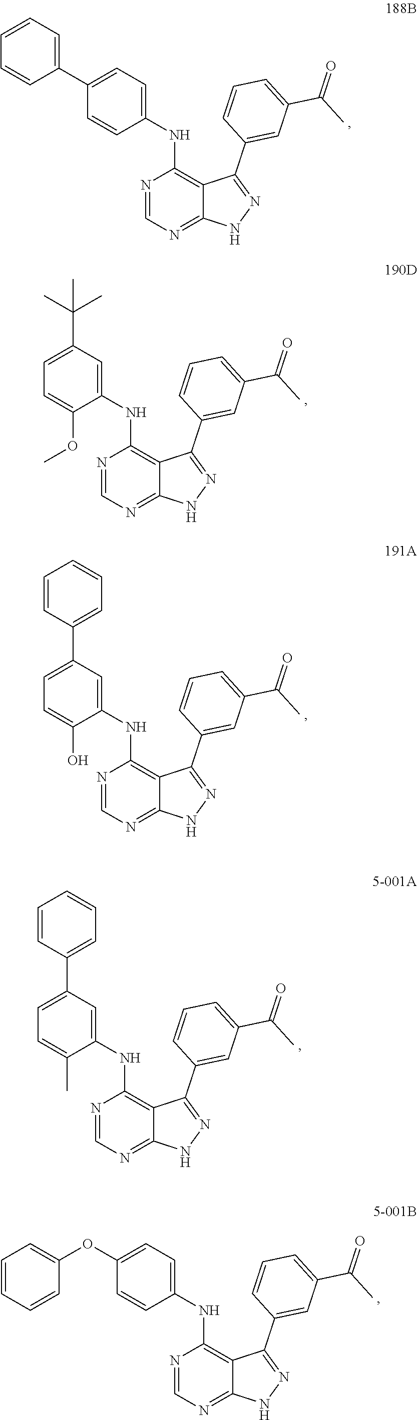 [SMILES: CC(=O)c1cccc(-c2n[nH]c3ncnc(Nc4ccc(-c5ccccc5)cc4)c23)c1 ; COc1ccc(C(C)(C)C)cc1Nc1ncnc2[nH]nc(-c3cccc(C(C)=O)c3)c12 ; CC(=O)c1cccc(-c2n[nH]c3ncnc(Nc4cc(-c5ccccc5)ccc4O)c23)c1 ; CC(=O)c1cccc(-c2n[nH]c3ncnc(Nc4cc(-c5ccccc5)ccc4C)c23)c1 ; CC(=O)c1cccc(-c2n[nH]c3ncnc(Nc4ccc(Oc5ccccc5)cc4)c23)c1]

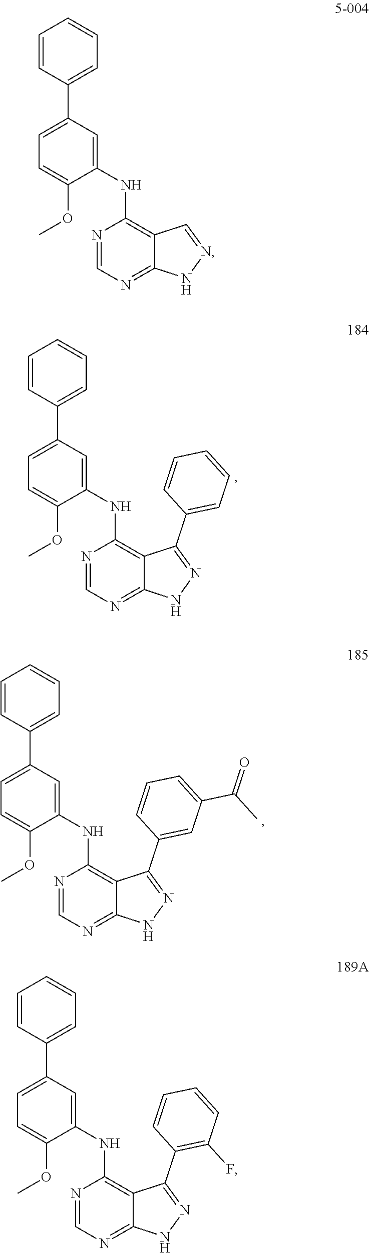 [SMILES: COc1ccc(-c2ccccc2)cc1Nc1ncnc2[nH]ncc12 ; COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12 ; COc1ccc(-c2ccccc2)cc1Nc1ncnc2[nH]nc(-c3ccccc3F)c12]

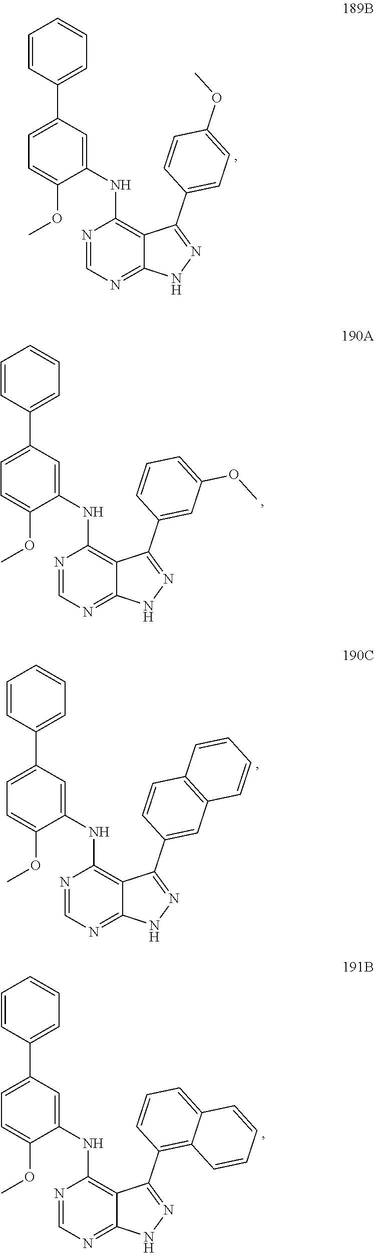 [SMILES: COc1ccc(-c2n[nH]c3ncnc(Nc4cc(-c5ccccc5)ccc4OC)c23)cc1 ; COc1cccc(-c2n[nH]c3ncnc(Nc4cc(-c5ccccc5)ccc4OC)c23)c1 ; COc1ccc(-c2ccccc2)cc1Nc1ncnc2[nH]nc(-c3ccc4ccccc4c3)c12 ; COc1ccc(-c2ccccc2)cc1Nc1ncnc2[nH]nc(-c3cccc4ccccc34)c12]

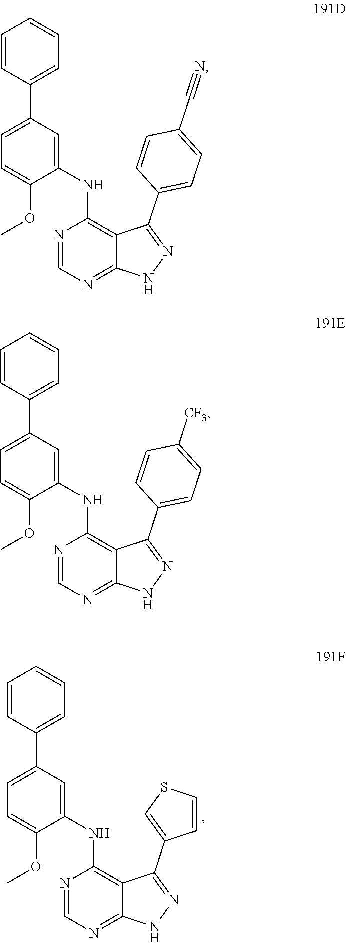 [SMILES: COc1ccc(-c2ccccc2)cc1Nc1ncnc2[nH]nc(-c3ccc(C#N)cc3)c12 ; COc1ccc(-c2ccccc2)cc1Nc1ncnc2[nH]nc(-c3ccc(C(F)(F)F)cc3)c12 ; COc1ccc(-c2ccccc2)cc1Nc1ncnc2[nH]nc(-c3ccsc3)c12]

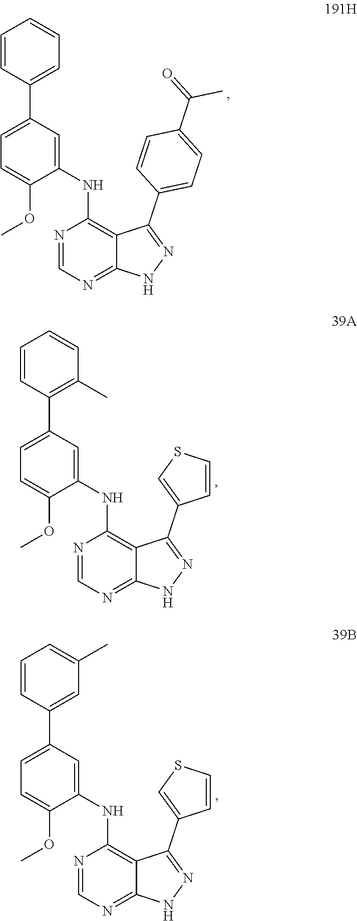

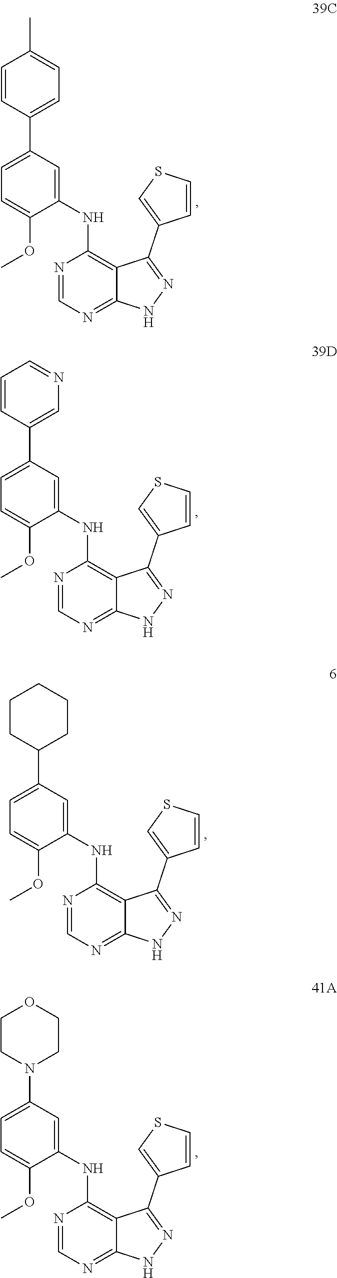 [SMILES: COc1ccc(-c2ccc(C)cc2)cc1Nc1ncnc2[nH]nc(-c3ccsc3)c12 ; COc1ccc(-c2cccnc2)cc1Nc1ncnc2[nH]nc(-c3ccsc3)c12 ; COc1ccc(C2CCCCC2)cc1Nc1ncnc2[nH]nc(-c3ccsc3)c12 ; COc1ccc(N2CCOCC2)cc1Nc1ncnc2[nH]nc(-c3ccsc3)c12]

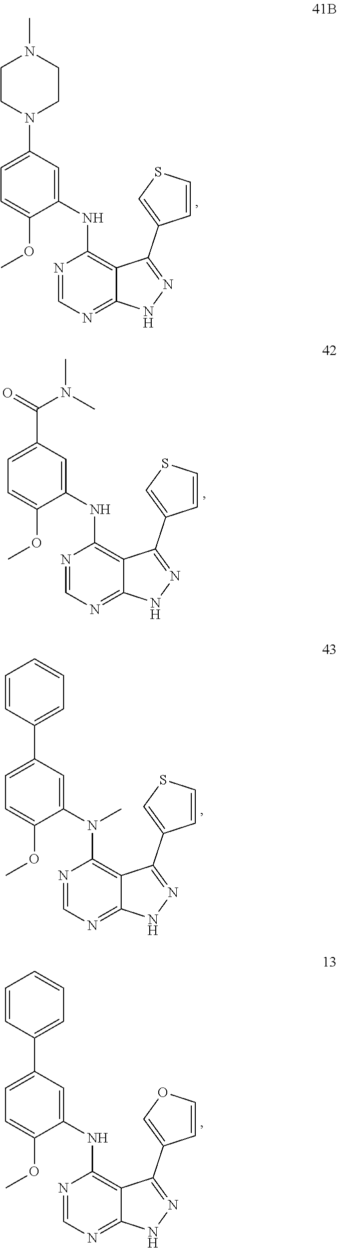 [SMILES: COc1ccc(N2CCN(C)CC2)cc1Nc1ncnc2[nH]nc(-c3ccsc3)c12 ; COc1ccc(C(=O)N(C)C)cc1Nc1ncnc2[nH]nc(-c3ccsc3)c12 ; COc1ccc(-c2ccccc2)cc1N(C)c1ncnc2[nH]nc(-c3ccsc3)c12 ; COc1ccc(-c2ccccc2)cc1Nc1ncnc2[nH]nc(-c3ccoc3)c12]

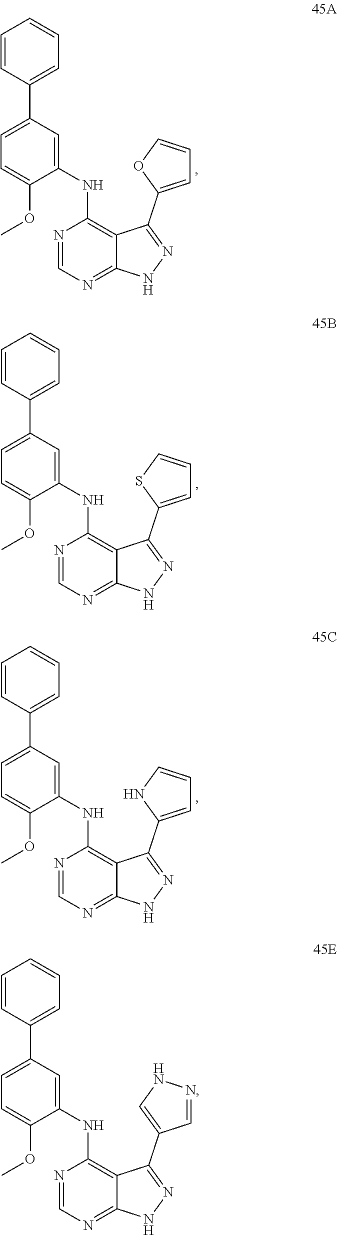 [SMILES: COc1ccc(-c2ccccc2)cc1Nc1ncnc2[nH]nc(-c3ccco3)c12 ; COc1ccc(-c2ccccc2)cc1Nc1ncnc2[nH]nc(-c3cccs3)c12 ; COc1ccc(-c2ccccc2)cc1Nc1ncnc2[nH]nc(-c3ccc[nH]3)c12 ; COc1ccc(-c2ccccc2)cc1Nc1ncnc2[nH]nc(-c3cn[nH]c3)c12]

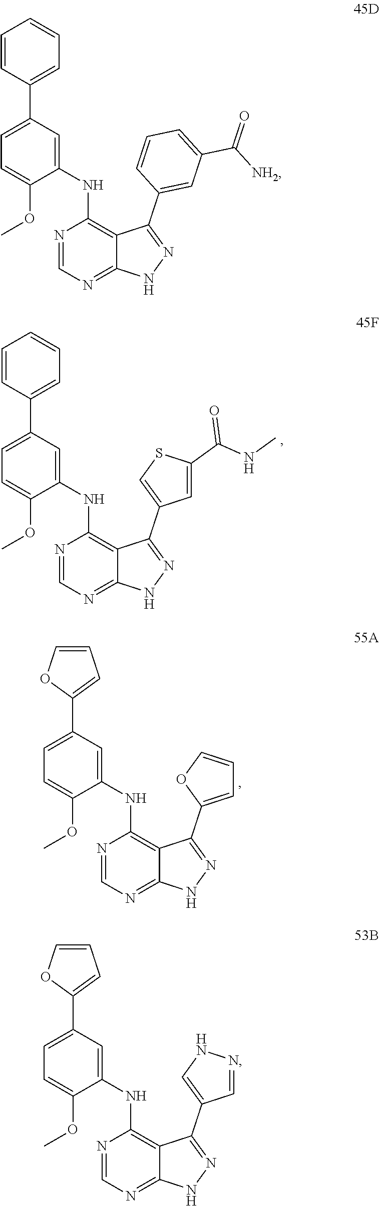 [SMILES: COc1ccc(-c2ccccc2)cc1Nc1ncnc2[nH]nc(-c3cccc(C(N)=O)c3)c12 ; CNC(=O)c1cc(-c2n[nH]c3ncnc(Nc4cc(-c5ccccc5)ccc4OC)c23)cs1 ; COc1ccc(-c2ccco2)cc1Nc1ncnc2[nH]nc(-c3ccco3)c12 ; COc1ccc(-c2ccco2)cc1Nc1ncnc2[nH]nc(-c3cn[nH]c3)c12]

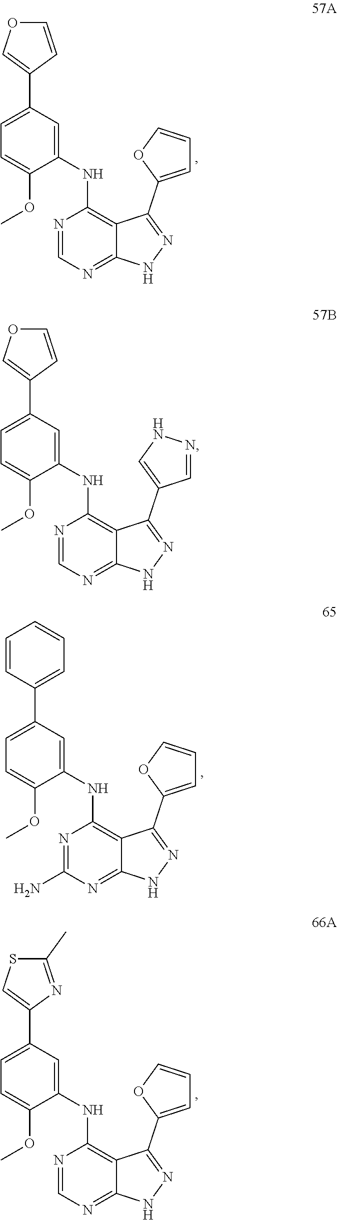 [SMILES: COc1ccc(-c2ccoc2)cc1Nc1ncnc2[nH]nc(-c3ccco3)c12 ; COc1ccc(-c2ccoc2)cc1Nc1ncnc2[nH]nc(-c3cn[nH]c3)c12 ; COc1ccc(-c2ccccc2)cc1Nc1nc(N)nc2[nH]nc(-c3ccco3)c12 ; COc1ccc(-c2csc(C)n2)cc1Nc1ncnc2[nH]nc(-c3ccco3)c12]

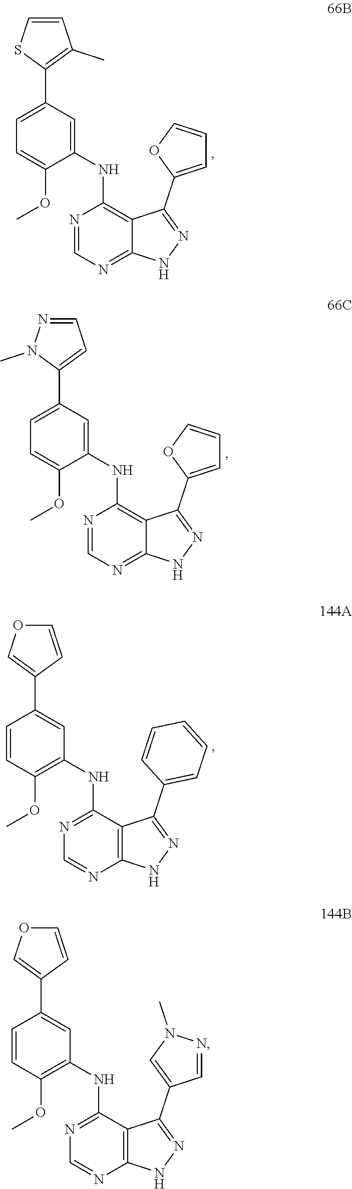 [SMILES: COc1ccc(-c2sccc2C)cc1Nc1ncnc2[nH]nc(-c3ccco3)c12 ; COc1ccc(-c2ccnn2C)cc1Nc1ncnc2[nH]nc(-c3ccco3)c12 ; COc1ccc(-c2ccoc2)cc1Nc1ncnc2[nH]nc(-c3ccccc3)c12 ; COc1ccc(-c2ccoc2)cc1Nc1ncnc2[nH]nc(-c3cnn(C)c3)c12]

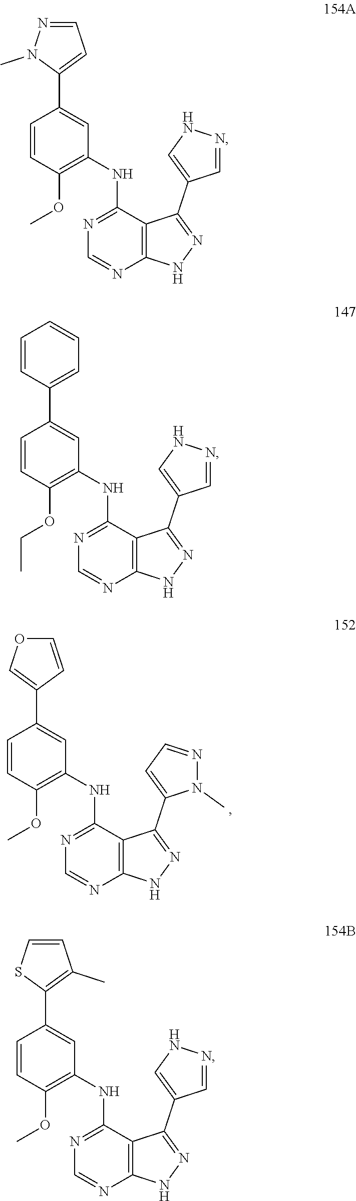 [SMILES: COc1ccc(-c2ccnn2C)cc1Nc1ncnc2[nH]nc(-c3cn[nH]c3)c12 ; CCOc1ccc(-c2ccccc2)cc1Nc1ncnc2[nH]nc(-c3cn[nH]c3)c12 ; COc1ccc(-c2ccoc2)cc1Nc1ncnc2[nH]nc(-c3ccnn3C)c12 ; COc1ccc(-c2sccc2C)cc1Nc1ncnc2[nH]nc(-c3cn[nH]c3)c12]

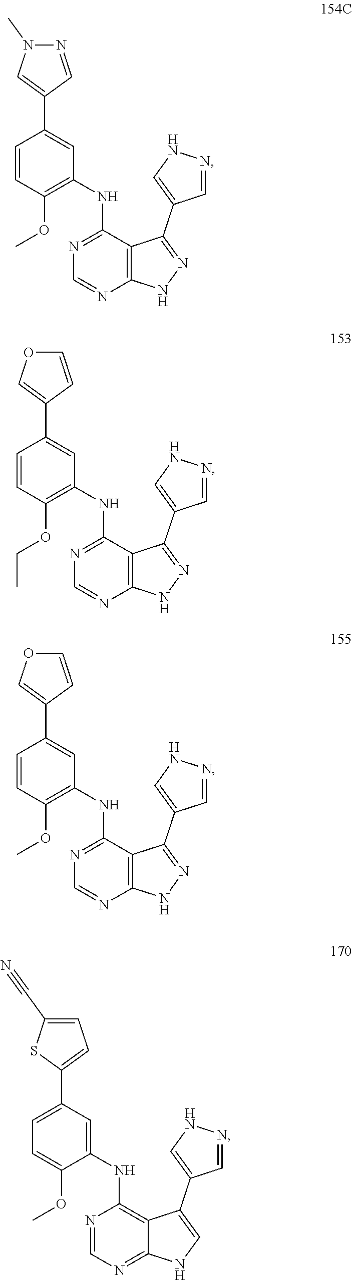 [SMILES: COc1ccc(-c2cnn(C)c2)cc1Nc1ncnc2[nH]nc(-c3cn[nH]c3)c12 ; CCOc1ccc(-c2ccoc2)cc1Nc1ncnc2[nH]nc(-c3cn[nH]c3)c12 ; COc1ccc(-c2ccoc2)cc1Nc1ncnc2[nH]nc(-c3cn[nH]c3)c12 ; COc1ccc(-c2ccc(C#N)s2)cc1Nc1ncnc2[nH]cc(-c3cn[nH]c3)c12]

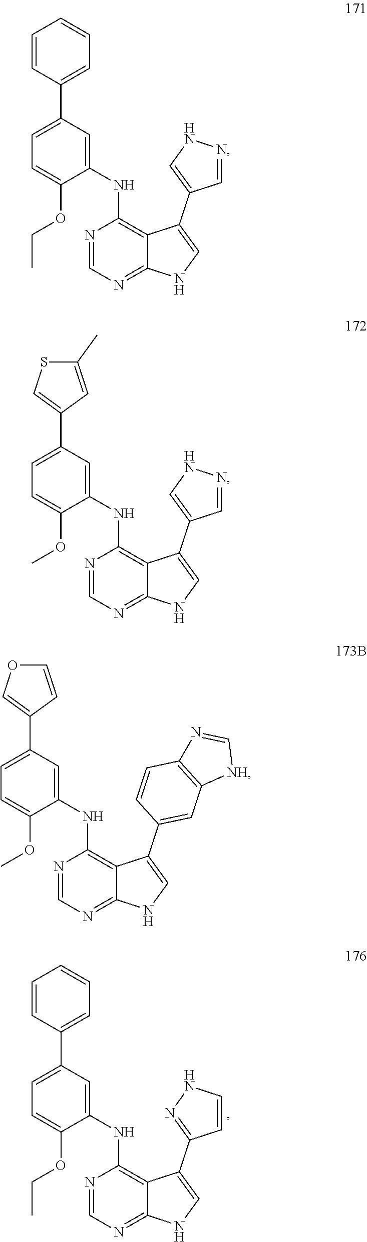 [SMILES: CCOc1ccc(-c2ccccc2)cc1Nc1ncnc2[nH]cc(-c3cn[nH]c3)c12 ; COc1ccc(-c2csc(C)c2)cc1Nc1ncnc2[nH]cc(-c3cn[nH]c3)c12 ; COc1ccc(-c2ccoc2)cc1Nc1ncnc2[nH]cc(-c3ccc4nc[nH]c4c3)c12 ; CCOc1ccc(-c2ccccc2)cc1Nc1ncnc2[nH]cc(-c3cc[nH]n3)c12]

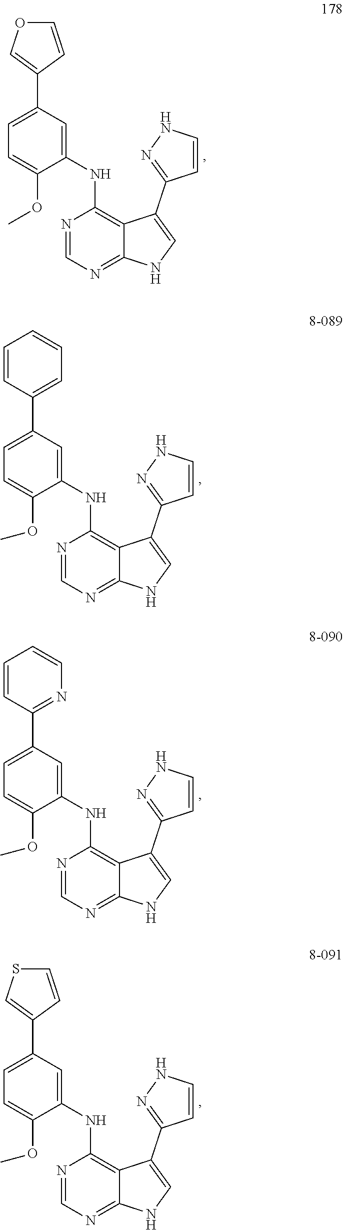 [SMILES: COc1ccc(-c2ccoc2)cc1Nc1ncnc2[nH]cc(-c3cc[nH]n3)c12 ; COc1ccc(-c2ccccc2)cc1Nc1ncnc2[nH]cc(-c3cc[nH]n3)c12 ; COc1ccc(-c2ccccn2)cc1Nc1ncnc2[nH]cc(-c3cc[nH]n3)c12 ; COc1ccc(-c2ccsc2)cc1Nc1ncnc2[nH]cc(-c3cc[nH]n3)c12]

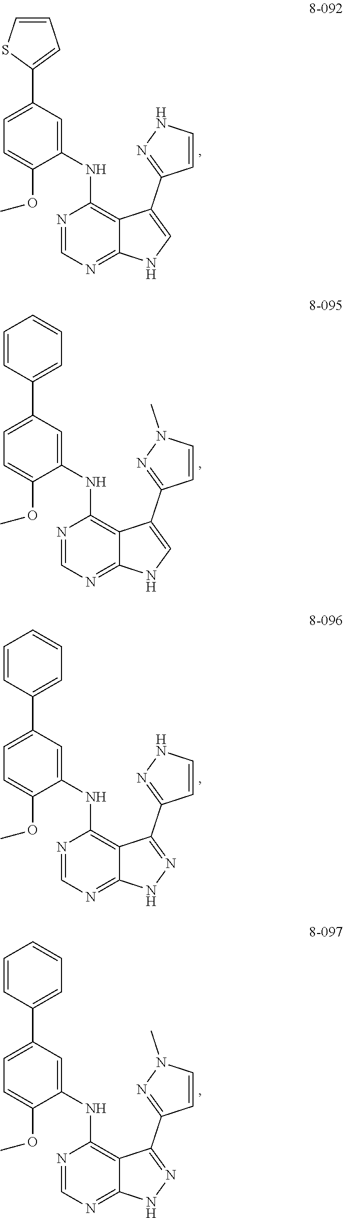 [SMILES: COc1ccc(-c2cccs2)cc1Nc1ncnc2[nH]cc(-c3cc[nH]n3)c12 ; COc1ccc(-c2ccccc2)cc1Nc1ncnc2[nH]cc(-c3ccn(C)n3)c12 ; COc1ccc(-c2ccccc2)cc1Nc1ncnc2[nH]nc(-c3cc[nH]n3)c12 ; COc1ccc(-c2ccccc2)cc1Nc1ncnc2[nH]nc(-c3ccn(C)n3)c12]

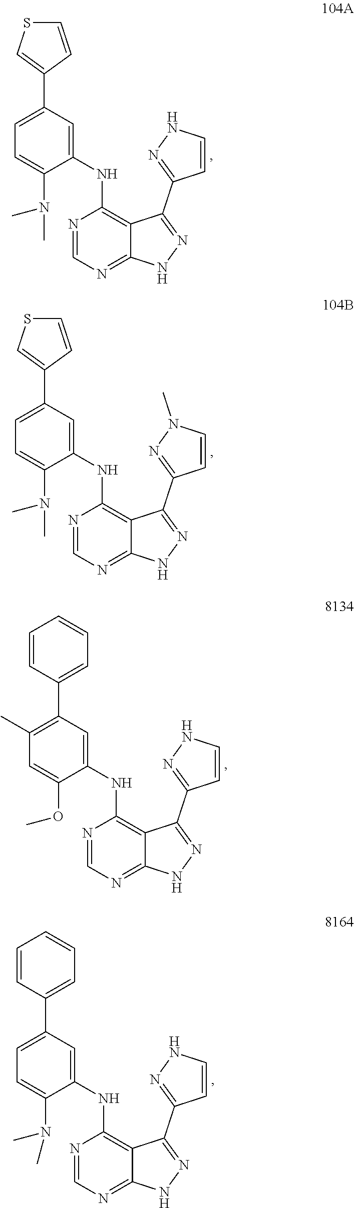 [SMILES: CN(C)c1ccc(-c2ccsc2)cc1Nc1ncnc2[nH]nc(-c3cc[nH]n3)c12 ; CN(C)c1ccc(-c2ccsc2)cc1Nc1ncnc2[nH]nc(-c3ccn(C)n3)c12 ; COc1cc(C)c(-c2ccccc2)cc1Nc1ncnc2[nH]nc(-c3cc[nH]n3)c12 ; CN(C)c1ccc(-c2ccccc2)cc1Nc1ncnc2[nH]nc(-c3cc[nH]n3)c12]

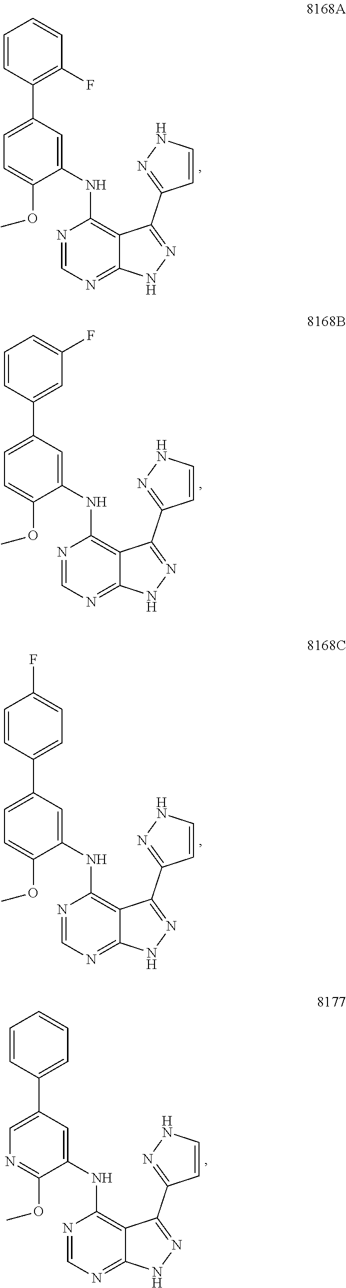 [SMILES: COc1ccc(-c2ccccc2F)cc1Nc1ncnc2[nH]nc(-c3cc[nH]n3)c12 ; COc1ccc(-c2cccc(F)c2)cc1Nc1ncnc2[nH]nc(-c3cc[nH]n3)c12 ; COc1ccc(-c2ccc(F)cc2)cc1Nc1ncnc2[nH]nc(-c3cc[nH]n3)c12 ; COc1ncc(-c2ccccc2)cc1Nc1ncnc2[nH]nc(-c3cc[nH]n3)c12]

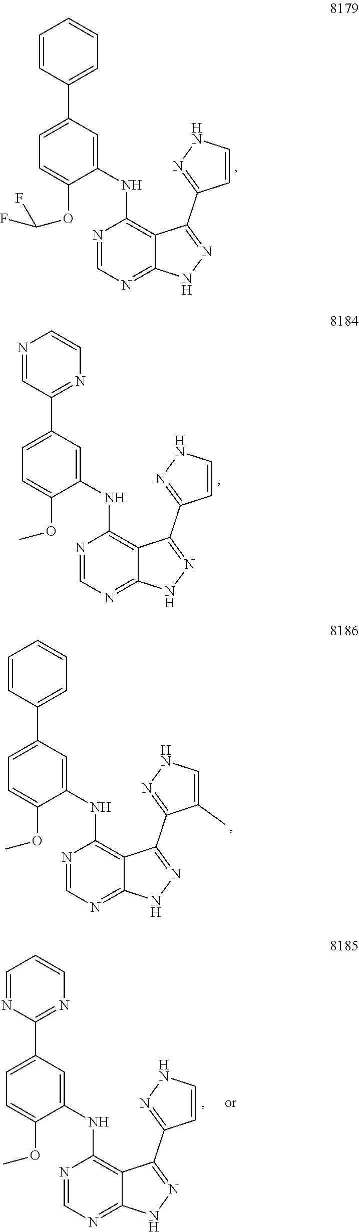

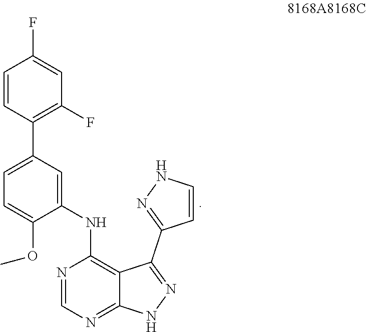 [SMILES: COc1ccc(-c2ccc(F)cc2F)cc1Nc1ncnc2[nH]nc(-c3cc[nH]n3)c12]

In embodiments, the compound has the formula

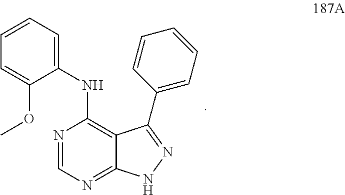 [SMILES: COc1ccccc1Nc1ncnc2[nH]nc(-c3ccccc3)c12]

In embodiments, the compound has the formula:

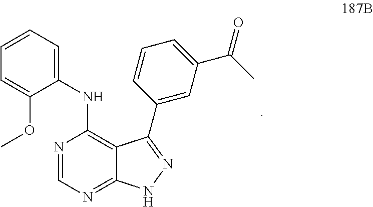 [SMILES: COc1ccccc1Nc1ncnc2[nH]nc(-c3cccc(C(C)=O)c3)c12]

In embodiments, the compound has the formula:

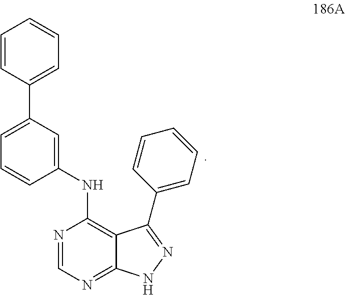 [SMILES: c1ccc(-c2cccc(Nc3ncnc4[nH]nc(-c5ccccc5)c34)c2)cc1]

In embodiments, the compound has the formula:

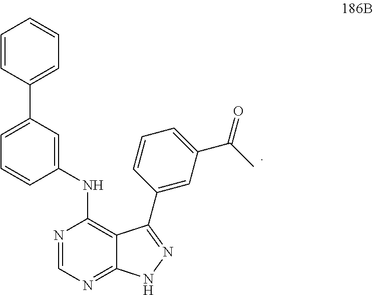 [SMILES: CC(=O)c1cccc(-c2n[nH]c3ncnc(Nc4cccc(-c5ccccc5)c4)c23)c1]

In embodiments, the compound has the formula:

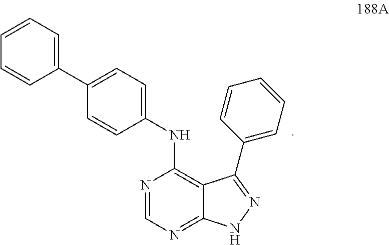 [SMILES: c1ccc(-c2ccc(Nc3ncnc4[nH]nc(-c5ccccc5)c34)cc2)cc1]

In embodiments, the compound has the formula:

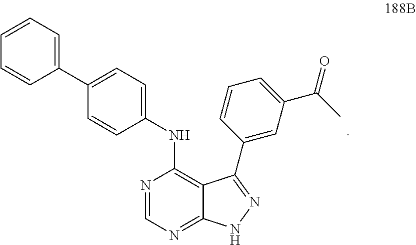 [SMILES: CC(=O)c1cccc(-c2n[nH]c3ncnc(Nc4ccc(-c5ccccc5)cc4)c23)c1]

In embodiments, the compound has the formula:

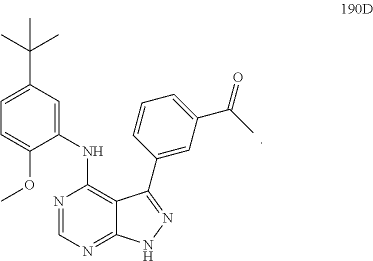 [SMILES: COc1ccc(C(C)(C)C)cc1Nc1ncnc2[nH]nc(-c3cccc(C(C)=O)c3)c12]

In embodiments, the compound has the formula:

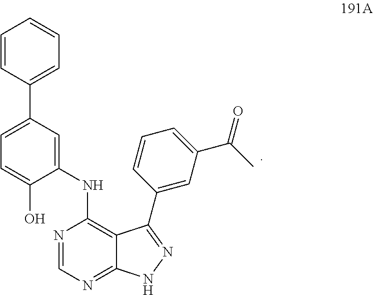 [SMILES: CC(=O)c1cccc(-c2n[nH]c3ncnc(Nc4cc(-c5ccccc5)ccc4O)c23)c1]

In embodiments, the compound has the formula:

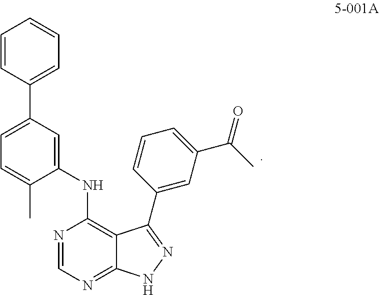 [SMILES: CC(=O)c1cccc(-c2n[nH]c3ncnc(Nc4cc(-c5ccccc5)ccc4C)c23)c1]

In embodiments, the compound has the formula:

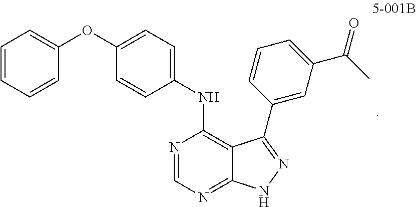 [SMILES: CC(=O)c1cccc(-c2n[nH]c3ncnc(Nc4ccc(Oc5ccccc5)cc4)c23)c1]

In embodiments, the compound has the formula:

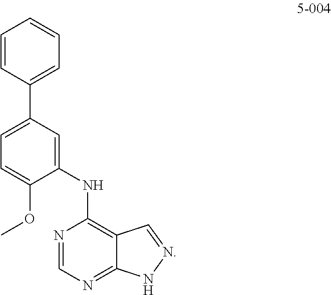 [SMILES: COc1ccc(-c2ccccc2)cc1Nc1ncnc2[nH]ncc12]

In embodiments, the compound has the formula:

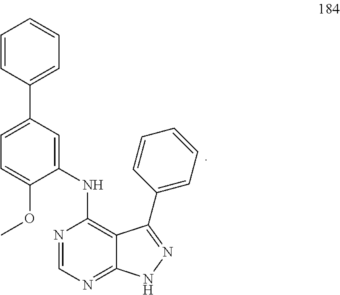 [SMILES: COc1ccc(-c2ccccc2)cc1Nc1ncnc2[nH]nc(-c3ccccc3)c12]

In embodiments, the compound has the formula:

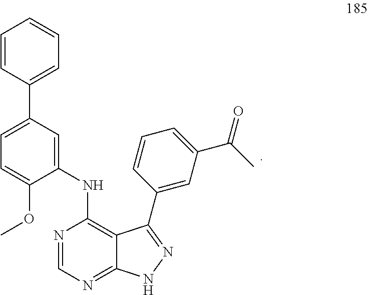 [SMILES: COc1ccc(-c2ccccc2)cc1Nc1ncnc2[nH]nc(-c3cccc(C(C)=O)c3)c12]

In embodiments, the compound has the formula:

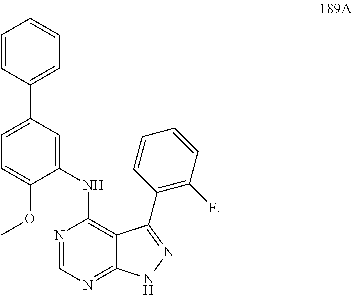 [SMILES: COc1ccc(-c2ccccc2)cc1Nc1ncnc2[nH]nc(-c3ccccc3F)c12]

In embodiments, the compound has the formula:

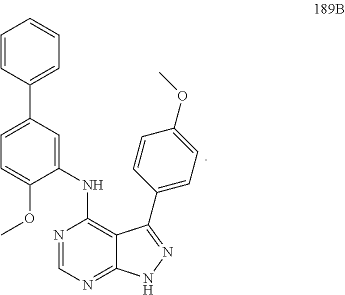 [SMILES: COc1ccc(-c2n[nH]c3ncnc(Nc4cc(-c5ccccc5)ccc4OC)c23)cc1]

In embodiments, the compound has the formula:

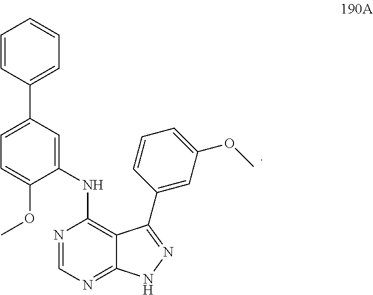 [SMILES: COc1cccc(-c2n[nH]c3ncnc(Nc4cc(-c5ccccc5)ccc4OC)c23)c1]

In embodiments, the compound has the formula:

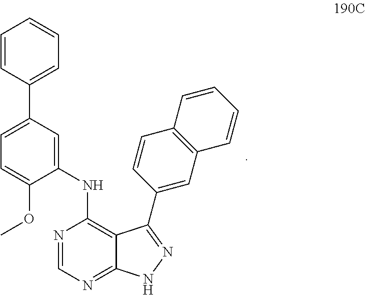 [SMILES: COc1ccc(-c2ccccc2)cc1Nc1ncnc2[nH]nc(-c3ccc4ccccc4c3)c12]

In embodiments, the compound has the formula:

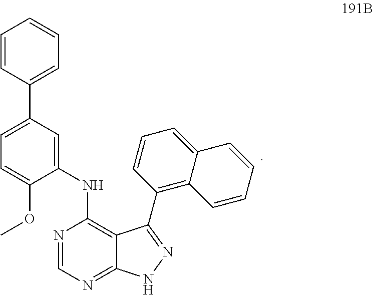 [SMILES: COc1ccc(-c2ccccc2)cc1Nc1ncnc2[nH]nc(-c3cccc4ccccc34)c12]

In embodiments, the compound has the formula:

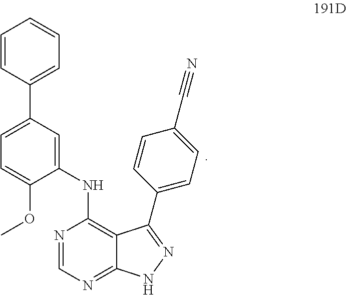 [SMILES: COc1ccc(-c2ccccc2)cc1Nc1ncnc2[nH]nc(-c3ccc(C#N)cc3)c12]

In embodiments, the compound has the formula:

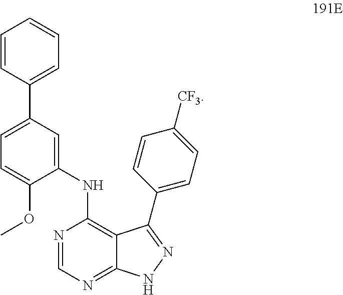 [SMILES: COc1ccc(-c2ccccc2)cc1Nc1ncnc2[nH]nc(-c3ccc(C(F)(F)F)cc3)c12]

In embodiments, the compound has the formula:

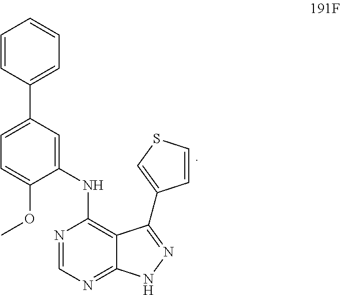 [SMILES: COc1ccc(-c2ccccc2)cc1Nc1ncnc2[nH]nc(-c3ccsc3)c12]

In embodiments, the compound has the formula:

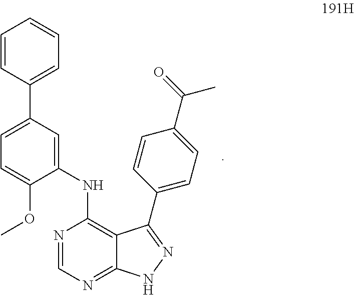 [SMILES: COc1ccc(-c2ccccc2)cc1Nc1ncnc2[nH]nc(-c3ccc(C(C)=O)cc3)c12]

In embodiments, the compound has the formula:

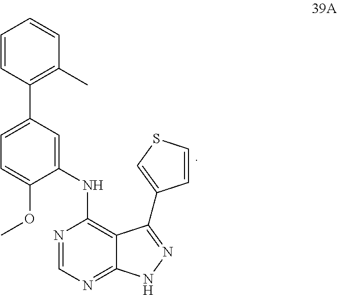 [SMILES: COc1ccc(-c2ccccc2C)cc1Nc1ncnc2[nH]nc(-c3ccsc3)c12]

In embodiments, the compound has the formula:

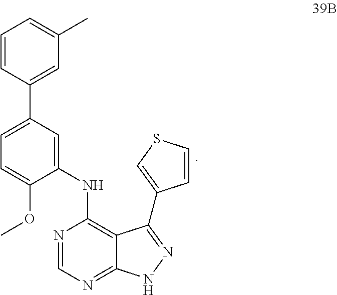 [SMILES: COc1ccc(-c2cccc(C)c2)cc1Nc1ncnc2[nH]nc(-c3ccsc3)c12]

In embodiments, the compound has the formula:

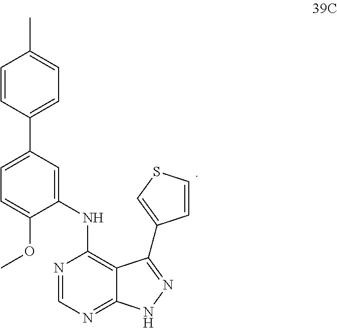 [SMILES: COc1ccc(-c2ccc(C)cc2)cc1Nc1ncnc2[nH]nc(-c3ccsc3)c12]

In embodiments, the compound has the formula:

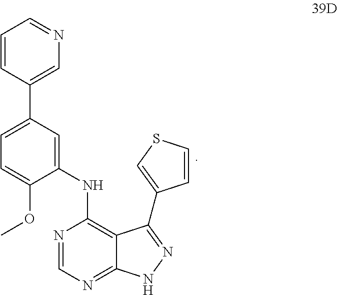 [SMILES: COc1ccc(-c2cccnc2)cc1Nc1ncnc2[nH]nc(-c3ccsc3)c12]

In embodiments, the compound has the formula:

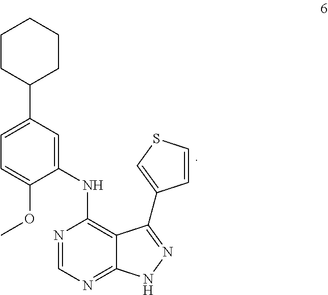 [SMILES: COc1ccc(C2CCCCC2)cc1Nc1ncnc2[nH]nc(-c3ccsc3)c12]

In embodiments, the compound has the formula:

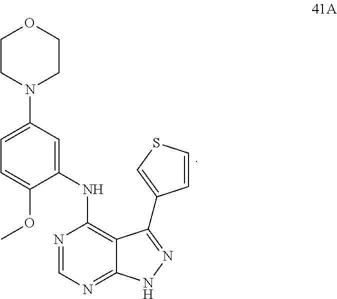 [SMILES: COc1ccc(N2CCOCC2)cc1Nc1ncnc2[nH]nc(-c3ccsc3)c12]

In embodiments, the compound has the formula:

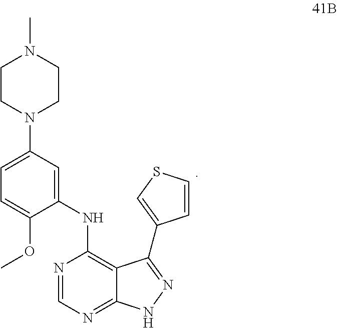 [SMILES: COc1ccc(N2CCN(C)CC2)cc1Nc1ncnc2[nH]nc(-c3ccsc3)c12]

In embodiments, the compound has the formula:

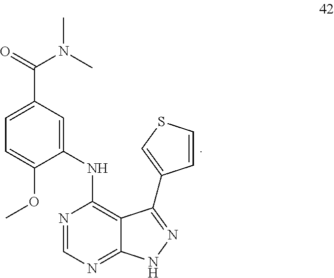 [SMILES: COc1ccc(C(=O)N(C)C)cc1Nc1ncnc2[nH]nc(-c3ccsc3)c12]

In embodiments, the compound has the formula:

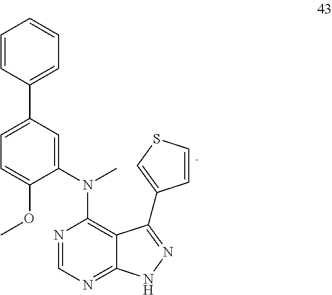 [SMILES: COc1ccc(-c2ccccc2)cc1N(C)c1ncnc2[nH]nc(-c3ccsc3)c12]

In embodiments, the compound has the formula:

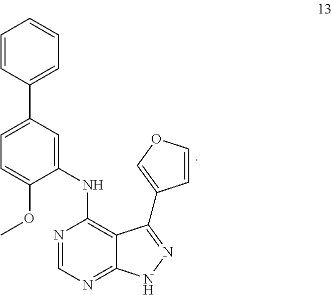 [SMILES: COc1ccc(-c2ccccc2)cc1Nc1ncnc2[nH]nc(-c3ccoc3)c12]

In embodiments, the compound has the formula:

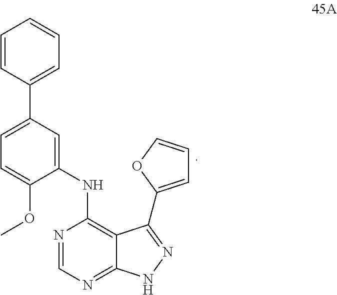 [SMILES: COc1ccc(-c2ccccc2)cc1Nc1ncnc2[nH]nc(-c3ccco3)c12]

In embodiments, the compound has the formula:

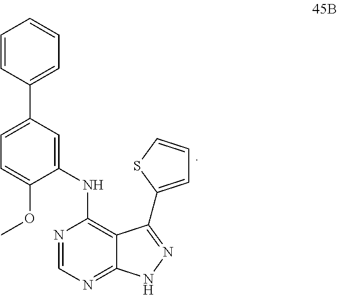 [SMILES: COc1ccc(-c2ccccc2)cc1Nc1ncnc2[nH]nc(-c3cccs3)c12]

In embodiments, the compound has the formula:

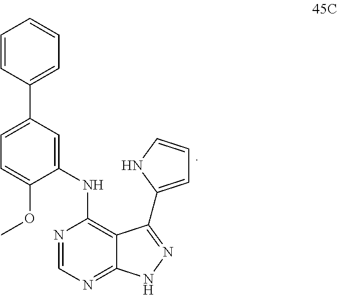 [SMILES: COc1ccc(-c2ccccc2)cc1Nc1ncnc2[nH]nc(-c3ccc[nH]3)c12]

In embodiments, the compound has the formula:

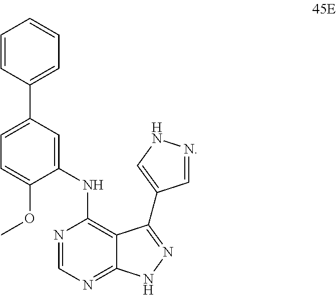 [SMILES: COc1ccc(-c2ccccc2)cc1Nc1ncnc2[nH]nc(-c3cn[nH]c3)c12]

In embodiments, the compound has the formula:

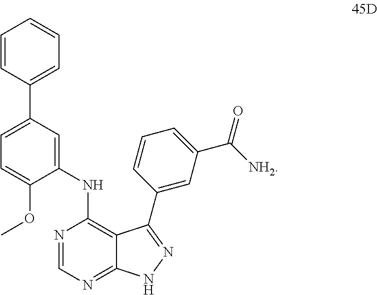 [SMILES: COc1ccc(-c2ccccc2)cc1Nc1ncnc2[nH]nc(-c3cccc(C(N)=O)c3)c12]

In embodiments, the compound has the formula:

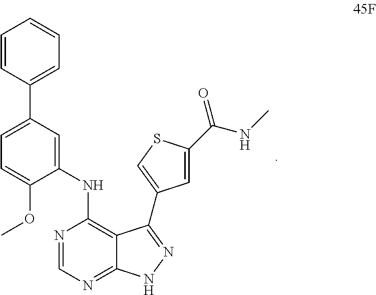 [SMILES: CNC(=O)c1cc(-c2n[nH]c3ncnc(Nc4cc(-c5ccccc5)ccc4OC)c23)cs1]

In embodiments, the compound has the formula:

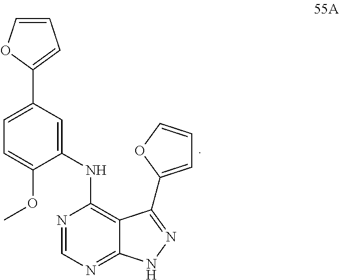 [SMILES: COc1ccc(-c2ccco2)cc1Nc1ncnc2[nH]nc(-c3ccco3)c12]

In embodiments, the compound has the formula:

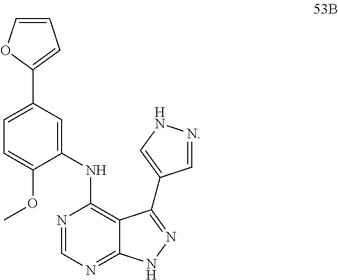 [SMILES: COc1ccc(-c2ccco2)cc1Nc1ncnc2[nH]nc(-c3cn[nH]c3)c12]

In embodiments, the compound has the formula:

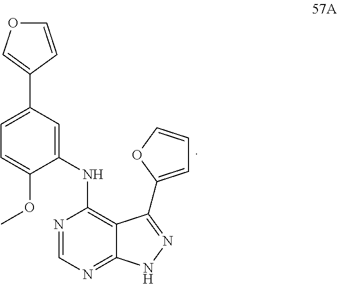 [SMILES: COc1ccc(-c2ccoc2)cc1Nc1ncnc2[nH]nc(-c3ccco3)c12]

In embodiments, the compound has the formula:

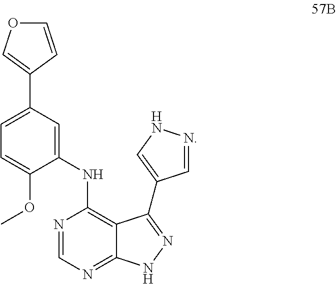 [SMILES: COc1ccc(-c2ccoc2)cc1Nc1ncnc2[nH]nc(-c3cn[nH]c3)c12]

In embodiments, the compound has the formula:

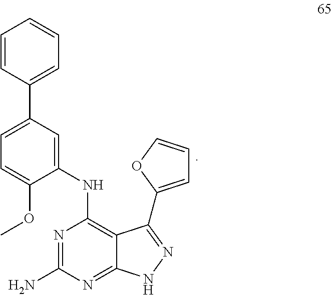 [SMILES: COc1ccc(-c2ccccc2)cc1Nc1nc(N)nc2[nH]nc(-c3ccco3)c12]

In embodiments, the compound has the formula:

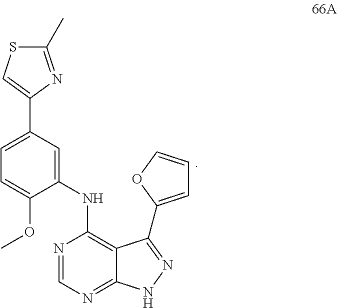 [SMILES: COc1ccc(-c2csc(C)n2)cc1Nc1ncnc2[nH]nc(-c3ccco3)c12]

In embodiments, the compound has the formula:

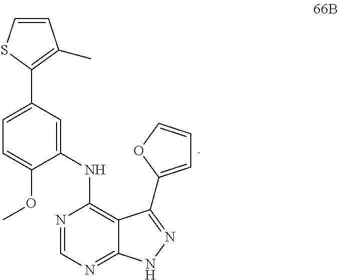 [SMILES: COc1ccc(-c2sccc2C)cc1Nc1ncnc2[nH]nc(-c3ccco3)c12]

In embodiments, the compound has the formula:

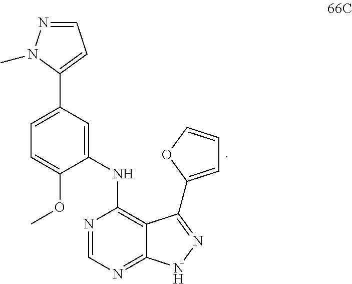 [SMILES: COc1ccc(-c2ccnn2C)cc1Nc1ncnc2[nH]nc(-c3ccco3)c12]

In embodiments, the compound has the formula:

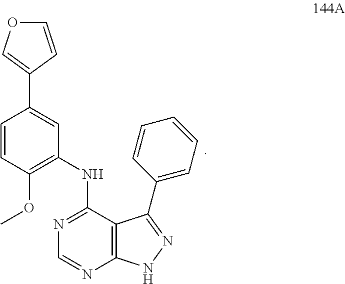 [SMILES: COc1ccc(-c2ccoc2)cc1Nc1ncnc2[nH]nc(-c3ccccc3)c12]

In embodiments, the compound has the formula:

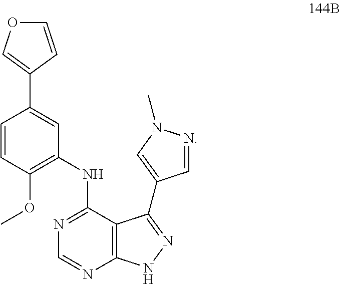 [SMILES: COc1ccc(-c2ccoc2)cc1Nc1ncnc2[nH]nc(-c3cnn(C)c3)c12]

In embodiments, the compound has the formula:

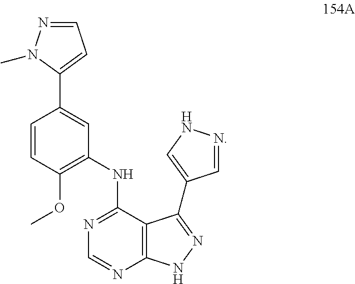 [SMILES: COc1ccc(-c2ccnn2C)cc1Nc1ncnc2[nH]nc(-c3cn[nH]c3)c12]

In embodiments, the compound has the formula:

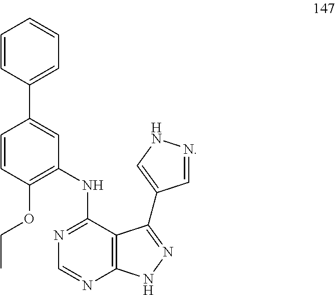 [SMILES: CCOc1ccc(-c2ccccc2)cc1Nc1ncnc2[nH]nc(-c3cn[nH]c3)c12]

In embodiments, the compound has the formula:

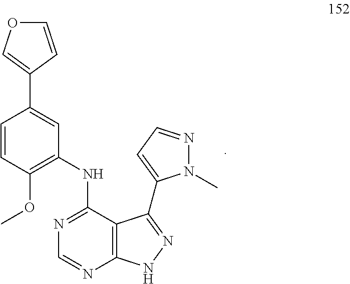

In embodiments, the compound has the formula:

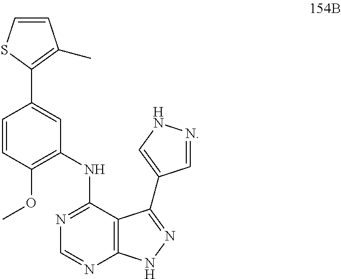 [SMILES: COc1ccc(-c2sccc2C)cc1Nc1ncnc2[nH]nc(-c3cn[nH]c3)c12]

In embodiments, the compound has the formula:

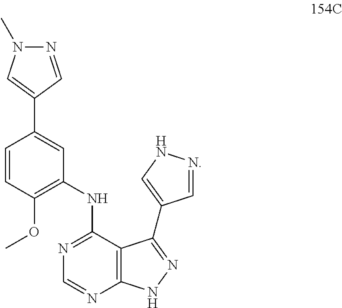 [SMILES: COc1ccc(-c2cnn(C)c2)cc1Nc1ncnc2[nH]nc(-c3cn[nH]c3)c12]

In embodiments, the compound has the formula:

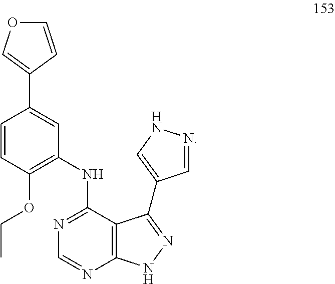 [SMILES: CCOc1ccc(-c2ccoc2)cc1Nc1ncnc2[nH]nc(-c3cn[nH]c3)c12]

In embodiments, the compound has the formula:

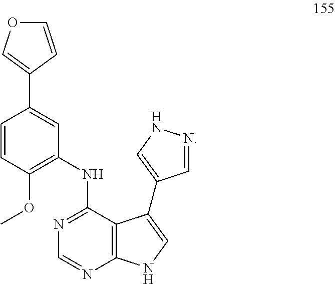 [SMILES: COc1ccc(-c2ccoc2)cc1Nc1ncnc2[nH]cc(-c3cn[nH]c3)c12]

In embodiments, the compound has the formula:

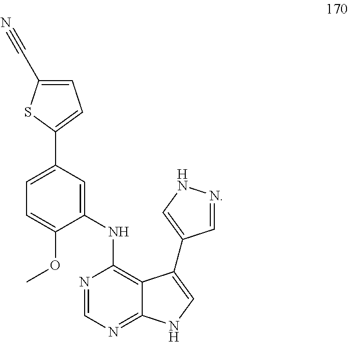 [SMILES: COc1ccc(-c2ccc(C#N)s2)cc1Nc1ncnc2[nH]cc(-c3cn[nH]c3)c12]

In embodiments, the compound has the formula:

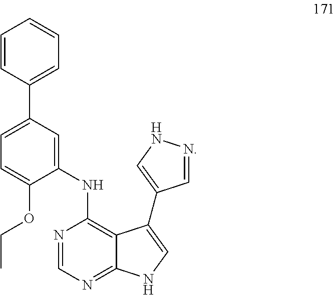 [SMILES: CCOc1ccc(-c2ccccc2)cc1Nc1ncnc2[nH]cc(-c3cn[nH]c3)c12]

In embodiments, the compound has the formula:

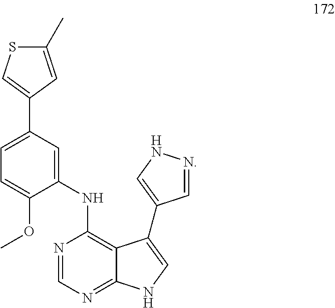 [SMILES: COc1ccc(-c2csc(C)c2)cc1Nc1ncnc2[nH]cc(-c3cn[nH]c3)c12]

In embodiments, the compound has the formula:

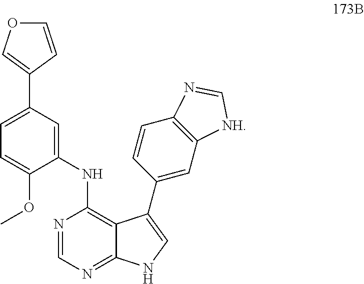 [SMILES: COc1ccc(-c2ccoc2)cc1Nc1ncnc2[nH]cc(-c3ccc4nc[nH]c4c3)c12]

In embodiments, the compound has the formula:

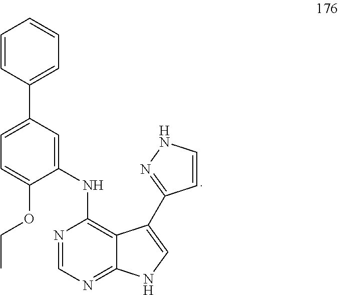 [SMILES: CCOc1ccc(-c2ccccc2)cc1Nc1ncnc2[nH]cc(-c3cc[nH]n3)c12 ; "."]

In embodiments, the compound has the formula:

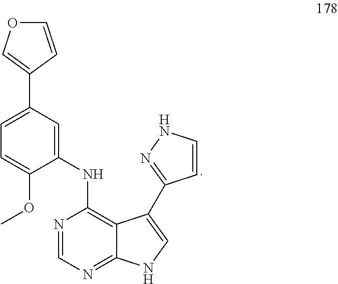 [SMILES: COc1ccc(-c2ccoc2)cc1Nc1ncnc2[nH]cc(-c3cc[nH]n3)c12 ; "."]

In embodiments, the compound has the formula:

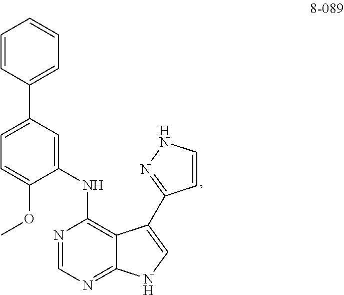 [SMILES: COc1ccc(-c2ccccc2)cc1Nc1ncnc2[nH]cc(-c3cc[nH]n3)c12 ; ","]

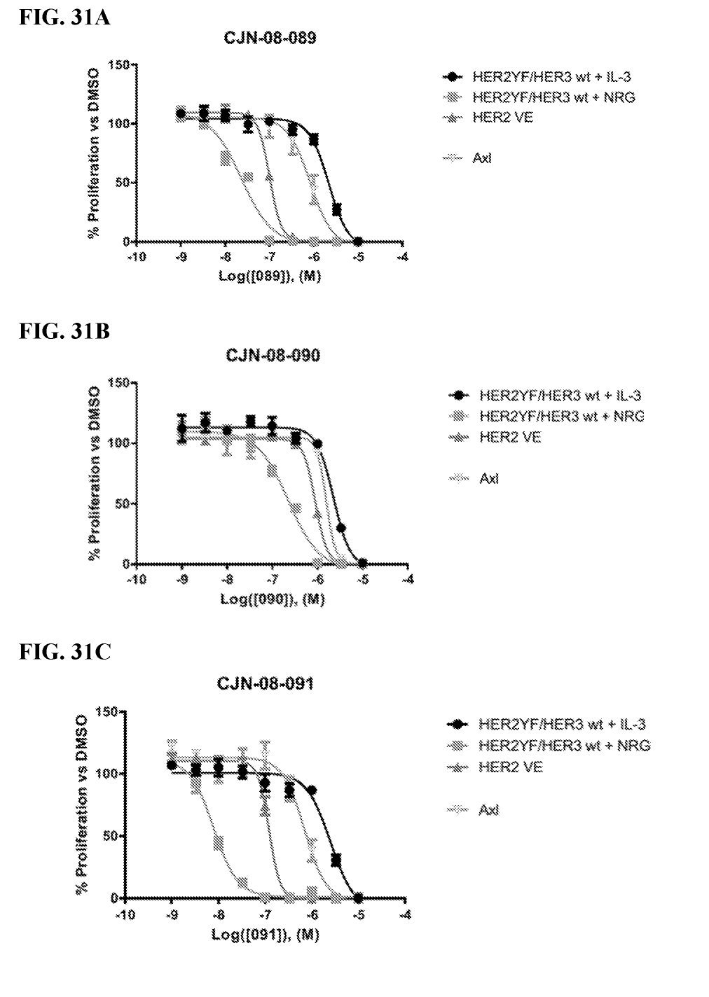

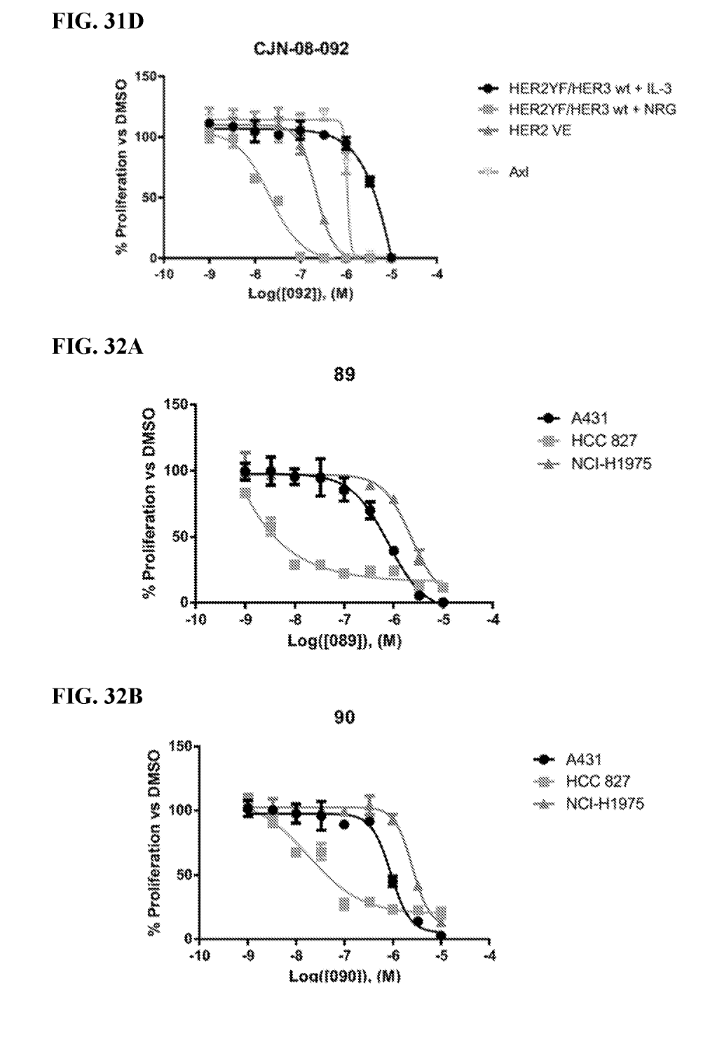

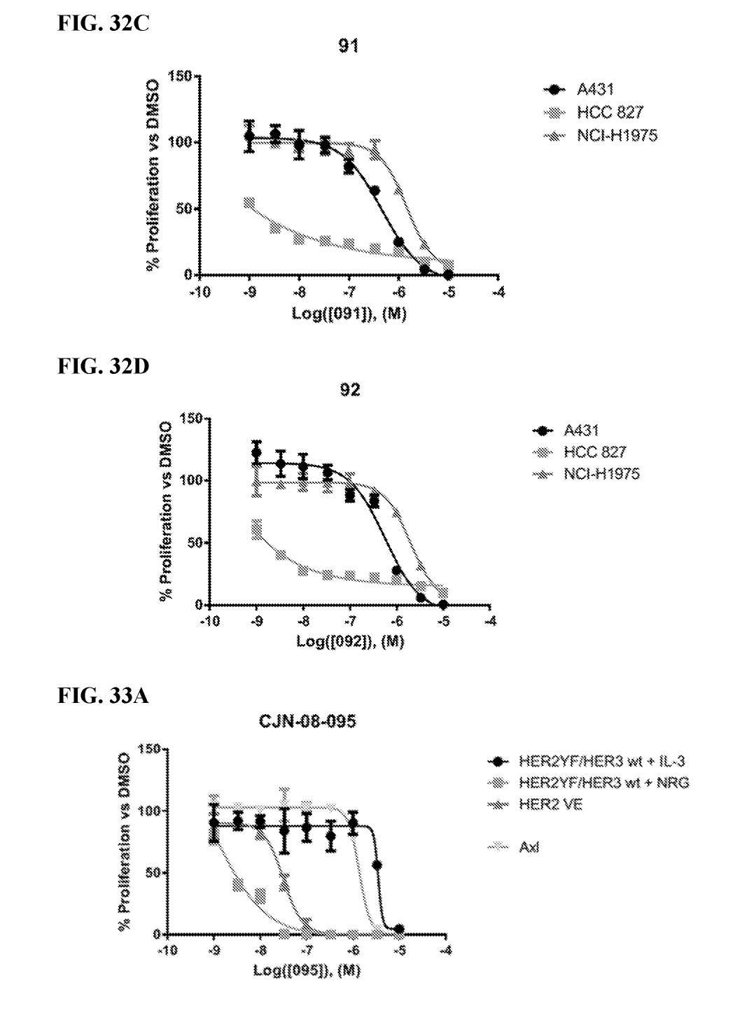

also referred to as CJN-08-089 in FIG. 31A.

In embodiments, the compound has the formula:

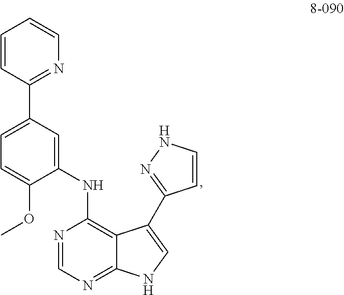 [SMILES: COc1ccc(-c2ccccn2)cc1Nc1ncnc2[nH]cc(-c3cc[nH]n3)c12 ; ","]

also referred to herein as CJN-08-090 as in FIG. 31B.

In embodiments, the compound has the formula:

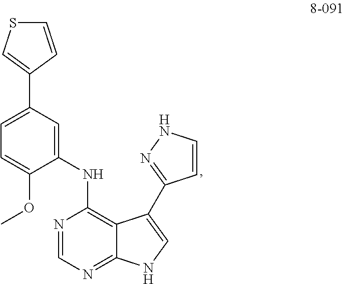 [SMILES: COc1ccc(-c2ccsc2)cc1Nc1ncnc2[nH]cc(-c3cc[nH]n3)c12 ; ","]

also referred to herein as CJN-08-091 as in FIG. 31C.

In embodiments, the compound has the formula:

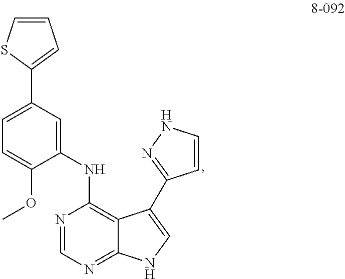 [SMILES: COc1ccc(-c2cccs2)cc1Nc1ncnc2[nH]cc(-c3cc[nH]n3)c12 ; ","]

also referred to herein as CJN-08-092 as in FIG. 31D.

In embodiments, the compound has the formula:

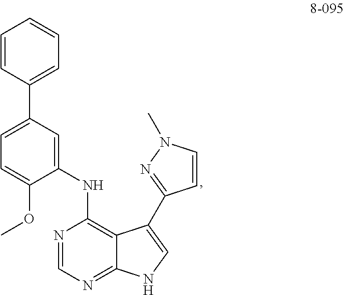 [SMILES: COc1ccc(-c2ccccc2)cc1Nc1ncnc2[nH]cc(-c3ccn(C)n3)c12 ; ","]

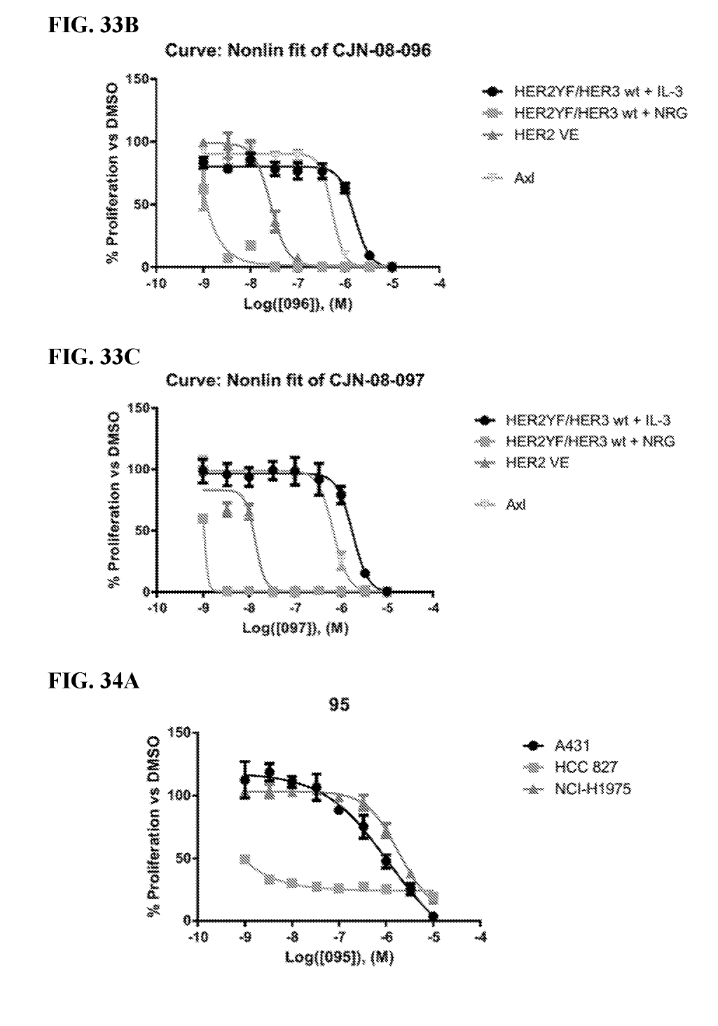

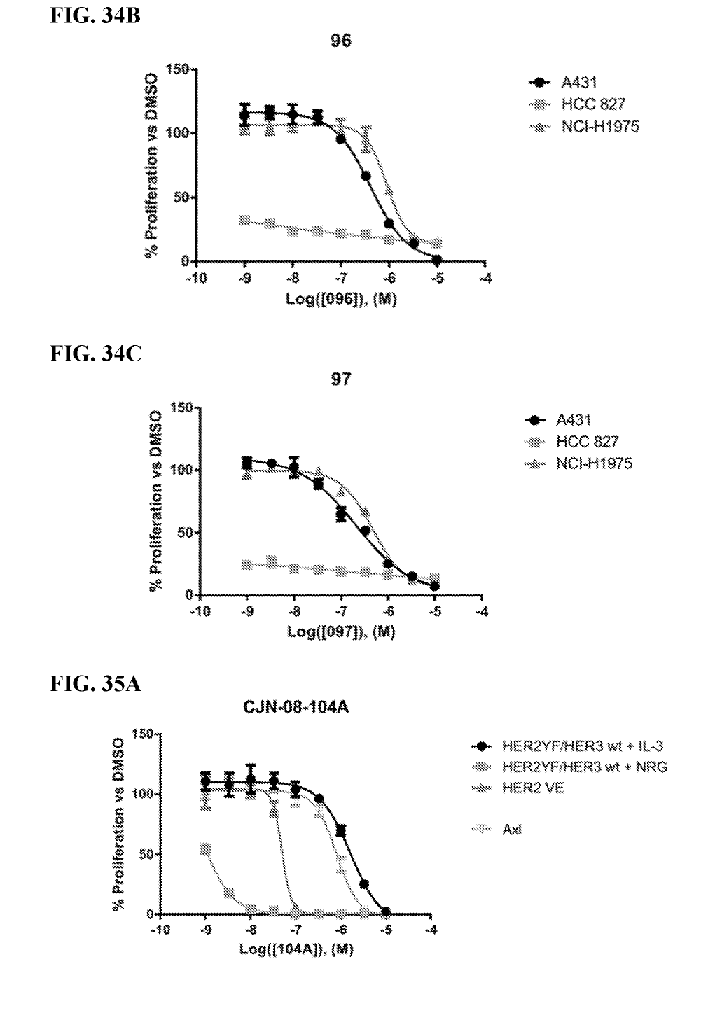

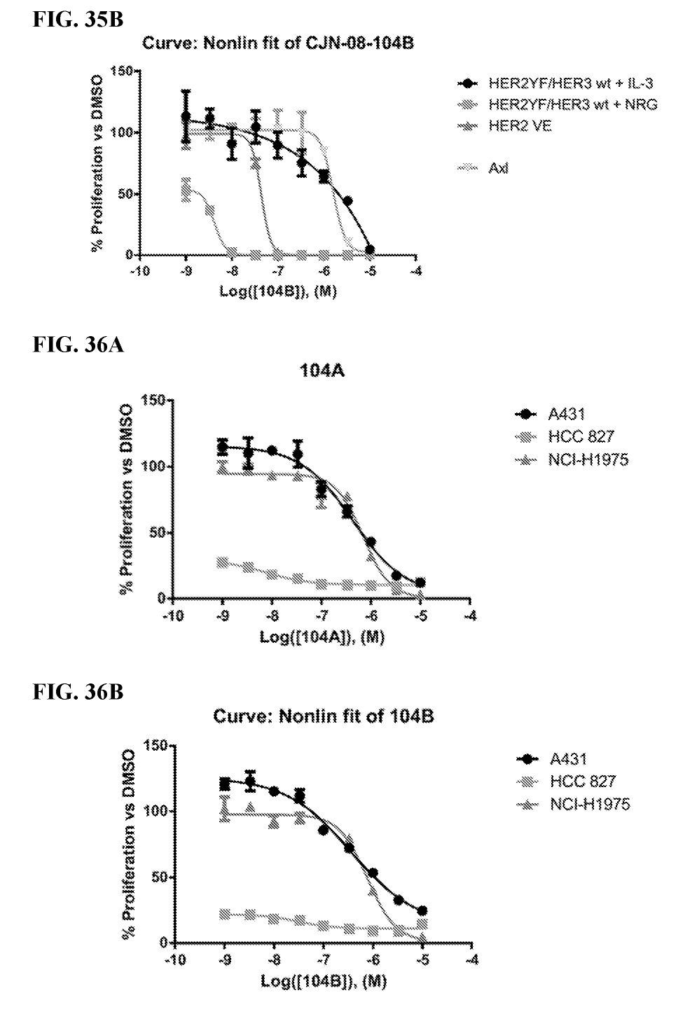

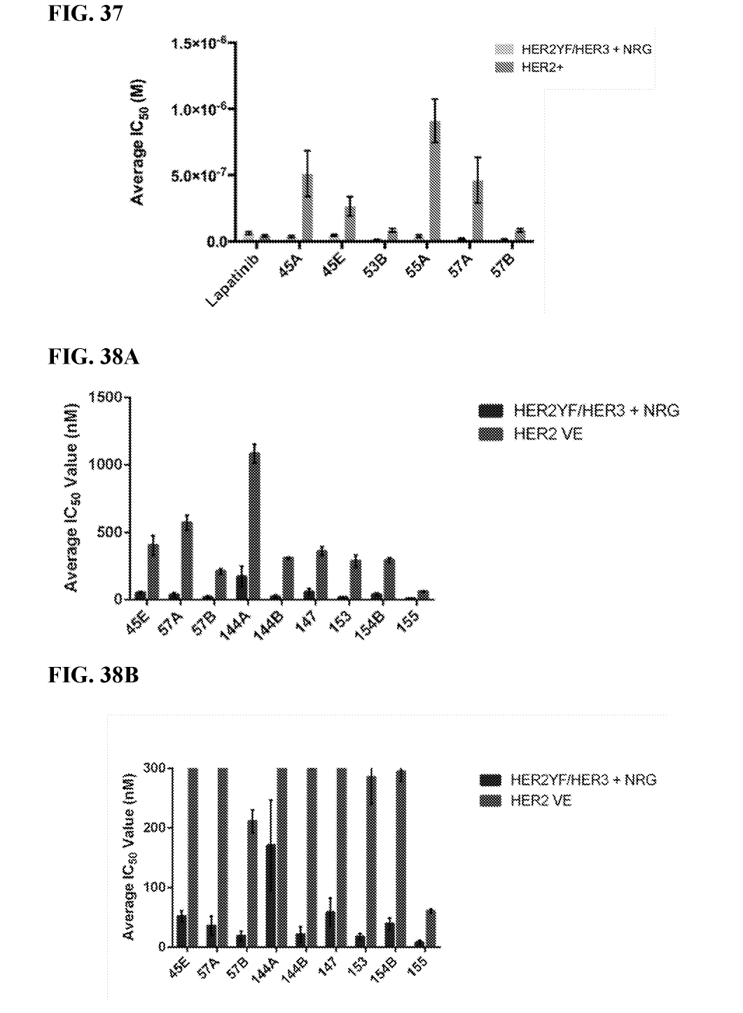

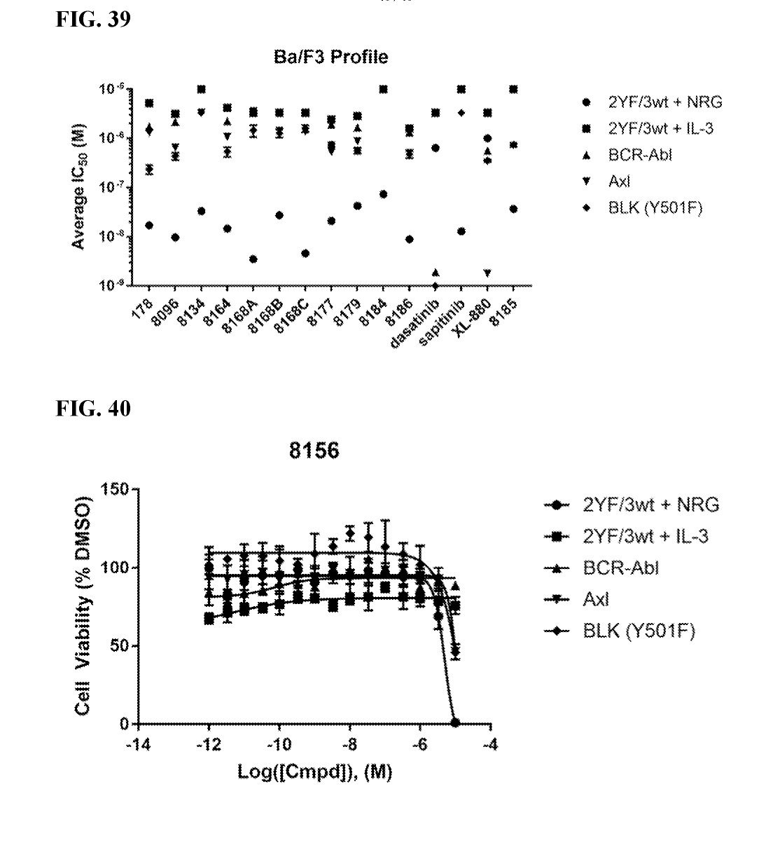

also referred to herein as CJN-08-0985 as in FIG. 33A.

In embodiments, the compound has the formula:

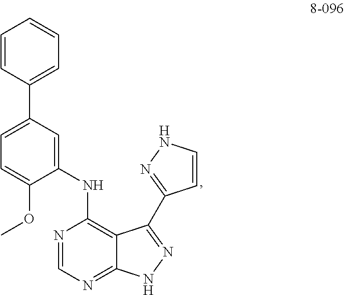 [SMILES: COc1ccc(-c2ccccc2)cc1Nc1ncnc2[nH]nc(-c3cc[nH]n3)c12 ; ","]

also referred to herein as CJN-08-096 as in FIG. 33B.

In embodiments, the compound has the formula:

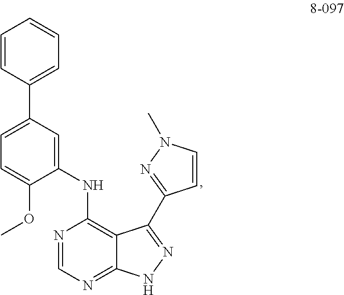 [SMILES: COc1ccc(-c2ccccc2)cc1Nc1ncnc2[nH]nc(-c3ccn(C)n3)c12 ; ","]

also referred to herein as CJN-08-097 as in FIG. 33C.

In embodiments, the compound has the formula:

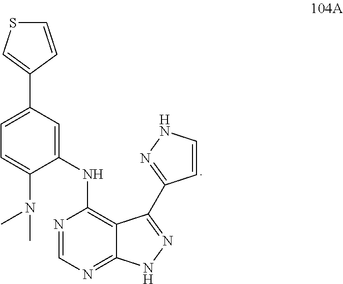 [SMILES: CN(C)c1ccc(-c2ccsc2)cc1Nc1ncnc2[nH]nc(-c3cc[nH]n3)c12 ; "."]

In embodiments, the compound has the formula:

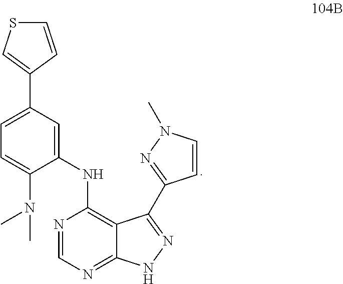 [SMILES: CN(C)c1ccc(-c2ccsc2)cc1Nc1ncnc2[nH]nc(-c3ccn(C)n3)c12 ; "."]

In embodiments, the compound has the formula

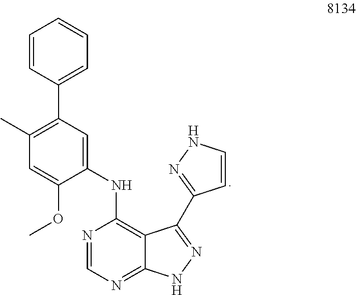 [SMILES: COc1cc(C)c(-c2ccccc2)cc1Nc1ncnc2[nH]nc(-c3cc[nH]n3)c12 ; "."]

In embodiments, the compound has the formula:

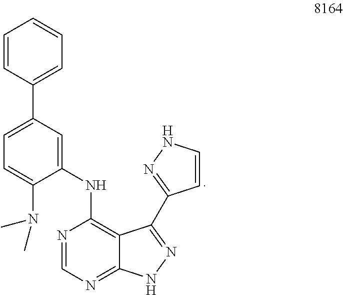 [SMILES: CN(C)c1ccc(-c2ccccc2)cc1Nc1ncnc2[nH]nc(-c3cc[nH]n3)c12 ; "."]

In embodiments, the compound has the formula:

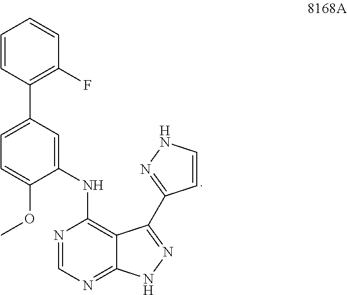 [SMILES: COc1ccc(-c2ccccc2F)cc1Nc1ncnc2[nH]nc(-c3cc[nH]n3)c12 ; "."]

In embodiments, the compound has the formula:

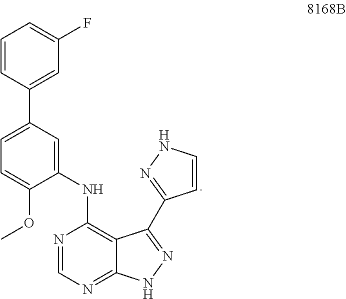 [SMILES: COc1ccc(-c2cccc(F)c2)cc1Nc1ncnc2[nH]nc(-c3cc[nH]n3)c12 ; "."]

In embodiments, the compound has the formula:

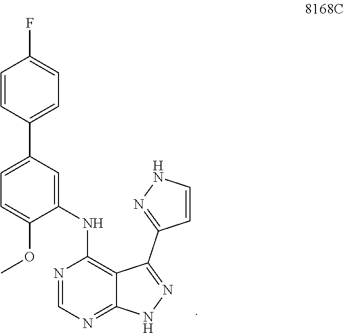 [SMILES: COc1ccc(-c2ccc(F)cc2)cc1Nc1ncnc2[nH]nc(-c3cc[nH]n3)c12]

In embodiments, the compound has the formula:

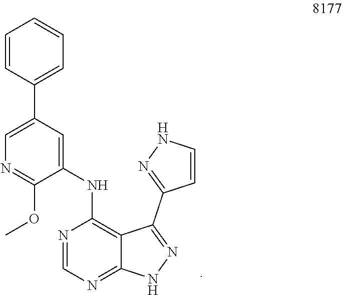 [SMILES: COc1ncc(-c2ccccc2)cc1Nc1ncnc2[nH]nc(-c3cc[nH]n3)c12]

In embodiments, the compound has the formula:

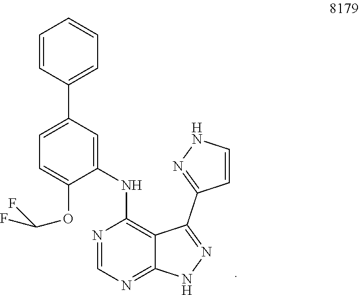 [SMILES: FC(F)Oc1ccc(-c2ccccc2)cc1Nc1ncnc2[nH]nc(-c3cc[nH]n3)c12]

In embodiments, the compound has the formula:

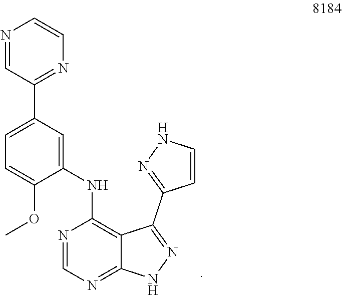 [SMILES: COc1ccc(-c2cnccn2)cc1Nc1ncnc2[nH]nc(-c3cc[nH]n3)c12]

In embodiments, the compound has the formula:

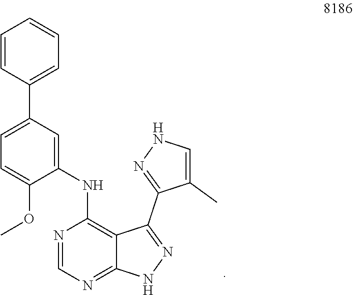 [SMILES: COc1ccc(-c2ccccc2)cc1Nc1ncnc2[nH]nc(-c3n[nH]cc3C)c12]

In embodiments, the compound has the formula:

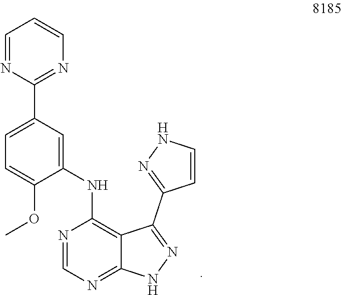 [SMILES: COc1ccc(-c2ncccn2)cc1Nc1ncnc2[nH]nc(-c3cc[nH]n3)c12]

In embodiments, the compound has the formula:

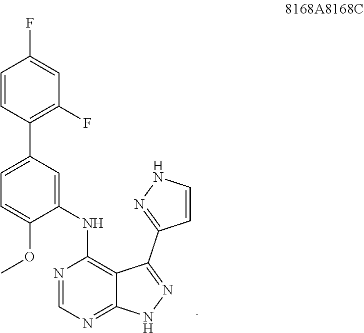 [SMILES: COc1ccc(-c2ccc(F)cc2F)cc1Nc1ncnc2[nH]nc(-c3cc[nH]n3)c12]

In embodiments, the compound has the formula:

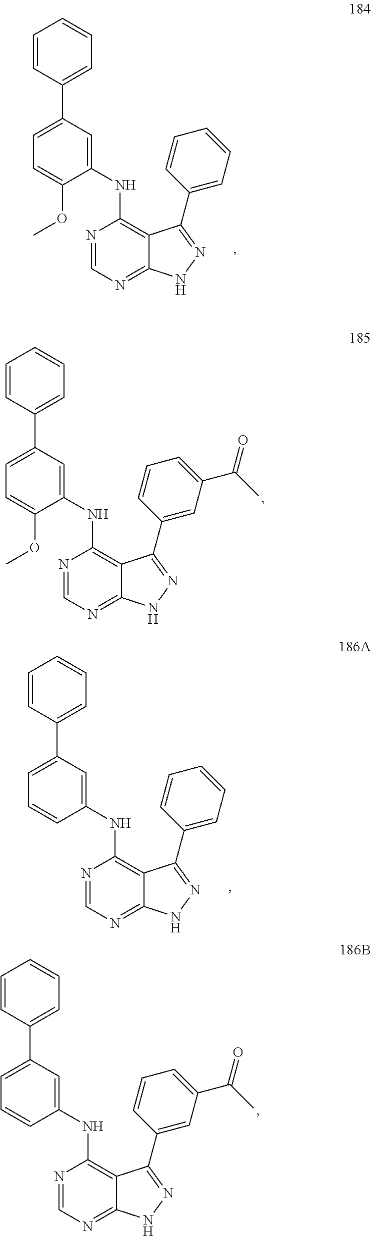 [SMILES: COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12 ; c1ccc(-c2cccc(Nc3ncnc4[nH]nc(-c5ccccc5)c34)c2)cc1 ; CC(=O)c1cccc(-c2n[nH]c3ncnc(Nc4cccc(-c5ccccc5)c4)c23)c1]

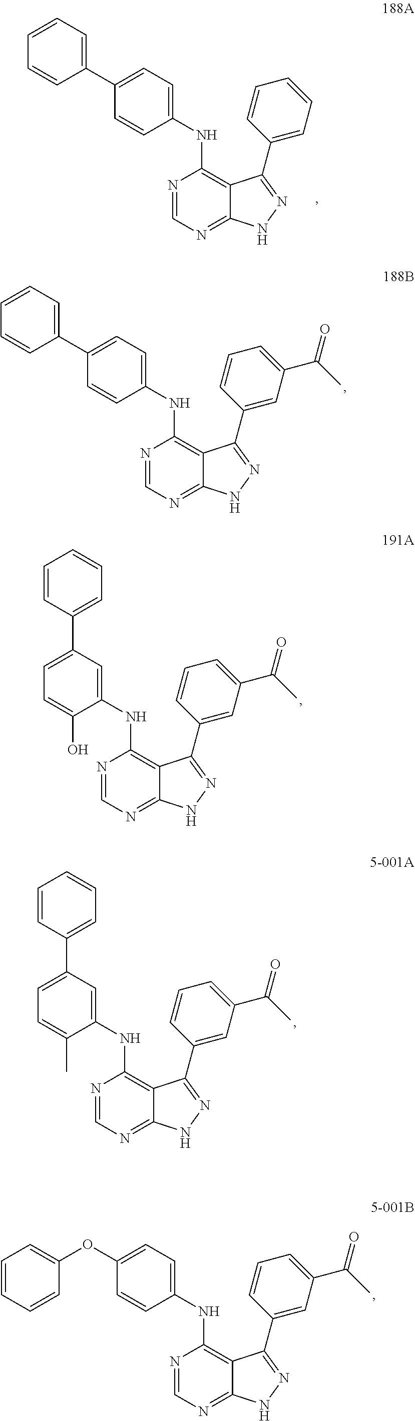 [SMILES: c1ccc(-c2ccc(Nc3ncnc4[nH]nc(-c5ccccc5)c34)cc2)cc1 ; CC(=O)c1cccc(-c2n[nH]c3ncnc(Nc4ccc(-c5ccccc5)cc4)c23)c1 ; CC(=O)c1cccc(-c2n[nH]c3ncnc(Nc4cc(-c5ccccc5)ccc4O)c23)c1 ; CC(=O)c1cccc(-c2n[nH]c3ncnc(Nc4cc(-c5ccccc5)ccc4C)c23)c1 ; CC(=O)c1cccc(-c2n[nH]c3ncnc(Nc4ccc(Oc5ccccc5)cc4)c23)c1]

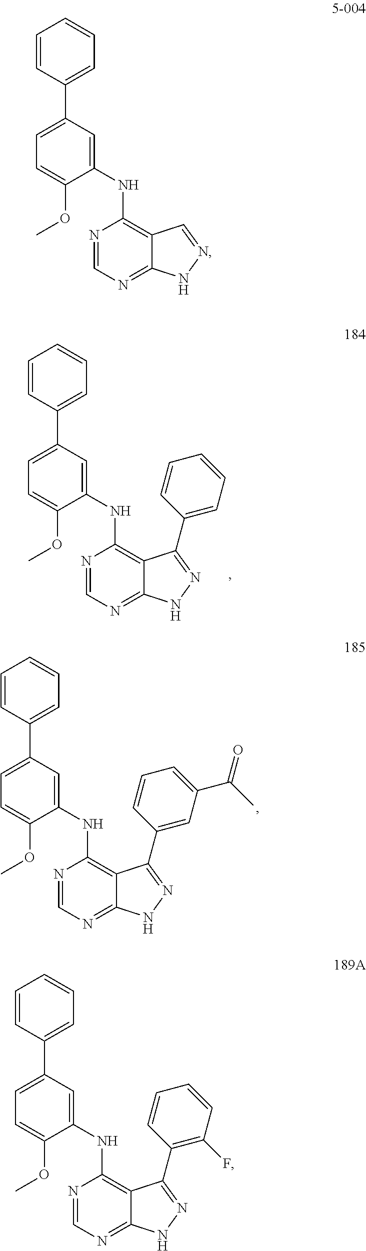 [SMILES: COc1ccc(-c2ccccc2)cc1Nc1ncnc2[nH]ncc12 ; COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12 ; COc1ccc(-c2ccccc2)cc1Nc1ncnc2[nH]nc(-c3ccccc3F)c12]

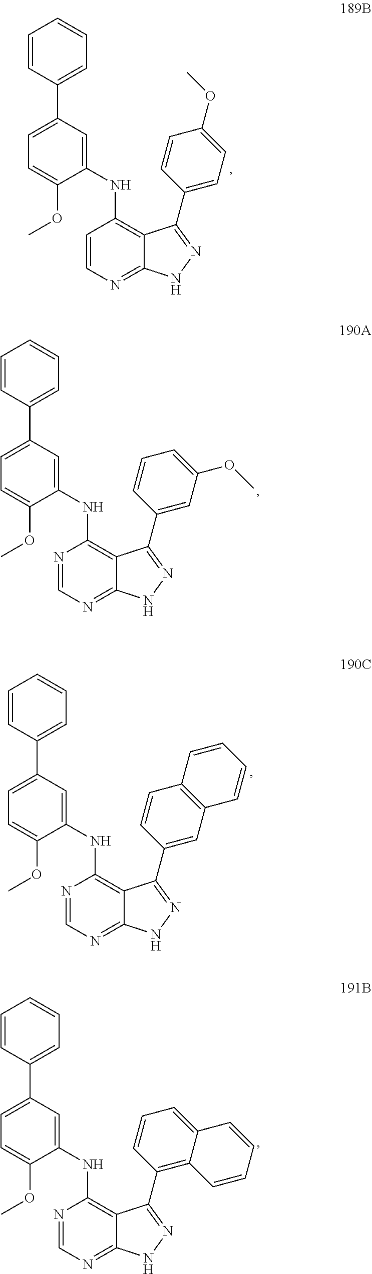 [SMILES: COc1ccc(-c2n[nH]c3nccc(Nc4cc(-c5ccccc5)ccc4OC)c23)cc1 ; COc1cccc(-c2n[nH]c3ncnc(Nc4cc(-c5ccccc5)ccc4OC)c23)c1 ; COc1ccc(-c2ccccc2)cc1Nc1ncnc2[nH]nc(-c3ccc4ccccc4c3)c12 ; COc1ccc(-c2ccccc2)cc1Nc1ncnc2[nH]nc(-c3cccc4ccccc34)c12]

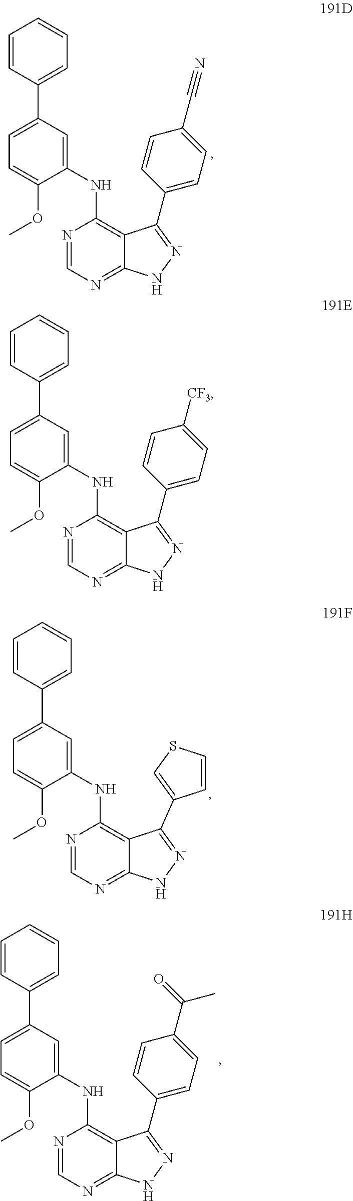 [SMILES: COc1ccc(-c2ccccc2)cc1Nc1ncnc2[nH]nc(-c3ccc(C#N)cc3)c12 ; COc1ccc(-c2ccccc2)cc1Nc1ncnc2[nH]nc(-c3ccc(C(F)(F)F)cc3)c12 ; COc1ccc(-c2ccccc2)cc1Nc1ncnc2[nH]nc(-c3ccsc3)c12 ; COc1ccc(-c2ccccc2)cc1Nc1ncnc2[nH]nc(-c3ccc(C(C)=O)cc3)c12]

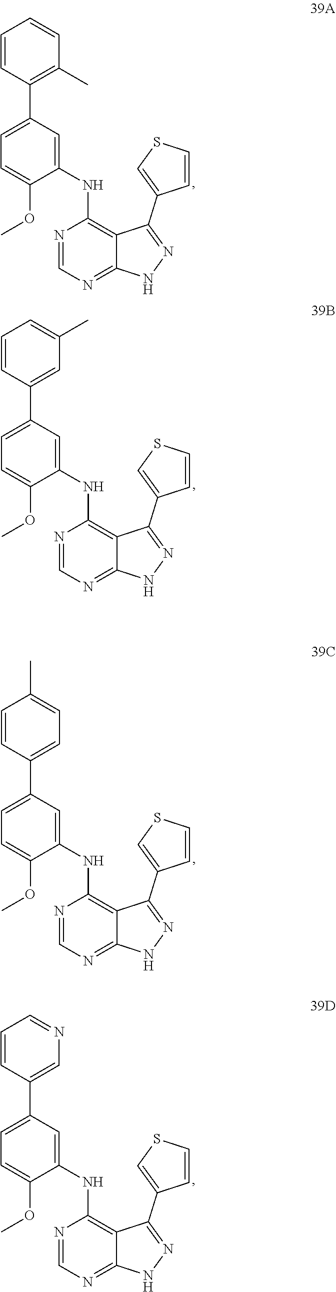

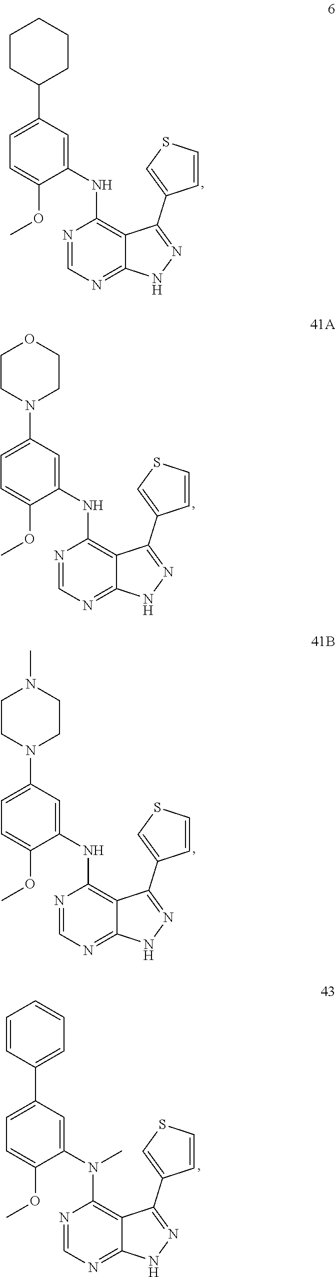 [SMILES: COc1ccc(C2CCCCC2)cc1Nc1ncnc2[nH]nc(-c3ccsc3)c12 ; COc1ccc(N2CCOCC2)cc1Nc1ncnc2[nH]nc(-c3ccsc3)c12 ; COc1ccc(N2CCN(C)CC2)cc1Nc1ncnc2[nH]nc(-c3ccsc3)c12 ; COc1ccc(-c2ccccc2)cc1N(C)c1ncnc2[nH]nc(-c3ccsc3)c12]

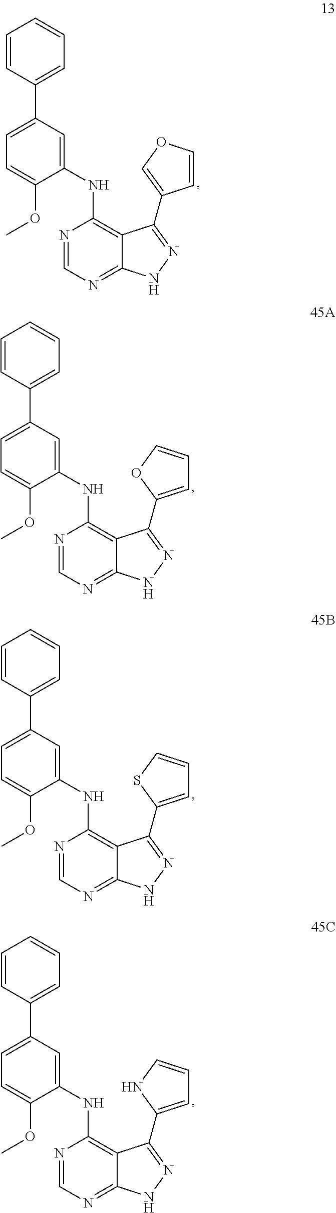 [SMILES: COc1ccc(-c2ccccc2)cc1Nc1ncnc2[nH]nc(-c3ccoc3)c12 ; COc1ccc(-c2ccccc2)cc1Nc1ncnc2[nH]nc(-c3ccco3)c12 ; COc1ccc(-c2ccccc2)cc1Nc1ncnc2[nH]nc(-c3cccs3)c12 ; COc1ccc(-c2ccccc2)cc1Nc1ncnc2[nH]nc(-c3ccc[nH]3)c12]

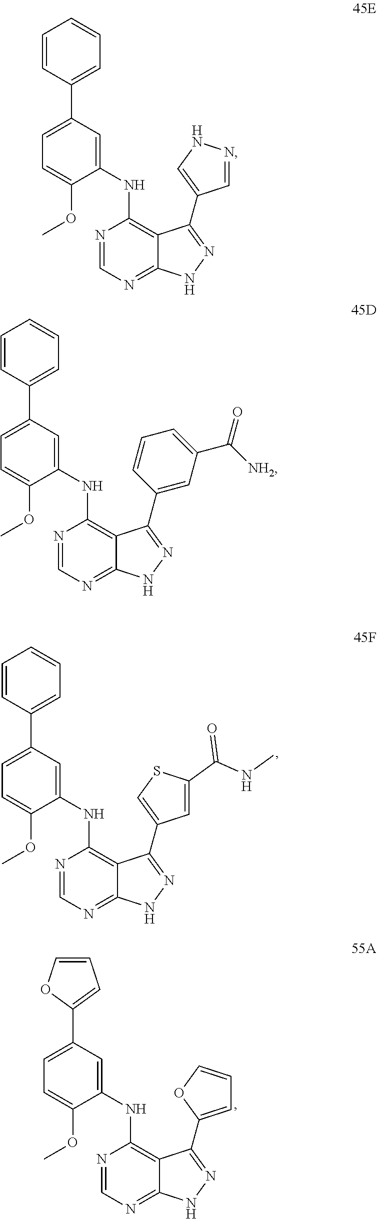

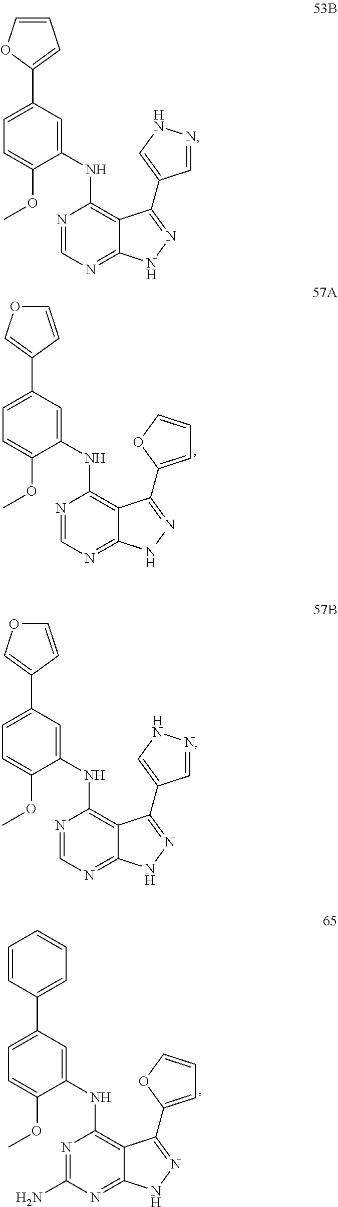 [SMILES: COc1ccc(-c2ccco2)cc1Nc1ncnc2[nH]nc(-c3cn[nH]c3)c12 ; COc1ccc(-c2ccoc2)cc1Nc1ncnc2[nH]nc(-c3ccco3)c12 ; COc1ccc(-c2ccoc2)cc1Nc1ncnc2[nH]nc(-c3cn[nH]c3)c12 ; COc1ccc(-c2ccccc2)cc1Nc1nc(N)nc2[nH]nc(-c3ccco3)c12]

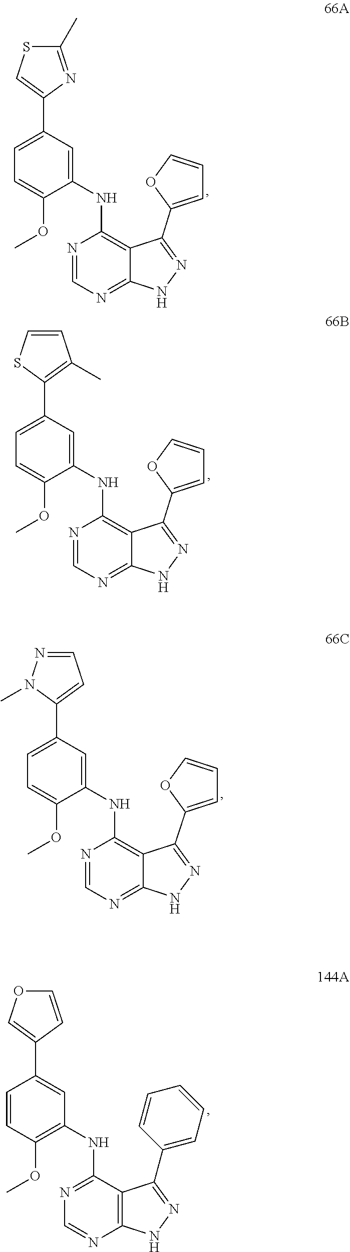 [SMILES: COc1ccc(-c2csc(C)n2)cc1Nc1ncnc2[nH]nc(-c3ccco3)c12 ; COc1ccc(-c2sccc2C)cc1Nc1ncnc2[nH]nc(-c3ccco3)c12 ; COc1ccc(-c2ccnn2C)cc1Nc1ncnc2[nH]nc(-c3ccco3)c12 ; COc1ccc(-c2ccoc2)cc1Nc1ncnc2[nH]nc(-c3ccccc3)c12]

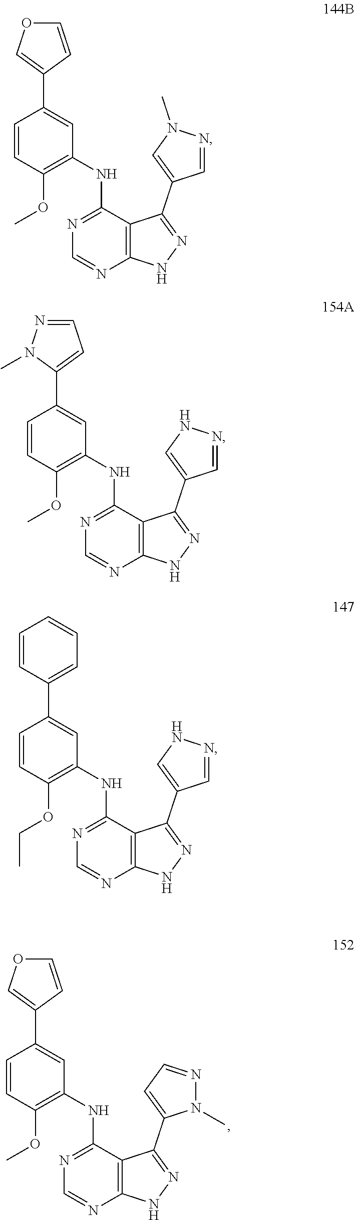 [SMILES: COc1ccc(-c2ccoc2)cc1Nc1ncnc2[nH]nc(-c3cnn(C)c3)c12 ; COc1ccc(-c2ccnn2C)cc1Nc1ncnc2[nH]nc(-c3cn[nH]c3)c12 ; CCOc1ccc(-c2ccccc2)cc1Nc1ncnc2[nH]nc(-c3cn[nH]c3)c12 ; COc1ccc(-c2ccoc2)cc1Nc1ncnc2[nH]nc(-c3ccnn3C)c12]

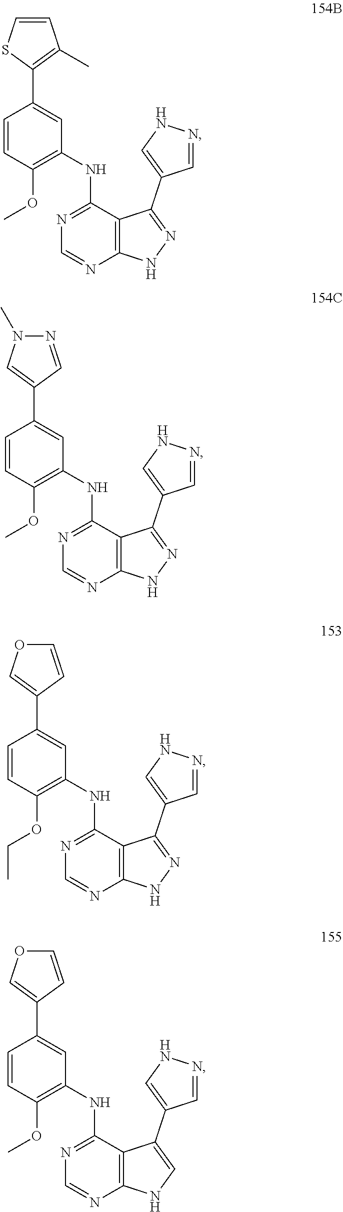 [SMILES: COc1ccc(-c2sccc2C)cc1Nc1ncnc2[nH]nc(-c3cn[nH]c3)c12 ; COc1ccc(-c2cnn(C)c2)cc1Nc1ncnc2[nH]nc(-c3cn[nH]c3)c12 ; CCOc1ccc(-c2ccoc2)cc1Nc1ncnc2[nH]nc(-c3cn[nH]c3)c12 ; COc1ccc(-c2ccoc2)cc1Nc1ncnc2[nH]cc(-c3cn[nH]c3)c12]

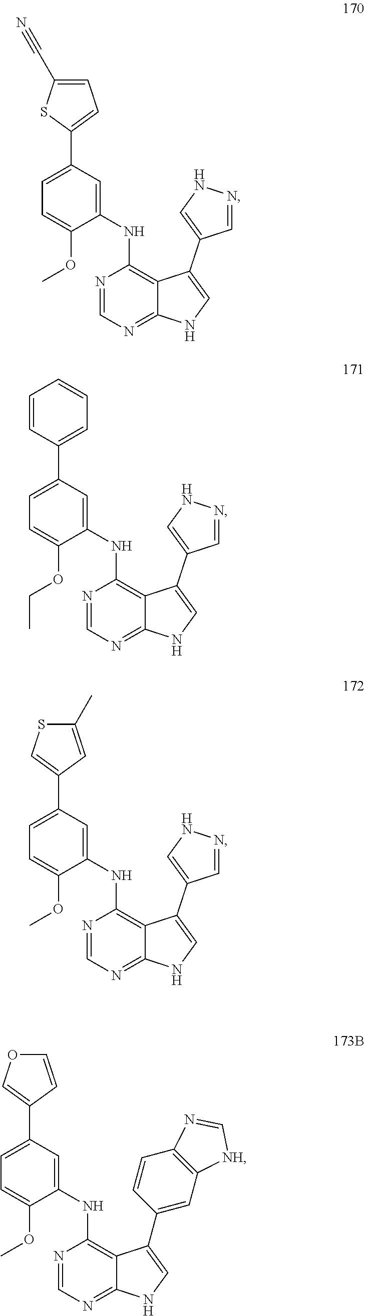 [SMILES: COc1ccc(-c2ccc(C#N)s2)cc1Nc1ncnc2[nH]cc(-c3cn[nH]c3)c12 ; CCOc1ccc(-c2ccccc2)cc1Nc1ncnc2[nH]cc(-c3cn[nH]c3)c12 ; COc1ccc(-c2csc(C)c2)cc1Nc1ncnc2[nH]cc(-c3cn[nH]c3)c12 ; COc1ccc(-c2ccoc2)cc1Nc1ncnc2[nH]cc(-c3ccc4nc[nH]c4c3)c12]

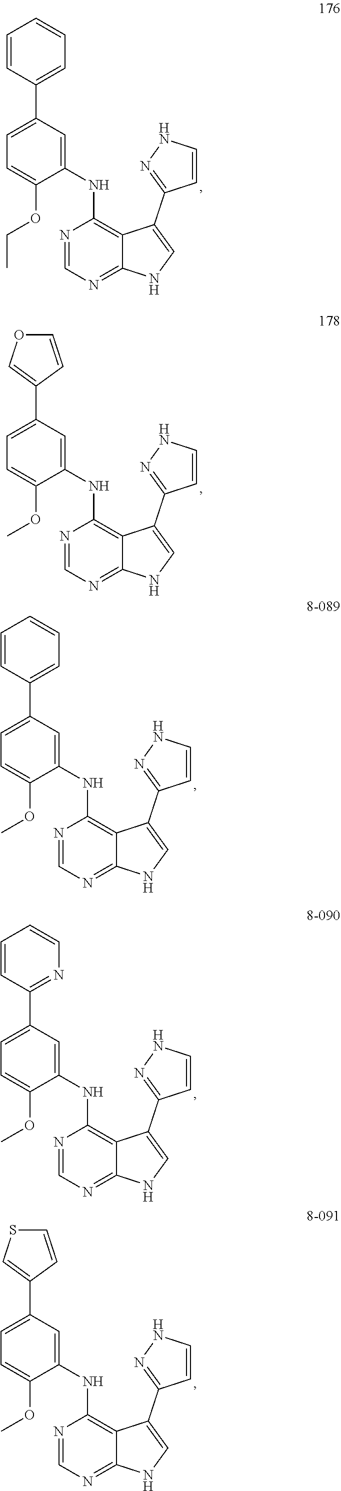 [SMILES: CCOc1ccc(-c2ccccc2)cc1Nc1ncnc2[nH]cc(-c3cc[nH]n3)c12 ; COc1ccc(-c2ccoc2)cc1Nc1ncnc2[nH]cc(-c3cc[nH]n3)c12 ; COc1ccc(-c2ccccc2)cc1Nc1ncnc2[nH]cc(-c3cc[nH]n3)c12 ; COc1ccc(-c2ccccn2)cc1Nc1ncnc2[nH]cc(-c3cc[nH]n3)c12 ; COc1ccc(-c2ccsc2)cc1Nc1ncnc2[nH]cc(-c3cc[nH]n3)c12]

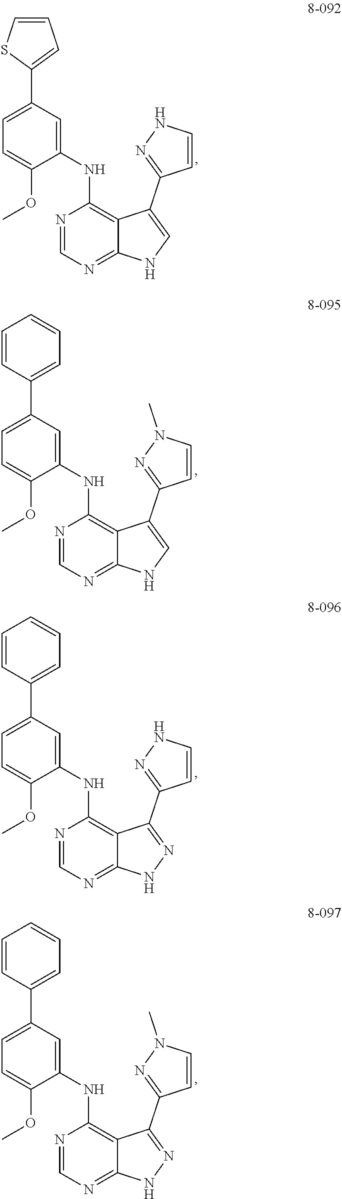 [SMILES: COc1ccc(-c2cccs2)cc1Nc1ncnc2[nH]cc(-c3cc[nH]n3)c12 ; COc1ccc(-c2ccccc2)cc1Nc1ncnc2[nH]cc(-c3ccn(C)n3)c12 ; COc1ccc(-c2ccccc2)cc1Nc1ncnc2[nH]nc(-c3cc[nH]n3)c12 ; COc1ccc(-c2ccccc2)cc1Nc1ncnc2[nH]nc(-c3ccn(C)n3)c12]

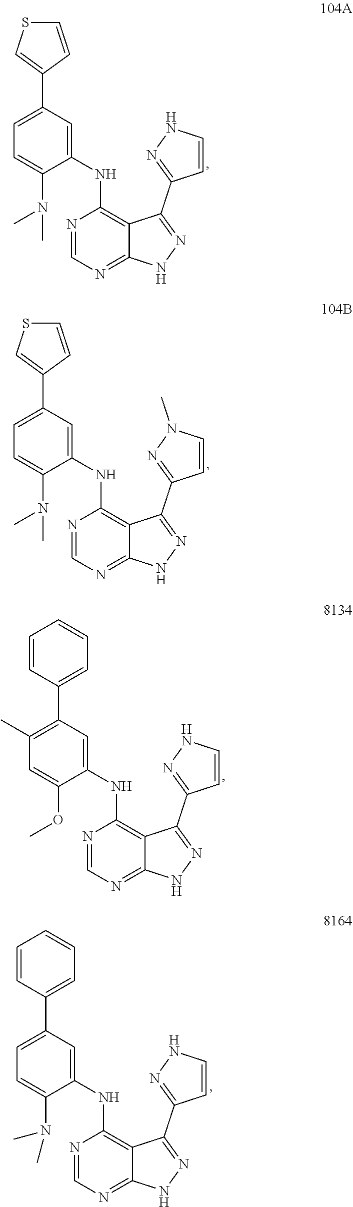 [SMILES: CN(C)c1ccc(-c2ccsc2)cc1Nc1ncnc2[nH]nc(-c3cc[nH]n3)c12 ; CN(C)c1ccc(-c2ccsc2)cc1Nc1ncnc2[nH]nc(-c3ccn(C)n3)c12 ; COc1cc(C)c(-c2ccccc2)cc1Nc1ncnc2[nH]nc(-c3cc[nH]n3)c12 ; CN(C)c1ccc(-c2ccccc2)cc1Nc1ncnc2[nH]nc(-c3cc[nH]n3)c12]

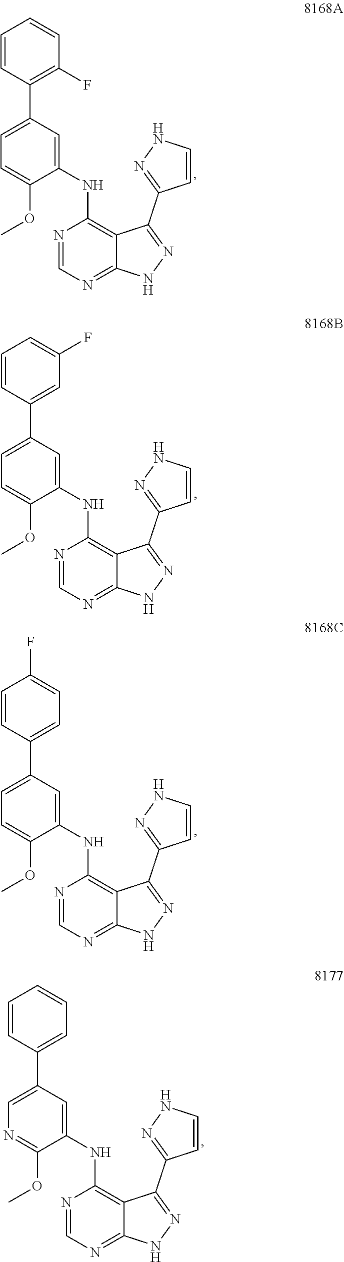 [SMILES: COc1ccc(-c2ccccc2F)cc1Nc1ncnc2[nH]nc(-c3cc[nH]n3)c12 ; COc1ccc(-c2cccc(F)c2)cc1Nc1ncnc2[nH]nc(-c3cc[nH]n3)c12 ; COc1ccc(-c2ccc(F)cc2)cc1Nc1ncnc2[nH]nc(-c3cc[nH]n3)c12 ; COc1ncc(-c2ccccc2)cc1Nc1ncnc2[nH]nc(-c3cc[nH]n3)c12]

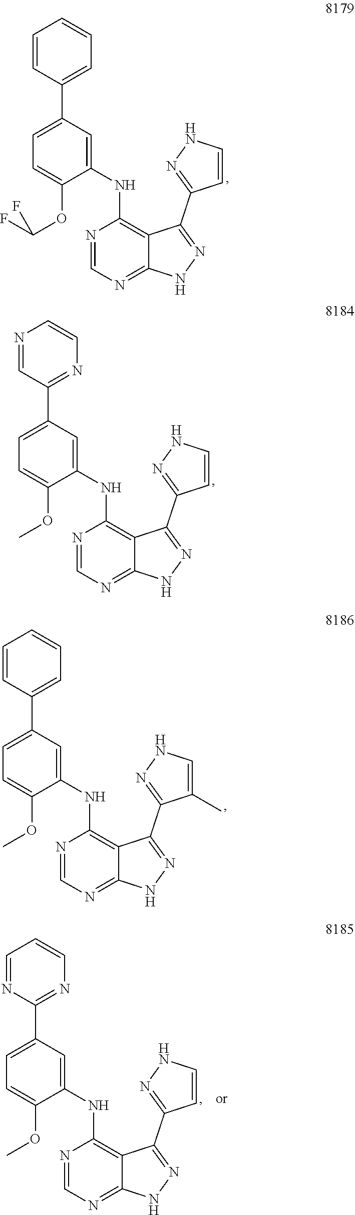 [SMILES: FC(F)Oc1ccc(-c2ccccc2)cc1Nc1ncnc2[nH]nc(-c3cc[nH]n3)c12 ; COc1ccc(-c2cnccn2)cc1Nc1ncnc2[nH]nc(-c3cc[nH]n3)c12 ; COc1ccc(-c2ccccc2)cc1Nc1ncnc2[nH]nc(-c3n[nH]cc3C)c12 ; COc1ccc(-c2ncccn2)cc1Nc1ncnc2[nH]nc(-c3cc[nH]n3)c12]

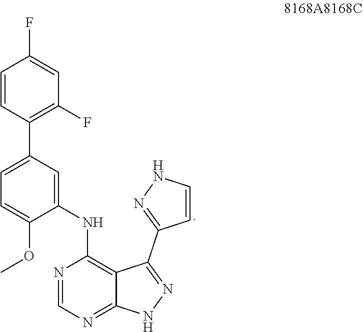 [SMILES: COc1ccc(-c2ccc(F)cc2F)cc1Nc1ncnc2[nH]nc(-c3cc[nH]n3)c12]

In embodiments, the compound has the formula:

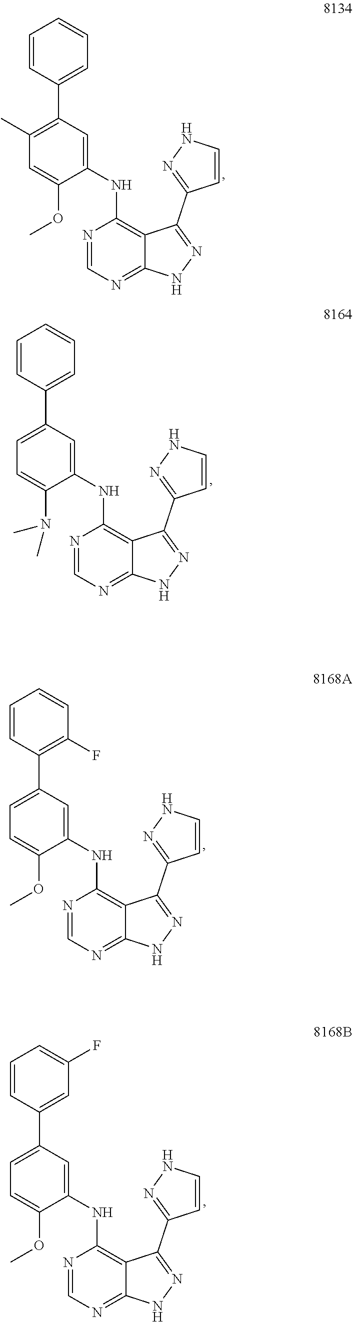 [SMILES: COc1cc(C)c(-c2ccccc2)cc1Nc1ncnc2[nH]nc(-c3cc[nH]n3)c12 ; CN(C)c1ccc(-c2ccccc2)cc1Nc1ncnc2[nH]nc(-c3cc[nH]n3)c12 ; COc1ccc(-c2ccccc2F)cc1Nc1ncnc2[nH]nc(-c3cc[nH]n3)c12 ; COc1ccc(-c2cccc(F)c2)cc1Nc1ncnc2[nH]nc(-c3cc[nH]n3)c12]

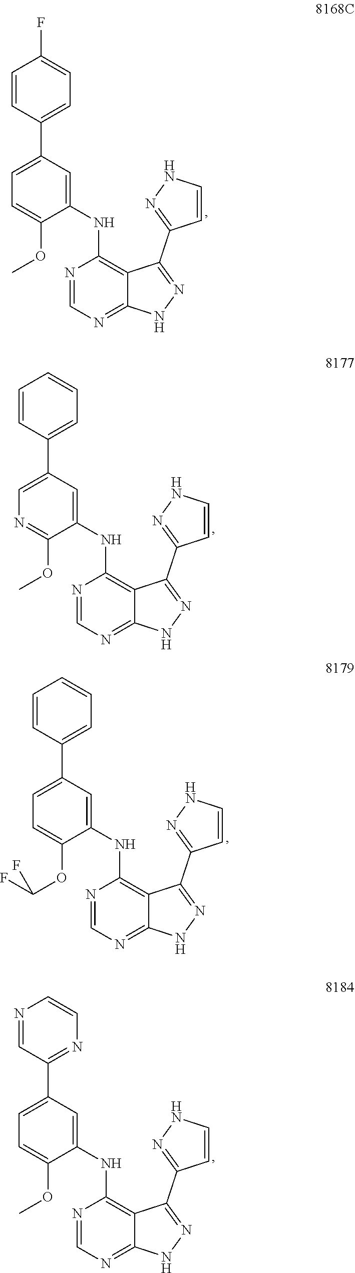 [SMILES: COc1ccc(-c2ccc(F)cc2)cc1Nc1ncnc2[nH]nc(-c3cc[nH]n3)c12 ; COc1ncc(-c2ccccc2)cc1Nc1ncnc2[nH]nc(-c3cc[nH]n3)c12 ; FC(F)Oc1ccc(-c2ccccc2)cc1Nc1ncnc2[nH]nc(-c3cc[nH]n3)c12 ; COc1ccc(-c2cnccn2)cc1Nc1ncnc2[nH]nc(-c3cc[nH]n3)c12]

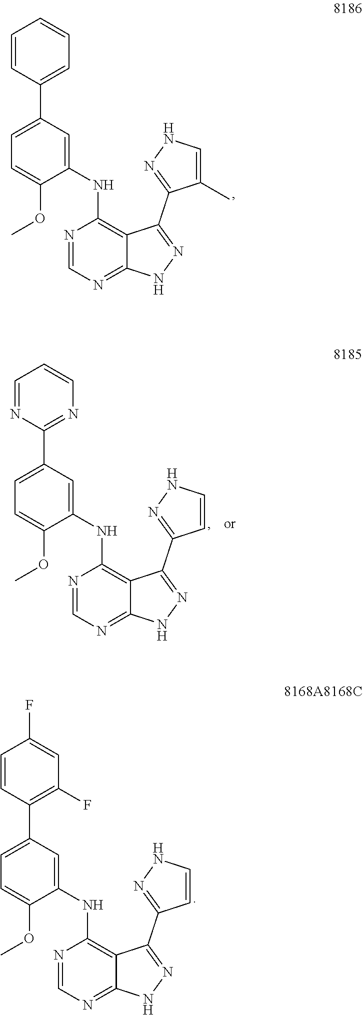 [SMILES: COc1ccc(-c2ccccc2)cc1Nc1ncnc2[nH]nc(-c3n[nH]cc3C)c12 ; COc1ccc(-c2ncccn2)cc1Nc1ncnc2[nH]nc(-c3cc[nH]n3)c12 ; COc1ccc(-c2ccc(F)cc2F)cc1Nc1ncnc2[nH]nc(-c3cc[nH]n3)c12]

In embodiments, the compound has the formula:

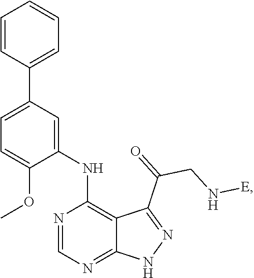

wherein E is as described herein. In embodiments, the compound has the formula:

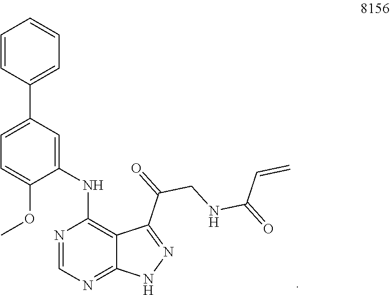 [SMILES: C=CC(=O)NCC(=O)c1n[nH]c2ncnc(Nc3cc(-c4ccccc4)ccc3OC)c12]

In embodiments, a compound is a compound described herein, including in an aspect, embodiment, table, figure, example, scheme, or claim.

In embodiments, the compound is not

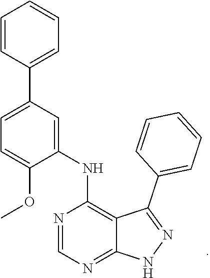 [SMILES: COc1ccc(-c2ccccc2)cc1Nc1ncnc2[nH]nc(-c3ccccc3)c12]

In embodiments, the compound is not

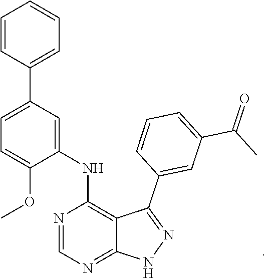 [SMILES: COc1ccc(-c2ccccc2)cc1Nc1ncnc2[nH]nc(-c3cccc(C(C)=O)c3)c12]

In embodiments, the compound is not

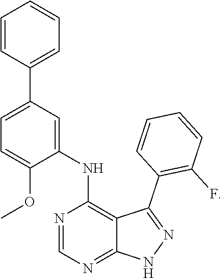 [SMILES: COc1ccc(-c2ccccc2)cc1Nc1ncnc2[nH]nc(-c3ccccc3F)c12]

In embodiments, the compound is not

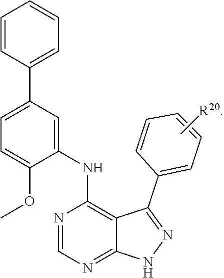

In embodiments, the compound is not

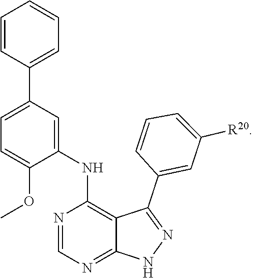

In embodiments, the compound is not

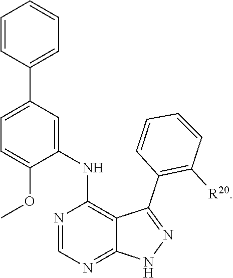

In embodiments, the compound is not

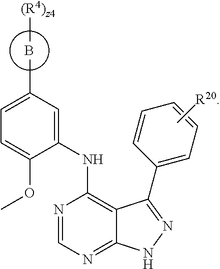

In embodiments, the compound is not

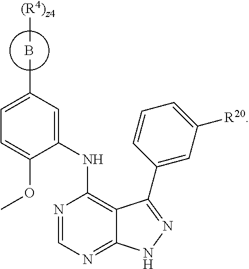

In embodiments, the compound is not

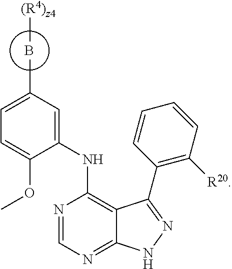

In embodiments, the compound is not

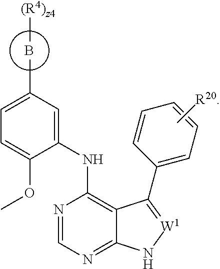

In embodiments, the compound is not

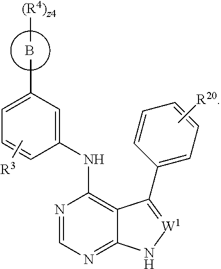

In embodiments, the compound is not a compound described in WO 2011/149827. In embodiments, the compound is not a compound described in Lawhorn et al. (J. Med Chem. 2915, 58, 7431-7448). In embodiments, the compound is not a compound described in an example, table, or figure herein. In embodiments, the compound is not compound 184. In embodiments, the compound is not compound 185. In embodiments, the compound is not compound 187A. In embodiments, the compound is not compound 187B. In embodiments, the compound is not compound 186A. In embodiments, the compound is not compound 186B. In embodiments, the compound is not compound 188A. In embodiments, the compound is not compound 188B. In embodiments, the compound is not compound 190D. In embodiments, the compound is not compound 191A. In embodiments, the compound is not compound 5-001A. In embodiments, the compound is not compound 5-001B. In embodiments, the compound is not compound 5-004. In embodiments, the compound is not compound 184. In embodiments, the compound is not compound 185. In embodiments, the compound is not compound 189A. In embodiments, the compound is not compound 189B. In embodiments, the compound is not compound 190A. In embodiments, the compound is not compound 190C. In embodiments, the compound is not compound191B. In embodiments, the compound is not compound 191D. In embodiments, the compound is not compound 191E. In embodiments, the compound is not compound 191F. In embodiments, the compound is not compound 191H. In embodiments, the compound is not compound 39A. In embodiments, the compound is not compound 39B. In embodiments, the compound is not compound 39C. In embodiments, the compound is not compound 39D. In embodiments, the compound is not compound 6. In embodiments, the compound is not compound 41A. In embodiments, the compound is not compound 41B. In embodiments, the compound is not compound 42. In embodiments, the compound is not compound 43. In embodiments, the compound is not compound 13. In embodiments, the compound is not compound 45A. In embodiments, the compound is not compound 45B. In embodiments, the compound is not compound 45C. In embodiments, the compound is not compound 45E. In embodiments, the compound is not compound 45D. In embodiments, the compound is not compound 45F. In embodiments, the compound is not compound 55A. In embodiments, the compound is not compound 53B. In embodiments, the compound is not compound 57A. In embodiments, the compound is not compound 57B. In embodiments, the compound is not compound 45A. In embodiments, the compound is not compound 45E. In embodiments, the compound is not compound 53B. In embodiments, the compound is not compound 55A. In embodiments, the compound is not compound 57A. In embodiments, the compound is not compound 57B. In embodiments, the compound is not compound 65. In embodiments, the compound is not compound 66A. In embodiments, the compound is not compound 66B. In embodiments, the compound is not compound 66C. In embodiments, the compound is not compound 57B. In embodiments, the compound is not compound 144A. In embodiments, the compound is not compound 144B. In embodiments, the compound is not compound 154A. In embodiments, the compound is not compound 45E. In embodiments, the compound is not compound 147. In embodiments, the compound is not compound 152. In embodiments, the compound is not compound 57A. In embodiments, the compound is not compound 154B. In embodiments, the compound is not compound 154C. In embodiments, the compound is not compound 153. In embodiments, the compound is not compound 155. In embodiments, the compound is not compound 170. In embodiments, the compound is not compound 171. In embodiments, the compound is not compound 172. In embodiments, the compound is not compound 173B. In embodiments, the compound is not compound 176. In embodiments, the compound is not compound 178. In embodiments, the compound is not compound 8-0089. In embodiments, the compound is not compound 8-090. In embodiments, the compound is not compound 8-091. In embodiments, the compound is not compound 8-092. In embodiments, the compound is not compound 8-095. In embodiments, the compound is not compound 8-096. In embodiments, the compound is not compound 8-097. In embodiments, the compound is not compound 8-091. In embodiments, the compound is not compound 104A. In embodiments, the compound is not compound 104B.

In embodiments, W¹ is not N. In embodiments, W¹ is not CH.

In embodiments, R¹ is not hydrogen. In embodiments, R¹ is not substituted or unsubstituted phenyl or substituted or unsubstituted 5 to 6 membered heteroaryl. In embodiments, R¹ is not substituted or unsubstituted phenyl. In embodiments, R¹ is not unsubstituted phenyl. In embodiments, R¹ is not substituted or unsubstituted 5 to 6 membered heteroaryl. In embodiments, R¹ is not unsubstituted 5 to 6 membered heteroaryl. In embodiments, R¹ is not substituted phenyl. In embodiments, R¹ is not substituted pyridyl. In embodiments, R¹ is not substituted cyclohexyl. In embodiments, R¹ is not substituted morpholinyl. In embodiments, R¹ is not substituted piperazinyl. In embodiments, R¹ is not substituted furanyl. In embodiments, R¹ is not substituted thiazolyl. In embodiments, R¹ is not substituted pyrazolyl. In embodiments, R¹ is not substituted thienyl. In embodiments, R¹ is not substituted pyrazinyl. In embodiments, R¹ is not substituted pyrimidinyl. In embodiments, R¹ is not substituted pyridazinyl. In embodiments, R¹ is not substituted triazinyl. In embodiments, R¹ is not substituted tetrazinyl. In embodiments, R¹ is not substituted tetrazolyl. In embodiments, R¹ is not substituted triazolyl. In embodiments, R¹ is not substituted quinolinyl. In embodiments, R¹ is not substituted isoquinolinyl. In embodiments, R¹ is not substituted quinazolinyl. In embodiments, R¹ is not substituted quinoxalinyl. In embodiments, R¹ is not substituted imidazolyl. In embodiments, R¹ is not substituted oxazolyl. In embodiments, R¹ is not substituted isoxazolyl. In embodiments, R¹ is not substituted thiazolyl. In embodiments, R¹ is not substituted piperidinyl. In embodiments, R¹ is not substituted thiomorpholinyl. In embodiments, R¹ is not substituted thianyl. In embodiments, R¹ is not substituted oxanyl. In embodiments, R¹ is not substituted tetrahydropuranyl. In embodiments, R¹ is not substituted dihydropuranyl. In embodiments, R¹ is not substituted dioxanyl. In embodiments, R¹ is not substituted pyrazolyl. In embodiments, R¹ is not substituted pyrrolyl. In embodiments, R¹ is not substituted thienyl. In embodiments, R¹ is not substituted benzofuranyl. In embodiments, R¹ is not substituted indolyl. In embodiments, R¹ is not substituted benzothienyl. In embodiments, R¹ is not substituted benzimidazolyl. In embodiments, R¹ is not substituted isobenzofuranyl. In embodiments, R¹ is not substituted isoindolyl. In embodiments, R¹ is not substituted benzo[c]thienyl. In embodiments, R¹ is not substituted purinyl. In embodiments, R¹ is not substituted indazolyl. In embodiments, R¹ is not substituted benzoxazolyl. In embodiments, R¹ is not substituted benzisoxazolyl. In embodiments, R¹ is not substituted benzothiazolyl. In embodiments, R¹ is not substituted cyclopentyl. In embodiments, R¹ is not substituted cyclobutyl. In embodiments, R¹ is not substituted naphthyl. In embodiments, R¹ is not substituted 1-naphthyl. In embodiments, R¹ is not substituted 2-naphthyl. In embodiments, R¹ is not hydrogen. In embodiments, R¹ is not substituted 2-thienyl. In embodiments, R¹ is not substituted 3-thienyl. In embodiments, R¹ is not substituted 2-furanyl. In embodiments, R¹ is not substituted 3-furanyl. In embodiments, R¹ is not substituted 2-pyridyl. In embodiments, R¹ is not substituted 3-pyridyl. In embodiments, R¹ is not substituted 4-pyridyl. In embodiments, R¹ is not substituted 3-pyrazolyl. In embodiments, R¹ is not substituted 4-pyrazolyl. In embodiments, R¹ is not substituted 5-pyrazolyl. In embodiments, R¹ is not substituted 2-pyrrolyl. In embodiments, R¹ is not substituted 3-pyrrolyl.

In embodiments, R¹ is not R²⁰-substituted phenyl. In embodiments, R¹ is not R²⁰-substituted pyridyl. In embodiments, R¹ is not R²⁰-substituted cyclohexyl. In embodiments, R¹ is not R²⁰-substituted morpholinyl. In embodiments, R¹ is not R²⁰-substituted piperazinyl. In embodiments, R¹ is not R²⁰-substituted furanyl. In embodiments, R¹ is not R²⁰-substituted thiazolyl. In embodiments, R¹ is not R²⁰-substituted pyrazolyl. In embodiments, R¹ is not R²⁰-substituted thienyl. In embodiments, R¹ is not R²⁰-substituted pyrazinyl. In embodiments, R¹ is not R²⁰-substituted pyrimidinyl. In embodiments, R¹ is not R²⁰-substituted pyridazinyl. In embodiments, R¹ is not R²⁰-substituted triazinyl. In embodiments, R¹ is not R²⁰-substituted tetrazinyl. In embodiments, R¹ is not R²⁰-substituted tetrazolyl. In embodiments, R¹ is not R²⁰-substituted triazolyl. In embodiments, R¹ is not R²⁰-substituted quinolinyl. In embodiments, R¹ is not R²⁰-substituted isoquinolinyl. In embodiments, R¹ is not R²⁰-substituted quinazolinyl. In embodiments, R¹ is not R²⁰-substituted quinoxalinyl. In embodiments, R¹ is not R²⁰-substituted imidazolyl. In embodiments, R¹ is not R²⁰-substituted oxazolyl. In embodiments, R¹ is not R²⁰-substituted isoxazolyl. In embodiments, R¹ is not R²⁰-substituted thiazolyl. In embodiments, R¹ is not R²⁰-substituted piperidinyl. In embodiments, R¹ is not R²⁰-substituted thiomorpholinyl. In embodiments, R¹ is not R²⁰-substituted thianyl. In embodiments, R¹ is not R²⁰-substituted oxanyl. In embodiments, R¹ is not R²⁰-substituted tetrahydropuranyl. In embodiments, R¹ is not R²⁰-substituted dihydropuranyl. In embodiments, R¹ is not R²⁰-substituted dioxanyl. In embodiments, R¹ is not R²⁰-substituted pyrazolyl. In embodiments, R¹ is not R²⁰-substituted pyrrolyl. In embodiments, R¹ is not R²⁰-substituted thienyl. In embodiments, R¹ is not R²⁰-substituted benzofuranyl. In embodiments, R¹ is not R²⁰-substituted indolyl. In embodiments, R¹ is not R²⁰-substituted benzothienyl. In embodiments, R¹ is not R²⁰-substituted benzimidazolyl. In embodiments, R¹ is not R²⁰-substituted isobenzofuranyl. In embodiments, R¹ is not R²⁰-substituted isoindolyl. In embodiments, R¹ is not R²⁰-substituted benzo[c]thienyl. In embodiments, R¹ is not R²⁰-substituted purinyl. In embodiments, R¹ is not R²⁰-substituted indazolyl. In embodiments, R¹ is not R²⁰-substituted benzoxazolyl. In embodiments, R¹ is not R²⁰-substituted benzisoxazolyl. In embodiments, R¹ is not R²⁰-substituted benzothiazolyl. In embodiments, R¹ is not R²⁰-substituted cyclopentyl. In embodiments, R¹ is not R²⁰-substituted cyclobutyl. In embodiments, R¹ is not R²⁰-substituted naphthyl. In embodiments, R¹ is not R²⁰-substituted 1-naphthyl. In embodiments, R¹ is not R²⁰-substituted 2-naphthyl. In embodiments, R¹ is not R²⁰-substituted 2-thienyl. In embodiments, R¹ is not R²⁰-substituted 3-thienyl. In embodiments, R¹ is not R²⁰-substituted 2-furanyl. In embodiments, R¹ is not R²⁰-substituted 3-furanyl. In embodiments, R¹ is not R²⁰-substituted 2-pyridyl. In embodiments, R¹ is not R²⁰-substituted 3-pyridyl. In embodiments, R¹ is not R²⁰-substituted 4-pyridyl. In embodiments, R¹ is not R²⁰-substituted 3-pyrazolyl. In embodiments, R¹ is not R²⁰-substituted 4-pyrazolyl. In embodiments, R¹ is not R²⁰-substituted 5-pyrazolyl. In embodiments, R¹ is not R²⁰-substituted 2-pyrrolyl. In embodiments, R¹ is not R²⁰-substituted 3-pyrrolyl.

In embodiments, R¹ is not unsubstituted phenyl. In embodiments, R¹ is not unsubstituted pyridyl. In embodiments, R¹ is not unsubstituted cyclohexyl. In embodiments, R¹ is not unsubstituted morpholinyl. In embodiments, R¹ is not unsubstituted piperazinyl. In embodiments, R¹ is not unsubstituted furanyl. In embodiments, R¹ is not unsubstituted thiazolyl. In embodiments, R¹ is not unsubstituted pyrazolyl. In embodiments, R¹ is not unsubstituted thienyl. In embodiments, R¹ is not unsubstituted pyrazinyl. In embodiments, R¹ is not unsubstituted pyrimidinyl. In embodiments, R¹ is not unsubstituted pyridazinyl. In embodiments, R¹ is not unsubstituted triazinyl. In embodiments, R¹ is not unsubstituted tetrazinyl. In embodiments, R¹ is not unsubstituted tetrazolyl. In embodiments, R¹ is not unsubstituted triazolyl. In embodiments, R¹ is not unsubstituted quinolinyl. In embodiments, R¹ is not unsubstituted isoquinolinyl. In embodiments, R¹ is not unsubstituted quinazolinyl. In embodiments, R¹ is not unsubstituted quinoxalinyl. In embodiments, R¹ is not unsubstituted imidazolyl. In embodiments, R¹ is not unsubstituted oxazolyl. In embodiments, R¹ is not unsubstituted isoxazolyl. In embodiments, R¹ is not unsubstituted thiazolyl. In embodiments, R¹ is not unsubstituted piperidinyl. In embodiments, R¹ is not unsubstituted thiomorpholinyl. In embodiments, R¹ is not unsubstituted thianyl. In embodiments, R¹ is not unsubstituted oxanyl. In embodiments, R¹ is not unsubstituted tetrahydropuranyl. In embodiments, R¹ is not unsubstituted dihydropuranyl. In embodiments, R¹ is not unsubstituted dioxanyl. In embodiments, R¹ is not unsubstituted pyrazolyl. In embodiments, R¹ is not unsubstituted pyrrolyl. In embodiments, R¹ is not unsubstituted thienyl. In embodiments, R¹ is not unsubstituted benzofuranyl. In embodiments, R¹ is not unsubstituted indolyl. In embodiments, R¹ is not unsubstituted benzothienyl. In embodiments, R¹ is not unsubstituted benzimidazolyl. In embodiments, R¹ is not unsubstituted isobenzofuranyl. In embodiments, R¹ is not unsubstituted isoindolyl. In embodiments, R¹ is not unsubstituted benzo[c]thienyl. In embodiments, R¹ is not unsubstituted purinyl. In embodiments, R¹ is not unsubstituted indazolyl. In embodiments, R¹ is not unsubstituted benzoxazolyl. In embodiments, R¹ is not unsubstituted benzisoxazolyl. In embodiments, R¹ is not unsubstituted benzothiazolyl. In embodiments, R¹ is not unsubstituted cyclopentyl. In embodiments, R¹ is not unsubstituted cyclobutyl. In embodiments, R¹ is not unsubstituted naphthyl. In embodiments, R¹ is not unsubstituted 1-naphthyl. In embodiments, R¹ is not unsubstituted 2-naphthyl. In embodiments, R¹ is not unsubstituted 2-thienyl. In embodiments, R¹ is not unsubstituted 3-thienyl. In embodiments, R¹ is not unsubstituted 2-furanyl. In embodiments, R¹ is not unsubstituted 3-furanyl. In embodiments, R¹ is not unsubstituted 2-pyridyl. In embodiments, R¹ is not unsubstituted 3-pyridyl. In embodiments, R¹ is not unsubstituted 4-pyridyl. In embodiments, R¹ is not unsubstituted 3-pyrazolyl. In embodiments, R¹ is not unsubstituted 4-pyrazolyl. In embodiments, R¹ is not unsubstituted 5-pyrazolyl. In embodiments, R¹ is not unsubstituted 2-pyrrolyl. In embodiments, R¹ is not unsubstituted 3-pyrrolyl.

In embodiments, R¹ is not substituted aryl. In embodiments, R¹ is not unsubstituted aryl. In embodiments, R¹ is not substituted C₆-C₁₀ aryl. In embodiments, R¹ is not unsubstituted C₆-C₁₀ aryl. In embodiments, R¹ is not substituted phenyl. In embodiments, R¹ is not unsubstituted phenyl. In embodiments, R¹ is not substituted heteroaryl. In embodiments, R¹ is not unsubstituted heteroaryl. In embodiments, R¹ is not substituted 5 to 10 membered heteroaryl. In embodiments, R¹ is not substituted 5 to 9 membered heteroaryl. In embodiments, R¹ is not unsubstituted 5 to 10 membered heteroaryl. In embodiments, R¹ is not unsubstituted 5 to 9 membered heteroaryl. In embodiments, R¹ is not substituted 5 to 6 membered heteroaryl. In embodiments, R¹ is not unsubstituted 5 to 6 membered heteroaryl. In embodiments, R¹ is not substituted 9 membered heteroaryl. In embodiments, R¹ is not substituted 10 membered heteroaryl. In embodiments, R¹ is not unsubstituted 9 membered heteroaryl. In embodiments, R¹ is not unsubstituted 10 membered heteroaryl. In embodiments, R¹ is not substituted 5 membered heteroaryl. In embodiments, R¹ is not substituted 6 membered heteroaryl. In embodiments, R¹ is not unsubstituted 5 membered heteroaryl. In embodiments, R¹ is not unsubstituted 6 membered heteroaryl.

In embodiments, R¹ is not substituted or unsubstituted pyrazolyl. In embodiments, R¹ is not substituted or unsubstituted pyridyl. In embodiments, R¹ is not substituted or unsubstituted imidazolyl. In embodiments, R¹ is not substituted or unsubstituted oxazolyl. In embodiments, R¹ is not substituted or unsubstituted isoxazolyl. In embodiments, R¹ is not substituted or unsubstituted thiazolyl. In embodiments, R¹ is not substituted or unsubstituted furanyl. In embodiments, R¹ is not substituted or unsubstituted pyrrolyl. In embodiments, R¹ is not substituted or unsubstituted thienyl. In embodiments, R¹ is not substituted pyrazolyl. In embodiments, R¹ is not substituted pyridyl. In embodiments, R¹ is not substituted imidazolyl. In embodiments, R¹ is not substituted oxazolyl. In embodiments, R¹ is not substituted isoxazolyl. In embodiments, R¹ is not substituted thiazolyl. In embodiments, R¹ is not substituted furanyl. In embodiments, R¹ is not substituted pyrrolyl. In embodiments, R¹ is not substituted thienyl.

In embodiments, R¹ is not unsubstituted pyrazolyl. In embodiments, R¹ is not unsubstituted pyridyl. In embodiments, R¹ is not unsubstituted imidazolyl. In embodiments, R¹ is not unsubstituted oxazolyl. In embodiments, R¹ is not unsubstituted isoxazolyl. In embodiments, R¹ is not unsubstituted thiazolyl. In embodiments, R¹ is not unsubstituted furanyl. In embodiments, R¹ is not unsubstituted pyrrolyl. In embodiments, R¹ is not unsubstituted thienyl.

In embodiments, R¹ is not methyl-substituted pyrazolyl. In embodiments, R¹ is not methyl-substituted pyridyl. In embodiments, R¹ is not methyl-substituted imidazolyl. In embodiments, R¹ is not methyl-substituted oxazolyl. In embodiments, R¹ is not methyl-substituted isoxazolyl. In embodiments, R¹ is not methyl-substituted thiazolyl. In embodiments, R¹ is not methyl-substituted furanyl. In embodiments, R¹ is not methyl-substituted pyrrolyl. In embodiments, R¹ is not methyl-substituted thienyl.

In embodiments, R¹ is not independently R²⁰-substituted or unsubstituted aryl or R²⁰-substituted or unsubstituted heteroaryl. In embodiments, R¹ is not independently R²⁰-substituted or unsubstituted phenyl or R²⁰-substituted or unsubstituted 5 to 6 membered heteroaryl. In embodiments, X¹ is not —F, —Cl, —Br, or —I.

In embodiments, R²⁰ is not —C(O)CH₃. In embodiments, R²⁰ is not —C(O)CH₂CH₃. In embodiments, R²⁰ is not —C(O)CH(CH₃)₂. In embodiments, R²⁰ is not unsubstituted methyl. In embodiments, R²⁰ is not —C(O)N(CH₃)₂. In embodiments, R²⁰ is not —CN. In embodiments, R²⁰ is not unsubstituted methoxy. In embodiments, R²⁰ is not unsubstituted tert-butyl. In embodiments, R²⁰ is not —OH. In embodiments, R²⁰ is not unsubstituted ethoxy. In embodiments, R²⁰ is not —N(CH₃)₂. In embodiments, R²⁰ is not —SH. In embodiments, R²⁰ is not —SCH₃. In embodiments, R²⁰ is not —SCH₂CH₃. In embodiments, R²⁰ is not unsubstituted ethyl. In embodiments, R²⁰ is not unsubstituted propyl. In embodiments, R²⁰ is not unsubstituted isopropyl. In embodiments, R²⁰ is not unsubstituted butyl. In embodiments, R²⁰ is not unsubstituted isobutyl. In embodiments, R²⁰ is not —NH₂. In embodiments, R²⁰ is not —NHCH₃. In embodiments, R²⁰ is not —NHCH₂CH₃. In embodiments, R²⁰ is not —N(CH₂CH₃)₂. In embodiments, R²⁰ is not —N(CH₃)(CH₂CH₃). In embodiments, R²⁰ is not halogen. In embodiments, R²⁰ is not —F. In embodiments, R²⁰ is not —Cl. In embodiments, R²⁰ is not —I. In embodiments, R²⁰ is not —Br. In embodiments, R²⁰ is not —C(O)NH₂. In embodiments, R²⁰ is not —C(O)NHCH₃. In embodiments, R²⁰ is not —C(O)NHCH₂CH₃. In embodiments, R²⁰ is not —C(O)N(CH₂CH₃)₂. In embodiments, R²⁰ is not —C(O)N(CH₃)(CH₂CH₃).

In embodiments, R²⁰ is not independently oxo, halogen, —CX²⁰₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX²⁰₃, —OCHX²⁰₂, R²¹-substituted or unsubstituted alkyl (e.g., C₁-C₈, C₁-C₆, or C₁-C₄), R²¹-substituted or unsubstituted heteroalkyl (e.g., 2 to 8 membered, 2 to 6 membered, or 2 to 4 membered), R²¹-substituted or unsubstituted cycloalkyl (e.g., C₃-C₈, C₃-C₆, or C₅-C₆), R²¹-substituted or unsubstituted heterocycloalkyl (e.g., 3 to 8 membered, 3 to 6 membered, or 5 to 6 membered), R²¹-substituted or unsubstituted aryl (e.g., C₆-C₁₀, C₁₀, or phenyl), or R²¹-substituted or unsubstituted heteroaryl (e.g., 5 to 10 membered, 5 to 9 membered, or 5 to 6 membered). X²⁰

In embodiments, R²¹ is not independently oxo, halogen, —CX²¹₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX²¹₃, —OCHX²¹₂, R²²-substituted or unsubstituted alkyl (e.g., C₁-C₈, C₁-C₆, or C₁-C₄), R²²-substituted or unsubstituted heteroalkyl (e.g., 2 to 8 membered, 2 to 6 membered, or 2 to 4 membered), R²²-substituted or unsubstituted cycloalkyl (e.g., C₃-C₈, C₃-C₆, or C₅-C₆), R²²-substituted or unsubstituted heterocycloalkyl (e.g., 3 to 8 membered, 3 to 6 membered, or 5 to 6 membered), R²²-substituted or unsubstituted aryl (e.g., C₆-C₁₀, C₁₀, or phenyl), or R²²-substituted or unsubstituted heteroaryl (e.g., 5 to 10 membered, 5 to 9 membered, or 5 to 6 membered). X²¹ is not —F, —Cl, —Br, or —I.

In embodiments, R²² is not independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, R¹ is not R²⁰-substituted or unsubstituted aryl or substituted or unsubstituted heteroaryl. In embodiments, R¹ is not R²⁰-substituted aryl. In embodiments, R¹ is not unsubstituted aryl. In embodiments, R¹ is not R²⁰-substituted C₆-C₁₀ aryl. In embodiments, R¹ is not unsubstituted C₆-C₁₀ aryl. In embodiments, R¹ is not R²⁰-substituted phenyl. In embodiments, R¹ is not unsubstituted phenyl. In embodiments, R¹ is not R²⁰-substituted heteroaryl. In embodiments, R¹ is not unsubstituted heteroaryl. In embodiments, R¹ is not R²⁰-substituted 5 to 10 membered heteroaryl. In embodiments, R¹ is not R²⁰-substituted 5 to 9 membered heteroaryl. In embodiments, R¹ is not unsubstituted 5 to 10 membered heteroaryl. In embodiments, R¹ is not unsubstituted 5 to 9 membered heteroaryl. In embodiments, R¹ is not R²⁰-substituted 5 to 6 membered heteroaryl. In embodiments, R¹ is not unsubstituted 5 to 6 membered heteroaryl. In embodiments, R¹ is not R²⁰-substituted 9 membered heteroaryl. In embodiments, R¹ is not R²⁰-substituted 10 membered heteroaryl. In embodiments, R¹ is not unsubstituted 9 membered heteroaryl. In embodiments, R¹ is not unsubstituted 10 membered heteroaryl. In embodiments, R¹ is not R²⁰-substituted 5 membered heteroaryl. In embodiments, R¹ is not R²⁰-substituted 6 membered heteroaryl. In embodiments, R¹ is not unsubstituted 5 membered heteroaryl. In embodiments, R¹ is not unsubstituted 6 membered heteroaryl.

In embodiments, R¹ is not R²⁰-substituted or unsubstituted pyrazolyl. In embodiments, R¹ is not R²⁰-substituted or unsubstituted pyridyl. In embodiments, R¹ is not R²⁰-substituted or unsubstituted imidazolyl. In embodiments, R¹ is not R²⁰-substituted or unsubstituted oxazolyl. In embodiments, R¹ is not R²⁰-substituted or unsubstituted isoxazolyl. In embodiments, R¹ is not R²⁰-substituted or unsubstituted thiazolyl. In embodiments, R¹ is not R²⁰-substituted or unsubstituted furanyl. In embodiments, R¹ is not R²⁰-substituted or unsubstituted pyrrolyl. In embodiments, R¹ is not R²⁰-substituted or unsubstituted thienyl. In embodiments, R¹ is not ortho-R²⁰-substituted phenyl. In embodiments, R¹ is not meta-R²⁰-substituted phenyl. In embodiments, R¹ is not para-R²⁰-substituted phenyl. In embodiments, R¹ is not ortho-halo-substituted phenyl. In embodiments, R¹ is not ortho-F-substituted phenyl. In embodiments, R¹ is not meta-C(O)CH₃-substituted phenyl.

In embodiments, R¹ is not R²⁰-substituted pyrazolyl. In embodiments, R¹ is not R²⁰-substituted pyridyl. In embodiments, R¹ is not R²⁰-substituted imidazolyl. In embodiments, R¹ is not R²⁰-substituted oxazolyl. In embodiments, R¹ is not R²⁰-substituted isoxazolyl. In embodiments, R¹ is not R²⁰-substituted thiazolyl. In embodiments, R¹ is not R²⁰-substituted furanyl. In embodiments, R¹ is not R²⁰-substituted pyrrolyl. In embodiments, R¹ is not R²⁰-substituted thienyl. In embodiments, R¹ is not unsubstituted pyrazolyl. In embodiments, R¹ is not unsubstituted pyridyl. In embodiments, R¹ is not unsubstituted imidazolyl. In embodiments, R¹ is not unsubstituted oxazolyl. In embodiments, R¹ is not unsubstituted isoxazolyl. In embodiments, R¹ is not unsubstituted thiazolyl. In embodiments, R¹ is not unsubstituted furanyl. In embodiments, R¹ is not unsubstituted pyrrolyl. In embodiments, R¹ is not unsubstituted thienyl.

In embodiments, R² is not hydrogen.

In embodiments, R³ is independently not halogen. In embodiments, R³ is independently not —CX³₃. In embodiments, R³ is independently not —CHX³₂. In embodiments, R³ is independently not —CH₂X³. In embodiments, R³ is independently not —OCX³₃. In embodiments, R³ is independently not —OCH₂X³. In embodiments, R³ is independently not —OCHX³₂. In embodiments, R³ is independently not —CN. In embodiments, R³ is independently not —SOₙ₃R^{3D}. In embodiments, R³ is independently not —SOᵥ₃NR^{3A}R^{3B}. In embodiments, R³ is independently not —NHC(O)NR^{3A}R^{3B}. In embodiments, R³ is independently not —N(O)ₘ₃. In embodiments, R³ is independently not —NR^{3A}R^{3B}. In embodiments, R³ is independently not —C(O)R^{3C}. In embodiments, R³ is independently not —C(O)—OR^{3C}. In embodiments, R³ is independently not —C(O)NR^{3A}R^{3B}. In embodiments, R³ is independently not —OR^{3D}. In embodiments, R³ is independently not —NR^{3A}SO₂R^{3D}. In embodiments, R³ is independently not —NR^{3A}C(O)R^{3C}. In embodiments, R³ is independently not —NR^{3A}C(O)OR^{3C}. In embodiments, R³ is independently not —NR^{3A}OR^{3C}. In embodiments, R³ is independently not —OH. In embodiments, R³ is independently not —NH₂. In embodiments, R³ is independently not —COOH. In embodiments, R³ is independently not —CONH₂. In embodiments, R³ is independently not —NO₂. In embodiments, R³ is independently not —SH. In embodiments, R³ is independently not —SO₂NR^{3A}R^{3B}. In embodiments, R³ is independently not meta—OCH₃ (relative to the bond to the remainder to the amine bonded to the pyrazolopyrimidine or pyrrolopyrimidine). In embodiments, R³ is independently not ortho unsubstituted phenyl (relative to the bond to the remainder to the amine bonded to the pyrazolopyrimidine or pyrrolopyrimidine).

In embodiments, R³ is independently not substituted or unsubstituted alkyl (e.g., C₁-C₈, C₁-C₆, or C₁-C₄). In embodiments, R³ is independently not substituted alkyl (e.g., C₁-C₈, C₁-C₆, or C₁-C₄). In embodiments, R³ is independently not unsubstituted alkyl (e.g., C₁-C₈, C₁-C₆, or C₁-C₄). In embodiments, R³ is independently not substituted or unsubstituted heteroalkyl (e.g., 2 to 8 membered, 2 to 6 membered, or 2 to 4 membered). In embodiments, R³ is independently not substituted heteroalkyl (e.g., 2 to 8 membered, 2 to 6 membered, or 2 to 4 membered). In embodiments, R³ is independently not unsubstituted heteroalkyl (e.g., 2 to 8 membered, 2 to 6 membered, or 2 to 4 membered). In embodiments, R³ is independently not substituted or unsubstituted cycloalkyl (e.g., C₃-C₈, C₃-C₆, or C₅-C₆). In embodiments, R³ is independently not substituted cycloalkyl (e.g., C₃-C₈, C₃-C₆, or C₅-C₆). In embodiments, R³ is independently not unsubstituted cycloalkyl (e.g., C₃-C₈, C₃-C₆, or C₅-C₆). In embodiments, R³ is independently not substituted or unsubstituted heterocycloalkyl (e.g., 3 to 8 membered, 3 to 6 membered, or 5 to 6 membered). In embodiments, R³ is independently not substituted heterocycloalkyl (e.g., 3 to 8 membered, 3 to 6 membered, or 5 to 6 membered). In embodiments, R³ is independently not unsubstituted heterocycloalkyl (e.g., 3 to 8 membered, 3 to 6 membered, or 5 to 6 membered). In embodiments, R³ is independently not substituted or unsubstituted aryl (e.g., C₆-C₁₀, C₁₀, or phenyl). In embodiments, R³ is independently not substituted aryl (e.g., C₆-C₁₀, C₁₀, or phenyl). In embodiments, R³ is independently not unsubstituted aryl (e.g., C₆-C₁₀, C₁₀, or phenyl). In embodiments, R³ is independently not substituted or unsubstituted heteroaryl (e.g., 5 to 10 membered, 5 to 9 membered, or 5 to 6 membered). In embodiments, R³ is independently not substituted heteroaryl (e.g., 5 to 10 membered, 5 to 9 membered, or 5 to 6 membered). In embodiments, R³ is independently not unsubstituted heteroaryl (e.g., 5 to 10 membered, 5 to 9 membered, or 5 to 6 membered).

In embodiments, R^{3A} is independently not hydrogen. In embodiments, R^{3A} is independently not —CX^{3A}₃. In embodiments, R^{3A} is independently not —CHX^{3A}₂. In embodiments, R^{3A} is independently not —CH₂X^{3A}. In embodiments, R^{3A} is independently not —CN. In embodiments, R^{3A} is independently not —COOH. In embodiments, R^{3A} is independently not —CONH₂.

In embodiments, R^{3A} is independently not substituted or unsubstituted alkyl (e.g., C₁-C₈, C₁-C₆, or C₁-C₄). In embodiments, R^{3A} is independently not substituted alkyl (e.g., C₁-C₈, C₁-C₆, or C₁-C₄). In embodiments, R^{3A} is independently not unsubstituted alkyl (e.g., C₁-C₈, C₁-C₆, or C₁-C₄). In embodiments, R^{3A} is independently not substituted or unsubstituted heteroalkyl (e.g., 2 to 8 membered, 2 to 6 membered, or 2 to 4 membered). In embodiments, R^{3A} is independently not substituted heteroalkyl (e.g., 2 to 8 membered, 2 to 6 membered, or 2 to 4 membered). In embodiments, R^{3A} is independently not unsubstituted heteroalkyl (e.g., 2 to 8 membered, 2 to 6 membered, or 2 to 4 membered). In embodiments, R^{3A} is independently not substituted or unsubstituted cycloalkyl (e.g., C₃-C₈, C₃-C₆, or C₅-C₆). In embodiments, R^{3A} is independently not substituted cycloalkyl (e.g., C₃-C₈, C₃-C₆, or C₅-C₆). In embodiments, R^{3A} is independently not unsubstituted cycloalkyl (e.g., C₃-C₈, C₃-C₆, or C₅-C₆). In embodiments, R^{3A} is independently not substituted or unsubstituted heterocycloalkyl (e.g., 3 to 8 membered, 3 to 6 membered, or 5 to 6 membered). In embodiments, R^{3A} is independently not substituted heterocycloalkyl (e.g., 3 to 8 membered, 3 to 6 membered, or 5 to 6 membered). In embodiments, R^{3A} is independently not unsubstituted heterocycloalkyl (e.g., 3 to 8 membered, 3 to 6 membered, or 5 to 6 membered). In embodiments, R^{3A} is independently not substituted or unsubstituted aryl (e.g., C₆-C₁₀, C₁₀, or phenyl). In embodiments, R^{3A} is independently not substituted aryl (e.g., C₆-C₁₀, C₁₀, or phenyl). In embodiments, R^{3A} is independently not unsubstituted aryl (e.g., C₆-C₁₀, C₁₀, or phenyl). In embodiments, R^{3A} is independently not substituted or unsubstituted heteroaryl (e.g., 5 to 10 membered, 5 to 9 membered, or 5 to 6 membered). In embodiments, R^{3A} is independently not substituted heteroaryl (e.g., 5 to 10 membered, 5 to 9 membered, or 5 to 6 membered). In embodiments, R^{3A} is independently not unsubstituted heteroaryl (e.g., 5 to 10 membered, 5 to 9 membered, or 5 to 6 membered). In embodiments, R^{3A} is independently not unsubstituted methyl. In embodiments, R^{3A} is independently not unsubstituted ethyl. In embodiments, R^{3A} is independently not unsubstituted propyl. In embodiments, R^{3A} is independently not unsubstituted isopropyl. In embodiments, R^{3A} is independently not unsubstituted tert-butyl.

In embodiments, R^{3B} is independently not hydrogen. In embodiments, R^{3B} is independently not —CX^{3B}₃. In embodiments, R^{3B} is independently not —CHX^{3B}₂. In embodiments, R^{3B} is independently not —CH₂X^{3B}. In embodiments, R^{3B} is independently not —CN. In embodiments, R^{3B} is independently not —COOH. In embodiments, R^{3B} is independently not —CONH₂.

In embodiments, R^{3B} is independently not substituted or unsubstituted alkyl (e.g., C₁-C₈, C₁-C₆, or C₁-C₄). In embodiments, R^{3B} is independently not substituted alkyl (e.g., C₁-C₈, C₁-C₆, or C₁-C₄). In embodiments, R^{3B} is independently not unsubstituted alkyl (e.g., C₁-C₈, C₁-C₆, or C₁-C₄). In embodiments, R^{3B} is independently not substituted or unsubstituted heteroalkyl (e.g., 2 to 8 membered, 2 to 6 membered, or 2 to 4 membered). In embodiments, R^{3B} is independently not substituted heteroalkyl (e.g., 2 to 8 membered, 2 to 6 membered, or 2 to 4 membered). In embodiments, R^{3B} is independently not unsubstituted heteroalkyl (e.g., 2 to 8 membered, 2 to 6 membered, or 2 to 4 membered). In embodiments, R^{3B} is independently not substituted or unsubstituted cycloalkyl (e.g., C₃-C₈, C₃-C₆, or C₅-C₆). In embodiments, R^{3B} is independently not substituted cycloalkyl (e.g., C₃-C₈, C₃-C₆, or C₅-C₆). In embodiments, R^{3B} is independently not unsubstituted cycloalkyl (e.g., C₃-C₈, C₃-C₆, or C₅-C₆). In embodiments, R^{3B} is independently not substituted or unsubstituted heterocycloalkyl (e.g., 3 to 8 membered, 3 to 6 membered, or 5 to 6 membered). In embodiments, R^{3B} is independently not substituted heterocycloalkyl (e.g., 3 to 8 membered, 3 to 6 membered, or 5 to 6 membered). In embodiments, R^{3B} is independently not unsubstituted heterocycloalkyl (e.g., 3 to 8 membered, 3 to 6 membered, or 5 to 6 membered). In embodiments, R^{3B} is independently not substituted or unsubstituted aryl (e.g., C₆-C₁₀, C₁₀, or phenyl). In embodiments, R^{3B} is independently not substituted aryl (e.g., C₆-C₁₀, C₁₀, or phenyl). In embodiments, R^{3B} is independently not unsubstituted aryl (e.g., C₆-C₁₀, C₁₀, or phenyl). In embodiments, R^{3B} is independently not substituted or unsubstituted heteroaryl (e.g., 5 to 10 membered, 5 to 9 membered, or 5 to 6 membered). In embodiments, R^{3B} is independently not substituted heteroaryl (e.g., 5 to 10 membered, 5 to 9 membered, or 5 to 6 membered). In embodiments, R^{3B} is independently not unsubstituted heteroaryl (e.g., 5 to 10 membered, 5 to 9 membered, or 5 to 6 membered). In embodiments, R^{3B} is independently not unsubstituted methyl. In embodiments, R^{3B} is independently not unsubstituted ethyl. In embodiments, R^{3B} is independently not unsubstituted propyl. In embodiments, R^{3B} is independently not unsubstituted isopropyl. In embodiments, R^{3B} is independently not unsubstituted tert-butyl.

In embodiments, R^{3C} is independently not hydrogen. In embodiments, R^{3C} is independently not —CX^{3C}₃. In embodiments, R^{3C} is independently not —CHX^{3C}₂. In embodiments, R^{3C} is independently not —CH₂X^{3C}. In embodiments, R^{3C} is independently not —CN. In embodiments, R^{3C} is independently not —COOH. In embodiments, R^{3C} is independently not —CONH₂.

In embodiments, R^{3C} is independently not substituted or unsubstituted alkyl (e.g., C₁-C₈, C₁-C₆, or C₁-C₄). In embodiments, R^{3C} is independently not substituted alkyl (e.g., C₁-C₈, C₁-C₆, or C₁-C₄). In embodiments, R^{3C} is independently not unsubstituted alkyl (e.g., C₁-C₈, C₁-C₆, or C₁-C₄). In embodiments, R^{3C} is independently not substituted or unsubstituted heteroalkyl (e.g., 2 to 8 membered, 2 to 6 membered, or 2 to 4 membered). In embodiments, R³ is independently not substituted heteroalkyl (e.g., 2 to 8 membered, 2 to 6 membered, or 2 to 4 membered). In embodiments, R^{3C} is independently not unsubstituted heteroalkyl (e.g., 2 to 8 membered, 2 to 6 membered, or 2 to 4 membered). In embodiments, R^{3C} is independently not substituted or unsubstituted cycloalkyl (e.g., C₃-C₈, C₃-C₆, or C₅-C₆). In embodiments, R^{3C} is independently not substituted cycloalkyl (e.g., C₃-C₈, C₃-C₆, or C₅-C₆). In embodiments, R^{3C} is independently not unsubstituted cycloalkyl (e.g., C₃-C₈, C₃-C₆, or C₅-C₆). In embodiments, R^{3C} is independently not substituted or unsubstituted heterocycloalkyl (e.g., 3 to 8 membered, 3 to 6 membered, or 5 to 6 membered). In embodiments, R^{3C} is independently not substituted heterocycloalkyl (e.g., 3 to 8 membered, 3 to 6 membered, or 5 to 6 membered). In embodiments, R^{3C} is independently not unsubstituted heterocycloalkyl (e.g., 3 to 8 membered, 3 to 6 membered, or 5 to 6 membered). In embodiments, R^{3C} is independently not substituted or unsubstituted aryl (e.g., C₆-C₁₀, C₁₀, or phenyl). In embodiments, R^{3C} is independently not substituted aryl (e.g., C₆-C₁₀, C₁₀, or phenyl). In embodiments, R^{3C} is independently not unsubstituted aryl (e.g., C₆-C₁₀, C₁₀, or phenyl). In embodiments, R^{3C} is independently not substituted or unsubstituted heteroaryl (e.g., 5 to 10 membered, 5 to 9 membered, or 5 to 6 membered). In embodiments, R^{3C} is independently not substituted heteroaryl (e.g., 5 to 10 membered, 5 to 9 membered, or 5 to 6 membered). In embodiments, R^{3C} is independently not unsubstituted heteroaryl (e.g., 5 to 10 membered, 5 to 9 membered, or 5 to 6 membered). In embodiments, R^{3C} is independently not unsubstituted methyl. In embodiments, R^{3C} is independently not unsubstituted ethyl. In embodiments, R^{3C} is independently not unsubstituted propyl. In embodiments, R^{3C} is independently not unsubstituted isopropyl. In embodiments, R³ is independently not unsubstituted tert-butyl.

In embodiments, R^{3D} is independently not hydrogen. In embodiments, R^{3D} is independently not —CX^{3D}₃. In embodiments, R^{3D} is independently not —CHX^{3D}₂. In embodiments, R^{3D} is independently not —CH₂X^{3D}. In embodiments, R^{3D} is independently not —CN. In embodiments, R^{3D} is independently not —COOH. In embodiments, R^{3D} is independently not —CONH₂.

In embodiments, R^{3D} is independently not substituted or unsubstituted alkyl (e.g., C₁-C₈, C₁-C₆, or C₁-C₄). In embodiments, R^{3D} is independently not substituted alkyl (e.g., C₁-C₈, C₁-C₆, or C₁-C₄). In embodiments, R^{3D} is independently not unsubstituted alkyl (e.g., C₁-C₈, C₁-C₆, or C₁-C₄). In embodiments, R^{3D} is independently not substituted or unsubstituted heteroalkyl (e.g., 2 to 8 membered, 2 to 6 membered, or 2 to 4 membered). In embodiments, R^{3D} is independently not substituted heteroalkyl (e.g., 2 to 8 membered, 2 to 6 membered, or 2 to 4 membered). In embodiments, R^{3D} is independently not unsubstituted heteroalkyl (e.g., 2 to 8 membered, 2 to 6 membered, or 2 to 4 membered). In embodiments, R^{3D} is independently not substituted or unsubstituted cycloalkyl (e.g., C₃-C₈, C₃-C₆, or C₅-C₆). In embodiments, R^{3D} is independently not substituted cycloalkyl (e.g., C₃-C₈, C₃-C₆, or C₅-C₆). In embodiments, R^{3D} is independently not unsubstituted cycloalkyl (e.g., C₃-C₈, C₃-C₆, or C₅-C₆). In embodiments, R^{3D} is independently not substituted or unsubstituted heterocycloalkyl (e.g., 3 to 8 membered, 3 to 6 membered, or 5 to 6 membered). In embodiments, R^{3D} is independently not substituted heterocycloalkyl (e.g., 3 to 8 membered, 3 to 6 membered, or 5 to 6 membered). In embodiments, R^{3D} is independently not unsubstituted heterocycloalkyl (e.g., 3 to 8 membered, 3 to 6 membered, or 5 to 6 membered). In embodiments, R^{3D} is independently not substituted or unsubstituted aryl (e.g., C₆-C₁₀, C₁₀, or phenyl). In embodiments, R^{3D} is independently not substituted aryl (e.g., C₆-C₁₀, C₁₀, or phenyl). In embodiments, R^{3D} is independently not unsubstituted aryl (e.g., C₆-C₁₀, C₁₀, or phenyl). In embodiments, R^{3D} is independently not substituted or unsubstituted heteroaryl (e.g., 5 to 10 membered, 5 to 9 membered, or 5 to 6 membered). In embodiments, R^{3D} is independently not substituted heteroaryl (e.g., 5 to 10 membered, 5 to 9 membered, or 5 to 6 membered). In embodiments, R^{3D} is independently not unsubstituted heteroaryl (e.g., 5 to 10 membered, 5 to 9 membered, or 5 to 6 membered). In embodiments, R^{3D} is independently not unsubstituted methyl. In embodiments, R^{3D} is independently not unsubstituted ethyl. In embodiments, R^{3D} is independently not unsubstituted propyl. In embodiments, R^{3D} is independently not unsubstituted isopropyl. In embodiments, R^{3D} is independently not unsubstituted tert-butyl.

In embodiments, R³ is independently not hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, R²⁶-substituted or unsubstituted alkyl, R²⁶-substituted or unsubstituted heteroalkyl, R²⁶-substituted or unsubstituted cycloalkyl, R²⁶-substituted or unsubstituted heterocycloalkyl, R²⁶-substituted or unsubstituted aryl, or R²⁶-substituted or unsubstituted heteroaryl. In embodiments, R³ is independently not halogen, —CX³₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX³₃, —OCHX³₂, R²⁶-substituted or unsubstituted alkyl, R²⁶-substituted or unsubstituted heteroalkyl, R²⁶-substituted or unsubstituted cycloalkyl, R²⁶-substituted or unsubstituted heterocycloalkyl, R²⁶-substituted or unsubstituted aryl, or R²⁶-substituted or unsubstituted heteroaryl. In embodiments, R³ is independently not halogen, —CX³₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX³₃, —OCHX³₂, R²⁶-substituted or unsubstituted C₁-C₈ alkyl, R²⁶-substituted or unsubstituted 2 to 8 membered heteroalkyl, R²⁶-substituted or unsubstituted C₃-C₈ cycloalkyl, R²⁶-substituted or unsubstituted 3 to 6 membered heterocycloalkyl, R²⁶-substituted or unsubstituted phenyl, or R²⁶-substituted or unsubstituted 5 to 6 membered heteroaryl. In embodiments, X³ is not —F, —Cl, —Br, or —I. In embodiments, R³ is independently not hydrogen. In embodiments, R³ is independently not methyl. In embodiments, R³ is independently not ethyl.

In embodiments, R²⁶ is independently not oxo, halogen, —CX²⁶₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX²⁶₃, —OCHX²⁶₂, R²⁷-substituted or unsubstituted alkyl (e.g., C₁-C₈, C₁-C₆, or C₁-C₄), R²⁷-substituted or unsubstituted heteroalkyl (e.g., 2 to 8 membered, 2 to 6 membered, or 2 to 4 membered), R²⁷-substituted or unsubstituted cycloalkyl (e.g., C₃-C₈, C₃-C₆, or C₅-C₆), R²⁷-substituted or unsubstituted heterocycloalkyl (e.g., 3 to 8 membered, 3 to 6 membered, or 5 to 6 membered), R²⁷-substituted or unsubstituted aryl (e.g., C₆-C₁₀, C₁₀, or phenyl), or R²⁷-substituted or unsubstituted heteroaryl (e.g., 5 to 10 membered, 5 to 9 membered, or 5 to 6 membered). In embodiments, X²⁶ is not —F, —Cl, —Br, or —I.

In embodiments, R²⁷ is independently not oxo, halogen, —CX²⁷₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX²⁷₃, —OCHX²⁷₂, R²⁸-substituted or unsubstituted alkyl (e.g., C₁-C₈, C₁-C₆, or C₁-C₄), R²⁸-substituted or unsubstituted heteroalkyl (e.g., 2 to 8 membered, 2 to 6 membered, or 2 to 4 membered), R²⁸-substituted or unsubstituted cycloalkyl (e.g., C₃-C₈, C₃-C₆, or C₅-C₆), R²⁸-substituted or unsubstituted heterocycloalkyl (e.g., 3 to 8 membered, 3 to 6 membered, or 5 to 6 membered), R²⁸-substituted or unsubstituted aryl (e.g., C₆-C₁₀, C₁₀, or phenyl), or R²⁸-substituted or unsubstituted heteroaryl (e.g., 5 to 10 membered, 5 to 9 membered, or 5 to 6 membered). In embodiments, X²⁷ is not —F, —Cl, —Br, or —I.

In embodiments, R^{3A} is independently not hydrogen, —CX^{3A}₃, —CN, —COOH, —CONH₂, —CHX^{3A}₂, —CH₂X^{3A}, R^{26A}-substituted or unsubstituted alkyl, R^{26A}-substituted or unsubstituted heteroalkyl, R^{26A}-substituted or unsubstituted cycloalkyl, R^{26A}-substituted or unsubstituted heterocycloalkyl, R^{26A}-substituted or unsubstituted aryl, or R^{26A}-substituted or unsubstituted heteroaryl. In embodiments, R^{3A} is independently not hydrogen, —CX^{3A}₃, —CN, —COOH, —CONH₂, —CHX^{3A}₂, —CH₂X^{3A}, R^{26A}-substituted or unsubstituted C₁-C₈ alkyl, R^{26A}-substituted or unsubstituted 2 to 8 membered heteroalkyl, R^{26A}-substituted or unsubstituted C₃-C₈ cycloalkyl, R^{26A}-substituted or unsubstituted 3 to 6 membered heterocycloalkyl, R^{26A}-substituted or unsubstituted phenyl, or R^{26A}-substituted or unsubstituted 5 to 6 membered heteroaryl. In embodiments, X^{3A} is not —F, —Cl, —Br, or —I. In embodiments, R^{3A} is independently not hydrogen. In embodiments, R^{3A} is independently not methyl. In embodiments, R^{3A} is independently not ethyl.

In embodiments, R^{26A} is independently not oxo, halogen, —CX^{26A}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{26A}₃, —OCHX^{26A}₂, R^{27A}-substituted or unsubstituted alkyl (e.g., C₁-C₈, C₁-C₆, or C₁-C₄), R^{27A}-substituted or unsubstituted heteroalkyl (e.g., 2 to 8 membered, 2 to 6 membered, or 2 to 4 membered), R^{27A}-substituted or unsubstituted cycloalkyl (e.g., C₃-C₈, C₃-C₆, or C₅-C₆), R^{27A}-substituted or unsubstituted heterocycloalkyl (e.g., 3 to 8 membered, 3 to 6 membered, or 5 to 6 membered), R^{27A}-substituted or unsubstituted aryl (e.g., C₆-C₁₀, C₁₀, or phenyl), or R^{27A}-substituted or unsubstituted heteroaryl (e.g., 5 to 10 membered, 5 to 9 membered, or 5 to 6 membered). In embodiments, X^{26A} is not —F, —Cl, —Br, or —I.

In embodiments, R^{27A} is independently not oxo, halogen, —CX^{27A}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{27A}₃, —OCHX^{27A}₂, R^{28A}-substituted or unsubstituted alkyl (e.g., C₁-C₈, C₁-C₆, or C₁-C₄), R^{28A}-substituted or unsubstituted heteroalkyl (e.g., 2 to 8 membered, 2 to 6 membered, or 2 to 4 membered), R^{28A}-substituted or unsubstituted cycloalkyl (e.g., C₃-C₈, C₃-C₆, or C₅-C₆), R^{28A}-substituted or unsubstituted heterocycloalkyl (e.g., 3 to 8 membered, 3 to 6 membered, or 5 to 6 membered), R^{28A}-substituted or unsubstituted aryl (e.g., C₆-C₁₀, C₁₀, or phenyl), or R^{28A}-substituted or unsubstituted heteroaryl (e.g., 5 to 10 membered, 5 to 9 membered, or 5 to 6 membered). In embodiments, X^{27A} is not —F, —Cl, —Br, or —I.

In embodiments, R^{3B} is independently not hydrogen, —CX^{3B}₃, —CN, —COOH, —CONH₂, —CHX^{3B}₂, —CH₂X^{3B}, R^{26B}-substituted or unsubstituted alkyl, R^{26B}-substituted or unsubstituted heteroalkyl, R^{26B}-substituted or unsubstituted cycloalkyl, R^{26B}-substituted or unsubstituted heterocycloalkyl, R^{26B}-substituted or unsubstituted aryl, or R^{26B}-substituted or unsubstituted heteroaryl. In embodiments, R^{3B} is independently not hydrogen, —CX^{3B}₃, —CN, —COOH, —CONH₂, —CHX^{3B}₂, —CH₂X^{3B}, R^{26B}-substituted or unsubstituted C₁-C₈ alkyl, R^{26B}-substituted or unsubstituted 2 to 8 membered heteroalkyl, R^{26B}-substituted or unsubstituted C₃-C₈ cycloalkyl, R^{26B}-substituted or unsubstituted 3 to 6 membered heterocycloalkyl, R^{26B}-substituted or unsubstituted phenyl, or R^{26B}-substituted or unsubstituted 5 to 6 membered heteroaryl. In embodiments, X^{3B} is not —F, —Cl, —Br, or —I. In embodiments, R^{3B} is independently not hydrogen. In embodiments, R^{3B} is independently not methyl. In embodiments, R^{3B} is independently not ethyl.

In embodiments, R^{26B} is independently not oxo, halogen, —CX^{26B}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{26B}₃, —OCHX^{26B}₂, R^{27B}-substituted or unsubstituted alkyl (e.g., C₁-C₈, C₁-C₆, or C₁-C₄), R^{27B}-substituted or unsubstituted heteroalkyl (e.g., 2 to 8 membered, 2 to 6 membered, or 2 to 4 membered), R^{27B}-substituted or unsubstituted cycloalkyl (e.g., C₃-C₈, C₃-C₆, or C₅-C₆), R^{27B}-substituted or unsubstituted heterocycloalkyl (e.g., 3 to 8 membered, 3 to 6 membered, or 5 to 6 membered), R^{27B}-substituted or unsubstituted aryl (e.g., C₆-C₁₀, C₁₀, or phenyl), or R^{27B}-substituted or unsubstituted heteroaryl (e.g., 5 to 10 membered, 5 to 9 membered, or 5 to 6 membered). In embodiments, X^{26B} is not —F, —Cl, —Br, or —I.

In embodiments, R^{27B} is independently not oxo, halogen, —CX^{27B}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{27B}₃, —OCHX^{27B}₂, R^{28B}-substituted or unsubstituted alkyl (e.g., C₁-C₈, C₁-C₆, or C₁-C₄), R^{28B}-substituted or unsubstituted heteroalkyl (e.g., 2 to 8 membered, 2 to 6 membered, or 2 to 4 membered), R^{28B}-substituted or unsubstituted cycloalkyl (e.g., C₃-C₈, C₃-C₆, or C₅-C₆), R^{28B}-substituted or unsubstituted heterocycloalkyl (e.g., 3 to 8 membered, 3 to 6 membered, or 5 to 6 membered), R^{28B}-substituted or unsubstituted aryl (e.g., C₆-C₁₀, C₁₀, or phenyl), or R^{28B}-substituted or unsubstituted heteroaryl (e.g., 5 to 10 membered, 5 to 9 membered, or 5 to 6 membered). In embodiments, X^{27B} is not —F, —Cl, —Br, or —I.

In embodiments, R^{3C} is independently not hydrogen, —CX^{3C}₃, —CN, —COOH, —CONH₂, —CHX^{3C}₂, —CH₂X^{3C}, R^{26C}-substituted or unsubstituted alkyl, R^{26C}-substituted or unsubstituted heteroalkyl, R^{26C}-substituted or unsubstituted cycloalkyl, R^{26C}-substituted or unsubstituted heterocycloalkyl, R^{26C}-substituted or unsubstituted aryl, or R^{26C}-substituted or unsubstituted heteroaryl. In embodiments, R^{3C} is independently not hydrogen, —CX^{3C}₃, —CN, —COOH, —CONH₂, —CHX^{3C}₂, —CH₂X^{3C}, R^{26C}-substituted or unsubstituted C₁-C₈ alkyl, R^{26C}-substituted or unsubstituted 2 to 8 membered heteroalkyl, R^{26C}-substituted or unsubstituted C₃-C₈ cycloalkyl, R^{26C}-substituted or unsubstituted 3 to 6 membered heterocycloalkyl, R^{26C}-substituted or unsubstituted phenyl, or R^{26C}-substituted or unsubstituted 5 to 6 membered heteroaryl. In embodiments, X^{3C} is not —F, —Cl, —Br, or —I. In embodiments, R³ is independently not hydrogen. In embodiments, R^{3C} is independently not methyl. In embodiments, R^{3C} is independently not ethyl.

In embodiments, R^{26C} is independently not oxo, halogen, —CX^{26C}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{26C}₃, —OCHX^{26C}₂, R^{27C}-substituted or unsubstituted alkyl (e.g., C₁-C₈, C₁-C₆, or C₁-C₄), R^{27C}-substituted or unsubstituted heteroalkyl (e.g., 2 to 8 membered, 2 to 6 membered, or 2 to 4 membered), R^{27C}-substituted or unsubstituted cycloalkyl (e.g., C₃-C₈, C₃-C₆, or C₅-C₆), R^{27C}-substituted or unsubstituted heterocycloalkyl (e.g., 3 to 8 membered, 3 to 6 membered, or 5 to 6 membered), R^{27C}-substituted or unsubstituted aryl (e.g., C₆-C₁₀, C₁₀, or phenyl), or R^{27C}-substituted or unsubstituted heteroaryl (e.g., 5 to 10 membered, 5 to 9 membered, or 5 to 6 membered). In embodiments, X^{26C} is not —F, —Cl, —Br, or —I.

In embodiments, R^{27C} is independently not oxo, halogen, —CX^{27C}₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{27C}₃, —OCHX^{27C}₂, R^{28C}-substituted or unsubstituted alkyl (e.g., C₁-C₈, C₁-C₆, or C₁-C₄), R^{28C}-substituted or unsubstituted heteroalkyl (e.g., 2 to 8 membered, 2 to 6 membered, or 2 to 4 membered), R^{28C}-substituted or unsubstituted cycloalkyl (e.g., C₃-C₈, C₃-C₆, or C₅-C₆), R^{28C}-substituted or unsubstituted heterocycloalkyl (e.g., 3 to 8 membered, 3 to 6 membered, or 5 to 6 membered), R^{28C}-substituted or unsubstituted aryl (e.g., C₆-C₁₀, C₁₀, or phenyl), or R^{28C}-substituted or unsubstituted heteroaryl (e.g., 5 to 10 membered, 5 to 9 membered, or 5 to 6 membered). In embodiments, X^{27C} is not —F, —Cl, —Br, or —I.

In embodiments, R^{3D} is independently not hydrogen, —CX^{3D}₃, —CN, —COOH, —CONH₂, —CHX^{3D}₂, —CH₂X^{3D}, R^{26D}-substituted or unsubstituted alkyl, R^{26D}-substituted or unsubstituted heteroalkyl, R^{26D}-substituted or unsubstituted cycloalkyl, R^{26D}-substituted or unsubstituted heterocycloalkyl, R^{26D}-substituted or unsubstituted aryl, or R^{26D}-substituted or unsubstituted heteroaryl. In embodiments, R^{3D} is independently not hydrogen, —CX^{3D}₃, —CN, —COOH, —CONH₂, —CHX^{3D}₂, —CH₂X^{3D}, R^{26D}-substituted or unsubstituted C₁-C₈ alkyl, R^{26D}-substituted or unsubstituted 2 to 8 membered heteroalkyl, R^{26D}-substituted or unsubstituted C₃-C₈ cycloalkyl, R^{26D}-substituted or unsubstituted 3 to 6 membered heterocycloalkyl, R^{26D}-substituted or unsubstituted phenyl, or R^{26D}-substituted or unsubstituted 5 to 6 membered heteroaryl. In embodiments, X^{3D} is not —F, —Cl, —Br, or —I. In embodiments, R^{3D} is independently not hydrogen. In embodiments, R^{3D} is independently not methyl. In embodiments, R^{3D} is independently not ethyl.

In embodiments, R^{26D} is independently not oxo, halogen, —CX^{26D}₃, —CN, —OH, —NH₂, —COOH, —CONH₂, —NO₂, —SH, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{26D}₃, —OCHX^{26D}₂, R^{27D}-substituted or unsubstituted alkyl (e.g., C₁-C₈, C₁-C₆, or C₁-C₄), R^{27D}-substituted or unsubstituted heteroalkyl (e.g., 2 to 8 membered, 2 to 6 membered, or 2 to 4 membered), R^{27D}-substituted or unsubstituted cycloalkyl (e.g., C₃-C₈, C₃-C₆, or C₅-C₆), R^{27D}-substituted or unsubstituted heterocycloalkyl (e.g., 3 to 8 membered, 3 to 6 membered, or 5 to 6 membered), R^{27D}-substituted or unsubstituted aryl (e.g., C₆-C₁₀, C₁₀, or phenyl), or R^{27D}-substituted or unsubstituted heteroaryl (e.g., 5 to 10 membered, 5 to 9 membered, or 5 to 6 membered). In embodiments, X^{26D} is not —F, —Cl, —Br, or —I.

In embodiments, R^{27D} is independently not oxo, halogen, —CX^{27D}₃, —CN, —OH, —NH₂, —COOH, —CONH₂, —NO₂, —SH, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCX^{27D}₃, —OCHX^{27D}₂, R^{28D}-substituted or unsubstituted alkyl (e.g., C₁-C₈, C₁-C₆, or C₁-C₄), R^{28D}-substituted or unsubstituted heteroalkyl (e.g., 2 to 8 membered, 2 to 6 membered, or 2 to 4 membered), R^{28D}-substituted or unsubstituted cycloalkyl (e.g., C₃-C₈, C₃-C₆, or C₅-C₆), R^{28D}-substituted or unsubstituted heterocycloalkyl (e.g., 3 to 8 membered, 3 to 6 membered, or 5 to 6 membered), R^{28D}-substituted or unsubstituted aryl (e.g., C₆-C₁₀, C₁₀, or phenyl), or R^{28B}-substituted or unsubstituted heteroaryl (e.g., 5 to 10 membered, 5 to 9 membered, or 5 to 6 membered). In embodiments, X^{27D} is not —F, —Cl, —Br, or —I.

In embodiments, R²⁸, R^{28A}, R^{28B}, R^{28C}, and R^{28D} are independently not oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀, C₁₀, or phenyl), or unsubstituted heteroaryl (e.g., 5 to 10 membered, 5 to 9 membered, or 5 to 6 membered).

In embodiments, the compound is not

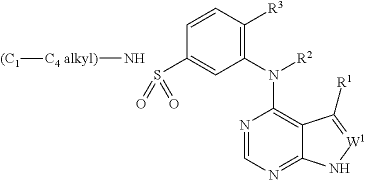

wherein R¹, R², R³, and W¹ are as described herein, including in an embodiment.

In embodiments, the compound is not

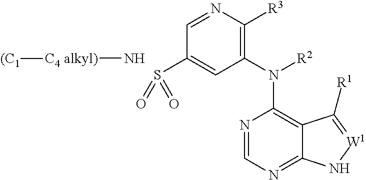

wherein R¹, R², R³, and W¹ are as described herein, including in an embodiment.

In embodiments, the compound is not

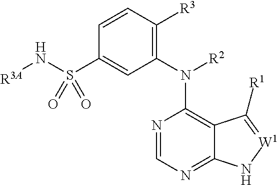

wherein R¹, R², R³, R^{3A}, and W¹ are as described herein, including in an embodiment.

In embodiments, the compound is not

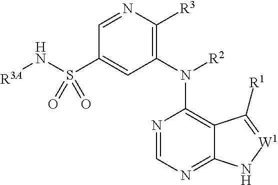

wherein R¹, R², R³, R^{3A}, and W¹ are as described herein, including in an embodiment.

In embodiments, the compound is not

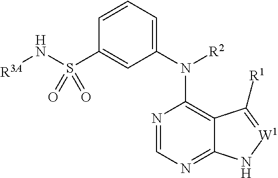

wherein R¹, R², R³, R^{3A}, and W¹ are as described herein, including in an embodiment.

In embodiments, the compound is not

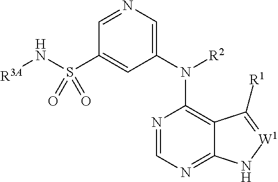

wherein R¹, R², R³, R^{3A}, and W¹ are as described herein, including in an embodiment.

In embodiments, the compound is not

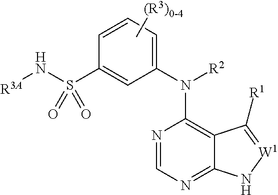

wherein R¹, R², R³, R^{3A}, and W¹ are as described herein, including in an embodiment.

In embodiments, the compound is not

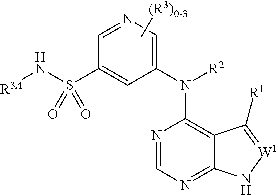

wherein R¹, R², R³, R^{3A}, and W¹ are as described herein, including in an embodiment.

In embodiments, the compound is not

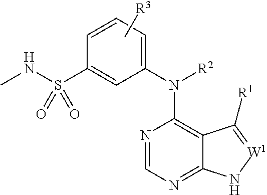

wherein R¹, R², R³, R^{3A}, and W¹ are as described herein, including in an embodiment.

C. Pharmaceutical Compositions

In another aspect is provided a pharmaceutical composition including a pharmaceutically acceptable excipient and a compound, or pharmaceutically acceptable salt thereof, as described herein, including embodiments.

In embodiments, the pharmaceutical composition includes the active ingredient (e.g., compound described herein or pharmaceutically acceptable salt thereof) in a therapeutically effective amount, i.e., in an amount effective to achieve its intended purpose. In embodiments, the therapeutically effective amount is administered in one dose. In embodiments, the therapeutically effective amount is administered in divided doses that combine to provide a therapeutically effective amount. In embodiments, the therapeutically effective amount is administered in a single dose that is administered multiple times over the course of a treatment. The actual amount effective for a particular application will depend, inter alia, on the condition being treated. When administered in methods to treat a disease, such compositions will contain an amount of active ingredient effective to achieve the desired result, e.g., inhibiting cell proliferation. In embodiments, the pharmaceutical composition includes an anti-cancer agent (i.e., an anti-cancer agent in addition to the compound described herein that is included in the pharmaceutical composition). In embodiments, the pharmaceutical composition includes an anti-cancer agent in an effective amount. In embodiments, the anti-cancer agent is an EGFR modulator, HER2 modulator, HER3 modulator, HER4 modulator, c-MET modulator, PI3K modulator, MEK modulator, MAPK modulator, RAF modulator, BRAF modulator, AKT modulator, RAS modulator, KRAS modulator, heregulin modulator, neuregulin modulator, or mTOR modulator. In embodiments, the anti-cancer agent is lapatinib, vemurafenib, or selumetinib. In embodiments, the anti-cancer agent is trastuzumab, trastuzumab emtansine, pertuzumab, tamoxifen, gefitinib, erlotinib, afatinib, brigatinib, icotinib, cetuximab, panitumumab, zalutumumab, nimotuzumab, matuzumab, or lapatinib.

D. Methods of Treatment

In an aspect is provided a method of treating cancer in a patient in need of such treatment, the method including administering a therapeutically effective amount of a compound, or a pharmaceutically acceptable salt thereof, described herein.

In embodiments, the cancer is lung cancer, non-small cell lung cancer, ovarian cancer, breast cancer, triple negative breast cancer, melanoma, head and neck cancer, colon cancer, gatric cancer, glioma, anal cancer, stomach cancer, uterine cancer, uterine serous endometrial carcinoma, salivary duct carcinoma, testicular cancer, esophageal cancer, or glioblastoma multiforme. In embodiments, the cancer is lung cancer, breast cancer, colorectal carcinoma, head and neck cancer, uveal melanoma, gastric cancer, ovarian cancer, prostate cancer, or bladder cancer. In embodiments, the cancer is lung cancer. In embodiments, the cancer is breast cancer. In embodiments, the cancer is colorectal carcinoma. In embodiments, the cancer is head and neck cancer. In embodiments, the cancer is uveal melanoma. In embodiments, the cancer is gastric cancer. In embodiments, the cancer is ovarian cancer. In embodiments, the cancer is prostate cancer. In embodiments, the cancer is bladder cancer. In embodiments, the cancer possesses an activating mutation in EGFR. In embodiments, the cancer possesses an activating mutation in HER2. In embodiments, the cancer possesses an activating mutation in HER3. In embodiments, the cancer possesses an activating mutation in HER4. The cancer is dependent on neuregulin signaling through HER2/HER3. In embodiments, the cancer overexpresses neuregulin. In embodiments, the cancer possesses a drug resistance mutation in EGFR. In embodiments, the cancer possesses a drug resistance mutation in HER2. In embodiments, the cancer possesses a drug resistance mutation in HER3. In embodiments, the cancer possesses a drug resistance mutation in HER4. In embodiments, the cancer is dependent on neuregulin signaling through HER2/HER3.

In embodiments, the cancer is resistant to a HER2 inhibitor. In embodiments, the cancer is resistant to an EGFR inhibitor. In embodiments, the cancer is a HER2 overexpressing cancer. In embodiments, the cancer is a HER2 positive cancer. In embodiments, the cancer is HER2-positive metastatic breast cancer. In embodiments, the cancer is a HER3 overexpressing cancer. In embodiments, the cancer is a HER2/HER3 overexpressing cancer. In embodiments, the cancer is a HER2 overexpressing breast cancer. In embodiments, the cancer is resistant to treatment with lapatinib. In embodiments, the cancer is resistant to treatment with vemurafenib. In embodiments, the cancer is resistant to treatment with selumetinib. In embodiments, the cancer is resistant to treatment with trastuzumab. In embodiments, the cancer is resistant to treatment with trastuzumab emtansine. In embodiments, the cancer is resistant to treatment with pertuzumab. In embodiments, the cancer is resistant to treatment with tamoxifen. In embodiments, the cancer is resistant to treatment with gefitinib. In embodiments, the cancer is resistant to treatment with erlotinib. In embodiments, the cancer is resistant to treatment with afatinib. In embodiments, the cancer is resistant to treatment with brigatinib. In embodiments, the cancer is resistant to treatment with icotinib. In embodiments, the cancer is resistant to treatment with cetuximab. In embodiments, the cancer is resistant to treatment with panitumumab. In embodiments, the cancer is resistant to treatment with zalutumumab. In embodiments, the cancer is resistant to treatment with nimotuzumab. In embodiments, the cancer is resistant to treatment with matuzumab. In embodiments, the cancer is resistant to treatment with lapatinib. In embodiments, the cancer is resistant to treatment with trastuzumab emtansine. In embodiments, a cancer that is resistant to treatment with a drug is less inhibited than after the first dose of the drug to the same cancer, less inhibited than a treatment-nave form of the cancer, less inhibited than treatment with a therapeutically effective amount of a compound described herein, less inhibited than the average cancer of the same type, or not inhibited by the drug. In embodiments, inhibition of a cancer is inhibition of cell growth. In embodiments, inhibition of a cancer is inhibition of cell proliferation.

In embodiments, the therapeutically effective amount is administered in one dose. In embodiments, the therapeutically effective amount is administered in divided doses that combine to provide a therapeutically effective amount. In embodiments, the therapeutically effective amount is administered in a single dose that is administered multiple times over the course of a treatment.

In another aspect is provided, a method of treating a disease associated with HER2 activity, wherein the method includes administering a compound described herein, or a pharmaceutically acceptable salt thereof, to a subject in need thereof.

In another aspect is provided, a method of treating a disease associated with EGFR activity, wherein the method includes administering a compound described herein, or a pharmaceutically acceptable salt thereof, to a subject in need thereof.

In embodiments, the method includes administering a therapeutically effective amount of the compound, or a pharmaceutically acceptable salt thereof, described herein.

In embodiments, the method includes administering a second agent (e.g., therapeutic agent). In embodiments, the second agent is an anti-cancer agent. In embodiments, the anti-cancer agent is an EGFR modulator, HER2 modulator, HER3 modulator, HER4 modulator, c-MET modulator, PI3K modulator, MEK modulator, MAPK modulator, RAF modulator, BRAF modulator, AKT modulator, RAS modulator, KRAS modulator, heregulin modulator, neuregulin modulator, or mTOR modulator. In embodiments, the anti-cancer agent is lapatinib, vemurafenib, or selumetinib. In embodiments, the anti-cancer agent is lapatinib, vemurafenib, or selumetinib. In embodiments, the anti-cancer agent is trastuzumab, trastuzumab emtansine, pertuzumab, tamoxifen, gefitinib, erlotinib, afatinib, brigatinib, icotinib, cetuximab, panitumumab, zalutumumab, nimotuzumab, matuzumab, or lapatinib. In embodiments, the proteins described above are human proteins.

In an aspect is provided a method of treating a disease associated with EGFR activity, HER2 activity, HER3 activity, HER4 activity, c-MET activity, PI3K activity, MEK activity, MAPK activity, RAF activity, BRAF activity, AKT activity, RAS activity, KRAS activity, heregulin activity, or neuregulin activity in a patient in need of such treatment, the method including administering a therapeutically effective amount of a compound described herein, or a pharmaceutically acceptable salt thereof. In embodiments, the proteins described above are human proteins.

In an aspect is provided a method of treating psoriasis, eczema, or atherosclerosis, in a patient in need of such treatment, the method including administering a therapeutically effective amount of a compound, or a pharmaceutically acceptable salt thereof, described herein.

In another aspect a compound described herein is provided for use as a medicament.

E. Methods of Inhibiting ERBB

In an aspect is provided a method of inhibiting an ERBB (e.g., ERBB1, ERBB2, ERBB3, or ERBB4) activity, the method including contacting ERBB (e.g., ERBB1, ERBB2, ERBB3, or ERBB4) with an effective amount of a compound described herein, or a pharmaceutically acceptable salt thereof.

In embodiments, the ERBB (e.g., ERBB1, ERBB2, ERBB3, or ERBB4) is a human ERBB (e.g., ERBB1, ERBB2, ERBB3, or ERBB4). In embodiments, ERBB (e.g., ERBB1, ERBB2, ERBB3, or ERBB4) is in an active conformation. In embodiments, ERBB (e.g., ERBB1, ERBB2, ERBB3, or ERBB4) is in an ERRB heterodimer. In embodiments the compound contacts ERBB (e.g., ERBB1, ERBB2, ERBB3, or ERBB4) and is covalently bound to ERBB (e.g., ERBB1, ERBB2, ERBB3, or ERBB4). In embodiments the compound contacts ERBB (e.g., ERBB1, ERBB2, ERBB3, or ERBB4) and is not covalently bound to ERBB (e.g., ERBB1, ERBB2, ERBB3, or ERBB4). In embodiments the compound irreversibly inhibits ERBB (e.g., ERBB1, ERBB2, ERBB3, or ERBB4). In embodiments the compound reversibly inhibits ERBB (e.g., ERBB1, ERBB2, ERBB3, or ERBB4).

F. Methods of Inhibiting HER2

In an aspect is provided a method of inhibiting HER2 activity, the method including contacting HER2 with an effective amount of a compound described herein, or a pharmaceutically acceptable salt thereof.

In embodiments, the HER2 is a human HER2. In embodiments, HER2 is in an active conformation. In embodiments, HER2 is in a HER2-HER3 heterodimer. In embodiments the compound contacts HER2 and is covalently bound to HER2. In embodiments the compound contacts HER2 and is not covalently bound to HER2. In embodiments the compound irreversibly inhibits HER2. In embodiments the compound reversibly inhibits HER2.

G. Methods of Inhibiting EGFR

In an aspect is provided a method of inhibiting EGFR activity, the method including contacting EGFR with an effective amount of a compound described herein, or a pharmaceutically acceptable salt thereof.

In embodiments, the EGFR is a human EGFR. In embodiments, EGFR is in an active conformation. In embodiments the compound contacts EGFR and is covalently bound to EGFR. In embodiments the compound contacts EGFR and is not covalently bound to EGFR. In embodiments the compound irreversibly inhibits EGFR. In embodiments the compound irreversibly inhibits EGFR. In embodiments the compound reversibly inhibits EGFR.

H. Modified Proteins

In an aspect is provided an EGFR protein covalently bonded to a compound (e.g., a compound described herein, an EGFR inhibitor). In embodiments, the compound (or a fragment thereof) is covalently bonded to a cysteine of the protein. In embodiments the EGFR includes a mutation corresponding to L858R of human EGFR. In embodiments the EGFR includes a mutation corresponding to T790M of human EGFR. In embodiments, the compound (or a fragment thereof) is covalently bonded to C797 of human EGFR. In embodiments, the compound (or a fragment thereof) is covalently bonded to a residue corresponding to C797 of human EGFR.

In embodiments, the EGFR protein covalently bonded to a compound described herein is the product of a reaction between the EGFR protein and compound described herein. It will be understood that the covalently bonded EGFR protein and compound described herein are the remnants of the reactant EGFR protein and compound, wherein each reactant now participates in the covalent bond between the EGFR protein and compound. In embodiments of the covalently bonded EGFR protein and compound described herein, the remnant of the substituted formula I is a linker including a covalent bond between the EGFR protein and the remainder of the compound described herein. It will be understood by a person of ordinary skill in the art that when a EGFR protein is covalently bonded to a compound described herein, the compound described herein forms a remnant of the pre-reacted compound wherein a bond connects the remnant of the compound to the remnant of the EGFR protein (e.g., amino acid corresponding to C797 of human EGFR). As a non-limiting example, the EGFR protein covalently bonded to a compound (e.g., a compound described herein) may have the formula:

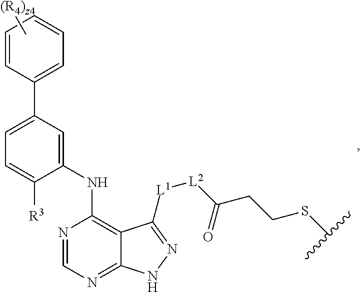

wherein S is the sulfur of a EGFR protein cysteine (e.g., corresponding to C797 of human EGFR), which is bonded to the remainder of the EGFR protein, “ [symbol] ” is the attachment point to the EGFR protein, and wherein R³, R⁴, L¹, L², and z4 are as described herein. As a non-limiting example, the EGFR protein covalently bonded to a compound (e.g., a compound described herein) may have the formula:

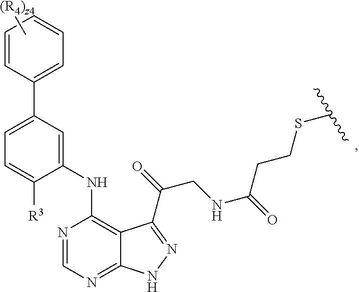

wherein S is the sulfur of a EGFR protein cysteine (e.g., corresponding to C797 of human EGFR), which is bonded to the remainder of the EGFR protein, “ [symbol] ” is the attachment point to the EGFR protein, and wherein R³, R⁴, and z4 are as described herein. As a non-limiting example, the EGFR protein covalently bonded to a compound (e.g., a compound described herein) may have the formula:

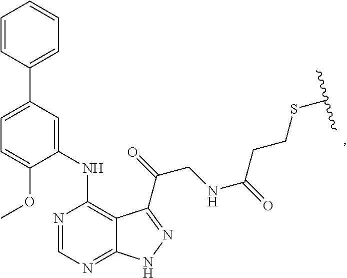

wherein S is the sulfur of a EGFR protein cysteine (e.g., corresponding to C797 of human EGFR), which is bonded to the remainder of the EGFR protein, and “ [symbol] ” is the attachment point to the EGFR protein.

In an aspect is provided a HER2 protein covalently bonded to a compound (e.g., a compound described herein, a HER2 inhibitor). In embodiments, the compound (or a fragment thereof) is covalently bonded to a cysteine of the protein. In embodiments, the compound (or a fragment thereof) is covalently bonded to C805 of human HER2. In embodiments, the compound (or a fragment thereof) is covalently bonded to a residue corresponding to C805 of human HER2.

In embodiments, the HER2 protein covalently bonded to a compound described herein is the product of a reaction between the HER2 protein and compound described herein. It will be understood that the covalently bonded HER2 protein and compound described herein are the remnants of the reactant HER2 protein and compound, wherein each reactant now participates in the covalent bond between the HER2 protein and compound. In embodiments of the covalently bonded HER2 protein and compound described herein, the remnant of the substituted formula I is a linker including a covalent bond between the HER2 protein and the remainder of the compound described herein. It will be understood by a person of ordinary skill in the art that when a HER2 protein is covalently bonded to a compound described herein, the compound forms a remnant of the pre-reacted compound wherein a bond connects the remnant of the compound to the remnant of the HER2 protein (e.g., amino acid corresponding to C805 of human HER2. As a non-limiting example, the HER2 protein covalently bonded to a compound (e.g., a compound described herein) may have the formula:

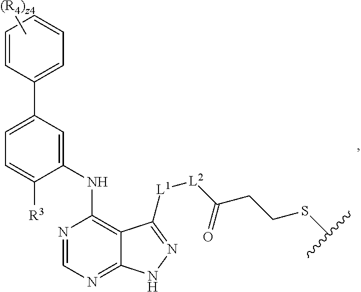

wherein S is the sulfur of a HER2 protein cysteine (e.g., corresponding to C805 of human HER2), which is bonded to the remainder of the HER2 protein, “ [symbol] ” is the attachment point to the HER2 protein, and wherein R³, R⁴, L¹, L², and z4 are as described herein. As a non-limiting example, the HER2 protein covalently bonded to a compound (e.g., a compound described herein) may have the formula:

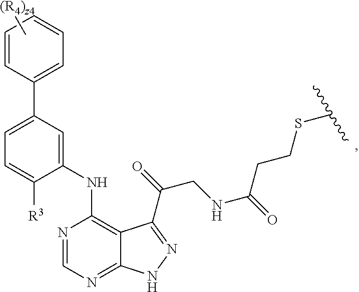

wherein S is the sulfur of a HER2 protein cysteine (e.g., corresponding to C805 of human HER2), which is bonded to the remainder of the HER2 protein, “ [symbol] ” is the attachment point to the HER2 protein, and wherein R³, R⁴, and z4 are as described herein. As a non-limiting example, the HER2 protein covalently bonded to a compound (e.g., a compound described herein) may have the formula:

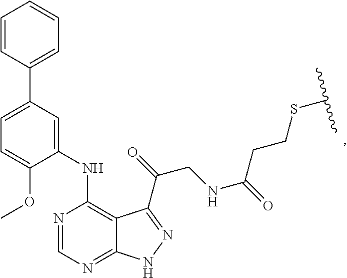

wherein S is the sulfur of a HER2 protein cysteine (e.g., corresponding to C805 of human HER2), which is bonded to the remainder of the HER2 protein and “ [symbol] ” is the attachment point to the HER2 protein.

In an aspect is provided an ERBB (e.g., ERBB1, ERBB2, ERBB3, or ERBB4) protein covalently bonded to a compound (e.g., a compound described herein, an ERBB (e.g., ERBB1, ERBB2, ERBB3, or ERBB4) inhibitor). In embodiments, the compound (or a fragment thereof) is covalently bonded to a cysteine of the protein. In embodiments, the ERBB protein covalently bonded to a compound described herein is the product of a reaction between the ERBB protein and compound described herein. It will be understood that the covalently bonded ERBB protein and compound described herein are the remnants of the reactant ERBB protein and compound, wherein each reactant now participates in the covalent bond between the ERBB protein and compound. In embodiments of the covalently bonded ERBB protein and compound described herein, the remnant of the substituted formula I is a linker including a covalent bond between the ERBB protein and the remainder of the compound described herein. It will be understood by a person of ordinary skill in the art that when a ERBB protein is covalently bonded to a compound described herein, the compound described herein forms a remnant of the pre-reacted compound wherein a bond connects the remnant of the compound to the remnant of the ERBB protein (e.g., amino acid corresponding to a cysteine amino acid of human ERBB).

### Embodiments

Embodiment P1. A compound having the formula:

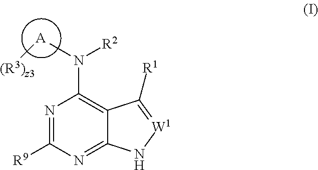

wherein Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, CHX²₂, —CH₂X², substituted or unsubstituted alkyl, or substituted or unsubstituted heteroalkyl; R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOᵥ₁₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2; wherein the compound is not

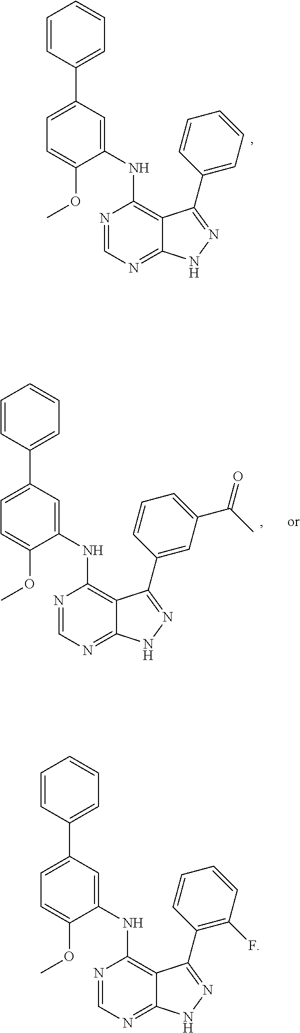 [SMILES: COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12 ; COc1ccc(-c2ccccc2)cc1Nc1ncnc2[nH]nc(-c3ccccc3F)c12]

Embodiment P2. A compound of embodiment P1, having the formula:

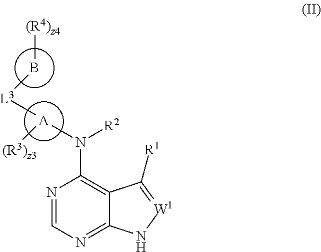

wherein Ring B is cycloalkyl, heterocycloalkyl, aryl, or heteroaryl; L³ is a bond, —S(O)₂—, —NR⁸—, —O—, —S—, —C(O)—, —C(O)NR⁸—, —NR⁸C(O)—, —NR⁸C(O)NH—, —NHC(O)NR⁸—, —C(O)O—, —OC(O)—, substituted or unsubstituted alkylene, or substituted or unsubstituted heteroalkylene; R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z4 is an integer from 0 to 5; Each R^{4A}, R^{4B}, R^{4C}, R^{4D}, R^{8A}, R^{8B}, R^{8C}, and R^{8D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl each X⁴ and X⁸ are independently —F, —Cl, —Br, or —I; n4 and n8 are independently an integer from 0 to 4; and m4, m8, v4, and v8, are independently an integer from 1 to 2.

Embodiment P3. A compound of embodiment P2, having the formula:

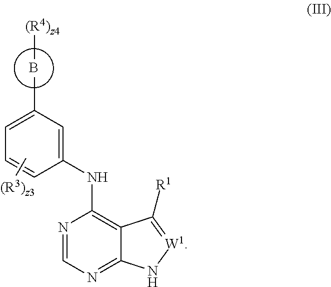

Embodiment P4. The compound of embodiment P2, having the formula:

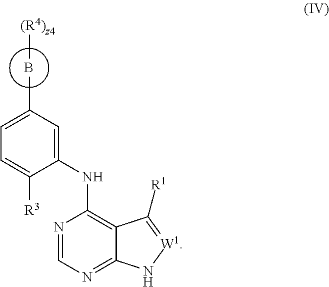

Embodiment P5. The compound of embodiment P2, having the formula:

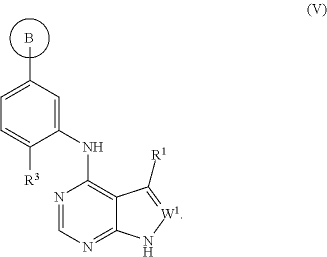

Embodiment P6. The compound of one of embodiments P1 or P5, wherein W¹ is C(H).

Embodiment P7. The compound of one of embodiments P1 or P5, wherein W¹ is N.

Embodiment P8. The compound of one of embodiments P1 or P7, wherein R³ is unsubstituted heteroalkyl.

Embodiment P9. The compound of one of embodiments P1 or P7, wherein R³ is unsubstituted 2 to 5 membered heteroalkyl.

Embodiment P10. The compound of one of embodiments P1 or P7, wherein R³ is —OCH₃, —OCH₂CH₃, —N(CH₃)₂, —NH₂, —NH(CH₃), —N(CH₂CH₃)₂, —NH(CH₂CH₃), or —SH.

Embodiment P11. The compound of one of embodiments P2 or P10, wherein Ring B is aryl or heteroaryl.

Embodiment P12. The compound of one of embodiments P2 or P10, wherein Ring B is phenyl or 5 to 6 membered heteroaryl.

Embodiment P13. The compound of one of embodiments P2 or P10, wherein Ring B is phenyl.

Embodiment P 14. The compound of one of embodiments P2 or P10, wherein Ring B is 5 to 6 membered heteroaryl.

Embodiment P15. The compound of one of embodiments P2 or P10, wherein Ring B is furanyl, thienyl, pyrrolyl, imidazolyl, pyrazolyl, oxazolyl, isoxazolyl, or thiazolyl.

Embodiment P16. The compound of one of embodiments P1 or P15, wherein R¹ is substituted or unsubstituted aryl or substituted or unsubstituted heteroaryl.

Embodiment P17. The compound of one of embodiments P1 or P15, wherein R¹ is substituted or unsubstituted phenyl or substituted or unsubstituted 5 to 6 membered heteroaryl.

Embodiment P18. The compound of one of embodiments P1 or P15, wherein R¹ is substituted or unsubstituted phenyl.

Embodiment P19. The compound of one of embodiments P1 or P15, wherein R¹ is unsubstituted phenyl.

Embodiment P20. The compound of one of embodiments P1 or P15, wherein R¹ is substituted or unsubstituted 5 to 6 membered heteroaryl.

Embodiment P21. The compound of one of embodiments P1 or P15, wherein R¹ is unsubstituted 5 to 6 membered heteroaryl.

Embodiment P22. The compound of one of embodiments P1 or P15, wherein R¹ is substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted oxazolyl, substituted or unsubstituted isoxazolyl, or substituted or unsubstituted thiazolyl.

Embodiment P23. The compound of one of embodiments P1 or P15, wherein R¹ is unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

Embodiment P24. The compound of one of embodiments P1 or P23, wherein R¹ is L¹-L²-E.

Embodiment P25. The compound of one of embodiments P1 to P24, wherein L¹ is a bond, —C(O)—, substituted or unsubstituted alkylene, or substituted or unsubstituted heteroalkylene.

Embodiment P26. The compound of one of embodiments P1 to P24, wherein L¹ is a substituted or unsubstituted C₁-C₄ alkylene.

Embodiment P27. The compound of one of embodiments P1 to P24, wherein L¹ is —C(O)CH₂CH₂CH₂—, —C(O)CH₂CH₂—, or —C(O)CH₂—.

Embodiment P28. The compound of one of embodiments P1 to P27, wherein L² is —NR⁷—, substituted or unsubstituted heteroalkylene, or substituted or unsubstituted heterocycloalkylene.

Embodiment P29. The compound of one of embodiments P1 to P27, wherein L² is —NH—.

Embodiment P30. The compound of one of embodiments P1 to P29, wherein E is a covalent cysteine modifier moiety.

Embodiment P31. The compound of one of embodiments P1 to P29, wherein E is:

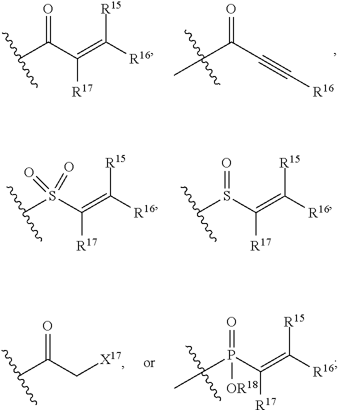

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{16D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—R^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; each X, X¹⁵, X¹⁶, X¹⁷ and X¹⁸ is independently —F, —Cl, —Br, or —I; n15, n16, n17, v15, v16, and v17, are independently an integer from 0 to 4; and

m15, m16, and m17 are independently and integer from 1 to 2.

Embodiment P32. The compound of embodiment P31, wherein R¹⁵, R¹⁶, R¹⁷, and R¹⁸ are hydrogen.

Embodiment P33. The compound of one of embodiments P31 to P32, wherein E is:

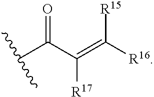

Embodiment P34. The compound of embodiment P33, wherein R¹⁵ is hydrogen; R¹⁶ is hydrogen, —CH₃, or —CH₂NR^{16A}R^{16B}, R¹⁷ is hydrogen; and R^{16A} and R^{16B} are independently hydrogen or unsubstituted alkyl.

Embodiment P35. The compound of embodiment P34, wherein R^{16A} and R^{16B} are independently unsubstituted methyl.

Embodiment P36. The compound of embodiment P33, wherein R¹⁵ is hydrogen; R¹⁶ is hydrogen; R¹⁷ is hydrogen, —CH₃, or —CH₂NR^{17A}R^{17B}, and R^{17A} and R^{17B} are independently hydrogen or unsubstituted alkyl.

Embodiment P37. The compound of embodiment P36, wherein R^{17A} and R^{17B} are independently unsubstituted methyl.

Embodiment P 38. The compound of embodiment P33, wherein R¹⁵ is hydrogen, —CH₃, or —CH₂NR^{15A}R^{15B}; R¹⁶ is hydrogen; R¹⁷ is hydrogen; and R^{15A} and R^{15B} are independently hydrogen or unsubstituted alkyl.

Embodiment P39. The compound of embodiment P38, wherein R^{15A} and R^{15B} are independently unsubstituted methyl.

Embodiment P40. The compound of embodiment Pl,wherein the compound has the formula:

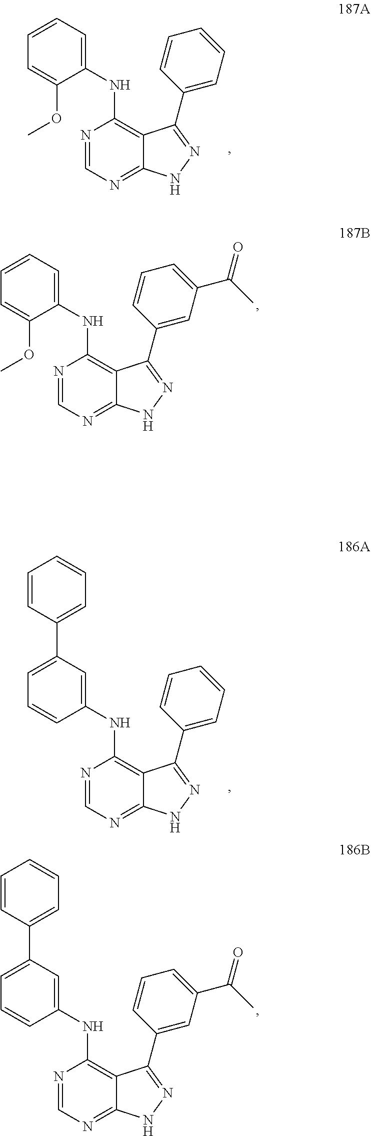 [SMILES: COc1ccccc1Nc1ncnc2[nH]nc(-c3ccccc3)c12 ; COc1ccccc1Nc1ncnc2[nH]nc(-c3cccc(C(C)=O)c3)c12 ; c1ccc(-c2cccc(Nc3ncnc4[nH]nc(-c5ccccc5)c34)c2)cc1 ; CC(=O)c1cccc(-c2n[nH]c3ncnc(Nc4cccc(-c5ccccc5)c4)c23)c1]

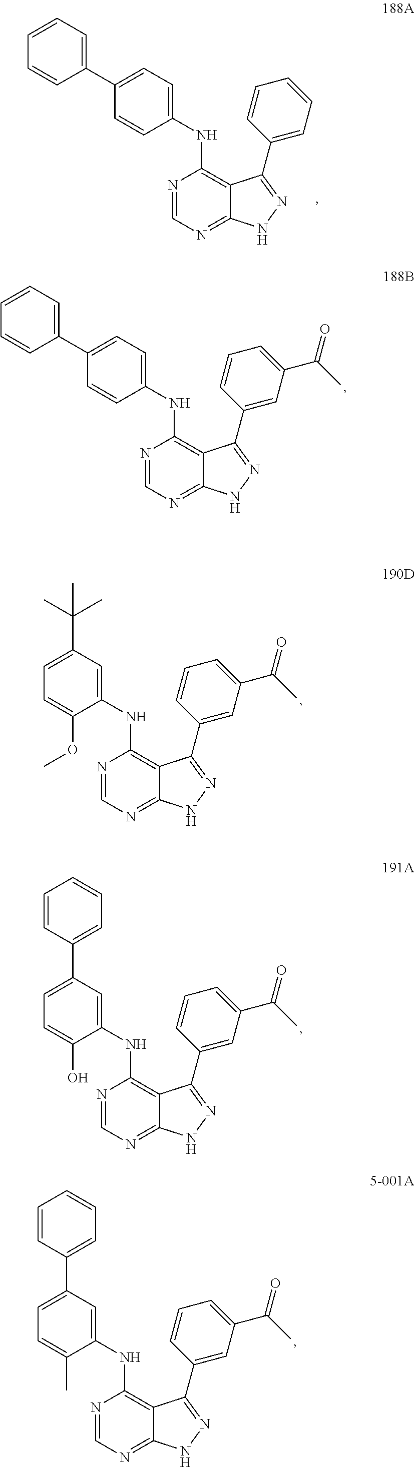 [SMILES: c1ccc(-c2ccc(Nc3ncnc4[nH]nc(-c5ccccc5)c34)cc2)cc1 ; CC(=O)c1cccc(-c2n[nH]c3ncnc(Nc4ccc(-c5ccccc5)cc4)c23)c1 ; COc1ccc(C(C)(C)C)cc1Nc1ncnc2[nH]nc(-c3cccc(C(C)=O)c3)c12 ; CC(=O)c1cccc(-c2n[nH]c3ncnc(Nc4cc(-c5ccccc5)ccc4O)c23)c1 ; CC(=O)c1cccc(-c2n[nH]c3ncnc(Nc4cc(-c5ccccc5)ccc4C)c23)c1]

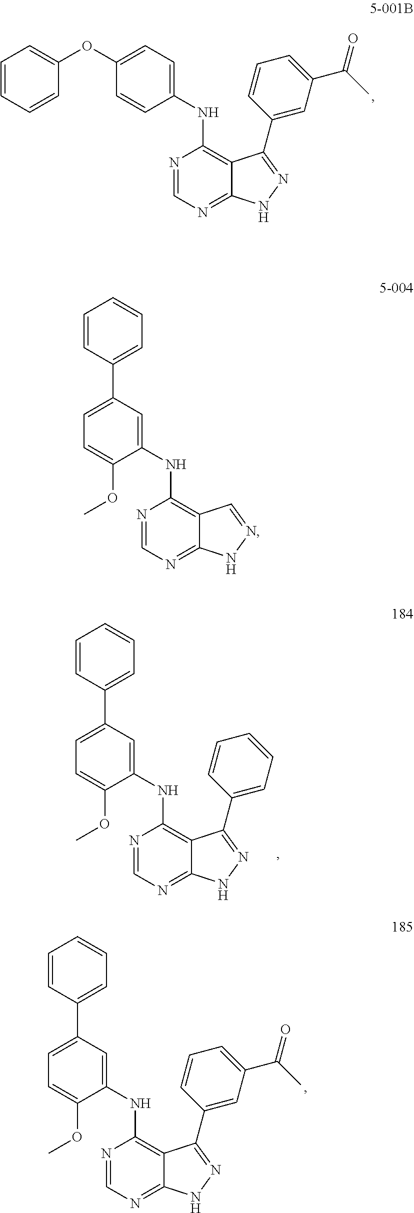 [SMILES: CC(=O)c1cccc(-c2n[nH]c3ncnc(Nc4ccc(Oc5ccccc5)cc4)c23)c1 ; COc1ccc(-c2ccccc2)cc1Nc1ncnc2[nH]ncc12 ; COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12]

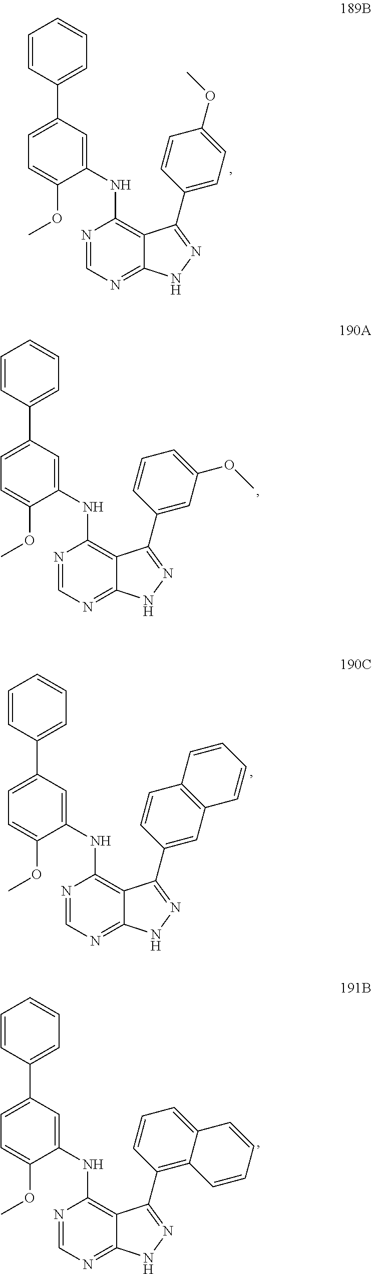 [SMILES: COc1ccc(-c2n[nH]c3ncnc(Nc4cc(-c5ccccc5)ccc4OC)c23)cc1 ; COc1cccc(-c2n[nH]c3ncnc(Nc4cc(-c5ccccc5)ccc4OC)c23)c1 ; COc1ccc(-c2ccccc2)cc1Nc1ncnc2[nH]nc(-c3ccc4ccccc4c3)c12 ; COc1ccc(-c2ccccc2)cc1Nc1ncnc2[nH]nc(-c3cccc4ccccc34)c12]

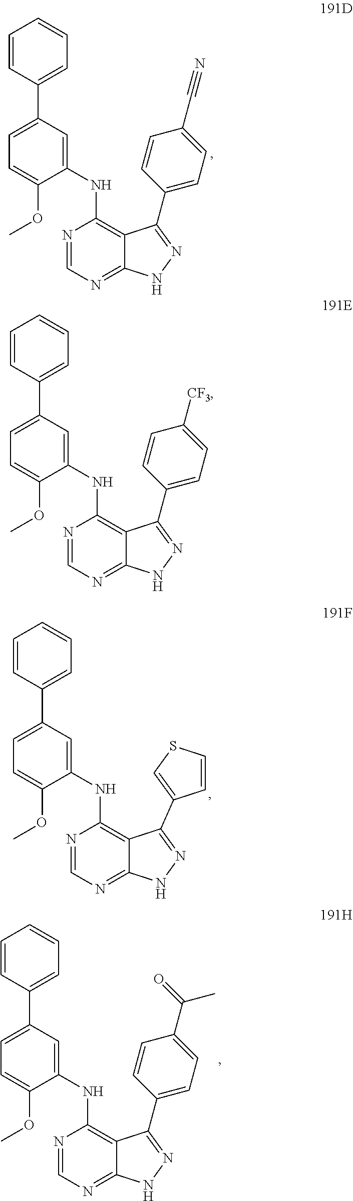 [SMILES: COc1ccc(-c2ccccc2)cc1Nc1ncnc2[nH]nc(-c3ccc(C#N)cc3)c12 ; COc1ccc(-c2ccccc2)cc1Nc1ncnc2[nH]nc(-c3ccc(C(F)(F)F)cc3)c12 ; COc1ccc(-c2ccccc2)cc1Nc1ncnc2[nH]nc(-c3ccsc3)c12 ; COc1ccc(-c2ccccc2)cc1Nc1ncnc2[nH]nc(-c3ccc(C(C)=O)cc3)c12]

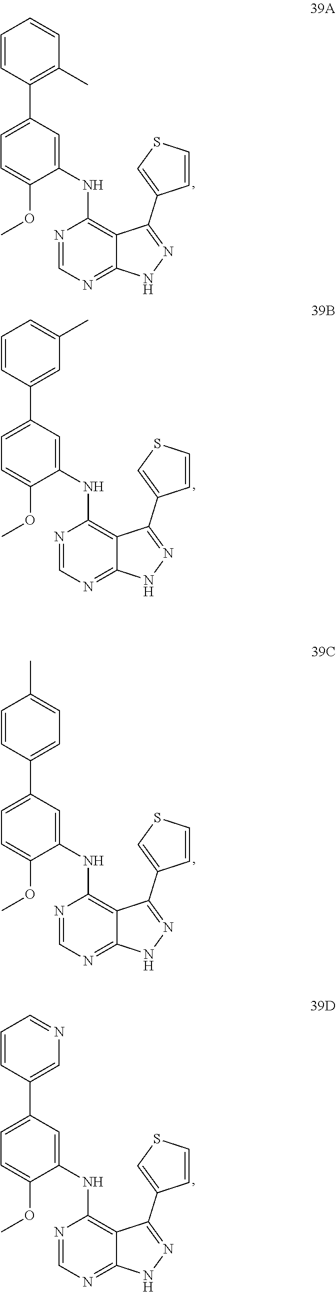

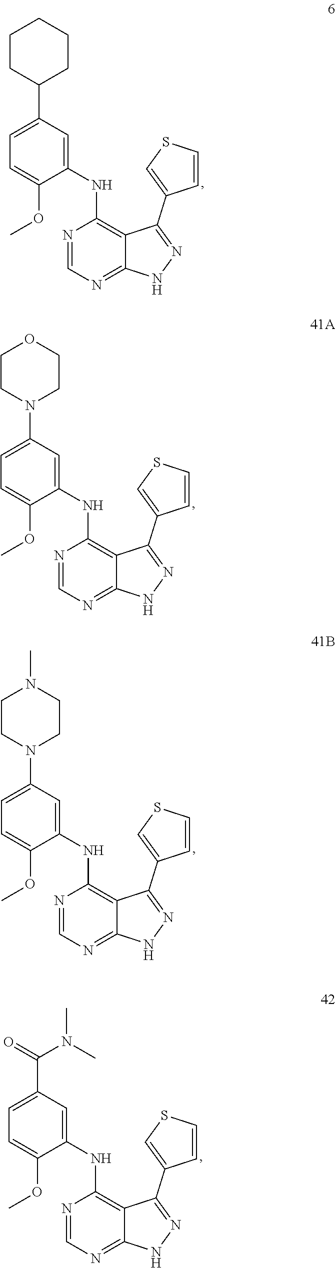 [SMILES: COc1ccc(C2CCCCC2)cc1Nc1ncnc2[nH]nc(-c3ccsc3)c12 ; COc1ccc(N2CCOCC2)cc1Nc1ncnc2[nH]nc(-c3ccsc3)c12 ; COc1ccc(N2CCN(C)CC2)cc1Nc1ncnc2[nH]nc(-c3ccsc3)c12 ; COc1ccc(C(=O)N(C)C)cc1Nc1ncnc2[nH]nc(-c3ccsc3)c12]

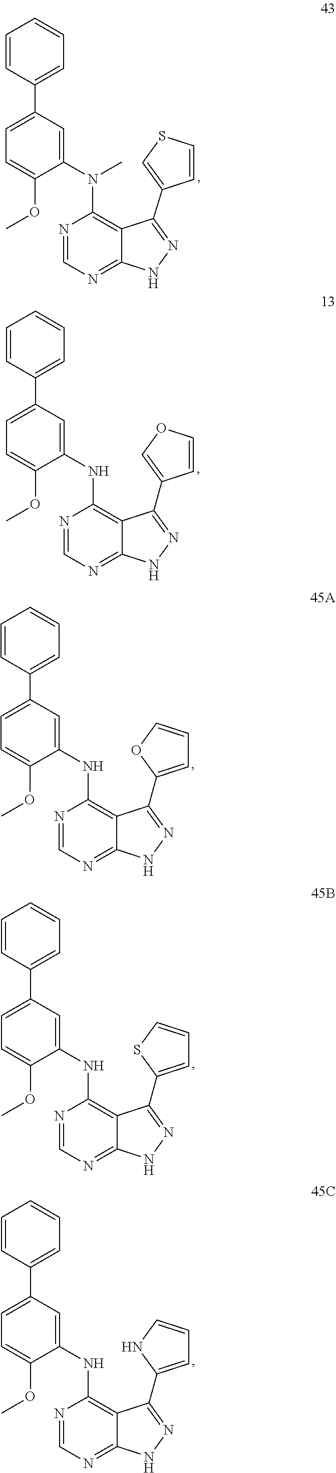 [SMILES: COc1ccc(-c2ccccc2)cc1N(C)c1ncnc2[nH]nc(-c3ccsc3)c12 ; COc1ccc(-c2ccccc2)cc1Nc1ncnc2[nH]nc(-c3ccoc3)c12 ; COc1ccc(-c2ccccc2)cc1Nc1ncnc2[nH]nc(-c3ccco3)c12 ; COc1ccc(-c2ccccc2)cc1Nc1ncnc2[nH]nc(-c3cccs3)c12 ; COc1ccc(-c2ccccc2)cc1Nc1ncnc2[nH]nc(-c3ccc[nH]3)c12]

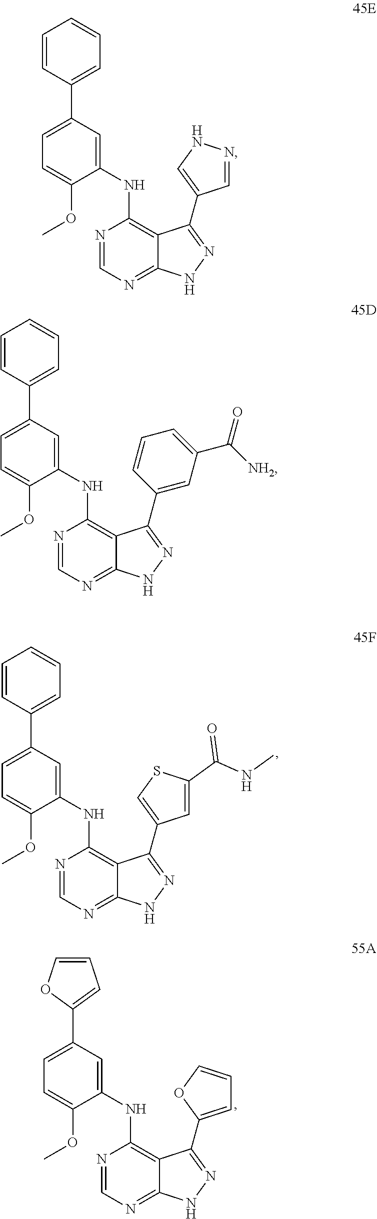

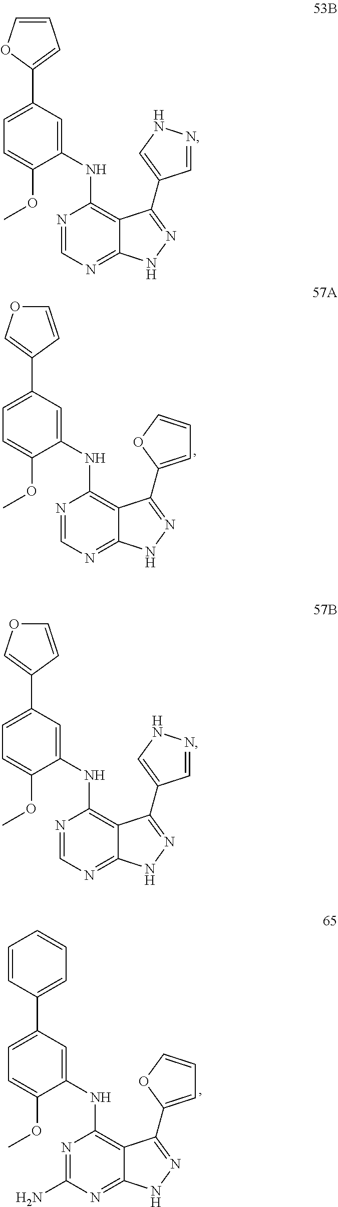 [SMILES: COc1ccc(-c2ccco2)cc1Nc1ncnc2[nH]nc(-c3cn[nH]c3)c12 ; COc1ccc(-c2ccoc2)cc1Nc1ncnc2[nH]nc(-c3ccco3)c12 ; COc1ccc(-c2ccoc2)cc1Nc1ncnc2[nH]nc(-c3cn[nH]c3)c12 ; COc1ccc(-c2ccccc2)cc1Nc1nc(N)nc2[nH]nc(-c3ccco3)c12]

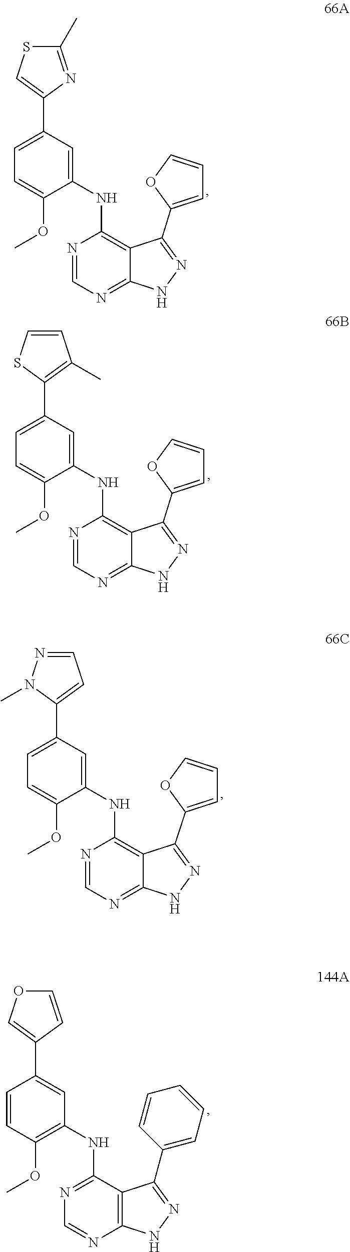 [SMILES: COc1ccc(-c2csc(C)n2)cc1Nc1ncnc2[nH]nc(-c3ccco3)c12 ; COc1ccc(-c2sccc2C)cc1Nc1ncnc2[nH]nc(-c3ccco3)c12 ; COc1ccc(-c2ccnn2C)cc1Nc1ncnc2[nH]nc(-c3ccco3)c12 ; COc1ccc(-c2ccoc2)cc1Nc1ncnc2[nH]nc(-c3ccccc3)c12]

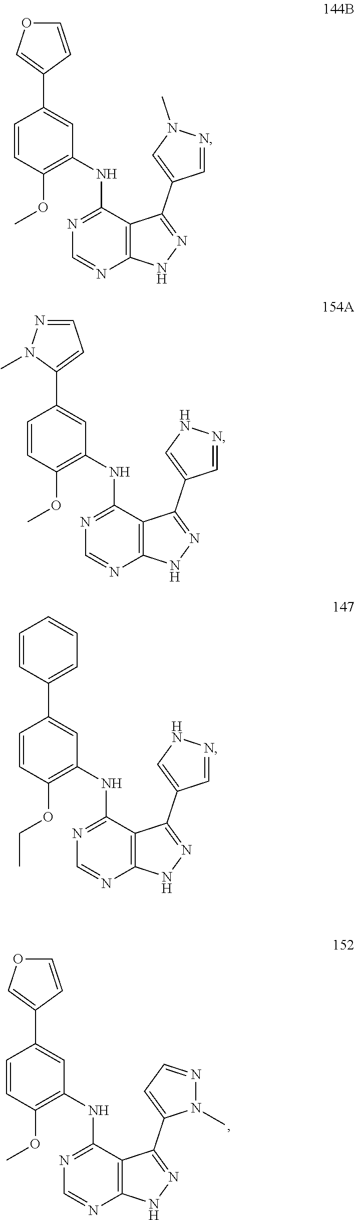 [SMILES: COc1ccc(-c2ccoc2)cc1Nc1ncnc2[nH]nc(-c3cnn(C)c3)c12 ; COc1ccc(-c2ccnn2C)cc1Nc1ncnc2[nH]nc(-c3cn[nH]c3)c12 ; CCOc1ccc(-c2ccccc2)cc1Nc1ncnc2[nH]nc(-c3cn[nH]c3)c12 ; COc1ccc(-c2ccoc2)cc1Nc1ncnc2[nH]nc(-c3ccnn3C)c12]

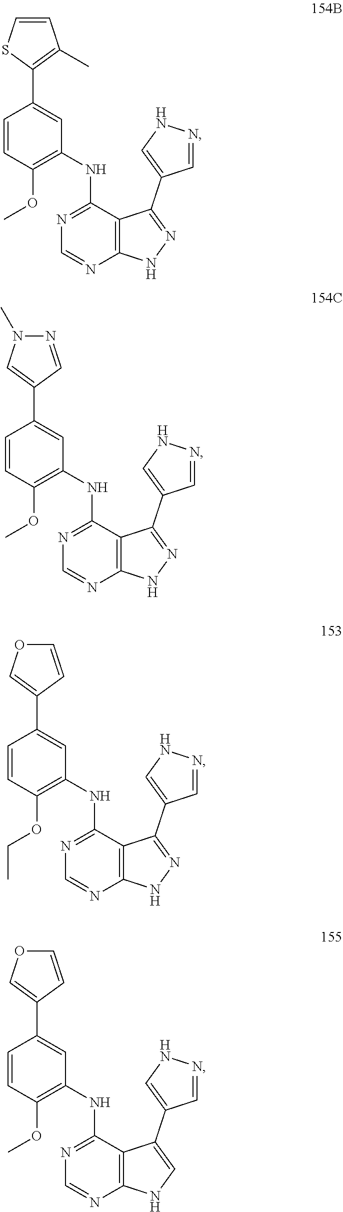 [SMILES: COc1ccc(-c2sccc2C)cc1Nc1ncnc2[nH]nc(-c3cn[nH]c3)c12 ; COc1ccc(-c2cnn(C)c2)cc1Nc1ncnc2[nH]nc(-c3cn[nH]c3)c12 ; CCOc1ccc(-c2ccoc2)cc1Nc1ncnc2[nH]nc(-c3cn[nH]c3)c12 ; COc1ccc(-c2ccoc2)cc1Nc1ncnc2[nH]cc(-c3cn[nH]c3)c12]

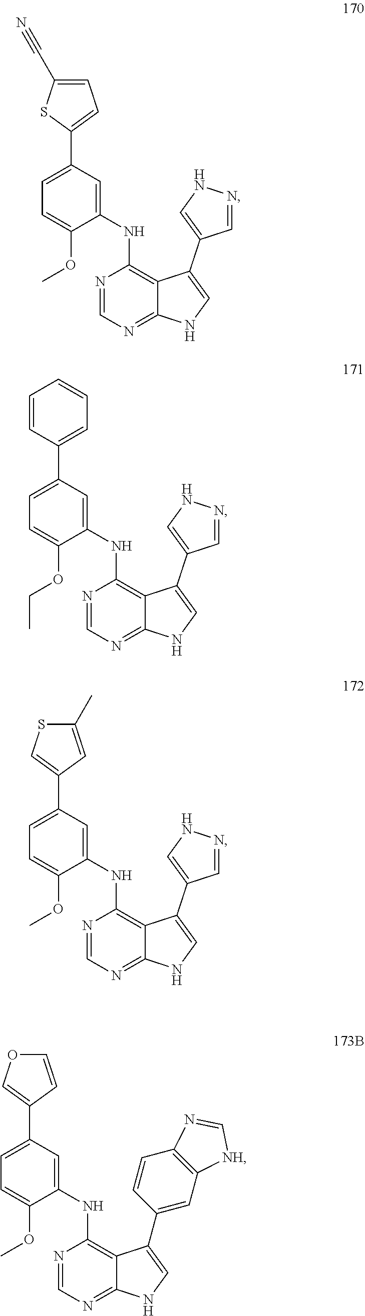 [SMILES: COc1ccc(-c2ccc(C#N)s2)cc1Nc1ncnc2[nH]cc(-c3cn[nH]c3)c12 ; CCOc1ccc(-c2ccccc2)cc1Nc1ncnc2[nH]cc(-c3cn[nH]c3)c12 ; COc1ccc(-c2csc(C)c2)cc1Nc1ncnc2[nH]cc(-c3cn[nH]c3)c12 ; COc1ccc(-c2ccoc2)cc1Nc1ncnc2[nH]cc(-c3ccc4nc[nH]c4c3)c12]

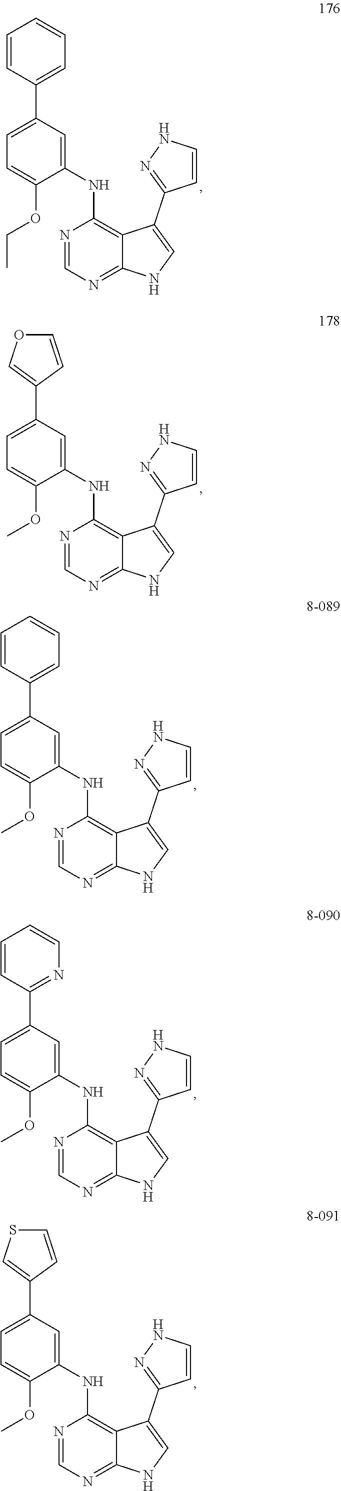 [SMILES: CCOc1ccc(-c2ccccc2)cc1Nc1ncnc2[nH]cc(-c3cc[nH]n3)c12 ; COc1ccc(-c2ccoc2)cc1Nc1ncnc2[nH]cc(-c3cc[nH]n3)c12 ; COc1ccc(-c2ccccc2)cc1Nc1ncnc2[nH]cc(-c3cc[nH]n3)c12 ; COc1ccc(-c2ccccn2)cc1Nc1ncnc2[nH]cc(-c3cc[nH]n3)c12 ; COc1ccc(-c2ccsc2)cc1Nc1ncnc2[nH]cc(-c3cc[nH]n3)c12]

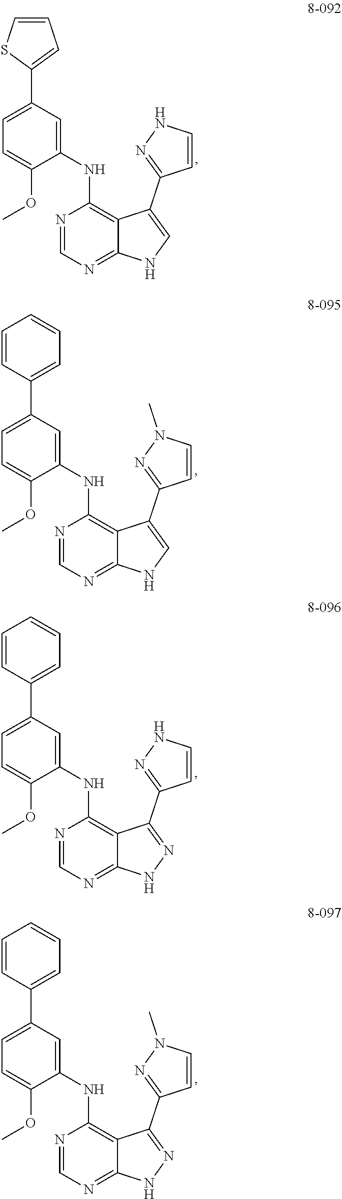 [SMILES: COc1ccc(-c2cccs2)cc1Nc1ncnc2[nH]cc(-c3cc[nH]n3)c12 ; COc1ccc(-c2ccccc2)cc1Nc1ncnc2[nH]cc(-c3ccn(C)n3)c12 ; COc1ccc(-c2ccccc2)cc1Nc1ncnc2[nH]nc(-c3cc[nH]n3)c12 ; COc1ccc(-c2ccccc2)cc1Nc1ncnc2[nH]nc(-c3ccn(C)n3)c12]

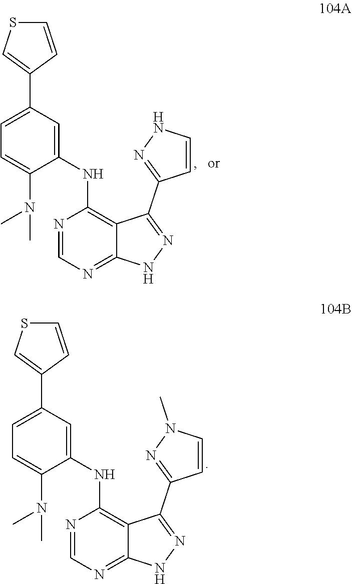 [SMILES: CN(C)c1ccc(-c2ccsc2)cc1Nc1ncnc2[nH]nc(-c3cc[nH]n3)c12 ; CN(C)c1ccc(-c2ccsc2)cc1Nc1ncnc2[nH]nc(-c3ccn(C)n3)c12]

Embodiment P41. A pharmaceutical composition comprising a compound of one of embodiments P1 to P39 or a pharmaceutically acceptable salt thereof, and a pharmaceutically acceptable excipient.

Embodiment P42. The pharmaceutical composition of embodiment P41, further comprising an anti-cancer agent.

Embodiment P43. A method of treating a disease associated with HER2 activity in a patient in need of such treatment, said method comprising administering a therapeutically effective amount of a compound, or a pharmaceutically acceptable salt thereof, having the formula:

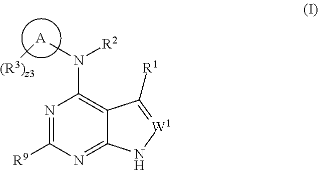

wherein Ring A is aryl or heteroaryl; W¹ is N or C(H); R¹ is hydrogen, L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment P44. A method of treating a disease associated with EGFR activity in a patient in need of such treatment, said method comprising administering a therapeutically effective amount of a compound, or a pharmaceutically acceptable salt thereof, having the formula:

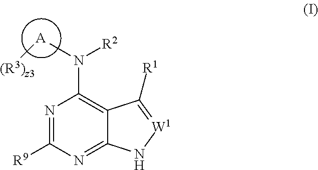

wherein Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment P45. A method of treating cancer in a patient in need of such treatment, said method comprising administering a therapeutically effective amount of a compound, or a pharmaceutically acceptable salt thereof, having the formula:

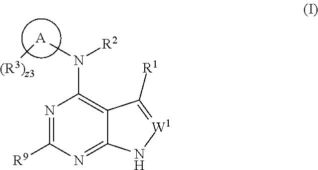

wherein Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment P46. The method of embodiment P45, wherein the cancer is resistant to a HER2 inhibitor.

Embodiment P47. The method of embodiment P45, wherein the cancer is resistant to an EGFR inhibitor.

Embodiment P48. A method of inhibiting HER2 activity, said method comprising contacting HER2 with an effective amount of a compound, or a pharmaceutically acceptable salt thereof, having the formula:

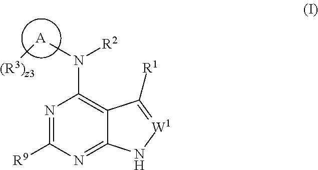

wherein Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment P49. The method of embodiment P48, wherein HER2 is in an active conformation.

Embodiment P50. The method of embodiment P49, wherein HER2 is in a HER2-HER3 heterodimer.

Embodiment P51. A method of inhibiting EGFR activity, said method comprising contacting EGFR with an effective amount of a compound, or a pharmaceutically acceptable salt thereof, having the formula:

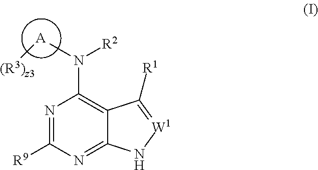

wherein Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —O—, —S—, —C(O)—, —C(O) NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment P52. The method of embodiment P51, wherein EGFR is in an active conformation.

Embodiment P53. A method of one of embodiments P43 to P52, wherein the compound has the formula:

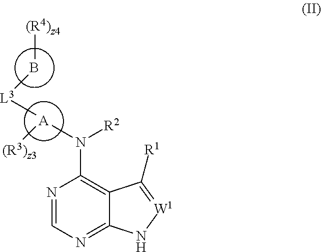

wherein Ring B is cycloalkyl, heterocycloalkyl, aryl, or heteroaryl; L³ is a bond, —S(O)₂—, —NR⁸—, —O—, —S—, —C(O)—, —C(O)NR⁸—, —NR⁸C(O)—, —NR⁸C(O)NH—, —NHC(O)NR⁸—, —C(O)O—, —OC(O)—, substituted or unsubstituted alkylene, or substituted or unsubstituted heteroalkylene; R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z4 is an integer from 0 to 5; Each R^{4A}, R^{4B}, R^{4C}, R^{4D}, R^{8A}, R^{8B}, R^{8C}, and R^{8D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl each X⁴ and X⁸ are independently —F, —Cl, —Br, or —I; n4 and n8 are independently an integer from 0 to 4; and m4, m8, v4, and v8, are independently an integer from 1 to 2.

Embodiment P54. A method of one of embodiments P43 to P52, wherein the compound has the formula:

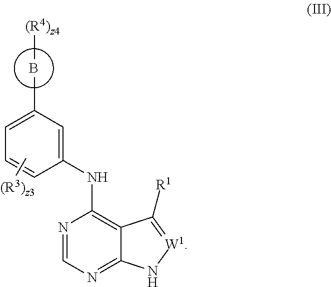

Embodiment P55. A method of one of embodiments P43 to P52, wherein the compound has the formula:

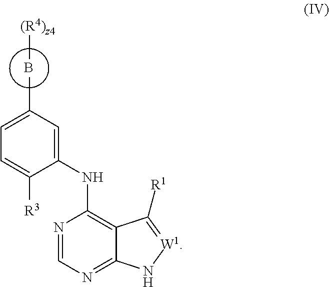

Embodiment P56. A method of one of embodiments P43 to P52, wherein the compound has the formula:

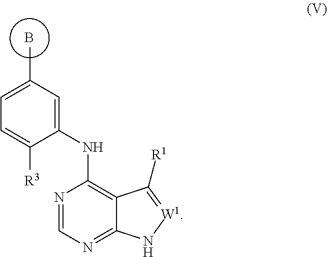

Embodiment P57. The method of one of embodiments P43 to P56, wherein W¹ is C(H).

Embodiment P58. The method of one of embodiments P43 to P56, wherein W¹ is N.

Embodiment P59. The method of one of embodiments P43 to P58, wherein R³ is unsubstituted heteroalkyl.

Embodiment P60. The method of one of embodiments P43 to P58, wherein R³ is unsubstituted 2 to 5 membered heteroalkyl.

Embodiment P61. The method of one of embodiments P43 to P58, wherein R³ is —OCH₃, —OCH₂CH₃, —N(CH₃)₂, —NH₂, —NH(CH₃), —N(CH₂CH₃)₂, —NH(CH₂CH₃), or —SH.

Embodiment P62. The method of one of embodiments P44 to P61, wherein Ring B is aryl or heteroaryl.

Embodiment P63. The method of one of embodiments P44 to P61, wherein Ring B is phenyl or 5 to 6 membered heteroaryl.

Embodiment P64. The method of one of embodiments P44 to P61, wherein Ring B is phenyl.

Embodiment P65. The method of one of embodiments P44 to P61, wherein Ring B is 5 to 6 membered heteroaryl.

Embodiment P66. The method of one of embodiments P44 to P61, wherein Ring B is furanyl, thienyl, pyrrolyl, imidazolyl, pyrazolyl, oxazolyl, isoxazolyl, or thiazolyl.

Embodiment P67. The method of one of embodiments P43 to P66, wherein R¹ is substituted or unsubstituted aryl or substituted or unsubstituted heteroaryl.

Embodiment P68. The method of one of embodiments P43 to P66, wherein R¹ is substituted or unsubstituted phenyl or substituted or unsubstituted 5 to 6 membered heteroaryl.

Embodiment P69. The method of one of embodiments P43 to P66, wherein R¹ is substituted or unsubstituted phenyl.

Embodiment P70. The method of one of embodiments P43 to P66, wherein R¹ is unsubstituted phenyl.

Embodiment P71. The method of one of embodiments P43 to P66, wherein R¹ is substituted or unsubstituted 5 to 6 membered heteroaryl.

Embodiment P72. The method of one of embodiments P43 to P66, wherein R¹ is unsubstituted 5 to 6 membered heteroaryl.

Embodiment P73. The method of one of embodiments P43 to P66, wherein R¹ is substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted oxazolyl, substituted or unsubstituted isoxazolyl, or substituted or unsubstituted thiazolyl.

Embodiment P74. The method of one of embodiments P43 to P66, wherein R¹ is unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

Embodiment P75. The method of one of embodiments P43 to P74, wherein R¹ is —L¹-L²-E.

Embodiment P76. The method of one of embodiments P43 to P75, wherein L¹ is a bond, —C(O)—, substituted or unsubstituted alkylene, or substituted or unsubstituted heteroalkylene.

Embodiment P77. The method of one of embodiments P43 to P75, wherein L¹ is a substituted or unsubstituted C₁-C₄ alkylene.

Embodiment P78. The method of one of embodiments P43 to P75, wherein L¹ is —C(O)CH₂CH₂CH₂—, —C(O)CH₂CH₂—, or —C(O)CH₂—.

Embodiment P79. The method of one of embodiments P43 to P78, wherein L² is —NR⁷—, substituted or unsubstituted heteroalkylene, or substituted or unsubstituted heterocycloalkylene.

Embodiment P80. The method of one of embodiments P43 to P78, wherein L² is —NH—.

Embodiment P81. The method of one of embodiments P43 to P80, wherein E is a covalent cysteine modifier moiety.

Embodiment P82. The method of one of embodiments P43 to P80, wherein E is:

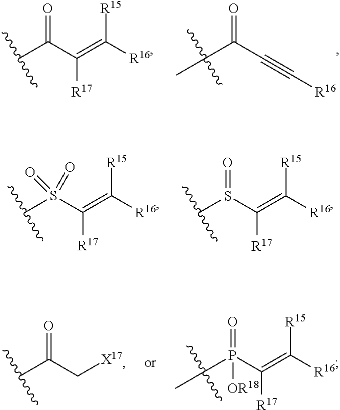

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; each X, X¹⁵, X¹⁶, X¹⁷ and X¹⁸ is independently —F, —Cl, —Br, or —I; n15, n16, n17, v15, v16, and v17, are independently an integer from 0 to 4; and m15, m16, and m17 are independently and integer from 1 to 2.

Embodiment P83. The method of embodiment P82, wherein R¹⁵, R¹⁶, R¹⁷, and R¹⁸ are hydrogen.

Embodiment P84. The method of one of embodiments P82 to P83, wherein E is:

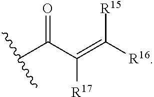

Embodiment P85. The method of embodiment P84, wherein R¹⁵ is hydrogen; R¹⁶ is hydrogen, —CH₃, or —CH₂NR^{16A}R^{16B}, R¹⁷ is hydrogen; and R^{16A} and R^{16B} are independently hydrogen or unsubstituted alkyl.

Embodiment P86. The method of embodiment P85, wherein R^{16A} and R^{16B} are independently unsubstituted methyl.

Embodiment P87. The method of embodiment P84, wherein R¹⁵ is hydrogen; R¹⁶ is hydrogen; R¹⁷ is hydrogen, —CH₃, or —CH₂NR^{17A}R^{17B}, and R^{17A} and R^{17B} are independently hydrogen or unsubstituted alkyl.

Embodiment P88. The method of embodiment P87, wherein R^{17A} and R^{17B} are independently unsubstituted methyl.

Embodiment P89. The compound of embodiment P84, wherein R¹⁵ is hydrogen, —CH₃, or —CH₂NR^{15A}R^{15B}, R¹⁶ is hydrogen; R¹⁷ is hydrogen; and R^{15A} and R^{15B} are independently hydrogen or unsubstituted alkyl.

Embodiment P90. The method of embodiment P89, wherein R^{15A} and R^{15B} are independently unsubstituted methyl.

Embodiment P91. An EGFR protein covalently bonded to a compound having the formula:

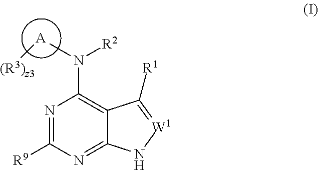

wherein Ring A is aryl or heteroaryl; W¹ is N or C(H); R¹ is -L¹-L²-E; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment P92. A HER2 protein covalently bonded to a compound having the formula:

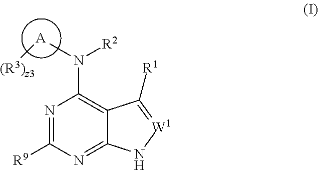

wherein Ring A is aryl or heteroaryl; W¹ is N or C(H); R¹ is -L¹-L²-E; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; Each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

### Additional Embodiments

Embodiment 1. A compound having the formula:

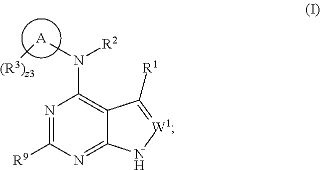

wherein Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2; wherein the compound is not

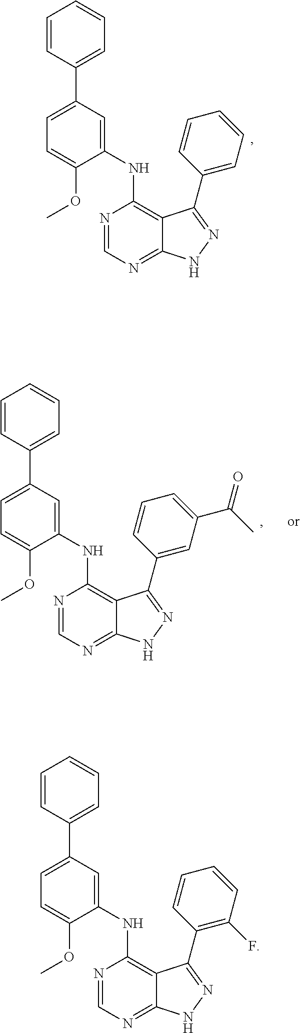 [SMILES: COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12 ; COc1ccc(-c2ccccc2)cc1Nc1ncnc2[nH]nc(-c3ccccc3F)c12]

Embodiment 2. A compound of embodiment 1, having the formula:

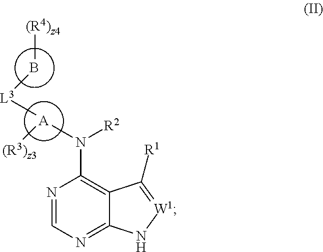

wherein Ring B is cycloalkyl, heterocycloalkyl, aryl, or heteroaryl; L³ is a bond, —S(O)₂—, —NR⁸—, —O—, —S—, —C(O)—, —C(O)NR⁸—, —NR⁸C(O)—, —NR⁸C(O)NH—, —NHC(O)NR⁸—, —C(O)O—, —OC(O)—, substituted or unsubstituted alkylene, or substituted or unsubstituted heteroalkylene; R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹ is independently halogen, —CX⁸₃, —CHX⁸₂, —CH₂X⁸, —OCX⁸₃, —OCH₂X⁸, —OCHX⁸₂, —CN, —SOₙ₈R^{D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z4 is an integer from 0 to 5; each R^{4A}, R^{4B}, R^{4C}, R^{4D}, R^{8A}, R^{8B}, R^{8C}, and R^{8D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; each X⁴ and X⁸ are independently —F, —Cl, —Br, or —I; n4 and n8 are independently an integer from 0 to 4; and m4, m8, v4, and v8, are independently an integer from 1 to 2.

Embodiment 3. A compound of embodiment 2, having the formula:

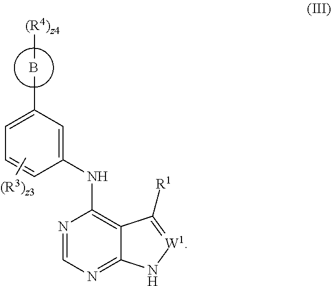

Embodiment 4. The compound of embodiment 2, having the formula:

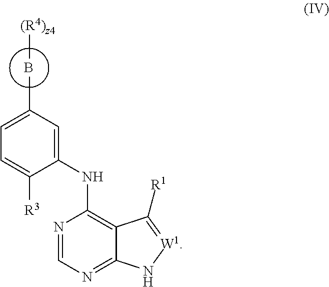

Embodiment 5. The compound of one of embodiments 2 to 4, wherein R⁴ is independently halogen, —CX⁴₃, —CHX⁴₂, —CH₂X⁴, —OCX⁴₃, —OCH₂X⁴, —OCHX⁴₂, or —CN.

Embodiment 6. The compound of one of embodiments 2 to 4, wherein R⁴ is independently halogen, —CX⁴₃, —CHX⁴₂, or —CH₂X⁴.

Embodiment 7. The compound of one of embodiments 2 to 4, wherein R⁴ is independently halogen.

Embodiment 8. The compound of embodiment 2, having the formula:

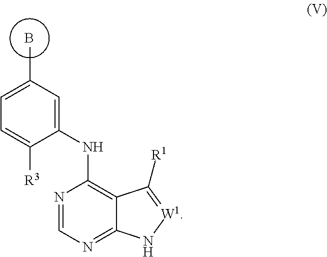

Embodiment 9. The compound of one of embodiments 1 to 8, wherein W¹ is C(H).

Embodiment 10. The compound of one of embodiments 1 to 8, wherein W¹ is N.

Embodiment 11. The compound of one of embodiments 1 to 10, wherein R³ is an unsubstituted heteroalkyl.

Embodiment 12. The compound of one of embodiments 1 to 10, wherein R³ is an unsubstituted 2 to 5 membered heteroalkyl.

Embodiment 13. The compound of one of embodiments 1 to 10, wherein R³ is —OCH₃, —OCH₂CH₃, —N(CH₃)₂, —NH₂, —NH(CH₃), —N(CH₂CH₃)₂, —NH(CH₂CH₃), —OCX³₃, —OCH₂X³, —OCHX³₂, or —SH.

Embodiment 14. The compound of one of embodiments 1 to 10, wherein R³ is —OCH₃, —OCH₂CH₃, —N(CH₃)₂, —OCX³₃, —OCH₂X³, or —OCHX³₂.

Embodiment 15. The compound of one of embodiments 2 to 13, wherein Ring B is substituted or unsubstituted aryl or heteroaryl.

Embodiment 16. The compound of one of embodiments 2 to 13, wherein Ring B is substituted or unsubstituted phenyl or 5 to 6 membered heteroaryl.

Embodiment 17. The compound of one of embodiments 2 to 13, wherein Ring B is substituted or unsubstituted phenyl.

Embodiment 18. The compound of one of embodiments 2 to 13, wherein Ring B is substituted or unsubstituted 5 to 6 membered heteroaryl.

Embodiment 19. The compound of one of embodiments 2 to 13, wherein Ring B is substituted or unsubstituted pyrazinyl, pyrimidinyl, furanyl, thienyl, pyrrolyl, imidazolyl, pyrazolyl, oxazolyl, isoxazolyl, or thiazolyl.

Embodiment 20. The compound of one of embodiments 1 to 19, wherein R¹ is substituted or unsubstituted aryl or substituted or unsubstituted heteroaryl.

Embodiment 21. The compound of one of embodiments 1 to 19, wherein R¹ is substituted or unsubstituted phenyl or substituted or unsubstituted 5 to 6 membered heteroaryl.

Embodiment 22. The compound of one of embodiments 1 to 19, wherein R¹ is substituted or unsubstituted phenyl.

Embodiment 23. The compound of one of embodiments 1 to 19, wherein R¹ is an unsubstituted phenyl.

Embodiment 24. The compound of one of embodiments 1 to 19, wherein R¹ is substituted or unsubstituted 5 to 6 membered heteroaryl.

Embodiment 25. The compound of one of embodiments 1 to 19, wherein R¹ is an unsubstituted 5 to 6 membered heteroaryl.

Embodiment 26. The compound of one of embodiments 1 to 19, wherein R¹ is substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted oxazolyl, substituted or unsubstituted isoxazolyl, or substituted or unsubstituted thiazolyl.

Embodiment 27. The compound of one of embodiments 1 to 19, wherein R¹ is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

Embodiment 28. The compound of one of embodiments 1 to 27, wherein R¹ is -L¹-L²-E.

Embodiment 29. The compound of one of embodiments 1 to 28, wherein L¹ is a bond, —C(O)—, substituted or unsubstituted alkylene, or substituted or unsubstituted heteroalkylene.

Embodiment 30. The compound of one of embodiments 1 to 28, wherein L¹ is a substituted or unsubstituted C₁-C₄ alkylene.

Embodiment 31. The compound of one of embodiments 1 to 28, wherein L¹ is —C(O)CH₂CH₂CH₂—, —C(O)CH₂CH₂—, or —C(O)CH₂—.

Embodiment 32. The compound of one of embodiments 1 to 31, wherein L² is —NR⁷—, substituted or unsubstituted heteroalkylene, or substituted or unsubstituted heterocycloalkylene.

Embodiment 33. The compound of one of embodiments 1 to 31, wherein L² is —NH—.

Embodiment 34. The compound of one of embodiments 1 to 33, wherein E is a covalent cysteine modifier moiety.

Embodiment 35. The compound of one of embodiments 1 to 33, wherein E is:

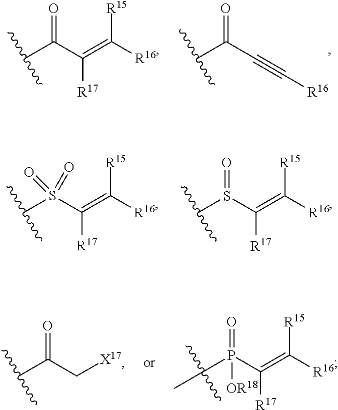

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; each X, X¹⁵, X¹⁶, X¹⁷ and X¹⁸ is independently —F, —Cl, —Br, or —I; n15, n16, n17, v15, v16, and v17, are independently an integer from 0 to 4; and m15, m16, and m17 are independently and integer from 1 to 2.

Embodiment 36. The compound of embodiment 35, wherein R¹⁵, R¹⁶, R¹⁷, and R¹⁸ are hydrogen.

Embodiment 37. The compound of one of embodiments 35 to 36, wherein E is:

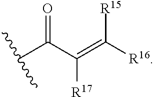

Embodiment 38. The compound of embodiment 37, wherein R¹⁵ is hydrogen; R¹⁶ is hydrogen, —CH₃, or —CH₂NR^{16A}R^{16B}, R¹⁷ is hydrogen; and R^{16A} and R^{16B} are independently hydrogen or unsubstituted alkyl.

Embodiment 39. The compound of embodiment 38, wherein R^{16A} and R^{16B} are independently unsubstituted methyl.

Embodiment 40. The compound of embodiment 37, wherein R¹⁵ is hydrogen; R¹⁶ is hydrogen; R¹⁷ is hydrogen, —CH₃, or —CH₂NR^{17A}R^{17B}, and R^{17A} and R^{17B} are independently hydrogen or unsubstituted alkyl.

Embodiment 41. The compound of embodiment 40, wherein R^{17A} and R^{17B} are independently unsubstituted methyl.

Embodiment 42. The compound of embodiment 37, wherein R¹⁵ is hydrogen, —CH₃, or —CH₂NR^{15A}R^{15B}; R¹⁶ is hydrogen; R¹⁷ is hydrogen; and R^{15A} and R^{15B} are independently hydrogen or unsubstituted alkyl.

Embodiment 43. The compound of embodiment 42, wherein R^{15A} and R^{15B} are independently unsubstituted methyl.

Embodiment44. The compound of embodiment 1, wherein the compound has the formula:

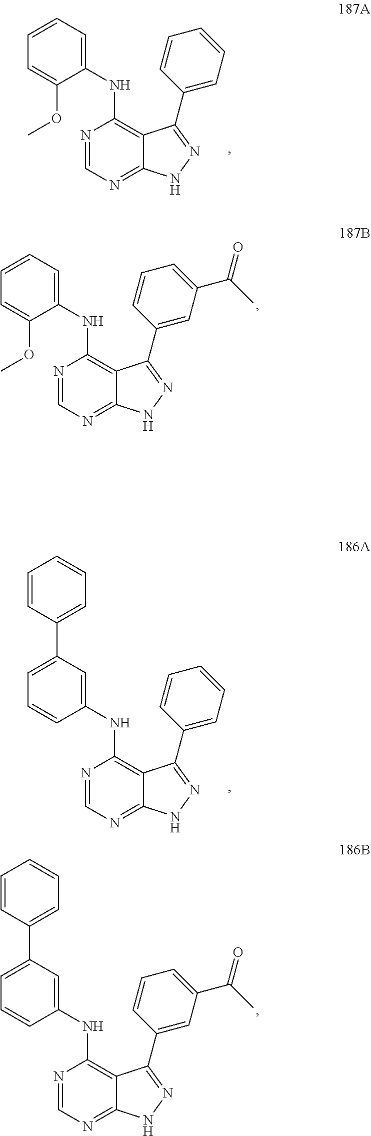 [SMILES: COc1ccccc1Nc1ncnc2[nH]nc(-c3ccccc3)c12 ; COc1ccccc1Nc1ncnc2[nH]nc(-c3cccc(C(C)=O)c3)c12 ; c1ccc(-c2cccc(Nc3ncnc4[nH]nc(-c5ccccc5)c34)c2)cc1 ; CC(=O)c1cccc(-c2n[nH]c3ncnc(Nc4cccc(-c5ccccc5)c4)c23)c1]

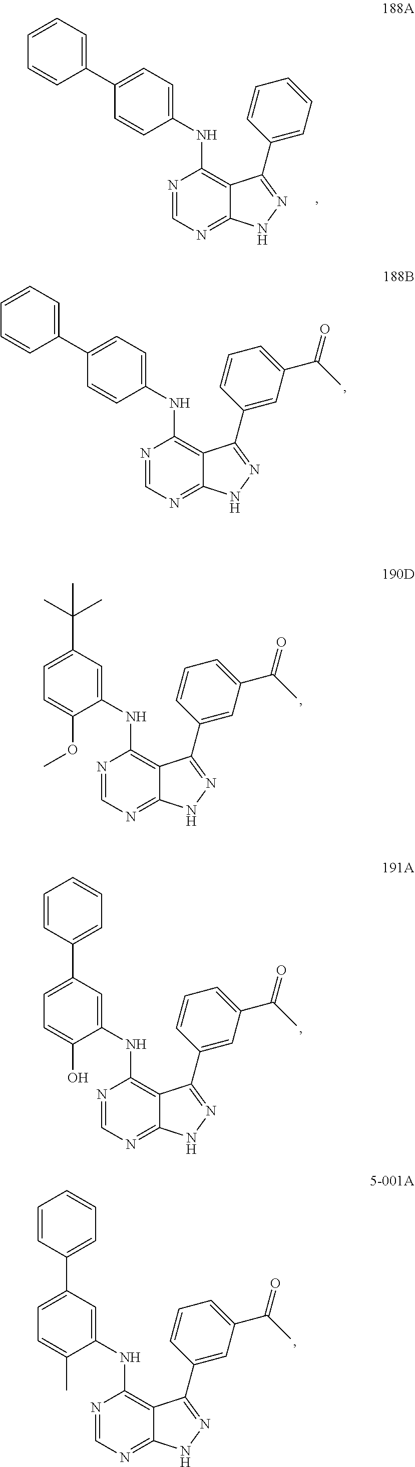 [SMILES: c1ccc(-c2ccc(Nc3ncnc4[nH]nc(-c5ccccc5)c34)cc2)cc1 ; CC(=O)c1cccc(-c2n[nH]c3ncnc(Nc4ccc(-c5ccccc5)cc4)c23)c1 ; COc1ccc(C(C)(C)C)cc1Nc1ncnc2[nH]nc(-c3cccc(C(C)=O)c3)c12 ; CC(=O)c1cccc(-c2n[nH]c3ncnc(Nc4cc(-c5ccccc5)ccc4O)c23)c1 ; CC(=O)c1cccc(-c2n[nH]c3ncnc(Nc4cc(-c5ccccc5)ccc4C)c23)c1]

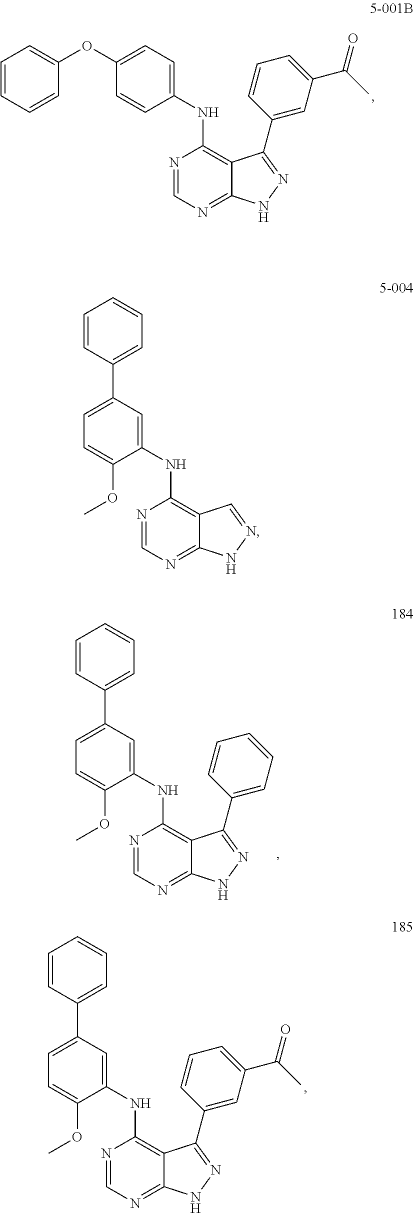 [SMILES: CC(=O)c1cccc(-c2n[nH]c3ncnc(Nc4ccc(Oc5ccccc5)cc4)c23)c1 ; COc1ccc(-c2ccccc2)cc1Nc1ncnc2[nH]ncc12 ; COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12]

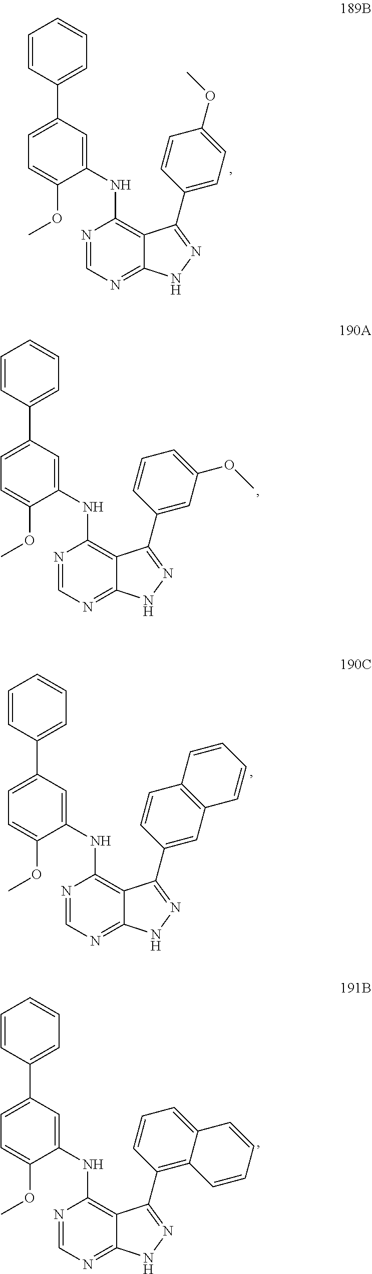 [SMILES: COc1ccc(-c2n[nH]c3ncnc(Nc4cc(-c5ccccc5)ccc4OC)c23)cc1 ; COc1cccc(-c2n[nH]c3ncnc(Nc4cc(-c5ccccc5)ccc4OC)c23)c1 ; COc1ccc(-c2ccccc2)cc1Nc1ncnc2[nH]nc(-c3ccc4ccccc4c3)c12 ; COc1ccc(-c2ccccc2)cc1Nc1ncnc2[nH]nc(-c3cccc4ccccc34)c12]

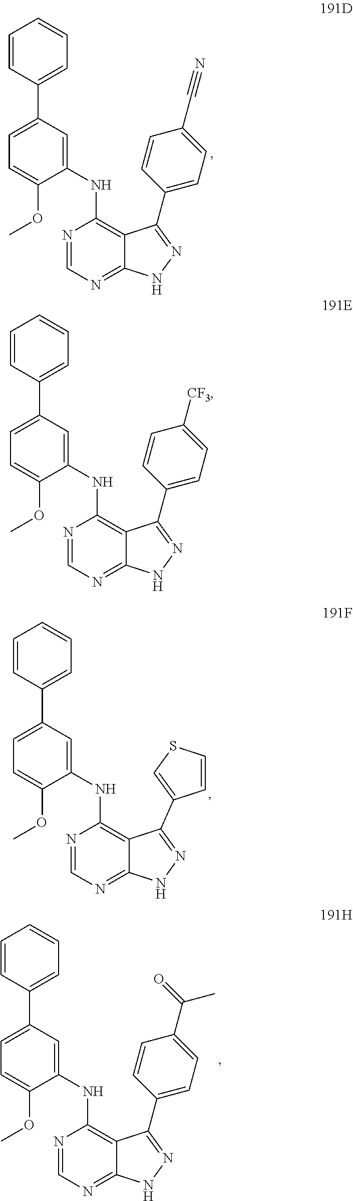 [SMILES: COc1ccc(-c2ccccc2)cc1Nc1ncnc2[nH]nc(-c3ccc(C#N)cc3)c12 ; COc1ccc(-c2ccccc2)cc1Nc1ncnc2[nH]nc(-c3ccc(C(F)(F)F)cc3)c12 ; COc1ccc(-c2ccccc2)cc1Nc1ncnc2[nH]nc(-c3ccsc3)c12 ; COc1ccc(-c2ccccc2)cc1Nc1ncnc2[nH]nc(-c3ccc(C(C)=O)cc3)c12]

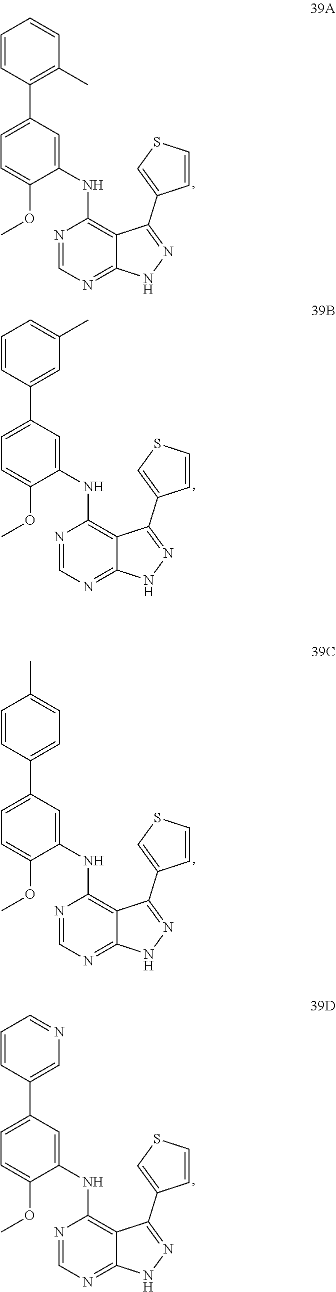

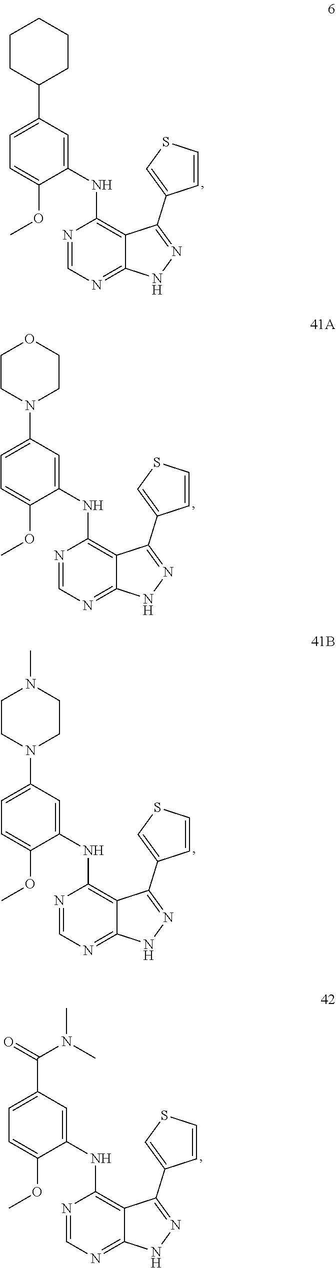 [SMILES: COc1ccc(C2CCCCC2)cc1Nc1ncnc2[nH]nc(-c3ccsc3)c12 ; COc1ccc(N2CCOCC2)cc1Nc1ncnc2[nH]nc(-c3ccsc3)c12 ; COc1ccc(N2CCN(C)CC2)cc1Nc1ncnc2[nH]nc(-c3ccsc3)c12 ; COc1ccc(C(=O)N(C)C)cc1Nc1ncnc2[nH]nc(-c3ccsc3)c12]

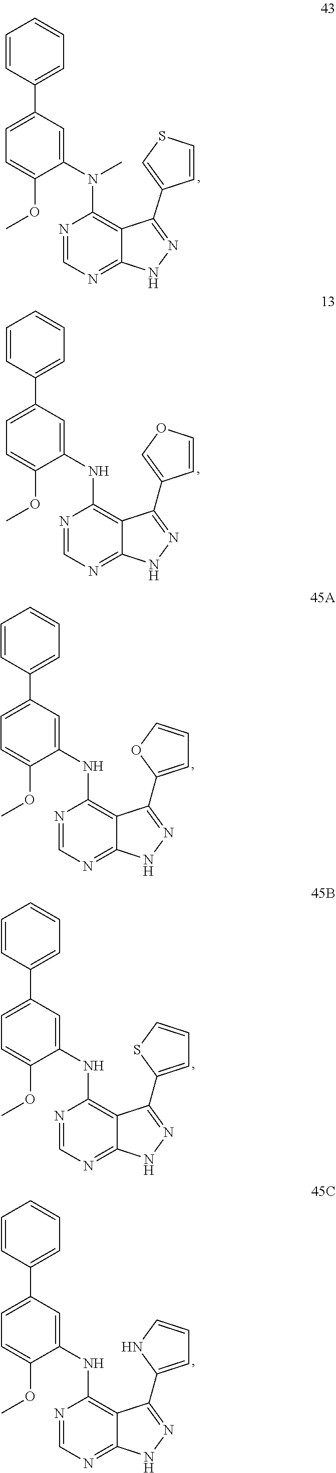 [SMILES: COc1ccc(-c2ccccc2)cc1N(C)c1ncnc2[nH]nc(-c3ccsc3)c12 ; COc1ccc(-c2ccccc2)cc1Nc1ncnc2[nH]nc(-c3ccoc3)c12 ; COc1ccc(-c2ccccc2)cc1Nc1ncnc2[nH]nc(-c3ccco3)c12 ; COc1ccc(-c2ccccc2)cc1Nc1ncnc2[nH]nc(-c3cccs3)c12 ; COc1ccc(-c2ccccc2)cc1Nc1ncnc2[nH]nc(-c3ccc[nH]3)c12]

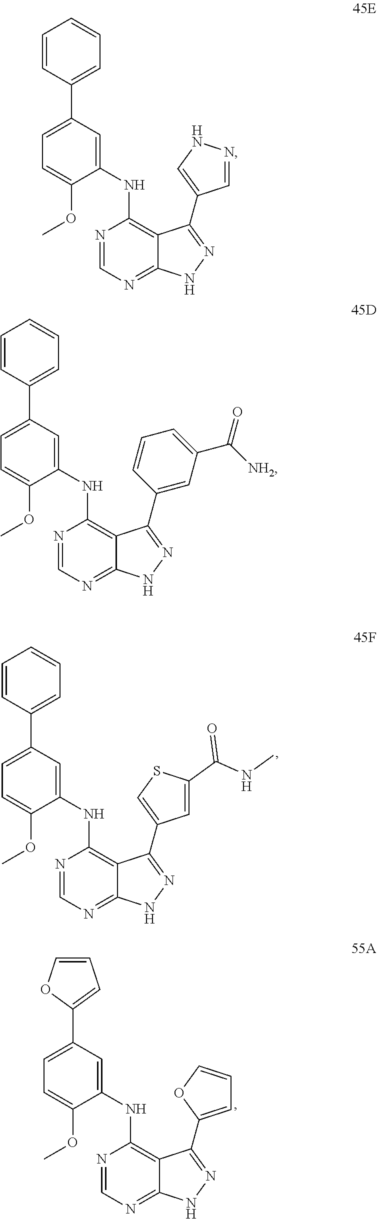

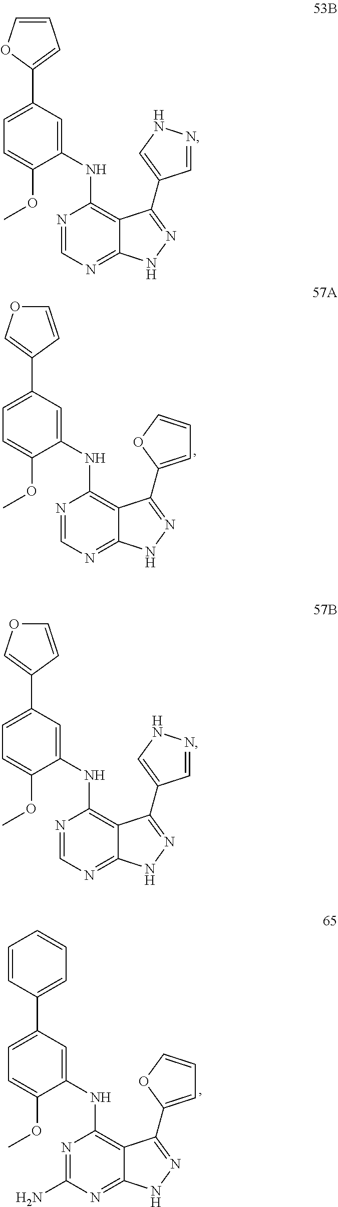 [SMILES: COc1ccc(-c2ccco2)cc1Nc1ncnc2[nH]nc(-c3cn[nH]c3)c12 ; COc1ccc(-c2ccoc2)cc1Nc1ncnc2[nH]nc(-c3ccco3)c12 ; COc1ccc(-c2ccoc2)cc1Nc1ncnc2[nH]nc(-c3cn[nH]c3)c12 ; COc1ccc(-c2ccccc2)cc1Nc1nc(N)nc2[nH]nc(-c3ccco3)c12]

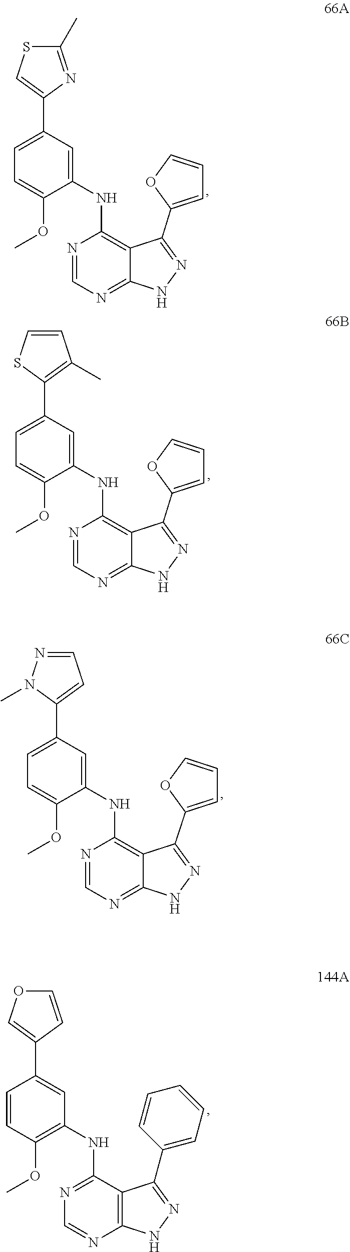 [SMILES: COc1ccc(-c2csc(C)n2)cc1Nc1ncnc2[nH]nc(-c3ccco3)c12 ; COc1ccc(-c2sccc2C)cc1Nc1ncnc2[nH]nc(-c3ccco3)c12 ; COc1ccc(-c2ccnn2C)cc1Nc1ncnc2[nH]nc(-c3ccco3)c12 ; COc1ccc(-c2ccoc2)cc1Nc1ncnc2[nH]nc(-c3ccccc3)c12]

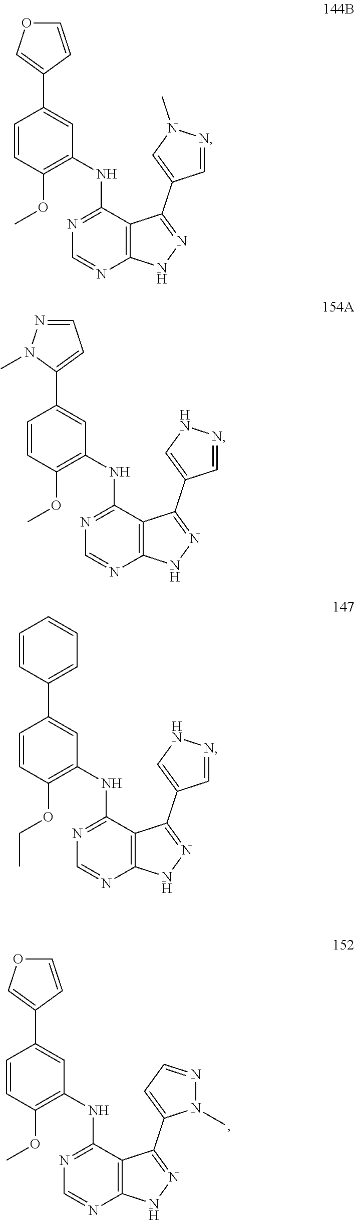 [SMILES: COc1ccc(-c2ccoc2)cc1Nc1ncnc2[nH]nc(-c3cnn(C)c3)c12 ; COc1ccc(-c2ccnn2C)cc1Nc1ncnc2[nH]nc(-c3cn[nH]c3)c12 ; CCOc1ccc(-c2ccccc2)cc1Nc1ncnc2[nH]nc(-c3cn[nH]c3)c12 ; COc1ccc(-c2ccoc2)cc1Nc1ncnc2[nH]nc(-c3ccnn3C)c12]

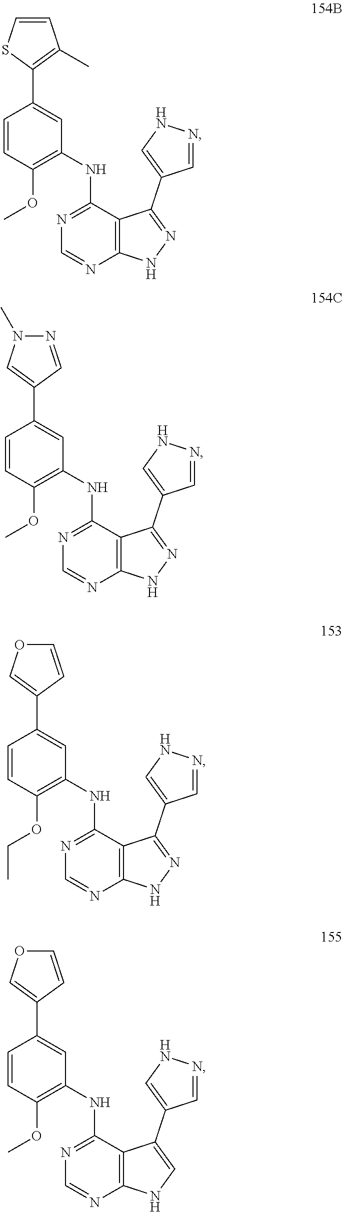 [SMILES: COc1ccc(-c2sccc2C)cc1Nc1ncnc2[nH]nc(-c3cn[nH]c3)c12 ; COc1ccc(-c2cnn(C)c2)cc1Nc1ncnc2[nH]nc(-c3cn[nH]c3)c12 ; CCOc1ccc(-c2ccoc2)cc1Nc1ncnc2[nH]nc(-c3cn[nH]c3)c12 ; COc1ccc(-c2ccoc2)cc1Nc1ncnc2[nH]cc(-c3cn[nH]c3)c12]

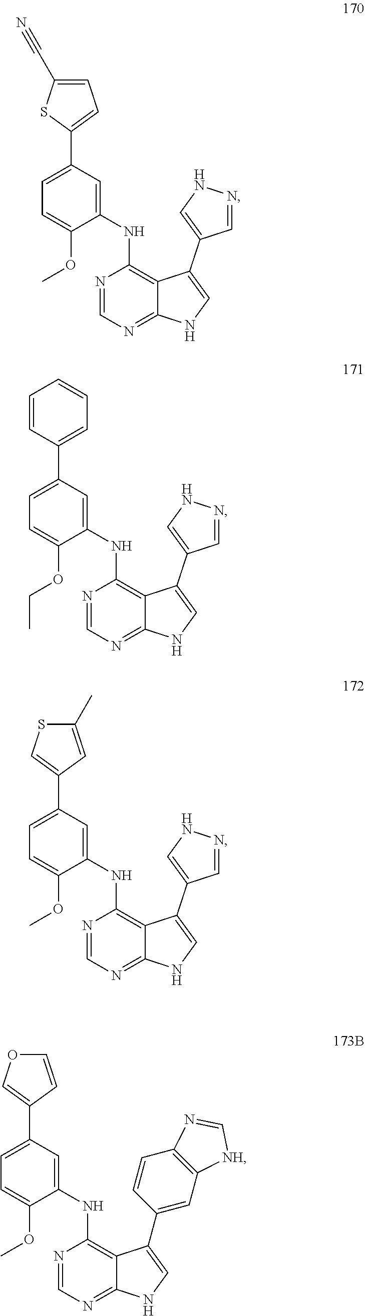 [SMILES: COc1ccc(-c2ccc(C#N)s2)cc1Nc1ncnc2[nH]cc(-c3cn[nH]c3)c12 ; CCOc1ccc(-c2ccccc2)cc1Nc1ncnc2[nH]cc(-c3cn[nH]c3)c12 ; COc1ccc(-c2csc(C)c2)cc1Nc1ncnc2[nH]cc(-c3cn[nH]c3)c12 ; COc1ccc(-c2ccoc2)cc1Nc1ncnc2[nH]cc(-c3ccc4nc[nH]c4c3)c12]

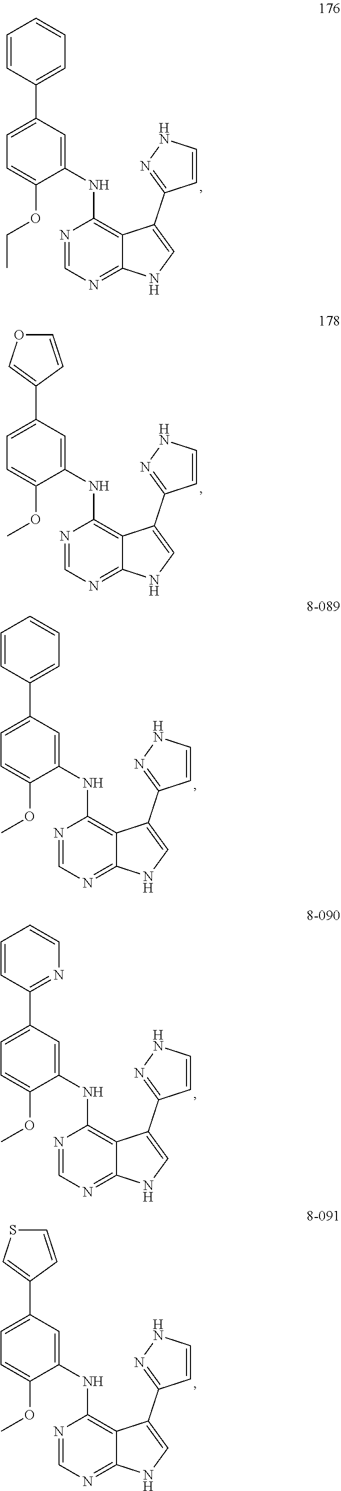 [SMILES: CCOc1ccc(-c2ccccc2)cc1Nc1ncnc2[nH]cc(-c3cc[nH]n3)c12 ; COc1ccc(-c2ccoc2)cc1Nc1ncnc2[nH]cc(-c3cc[nH]n3)c12 ; COc1ccc(-c2ccccc2)cc1Nc1ncnc2[nH]cc(-c3cc[nH]n3)c12 ; COc1ccc(-c2ccccn2)cc1Nc1ncnc2[nH]cc(-c3cc[nH]n3)c12 ; COc1ccc(-c2ccsc2)cc1Nc1ncnc2[nH]cc(-c3cc[nH]n3)c12]

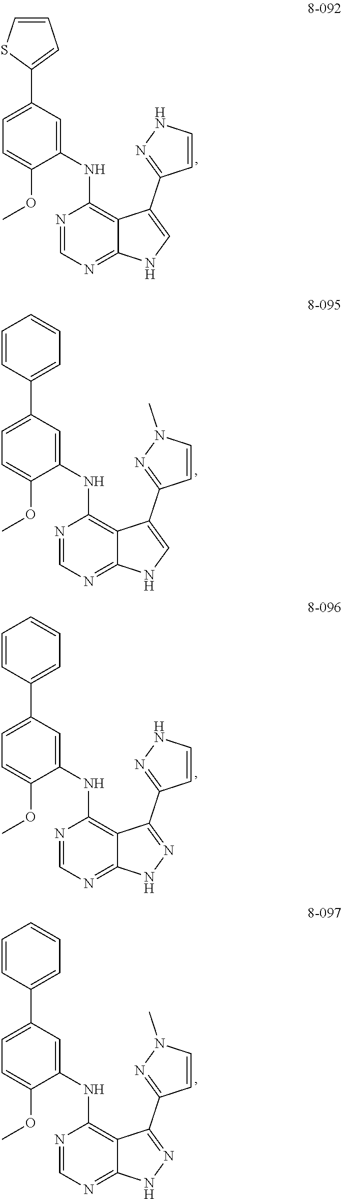 [SMILES: COc1ccc(-c2cccs2)cc1Nc1ncnc2[nH]cc(-c3cc[nH]n3)c12 ; COc1ccc(-c2ccccc2)cc1Nc1ncnc2[nH]cc(-c3ccn(C)n3)c12 ; COc1ccc(-c2ccccc2)cc1Nc1ncnc2[nH]nc(-c3cc[nH]n3)c12 ; COc1ccc(-c2ccccc2)cc1Nc1ncnc2[nH]nc(-c3ccn(C)n3)c12]

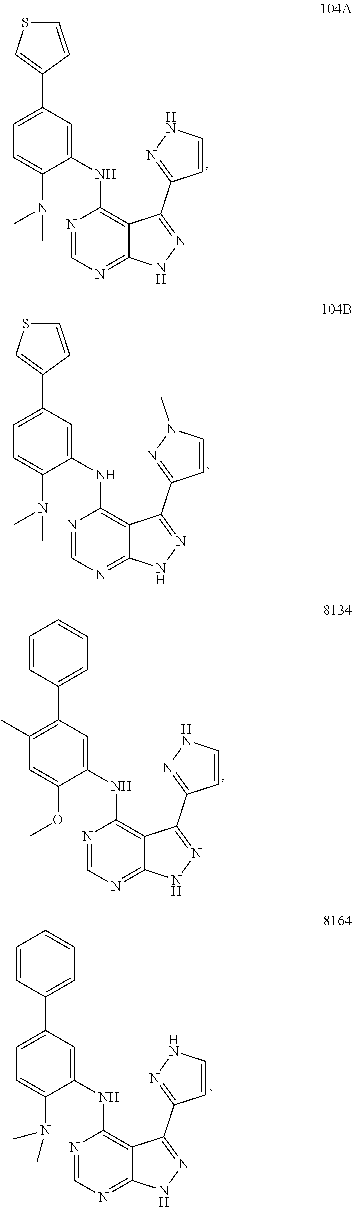 [SMILES: CN(C)c1ccc(-c2ccsc2)cc1Nc1ncnc2[nH]nc(-c3cc[nH]n3)c12 ; CN(C)c1ccc(-c2ccsc2)cc1Nc1ncnc2[nH]nc(-c3ccn(C)n3)c12 ; COc1cc(C)c(-c2ccccc2)cc1Nc1ncnc2[nH]nc(-c3cc[nH]n3)c12 ; CN(C)c1ccc(-c2ccccc2)cc1Nc1ncnc2[nH]nc(-c3cc[nH]n3)c12]

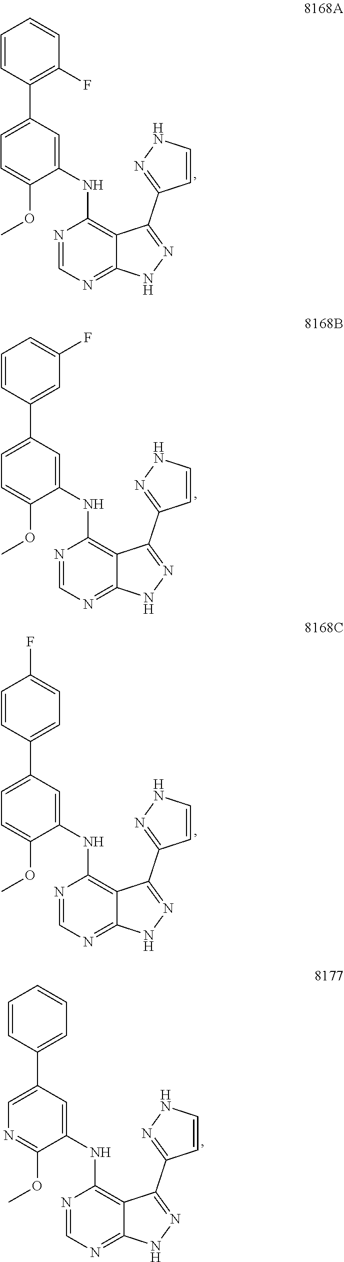 [SMILES: COc1ccc(-c2ccccc2F)cc1Nc1ncnc2[nH]nc(-c3cc[nH]n3)c12 ; COc1ccc(-c2cccc(F)c2)cc1Nc1ncnc2[nH]nc(-c3cc[nH]n3)c12 ; COc1ccc(-c2ccc(F)cc2)cc1Nc1ncnc2[nH]nc(-c3cc[nH]n3)c12 ; COc1ncc(-c2ccccc2)cc1Nc1ncnc2[nH]nc(-c3cc[nH]n3)c12]

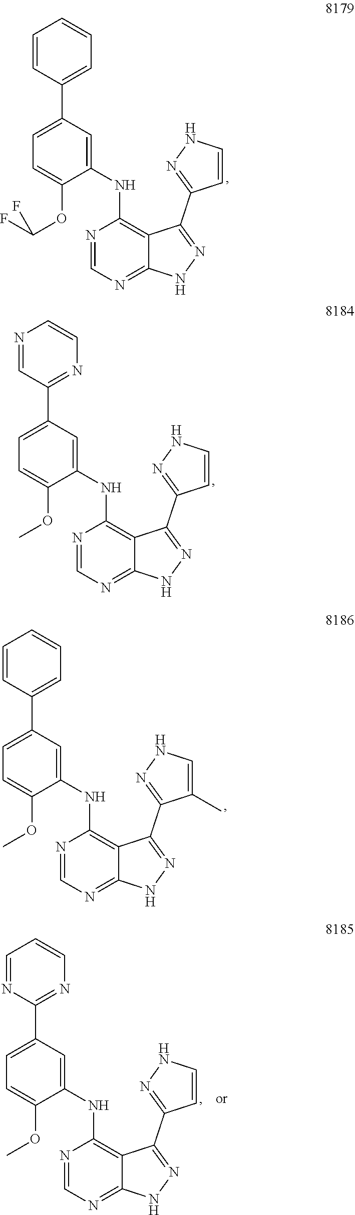 [SMILES: FC(F)Oc1ccc(-c2ccccc2)cc1Nc1ncnc2[nH]nc(-c3cc[nH]n3)c12 ; COc1ccc(-c2cnccn2)cc1Nc1ncnc2[nH]nc(-c3cc[nH]n3)c12 ; COc1ccc(-c2ccccc2)cc1Nc1ncnc2[nH]nc(-c3n[nH]cc3C)c12 ; COc1ccc(-c2ncccn2)cc1Nc1ncnc2[nH]nc(-c3cc[nH]n3)c12]

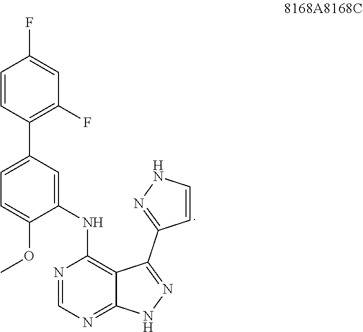 [SMILES: COc1ccc(-c2ccc(F)cc2F)cc1Nc1ncnc2[nH]nc(-c3cc[nH]n3)c12]

Embodiment 45. A pharmaceutical composition comprising a compound of one of embodiments 1 to 44 or a pharmaceutically acceptable salt thereof, and a pharmaceutically acceptable excipient.

Embodiment 46. The pharmaceutical composition of embodiment 45, further comprising an anti-cancer agent.

Embodiment 47. A method of treating a disease associated with HER2 activity in a patient in need of such treatment, said method comprising administering a therapeutically effective amount of a compound, or a pharmaceutically acceptable salt thereof, having the formula:

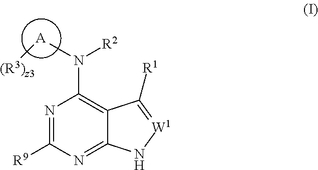

wherein Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment 48. A method of treating a disease associated with EGFR activity in a patient in need of such treatment, said method comprising administering a therapeutically effective amount of a compound, or a pharmaceutically acceptable salt thereof, having the formula:

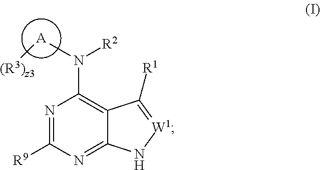

wherein; Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOᵥ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment 49. A method of treating cancer in a patient in need of such treatment, said method comprising administering a therapeutically effective amount of a compound, or a pharmaceutically acceptable salt thereof, having the formula:

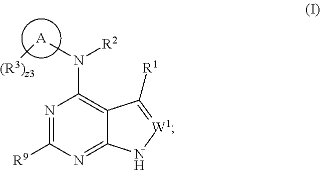

wherein; Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃R^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment 50. The method of embodiment 49, wherein the cancer is resistant to a HER2 inhibitor.

Embodiment 51. The method of embodiment 49, wherein the cancer is resistant to an EGFR inhibitor.

Embodiment 52. A method of inhibiting HER2 activity, said method comprising contacting HER2 with an effective amount of a compound, or a pharmaceutically acceptable salt thereof, having the formula:

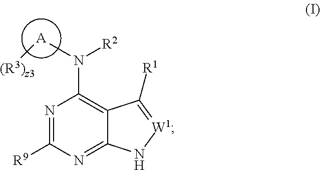

wherein; Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment 53. The method of embodiment 52, wherein HER2 is in an active conformation.

Embodiment 54. The method of embodiment 53, wherein HER2 is in a HER2-HER3 heterodimer.

Embodiment 55. A method of inhibiting EGFR activity, said method comprising contacting EGFR with an effective amount of a compound, or a pharmaceutically acceptable salt thereof, having the formula:

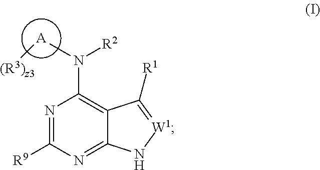

wherein; Ring A is aryl or heteroaryl; W¹ is N or C(H); R¹ is hydrogen, -L¹-L²-E, substituted or unsubstituted aryl, or substituted or unsubstituted heteroaryl; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}R^{3B}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment 56. The method of embodiment 55, wherein EGFR is in an active conformation.

Embodiment 57. A method of one of embodiments 47 to 56, wherein the compound has the formula

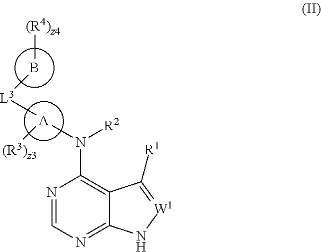

wherein Ring B is cycloalkyl, heterocycloalkyl, aryl, or heteroaryl; L³ is a bond, —S(O)₂—, —NR⁸—, —O—, —S—, —C(O)—, —C(O)NR⁸—, —NR⁸C(O)—, —NR⁸C(O)NH—, —NHC(O)NR⁸—, —C(O)O—, —OC(O)—, substituted or unsubstituted alkylene, or substituted or unsubstituted heteroalkylene; R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z4 is an integer from 0 to 5; Each R^{4A}, R^{4B}, R^{4C}, R^{4D}, R^{8A}, R^{8B}, R^{8C}, and R^{8D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl each X⁴ and X⁸ are independently —F, —Cl, —Br, or —I; n4 and n8 are independently an integer from 0 to 4; and m4, m8, v4, and v8, are independently an integer from 1 to 2.

Embodiment 58. A method of one of embodiments 47 to 56, wherein the compound has the formula:

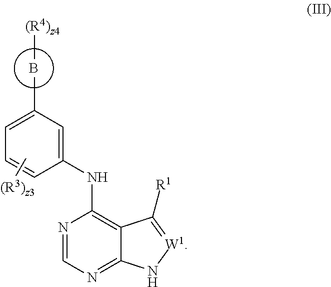

Embodiment 59. A method of one of embodiments 47 to 56, wherein the compound has the formula:

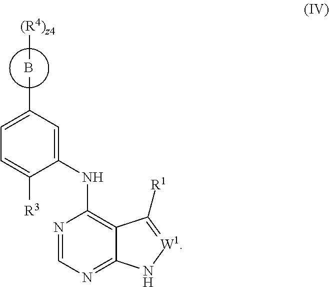

Embodiment 60. The method of one of embodiments 47 to 59, wherein R⁴ is independently halogen, —CX⁴₃, —CHX⁴₂, —CH₂X⁴, —OCX⁴₃, —OCH₂X⁴, —OCHX⁴₂, or —CN.

Embodiment 61. The method of one of embodiments 47 to 59, wherein R⁴ is independently halogen, —CX⁴₃, —CHX⁴₂, or —CH₂X⁴.

Embodiment 62. The method of one of embodiments 47 to 59, wherein R⁴ is independently halogen.

Embodiment 63. A method of one of embodiments 47 to 62, wherein the compound has the formula:

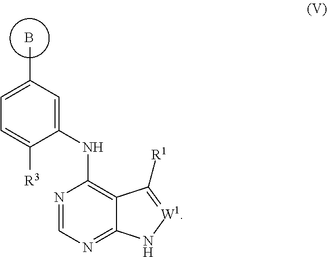

Embodiment 64. The method of one of embodiments 47 to 63, wherein W¹ is C(H).

Embodiment 65. The method of one of embodiments 47 to 63, wherein W¹ is N.

Embodiment 66. The method of one of embodiments 47 to 65, wherein R³ is an unsubstituted heteroalkyl.

Embodiment 67. The method of one of embodiments 47 to 65, wherein R³ is unsubstituted 2 to 5 membered heteroalkyl.

Embodiment 68. The method of one of embodiments 47 to 65, wherein R³ is —OCH₃, —OCH₂CH₃, —N(CH₃)₂, —NH₂, —NH(CH₃), —N(CH₂CH₃)₂, —NH(CH₂CH₃), —OCX³₃, —OCH₂X³, —OCHX³₂, or —SH.

Embodiment 69. The method of one of embodiments 48 to 68, wherein Ring B is substituted or unsubstituted aryl or heteroaryl.

Embodiment 70. The method of one of embodiments 48 to 68, wherein Ring B is substituted or unsubstituted phenyl or substituted or unsubstituted 5 to 6 membered heteroaryl.

Embodiment 71. The method of one of embodiments 48 to 68, wherein Ring B is substituted or unsubstituted phenyl.

Embodiment 72. The method of one of embodiments 48 to 68, wherein Ring B is substituted or unsubstituted 5 to 6 membered heteroaryl.

Embodiment 73. The method of one of embodiments 48 to 68, wherein Ring B is substituted or unsubstituted pyrazinyl, pyrimidinyl, furanyl, thienyl, pyrrolyl, imidazolyl, pyrazolyl, oxazolyl, isoxazolyl, or thiazolyl.

Embodiment 74. The method of one of embodiments 47 to 73, wherein R¹ is substituted or unsubstituted aryl or substituted or unsubstituted heteroaryl.

Embodiment 75. The method of one of embodiments 47 to 73, wherein R¹ is substituted or unsubstituted phenyl or substituted or unsubstituted 5 to 6 membered heteroaryl.

Embodiment 76. The method of one of embodiments 47 to 73, wherein R¹ is substituted or unsubstituted phenyl.

Embodiment 77. The method of one of embodiments 47 to 73, wherein R¹ is an unsubstituted phenyl.

Embodiment 78. The method of one of embodiments 47 to 73, wherein R¹ is a substituted or unsubstituted 5 to 6 membered heteroaryl.

Embodiment 79. The method of one of embodiments 47 to 73, wherein R¹ is an unsubstituted 5 to 6 membered heteroaryl.

Embodiment 80. The method of one of embodiments 47 to 73, wherein R¹ is substituted or unsubstituted pyridinyl, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted oxazolyl, substituted or unsubstituted isoxazolyl, or substituted or unsubstituted thiazolyl.

Embodiment 81. The method of one of embodiments 47 to 73, wherein R¹ is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

Embodiment 82. The method of one of embodiments 47 to 81, wherein R¹ is -L¹-L²-E.

Embodiment 83. The method of one of embodiments 47 to 82, wherein L¹ is a bond, —C(O)—, substituted or unsubstituted alkylene, or substituted or unsubstituted heteroalkylene.

Embodiment 84. The method of one of embodiments 47 to 82, wherein L¹ is a substituted or unsubstituted C₁-C₄ alkylene.

Embodiment 85. The method of one of embodiments 47 to 82, wherein L¹ is —C(O)CH₂CH₂CH₂—, —C(O)CH₂CH₂—, or —C(O)CH₂—.

Embodiment 86. The method of one of embodiments 47 to 85, wherein L² is —NR⁷—, substituted or unsubstituted heteroalkylene, or substituted or unsubstituted heterocycloalkylene.

Embodiment 87. The method of one of embodiments 47 to 85, wherein L² is —NH—.

Embodiment 88. The method of one of embodiments 47 to 87, wherein E is a covalent cysteine modifier moiety.

Embodiment 89. The method of one of embodiments 47 to 87, wherein E is:

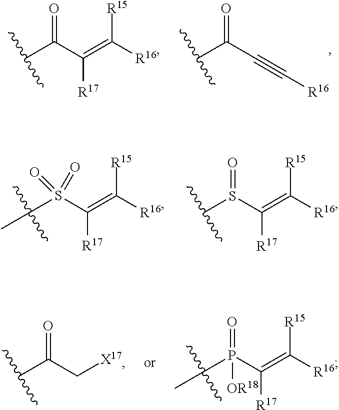

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; each X, X¹⁵, X¹⁶, X¹⁷ and X¹⁸ is independently —F, —Cl, —Br, or —I; n15, n16, n17, v15, v16, and v17, are independently an integer from 0 to 4; and m15, m16, and m17 are independently and integer from 1 to 2.

Embodiment 90. The method of embodiment 89, wherein R¹⁵, R¹⁶, R¹⁷, and R¹⁸ are hydrogen.

Embodiment 91. The method of one of embodiments 89 to 90, wherein E is:

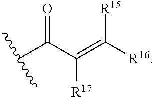

Embodiment 92. The method of embodiment 91, wherein R¹⁵ is hydrogen; R¹⁶ is hydrogen, —CH₃, or —CH₂NR^{16A}R^{16B}, R¹⁷ is hydrogen; and R^{16A} and R^{16B} are independently hydrogen or unsubstituted alkyl.

Embodiment 93. The method of embodiment 92, wherein R^{16A} and R^{16B} are independently unsubstituted methyl.

Embodiment 94. The method of embodiment 91, wherein R¹⁵ is hydrogen; R¹⁶ is hydrogen; R¹⁷ is hydrogen, —CH₃, or —CH₂NR^{17A}R^{17B}, and R^{17A} and R^{17B} are independently hydrogen or unsubstituted alkyl.

Embodiment 95. The method of embodiment 94, wherein R^{17A} and R^{17B} are independently unsubstituted methyl.

Embodiment 96. The compound of embodiment 91, wherein R¹⁵ is hydrogen, —CH₃, or —CH₂NR^{15A}R^{15B}, R¹⁶ is hydrogen; R¹⁷ is hydrogen; and R^{15A} and R^{15B} are independently hydrogen or unsubstituted alkyl.

Embodiment 97. The method of embodiment 96, wherein R^{15A} and R^{15B} are independently unsubstituted methyl.

Embodiment 98. An EGFR protein covalently bonded to a compound having the formula:

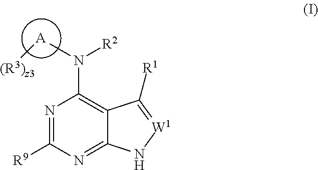

wherein Ring A is aryl or heteroaryl; W¹ is N or C(H) R¹ is -L¹-L²-E; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

Embodiment 99. A HER2 protein covalently bonded to a compound having the formula:

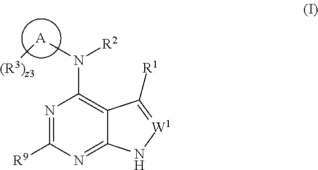

wherein Ring A is aryl or heteroaryl; W¹ is N or C(H) R¹ is -L¹-L²-E; R² is hydrogen, —CX²₃, —CHX²₂, —CH₂X², substituted or unsubstituted alkyl, or substituted or unsubstituted heteroalkyl; R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 4; L¹ is a bond, —S(O)₂—, —S(O)₂-Ph-, —NR⁶—, —O—, —S—, —C(O)—, —C(O) NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; E is an electrophilic moiety; each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl; each X, X³, X⁶, X⁷, and X⁹ is independently —F, —Cl, —Br, or —I; n3, n6, n7, and n9 are independently an integer from 0 to 4; and m3, m6, m7, m9, v3, v6, v7, and v9, are independently an integer from 1 to 2.

It is understood that the examples and embodiments described herein are for illustrative purposes only and that various modifications or changes in light thereof will be suggested to persons skilled in the art and are to be included within the spirit and purview of this application and scope of the appended embodiments. All publications, patents, and patent applications cited herein are hereby incorporated by reference in their entirety for all purposes.

### EXAMPLES

A. Overcoming Resistance to HER2 Inhibitors

Within the EGFR family, HER2 and HER3 are unique. HER3 is classified as a pseudokinase with only residual kinase activity, whereas HER2 has no known activating ligand but is constitutively able to dimerize with other active family members. In this way HER2 and HER3 together form a functional RTK unit, with HER3 responding to activating ligands such as neuregulin (NRG), HER2 providing the intracellular kinase activity, and both intracellular domains providing phosphorylation sites. Additionally, HER2 and HER3 are each other's preferred heterodimerization partners and also form the most mitogenic complex among all possible EGFR family dimers[11]. Because of this co-dependence, HER3 is equally important for the formation, proliferation, and survival of HER2 overexpressing tumors[12].

Although HER2 amplification and overexpression is the most well studied means of oncogenic activation of the HER2/HER3 heterodimer, improper signaling can also be caused by secretion of the HER3 ligand NRG to stimulate HER2/HER3 heterodimers in an autocrine manner as well as by mutations in HER3 that stabilize and activate heterodimers independently of ligand[13, 14]. In addition, mutations that activate the HER2 kinase domain have also been reported[15-17].

Inhibition of HER2 using small molecule kinase inhibitors such as lapatinib or HER2-targeted antibodies such as ado-trastuzumab emtansine (T-DM1), have shown efficacy against HER2-driven cancers in the clinic[18, 19]. However, recent studies have demonstrated that the presence of NRG induces resistance against currently-approved HER2 targeted mono-therapies through HER2/HER3 signaling[20, 21]. Additionally, inhibition of HER2/HER3 signaling at either the RTK level or of the downstream PI3K/Akt pathway releases a negative feedback loop that increases the transcription, translation, and membrane localization of HER3 [22-24]. This increase in the level of HER3 causes a rebound in its phosphorylation and reactivation of the PI3K/Akt pathway even in the continued presence of lapatinib, indicating that formation of HER2/HER3 heterodimers is crucial for intrinsic cellular resistance to current HER2 targeted therapies[25]. This severe limitation illustrates why more effective therapies targeting the active HER2/HER3 dimer are required.

Here, we evaluated the ability of current reversible HER2 inhibitors to inhibit signaling and proliferation in cancer cell lines driven by HER2/HER3 heterodimers activated by different oncogenic mechanisms. Across several cell lines, stabilization of HER2 in the active conformation led to severely diminished activity of both lapatinib and TAK-285. We therefore aimed to identify a novel HER2/HER3 inhibitor that would preferentially target the active state of the heterodimer. Reasoning that a biochemical screen would be unable to capture the relevant cellular conformation of the fully formed transmembrane complex, we turned to a cell based screening strategy. A high throughput screen of 950,000 small molecules against an engineered Ba/F3 cell line dependent on NRG stimulated HER2/HER3 heterodimers yielded a hit scaffold that we optimized to create a next generation HER2 inhibitor. The optimized inhibitor is capable of potently inhibiting signaling from the HER2/HER3 heterodimer regardless of the activating oncogenic mechanism.

B. Active HER2/HER3 Heterodimers are Resistant to DFG In/α-C Out Binding Inhibitors

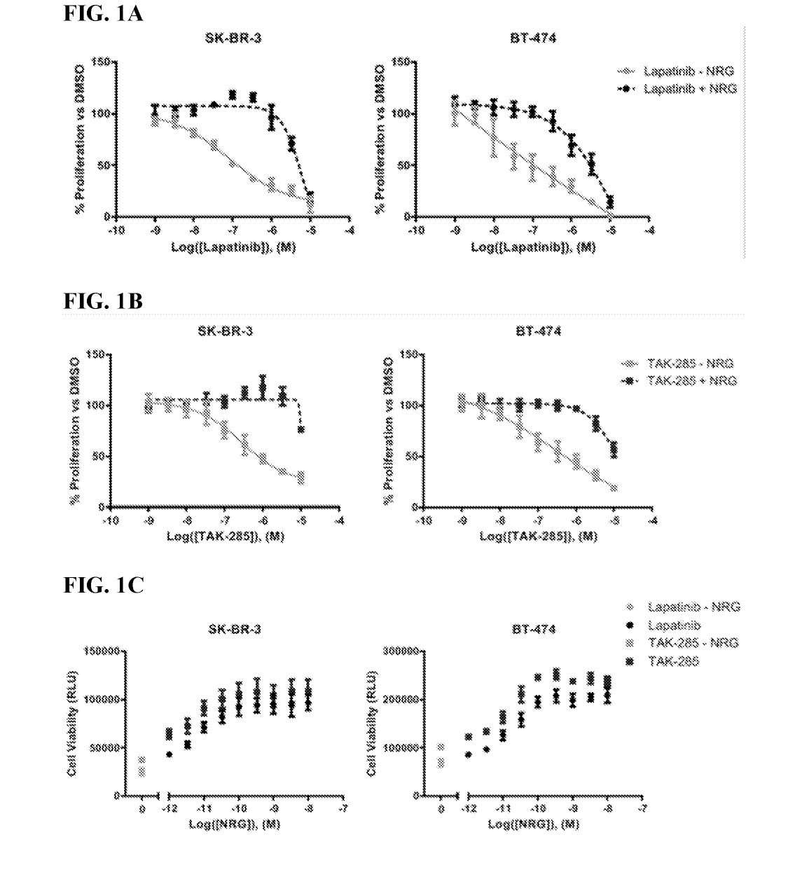

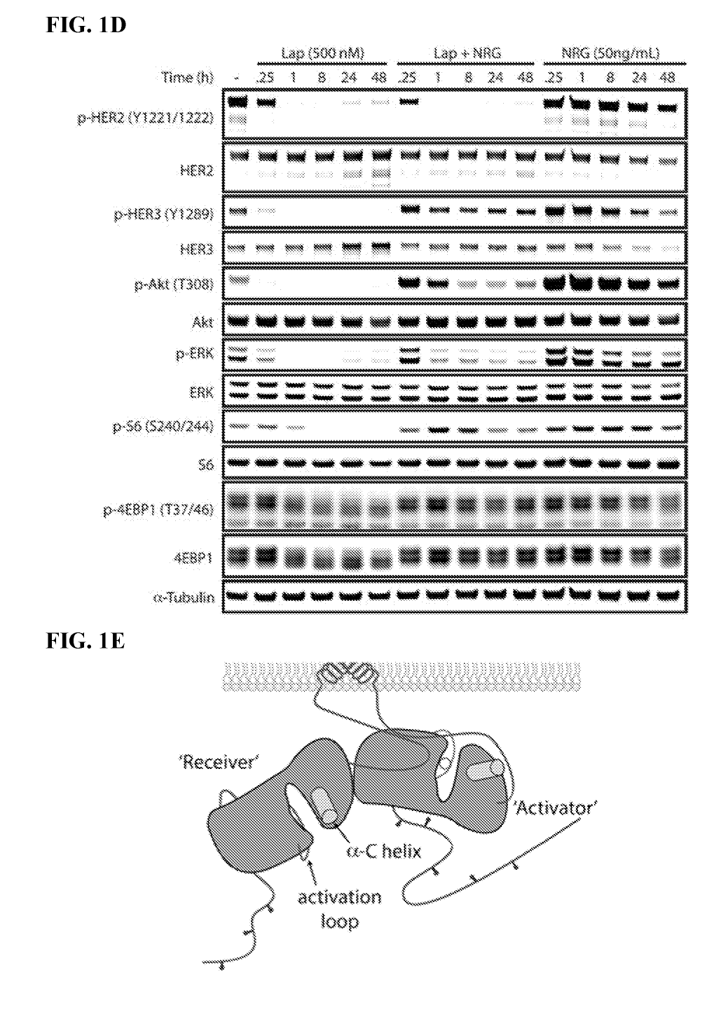

We first confirmed that the addition of the HER3 activating ligand NRG 1, hereafter referred to as NRG, is able to dramatically rescue the proliferation of HER2 over-expressing breast cancer cell lines treated with the HER2 kinase inhibitors lapatinib and TAK-285 (FIGS. 1A-1B). This rescue of cell proliferation was dose dependent and effective at pM concentrations of NRG in the presence of either HER2 inhibitor at 1 (FIG. 1C). To determine how NRG was able to so profoundly rescue cellular proliferation we examined a time course of HER2/HER3 signaling in SK-BR-3 cells exposed to either lapatinib, NRG, or the combination of the two. While lapatinib alone was able to rapidly and sustainably inhibit all signaling from HER2 and HER3, the addition of NRG prevented the complete inhibition of p-HER3 and all downstream signaling pathways at all time points examined, although HER2 phosphorylation remained inhibited (FIG. 1D). Analysis of signaling from alternative EGFR family members that could potentially contribute to this phenotype revealed that p-EGFR actually decreased in response to NRG and was still able to be inhibited by lapatinib while HER4, which is thought to function as a tumor suppressor, was undetectable in this cell line [26, 27] (FIG. 8).

The ability of NRG to rescue phosphorylation of HER3, but not of HER2, in the presence of lapatinib could have several origins. The very weak kinase activity of HER3 itself is unlikely to be sufficient—although it is not inhibited by lapatinib. A more likely explanation is that NRG-induced heterodimerization of HER2 and HER3 stabilizes a conformation of the HER2 kinase domain that is resistant to inhibition by the inhibitors tested. Both lapatinib and TAK-285 bind to the DFG in/α-C out kinase conformation and occupy the back hydrophobic pocket of HER2 that is only available when the kinase domain is in an inactive conformation with the αC-helix in the characteristic ‘out’ position. This mode of binding has the advantage of giving these inhibitors slow off-rates and increased selectivity, but could also explain their ineffectiveness in the presence of NRG. Crystal structures of EGFR family homodimers and of a HER3/EGFR kinase complex have shown that only the ‘activator’ kinase in the asymmetric dimer can retain the inactive conformation (FIG. 1E) [30, 31]. In HER2/HER3 complexes HER3 will adopt this position exclusively, whereas HER2 will take the ‘receiver’ kinase position and become stabilized in the active conformation. Thus, in a HER2/HER3 complex, the size and accessibility of the back hydrophobic pocket of the HER2 kinase domain will be greatly reduced, preventing lapatinib or TAK-285 from binding.

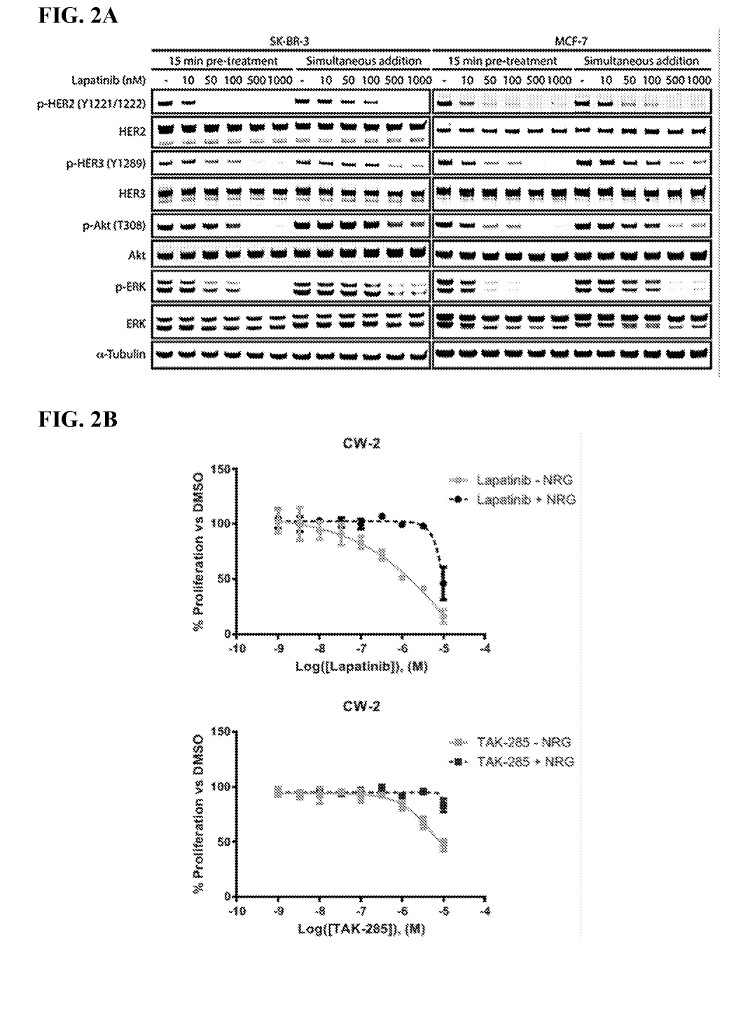

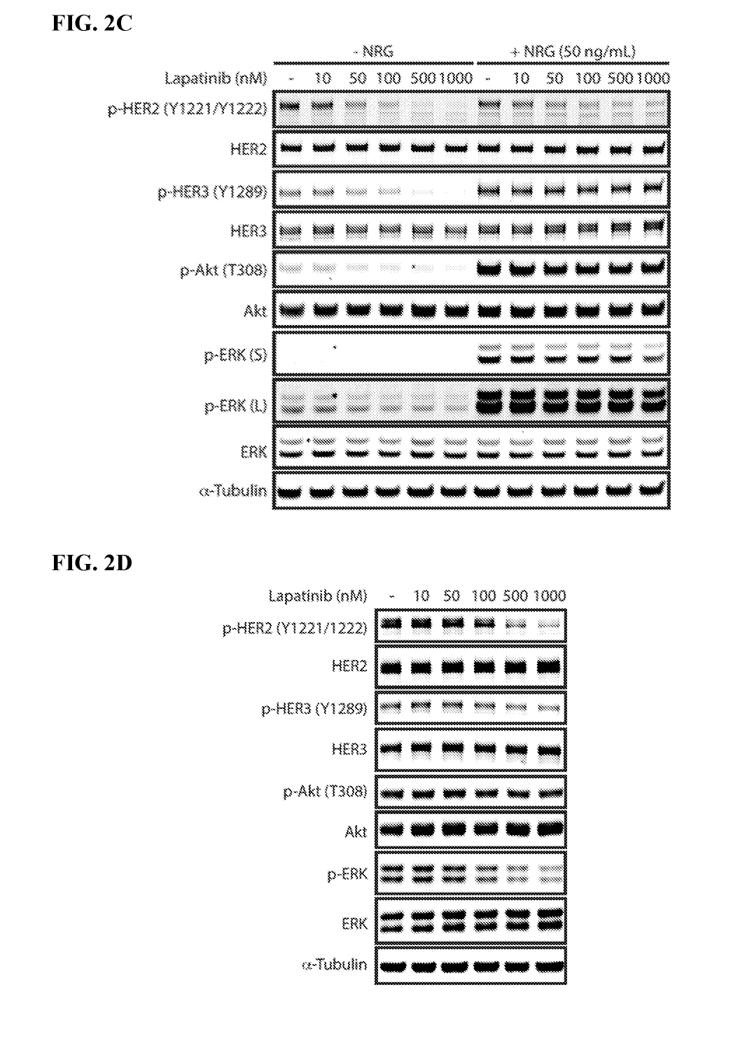

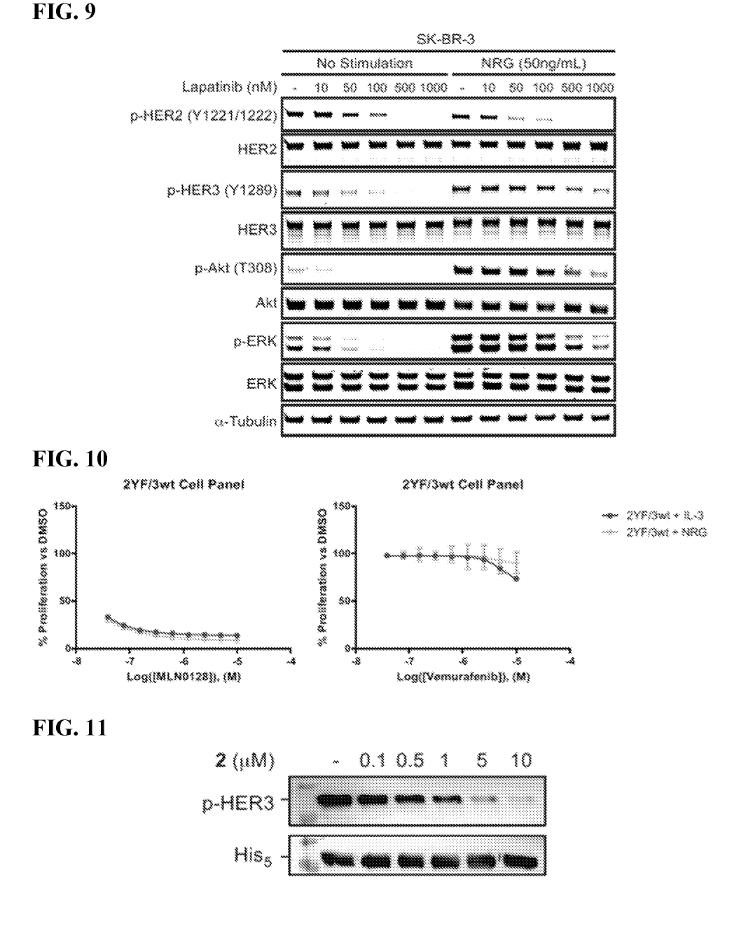

To determine if NRG induced heterodimerization prevents lapatinib binding, we treated serum-starved SK-BR-3 cells with lapatinib either 15 min before or simultaneously with NRG stimulation, and then rapidly examined HER2/HER3 signaling to monitor the on-rate of lapatinib in these cells. Lapatinib concentrations greater than 100 nM were sufficient to inhibit the entire RTK signaling pathway when added before NRG (FIG. 2A), i.e. in the absence of HER2/HER3 heterodimers. By contrast, lapatinib was much less effective when added simultaneously with NRG—where lapatinib would need to bind to the NRG-induced HER2/HER3 heterodimer to inhibit signaling—with significant levels of p-HER3, p-Akt and p-Erk remaining even in the presence of 1 μM lapatinib (FIG. 2A). A similar trend was seen in MCF-7 cells, which express modest levels of HER2 and HER3, demonstrating that this effect does not require HER2 overexpression (FIG. 2A). The same trend was also observed when cells were first treated with either NRG or vehicle for 15 min followed by 15 min of lapatinib treatment (FIG. 9).

To assess if NRG's ability to rescue the cellular proliferation of cell lines where the HER2/HER3 dimer may be activated by alternative mechanisms we assessed signaling and proliferation in CW-2 cells, which contain an activating mutation in the C-lobe of HER3 (E928G) as well as an N-lobe mutation in HER2 (L755S). Such HER3 mutations have been reported to activate HER2 by stabilizing HER2/HER3 heterodimers independently of NRG[13, 30]. Similar to the HER2 overexpressing cell lines, the addition of NRG rescued the viability of the HER3 mutant line in a 72 h proliferation assay from both HER2 inhibitors and rescued HER2/HER3 signaling from lapatinib even after 1 h of drug exposure (FIGS. 2B-2C).

We next sought to determine whether this apparent resistance to lapatinib is specific to NRG-induced HER2/HER3 heterodimers or if mutations that bias HER2 towards its active conformation in the absence of NRG can elicit a similar effect. We monitored HER2 and HER3 signaling 15 min after the addition of lapatinib in the lung cancer cell line NCI-H1781, which contains an insertion in the HER2 kinase domain at a position known to increase HER2 kinase activity[16]. As shown in FIG. 2D, lapatinib, even at a concentration of was unable to fully inhibit signaling demonstrating that this activating mutation is sufficient to hinder lapatinib binding to HER2.

Taken together, these data argue that NRG rescues HER2/HER3 driven cells from DFG in/α-C out inhibitors by stabilizing HER2 in the active conformation within HER2/HER3 heterodimers. This inability to directly target the active state of HER2 places a major limitation on the effectiveness of a majority of current HER2 inhibitors since inhibition of HER2/HER3 signaling triggers feedback loops that lead to the increased membrane expression of HER2 and HER3, which increases the number of active HER2/HER3 heterodimers that then rescue signaling and proliferation [22, 23, 25]. Our findings indicate that inhibitors which target the active HER2/HER3 heterodimer will have significant advantages, especially in situations that increase the number of active HER2/HER3 heterodimers.

C. Identification of a Novel Inhibitor of HER2/HER3 Heterodimers

In order to find a small molecule inhibitor capable of binding to the active HER2/HER3 signaling complex we developed a high throughput cellular screen using a Ba/F3 cell line engineered to be dependent on NRG-induced HER2/HER3 heterodimers. Ba/F3 cells are normally dependent on IL-3 signaling for their proliferation and survival, but can be made dependent on introduced oncogenic signals[32]. We sequentially selected transduced Ba/F3 cells for populations stably expressing HER3 and then HER2. To ensure that all of the proliferative signal could be attributed to HER2/HER3 heterodimers, rather than HER2 homodimers, the 9 C-terminal tyrosines in HER2 were mutated to phenylalanine (HER2YF). We then withdrew IL-3 and supplemented the media with NRG to select for NRG dependent HER2YF/HER3wt cells (2YF/3wt). The resulting 2YF/3wt cell line was completely dependent on NRG for survival and allowed us to screen for inhibitors of full length HER2/HER3 heterodimers in their native cellular conformations using a cell viability assay. In addition to allowing us to identify potential inhibitors of active HER2, this cellular system also has the potential to uncover compounds with novel mechanisms of action—such as inhibitors of HER3's ability to allosterically activate HER2. This system could also identify inhibitors of HER3's weak kinase activity in the unlikely event that this is important for proliferation of 2YF/3wt cells.

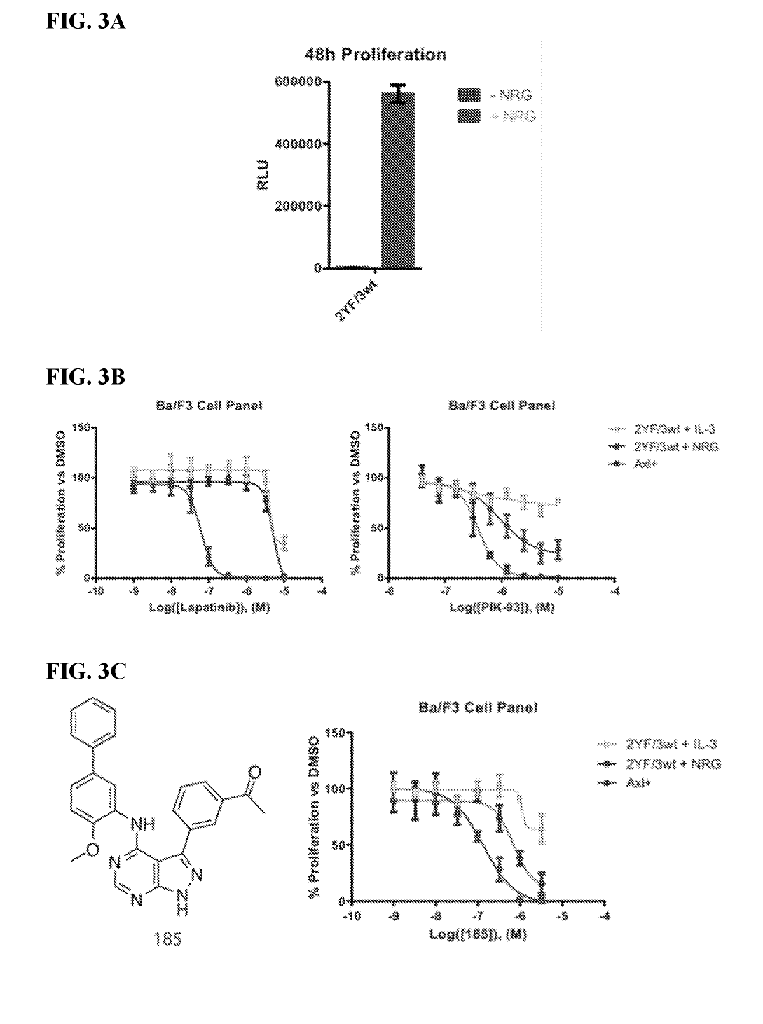

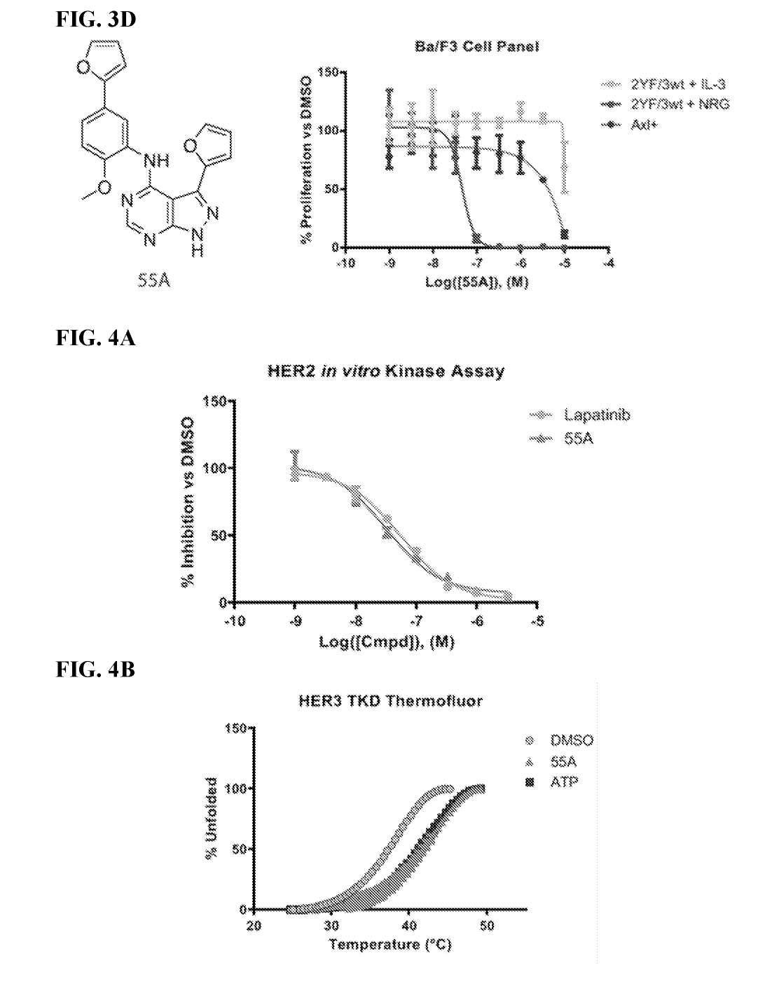

To validate the screen and test the effectiveness of counter-screening with either the same 2YF/3wt Ba/F3 cell line or the parental Ba/F3's in the presence of IL-3 to remove cytotoxic primary hits, we first tested a panel of kinase inhibitors with established mechanisms of action. This panel of inhibitors demonstrated that MAPK pathway inhibitors (e.g. vemurafenib) would not score as hits in NRG-treated 2YF/3wt cells, whereas mTOR inhibitors (e.g. MLN0128) are ruled out in our toxicity counter screen, showing equal activity in the presence of either NRG or IL-3 (FIG. 10). By contrast, HER2 and PI3K inhibitors showed a clear window for selective inhibition of NRG driven cells over IL-3 driven cells (FIG. 3B). In order to rapidly remove any hit compounds that do not directly inhibit the HER2/HER3 heterodimer, we created a separate Ba/F3 cell line dependent on the overexpression of full-length wt Axl, which also signals through the PI3K pathway. As shown in FIG. 3B, this panel of Ba/F3 cell lines was suitable to segregate lapatinib from the PI3K inhibitor PIK-93.

The 48 h proliferation assay of 2YF/3wt+NRG cells was miniaturized and optimized for 1536 well plates, which we used to screen a diverse collection of 950,000 drug-like molecules (Z′≥0.75). This primary screen resulted in 14,012 hits (>50% inhibition vs DMSO) that was reduced to 1,423 compounds after triplicate confirmation and the parental Ba/F3+IL-3 counter-screen (<30% inhibition vs DMSO), which were then assayed in dose response against all 3 cell lines (2YF/3wt+NRG, parental Ba/F3+IL-3, and Axl+) (Table 1).

TABLE 1
Small molecule screening data

| Category | Parameter | Description |
|---|---|---|
| Assay | Type of assay | Cell-based |
| | Target | ERBB3 (UniProt P21860, KEGG |
| | | 2065), ERBB2 (UniProt P04626, |
| | | KEGG 2064) |
| | Primary measurement | Detection of cell viability |
| | Key reagents | CellTiter-Glo Luminescent Cell |
| | | Viability Assay (Promega) |
| | Assay protocol | Described in the Methods section |
| | Additional comments | |
| Library | Library size | 950,000 compounds |
| | Library composition | Diverse set of small-molecule |
| | | compounds, composing of known |
| | | drug library, bioactive library |
| | | with known mechanism of action |
| | | (MOA) and also 750,000 proprietary |
| | | compounds with unknown MOA |
| | Source | Commercially available and |
| | | proprietary sources |
| | Additional comments | The compounds were stored at room |
| | | temperature for max 6 months during |
| | | HTS |
| Screen | Format | White 1,536-well solid bottom plates |
| | | from Greiner or Corning |
| | Concentration(s) tested | 4 μM compound, 0.4% DMSO |
| | Plate controls | 4 uM lapatinib, 0.4% DMSO |
| | Reagent/compound | Compounds: Echo 555 Liquid |
| | dispensing system | Handler from Labcyte; cells, |
| | | Cell Titer-Glo reagent: Bottle |
| | | valve dispenser from Kalypsys |
| | Detection instrument | ViewLux uHTS Microplate Imager |
| | and software | (PerkinElmer) |
| | Assay validation/QC | Z′ ≥ 0.75 |
| | Correction factors | — |
| | Normalization | DMSO control |
| | Additional comments | |
| Post-HTS | Hit criteria | >50% inhibition of the luminescence |
| analysis | | signal as compared to the DMSO |
| | | control |
| | Hit rate | 1.5% |
| | Additional assay(s) | Hit confirmation in triplicate with the |
| | | screening assay. Counter screens: |
| | | Ba/F3 and Ba/F3 Axl cell viability |
| | Confirmation of hit | Compounds were re-synthesized |
| | purity and structure | |

Compounds were first screened for the ability to inhibit the proliferation of the 2YF/3wt+NRG cells, counter-screened against the parental cells in the presence of IL-3, and then screened in dose response against all 3 cell lines. This screening and triaging process led to the identification of 3 compounds that share the same scaffold, exemplified by compound 1 (also referred to herein as compound 185) in FIG. 3C, which reproducibly showed preferential inhibition of 2YF/3wt+NRG cell proliferation. Optimization of the scaffold through 5 iterations of analog synthesis, each consisting of approximately 10 compounds, led to exemplary compound 2 (also referred to herein as compound 55A), which shows a marked preference for the inhibition of the NRG driven cells (FIG. 3D).

D. The Cellular Activity of Compound 2 Results from HER2 Inhibition

The specificity of compound 2 for the NRG-driven 2YF/3wt cells over Axl+ or 2YF/3wt cells in the presence of IL-3 indicated that the compound was likely interfering with signaling at the RTK level. This was confirmed by in vitro profiling against a panel of kinases, with compound 2 only showing potent inhibition of EGFR and Ab1 (Table 2).

TABLE 2
Complete profiling of compound 2
at 100 nM conducted by Nanosyn.

| Kinase | % Inhibition | | |
|---|---|---|---|
| EGFR | 95.595 | CDK2 | 4.145 |
| ABL1 | 95.195 | PIM-1-KINASE | 4.075 |
| LCK | 74.16 | DYRK1B | 4.01 |
| SRC | 63.24 | MST1 | 3.855 |
| FLT-3 | 59.99 | PYK2 | 3.805 |
| BLK | 59.895 | CK1-EPSILON | 3.45 |
| FGFR1 | 48.3 | EPH-B4 | 3.44 |
| MAP4K4 | 36.53 | TYRO3 | 3.29 |
| KDR | 27.32 | AMP-A1B1G1 | 2.685 |
| GSK-3-BETA | 20.465 | EPH-A2 | 2.42 |
| JAK3 | 20.43 | TRKA | 2.385 |
| TRKC | 15.655 | MET | 2.045 |
| SGK1 | 14.44 | IKK-BETA | 1.915 |
| AKT1 | 14.27 | NEK2 | 1.53 |
| IGF1R | 13.315 | IRAK4 | 1.465 |
| TIE2 | 12.835 | ITK | 1.24 |
| PDGFR-ALPHA | 11.85 | PKC-ALPHA | 1.24 |
| AURORA-A | 9.945 | JNK2 | 1.075 |
| P70S6K1 | 8.875 | CHEK1 | 0.965 |
| CSK | 6.955 | TSSK1 | 0.665 |
| P38-ALPHA | 6.605 | ROCK1 | 0.155 |
| ALK | 6.145 | MAPKAPK-2 | 0.12 |
| PRKD2 | 5.465 | PAK2 | 0.085 |
| DYRK1A | 5 | DAPK1 | −0.52 |
| PDK1 | 4.58 | SYK | −2.115 |

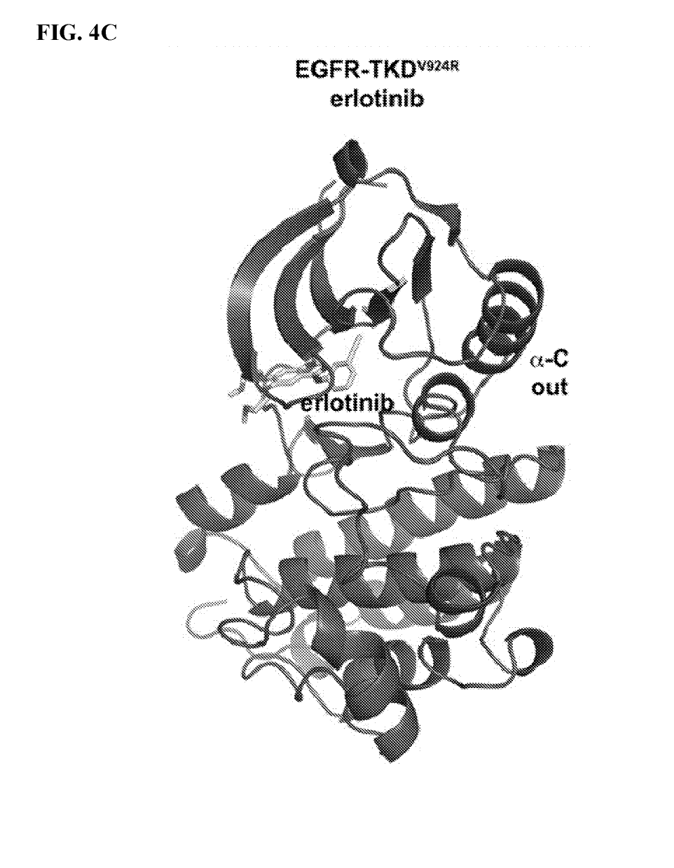

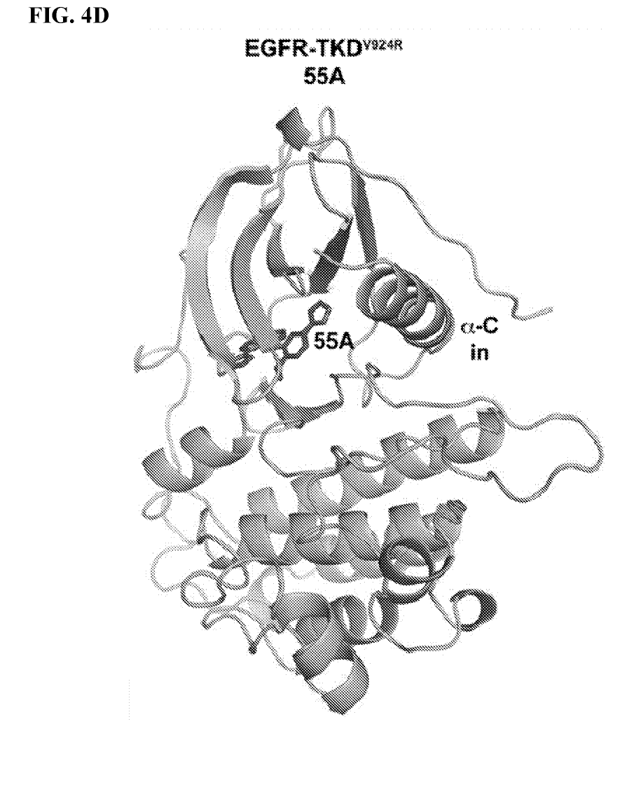

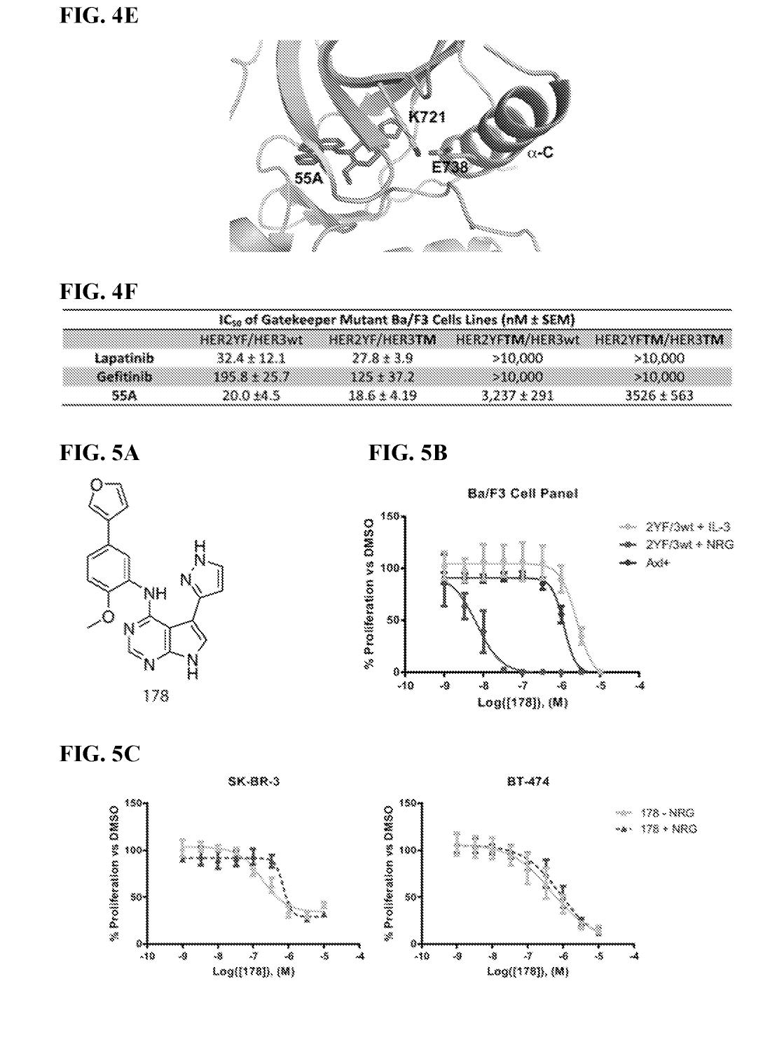

To determine the mechanism of action of compound 2, we evaluated its ability to interact with HER2 or HER3 using an in vitro kinase assay or thermofluor respectively. In embodiments, compound 2 was equipotent to lapatinib against HER2 in vitro (FIG. 4A) and, surprisingly, was also capable of binding to HER3 (FIG. 4B). Moreover, compound 2 was also able to inhibit the small amount of HER3 auto-phosphorylation seen when the purified HER3 intracellular domain is clustered, indicating that it binds to the HER3 active site (FIG. 11).

To determine how compound 2 is able to interact with multiple members of the EGFR family, we determined the X-ray crystal structure of compound 2 bound to the EGFR kinase domain (FIG. 4D, Table 3).

TABLE 3
Data collection and refinement statistics (molecular replacement).

| | EGFR/2 | |
|---|---|---|
| Data collection¹ | | |
| Space group | P 3₁ 2 1 | |
| Cell dimensions | | |
| a, b, c (Å) | 69.4, 69.4, 192 | |
| α, β, γ (°) | 90, 90, 120 | |
| Resolution (Å) | 50-3.30 | |
| R_{sym}² | 0.057 | (0.982)³ |
| I/σ | 21.4 | (1.26) |
| Completeness (%) | 99.7 | (99.7) |
| Redundancy | 3.4 | (3.5) |

| Refinement | |
|---|---|
| Resolution (Å) | 50-3.30 |
| No. reflections | 8562 |
| R_{work}/R_{free} | 0.24/0.27 |
| No. atoms | |
| Protein | 2333 |
| Ligand | 38 |
| B-factors | |
| Protein | 154.5 |
| Ligand | 128.5 |
| R.m.s. deviations | |
| Bond lengths (Å) | 0.003 |
| Bond angles (°) | 0.630 |

¹Each dataset was collected from a single crystal;
²Values for highest resolution shell are shown in parenthesis;
³CC^{1/2} is 0.562 on highest resolution shell.

Although crystals were obtained with both the wild-type kinase domain and a V924R-mutated variant, the latter were optimized most readily. The V924R kinase domain crystallizes in the inactive (autoinhibited) conformation in the absence of inhibitor or even when bound to the type I EGFR inhibitor erlotinib, because this mutation places a polar arginine side-chain in the middle of the hydrophobic patch used to form the asymmetric dimer required for EGFR to act as an activator[33, 34] (FIG. 4D). Strikingly, compound 2 was able to stabilize the active conformation of this mutated EGFR kinase domain in crystals, as evidenced by the “in conformation” of the α-C helix, which allows formation of the characteristic salt-bridge between the P3 lysine and the α-C glutamate, as well as the ordered extension of the activation loop (FIGS. 4D-4E). This finding indicates that this scaffold has a strong preference for binding and stabilizing the active conformation of EGFR family kinase domains. Interestingly, the HER3 kinase domain has only ever been crystallized in the inactive conformation, and failed to crystallize after introducing mutations designed to destabilize the inactive state or in the presence of compound 2 [35, 36]. This suggests that compound 2 may be stabilizing an alternate HER3 kinase domain conformation that could potentially prevent its ability to allosterically activate HER2.

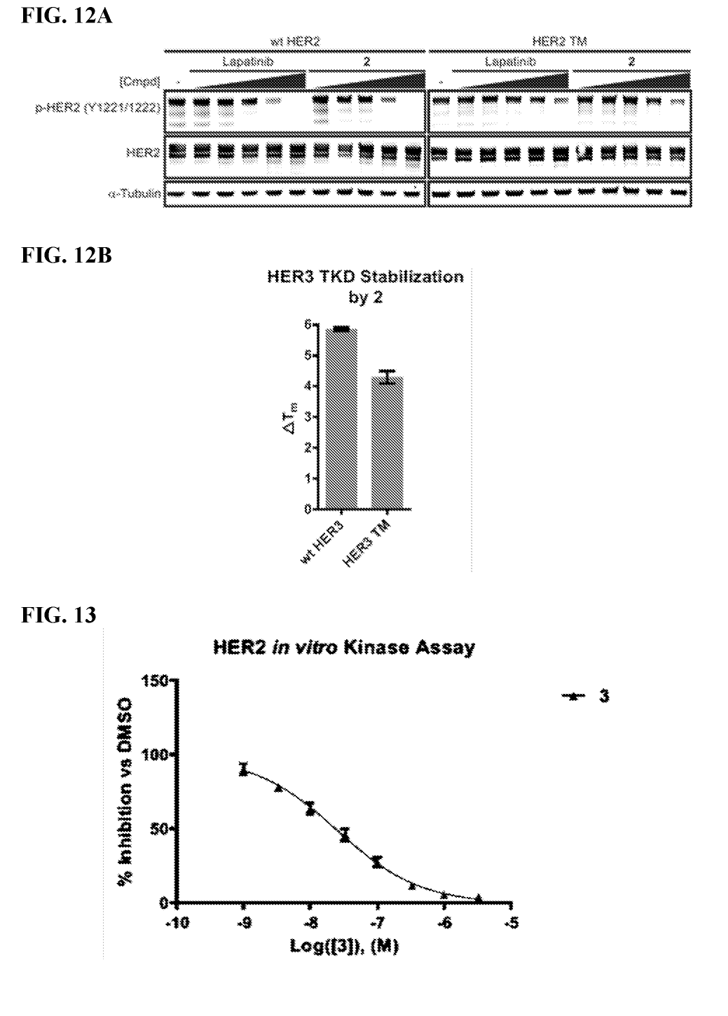

In order to determine whether binding to HER2, HER3, or to both, is responsible for the anti-proliferative activity of compound 2 we created a series of Ba/F3 cell lines dependent on NRG-induced HER2/HER3 heterodimers that possessed methionine gatekeeper mutations (TM) in either kinase (2YF/3TM and 2YFTM/HER3wt) or both (2YFTM/3TM). The methionine gatekeeper mutation has been shown to prevent lapatinib from binding to HER2, and was able to reduce the ability of compound 2 to bind to either kinase in isolation (FIGS. 12A-12B).

Both lapatinib and gefitinib (an EGFR inhibitor capable of inhibiting HER2 to a lesser extent) were unable to inhibit the proliferation of either Ba/F3 cell line that contained the gatekeeper mutation in HER2 (FIG. 4F). Similarly, inhibition by compound 2 was only affected by the HER2 gatekeeper mutation, whereas the gatekeeper mutation in HER3 had little influence (FIG. 4F). These data indicate that the cellular activity of compound 2 is due to inhibition of HER2 kinase activity.

E. Type I Inhibitors of HER2 are Insensitive to NRG Mediated Resistance

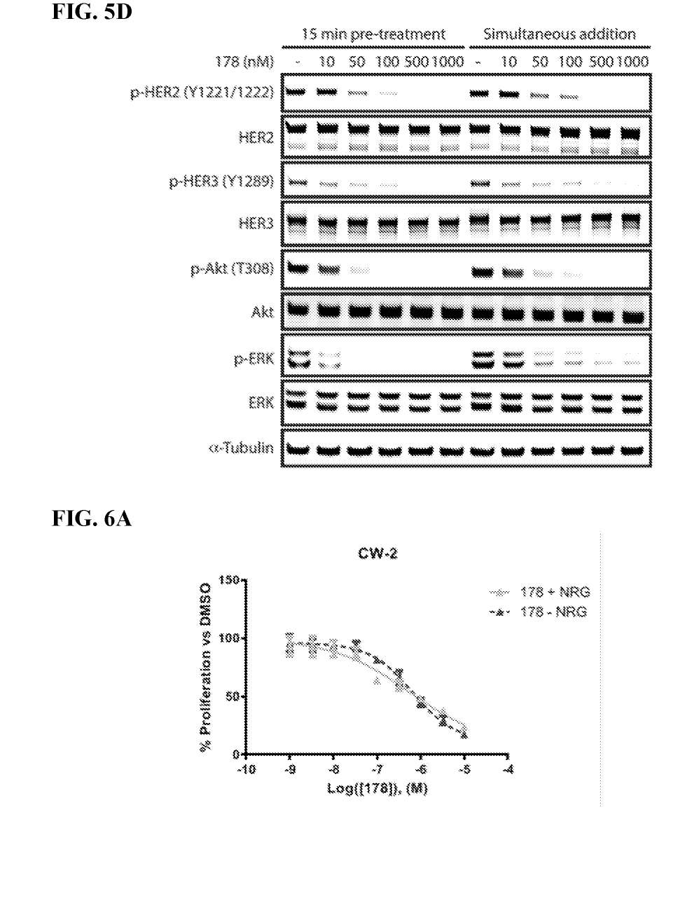

Further experimentation was performed to optimize the potency of these inhibitors against the Ba/F3 cell line panel. The crystal structure suggested that the extra-cyclic NH linker could form an intramolecular hydrogen bond with the 2-furan, which would help to stabilize the inhibitor in a conformation necessary for binding to the active kinase. The structure also suggested that limiting the charge density on the other 2-furan ring would prevent negative interactions with the kinase. With these parameters in mind, the second optimization effort led to compound 3 (also referred to herein as compound 178), which showed superior activity to lapatinib in the 2YF/3wt+NRG cells, and specificity for this cell line over the others by multiple orders of magnitude (FIGS. 5A-5B, Table 4).

TABLE 4
Table of IC₅₀ values (nM ± S.D., n = 3 for 2YF/3wt + IL-3, Axl, and 2YF/3wt + NRG.
n = 2 for 2YF-L755S/3wt + NRG, 2YF-YVMA/3wt + NRG, and 2YF-VC/3wt + NRG).

| | | | | 2YF-L755S/ | 2YF-YVMA/ | 2YF-VC/ |
|---|---|---|---|---|---|---|
| | 2YF/3wt + IL-3 | Axl | 2YF/3wt + NRG | 3wt + NRG | 3wt + NRG | 3wt + NRG |
| Lapatinib | >3,333 | >3,333 | 63.6 ± 12.5 | >3,333 | >10,000 | >3,333 |
| TAK-285 | >10,000 | >3,333 | 398.6 ± 110.7 | NT | NT | NT |
| Compound 3 | 2536.7 ± 217.2 | 1172.3 ± 106.0 | 6.8 ± 0.2 | 38.2 ± 2.9 | 11.4 ± 0.1 | 7.7 ± 1.0 |

In vitro kinase profiling of compound 3 revealed a similar profile to compound 2 as well as similar potency against HER2 (Table 5 and Table 6, and FIG. 13).

TABLE 5
Complete profiling of compound 2 at 1 μM conducted by Nanosyn.

| Kinase | % Inhibition | Kinase | % Inhibition |
|---|---|---|---|
| ABL1 | 97.22 | PYK2 | 20.195 |
| EGFR | 96.945 | MST1 | 19.93 |
| LCK | 93.57 | ALK | 19.825 |
| BLK | 90.405 | PDK1 | 18.29 |
| FLT-3 | 89.42 | CDK2 | 17.555 |
| SRC | 88.935 | DYRK1A | 16.68 |
| FGFR1 | 86.27 | ITK | 13.945 |
| MAP4K4 | 78.705 | AMP-A1B1G1 | 12.91 |
| KDR | 71.28 | P38-ALPHA | 12.91 |
| JAK3 | 69.355 | MET | 10.14 |
| GSK-3-BETA | 68.82 | PIM-1-KINASE | 8.705 |
| TIE2 | 59.745 | JNK2 | 6.62 |
| SGK1 | 53.445 | PKC-ALPHA | 5.57 |
| IGF1R | 53.275 | TYRO3 | 5.56 |
| PDGFR-ALPHA | 52.5 | TRKA | 4.495 |
| AURORA-A | 47.18 | NEK2 | 2.985 |
| TRKC | 46.91 | IKK-BETA | 2.91 |
| P70S6K1 | 38.41 | CHEK1 | 2.265 |
| AKT1 | 33.05 | DAPK1 | 1.425 |
| PRKD2 | 26.63 | IRAK4 | 1.42 |
| CSK | 24.82 | ROCK1 | 1.16 |
| CK1-EPSILON | 24.12 | PAK2 | 1.07 |
| DYRK1B | 22.625 | MAPKAPK-2 | 0.575 |
| EPH-B4 | 21.67 | TSSK1 | −3.06 |
| EPH-A2 | 20.97 | SYK | −6.41 |

TABLE 6
Complete profiling of compound 3 at 1 μM conducted by Nanosyn.

| Kinase | % inhibition | Kinase | % inhibition |
|---|---|---|---|
| MAP4K4 | 100 | ITK | 24 |
| EGFR | 99 | AMP-A1B1G1 | 20 |
| ABL1 | 98 | PRKACA | 20 |
| LCK | 98 | CDK1 | 18 |
| SRC | 95 | MET | 16 |
| FGFR1 | 93 | ROCK1 | 16 |
| MST1 | 84 | CK1-EPSILON | 11 |
| MST2 | 84 | CDK2 | 10 |
| JAK2 | 83 | CHEK1 | 10 |
| FLT-3 | 78 | IKK-BETA | 8 |
| KDR | 74 | JNK2 | 7 |
| MEK2 | 70 | PIM-1-KINASE | 7 |
| MEK1 | 66 | PI3K-ALPHA | 6 |
| PDGFR-ALPHA | 66 | PDK1 | 5 |
| CSK | 62 | PAK2 | 3 |
| JAK1 | 52 | P38-ALPHA | 2 |
| TYK2 | 51 | CAMK4 | 0 |
| IGF1R | 46 | DAPK1 | −1 |
| DYRK1A | 41 | MAPK1 | −2 |
| PYK2 | 41 | MAPKAPK-2 | −6 |
| GSK-3-BETA | 40 | NEK2 | −9 |
| SYK | 35 | FAK | −10 |
| IRAK4 | 34 | | |
| AURORA-A | 31 | | |
| SGK1 | 31 | | |
| PKC-ALPHA | 30 | | |
| EPH-A2 | 28 | | |
| PRKD2 | 28 | | |

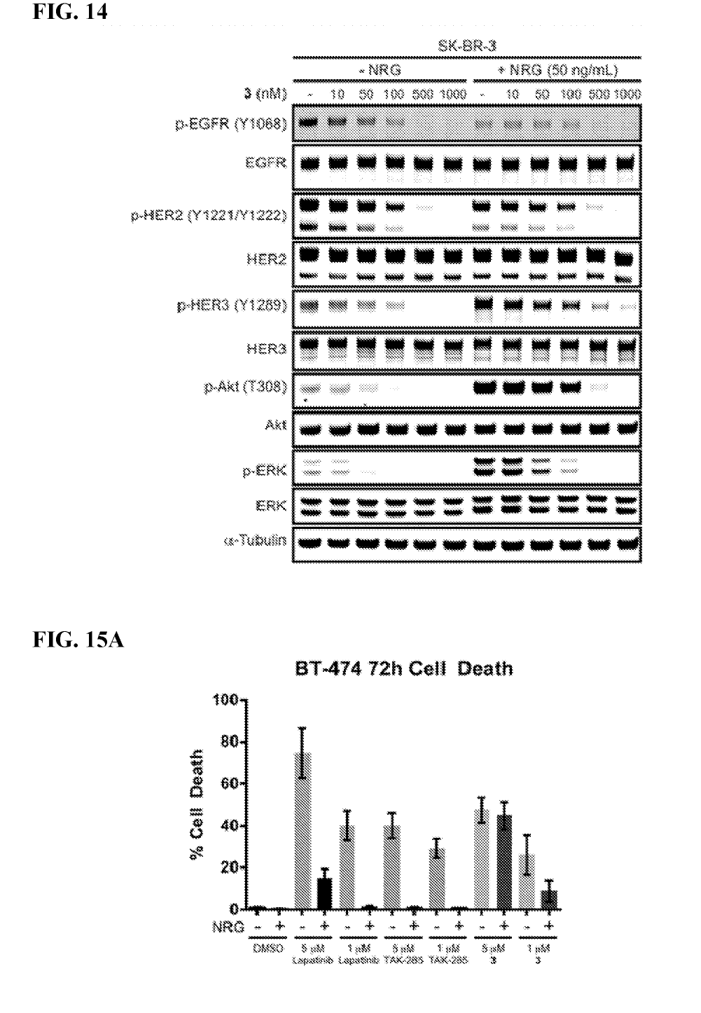

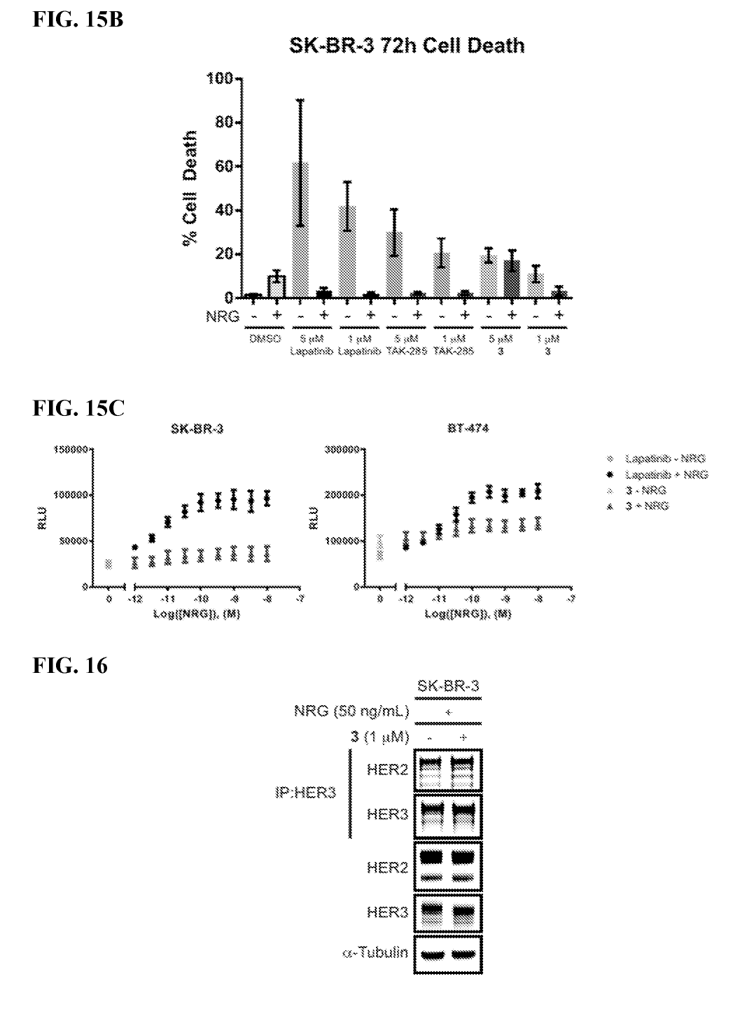

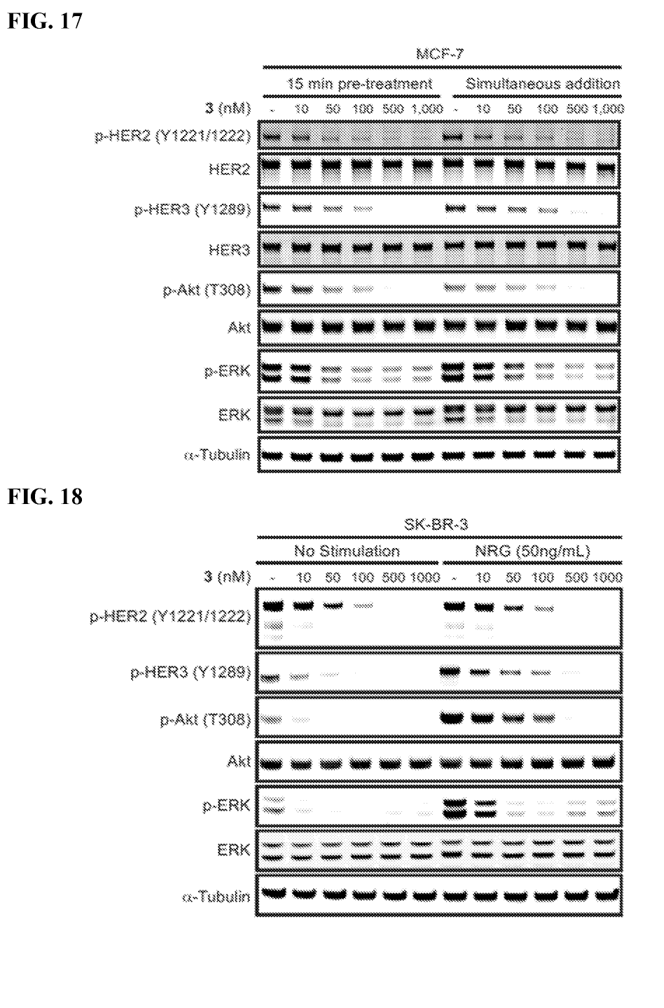

Compound 3 showed little to no shift in its ability to inhibit the growth or signaling of HER2 over-expressing cell lines in the presence of NRG (FIG. 5C, and FIG. 14). Additionally, unlike lapatinib and TAK-285, 1 μM compound 3 was able to inhibit the proliferation of HER2 overexpressing cells over a dose response of NRG, and was also able to induce cell death in the presence of NRG (FIGS. 15A-15C). To confirm that compound 3 could bind to the actively signaling HER2/HER3 heterodimer we looked at signaling 15 min after the addition of NRG in SK-BR-3 cells either pre-treated with compound 3 followed by NRG stimulation or simultaneously treated with compound 3 and NRG. The minimal influence of NRG on the ability of compound 3 to inhibit all levels of signaling with or without pre-incubation, especially when compared to lapatinib (such as comparing FIG. 5D to FIG. 2A), suggests that it is fully capable of binding to and inhibiting the active HER2/HER3 complex, which were not disrupted by compound 3 (FIG. 16). A similar result was obtained in the non-HER2 amplified MCF-7 cells, and when NRG or vehicle was added prior to a dose response of compound 3 (FIG. 17 and FIG. 18).

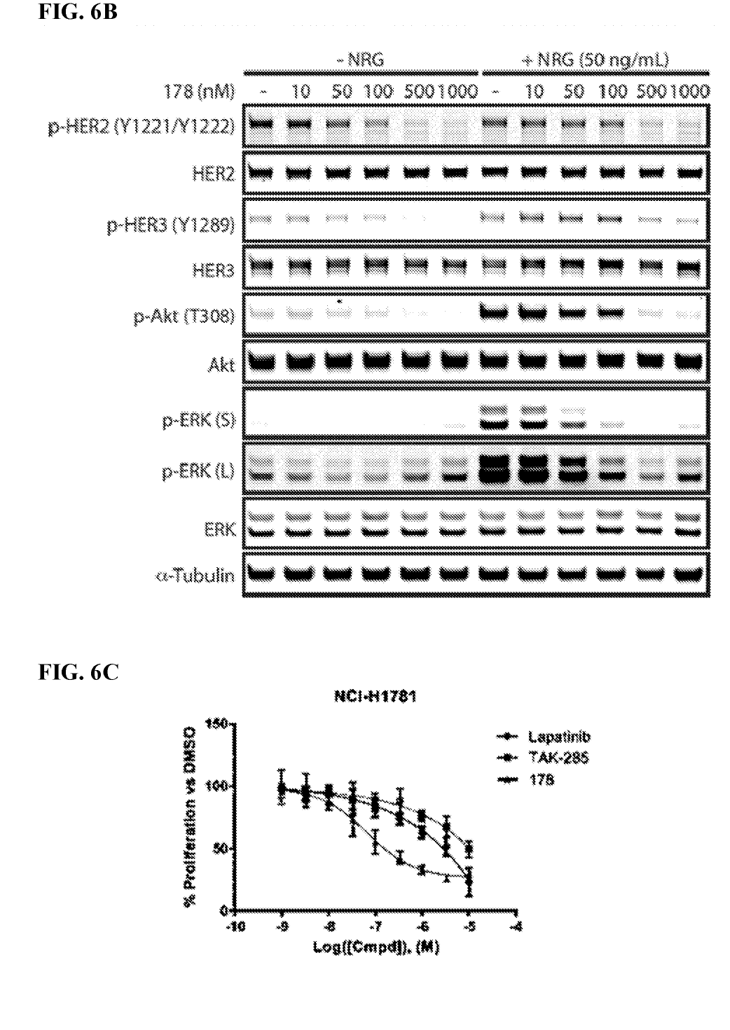

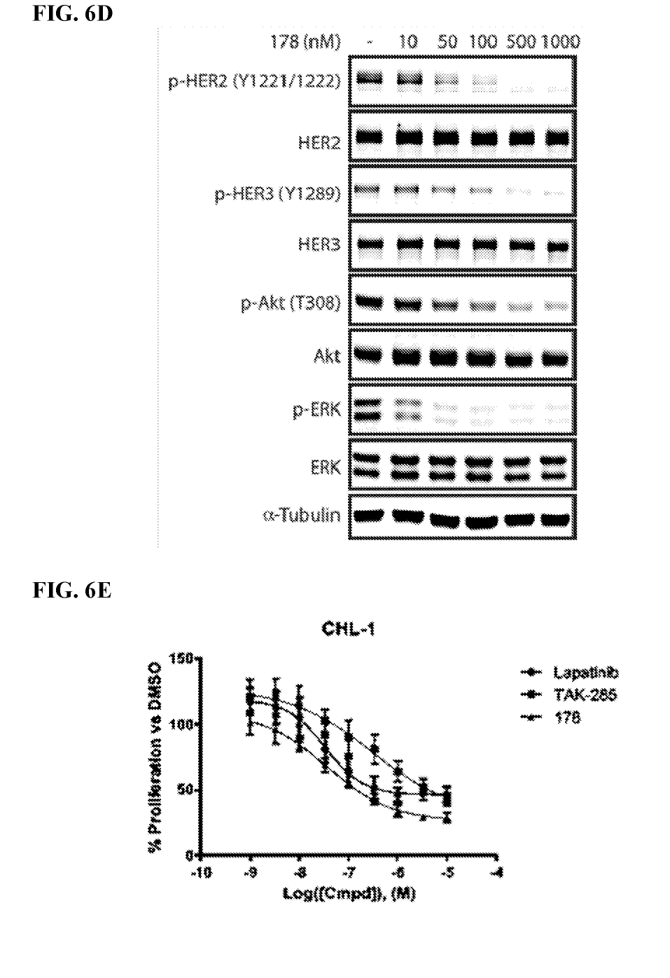

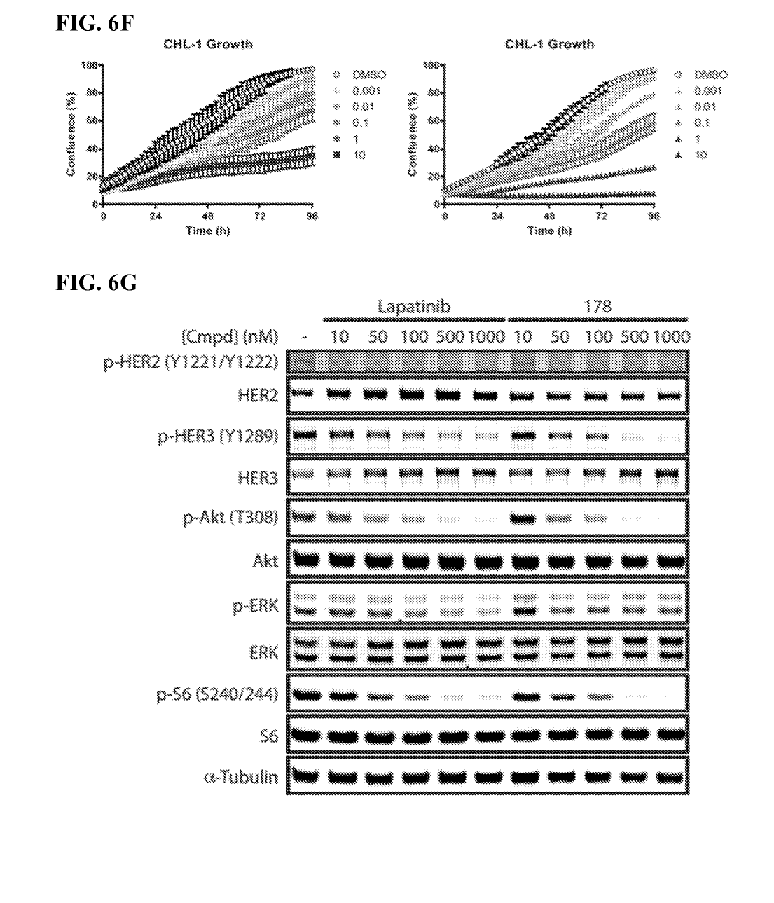

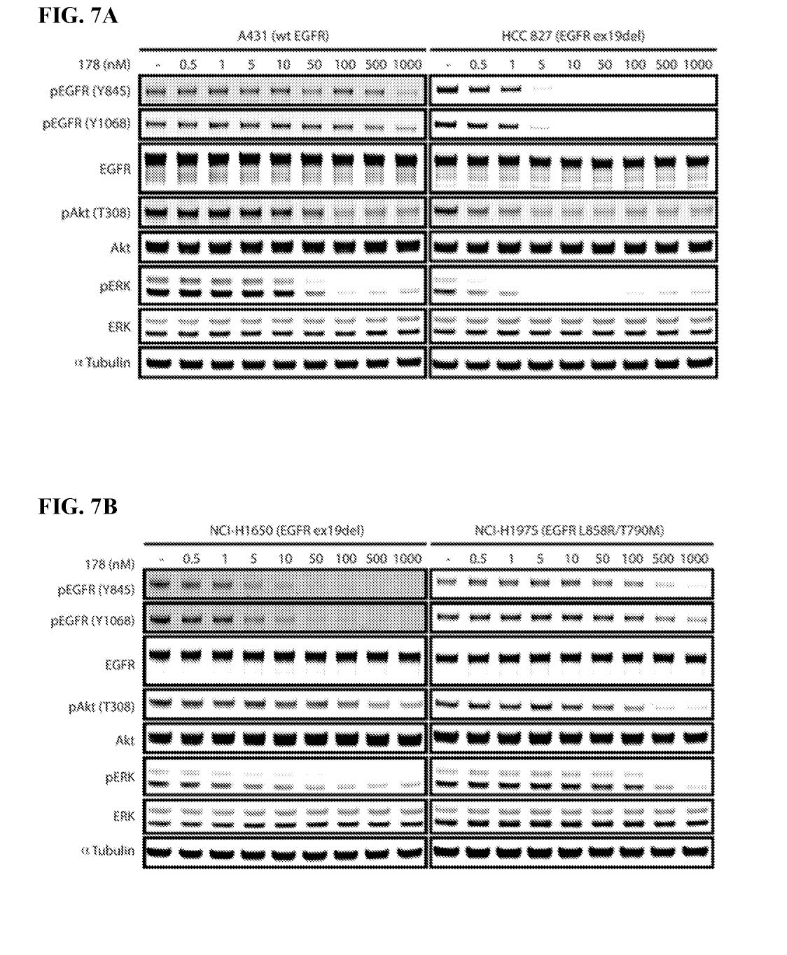

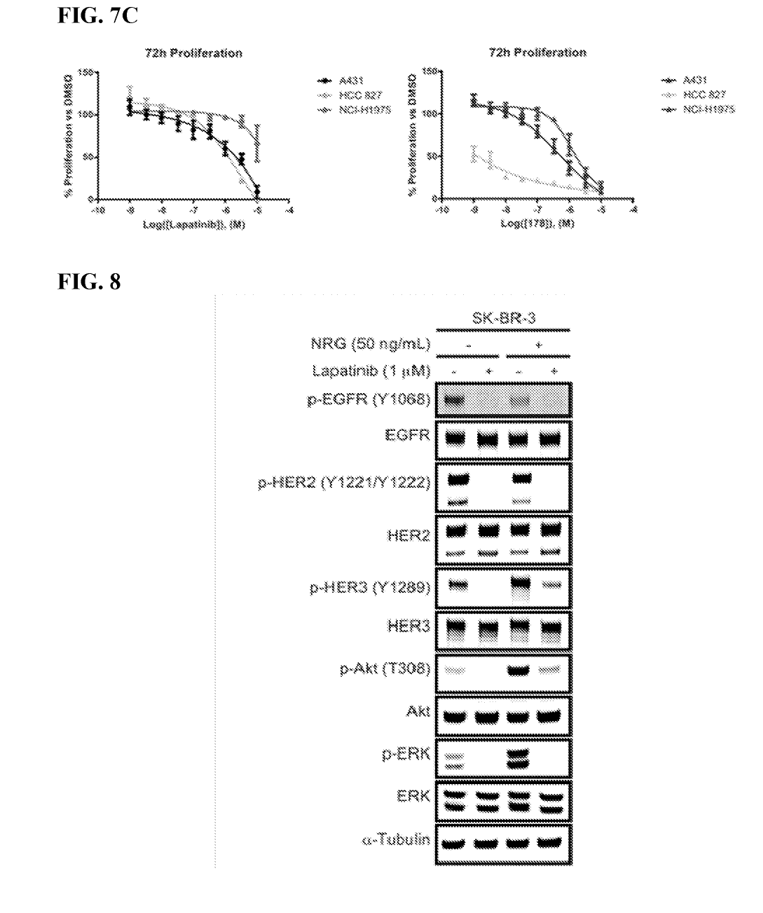

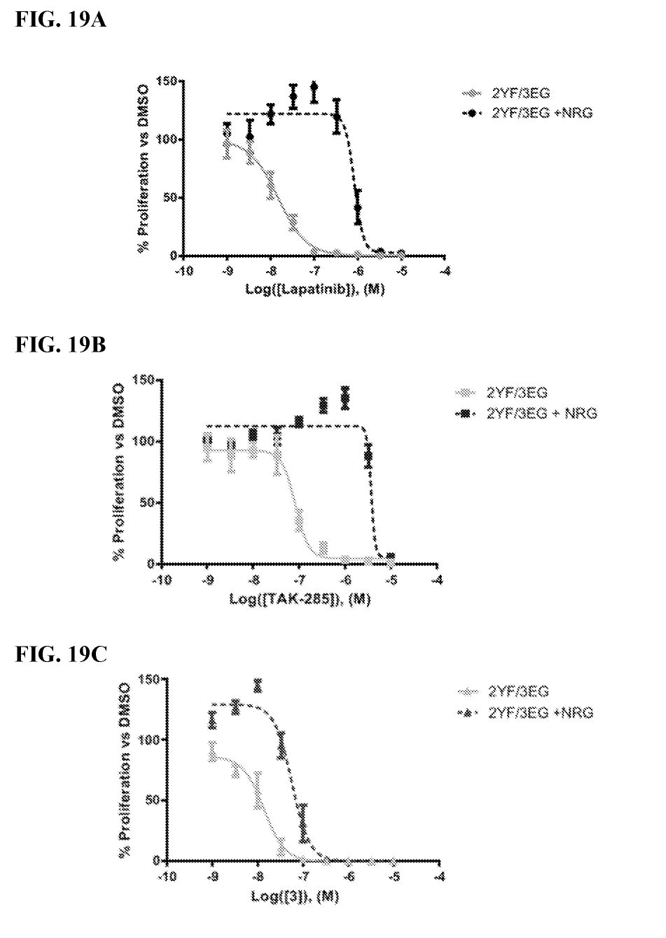

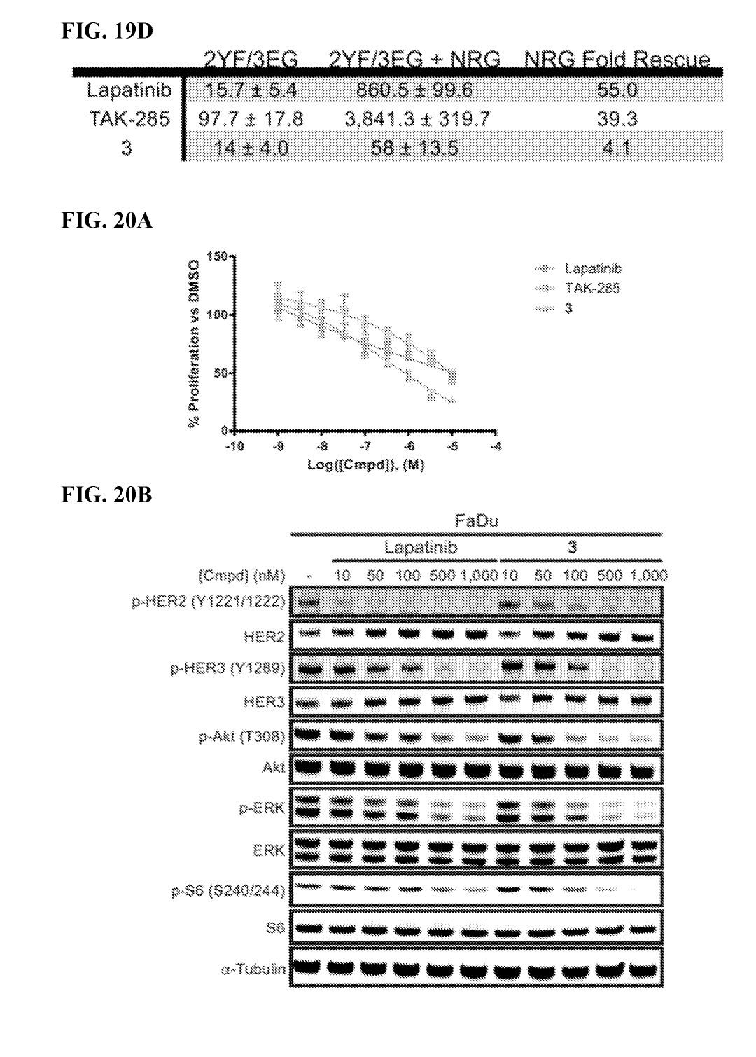

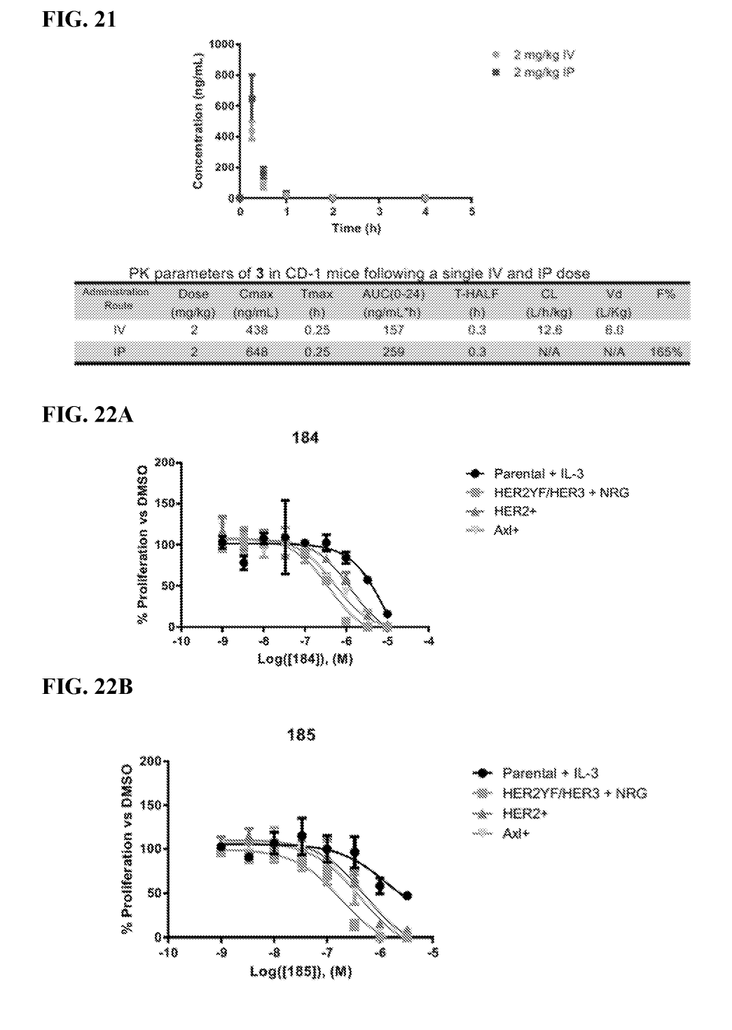

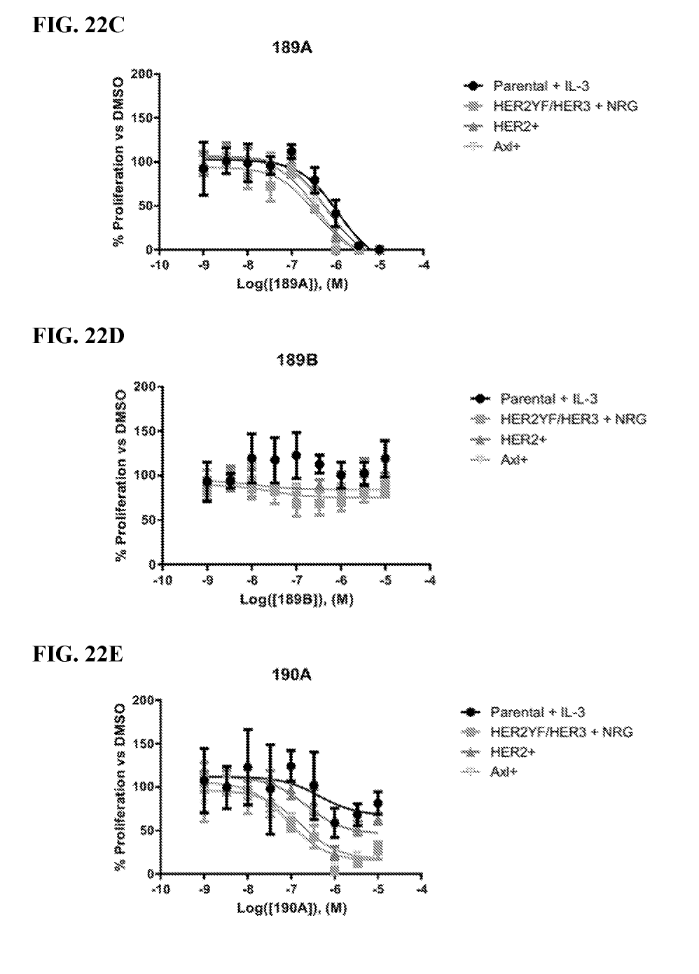

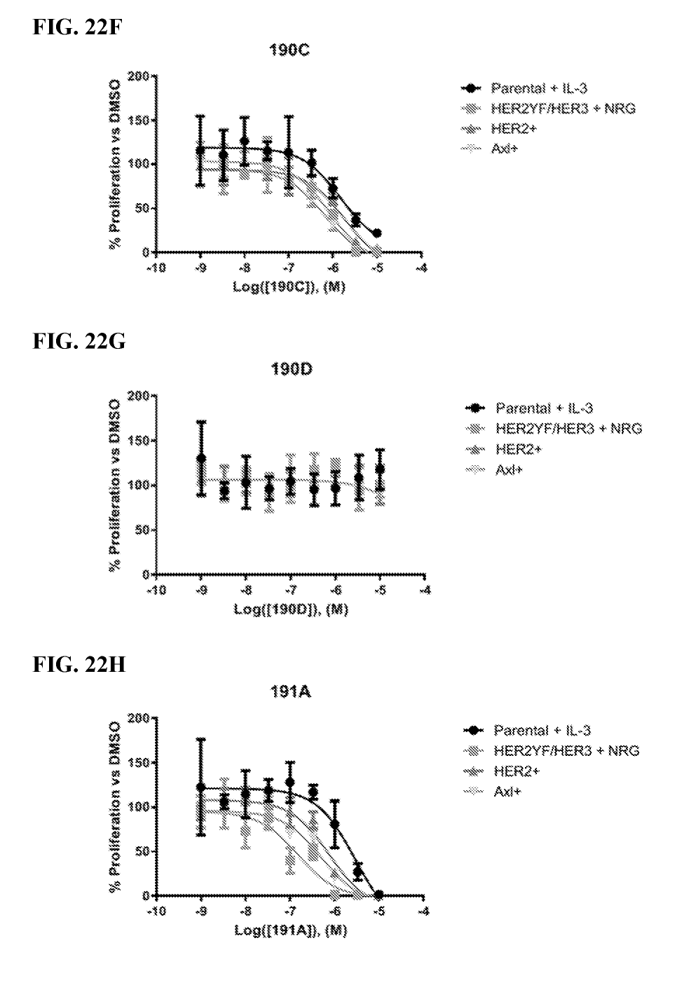

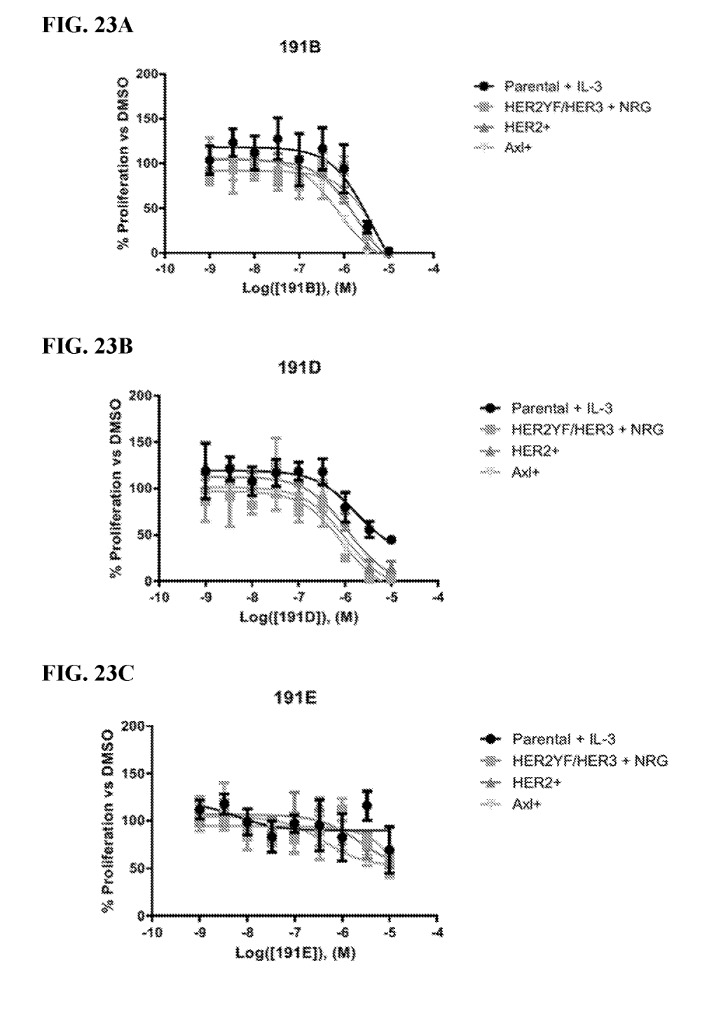

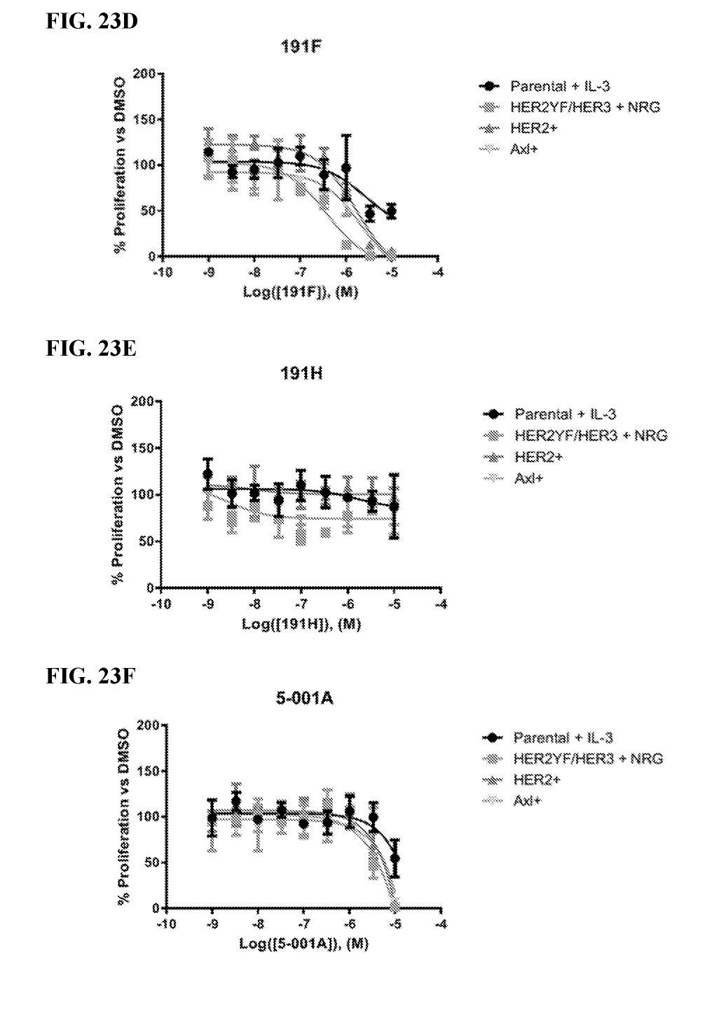

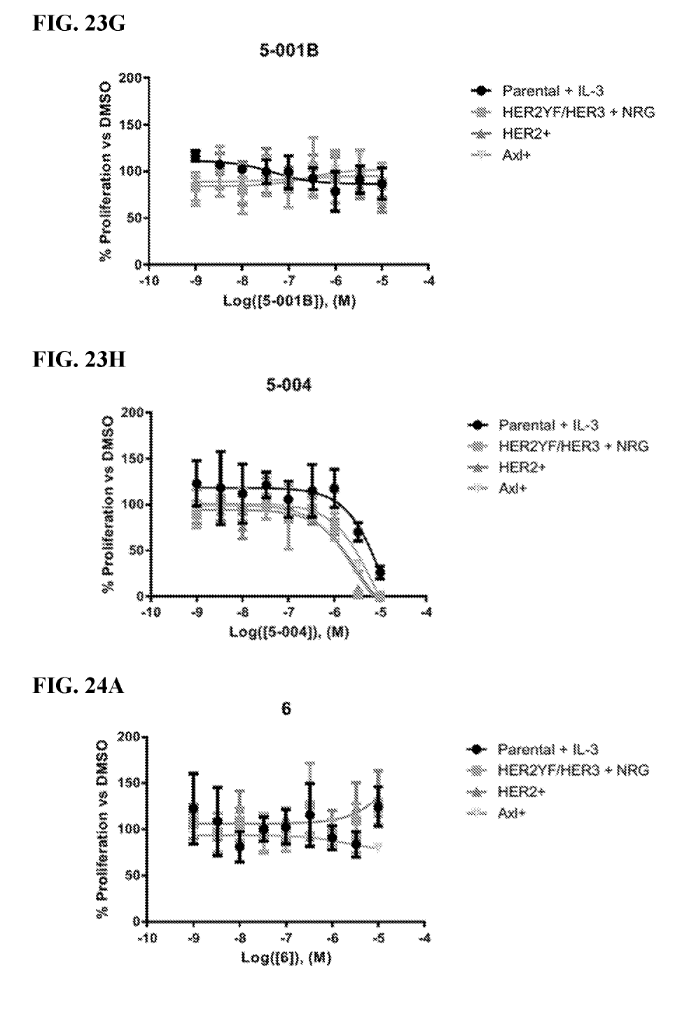

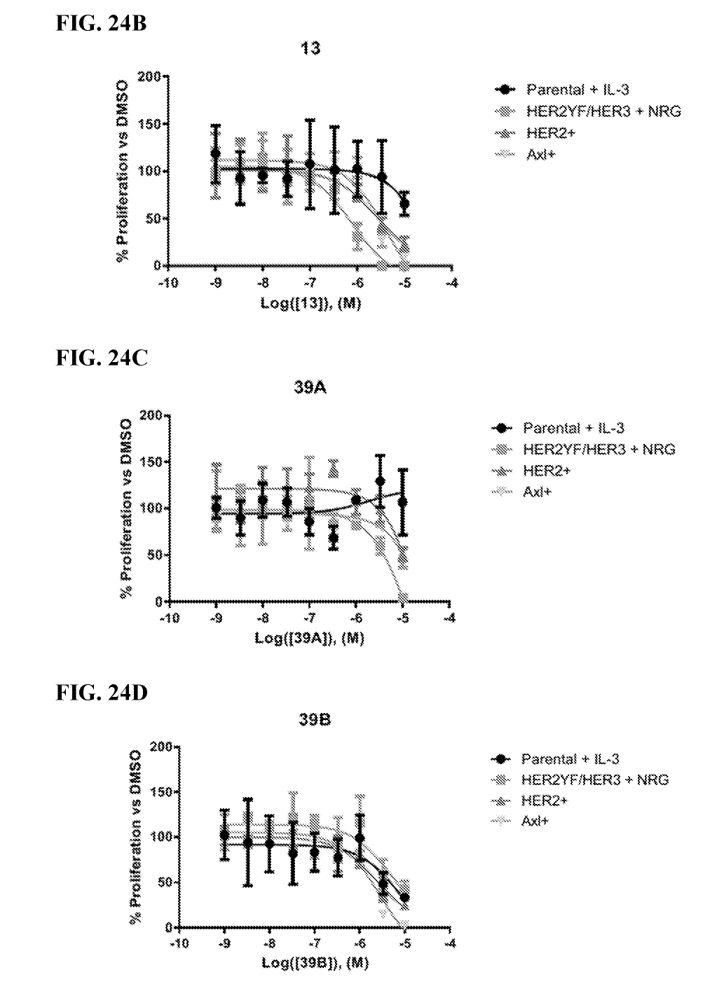

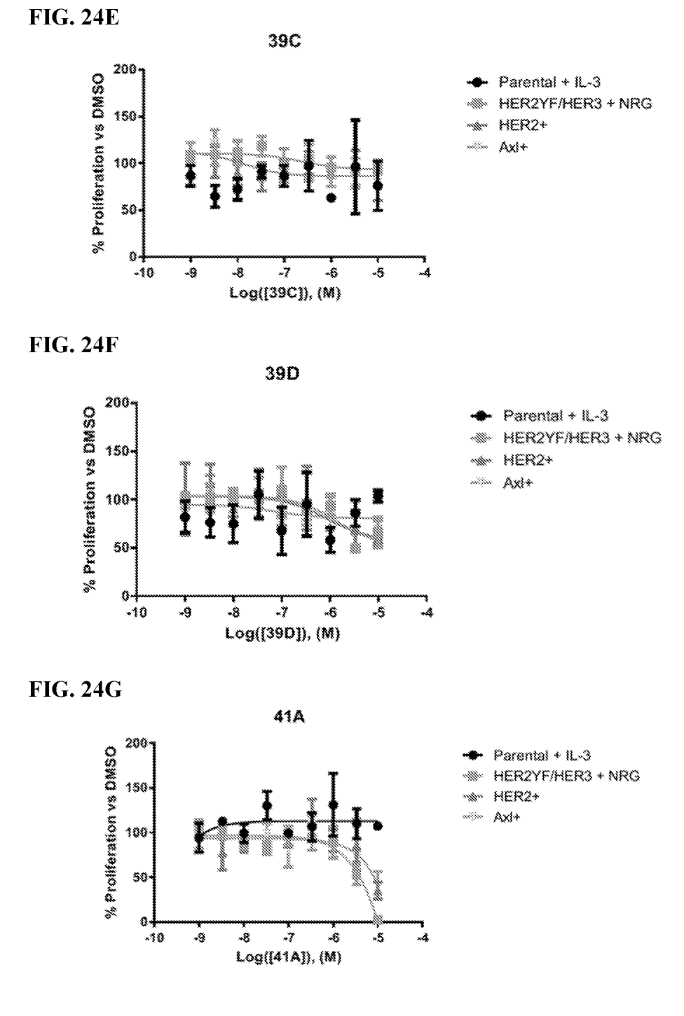

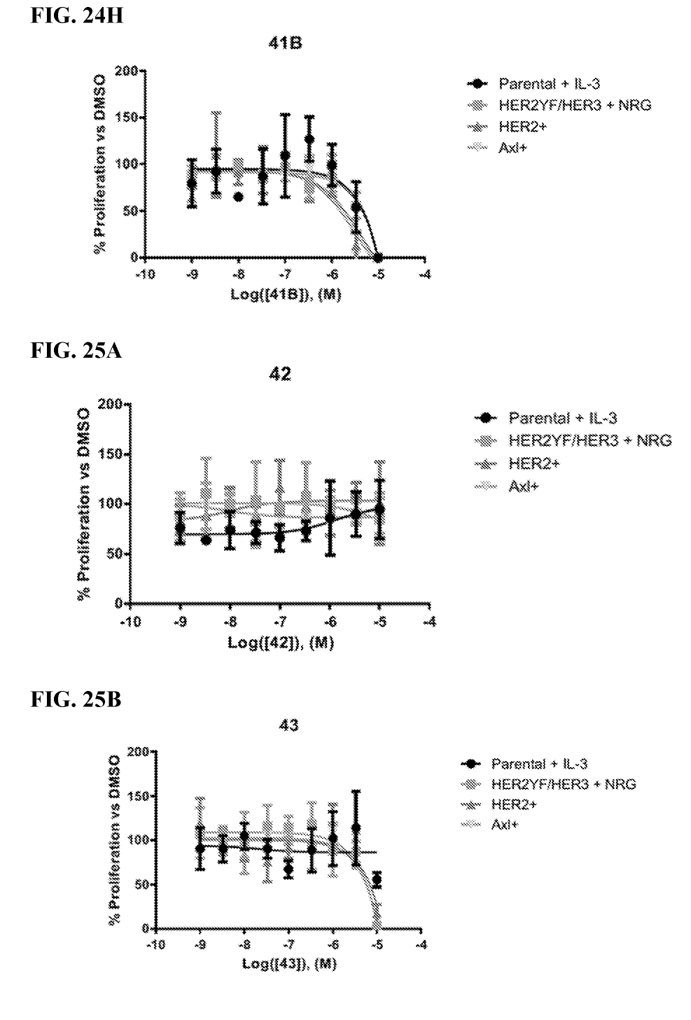

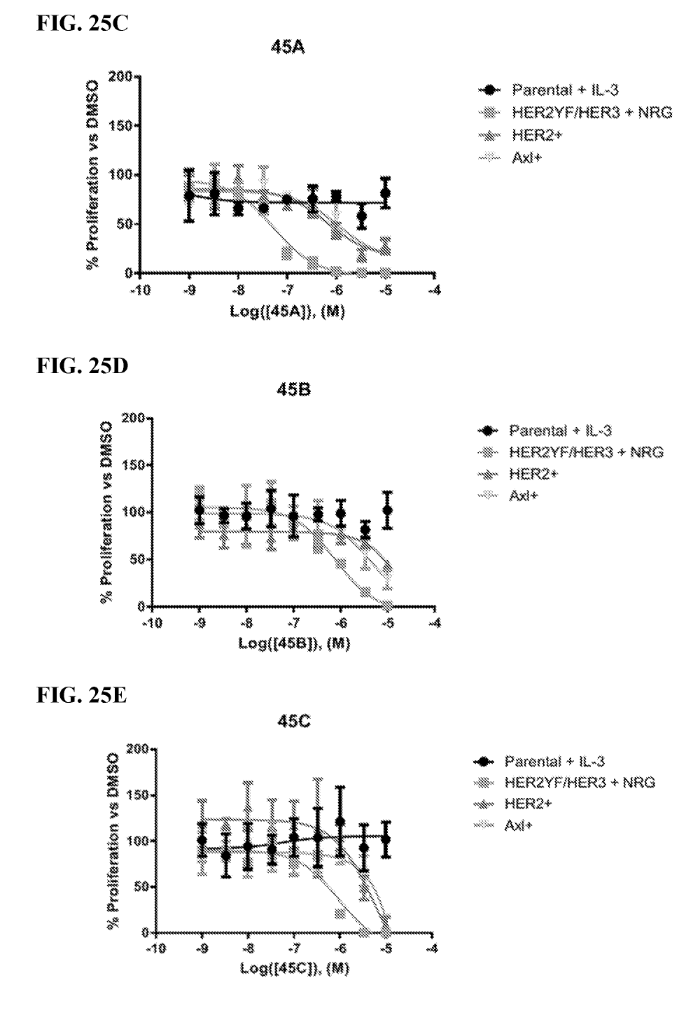

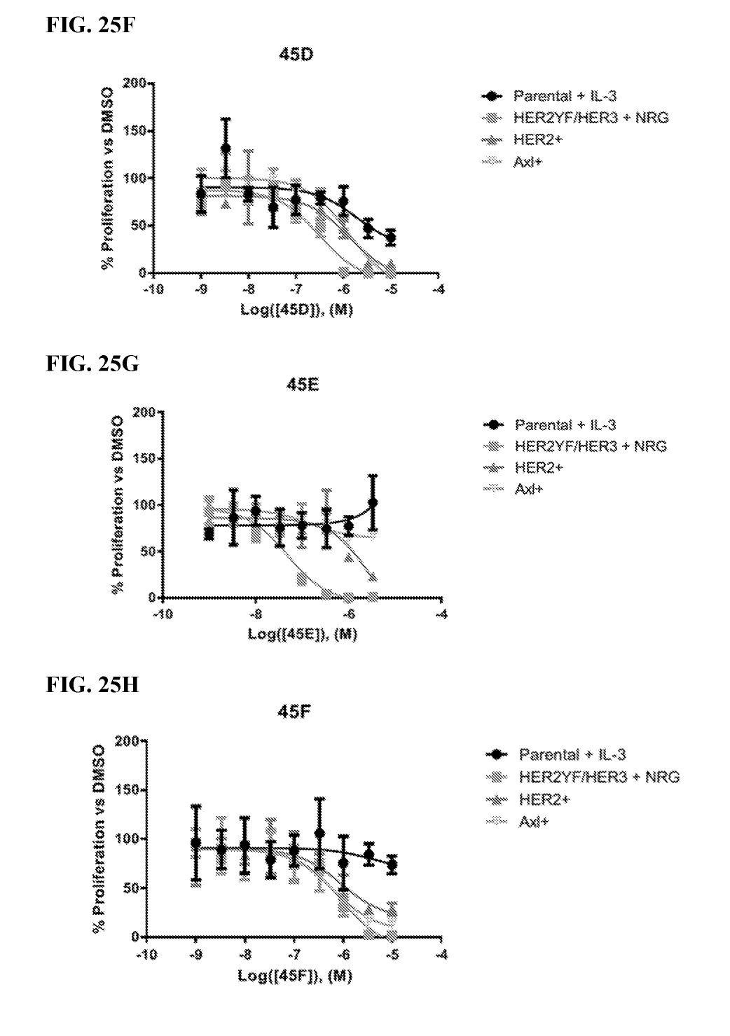

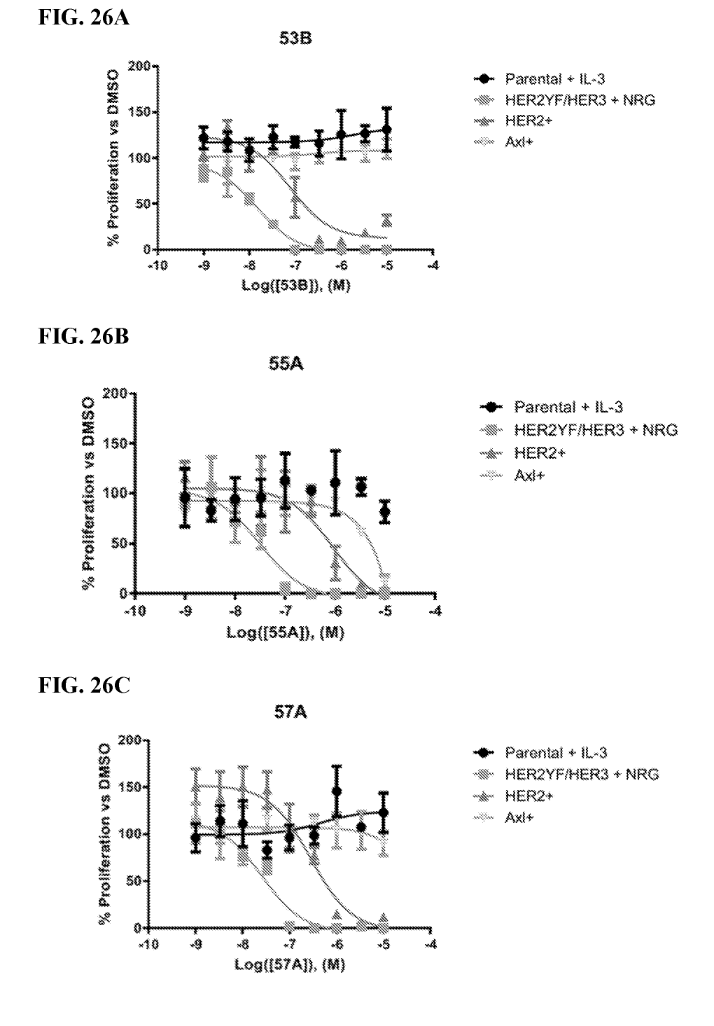

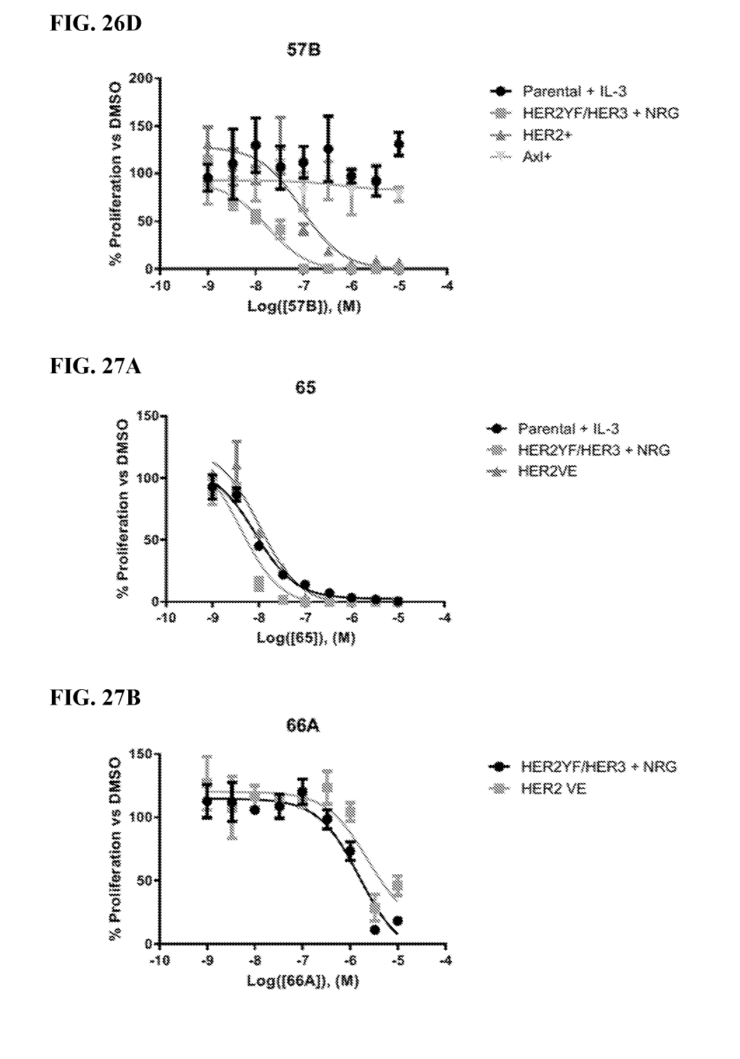

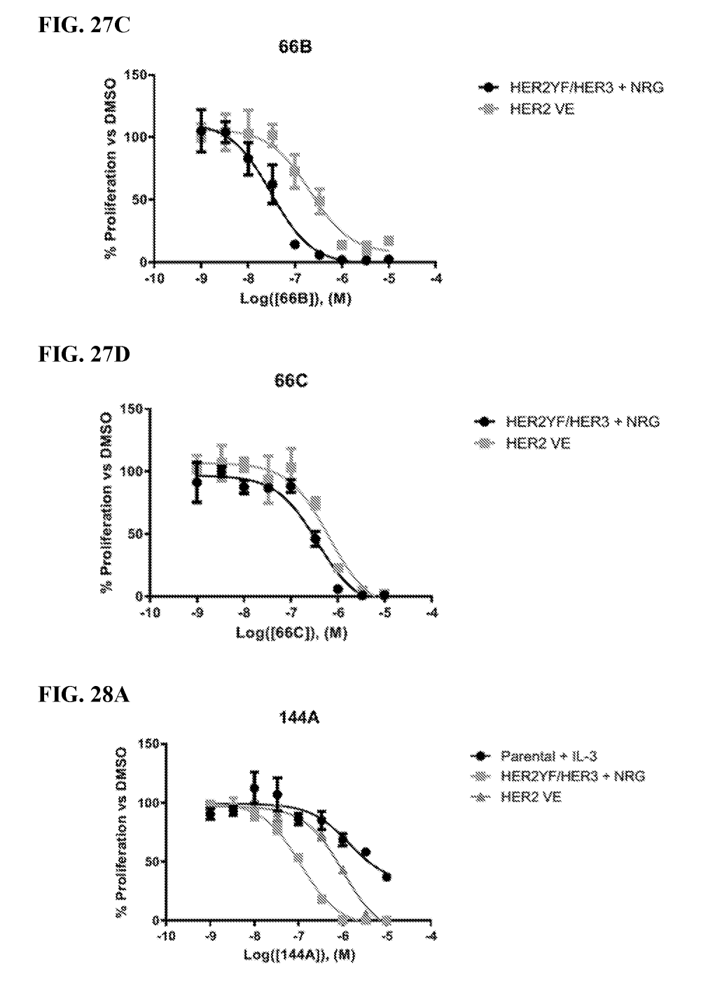

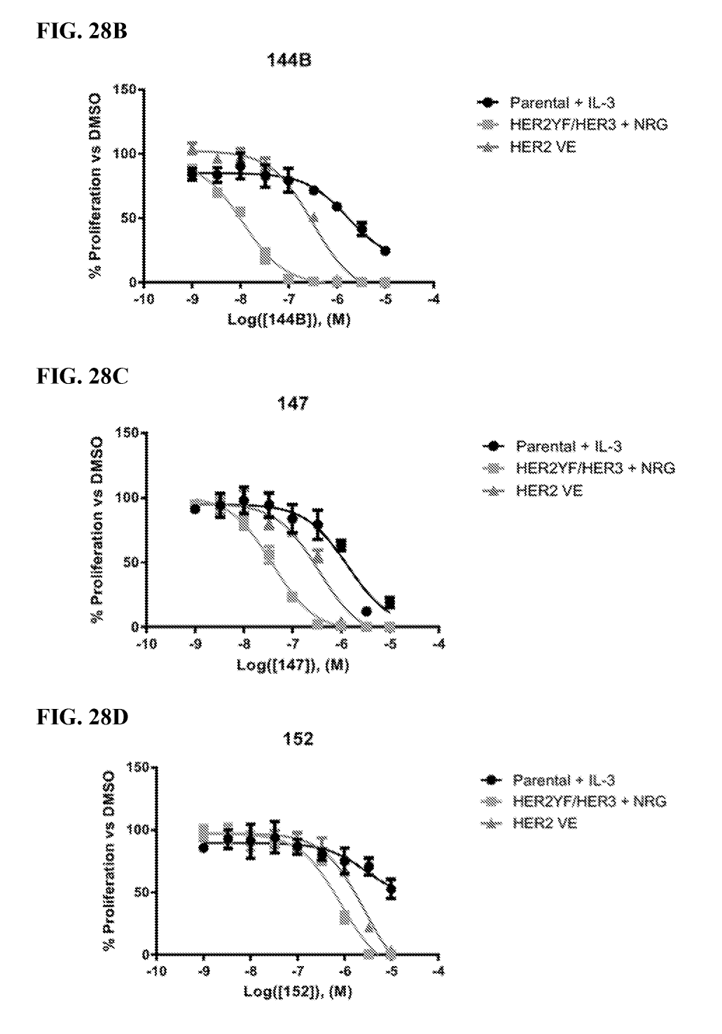

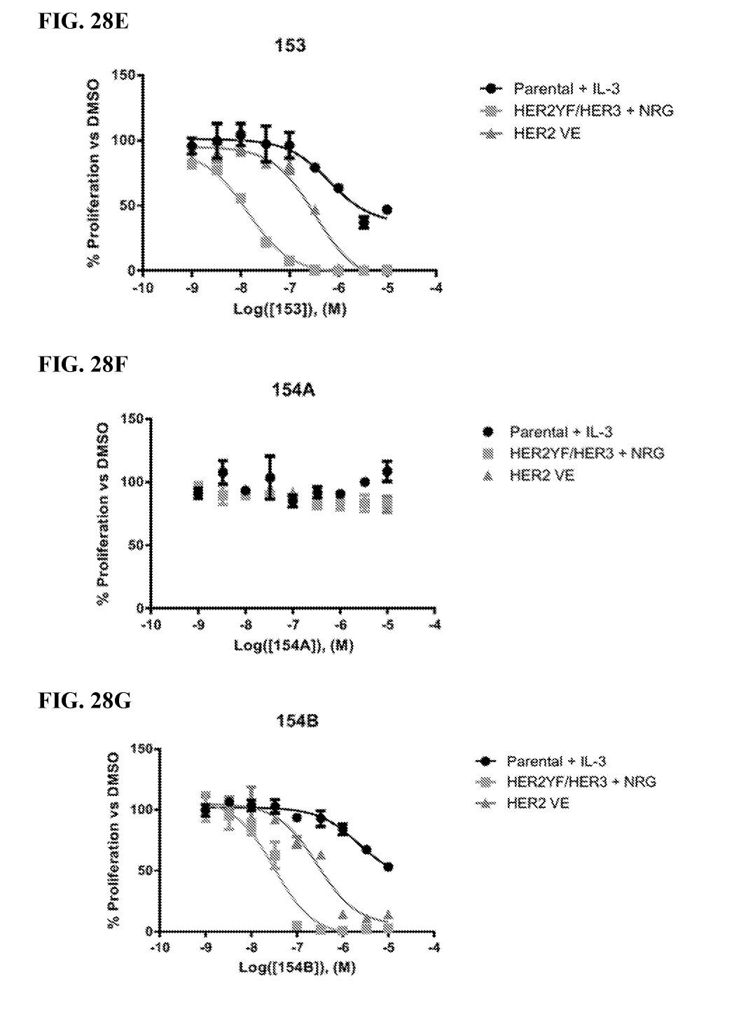

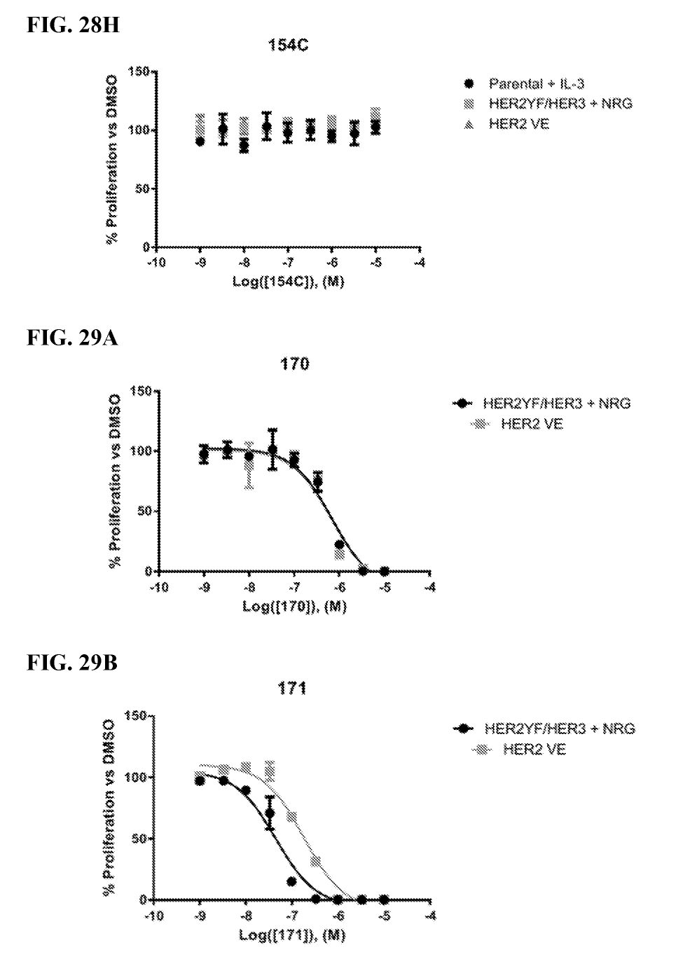

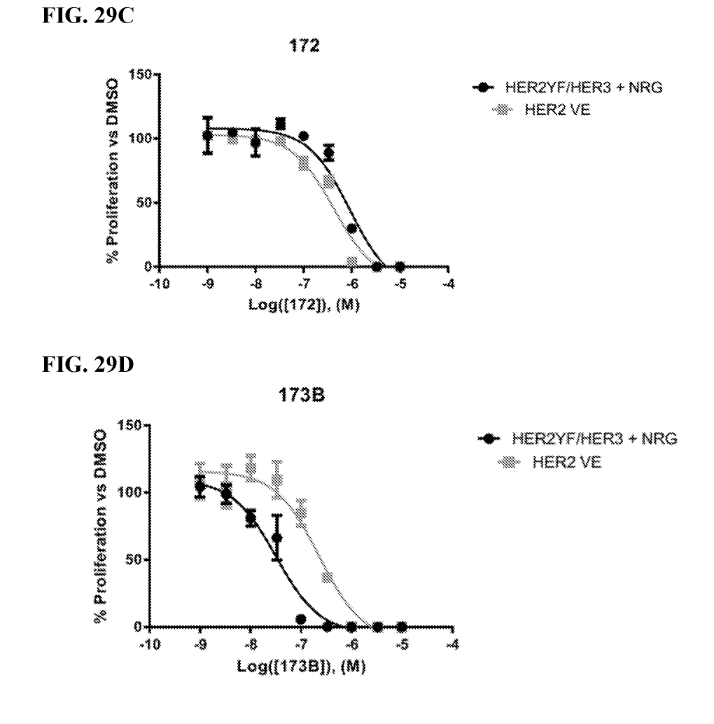

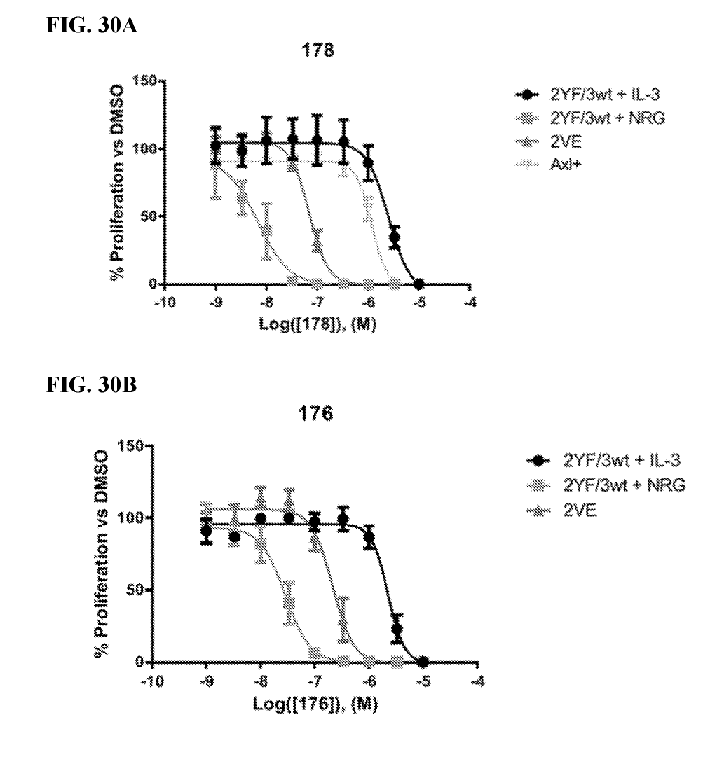

Consistent with the results in the HER2 overexpressing cell lines, CW-2 cells that harbor a HER3-activating mutation were equally sensitive to compound 3+/−NRG in both proliferation and signaling assays (FIGS. 6A-6B). This superior activity of compound 3 compared to lapatinib towards the CW-2 cells was not due solely to the L755S mutation in HER2, as a similar trend was also seen in a Ba/F3 cell line dependent on the HER3 mutant, HER2YF/HER3E928G (2YF/3EG), which can grow independently of NRG. In these cells the addition of NRG reduces lapatinib and TAK-285 sensitivity by 55 and 40 fold respectively, but affects sensitivity to compound 3 by less than 5-fold (FIGS. 19A-19D). The ability of compound 3 to inhibit the activated form of HER2 was not limited to growth factor-induced heterodimers, as the mutationally activated form of HER2 in NCI-H1781 cells was rapidly and fully inhibited by compound 3, which translated into its ability to inhibit the proliferation of this cell line (FIGS. 6C-6D). To further evaluate the potential of compound 3 against HER2 mutants within a HER2/HER3 heterodimer we transduced wt HER3 containing Ba/F3 cells with reported HER2 oncogenic mutants in the HER2YF construct[15, 16]. The resulting cell lines L755S (2YF-L755S/3wt), Y772 A775 dup (2YF-YVMA/3wt), and G776del insVC(2YF-VC/3wt) remained sensitive to compound 3 but showed complete resistance to lapatinib (Table 4).

An additional mechanism by which cancers can become dependent on HER2/HER3 dimers is through NRG mediated autocrine signaling. Proliferation of the NRG autocrine dependent CHL-1 cell line was similar between lapatinib and compound 3 when measured at 72 h using Cell Titer Glo (FIG. 6E). However, monitoring the growth of CHL-1 cells using microscopy over the same time period showed that the anti-proliferative activity of compound 3 is immediate and more potent when compared to lapatinib, which takes some time to begin exerting its weaker anti-proliferative effect (FIG. 6F).

We next examined signaling in CHL-1 cells after 24 h drug treatment and found that compound 3 was able to inhibit NRG autocrine signaling in the presence of feedback, evidenced by the increasing expression of HER2 and HER3 with increasing concentration of drug (FIG. 6G). To further examine the differing abilities of compound 3 and lapatinib to inhibit feedback-released signaling in the NRG autocrine cells, we pre-treated CHL-1 cells with lapatinib for 24 h to induce feedback signaling, washed the cells, and then treated with a dose response of either lapatinib or compound 3 for an additional 24 h. Whereas the ability of lapatinib to inhibit feedback signaling was reduced as compared to the 24 h treatment by itself, compound 3 showed little to no shift in its ability to inhibit signaling—and showed complete inhibition at 1 μM (FIG. 6H). Similar results were obtained in FaDu cells, which are also dependent on NRG autocrine signaling (FIGS. 20A-20B).

To determine the feasibility of using compound 3 in vivo we analyzed the pharmacokinetics after either IV or IP administration in mice (FIG. 14) and also reported for two compounds in Example I.

The conformational dynamics of protein kinases are critical for their function, and for many of the adaptable characteristics of kinase-driven signaling pathways such as hypersensitivity, insulation, feed-back inhibition, feed-forward activation, and desensitization. Particular kinase conformations also offer access to distinctive structural features that can be exploited in the design of inhibitors to gain selectivity even among well conserved protein families. The DFG in/α-C out binding inhibitor lapatinib targets the inactive state of a kinase with its benzyl ether substituent. What has so far been largely unappreciated is the vulnerability of type I inhibitors to mechanisms that stabilize the active state of the targeted kinases, which lead to drug resistance as we describe here.

The challenge is therefore to develop a potent inhibitor of the HER2/HER3 heterodimer whose selectivity is independent of binding to the inactive state. In order to discover such an inhibitor we turned to a cell-based screen, which have shown a unique ability to identify novel kinase inhibitors that target the relevant conformation of a protein in its endogenous environment. A screen of approximately one million small molecules revealed a novel inhibitor whose potency and selectivity were improved through iterative rounds of medicinal chemistry. The resulting EGFR family inhibitor demonstrates the striking ability to inhibit the mutationally-activated form of HER2 as well as NRG-stabilized HER2/HER3 signaling complexes, both of which are insensitive to the clinical inhibitor lapatinib.

One unique trait of our inhibitors is their ability to interact with not only HER2, but with the kinase domain of HER3 as well.

While our approach sought to find a single agent that could address the challenge of inhibiting the active HER2/HER3 heterodimer alternative strategies using the HER2 targeting antibody pertuzumab in conjunction with T-DM1 have also been shown to be efficacious. This treatment regimen would require sufficient doses of both drugs to be consistently present for activity as either agent by itself is unable to inhibit signaling or growth of HER2 driven cells in the presence of NRG[20]. Additionally, this dual anti-body based strategy would be unable to target the p95 fragment of HER2, which is associated with trastuzumab resistance [42, 43] and poorer clinical outcomes [44, 45].

Another potential strategy to target the NRG stimulated HER2/HER3 heterodimers is to use irreversible inhibitors of HER2, for example neratinib.

F. Materials and Methods

Cell Culture and Reagents. BT-474, MCF-7, NCI-H1781, CHL-1, FaDu, were purchased from ATCC, CW-2 cells were purchased from Riken Cell Bank, HEK293T cells were purchased from the UCSF cell culture facility, and EcoPack-293 cells were purchased from Clontech. SK-BR-3 cells and parental Ba/F3 cells were obtained from UCSF. All cell lines were maintained at 37° C. and 5% CO₂. BT-474, NCI-H1781, CW-2, Axl+ Ba/F3, and 2YF/3EG Ba/F3 cells were maintained in RPMI-1640 (Gibco)+10% FBS. MCF-7, CHL-1, FaDu, HEK293T, and EcoPack-293 cells were maintained in DMEM (Gibco)+10% FBS. SK-BR-3 cells were maintained in McCoy's 5A (Gibco)+10% FBS. Parental Ba/F3's were maintained in RPMI-1640+10% FBS supplemented with 10 ng/mL IL-3. 2YF/3wt, 2YF/3TM, 2YFTM/3wt, 2YFTM/3TM, 2YF-L755S/3wt, 2YF-YVMA/3wt, and 2YF-VC/3wt Ba/F3 cells were maintained in RPMI-1640+10% FBS supplemented with 6.25 ng/mL NRG.

Lapatinib and TAK-285 were purchased from Selleckchem and were aliquoted and stored as 10 mM DMSO stocks at −20° C. Anti-phospho-EGFR (Y1068) (cat #3777), anti-EGFR (cat #4267), anti-phospho-HER2 (Y1221/Y1222) (cat #2243), anti-HER2 (cat #2165), anti-phospho-HER3(Y1289) (cat #2842), anti-HER3 (cat #12708), anti-HER4 (cat #4795), anti-phospho-Akt(T308) (cat #2965), anti-Akt (cat #2920), anti-phospho-ERK (cat #9101), anti-ERK (cat #4695), anti-phospho-S6 (S240/244) (cat #2215), anti-S6 (cat #2217), anti-phospho-4-EBP1 (T37/46) (cat #2855), anti-4EBP1 (cat #9644), anti-α-tubulin (cat #3873), and human neuregulin-1 (cat #5218) were purchased from cell signaling technologies. Mouse IL-3 (cat #PMC0034) was purchased from Gibco.

Cloning and Ba/F3 Cell Selection. Site-directed mutagenesis was performed according to standard protocols on the human HER2 and HER3 sequences in pcDNA3.1. The desired constructs were Gibson cloned into the pMSCV plasmid (Clontech) containing the puro resistance (HER3, Axl) or neo resistance (HER2) gene[46]. The sequences of all constructs were confirmed with DNA sequencing. To produce virus, EcoPack-293 cells in a 6-well plate were transfected with the desired pMSCV plasmid using lipofectamine LTX (Invitrogen) according to the manufacturer's protocol. Media was exchanged 8 h after transfection. 48 h after transfection the viral supernatant was filtered through a 0.2 μM filter and added to one well of a 6 well plate containing 2×10⁶ Ba/F3 cells in 1 mL of RPMI media containing 40% FBS, 10 ng of IL-3, and 4 μg of polybrene (Sigma). The cells were then centrifuged at 2,000×g for 90 min at room temperature, placed back in the incubator for 24 h, and then added to a T-75 flask containing fresh RPMI-1640 media supplemented with 10 ng/mL of IL-3 and incubated for an additional 24 h.

For the 2YF/3wt cells, the parental cells were first transduced with HER3 according to the protocol above and were then spun down at 500×g for 5 min and re-suspended in media supplemented with 10 ng/mL IL-3 and 3 μg/mL puromycin (Invitrogen). Cells were maintained in these conditions for seven days, passaging as required. After seven days the cells were spun down, washed with fresh media, and then used for a subsequent round of transduction according to the protocol above with HER2YF virus. 48 h after the second transduction, the cells were re-suspended in RPMI-1640 containing 10 ng/mL IL-3 and 800 μg/mL G418 (Invitrogen). The cells were maintained in these conditions for seven days, passaging as required. After seven days the cells were spun down, washed with fresh media, and then suspended in media supplemented with 10 ng/mL of NRG. Cells were maintained in these conditions for seven days to select for a NRG dependent population of 2YF/3wt Ba/F3 cells that were then maintained as described above. The same protocol was used for the 2YF-L755S/3wt, 2YF-YVMA/3wt, and 2YF-VC/3wt cell lines using the indicated constructs.

For the HER2YF/3TM, HER2YFTM/3wt, and HER2YFTM/3TM Ba/F3 cell lines, an identical protocol was used with the exception that the populations were first selected for expression of the indicated HER2 construct (G418 resistance), followed by the expression of the indicated HER3 construct (puromycin resistance).

For HER2YF/3EG Ba/F3 cells were selected according to the protocol for the HER2YF/3TM Ba/F3's with the exception that no NRG was supplemented in the media during IL-3 independent selection.

For Axl+ cells, the transduced cells were spun down at 500×g for 5 min and resuspended in media supplemented with 10 ng/mL IL-3 and 3 μg/mL puromycin (Invitrogen). Cells were maintained in these conditions for 7 days, passaging as required. After 7 days the cells were spun down, washed with fresh media, and then suspended in IL-3 free media. The cells were maintained in these conditions for 2 weeks to select for an IL-3 independent population of Axl+ driven cells.

Proliferation Assays. For adherent cell lines, cells were plated onto opaque white 96 well plates (Greiner cat #655083) and allowed to adhere overnight. The following day media was changed to fresh media that contained either DMSO or the indicated concentration of drug plus NRG (50 ng/mL final concentration) as indicated. Plates were incubated at 37° C. for 72 h and proliferation was read out using Cell Titer Glo (Promega) according to the manufacturer's protocol. For Ba/F3 cell proliferation, cells, drug dilution, and any necessary growth factors (10 ng/mL IL-3 or 6.25 ng/mL NRG) were combined in a well of a 96 well plate. Plates were incubated at 37° C. for 48 h and proliferation was read out using Cell Titer Glo according to the manufacturer's protocol. For all normalized assays, proliferation was normalized to the DMSO control condition. All graphs were plotted in GraphPad Prism 6 and fit with a non-linear regression of the log(inhibitor) vs response with a variable slope where shown. All graphs are averages (+/−standard deviation) of biological triplicates each performed in technical triplicate unless otherwise noted. If only one biological replicate was performed standard deviations were derived from the technical replicates.

Immunoblotting. Cells were grown in 6 well plates and treated according to the indicated conditions at which point the media was aspirated, cells were washed with 1 mL of cold PBS, which was then aspirated and the plates were frozen at −80° C. The frozen cells were thawed on the plates in a buffer containing 50 mM Tris pH 7.5, 150 mM NaCl, 1 mM EDTA, and 1% Triton X-100 supplemented with 1× phosphatase (PhoSTOP, Roche) and 1× protease (complete-mini tablets, Roche) inhibitors. Lysates were scraped, transferred to Eppendorf tubes, and cleared by centrifugation at 20,000×g for 20 min at 4° C. The clarified lysates were transferred to chilled, clean tubes, and normalized for protein concentration by Bradford (Bio-Rad). The normalized lysates were diluted with Laemmli loading buffer, and 10 μg of total protein was run on a 4-12% gradient gel (Invitrogen), which was then transferred to 0.45 μM nitrocellulose (Bio-Rad) and analyzed using the indicated primary antibodies according to the manufacturer's recommendations. Primary antibodies were detected using IRDye secondary antibodies (Li-Cor) according to the manufacturer's recommendations and scanned on an Odyssey imager (Li-Cor). Scanned images were cropped and assembled in Adobe Illustrator 6.

For the HER3 immunoprecipitation cells were treated the same as above but lysed in a buffer containing 20 mM Tris pH=7.5, 150 mM NaCl, 1 mM EDTA, and 1% Triton X-100 supplemented with 1× phosphatase (PhoSTOP, Roche) and 1× protease (complete-mini tablets, Roche) inhibitors. 1 mg of of the total protein was immunoprecipitated with the HER3 anti-body (CST, cat #12708) at 4° C. overnight, followed by incubation with protein A beads (CST cat #8687) for 30 min at room temperature. The beads were washed 3× with lysis buffer, eluted by boiling in 3× laemmli buffer and analyzed by western blot as detailed above.

High Throughput Screening and Analysis. For compound screening, 20 nL of 1 mM compound solutions in DMSO were transferred (Echo Labcyte) into white 1,536-well plates. Subsequently, cells were seeded in 5 μL of growth medium (500 cells per well) using an automated plate filler (Kalypsys), resulting in 4 μM compound concentration. Each assay plate included neutral (DMSO only) and inhibitor (lapatinib) control wells. CellTiter-Glo Reagent (Promega, 2 μL/well) was added two days later. Luminescence signal was read after 10 minutes using an automated plate reader (ViewLux or Envision, Perkin-Elmer). The data were analyzed using the Genedata Screener software, normalized by neutral control. The percentage inhibition for each tested compound was calculated on per-plate basis, and all compounds that showed over 50% inhibition of the luminescence signal as compared to the DMSO control were picked as primary hits for triplicate confirmation. Hits confirmed with >50% inhibition in two out of the three replicates were subsequently assayed in parental Ba/F3+IL3 cells and non-toxic hits (<30% inhibition in parental cells) were further assayed in dose response in 2YF/3wt+NRG, parental BaF3+IL-3, and BaF3-Axl cells in order to identify hits that selectively inhibit the 2YF/3wt cells in the presence of NRG.

In vitro Kinome Screen. In vitro profiling of compound 2 at 100 nM was conducted by Nanosyn.

Real Time Cell Growth Assay. CHL-1 cells were plated in clear bottom black 96 well plates (Corning cat #3904) and allowed to adhere overnight. The following day media was changed to fresh media that contained either DMSO or the indicated concentration of drug. Confluence was measured every 2 h for 96 h using 2 bright field images per well taken on an IncuCyte Zoom (Essen BioScience). Data were graphed in GraphPad Prism 6 and are averages of biological duplicates, each performed in technical triplicate.

In vitro HER2 Kinase Assay. In vitro kinase assays with the HER2 kinase domain (SignalChem) were performed in triplicate as follows. To 9 μL of a 2.5× solution of kinase and substrate in reaction buffer was added 3 μL of a 5× DMSO or inhibitor dilution in 10% DMSO:water. The inhibitor/kinase solution was incubated at room temperature for 10 minutes. The kinase assay was initiated by the addition of 3 μL of a 5× solution of ATP, and ran for 15 minutes. The final reaction conditions were 50 mM Tris (pH7.4), 5 mM MnCl₂, 0.01% Tween-20, 2 mM DTT, 100 μM E₄Y substrate (SignalChem), 15 nM HER2, 2% DMSO, 50 μM ATP, and 1 μCi γ³²P-ATP. After 15 minutes, 3 μL of each reaction was pipetted onto phosphocellulose sheets (P81, Whatman) and allowed to dry. The sheets were then washed 4×5 min with a solution of 0.5% phosphoric acid, dried, and exposed to a phosphor screen overnight. Phosphorimaging was conducted on a Typhoon 9500, image intensities were quantified in ImageQuant 5.2, normalized to the DMSO control and plotted in GraphPad Prism 6.

HER3 Thermofluor Assay. The HER3 gatekeeper mutation (T787M) was introduced into the HER3 tyrosine kinase domain in the pFastBac plasmid using standard protocols. Both wt and T787M HER3 were purified according to the previously published protocols[35]. Thermofluor reactions were performed in duplicate and set up as follows. 1 μL of an inhibitor or DMSO dilution in 40% DMSO:water was added to 19 μL of the HER3 kinase domain in reaction buffer. The final reaction solution contained 100 mM MOPS, 200 mM NaCl, 5% glycerol, 5 mM MgCl2, 0.1 mM DTT, 5× SYPRO orange, 2 μM kinase, 2% DMSO and 20 μM inhibitor in the wells of a 96-well, low profile, white, PCR plate (USA scientific). The solution was pipetted up and down to mix, sealed with TempAssure clear PCR flat caps (USA Scientific), centrifuged at 500×g for 30 s, and heated in a Stratagene Mx3005P RT-PCR machine from 25° C. to 95° C. in 0.5° C. increments every 30 s after an initial incubation at 25° C. for 10 min. Fluorescence was measured at the end of each 30 s period with an excitation wavelength of 492 nm and an emission wavelength of 610 nm. To obtain the melting temperature, fluorescent signals were normalized to the maximum fluorescent signal for that well. Values after the well had reached a maximum signal were discarded and the signals were fit to the Boltzmann equation in Graphpad Prism 6. ΔTₘ was calculated as the difference in melting temperature between the compound treated kinase compared to the DMSO control.

Transfected HER2 kinase Activity. The HER2 gatekeeper mutation (T798M) was introduced into the HER2 gene in pcDNA3.1 using standard protocols. HEK293T cells were transfected with the indicated pcDNA3.1 constructs of HER2 using Lipofectamine LTX according to the manufacturers protocol. 24 h after transfection the media was exchanged for fresh drug containing media. After 1 h of drug treatment the cells were processed for immunoblots according to the above protocol.

Cell Death Assay. Cells were plated in clear bottom, black, 96 well plates (Corning cat #3904) and allowed to adhere overnight. The following day media was changed to fresh media that contained 1× concentration of CellTox green (Promega) and either DMSO or the indicated concentration of drug and NRG. Cells were allowed to grow for 72 h after which the number of dead cells was measured using the IncuCyte Zoom. Immediately after the 72 h read, 5 of 1.25% Triton X-100 was added to each well, which were then incubated at 37° C. for 30 min to lyse all cells that were then counted by the IncuCyte. The percent cell death was calculated by dividing the number of dead cells counted at 72 h by the number of total DNA containing cells after the detergent treatment. Values are the average of biological triplicate each performed in technical triplicate and were plotted in GraphPad Prism 6.

Crystallization and Structure Determination. EGFR⁶⁷²⁻⁹⁹⁸/V924R protein expression and purification was performed exactly as previously described[34]. For the EGFR TKD^{V924R}/55A structure, EGFR TKD protein was concentrated to 8 mg/ml in 20 mM Tris-HCl, pH 8.0, containing 150 mM NaCl and 2 mM DTT. Protein was co-crystallized with excess of a drug 2 (1:2 molar ratio) in a reservoir solution of 1.34M ammonium sulfate, 1.34% (v/v) PEG 400, and 0.1 M sodium acetate/acetic acid pH 5.5 in the hanging drop at 21° C. Crystals were cryo-protected in reservoir solution with added 20% (w/v) glycerol and flash frozen in liquid nitrogen. Diffraction data were collected at beamline 23ID-B of GM/CA@APS (Advanced Photon Source), where crystals diffracted to 3.3 Å, and were processed using HKL2000 (see Table 2). The structure was solved by molecular replacement using Phaser with the active EGFR (WT) TKD structure (PDB 1M17) as an initial search model. Repeated cycles of manual building/rebuilding using Coot were alternated with rounds of refinement employing REFMAC and PHENIX, plus composite omit maps calculated using PHENIX. Coordinates, parameter files and molecular topology of compound 2 were generated by PRODRG[47]. Data collection and refinement statistics are shown in Table 2, and structural figures were generated with PyMOL.

HER3 Autophosphorylation Assay. ErbB3-ICD⁶⁶⁵⁻¹³²³ wild-type expression and purification was performed exactly as previously described[35]. To monitor the change in autophosphorylation, 1 μM ErbB3-ICD⁶⁶⁵⁻¹³²³ protein was incubated with inhibitors (varied concentrations noted in the figure) and DOGS-Ni-NTA (prepared as described in Zhang et al.[4]) in 100 mM MOPS pH 7.4, containing 200 mM NaCl, divalent cations (2 mM MnCl₂ plus 5 mM MgCl₂), 5% glycerol, 0.1 mM DTT and 200 μM ATP for 1 hour at 25 ° C., and the reactions were stopped by adding 50 mM EDTA and SDS-PAGE gel-loading buffer for rapid qualitative comparison of autophosphorylation by SDS-PAGE and immunoblotting with anti-phosphotyrosine (pY20) and anti-(His)₅.

Pharmacokinetic Evaluation of Compound 3. Pharmacokinetic profiling of compound 3 was performed by Biotranex.

G. Chemical Synthesis

General Methods: Reactions were performed in sealed vials with magnetic stirring. All commercial reagents were used without further purification. All microwave reactions were performed on a discover system (CEM). Silica gel chromatography was performed on a Combiflash Rf+using column cartridges pre-packed with 40-60 micron silica (Teledyne Isco). All RP-HPLC were performed with a Waters 2545 binary gradient module equipped with an) (Bridge prep C18 column using H2O+0.1% formic acid and CH3CN+0.1% formic acid (5-95% gradient) while monitoring at 254 nm. Low resolution mass spectra (LC/ESI-MS) were recorded in positive and negative mode on a Waters TQ detector with an Acquity UPLC equipped with a BEH C18 column. ¹H and ¹³C NMR spectra were recorded on a Bruker Avance III HD 400 spectrometer or a Bruker Avance DRX500 spectrometer and referenced to solvent peaks. Coupling constants (J) are reported in hertz, chemical shifts are reported in δ (ppm) as either s (singlet), d (doublet), t (triplet), dd (doublet of doublets), dt (doublet of triplets), or m (multiplet).

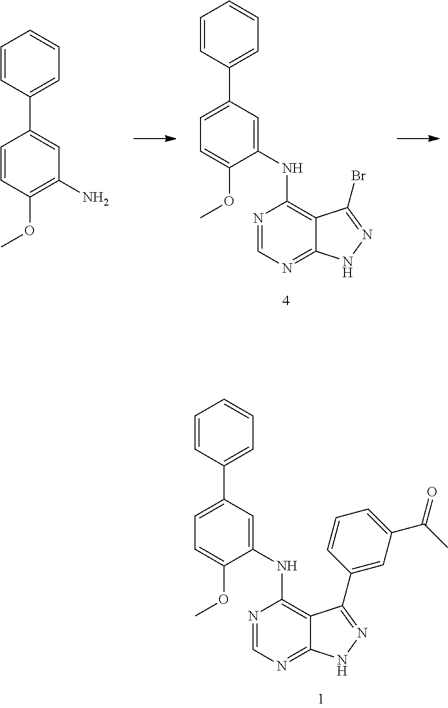 [SMILES: COc1ccc(-c2ccccc2)cc1N ; COc1ccc(-c2ccccc2)cc1Nc1ncnc2[nH]nc(Br)c12 ; COc1ccc(-c2ccccc2)cc1Nc1ncnc2[nH]nc(-c3cccc(C(C)=O)c3)c12]

1-(3-(4-((4-methoxy-[1,1′-biphenyl]-3-yl)amino)-1H-pyrazolo[3,4-d]pyrimidin-3-yl)phenyl)ethanone (1): A microwave tube containing 19.3 mg of 5-phenyl-o-anisidine (0.097 mmol), 25.1 mg of 3-bromo-4-chloro-1H-pyrazolo[3,4-d]pyrimidine (0.11 mmol), 9.2 μL of glacial acetic acid (0.16 mmol) and 1 mL of n-butanol with a stir bar was capped and heated to 130° C. for 15 min in a microwave reactor. The reaction was cooled and concentrated by rotary evaporation. The residue was recrystallized in ethanol to give 30.7 mg of crude 4 as a white solid. The crude 4 was added to a microwave tube containing 54.2 mg of 3-acetylphenyl-boronic acid (0.31 mmol), and 68.5 mg of potassium phosphate (0.31 mmol) in 1.5 mL of dioxane:H₂O (3:1) with a stir bar, which was sparged with argon for 5 min. 6.3 mg of XPhos Pd G2 (0.0078 mmol) and 7.8 mg of SPhos (0.016 mmol) were then added to the microwave tube which was purged with argon and heated to 150° C. for 20 min in a microwave. The reaction was cooled, concentrated in vacuo, and the residue was purified by silica gel chromatography (eluent, Hex:EtOAc=3:1 to 1:3) to give 26.4 mg (62.5% over 2 steps) of 1 as a white powder. MS (ES+) m/z 436.9 (M+H)⁺¹; ¹H NMR (DMSO-d₆, 400 MHz) δ 1.66 (s, 3H), 3.60 (s, 3H), 7.08 (d, 1H, J=8.6 Hz), 7.33 (m, 2H), 7.47 (t, 2H, J=7.7 Hz), 7.62 (m, 2H), 7,84 (t, 1H, J=7.7 Hz), 7.98 (s, 1H), 8.06 (dt, 1H, J=7.7 , 1.3 Hz), 8.21 (dt, 1H, J=7.8, 1.3 Hz), 8.33 (t, 1H, J=1.5 Hz), 8.60 (s, 1H), 9.12 (d, 1H, J=2.2 Hz), 14.04 (s, 1H); ¹³C NMR (DMSO-d6, 400 MHz) δ 27.29, 56.19, 99.25, 111.45, 118.68, 121.87, 126.84, 127.38, 128.70, 128.86, 129.08, 129.40, 130.35, 133.3, 133.73, 133.97, 137.98, 140.71, 143.5, 148.09, 154.45, 155.99, 156.1, 198.1.

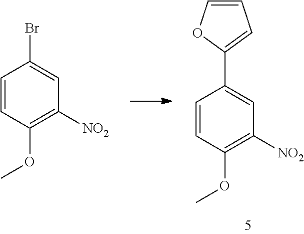 [SMILES: COc1ccc(Br)cc1[N+](=O)[O-] ; COc1ccc(-c2ccco2)cc1[N+](=O)[O-]]

2-(4-methoxy-3-nitrophenyl)furan (5): A vial containing a stir bar and a solution of 166.3 mg of 4-bromo-2-nitroanisole (0.72 mmol) and 247.6 mg 2-Furanylboronic acid MIDA ester (1.11 mmol) in 4.35 mL of 1,2 dimethoxyethane and 2.9 mL of 1M Na₂CO₃ in H₂O was sparged with argon for 5 min. 172 mg of Tetrakis(triphenylphosphine)palladium(0) (0.15 mmol) was added and the reaction, which was purged with argon and stirred at 80° C. for 12 h. The reaction was cooled and water was added to the reaction mixture, which was extracted 3 times with dichloromethane. The combined organic layer was washed with brine, dried with anhydrous sodium sulfate, filtered and concentrated by rotary evaporation. The residue was purified by silica gel chromatography (eluent, Hex:EtOAc=1:0 to 3:1) to give 140.4 mg (89%) of 5 as a yellow solid. MS (ES+) m/z 220.4 (M+H)⁺¹; ¹H NMR (DMSO-d₆, 400 MHz) δ 3.96, (s, 3H), 6.62 (dd, 1H, J=3.4, 1.8 Hz). 7.03 (dd, 1H, J=3.4, 0.7 Hz), 7.44 (d, 1H, J=9.1 Hz), 7.77 (dd, 1H, J=1.8, 0.7 Hz), 7.97 (dd, 1H, J=8.8, 2.3 Hz), 8.17 (d, 1H, J=2.3 Hz); ¹³C NMR(DMSO-d6, 500 MHz) δ 57.31, 106.85, 112.74, 115.50, 120.06, 123.67, 129.43, 140.11, 143.72, 151.28, 151.37.

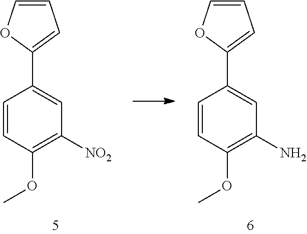 [SMILES: COc1ccc(-c2ccco2)cc1[N+](=O)[O-] ; COc1ccc(-c2ccco2)cc1N]

5-(furan-2-yl)-2-methoxyaniline (6): To a vial containing a stir bar and 131.4 mg of 5 (0.6 mmol) in 2 mL THF was added 686 μL glacial acetic acid (12 mmol). The solution was stirred at 0° C. for 5 min and 1.178 g of powdered zinc (18 mmol) was added. The reaction was allowed to warm to room temperature over 1 h. The reaction was then diluted with MeOH, filtered, and concentrated in vacuo. The resulting residue was extracted from saturated sodium bicarbonate with 3 portions of dichloromethane. The pooled organic layer was dried with anhydrous sodium sulfate, filtered, and concentrated to give 94.4 mg (83%) of 6 as an off white solid. MS (ES+) m/z 190.6 (M+H)⁺¹; ¹H NMR (DMSO-d₆, 400 MHz) δ 3.79 (s, 3H), 4.82 (s, 2H), 6.51 (dd, 1H, J=3.3, 1.8 Hz), 6.6 (dd, 1H, J=3.3, 0.7 Hz), 6.83 (d, 1H, J=8.5 Hz), 6.9 (dd, 1H, J=8.3, 2.1 Hz), 6.99 (d, 1H, J=2.07 Hz), 7.63 (dd, 1H, 1.7, 0.7 Hz); ¹³C NMR(DMSO-d6, 400 MHz) δ 55.82, 103.8, 109.44, 111.17, 112.22, 112.42, 123.95, 138.32, 142.08, 146.58, 154.45.

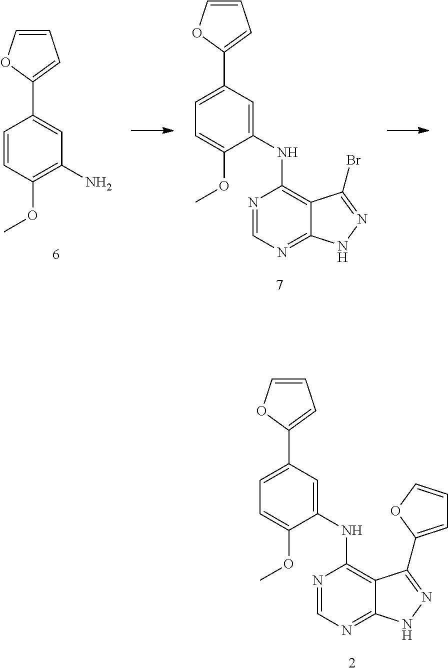 [SMILES: COc1ccc(-c2ccco2)cc1N ; COc1ccc(-c2ccco2)cc1Nc1ncnc2[nH]nc(Br)c12 ; COc1ccc(-c2ccco2)cc1Nc1ncnc2[nH]nc(-c3ccco3)c12]

3-(furan-2-yl)-N-(5-(furan-2-yl)-2-methoxyphenyl)-1H-pyrazolo[3,4-d]pyrimidin-4-amine (2): 2 was obtained in 66% yield over 2 steps as a white powder by a method similar to the one described for compound 1. MS (ES+) m/z 374.2 (M+H)⁺¹; ¹H NMR (DMSO-d₆, 400 MHz) δ 4.02 (s, 3H), 6.60 (dd, 1H, J=3.3, 1.8 Hz), 6.81 (dd, 1H, J=3.3, 0.7 Hz), 6.83 (dd, 1H, J=3.4, 1.8 Hz), 7.09 (dd, 1H, J=3.5, 0.7 Hz), 7.21 (d, 1H, J=8.7 Hz), 7.45 (dd, 1H, J=8.5, 2.1 Hz), 7.75 (dd, 1H, J=1.7, 0.6 Hz), 8.02 (dd, 1H, J=1.8, 0.7 Hz), 8.57 (s, 1H), 9.25 (d, 1H, J=2.2 Hz), 9.64 (s, 1H), 14.05 (s, 1H); ¹³C NMR(DMSO-d6, 400 MHz) δ 56.97, 97.96, 104.93, 108.49, 111.7, 112.45, 113.15, 116.44, 119.34, 123.71, 128.84, 142.76, 143.88, 147.91, 148.54, 153.77, 154.03, 155.92, 156.15.

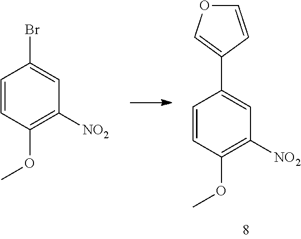 [SMILES: COc1ccc(Br)cc1[N+](=O)[O-] ; COc1ccc(-c2ccoc2)cc1[N+](=O)[O-]]

3-(4-methoxy-3-nitrophenyl)furan (8): 8 was obtained in 90% yield as a yellow solid by a method similar to the one described for compound 5. MS (ES+) m/z 220.4 (M+H)⁺¹; ¹H NMR (DMSO-d₆, 400 MHz) δ 3.95 (s, 3H), 7.04 (dd, 1H, J=1.9,0.9), 7.39 (d, 1H, J=8.8), 7.76 (t, 1H, J=1.7), 7.91 (dd, 1H, J=8.7, 2.4), 8.12 (d, 1H, J=2.4 Hz), 8.27 (m, 1H); ¹³C NMR(DMSO-d6, 500 MHz) δ 57.23, 109.05, 115.23, 121.8, 124.26, 125.26, 131.50, 140.17, 145.00, 150.94.

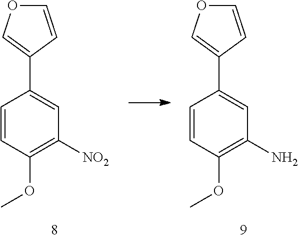 [SMILES: COc1ccc(-c2ccoc2)cc1[N+](=O)[O-] ; COc1ccc(-c2ccoc2)cc1N]

5-(furan-3-yl)-2-methoxyaniline (9): 9 was obtained in 92% yield as a pale yellow solid by a method similar to the one described for compound 6. MS (ES+) m/z 190.7 (M+H)⁺¹; ¹H NMR (DMSO-d₆, 400 MHz) δ 3.77 (s, 3H), 4.71 (s, 2H), 6.75 (m, 1H), 6.77 (d, 1H, 2.10), 6.80 (d, 1H, 8.35 Hz), 6.85 (d, 1H, J=2.0 Hz), 7.67 (t, 1H, J=1.7 Hz), 7.90 (m, 1H); ¹³C NMR(DMSO-d6, 400 MHz) δ 58.82, 109.26, 111.27, 111.60, 114.23, 125.06, 126.74, 138.20, 138.24, 144.28, 146.18.

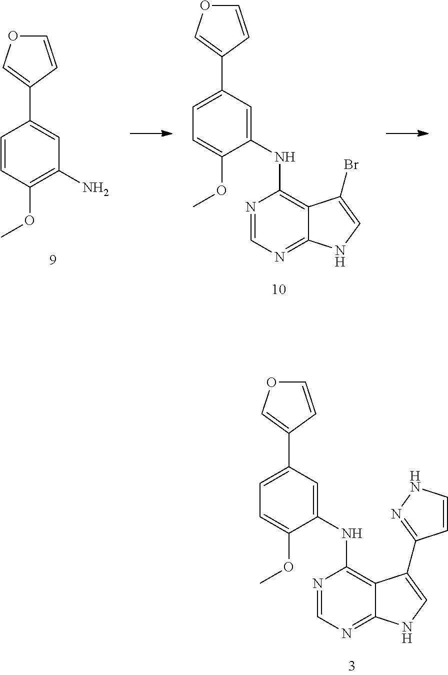 [SMILES: COc1ccc(-c2ccoc2)cc1N ; COc1ccc(-c2ccoc2)cc1Nc1ncnc2[nH]cc(Br)c12 ; COc1ccc(-c2ccoc2)cc1Nc1ncnc2[nH]cc(-c3cc[nH]n3)c12]

N-(5-(furan-3-yl)-2-methoxyphenyl)-5-(1H-pyrazol-3-yl)-7H-pyrrolo[2,3-d]pyrimidin-4-amine (3): A microwave vial containing 45.0 mg of 9 (0.24 mmol), 62.7 mg of 7-Bromo-6-chloro-7-deazapurine (0.27 mmol), 22.2 μL of glacial acetic acid (0.39 mmol) and 2.5 mL of n-butanol with a stir bar was capped and heated to 130° C. for 17 min in a microwave reactor. The reaction was cooled and concentrated by rotary evaporation. The residue was recrystallized in ethanol to give 35.5 mg of crude 10 as a brown solid. The crude 10 was added to a microwave tube containing 70.2 mg of 1H-Pyrazole-5-boronic acid pinacol ester (0.36 mmol), and 81 mg of potassium phosphate (0.38 mmol) in 1 mL of dioxane:H₂O (3:1) with a stir bar, which was sparged with argon for 5 min. 7.5 mg of XPhos Pd G2 (0.0097 mmol) and 7.3 mg of SPhos (0.018 mmol) were then added to the microwave tube which was purged with argon and heated to 150° C. for 20 min in a microwave. The reaction was cooled and run on RP-HPLC. The product containing fractions were concentrated in vacuo, and the residue was purified by silica gel chromatography (eluent, Hex:EtOAc=3:1 to 0:1) to give 12.4 mg (15% over 2 steps) of 3 as an off white powder. MS (ES−) m/z 371.6 (M−H)⁻¹; ¹H NMR (DMSO-d₆, 400 MHz) δ 3.92 (s, 3H), 6.72 (d, 1H, J=2.2), 6.87 (dd, 1H, J=1.8, 0.8 Hz), 7.08 (d, 1H, J=8.60 Hz), 7.27 (dd, 1H, J=8.3, 2.2 Hz), 7.73 (m, 2H), 7.83 (d, 1H, J=2.3 Hz), 8.04 (m, 1H), 8.28 (s, 1H), 8.78 (d, 1H, J=2.2 Hz), 11.67 (s, 1H), 11.93 (s, 1H), 12.79 (s, 1H);); ¹³C NMR (DMSO-d6, 400 MHz) 656.63, 101.33, 101.94, 109.20, 109.40, 111.79, 120.34, 120.62, 121.11, 124.46, 126.60, 130.01, 130.65, 138.67, 144.51, 146.87, 149.99, 151.86, 152.14, 154.94.

Irreversible inhibitor design and synthesis for compound 8156:

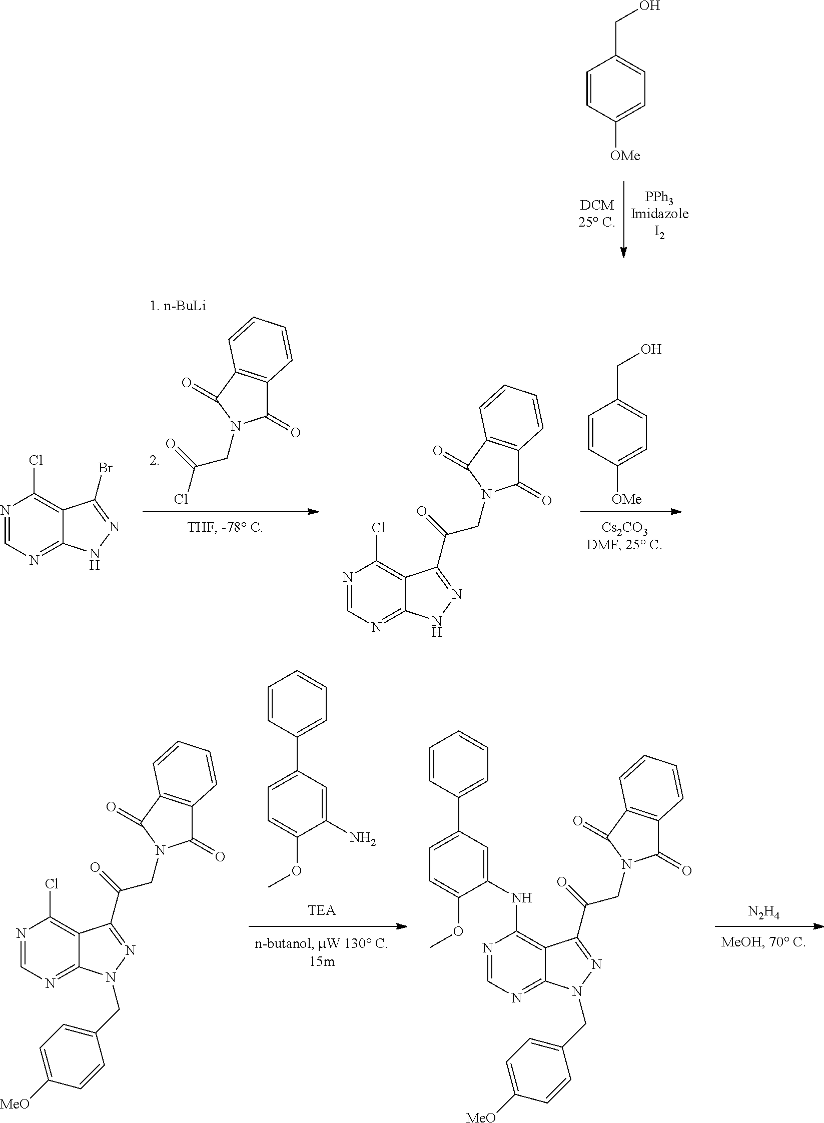

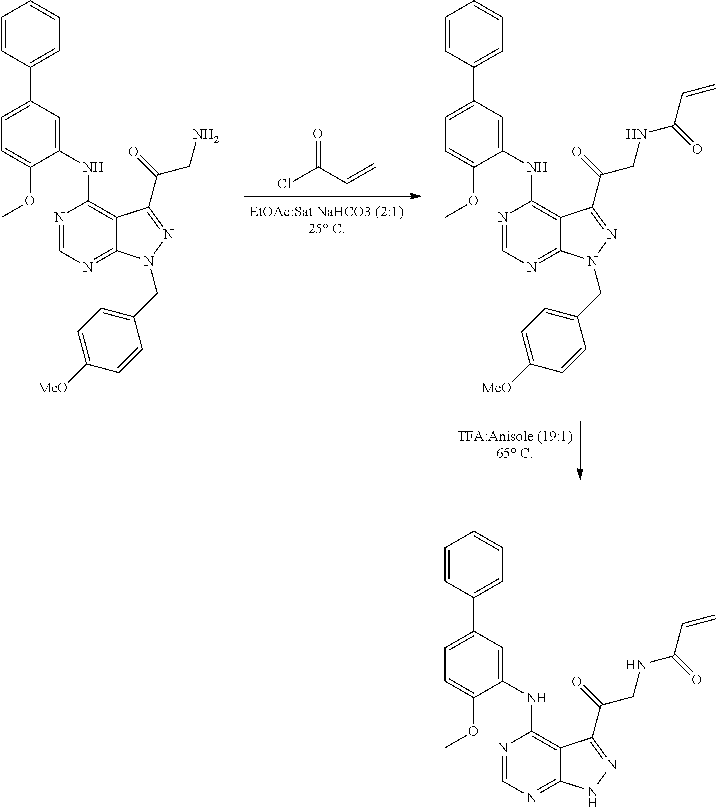

H. Inhibition Data

TABLE 7
Assay results.

| | Ba/F3 line IC50 (nm) | | | |
|---|---|---|---|---|
| | Parental + | HER2YF/HER3 + | HER2+/ | |
| Cmpd | IL-3 | NRG | 2VE | Axl+ |
| 184 | 9950 | 386 | 1290 | 527 |
| 185 | 1487 | 148 | 243 | 257 |
| 186A | >10,000 | >10,000 | >10,000 | >10,000 |
| 186B | >10,000 | >10,000 | >10,000 | >10,000 |
| 187A | >10,000 | >10,000 | >10,000 | >10,000 |
| 187B | >10,000 | >10,000 | >10,000 | >10,000 |
| 188A | >10,000 | >10,000 | >10,000 | >10,000 |
| 188B | >10,000 | >10,000 | >10,000 | >10,000 |
| 189A | 1120 | 361 | 613 | 362 |
| 189B | >10,000 | >10,000 | >10,000 | >10,000 |
| 190A | 450 | 86.8 | 229 | 171 |
| 190C | 1510 | 862 | 1840 | 643 |
| 190D | >10,000 | >10,000 | >10,000 | >10,000 |
| 191A | 2910 | 150 | 769 | 506 |
| 191B | >1,000 | >1,000 | >1,000 | >1,000 |
| 191D | 1740 | 884 | 1111 | 1010 |
| 191E | >10,000 | >10,000 | >10,000 | >10,000 |
| 191F | 2510 | 376 | 1700 | 2150 |
| 191H | >10,000 | >10,000 | >10,000 | >10,000 |
| 5-001A | >10,000 | >3,333 | >3,333 | >3,333 |
| 5-001B | >10,000 | >10,000 | >10,000 | >10,000 |
| 5-004 | >3,333 | 2590 | 3150 | 6940 |
| 39A | >10,000 | >3,333 | >10,000 | >10,000 |
| 39B | >3,333 | >3,333 | >3,333 | 1757 |
| 39C | >10,000 | >10,000 | >10,000 | >10,000 |
| 39D | >10,000 | >10,000 | >10,000 | >10,000 |
| 6 | >10,000 | >10,000 | >10,000 | >10,000 |
| 41A | >10,000 | >3,333 | >10,000 | >10,000 |
| 41B | >3,333 | >3,333 | >1,000 | >1,000 |
| 42 | >10,000 | >10,000 | >10,000 | >10,000 |
| 43 | >3,333 | >3,333 | >3,333 | >3,333 |
| 13 | >10,000 | 839 | 3396 | 2957 |
| 45A | >10,000 | 43 | 978 | 1206 |
| 45B | >10,000 | 745 | 3607 | 4025 |
| 45C | >10,000 | 952 | >3,333 | >3,333 |
| 45D | 3014 | 270 | 869 | 880 |
| 45E | >10,000 | 53 | 1349 | >10,000 |
| 45F | >10,000 | 739 | 1022 | 535 |
| 53B | >10,000 | 13.9 | 83.5 | >10,000 |
| 55A | >10,000 | 39.5 | 908.3 | >3,333 |
| 57A | >10,000 | 21 | 459.9 | >10,000 |
| 57B | >10,000 | 12.6 | 82.9 | >10,000 |
| 65 | 8.4 | 4 | 11 | |
| 66A | | 1560 | 2336 | |
| 66B | | 31 | 210 | |
| 66C | | 362 | 642 | |
| 144A | >3,333 | 170.7 | 1083 | |
| 144B | >3,333 | 21.4 | 307.5 | |
| 152 | >10,000 | 822 | 2568 | |
| 154A | >10,000 | >10,000 | >10,000 | |
| 154B | >10,000 | 39.3 | 294.7 | |
| 154C | >10,000 | >10,000 | >10,000 | |
| 147 | 1360 | 58.7 | 359 | |
| 153 | >3,333 | 17.53333333 | 285 | |
| 170 | | 687 | 673 | |
| 171 | | 45 | 183 | |
| 172 | | 910 | 422 | |
| 173B | | 32 | 228 | |
| 176 | 2288 | 29 | 216.9 | |
| 178 | 2536.7 | 6.8 | 70.3 | 1172.3 |

TABLE 8
Assay data. Compounds in the table below may be written with a dash, e.g., 8-089, or
without a dash, e.g., 8089, and it is understood these represent identical compounds.

| | Ba/F3 line IC50 (nm) | | | | | | |
|---|---|---|---|---|---|---|---|
| | | HER2YF/ | | | HCC827 | | |
| Cmpd | Parental + IL-3 | HER3 + NRG | HER2+/2VE | Axl+ | (% viable at 1 nM) | A431 | NCI-H1975 |
| 8-089 | 2443 | 20.8 | 118 | 1155 | 99.9 ± 14.6% | 914 ± 134 | 2248 ± 54 |
| | | | | | (3) | (3) | (3) |
| 8-090 | 2252 | 227 | 895 | 1567 | 102% | 911 | 2544 |
| | | | | | (1) | (1) | (1) |
| 8-091 | 2586 | 9.25 | 120 | 696.5 | 80.9 ± 24% | 634 ± 188 | 1418 ± 117 |
| | | | | | (3) | (3) | (3) |
| 8-092 | >3333 | 20.5 | 231.5 | 1197.5 | 81.6 ± 21.1% | 762 ± 215 | 1900 ± 134 |
| | | | | | (3) | (3) | (3) |
| 8-095 | >3333 | 5 | 46.7 | 1314.5 | 49.2 ± 13.7% | 1116 ± 59.0 | 1740 ± 157 |
| | | | | | (3) | (3) | (3) |
| 8-096 | 1538.5 | 2 | 31.4 | 501 | 30.6 ± 4.2% | 471 ± 63.4 | 891 ± 18.6 |
| | | | | | (3) | (3) | (3) |
| 8-097 | 1769 | 1 | 13.7 | 611.5 | 29.5 ± 7.1% | 290 ± 71 | 507 ± 85.1 |
| | | | | | (3) | (3) | (3) |
| 104A | 1595.5 | 1.5 | 48.5 | 843.9 | 29.8 ± 2.7% | 330 ± 125 | 720 ± 30 |
| | | | | | (3) | (3) | (3) |
| 104B | ~2000 | 1.5 | 46.4 | 1558 | 25.2 ± 3.4% | 343 ± 169 | 814 ± 59 |
| | | | | | (3) | (3) | (3) |

I. Pharmacokinetics and Stability Studies

TABLE 9A
A pharmacokinetic study for compound 8168B following IV administration in Male
CD-1 mice. Study details include: Animal Species/Strain/Sex: Mice/CD-1/Male;
Number of Animals: 3; Route of Administration: IV; Sampling Method: Retro-Orbital;
Feeding Condition: Fasted; Dose (mg/kg): 2; Dose Volume (mL/kg): 5; Concentration
(mg/mL): 0.4; Formulation Vehicle: Solution in 22% PEG300/15% DMSO/63% PBS;
Sample/Collection - Type: Blood collection; Anti-coagulant: K2EDTA; Sampling
Time points: Blood: 0.083, 0.25 h, 1 h, 3 h, 5 h, 8 h, 24 h.

| | |
|---|---|
| Test Article Name (IV) | Compound 8168B |
| Formulation IV solution | Solution in 22% PEG300/15% DMSO/63% PBS |
| Species | Male CD-1 mice |
| Study Design | Pharmacokinetic Study |
| Matrix | Plasma |

Bioanalytical Details
Analyte 8168B

| | | | |
|---|---|---|---|
| LLOQ (Plasma) | 1.05 ng/mL | ULOQ | 1044.00 ng/mL |

Plasma Concentration (ng/mL) of 8168B (IV-2.0 mg/kg)

| Time (h) | M1 | M2 | M3 | Mean | Std Dev | % CV |
|---|---|---|---|---|---|---|
| 0.08 | 55.13 | 53.28 | 64.54 | 58.91 | 7.96 | 13.52 |
| 0.25 | 31.90 | 29.14 | 34.06 | 31.60 | 3.48 | 11.01 |
| 1.00 | 12.34 | 22.71 | 12.93 | 17.82 | 6.92 | 38.81 |
| 3.00 | 6.96 | 11.36 | 8.08 | 9.72 | 2.32 | 23.86 |
| 5.00 | 7.32 | 7.57 | 6.55 | 7.06 | 0.72 | 10.22 |
| 8.00 | 6.02 | 7.32 | 9.38 | 8.35 | 1.46 | 17.44 |
| 24.00 | 6.79 | 5.33 | 3.54 | 4.44 | 1.27 | 28.54 |
| Dose (mg/kg)* | 2.00 | 2.00 | 2.00 | 2.00 | 0.00 | 0.00 |
| C0 (ng/mL) | 72.36 | 71.91 | 88.67 | 77.65 | 11.85 | 15.26 |
| T½ (h)# | 302.57 | 36.64 | 18.98 | 27.81 | 12.49 | 44.92 |
| AUC0-last (ng · h/mL) | 183.16 | 205.32 | 185.37 | 191.28 | 14.11 | 7.37 |
| AUCall (ng · h/mL) | 183.16 | 205.32 | 185.37 | 191.28 | 14.11 | 7.37 |
| AUC0-inf (ng · h/mL)# | 3147.06 | 487.06 | 282.28 | 384.67 | 144.80 | 37.64 |
| AUCExtra(%) | 94.18 | 57.85 | 34.33 | 46.09 | 16.63 | 36.08 |
| Cl (ml/min/kg)# | 10.59 | 68.44 | 118.09 | 93.26 | 35.11 | 37.64 |
| Vd (L/kg)# | 277.41 | 217.05 | 193.96 | 205.51 | 16.33 | 7.95 |
| MRT0-last (h) | 10.30 | 9.04 | 9.04 | 9.46 | 0.00 | 0.03 |
| Rsq | 0.4805 | 0.9963 | 0.9744 | 0.99 | 0.02 | 1.58 |

After dose formulation is 87% so under the inhouse formulation accuracy limits. PK Parameters were calculated by considering 100% formulation accuracy.

TABLE 9B
A pharmacokinetic study for compound 8168B following IP administration in Male
CD-1 mice. Study details include: Animal Species/Strain/Sex: Mice/CD-1/Male; Test
Item: sCCX155; Number of Animals: 3; Route of Administration: IP; Sampling Method:
Retro-Orbital; Feeding Condition: Fasted; Dose (mg/kg): 2; Dose Volume (mL/kg):
5; Concentration (mg/mL): 0.4; Formulation Vehicle: Solution in 22% PEG300/15%
DMSO/63% PBS; Sample/Collection - Type: Blood collection; Anti-coagulant: K2EDTA;
Sampling Time points: Blood: 0.25 h, 1 h, 3 h, 5 h, 8 h, 24 h.

| | |
|---|---|
| Test Article Name (IP) | Compound 8168B |
| Formulation IP solution | Solution in 22% PEG300/15% DMSO/63% PBS |
| Species | Male CD-1 mice |
| Study Design | Pharmacokinetic Study |
| Matrix | Plasma |

Bioanalytical Details
Analyte 8168B

| | | | |
|---|---|---|---|
| LLOQ (Plasma) | 1.05 ng/mL | ULOQ | 1044.00 ng/mL |

Plasma Concentration (ng/mL) of 8168B (IP-2.0 mg/kg)

| Time (h) | M1 | M2 | M3 | Mean | Std Dev | % CV |
|---|---|---|---|---|---|---|
| 0.25 | 241.92 | 189.54 | 198.56 | 210.01 | 28.00 | 13.33 |
| 1.00 | 100.58 | 69.73 | 76.68 | 82.33 | 16.18 | 19.66 |
| 3.00 | 2.25 | 4.17 | 2.33 | 2.92 | 1.09 | 37.24 |
| 5.00 | BLQ | BLQ | BLQ | BLQ | NA | NA |
| 8.00 | BLQ | BLQ | BLQ | BLQ | NA | NA |
| 24.00 | BLQ | BLQ | BLQ | BLQ | NA | NA |
| Dose (mg/kg)* | 2.00 | 2.00 | 2.00 | 2.00 | 0.00 | 0.00 |
| Cmax (ng/mL) | 241.92 | 189.54 | 198.56 | 210.01 | 28.00 | 13.33 |
| Tmax (h) | 0.25 | 0.25 | 0.25 | 0.25 | 0.00 | 0.00 |
| Oral T½ (h) | 0.40 | 0.50 | 0.42 | 0.44 | 0.05 | 11.89 |
| AUC0-last (ng · h/mL) | 202.77 | 160.10 | 163.46 | 175.44 | 23.73 | 13.52 |
| AUCall (ng · h/mL) | 202.77 | 160.10 | 163.46 | 175.44 | 23.73 | 13.52 |
| AUC0-inf (ng · h/mL) | 204.07 | 163.10 | 164.87 | 177.35 | 23.16 | 13.06 |
| AUCExtra(%) | 0.63 | 1.84 | 0.86 | 1.11 | 0.64 | 57.63 |
| MRT0-last (h) | 0.76 | 0.81 | 0.76 | 0.78 | 0.03 | 4.04 |
| Rsq | 0.9920 | 0.9999 | 0.9961 | 0.9960 | 0.00 | 0.39 |

After dose formulation is 90% so PK Parameters were calculated by considering 100% formulation accuracy.

TABLE 10A
A pharmacokinetic study for compound 8168C following IV administration in Male
CD-1 mice. Study details include: Animal Species/Strain/Sex: Mice/CD-1/Male; Test
Item: sCCX156; Number of Animals: 3; Route of Administration: IV; Sampling Method:
Retro-Orbital; Feeding Condition: Fasted; Dose (mg/kg): 2; Dose Volume (mL/kg):
5; Concentration (mg/mL): 0.4; Formulation Vehicle: Solution in 22% PEG300/15%
DMSO/63% PBS; Sample/Collection - Type: Blood collection; Anti-coagulant: K2EDTA;
Sampling Time points: Blood: 0.083, 0.25 h, 1 h, 3 h, 5 h, 8 h, 24 h.

| | |
|---|---|
| Test Article Name (IV) | Compound 8168C |
| Formulation IV solution | Solution in 22% PEG300/15% DMSO/63% PBS |
| Species | Male CD-1 mice |
| Study Design | Pharmacokinetic Study |
| Matrix | Plasma |

Bioanalytical Details
Analyte 8168C

| | | | |
|---|---|---|---|
| LLOQ (Plasma) | 1.03 ng/mL | ULOQ | 1029.60 ng/mL |

Plasma Concentration (ng/mL) of 8168C (IV-2.0 mg/kg)

| Time (h) | M1 | M2 | M3 | Mean | Std Dev | % CV |
|---|---|---|---|---|---|---|
| 0.08 | 601.37 | 643.79 | 668.53 | 637.90 | 33.97 | 5.32 |
| 0.25 | 381.91 | 361.85 | 533.91 | 425.89 | 94.08 | 22.09 |
| 1.00 | 180.89 | 124.19 | 132.77 | 145.95 | 30.56 | 20.94 |
| 3.00 | 14.30 | 16.88 | 24.59 | 18.59 | 5.35 | 28.80 |
| 5.00 | 4.53 | 8.17 | 9.69 | 7.46 | 2.65 | 35.53 |
| 8.00 | 1.79 | BLQ | 4.14 | 2.97 | 1.66 | 56.04 |
| 24.00 | BLQ | BLQ | BLQ | BLQ | NA | NA |
| Dose (mg/kg)* | 2.00 | 2.00 | 2.00 | 2.00 | 0.00 | 0.00 |
| C0 (ng/mL) | 753.60 | 857.24 | 747.58 | 786.14 | 61.65 | 7.84 |
| T½ (h) | 1.70 | 0.79 | 1.98 | 1.49 | 0.62 | 41.64 |
| AUC0-last (ng · h/mL) | 495.61 | 441.82 | 554.77 | 497.40 | 56.50 | 11.36 |
| AUCall (ng · h/mL) | 495.61 | 441.82 | 554.77 | 497.40 | 56.50 | 11.36 |
| AUC0-inf (ng · h/mL) | 500.01 | 451.15 | 566.58 | 505.91 | 57.94 | 11.45 |
| AUCExtra(%) | 0.88 | 2.07 | 2.08 | 1.68 | 0.69 | 41.21 |
| Cl (ml/min/kg) | 66.67 | 73.89 | 58.83 | 66.46 | 7.53 | 11.33 |
| Vd (L/kg) | 9.82 | 5.06 | 10.07 | 8.32 | 2.82 | 33.92 |
| MRT0-last (h) | 0.94 | 0.87 | 1.09 | 0.96 | 0.11 | 11.73 |
| Rsq | 0.9692 | 0.9371 | 0.9802 | 0.9622 | 0.02 | 2.33 |

After dose formulation is 85.4% so under the inhouse formulation accuracy limits. PK Parameters were calculated by considering 100% formulation accuracy. In house limit for formulation accuracy is ±20%.

TABLE 10B
A pharmacokinetic study for compound 8168C following IP administration
in Male CD-1 mice. Study details include: Animal Species/Strain/Sex:
Mice/CD-1/Male; Test Item: sCCX156; Number of Animals: 3; Route of
Administration: IP; Sampling Method: Retro-Orbital; Feeding Condition:
Fasted; Dose (mg/kg): 2; Dose Volume (mL/kg): 5; Concentration (mg/mL):
0.4; Formulation Vehicle: Solution in 22% PEG300/15% DMSO/63% PBS;
Sample/Collection - Type: Blood collection; Anti-coagulant: K2EDTA;
Sampling Time points: Blood: 0.25 h, 1 h, 3 h, 5 h, 8 h, 24 h.

| | |
|---|---|
| Test Article Name (IP) | 8168C |
| Formulation IP solution | Solution in 22% PEG300/15% DMSO/63% PBS |
| Species | Male CD-1 mice |
| Study Design | Pharmacokinetic Study |
| Matrix | Plasma |

Bioanalytical Details
Analyte 8168C

| | | | |
|---|---|---|---|
| LLOQ (Plasma) | 1.03 ng/mL | ULOQ | 1029.60 ng/mL |

Plasma Concentration (ng/mL) of 8168C (IP-2.0 mg/kg)

| Time (h) | M1 | M2 | M3 | Mean | Std Dev | % CV |
|---|---|---|---|---|---|---|
| 0.25 | 206.62 | 306.24 | 169.92 | 227.59 | 70.54 | 30.99 |
| 1.00 | 85.20 | 69.88 | 66.74 | 73.94 | 9.88 | 13.36 |
| 3.00 | 9.75 | 2.05 | BLQ | 5.90 | 5.44 | 92.28 |
| 5.00 | 2.52 | BLQ | BLQ | BLQ | NA | NA |
| 8.00 | BLQ | BLQ | BLQ | BLQ | NA | NA |
| 24.00 | BLQ | BLQ | BLQ | BLQ | NA | NA |
| Dose (mg/kg)* | 2.00 | 2.00 | 2.00 | 2.00 | 0.00 | 0.00 |
| Cmax (ng/mL) | 206.62 | 306.24 | 169.92 | 227.59 | 70.54 | 30.99 |
| Tmax (h) | 0.25 | 0.25 | 0.25 | 0.25 | 0.00 | 0.00 |
| Oral T½ (h) | 0.79 | 0.38 | NC | 0.59 | 0.29 | 48.85 |
| AUC0-last (ng · h/mL) | 208.92 | 196.69 | 104.05 | 169.89 | 57.35 | 33.76 |
| AUCall (ng · h/mL) | 208.92 | 196.69 | 104.05 | 169.89 | 57.35 | 33.76 |
| AUC0-inf (ng · h/mL) | 211.79 | 197.83 | NC | 204.81 | 9.87 | 4.82 |
| AUCExtra(%) | 1.35 | 0.57 | NC | 0.96 | 0.55 | 57.24 |
| MRT0-last (h) | 1.06 | 0.67 | 0.50 | 0.74 | 0.29 | 38.40 |
| Rsq | 0.9825 | 0.9994 | NC | 0.9909 | 0.01 | 1.21 |

After dose formulation is 110% so PK Parameters were calculated by considering 100% formulation accuracy. In house limit for formulation accuracy is ±20%.
NC:- Not calculated due to insufficient data point post Cmax.

TABLE 11
HER3 Mouse Liver Microsome stability assay study results.

| WuXi Microsomal stability | |
|---|---|
| (Mouse Extraction Ratio) | Compound name |
| <0.3 | 8168B |
| 0.4 | 8168C |
| 0.6 | 8179 |
| 0.7 | 8134 |
| 0.7 | 8186 |
| 0.8 | 8168A |
| 0.8 | 8177 |
| 0.8 | 8185 |
| 0.837 | 96 |
| 0.87 | 104A |
| 0.9 | 8164 |
| 0.9 | 8184 |
| 0.928 | 91 |
| 0.939 | 97 |
| 0.948 | 178 |

References: 1. Lemmon, M. A. et al. Cold Spring Harbor Perspectives in Biology 6, a020768-a020768 (2014). 2. Kovacs, E., Zorn, J. A., Huang, Y., Barros, T. & Kuriyan, J. Annu Rev Biochem 84, 739-764 (2015). 3. Doerner, A., Scheck, R. & Schepartz, A. Chem Biol 22, 776-784 (2015). 4. Zhang, X., Gureasko, J., Shen, K., Cole, P. A. & Kuriyan, J. Cell 125, 1137-1149 (2006). 5. Schneider, M. R. & Yarden, Y. Oncogene (2015). doi:10.1038/onc.2015.372; 6. Yarden, Y., Yarden, Y., Sliwkowski, M. X. & Sliwkowski, M. X. Nat Rev Mol Cell Biol 2, 127-137 (2001). 7. Brewer, M. R. et al. Proceedings of the National Academy of Sciences 110, E3595 (2013). 8. Wang, Z. et al. Nat Struct Mol Biol 18, 1388-1393 (2011). 9. Foster, S. A. et al. Cancer Cell doi:10.1016/j.ccell.2016.02.010.10. Lee-Hoeflich, S. T. et al. Cancer Res 68, 5878-5887 (2008). 11. Tzahar, E. et al. Mol. Cell. Biol. 16, 5276-5287 (1996). 12. Russo, P. et al. Cancer Res 72, 2672-2682 (2012). 13. Jaiswal, B. S. et al. Cancer Cell 23, 603-617 (2013). 14. Lee, D. Y., Wilson, T. R., Shames, D. S., Berry, L. & Settleman, J. Cancer Cell 20, 158-172 (2011). 15. Bose, R. et al. Cancer Discovery 3, 224 (2012). 16. Wang, S. E. et al. Cancer Cell 10, 25-38 (2006). 17. Greulich, H. et al. Proc Natl Acad Sci USA 109, 14476-14481 (2012). 18. Geyer, C. E. et al. N Engl J Med 355, 2733-2743 (2006). 19. Verma, S. et al. N Engl J Med 367, 1783-1791 (2012). 20. Lewis Phillips, G. D. et al. Clinical Cancer Research 20, 456 (2013). 21. Wilson, T. R. et al. Nature 487, 505-509 (2012). 22. Sergina, N. V. et al. Nature 445, 437 (2007). 23. Chakrabarty, A., Sanchez, V., Kuba, M. G., Rinehart, C. & Arteaga, C. L. Proceedings of the National Academy of Sciences 109, 2718 (2011). 24. Chandarlapaty, S. et al. Cancer Cell 19, 58-71 (2011). 25. Amin, D. N. et al. Sci Transl Med 2, 16ra7 (2010). 26. Das, P. M. et al. Oncogene 29, 5214-5219 (2010). 27. Sartor, C. I. et al. MCB 21, 4265-4275 (2001). 28. Aertgeerts, K. et al. J Biol Chem 286, 18756-18765 (2011). 29. Wood, E. R. et al. Cancer Res 64, 6652-6659 (2004). 30. Littlefield, P. et al. Sci Signal 7, ra114-ral 14 (2014). 31. Brewer, M. R. et al. Mol Cell 34, 641 (2009). 32. Warmuth, M., Kim, S., Gu, X.-J., Xia, G. & Adrian, Current Opinion in Oncology 19, 55-60 (2006). 33. Jura, N. et al. Cell 137,1293-1307 (2009). 34. Park, J. H., Liu, Y., Lemmon, M. A. & Radhakrishnan, R. Biochem J 448, 417-423 (2012). 35. Shi, F., Telesco, S. E., Liu, Y., Radhakrishnan, R. & Lemmon, M. A. Proc Natl Acad Sci USA 107, 7692-7697 (2010). 36. Jura, N. et al. Proc Natl Acad Sci USA 106, 21608-21613 (2009). 37. Rexer, B. N. et al. Clin Cancer Res 19, 5390-5401 (2013). 38. Vivanco, I. et al. Cancer Discovery 2, 458-471 (2012). 39. Yoshida, T., Kakegawa, J., Yamaguchi, T. & Hantani, Y. Oncotarget (2012). 40. Adrián, F. J. et al. Nature Chemical Biology 2, 95-102 (2006). 41. Xie, T. et al. Nature Chemical Biology 10, 1006 (2014). 42. Scaltriti, M. et al. J Natl Cancer Inst 99, 628-638 (2007). 43. Chandarlapaty, S. et al. Oncogene 29, 325-334 (2009). 44. Sperinde, J. et al. Clin Cancer Res 16, 4226-4235 (2010). 45. Sáez, R. et al. Clin Cancer Res 12, 424-431 (2006). 46. Gibson, D. G. et al. Nat Meth 6, 343-345 (2009). 47. Schüttelkopf, A. W. & van Aalten, D. M. F. Acta Crystallogr D Biol Crystallogr 60, 1355-1363 (2004).

What is claimed is:

1. A compound having the formula: 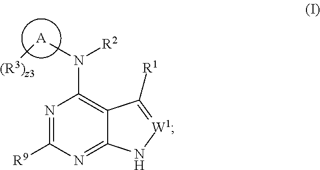
wherein
Ring A is aryl or heteroaryl;
W¹ is N or C(H);
R¹ is hydrogen, -L¹-L²-E, substituted or unsubstituted aryl, or substituted or unsubstituted heteroaryl;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂ X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2;
wherein the compound is not

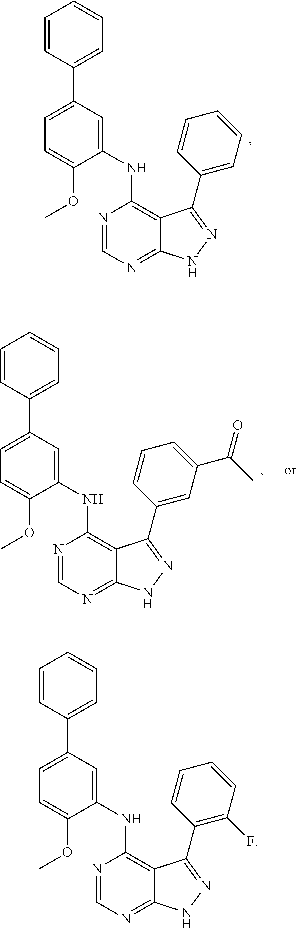 [SMILES: COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12 ; COc1ccc(-c2ccccc2)cc1Nc1ncnc2[nH]nc(-c3ccccc3F)c12]

2. A compound of claim 1, having the formula: 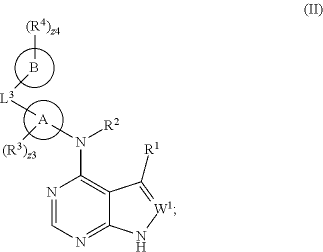
wherein
Ring B is cycloalkyl, heterocycloalkyl, aryl, or heteroaryl;
L³ is a bond, —S(O)₂—, —NR⁸—, —O—, —S—, —C(O)—, —C(O)NR⁸—, —NR⁸C(O)—, —NR⁸C(O)NH—, —NHC(O)NR⁸—, —C(O)O—, —OC(O)—, substituted or unsubstituted alkylene, or substituted or unsubstituted heteroalkyl ene;
R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}, R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z4 is an integer from 0 to 5;
Each R^{4A}, R^{4B}, R^{4C}, R^{4D}, R^{8A}, R^{8B}, R^{8C}, and R^{8D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl;
each X⁴ and X⁸ are independently —F, —Cl, —Br, or —I;
n4 and n8 are independently an integer from 0 to 4; and
m4, m8, v4, and v8, are independently an integer from 1 to 2.

3. The compound of claim 2, having the formula:

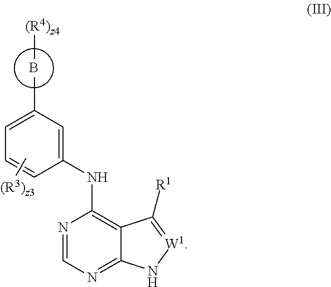

4. The compound of claim 2, having the formula:

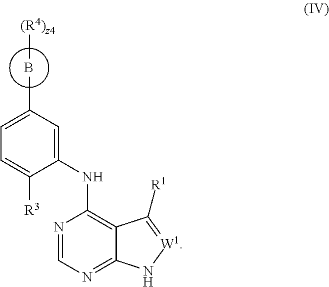

5. The compound of claim 2, wherein R⁴ is independently halogen, —CX⁴₃, —CHX⁴₂, —CH₂X⁴, —OCX⁴₃, —OCH₂X⁴, —OCHX⁴₂, or —CN.

6. The compound of claim 2, wherein R⁴ is independently halogen, —CX⁴₃, —CHX⁴₂, or —CH₂X⁴.

7. The compound of claim 2, wherein R⁴ is independently halogen.

8. The compound of claim 2, having the formula:

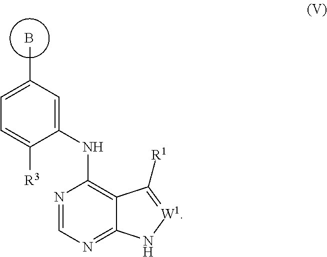

9. The compound of claim 1, wherein W¹ is C(H).

10. The compound of claim 1, wherein W¹ is N.

11. The compound of claim 1, wherein R³ is an unsubstituted heteroalkyl.

12. The compound of claim 1, wherein R³ is an unsubstituted 2 to 5 membered heteroalkyl.

13. The compound of claim 1, wherein R³ is —OCH₃, —OCH₂CH₃, —N(CH₃)₂, —NH₂, —NH(CH₃), —N(CH₂CH₃)₂, —NH(CH₂CH₃), —OCX³₃, —OCH₂X³, —OCHX³₂, or —SH.

14. The compound of claim 1, wherein R³ is —OCH₃, —OCH₂CH₃, —N(CH₃)₂, —OCX³₃, —OCH₂X³, or —OCHX³₂.

15. The compound of claim 2, wherein Ring B is substituted or unsubstituted aryl or heteroaryl.

16. The compound of claim 2, wherein Ring B is substituted or unsubstituted phenyl or substituted or unsubstituted 5 to 6 membered heteroaryl.

17. The compound of claim 2, wherein Ring B is substituted or unsubstituted phenyl.

18. The compound of claim 2, wherein Ring B is substituted or unsubstituted 5 to 6 membered heteroaryl.

19. The compound of claim 2, wherein Ring B is substituted or unsubstituted pyrazinyl, pyrimidinyl, furanyl, thienyl, pyrrolyl, imidazolyl, pyrazolyl, oxazolyl, isoxazolyl, or thiazolyl.

20. The compound of claim 1, wherein R¹ is substituted or unsubstituted aryl or substituted or unsubstituted heteroaryl.

21. The compound of claim 1, wherein R¹ is substituted or unsubstituted phenyl or substituted or unsubstituted 5 to 6 membered heteroaryl.

22. The compound of claim 1, wherein R¹ is substituted or unsubstituted phenyl.

23. The compound of claim 1, wherein R¹ is an unsubstituted phenyl.

24. The compound of claim 1, wherein R¹ is substituted or unsubstituted 5 to 6 membered heteroaryl.

25. The compound of claim 1, wherein R¹ is an unsubstituted 5 to 6 membered heteroaryl.

26. The compound of claim 1, wherein R¹ is substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted oxazolyl, substituted or unsubstituted isoxazolyl, or substituted or unsubstituted thiazolyl.

27. The compound of claim 1, wherein R¹ is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

28. The compound of claim 1, wherein R¹ is -L¹-L²-E.

29. The compound of claim 1, wherein L¹ is a bond, —C(O)—, substituted or unsubstituted alkylene, or substituted or unsubstituted heteroalkylene.

30. The compound of claim 1, wherein L¹ is a substituted or unsubstituted C₁-C₄ alkylene.

31. The compound of claim 1, wherein L¹ is —C(O)CH₂CH₂CH₂—, —C(O)CH₂CH₂—, or —C(O)CH₂—.

32. The compound of claim 1, wherein L² is —NR⁷—, substituted or unsubstituted heteroalkylene, or substituted or unsubstituted heterocycloalkylene.

33. The compound of claim 1, wherein L² is —NH—.

34. The compound of claim 1, wherein E is a covalent cysteine modifier moiety.

35. The compound of claim 1, wherein E is: 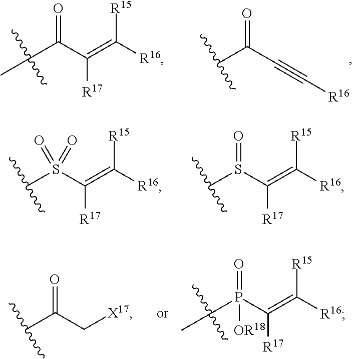
R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;
each X, X¹⁵, X¹⁶, X¹⁷ and X¹⁸ is independently —F, —Cl, —Br, or —I;
n15, n16, n17, v15, v16, and v17, are independently an integer from 0 to 4; and
m15, m16, and m17 are independently and integer from 1 to 2.

36. The compound of claim 35, wherein R¹⁵, R¹⁶, R¹⁷, and R¹⁸ are hydrogen.

37. The compound of claim 35, wherein E is:

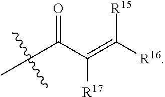

38. The compound of claim 37, wherein
R¹⁵ is hydrogen;
R¹⁶ is hydrogen, —CH₃, or —CH₂NR^{16A}R^{16B};
R¹⁷ is hydrogen; and
R^{16A} and R^{16B} are independently hydrogen or unsubstituted alkyl.

39. The compound of claim 38, wherein R^{16A} and R^{16B} are independently unsubstituted methyl.

40. The compound of claim 37, wherein
R¹⁵ is hydrogen;
R¹⁶ is hydrogen;
R¹⁷ is hydrogen, —CH₃, or —CH₂NR^{17A}R^{17B}; and
R^{17A} and R^{17B} are independently hydrogen or unsubstituted alkyl.

41. The compound of claim 40, wherein R^{17A} and R^{17B} are independently unsubstituted methyl.

42. The compound of claim 37, wherein
R¹⁵ is hydrogen, —CH₃, or —CH₂NR^{15A}R^{15B};
R¹⁶ is hydrogen;
R¹⁷ is hydrogen; and
R^{15A} and R^{15B} are independently hydrogen or unsubstituted alkyl.

43. The compound of claim 42, wherein R^{15A} and R^{15B} are independently unsubstituted methyl.

44. The compound of claim 1,wherein the compound has the formula:

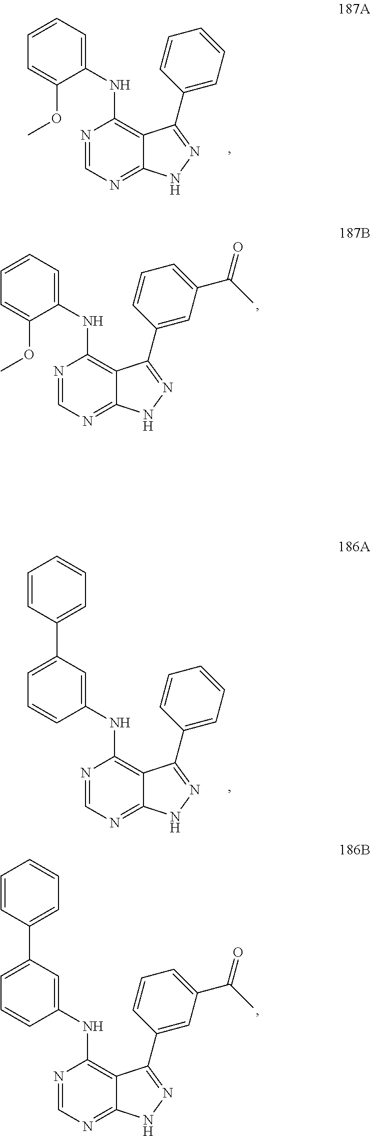 [SMILES: COc1ccccc1Nc1ncnc2[nH]nc(-c3ccccc3)c12 ; COc1ccccc1Nc1ncnc2[nH]nc(-c3cccc(C(C)=O)c3)c12 ; c1ccc(-c2cccc(Nc3ncnc4[nH]nc(-c5ccccc5)c34)c2)cc1 ; CC(=O)c1cccc(-c2n[nH]c3ncnc(Nc4cccc(-c5ccccc5)c4)c23)c1]

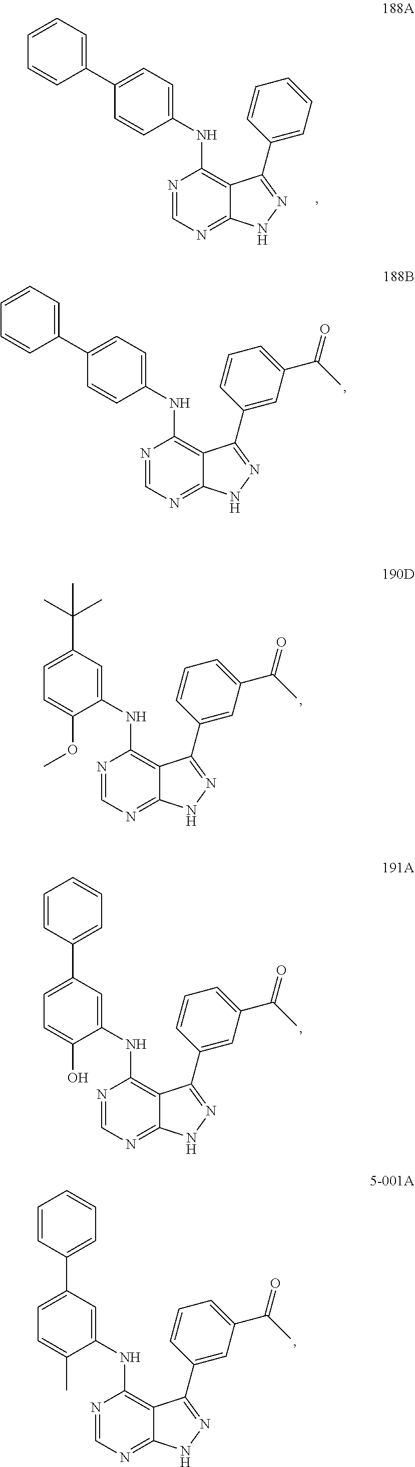 [SMILES: c1ccc(-c2ccc(Nc3ncnc4[nH]nc(-c5ccccc5)c34)cc2)cc1 ; CC(=O)c1cccc(-c2n[nH]c3ncnc(Nc4ccc(-c5ccccc5)cc4)c23)c1 ; COc1ccc(C(C)(C)C)cc1Nc1ncnc2[nH]nc(-c3cccc(C(C)=O)c3)c12 ; CC(=O)c1cccc(-c2n[nH]c3ncnc(Nc4cc(-c5ccccc5)ccc4O)c23)c1 ; CC(=O)c1cccc(-c2n[nH]c3ncnc(Nc4cc(-c5ccccc5)ccc4C)c23)c1]

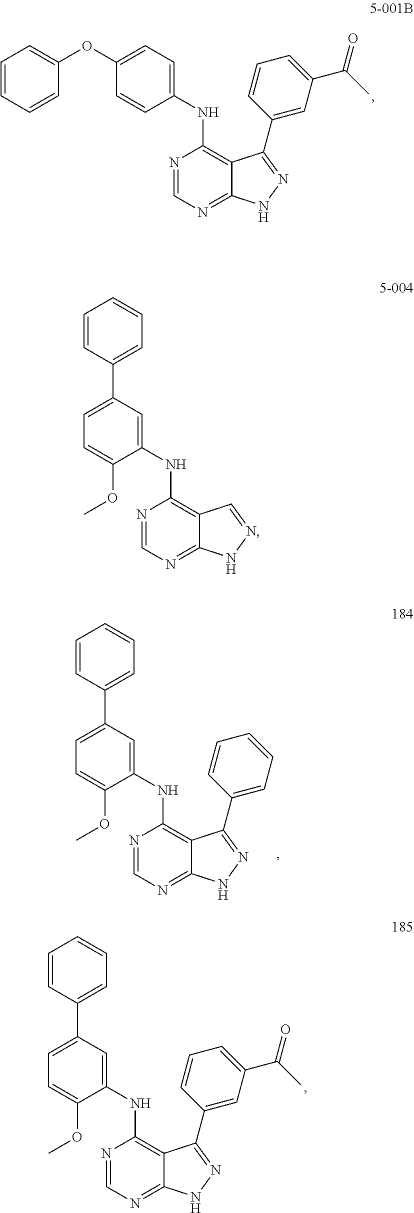 [SMILES: CC(=O)c1cccc(-c2n[nH]c3ncnc(Nc4ccc(Oc5ccccc5)cc4)c23)c1 ; COc1ccc(-c2ccccc2)cc1Nc1ncnc2[nH]ncc12 ; COc1ccc(-c2ccccc2)cc1Nc1ncnc2[nH]nc(-c3ccccc3)c12 ; COc1ccc(-c2ccccc2)cc1Nc1ncnc2[nH]nc(-c3cccc(C(C)=O)c3)c12]

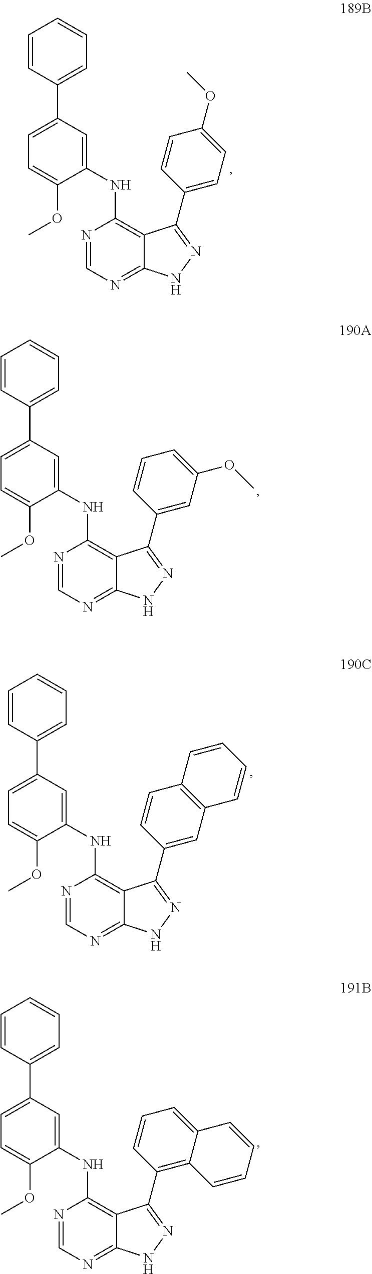 [SMILES: COc1ccc(-c2n[nH]c3ncnc(Nc4cc(-c5ccccc5)ccc4OC)c23)cc1 ; COc1cccc(-c2n[nH]c3ncnc(Nc4cc(-c5ccccc5)ccc4OC)c23)c1 ; COc1ccc(-c2ccccc2)cc1Nc1ncnc2[nH]nc(-c3ccc4ccccc4c3)c12 ; COc1ccc(-c2ccccc2)cc1Nc1ncnc2[nH]nc(-c3cccc4ccccc34)c12]

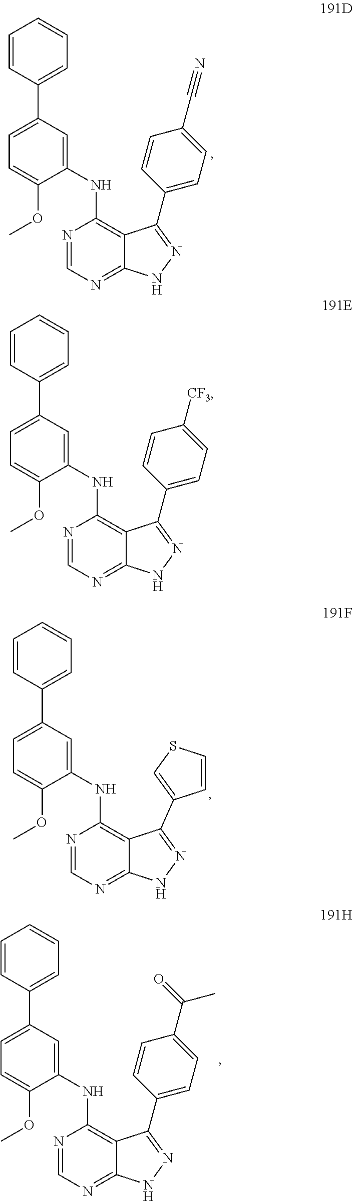 [SMILES: COc1ccc(-c2ccccc2)cc1Nc1ncnc2[nH]nc(-c3ccc(C#N)cc3)c12 ; COc1ccc(-c2ccccc2)cc1Nc1ncnc2[nH]nc(-c3ccc(C(F)(F)F)cc3)c12 ; COc1ccc(-c2ccccc2)cc1Nc1ncnc2[nH]nc(-c3ccsc3)c12 ; COc1ccc(-c2ccccc2)cc1Nc1ncnc2[nH]nc(-c3ccc(C(C)=O)cc3)c12]

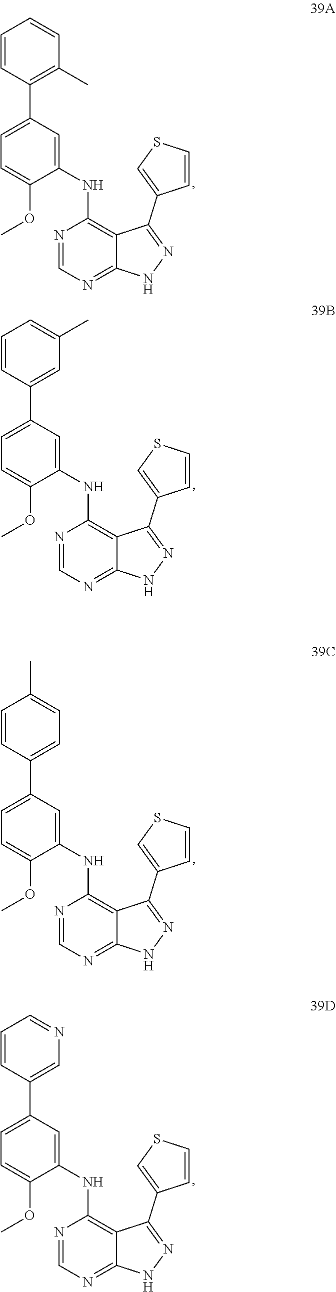

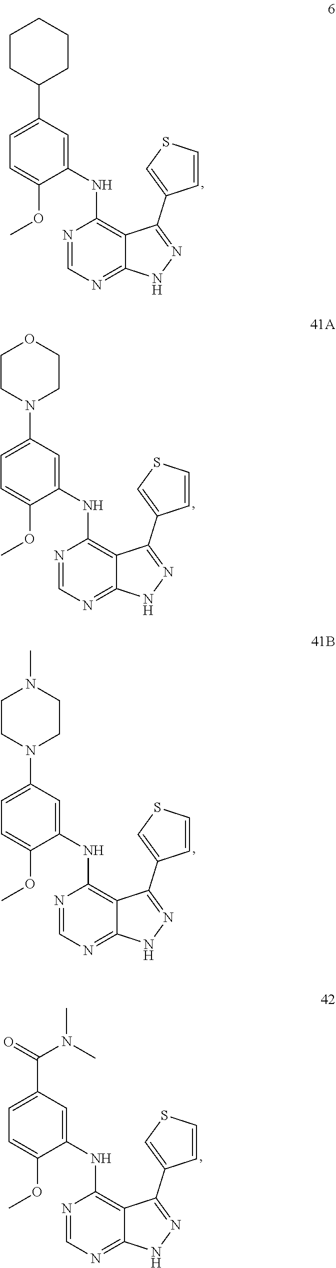 [SMILES: COc1ccc(C2CCCCC2)cc1Nc1ncnc2[nH]nc(-c3ccsc3)c12 ; COc1ccc(N2CCOCC2)cc1Nc1ncnc2[nH]nc(-c3ccsc3)c12 ; COc1ccc(N2CCN(C)CC2)cc1Nc1ncnc2[nH]nc(-c3ccsc3)c12 ; COc1ccc(C(=O)N(C)C)cc1Nc1ncnc2[nH]nc(-c3ccsc3)c12]

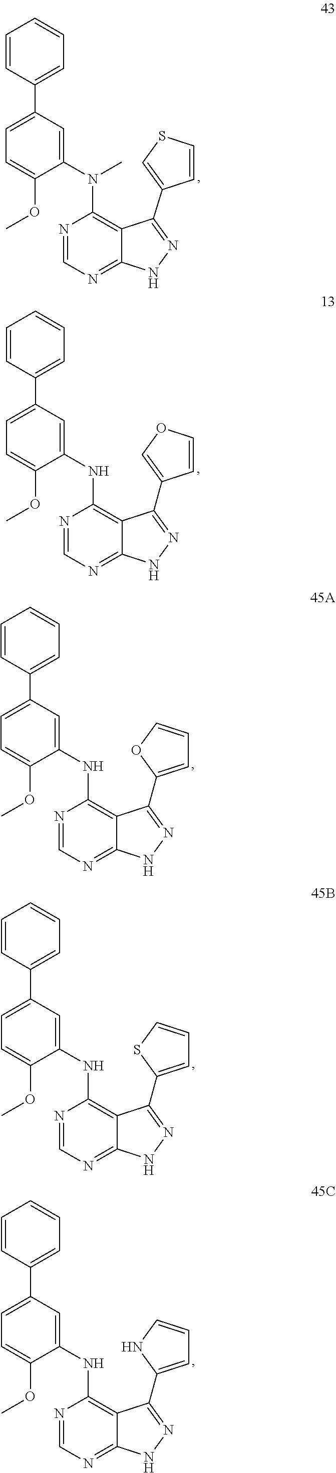 [SMILES: COc1ccc(-c2ccccc2)cc1N(C)c1ncnc2[nH]nc(-c3ccsc3)c12 ; COc1ccc(-c2ccccc2)cc1Nc1ncnc2[nH]nc(-c3ccoc3)c12 ; COc1ccc(-c2ccccc2)cc1Nc1ncnc2[nH]nc(-c3ccco3)c12 ; COc1ccc(-c2ccccc2)cc1Nc1ncnc2[nH]nc(-c3cccs3)c12 ; COc1ccc(-c2ccccc2)cc1Nc1ncnc2[nH]nc(-c3ccc[nH]3)c12]

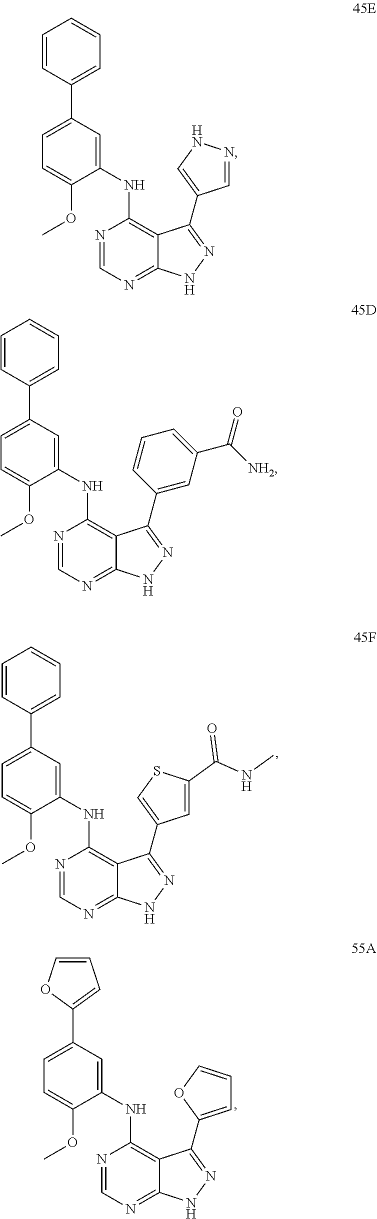

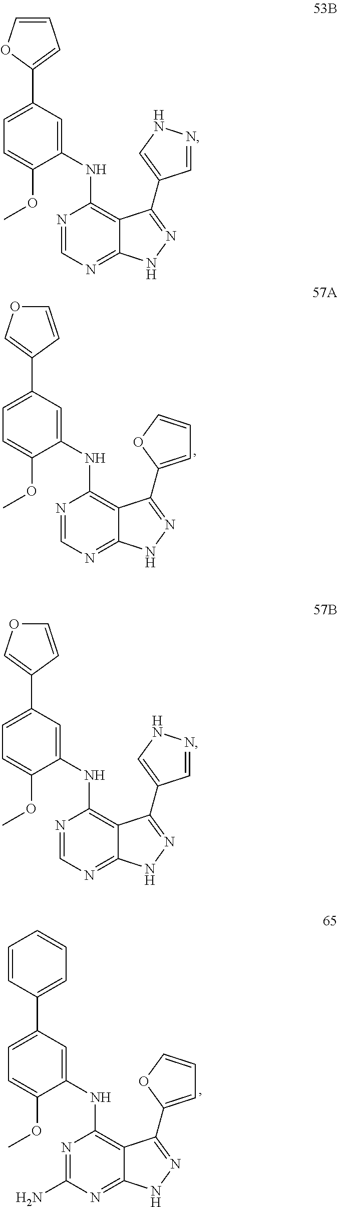 [SMILES: COc1ccc(-c2ccco2)cc1Nc1ncnc2[nH]nc(-c3cn[nH]c3)c12 ; COc1ccc(-c2ccoc2)cc1Nc1ncnc2[nH]nc(-c3ccco3)c12 ; COc1ccc(-c2ccoc2)cc1Nc1ncnc2[nH]nc(-c3cn[nH]c3)c12 ; COc1ccc(-c2ccccc2)cc1Nc1nc(N)nc2[nH]nc(-c3ccco3)c12]

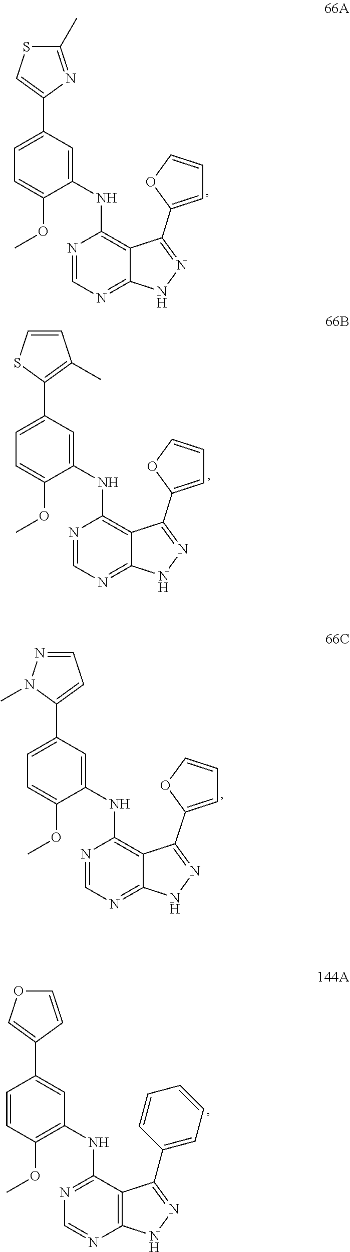 [SMILES: COc1ccc(-c2csc(C)n2)cc1Nc1ncnc2[nH]nc(-c3ccco3)c12 ; COc1ccc(-c2sccc2C)cc1Nc1ncnc2[nH]nc(-c3ccco3)c12 ; COc1ccc(-c2ccnn2C)cc1Nc1ncnc2[nH]nc(-c3ccco3)c12 ; COc1ccc(-c2ccoc2)cc1Nc1ncnc2[nH]nc(-c3ccccc3)c12]

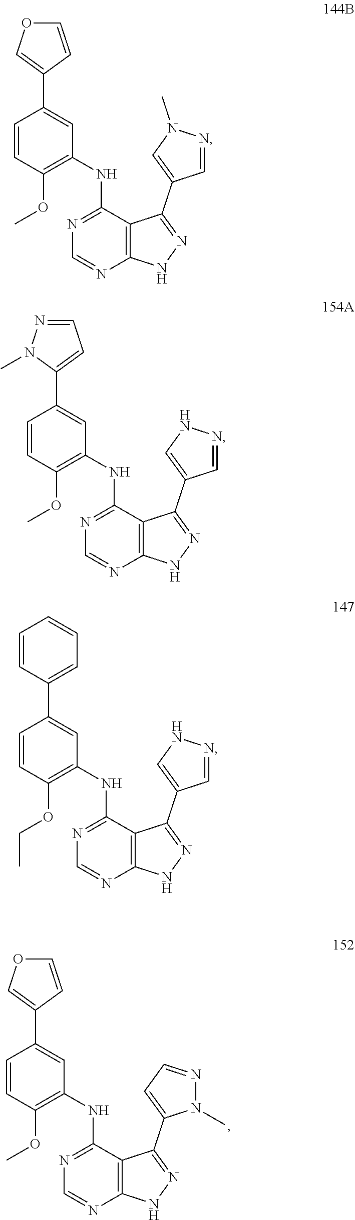 [SMILES: COc1ccc(-c2ccoc2)cc1Nc1ncnc2[nH]nc(-c3cnn(C)c3)c12 ; COc1ccc(-c2ccnn2C)cc1Nc1ncnc2[nH]nc(-c3cn[nH]c3)c12 ; CCOc1ccc(-c2ccccc2)cc1Nc1ncnc2[nH]nc(-c3cn[nH]c3)c12 ; COc1ccc(-c2ccoc2)cc1Nc1ncnc2[nH]nc(-c3ccnn3C)c12]

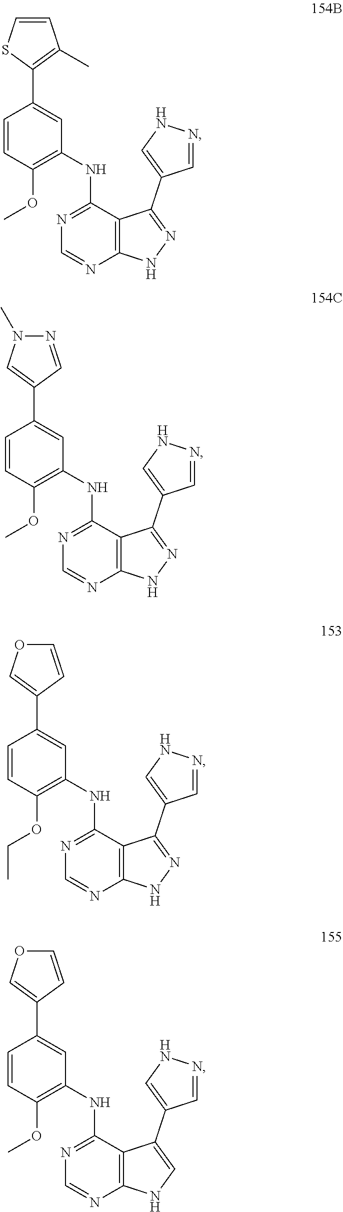 [SMILES: COc1ccc(-c2sccc2C)cc1Nc1ncnc2[nH]nc(-c3cn[nH]c3)c12 ; COc1ccc(-c2cnn(C)c2)cc1Nc1ncnc2[nH]nc(-c3cn[nH]c3)c12 ; CCOc1ccc(-c2ccoc2)cc1Nc1ncnc2[nH]nc(-c3cn[nH]c3)c12 ; COc1ccc(-c2ccoc2)cc1Nc1ncnc2[nH]cc(-c3cn[nH]c3)c12]

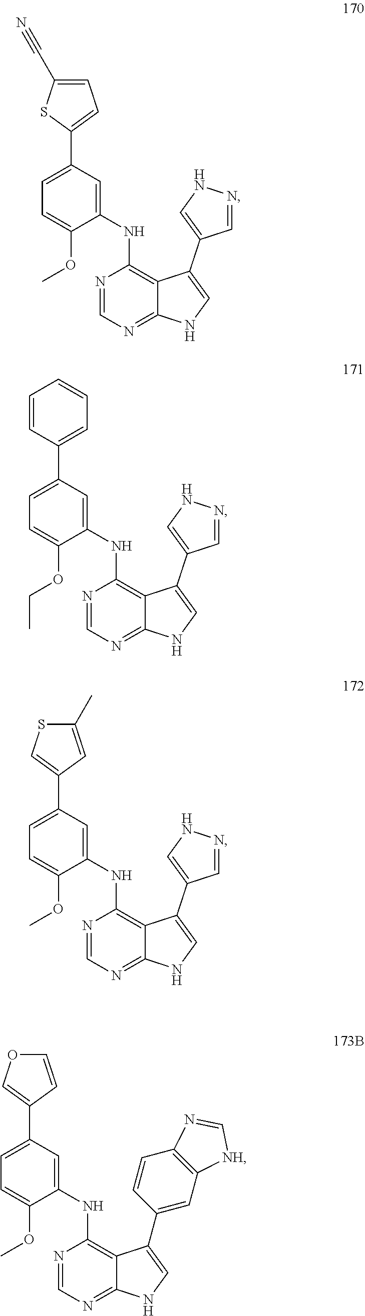 [SMILES: COc1ccc(-c2ccc(C#N)s2)cc1Nc1ncnc2[nH]cc(-c3cn[nH]c3)c12 ; CCOc1ccc(-c2ccccc2)cc1Nc1ncnc2[nH]cc(-c3cn[nH]c3)c12 ; COc1ccc(-c2csc(C)c2)cc1Nc1ncnc2[nH]cc(-c3cn[nH]c3)c12 ; COc1ccc(-c2ccoc2)cc1Nc1ncnc2[nH]cc(-c3ccc4nc[nH]c4c3)c12]

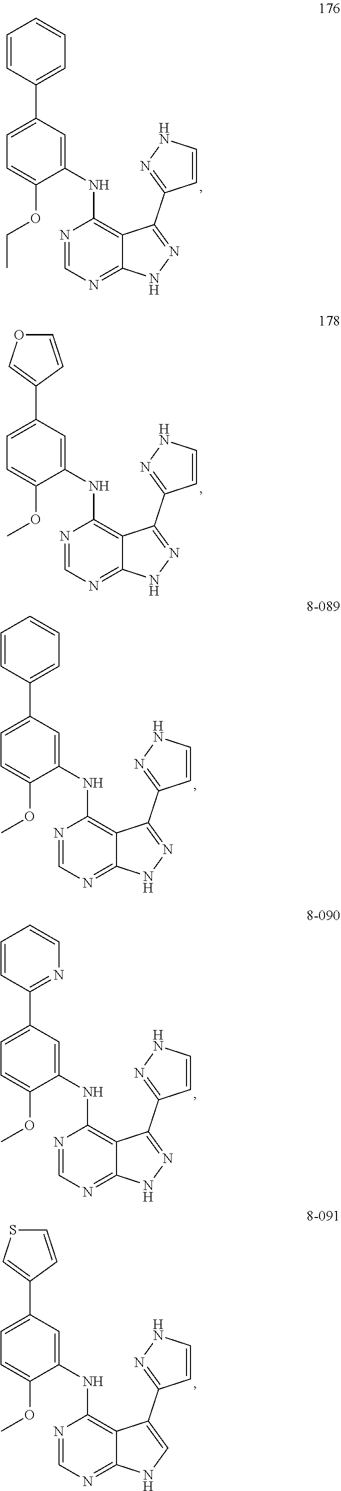 [SMILES: CCOc1ccc(-c2ccccc2)cc1Nc1ncnc2[nH]nc(-c3cc[nH]n3)c12 ; COc1ccc(-c2ccoc2)cc1Nc1ncnc2[nH]nc(-c3cc[nH]n3)c12 ; COc1ccc(-c2ccccc2)cc1Nc1ncnc2[nH]nc(-c3cc[nH]n3)c12 ; COc1ccc(-c2ccccn2)cc1Nc1ncnc2[nH]nc(-c3cc[nH]n3)c12 ; COc1ccc(-c2ccsc2)cc1Nc1ncnc2[nH]cc(-c3cc[nH]n3)c12]

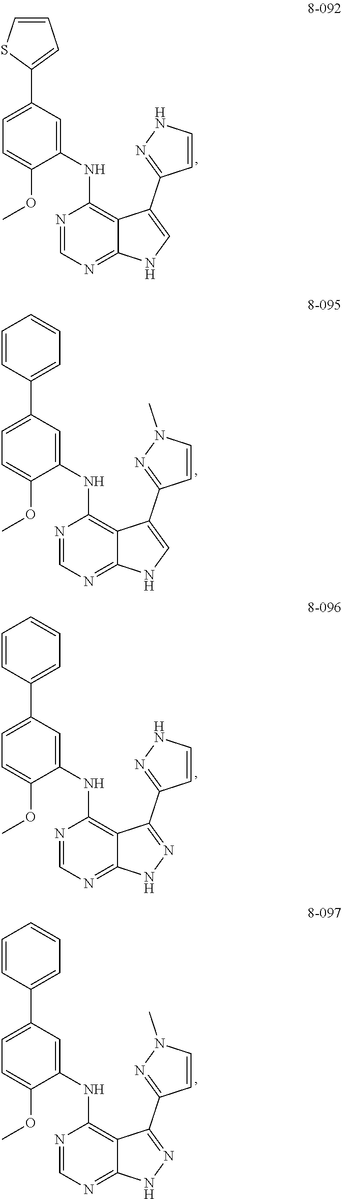 [SMILES: COc1ccc(-c2cccs2)cc1Nc1ncnc2[nH]cc(-c3cc[nH]n3)c12 ; COc1ccc(-c2ccccc2)cc1Nc1ncnc2[nH]cc(-c3ccn(C)n3)c12 ; COc1ccc(-c2ccccc2)cc1Nc1ncnc2[nH]nc(-c3cc[nH]n3)c12 ; COc1ccc(-c2ccccc2)cc1Nc1ncnc2[nH]nc(-c3ccn(C)n3)c12]

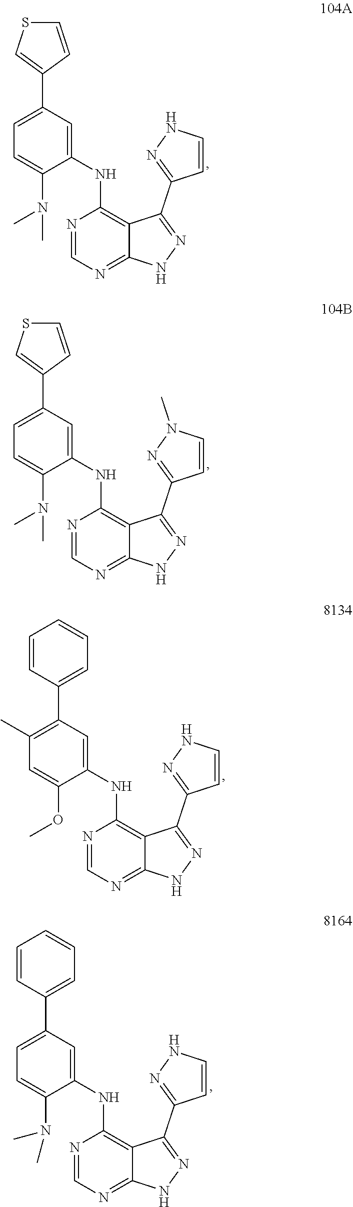 [SMILES: CN(C)c1ccc(-c2ccsc2)cc1Nc1ncnc2[nH]nc(-c3cc[nH]n3)c12 ; CN(C)c1ccc(-c2ccsc2)cc1Nc1ncnc2[nH]nc(-c3ccn(C)n3)c12 ; COc1cc(C)c(-c2ccccc2)cc1Nc1ncnc2[nH]nc(-c3cc[nH]n3)c12 ; CN(C)c1ccc(-c2ccccc2)cc1Nc1ncnc2[nH]nc(-c3cc[nH]n3)c12]

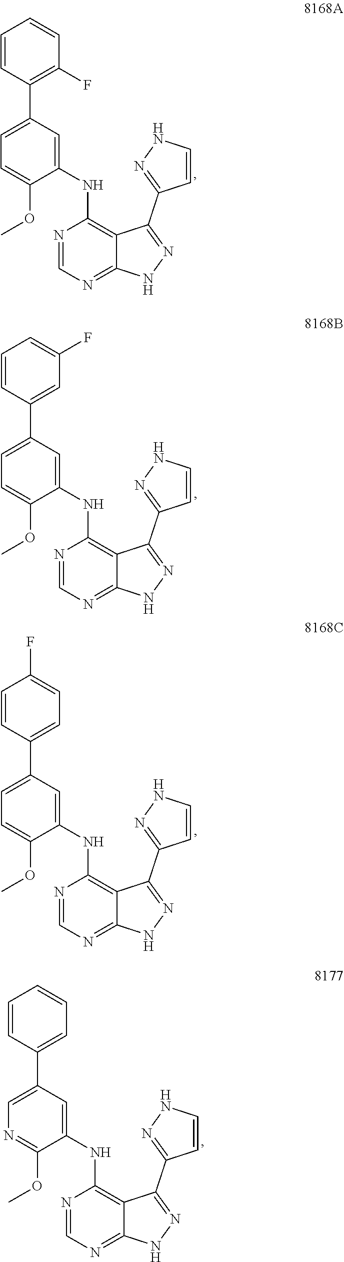 [SMILES: COc1ccc(-c2ccccc2F)cc1Nc1ncnc2[nH]nc(-c3cc[nH]n3)c12 ; COc1ccc(-c2cccc(F)c2)cc1Nc1ncnc2[nH]nc(-c3cc[nH]n3)c12 ; COc1ccc(-c2ccc(F)cc2)cc1Nc1ncnc2[nH]nc(-c3cc[nH]n3)c12 ; COc1ncc(-c2ccccc2)cc1Nc1ncnc2[nH]nc(-c3cc[nH]n3)c12]

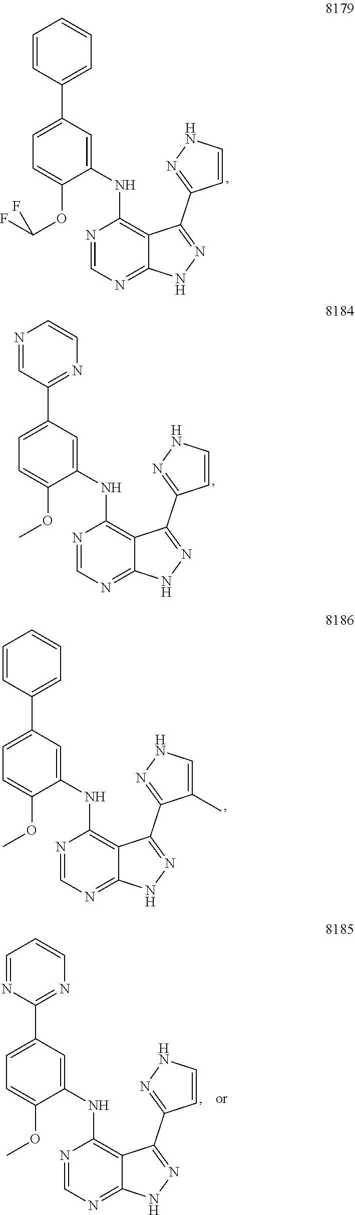 [SMILES: FC(F)Oc1ccc(-c2ccccc2)cc1Nc1ncnc2[nH]nc(-c3cc[nH]n3)c12 ; COc1ccc(-c2cnccn2)cc1Nc1ncnc2[nH]nc(-c3cc[nH]n3)c12 ; COc1ccc(-c2ccccc2)cc1Nc1ncnc2[nH]nc(-c3n[nH]cc3C)c12 ; COc1ccc(-c2ncccn2)cc1Nc1ncnc2[nH]nc(-c3cc[nH]n3)c12]

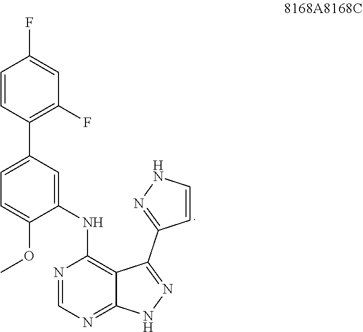 [SMILES: COc1ccc(-c2ccc(F)cc2F)cc1Nc1ncnc2[nH]nc(-c3cc[nH]n3)c12]

45. A pharmaceutical composition comprising a compound of one of claims 1 to 44 or a pharmaceutically acceptable salt thereof, and a pharmaceutically acceptable excipient.

46. The pharmaceutical composition of claim 45, further comprising an anti-cancer agent.

47. A method of treating a disease associated with HER2 activity in a patient in need of such treatment, said method comprising administering a therapeutically effective amount of a compound, or a pharmaceutically acceptable salt thereof, having the formula: 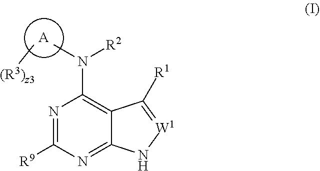
wherein
Ring A is aryl or heteroaryl;
W¹ is N or C(H);
R¹ is hydrogen, -L¹-L²-E, substituted or unsubstituted aryl, or substituted or unsubstituted heteroaryl;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2.

48. A method of treating a disease associated with EGFR activity in a patient in need of such treatment, said method comprising administering a therapeutically effective amount of a compound, or a pharmaceutically acceptable salt thereof, having the formula: 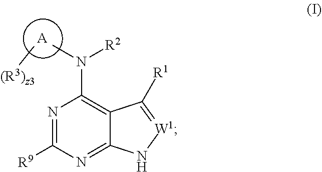
wherein;
Ring A is aryl or heteroaryl;
W¹ is N or C(H);
R¹ is hydrogen, -L¹-L²-E, substituted or unsubstituted aryl, or substituted or unsubstituted heteroaryl;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2.

49. A method of treating cancer in a patient in need of such treatment, said method comprising administering a therapeutically effective amount of a compound, or a pharmaceutically acceptable salt thereof, having the formula: 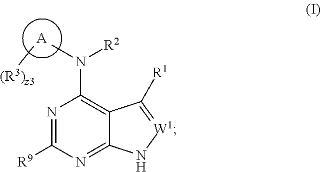
wherein;
Ring A is aryl or heteroaryl;
W¹ is N or C(H);
R¹ is hydrogen, -L¹-L²-E, substituted or unsubstituted aryl, or substituted or unsubstituted heteroaryl;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2.

50. The method of claim 49, wherein the cancer is resistant to a HER2 inhibitor.

51. The method of claim 49, wherein the cancer is resistant to an EGFR inhibitor.

52. A method of inhibiting HER2 activity, said method comprising contacting HER2 with an effective amount of a compound, or a pharmaceutically acceptable salt thereof, having the formula: 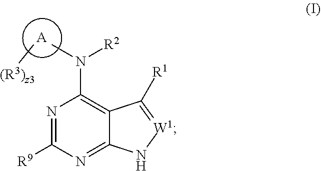
wherein;
Ring A is aryl or heteroaryl;
W¹ is N or C(H);
R¹ is hydrogen, -L¹-L²-E, substituted or unsubstituted aryl, or substituted or unsubstituted heteroaryl;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2.

53. The method of claim 52, wherein HER2 is in an active conformation.

54. The method of claim 53, wherein HER2 is in a HER2-HER3 heterodimer.

55. A method of inhibiting EGFR activity, said method comprising contacting EGFR with an effective amount of a compound, or a pharmaceutically acceptable salt thereof, having the formula: 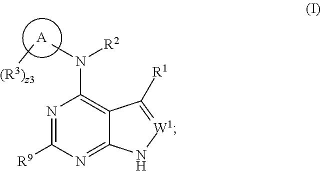
wherein;
Ring A is aryl or heteroaryl;
W¹ is N or C(H);
R¹ is hydrogen, -L¹-L²-E, substituted or unsubstituted aryl, or substituted or unsubstituted heteroaryl;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L²is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2.

56. The method of claim 55, wherein EGFR is in an active conformation.

57. A method of claim 47, wherein the compound has the formula: 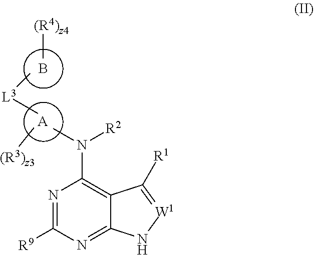
wherein
Ring B is cycloalkyl, heterocycloalkyl, aryl, or heteroaryl;
L³is a bond, —S(O)₂—, —NR⁸—, —O—, —S—, —C(O)—, —C(O)NR⁸—, —NR⁸C(O)—, —NR⁸C(O)NH—, —NHC(O)NR⁸—, —C(O)O—, —OC(O)—, substituted or unsubstituted alkylene, or substituted or unsubstituted heteroalkylene;
R⁴ is independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z4 is an integer from 0 to 5;
Each R^{4A}, R^{4B}, R^{4C}, R^{4D}, R^{8A}, R^{8B}, R^{8C}, and R^{8D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl
each X⁴ and X⁸ are independently —F, —Cl, —Br, or —I;
n4 and n8 are independently an integer from 0 to 4; and
m4, m8, v4, and v8, are independently an integer from 1 to 2.

58. A method of claim 47, wherein the compound has the formula:

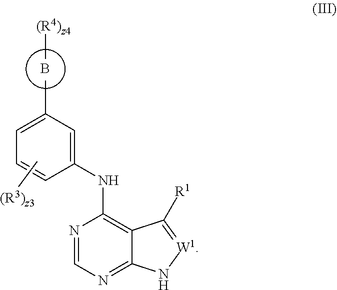

59. A method of claim 47, wherein the compound has the formula:

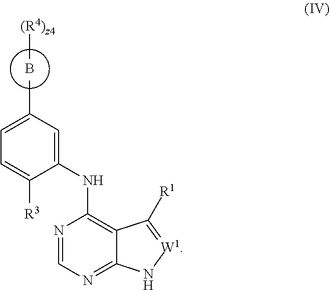

60. The method of claim 57, wherein R⁴ is independently halogen, —CX⁴₃, —CHX⁴₂, —CH₂X⁴, —OCX⁴₃, —OCH₂X⁴, —OCHX⁴₂, or —CN.

61. The method of claim 57, wherein R⁴ is independently halogen, —CX⁴₃, —CHX⁴₂, or —CH₂X⁴.

62. The method of claim 57, wherein R⁴ is independently halogen.

63. The method of claim 47, wherein the compound has the formula:

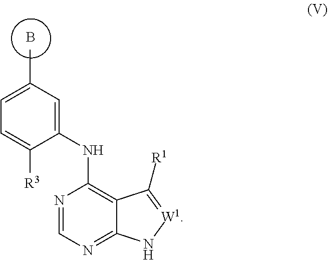

64. The method of claim 47, wherein W¹ is C(H).

65. The method of claim 47, wherein W¹ is N.

66. The method of claim 47, wherein R³ is an unsubstituted heteroalkyl.

67. The method of claim 47, wherein R³ is unsubstituted 2 to 5 membered heteroalkyl.

68. The method of claim 47, wherein R³ is —OCH₃, —OCH₂CH₃, —N(CH₃)₂, —NH₂, —NH(CH₃), —N(CH₂CH₃)₂, —NH(CH₂CH₃), —OCX³₃, —OCH₂X³, —OCHX³₂, or —SH.

69. The method of claim 57, wherein Ring B is substituted or unsubstituted aryl or heteroaryl.

70. The method of claim 57, wherein Ring B is substituted or unsubstituted phenyl or substituted or unsubstituted 5 to 6 membered heteroaryl.

71. The method of claim 57, wherein Ring B is substituted or unsubstituted phenyl.

72. The method of claim 57, wherein Ring B is substituted or unsubstituted 5 to 6 membered heteroaryl.

73. The method of claim 57, wherein Ring B is substituted or unsubstituted pyrazinyl, pyrimidinyl, furanyl, thienyl, pyrrolyl, imidazolyl, pyrazolyl, oxazolyl, isoxazolyl, or thiazolyl.

74. The method of claim 47, wherein R¹ is substituted or unsubstituted aryl or substituted or unsubstituted heteroaryl.

75. The method of claim 47, wherein R¹ is substituted or unsubstituted phenyl or substituted or unsubstituted 5 to 6 membered heteroaryl.

76. The method of claim 47, wherein R¹ is substituted or unsubstituted phenyl.

77. The method of one of claims 47 to 73, wherein R¹ is an unsubstituted phenyl.

78. The method of claim 47, wherein R¹ is a substituted or unsubstituted 5 to 6 membered heteroaryl.

79. The method of claim 47, wherein R¹ is an unsubstituted 5 to 6 membered heteroaryl.

80. The method of claim 47, wherein R¹ is substituted or unsubstituted pyridinyl, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted oxazolyl, substituted or unsubstituted isoxazolyl, or substituted or unsubstituted thiazolyl.

81. The method of claim 47, wherein R¹ is an unsubstituted furanyl, unsubstituted thienyl, unsubstituted pyrrolyl, unsubstituted imidazolyl, unsubstituted pyrazolyl, unsubstituted oxazolyl, unsubstituted isoxazolyl, or unsubstituted thiazolyl.

82. The method of claim 47, wherein R¹ is -L¹-L²-E.

83. The method of claim 47, wherein L¹ is a bond, —C(O)—, substituted or unsubstituted alkylene, or substituted or unsubstituted heteroalkylene.

84. The method of claim 47, wherein L¹ is a substituted or unsubstituted C₁-C₄ alkylene.

85. The method of claim 47, wherein L¹ is —C(O)CH₂CH₂CH₂—, —C(O)CH₂CH₂—, or —C(O)CH₂—.

86. The method of claim 47, wherein L² is —NR⁷—, substituted or unsubstituted heteroalkylene, or substituted or unsubstituted heterocycloalkylene.

87. The method of claim 47, wherein L² is —NH—.

88. The method of claim 47, wherein E is a covalent cysteine modifier moiety.

89. The method of claim 47, wherein E is: 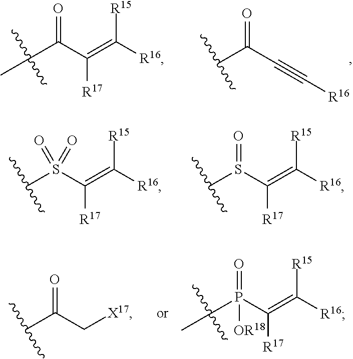
R¹⁵ is independently hydrogen, halogen, CX¹⁵₃, —CHX¹⁵₂, —CH₂X¹⁵, —CN, —SOₙ₁₅R^{15D, —SO}ᵥ₁₅R^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁶ is independently hydrogen, halogen, CX¹⁶₃, —CHX¹⁶₂, —CH₂X¹⁶, —CN, —SOₙ₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;
each X, X¹⁵, X¹⁶, X¹⁷ and X¹⁸ is independently —F, —Cl, —Br, or —I;
n15, n16, n17, v15, v16, and v17, are independently an integer from 0 to 4; and
m15, m16, and m17 are independently and integer from 1 to 2.

90. The method of claim 89, wherein R¹⁵, R¹⁶, R¹⁷, and R¹⁸ are hydrogen.

91. The method of claim 89, wherein E is:

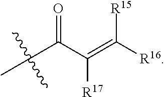

92. The method of claim 91, wherein
R¹⁵ is hydrogen;
R¹⁶ is hydrogen, —CH₃, or —CH₂NR^{16A}R^{16B};
R¹⁷ is hydrogen; and
R^{16A} and R^{16B} are independently hydrogen or unsubstituted alkyl.

93. The method of claim 92, wherein R^{16A} and R^{16B} are independently unsubstituted methyl.

94. The method of claim 91, wherein
R¹⁵ is hydrogen;
R¹⁶ is hydrogen;
R¹⁷ is hydrogen, —CH₃, or —CH₂NR^{17A}R^{17B}; and
R^{17A} and R^{17B} are independently hydrogen or unsubstituted alkyl.

95. The method of claim 94, wherein R^{17A} and R^{17B} are independently unsubstituted methyl.

96. The compound of claim 91, wherein
R¹⁵ is hydrogen, —CH₃, or —CH₂NR^{15A}R^{15B};
R¹⁶ is hydrogen;
R¹⁷ is hydrogen; and
R^{15A} and R^{15B} are independently hydrogen or unsubstituted alkyl.

97. The method of claim 96, wherein R^{15A} and R^{15B} are independently unsubstituted methyl.

98. An EGFR protein covalently bonded to a compound having the formula: 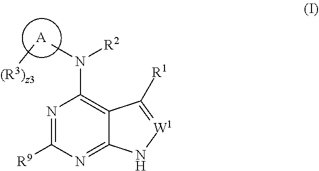
wherein
Ring A is aryl or heteroaryl;
W¹ is N or C(H)
R¹ is -L¹-L²-E;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2.

99. A HER2 protein covalently bonded to a compound having the formula: 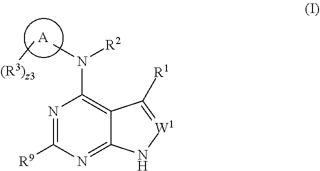
wherein
Ring A is aryl or heteroaryl;
W¹ is N or C(H)
R¹ is -L¹-L²-E;
R² is hydrogen, —CX²₃, —CHX²₂, —CH₂X², substituted or unsubstituted alkyl, or substituted or unsubstituted heteroalkyl;
R³ is independently halogen, —CX³₃, —CHX³₂, —CH₂X³, —OCX⁴₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 4;
L¹ is a bond, —S(O)₂—, —S(O)₂-Ph-, —NR⁶—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —S(O)₂-Ph-, —NR⁷—, —O—, —S—, —C(O)—, —C(O)NR⁷—, —NR⁷C(O)—, —NR⁷C(O)NH—, —NHC(O)NR⁷—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
each R^{3A}, R^{3B}, R^{3C}, R^{3D}, R^{6A}, R^{6B}, R^{6C}, R^{6D}, R^{7A}, R^{7B}, R^{7C}, R^{7D}, R^{9A}, R^{9B}, R^{9C}, and R^{9D} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
each X, X³, X⁶, X⁷, and X⁹ is independently —F, —Cl, —Br, or —I;
n3, n6, n7, and n9 are independently an integer from 0 to 4; and
m3, m6, m7, m9, v3, v6, v7, and v9, are independently an integer from 1 to 2.
